(19) United States
(12) Patent Application Publication
(10) Pub. No.: US 2026/0234257 A1
(43) Pub. Date: Aug. 13, 2026

(54) T CELL ENGAGERS AND METHODS OF USE THEREOF

(71) Applicant: Janux Therapeutics, Inc., San Diego, CA (US)

(72) Inventors: David CAMPBELL, San Diego, CA (US); Thomas R. DIRAIMONDO, San Diego, CA (US); John LAUDENSLAGER, La Mesa, CA (US)

(21) Appl. No.: 19/652,551

(22) Filed: Apr. 20, 2026

Related U.S. Application Data
(63) Continuation of application No. PCT/US2026/014068, filed on Feb. 5, 2026.
(60) Provisional application No. 63/754,282, filed on Feb. 5, 2025.

(51) Int. Cl. C07K 16/28; A61K 38/00; A61P 37/04; C12N 5/00; C12N 5/0783

(52) U.S. Cl. CPC C07K 16/2809; A61P 37/04; C12N 5/0018; C12N 5/0636; A61K 38/00; C07K 2317/55; C07K 2317/565; C07K 2317/622; C12N 2501/515

(57) ABSTRACT

The present disclosure relates to polypeptide molecules that specifically bind T cell receptor beta and compositions and methods comprising and employing the same.

### CROSS REFERENCE TO RELATED APPLICATIONS

This application is a continuation of PCT Application No. PCT/US2026/014068, filed Feb. 5, 2026, which claims the benefit of and the priority from U.S. Provisional Application No. 63/754,282, filed Feb. 5, 2025, the disclosure of each of which is incorporated by reference in its entirety.

### FIELD OF THE DISCLOSURE

The present disclosure relates to polypeptide molecules that specifically bind T cell receptor beta (TCRβ), as well as compositions and methods of using said polypeptide molecules.

### REFERENCE TO AN ELECTRONIC SEQUENCE LISTING

The contents of the electronic sequence listing (JANX_005_02US_SeqList_ST26.xml; Size: 2,561,106 bytes; and Date of Creation: Apr. 20, 2026) are herein incorporated by reference in their entirety.

### BACKGROUND

T cell engagers are a class of immunotherapeutic molecules that redirect the patient's immune response to recognize and kill target cells (e.g., B cells expressing CD19). While T cell engagers have shown to elicit potent immune activity, these therapeutics have faced challenges due to overactivation of the immune system leading to cytokine release syndrome (CRS), on-target, healthy tissue toxicities, T cell exhaustion, as well as depletion of effector T cell populations with minimal expansion of memory T cell populations that can limit duration of immune activity.

Thus, there is a need in the art to improve the design of T cell engagers to improve the duration of immune activity as well as to reduce the toxicity and side effects associated with T cell engagers.

### SUMMARY OF THE INVENTION

The present disclosure provides T cell engagers, and compositions and methods comprising or employing the same. In some embodiments, the T cell engager is a polypeptide molecule comprising an anti-TCRβ-binding domain. In some embodiments, the T cell engager is a polypeptide molecule comprising an anti-TCRβC-binding domain. In some embodiments, the T cell engager is a polypeptide molecule comprising an anti-TCRβC1-binding domain. In some embodiments, the T cell engager is a polypeptide molecule comprising an anti-TCRβC2-binding domain. In some embodiments, the T cell engager is a polypeptide molecule comprising an anti-TCRβV-binding domain. In some embodiments, the T cell engager is a polypeptide molecule comprising an anti-TCRβV6-5-binding domain. In some embodiments, the T cell engager is a polypeptide molecule comprising an anti-TCRβV12-3-binding domain.

In some embodiments, the T cell engager is a polypeptide molecule comprising a first binding domain and a second binding domain. In some embodiments, the first binding domain comprises an anti-TCRβ-binding domain (e.g., an anti-TCRβC-binding domain or an anti-TCRβV-binding domain) and the second binding domain comprises an anti-tumor-associated antigen (TAA)-binding domain (e.g., an anti-CD19 binding domain). In some embodiments, a linker connects the first binding domain and the second binding domain. In some embodiments, the first binding domain comprises an scFv and the second binding domain comprises a Fab.

In some embodiments, the T cell engager is a polypeptide molecule comprising an anti-TCRβ-binding domain, an anti-TAA-binding domain, and an Fc region. In some embodiments, the polypeptide molecule comprises one or more linkers connecting the anti-TCRβ-binding domain, the anti-TAA-binding domain, and the Fc region.

In some embodiments, the present disclosure comprises treating a disease, disorder, or condition in a subject in need thereof by administering the polypeptide molecules disclosed herein to the subject. In some embodiments, the disease, disorder, or condition is cancer. In some embodiments, the disease, disorder, or condition is an autoimmune disease.

In some embodiments, the present disclosure provides methods for expanding T cells (e.g., effector T cells or memory T cells) in a subject by administering the polypeptide molecules disclosed herein.

In some embodiments, the present disclosure provides methods for depleting target cells (e.g., tumor cells or B cells) in a subject by administering the polypeptide molecules disclosed herein.

### BRIEF DESCRIPTION OF THE DRAWINGS

FIGS. 1A-1B provide schematics of exemplary polypeptide molecules with the C-terminus of the anti-TCRβ scFv fused to the N-terminal heavy chain (HC) of the anti-TAA Fab-binding domain (FIG. 1A) and the C-terminus of the anti-TCRβ scFv fused to the N-terminal light chain (LC) of the anti-TAA Fab-binding domain (FIG. 1B). TAA, tumor-associated antigen.

FIGS. 2A-2C show tumor cell viability (left y-axis) and IFNγ (FIG. 2A), TNF (FIG. 2B), or IL-2 (FIG. 2C) concentration (right y-axis) as a function of PSMA-CD3 polypeptide molecule (PC43) concentration in a co-culture of CD8+ T cells and LNCaP tumor cells.

FIGS. 3A-3C show tumor cell viability (left y-axis) and IFNγ (FIG. 3A), TNF (FIG. 3B), or IL-2 (FIG. 3C) concentration (right y-axis) as a function of PSMA-CD3 polypeptide molecule (PC44) concentration in a co-culture of CD8+ T cells and LNCaP tumor cells.

FIGS. 4A-4C show tumor cell viability (left y-axis) and IFNγ (FIG. 4A), TNF (FIG. 4B), or IL-2 (FIG. 4C) concentration (right y-axis) as a function of PSMA-TCRβC1 polypeptide molecule (PC01) concentration in a co-culture of CD8+ T cells and LNCaP tumor cells.

FIGS. 5A-5C show tumor cell viability (left y-axis) and IFNγ (FIG. 5A), TNF (FIG. 5B), or IL-2 (FIG. 5C) concentration (right y-axis) as a function of PSMA-TCRβC1 polypeptide molecule (PC02) concentration in a co-culture of CD8+ T cells and LNCaP tumor cells.

FIGS. 6A-6C show tumor cell viability (left y-axis) and IFNγ (FIG. 6A), TNF (FIG. 6B), or IL-2 (FIG. 6C) concentration (right y-axis) as a function of PSMA-TCRβC2 polypeptide molecule (PC03) concentration in a co-culture of CD8+ T cells and LNCaP tumor cells.

FIGS. 7A-7C show tumor cell viability (left y-axis) and IFNγ (FIG. 7A), TNF (FIG. 7B), or IL-2 (FIG. 7C) concentration (right y-axis) as a function of PSMA-TCRβC2 polypeptide molecule (PC04) concentration in a co-culture of CD8+ T cells and LNCaP tumor cells.

FIGS. 8A-8C show tumor cell viability (left y-axis) and IFNγ (FIG. 8A), TNF (FIG. 8B), or IL-2 (FIG. 8C) concentration (right y-axis) as a function of PSMA-TCRβV6-5 polypeptide molecule (PC05) concentration in a co-culture of CD8+ T cells and LNCaP tumor cells.

FIGS. 9A-9C show tumor cell viability (left y-axis) and IFNγ (FIG. 9A), TNF (FIG. 9B), or IL-2 (FIG. 9C) concentration (right y-axis) as a function of PSMA-TCRβV6-5 polypeptide molecule (PC06) concentration in a co-culture of CD8+ T cells and LNCaP tumor cells.

FIGS. 10A-10C show tumor cell viability (left y-axis) and IFNγ (FIG. 10A), TNF (FIG. 10B), or IL-2 (FIG. 10C) concentration (right y-axis) as a function of PSMA-TCRβV12-3/4 polypeptide molecule (PC07) concentration in a co-culture of CD8+ T cells and LNCaP tumor cells.

FIGS. 11A-11C show tumor cell viability (left y-axis) and IFNγ (FIG. 11A), TNF (FIG. 11B), or IL-2 (FIG. 11C) concentration (right y-axis) as a function of PSMA-TCRβV12-3/4 polypeptide molecule (PC08) concentration in a co-culture of CD8+ T cells and LNCaP tumor cells.

FIGS. 12A-12B show tumor cell viability (left y-axis) and IFNγ (FIG. 12A) or IL-2 (FIG. 12B) concentration (right y-axis) as a function of PSMA-TCRβV5-5/6 polypeptide molecule (PC09) concentration in a co-culture of CD8+ T cells and LNCaP tumor cells.

FIGS. 13A-13B show tumor cell viability (left y-axis) and IFNγ (FIG. 13A) or IL-2 (FIG. 13B) concentration (right y-axis) as a function of PSMA-TCRβV5-5/6 polypeptide molecule (PC10) concentration in a co-culture of CD8+ T cells and LNCaP tumor cells.

FIGS. 14A-14C show tumor cell viability (left y-axis) and IFNγ (FIG. 14A), TNF (FIG. 14B), or IL-2 (FIG. 14C) concentration (right y-axis) as a function of PSMA-TCRβV20-1 polypeptide molecule (PC12) concentration in a co-culture of CD8+ T cells and LNCaP tumor cells.

FIGS. 15A-15B show tumor cell viability (FIG. 15A) and IFNγ concentration (FIG. 15B) as a function of polypeptide molecule PC11 (PSMA-TCRβV20-1), PC12 (PSMA-TCRβV20-1), PC06 (PSMA-TCRβV6-5), or PC08 (PSMA-TCRβV12-3/4) concentration in a co-culture of CD8+ T cells and LNCaP tumor cells.

FIGS. 15C-15D show IL-2 (FIG. 15C) and TNF (FIG. 15D) concentration as a function of polypeptide molecule PC11 (PSMA-TCRβV20-1), PC12 (PSMA-TCRβV20-1), PC06 (PSMA-TCRβV6-5), or PC08 (PSMA-TCRβV12-3/4) concentration in a co-culture of CD8+ T cells and LNCaP tumor cells.

FIG. 16 shows an exemplary flow cytometry plot of the gating strategy used to determine human T cell memory populations. Tn=naïve T cells; Tscm=stem-cell memory; Ttm=transition cell memory; Tcm=central memory; Tem=effector memory; L-Tem=late effector memory; and Temra=effector memory re-expressing CD45RA.

FIGS. 17A-17B show results of the saturating mutagenesis selection for single pointed mutations on 3LCDRs (FIG. 17A) and 3HCDRs (FIG. 17B) of a wildtype TRBV6-5 binder. The tolerance of a single pointed mutation is determined by average OD of substitution AA/WT AA with a cutoff value of 0.34. A single pointed mutation with a value higher than 0.34 is determined to be tolerated.

FIGS. 18A-18B show results of the saturating mutagenesis selection for single pointed mutations on 3LCDRs (FIG. 18A) and 3HCDRs (FIG. 18B) of a wildtype TRBV12-3 binder. The tolerance of a single pointed mutation is determined by average OD of substitution AA/WT AA with a cutoff value of 0.5. A single pointed mutation with a value higher than 0.5 is determined to be tolerated.

FIGS. 19A-19B display exemplary molecule formats for TRBV binding domains. FIG. 19A shows exemplary formats for scFv and Fab binding domains. FIG. 19B shows exemplary formats for bispecific molecules.

FIGS. 20A-20C shows ELISA binding to human (FIG. 20A) and cyno (FIG. 20B) antigens for exemplary mutated Fab constructs against TRBV6-5. Summarized results are displayed in FIG. 20C. Results show that affinity maturation of the original T cell binder strengthened human TRBV6-5 binding affinity by 2-fold but drastically strengthened cyno TRBV6-2 binding by more than 800-fold.

FIGS. 21A-21F show ELISA binding results for exemplary anti-TRBV6-5 binders: CLY-008, CLY-010, and CLY-013. FIG. 21A shows results for TRBV-Fc proteins, CLY-001 to CLY-005, at 5 μg/mL binding CLY-008 at concentrations from 0-5 μM. FIG. 21B summarizes the results of FIG. 21A. FIG. 21C shows results for TRBV-Fc proteins, CLY-001 to CLY-005, at 5 μg/mL binding CLY-010 at concentration from 0-5 μM. FIG. 21D summarizes the results of FIG. 21C. FIG. 21E shows results for TRBV-Fc proteins, CLY-001 to CLY-005, at 5 μg/mL binding CLY-013 at concentrations from 0-5 μM. FIG. 21F summarizes the results of FIG. 21E.

FIGS. 22A-22H show ELISA binding results for exemplary anti-CD19 and anti-TRBV bi-specific binders, CLY-018 to CLY-024. FIG. 22A shows a summary of binding results for binders CLY-018 to CLY-024 against human CD19 (5 ug/mL) at concentrations from 0-5 μM. FIG. 22B summarizes the results of FIG. 22A. FIG. 22C shows a summary of binding results for binders CLY-018 to CLY-024 against cyno CD19 (5 ug/mL) at concentrations from 0-5 μM. FIG. 22D summarizes the results of FIG. 22C. FIG. 22E shows a summary of binding results for binders CLY-018 to CLY-024, against human TRBV6-5 at 5 μg/mL. FIG. 22F summarizes the results of FIG. 22E. FIG. 22G shows a summary of binding results for binders CLY-018 to CLY-024 against cyno TRBV6-2 at 5 μg/mL. FIG. 22H summarizes the results of FIG. 22G.

FIG. 22I shows a summary of binding results for exemplary TRBV12-3 binders, CLY-034 to CLY-037, against human TRBV12-3. FIG. 22J summarizes the results of FIG. 22I. FIG. 22K shows a summary of binding results for CLY-034 to CLY-037 against cyno TRBV12-3. FIG. 22L summarizes the results of FIG. 22K.

FIGS. 23A-23D show results of human cell binding of exemplary anti-CD19 and anti-TRBV bi-specific binders, CLY-018 to CLY-024, by flow cytometry. FIG. 23A shows the cell binding results by flow cytometry to human B cells. FIG. 23B summarizes the results of FIG. 23A. FIG. 23C shows the flow cytometry cell binding results to human T cells. FIG. 23D summarizes the results of FIG. 23C.

FIGS. 24A-24D show results of cyno cell binding of exemplary anti-CD19 and anti-TRBV bi-specific binders, CLY-018 to CLY-024, by flow cytometry. FIG. 24A shows binding results of the binders to cyno B cells. FIG. 24B summarizes the results of FIG. 24A. FIG. 24C shows binding results of the binders to cyno T cells. FIG. 24D summarizes the results of FIG. 24C.

FIGS. 25A-FIG. 25U show results of functionality assays for exemplary anti-CD19 and anti-TRBV bi-specific binders CLY-018 to CLY-024. FIG. 25A shows normalized B cell count for human PBMC sample and the exemplary binders. FIG. 25B shows results for CD8+TRBV+ for human PBMC sample and the exemplary binders. FIG. 25C shows results for non-Treg CD4+TRBV+ for human PBMC sample and the exemplary binders. FIG. 25D shows results for T regs for human PBMC sample and the exemplary binders. FIG. 25E summarizes the EC50 values for human B cell killing. FIG. 25F shows expansion of CD8+TRBV+ for CLY-023 binders from human PBMC sample. FIG. 25G shows expansion of CD8+TRBV+ for CLY-018 binders from human PBMC sample. FIG. 25H shows expansion of CD8+TRBV+ for CLY-019 binders from human PBMC sample. FIG. 25I shows expansion of CD8+TRBV+ for CLY-020 binders from human PBMC sample. FIG. 25J shows expansion of CD8+TRBV+ for CLY-021 binders from human PBMC sample. FIG. 25K shows expansion of CD8+TRBV+ for CLY-022 binders from human PBMC sample. FIG. 25L shows B cell count after treatment of cyno PBMC (donor “326”) with CLY-020, CLY-021, or CLY-024. FIG. 25M summarizes the results of FIG. 25L. FIG. 25N shows B cell count after treatment of cyno PBMC (donor “1611”) with CLY-020, CLY-021, or CLY-024. FIG. 25O summarizes the results of FIG. 25N. FIG. 25P shows CD8+TRBV+ fold change for cyno PBMC (donor “326”) treated with CLY-020, CLY-021, or CLY-024. FIG. 25Q shows CD8+TRBV+ fold change for cyno PBMC (donor “1611”) treated with CLY-020, CLY-021, or CLY-024. FIG. 25R shows CD4+TRBV+ fold change for cyno PBMC (donor “326”) treated with CLY-020, CLY-021, or CLY-024. FIG. 25S shows CD4+TRBV+ fold change for cyno PBMC (donor “1611”) treated with CLY-020, CLY-021, or CLY-024. FIG. 25T shows CD8+TRBV+ T cell fold change for cyno PBMC donor “326” treated with CLY-020. FIG. 25U shows CD8+TRBV+ T cell fold change for cyno PBMC donor “1611” treated with CLY-020.

FIGS. 26A-26F show functionality results for assays utilizing exemplary anti-CD19 and anti-TRBV bi-specific binders CLY-023, CLY-018, CLY-019, and CLY-022. FIG. 26A shows average TRBV+CD8+ T cell count for 3 donors treated with CLY-023, CLY-018, CLY-019, or CLY-022 over a 7-day culture. FIG. 26B shows average TRBV+CD4+ T cell count for 3 donors treated with CLY-023, CLY-018, CLY-019, or CLY-022 over a 7-day culture. FIG. 26C shows expansion of TRBV+CD8+ T cells after 7 day culture with CLY-022, CLY-019, CLY-018, or CLY-023 enriched for TRBV6-1, 6-2, 6-5, and 6-9 (n=3 donor CD8+ T cells), of note is that CLY-022 also expanded TRBV10-3 T cells. FIG. 26D shows expansion of TRBV+CD4+ T cells after 7 day culture with CLY-022, CLY-019, CLY-018, or CLY-023 enriched for TRBV6-1, 6-2, 6-5, and 6-9 (n=2-3 donor CD4+ T cells), of note is that CLY-022 also expanded TRBV10-3 T cells. FIG. 26E shows expansion of TRAV+CD8+ T cells after 7 day culture with CLY-022, CLY-019, CLY-018, or CLY-023 indicating no change in relative abundance of TRAVs with TRBV stimulation (n=2-3 donor CD4+ T cells). FIG. 26F shows expansion of TRAV+CD4+ T cells after 7 day culture with CLY-022, CLY-019, CLY-018, or CLY-023 indicating no change in relative abundance of TRAVs with TRBV stimulation (n=2-3 donor CD4+ T cells).

FIGS. 27A-27R show results of in vivo activity of exemplary anti-CD19 and anti-TRBV bi-specific binders in a cynomolgus monkey model. FIG. 27A shows the plasma concentration of CLY-023 for monkeys treated with CLY-023. FIG. 27B shows CD20+ B cell count in blood of monkeys treated with CLY-023. Of note is that minimal B cell depletion was observed in monkeys dosed up to 1 mg/kg IV. FIG. 27C shows cytokine induction results for monkeys treated with CLY-023 at 0.1 mg/kg, iv. FIG. 27D shows cytokine induction results for monkeys treated with CLY-023 at 1 mg/kg, iv. Of note is that no in life observations or signs of cytokine release syndrome in monkeys dosed up to 1 mg/kg IV were detected. FIG. 27E shows the plasma concentration of CLY-024 for monkeys treated with CLY-024 at various concentrations. FIG. 27F shows CD20+ B cell count in blood of monkeys treated with CLY-024. Of note is that moderate B cell depletion observed in monkey dosed at 1 mg/kg IV. Minimal B cell depletion observed at lower doses (<1 mg/kg IV). FIG. 27G shows cytokine induction results for monkeys treated with CLY-024 at 0.01 mg/kg i.v. FIG. 27H shows cytokine induction results for monkeys treated with CLY-024 at 0.1 mg/kg i.v. FIG. 27I shows cytokine induction results for monkeys treated with CLY-024 at 1 mg/kg i.v. No in life observations or signs of cytokine release syndrome in cynomolgus monkeys dosed up to 1 mg/kg IV. FIG. 27J shows plasma concentration of CLY-020 in treated monkeys. FIG. 27K shows CD20+ B cell count in blood of monkeys treated with CLY-020 over 28 days. FIG. 27L shows cytokine induction in CLY-020-treated monkeys for IL-2, IFN gamma, IL-6 and TNF. FIG. 27M shows plasma concentration of CLY-018 in treated monkeys. FIG. 27N shows CD20+ B cell count in monkeys treated with CLY-018. FIG. 27O shows cytokine induction for monkeys treated with CLY-018 at a concentration of 0.1 mg/kg. FIG. 27P shows cytokine induction for monkeys treated with CLY-018 at 1 mg/kg. FIG. 27Q shows cytokine induction for monkeys treated with CLY-018 at 2.5 mg/kg. FIG. 27R shows cytokine induction for monkeys treated with CLY-018 at 9 mg/kg. No in life observations or signs of cytokine release syndrome in cynomolgus monkeys dosed up to 9 mg/kg IV was observed.

FIGS. 28A-28T show activity of the CLY-018 construct in healthy human PBMCs and PBMCs of autoimmune subjects (e.g., RA, SLE, and MG). FIG. 28A shows average B-cell fold change/killing of healthy human PBMC treated with CLY-018 over the course of 7 days. FIG. 28B shows average CD4+ T cell fold change of healthy human PBMCs treated with CLY-018. FIG. 28C shows average CD8+ T cell fold change of healthy human PBMC treated with CLY-018 over the course of 7 days. FIG. 28D shows average T reg fold change of healthy human PBMC treated with CLY-018 over the course of 7 days. FIG. 28E shows results of CD8+ T cell expansion for the Tn, Tscm, Ttm, Tcm, Tem, Teff, and Temra subsets of healthy human PBMCs incubated with CLY-018. FIG. 28F shows average B-cell fold change/killing of PBMC from subjects with RA treated with CLY-018 over 7 days. FIG. 28G shows average CD4+ T cell fold change of PBMC from subjects with RA treated with CLY-018 over 7 days. FIG. 28H shows average CD8+ T cell fold change of PBMC from subjects with RA treated with CLY-018. FIG. 28I shows average T reg fold change of PBMC from subjects with RA treated with CLY-018. FIG. 28J shows results of CD8+ T cell expansion for the Tn, Tscm, Ttm, Tcm, Tem, Teff, and Temra subsets of the RA subjects PBMCs incubated with CLY-018. FIG. 28K shows average B-cell fold change/killing of PBMC from 3 SLE subjects treated with CLY-018 over 7 days. FIG. 28L shows average CD4+ T cell fold change of PBMC from 3 SLE subjects treated with CLY-018 over 7 days. FIG. 28M shows average CD8+ T cell fold change of PBMC from 3 SLE subjects treated with CLY-018. FIG. 28N shows average T reg fold change of PBMC from 3 SLE subjects treated with CLY-018. FIG. 28O shows results of CD8+ T cell expansion for the Tn, Tscm, Ttm, Tcm, Tem, Teff, and Temra subsets of the SLE subjects PBMCs incubated with CLY-018. FIG. 28P shows average B-cell fold change of PBMC from 3 MG subjects treated with CLY-018 over 7 days. FIG. 28Q shows average CD4+ T cell fold change of PBMC from 3 MG subjects treated with CLY-018 over 7 days. FIG. 28R shows average CD8+ T cell fold change of PBMC from 3 MG subjects treated with CLY-018. FIG. 28S shows average T reg fold change of PBMC from 3 MG subjects treated with CLY-018. FIG. 28T shows results of CD8+ T cell expansion for the Tn, Tscm, Ttm, Tcm, Tem, Teff, and Temra subsets of the MG subjects PBMCs incubated with CLY-018.

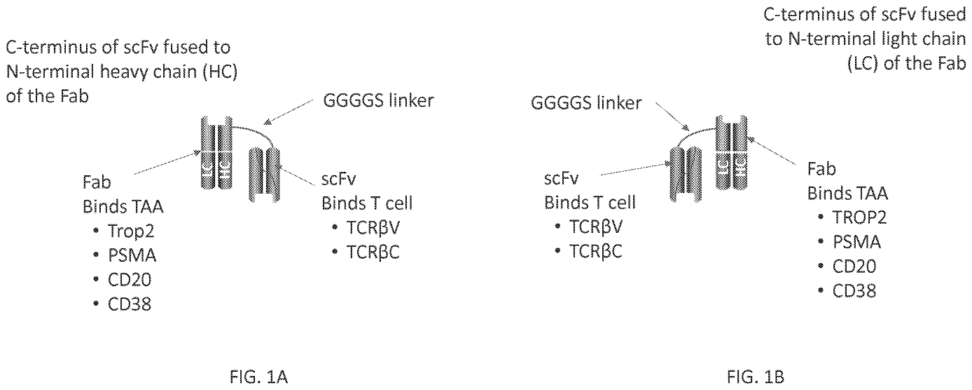

### DETAILED DESCRIPTION

### Definitions

Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa unless the content clearly dictates otherwise. In the event that any description of a term set forth conflicts with any document incorporated herein by reference, the description of the term set forth below shall control.

The terms “a”, “an”, and “the”, as used herein, include plural references unless the context clearly dictates otherwise.

The term “about” or “approximately”, as used herein, in reference to a number or range of numbers, is understood to mean the stated number and numbers+/−10% thereof, or 10% below the lower listed limit and 10% above the higher listed limit for the values listed for a range (except where such number would exceed 100% of a possible value or go below 0%). It is used to indicate that a value includes the inherent variation of error for the device, or the method being employed to determine the value, or the variation that exists among the samples being measured.

The term “between”, as used in a phrase as such “between A and B” or “between A-B” refers to a range including both A and B.

The terms “comprise” and its grammatical equivalents, as used herein, specify the presence of stated features, integers, steps, operations, elements, and/or components, but do not preclude the presence or addition of one or more other features, integers, steps, operations, elements, components, and/or groups thereof.

The terms “including”, “includes”, “included”, and other forms, as used herein, are not limiting.

The terms “or” and “and/or”, as used herein, include any, and all, combinations of one or more of the associated listed items.

The phrase “any range or subrange therebetween” or equivalents, are used herein to denote the intention that disclosure of any range or series of possible values, inherently also discloses all ranges and subranges encompassed by the highest and lowest values disclosed. This term includes the entire range from highest to lowest disclosed values, as well as subranges from any two or more disclosed points. This term is also intended to disclose any subranges encompassed anywhere within the highest and lowest disclosed values, including between two points that are explicitly recited in the document, up to one decimal point. Thus, disclosure of values 0, 5, 10, 15, 20, including all ranges and subranges therebetween, should be interpreted as also encompassing a range from 0-20, a range from 0-5 or 5-15, as well as a range from 2-16, or 3.1 to 19.8, etc. Unless otherwise indicated, it is to be understood that all numbers expressing quantities, ratios, and numerical properties of ingredients, reaction conditions, and so forth, used in the specification are contemplated to be able to be modified in all instances by the term “including all ranges and subranges therebetween”.

In the present description, any concentration range, percentage range, ratio range, or integer range is to be understood to include the value of any integer within the recited range and, when appropriate, fractions thereof (such as one tenth and one hundredth of an integer), unless otherwise indicated. For example, disclosure of a range of 1 to 10 mM concentration encompasses values of 2, 3, 6, 7, and 9 mM, as well as 1.3, 2.5, and 7.6 mM concentrations.

The term “polynucleotide” or “nucleic acid”, as used herein, refers to deoxyribonucleic acid (DNA), ribonucleic acid (RNA) and DNA/RNA hybrids. Polynucleotides may be single-stranded or double-stranded and either recombinant, synthetic, or isolated. Polynucleotides include but are not limited to: pre-messenger RNA (pre-mRNA), messenger RNA (mRNA), RNA, genomic DNA (gDNA), PCR amplified DNA, complementary DNA (cDNA), synthetic DNA, or recombinant DNA. Polynucleotides can comprise modified nucleotides or bases, and/or their analogs, or any substrate that can be incorporated into a polymer by DNA or RNA polymerase or by a synthetic reaction. Unless specified otherwise, the left-hand end of any single-stranded polynucleotide sequence disclosed herein is the 5′ end; the left-hand direction of double-stranded polynucleotide sequences is referred to as the 5′ direction. The direction of 5′ to 3′ addition of nascent RNA transcripts is referred to as the transcription direction.

The term “coding sequence” or a polynucleotide which “encodes” a polypeptide or polypeptide molecule, as used herein, is a nucleic acid molecule which is transcribed (in the case of DNA) and translated (in the case of mRNA) into a polypeptide or polypeptide molecule when placed under the control of appropriate regulatory sequences. The boundaries of the coding sequence are determined by a start codon at the 5′ (amino) terminus and a translation stop codon at the 3′ (carboxy) terminus. A transcription termination sequence may be located 3′ to the coding sequence.

The terms “polypeptide” and “peptide” and “protein”, as used herein, refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art.

The terms “amino-terminal” and “carboxyl-terminal” are used herein to denote positions within the polypeptide molecules described herein. Where the context allows, these terms are used with reference to a particular sequence or portion of a polypeptide molecule to denote proximity or relative position.

The term “disulfide bond”, as used herein, refers to the covalent linkage between the thiol groups of two cysteine residues. The specific position/location of the disulfide bond can be defined using the location of the two cysteine residues within a protein or polypeptide. For instance, a VH44-VL100 disulfide bond in a TCRβV-binding domain of a polypeptide molecule refers to a disulfide bond formed between the cysteine residue at amino acid position 44 of the VH region of the TCRβV-binding domain and the cysteine residue at amino acid position 100 of the VL region of the TCRβV-binding domain. Similarly, a VH100B-VL49 disulfide bond of a TCRβV-binding domain of a polypeptide molecule refers to a disulfide bond formed between the cysteine residue at amino acid position 100B of the VH region of the TCRβV-binding domain and the cysteine residue at amino acid position 49 of the VL region of the TCRβV-binding domain. It is well known to a skilled artisan that antibodies have different HCDR3 lengths and additional residues may occur between positions VH100 and VH101 that can be numbered with a letter from A to K according to the Kabat numbering scheme (e.g., VH100A, VH100B, VH100C . . . H100K).

The term “peptide linker”, as used herein, generally refers to a short polypeptide sequence connecting two sub-domains of a polypeptide. Non-limiting examples of peptide linkers include flexible linkers comprising glycine-serine repeats, and peptide linkers derived from (a) an interdomain region of a transmembrane protein (e.g., a type I transmembrane protein); (b) a stalk region of a type II C-lectin; or (c) an immunoglobulin hinge. In some embodiments, a linker provides a spacer function compatible with interaction of the two sub-binding domains so that the resulting polypeptide retains a specific binding affinity to the same target molecule as an antibody that comprises the same light and heavy chain variable regions. In some embodiments, the linker connects two or more binding domains (e.g., the TCRβV-binding domain and the CD19-binding domain). In some embodiments, the linker connects a binding domain with a half-life extension moiety. In some embodiments, the linker connects a VH region of an scFv and a VL region of an scFv. In some embodiments, the linker connects a VH region of an scFv and a VL region of an scFv.

The term “variant”, when used in relation to a polypeptide, refers to a polypeptide comprising one or more (such as, for example, about 1 to about 25, about 1 to about 20, about 1 to about 15, about 1 to about 10, or about 1 to about 5) amino acid sequence substitutions, deletions, and/or insertions as compared to a wildtype or parental amino acid sequence. Variants may be naturally occurring, such as allelic or splice variants, or may be artificially constructed. Polypeptide variants may be prepared from the corresponding nucleic acid molecules encoding the variants.

The term “antigen,” as used herein, refers to a structure to which an antibody or an antibody fragment can specifically bind. An antigen may be a polypeptide, carbohydrate, nucleic acid, lipid, hapten, or other naturally occurring or synthetic compound.

The term “antigen-binding domain,” as used herein, refers to a portion of a polypeptide molecule that specifically binds a target antigen or target epitope. Antigen-binding domains may comprise antibodies or antigen-binding fragments thereof.

The term “antigen-binding fragment,” as used herein, refers to a portion of an antibody heavy and/or light chain polypeptide that retains some or all of the binding activity of the antibody from which the fragment was derived. An antigen-binding fragment may comprise 1, 2, 3, 4, 5, or all 6 CDRs of a variable heavy chain (VH) region and/or variable light chain (VL) region. Non-limiting examples of functional fragments include single-chain Fvs (scFv), Fab fragments, CrossFab fragments, F(ab′) fragments, F(ab)₂ fragments, F(ab′)2 fragments, disulfide-linked Fvs (dsFv), Fd fragments, Fv fragments, diabody, triabody, tetrabody, and minibody. Such functional antigen-binding fragments can be found in, for example, Harlow and Lane, Antibodies: A Laboratory Manual (1989); Mol. Biology and Biotechnology: A Comprehensive Desk Reference (Myers, ed., 1995); Huston, et al, 1993, Cell Biophysics 22:189-224; Pluckthun and Skerra, 1989, Meth. Enzymol. 178:497-515; and Day, Advanced Immunochemistry (2d ed. 1990).

The term “antibody,” “immunoglobulin,” or “Ig” is used interchangeably herein, and is used in the broadest sense and specifically covers, for example, monoclonal antibodies (including agonist, antagonist, neutralizing antibodies, full length or intact monoclonal antibodies), antibody compositions with polyepitopic or monoepitopic specificity, polyclonal or monovalent antibodies, multivalent antibodies, and multi-specific antibodies (e.g., bispecific antibodies). A conventional antibody is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck, ed., 2d ed. 1995); and Kuby, Immunology (3d ed. 1997). An antibody can be human, humanized, chimeric and/or affinity matured, as well as an antibody from a different species, for example, mouse, rabbit, etc. Antibodies also include, but are not limited to, synthetic antibodies, recombinantly produced antibodies, camelized antibodies or their humanized variants, and intrabodies. An antibody can be selected from any class of immunoglobulins, including IgM, IgG, IgD, IgA and IgE, and any isotype, including IgG1, IgG2, IgG3 and IgG4 (e.g., variants of IgG4 and IgG4 nullbody). An antibody can comprise kappa or lambda light chain constant sequences.

The term “Fab” or “Fab region”, as used herein, refers to an antibody region that binds to an antigen. A conventional IgG usually comprises two Fab regions, each residing on one of the two arms of the Y-shaped IgG structure. Each Fab region is typically composed of one variable region and one constant region of each of the heavy and the light chain. More specifically, the variable region and the constant region of the heavy chain in a Fab region are VH and CH1 regions, and the variable region and the constant region of the light chain in a Fab region are VL and CL regions. The VH, CH1, VL, and CL in a Fab region can be arranged in various ways to confer an antigen binding capability. For example, VH and CH1 regions can be on one polypeptide, and VL and CL regions can be on a separate polypeptide, similarly to a Fab region of a conventional IgG. Alternatively, VH, CH1, VL and CL regions can all be on the same polypeptide and oriented in different orders.

The term “single-chain variable fragment (scFv)”, as used herein, refers to a fusion protein of the variable heavy chain (VH) and the variable light chain (VL) of an antibody, connected with a short scFv linker peptide. In some embodiments, the scFv linker is about 5 amino acids to about 30 amino acids in length. In some embodiments, the scFv linker is rich in glycine for flexibility, as well as serine or threonine for solubility, and can either connect the N-terminus of the VH region with the C-terminus of the VL region, or vice versa. scFvs retain the specificity of the original antibody, despite removal of the constant regions and the introduction of the scFv linker. scFvs are, described in e.g., Houston, J. S., Methods in Enzymol. 203 (1991) 46-96).

The term “heavy chain”, when used in reference to an antibody, refers to a polypeptide chain of about 50-70 kDa, wherein the amino-terminal portion includes a variable region of about 120 to 130 or more amino acids, and a carboxy-terminal portion includes a constant region. The constant region can be one of five distinct types, (e.g, isotypes) referred to as alpha, delta, epsilon, gamma, and mu, based on the amino acid sequence of the heavy chain constant region. The distinct heavy chains differ in size: alpha, delta, and gamma contain approximately 450 amino acids, while epsilon and mu contain approximately 550 amino acids. When combined with a light chain, these distinct types of heavy chains give rise to five well known classes (e.g., isotypes) of antibodies, IgA, IgD, IgE, IgG, and IgM, respectively, including four subclasses of IgG, namely IgG1, IgG2, IgG3, and IgG4.

The term “light chain”, when used in reference to an antibody, refers to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, referred to as kappa or lambda based on the amino acid sequence of the constant domains.

The term “constant region” or “constant domain,” as used herein, refers to a carboxy terminal portion of the light and heavy chain which is not directly involved in binding of the antibody to antigen but exhibits various effector function, such as interaction with the Fc receptor. This portion has a more conserved amino acid sequence relative to the variable region. The constant region may contain the CH1, CH2, and CH3 regions of the heavy chain and the CL region of the light chain.

The term “variable region”, “variable domain”, “V region”, or “V domain”, as used herein, refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” or “complementarity determining regions” that are each about 9-12 amino acids long. The variable regions of heavy and light chains each comprise four FRs, largely adopting a β sheet configuration, connected by three hypervariable regions, which form loops connecting, and in some cases form part of, the β sheet structure. The hypervariable regions in each chain are held together in close proximity by the FRs and, with the hypervariable regions from the other chain, contribute to the formation of the antigen-binding site of antibodies (see, e.g., Kabat et al, Sequences of Proteins of Immunological Interest (5th ed. 1991)).

The complementarity determining regions (CDRs) have been defined by well-known numbering systems. For example, the Kabat Complementarity Determining Regions (CDRs) are based on sequence variability and are the most commonly used (see, e.g., Kabat, et al., supra). Chothia refers instead to the location of the structural loops (see, e.g., Chothia and Lesk, J. Mol. Biol., 1987, 196:901-17). The end of the Chothia CDR-H1 loop when numbered using the Kabat numbering convention varies between H32 and H34 depending on the length of the loop (this is because the Kabat numbering scheme places insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Rontermann and Diibel, eds., 2d ed. 2010)). The “contact” CDRs are based on an analysis of the available complex crystal structures. Another universal numbering system that has been developed and widely adopted is ImMunoGeneTics (IMGT) Information System® (Lafranc, et al, Dev. Comp. Immunol., 2003, 27(1):55-77). IMGT is an integrated information system specializing in immunoglobulins (IG), T-cell receptors (TCR), and major histocompatibility complex (MHC) of human and other vertebrates. An additional numbering system (AHon) has been developed by Honegger and Pluckthun, J. Mol. Biol., 2001, 309: 657-70. Correspondence between the numbering system, including, for example, the Kabat numbering and the IMGT unique numbering system, is well known to one skilled in the art (see, e.g., Kabat, supra, Chothia and Lesk, supra; Martin, supra, Lefranc, et al., supra).

The boundaries of a given CDR may vary depending on the scheme used for identification. Thus, unless otherwise specified, the CDRs of a given antibody or region thereof, such as a variable region, should be understood to encompass the complementary determining region as defined by any of the known schemes described herein. In some instances, the scheme for identification of a particular CDR or CDRs is specified, such as the CDR as defined by the Kabat, Chothia, or Contact method. In other cases, the particular amino acid sequence of a CDR is given. In some embodiments, a combination of CDR numbering systems may be used. In such embodiments, CDR sequences of a given binding domain or polypeptide molecule as determined by multiple numbering systems (e.g., a CDR1 sequence as determined by the Kabat, IMGT, and Chothia numbering systems) are compiled into a single sequence that encompasses the entirety of each of the CDR amino acid ranges in the variable region.

As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody.

The term “polypeptide molecule,” as used herein, refers to a polypeptide or complex of polypeptides comprising at least one binding domain that specifically binds to a target. In some embodiments, the polypeptide molecule is a multi-specific polypeptide molecule. As used herein, a “multi-specific polypeptide molecule” refers to a polypeptide comprising two or more binding domains each capable of specifically binding to a target. For example, the polypeptide molecules described herein may comprise 2, 3, 4, or more binding domains and may be able to bind 2, 3, 4, or more targets. In some embodiments, the polypeptide molecule comprises a first binding domain that specifically binds to a T cell receptor beta variable region (TCRβV) or a T cell receptor beta constant region (TCRβC) expressed on a T cell. In a further embodiment, the polypeptide molecule comprises a second binding domain specifically binding to a tumor-associated antigen (TAA), such as CD19 or PSMA.

The term “vector”, as used herein, refers to a substance that is used to carry or introduce a nucleic acid sequence (e.g., a nucleic acid sequence encoding polypeptide molecule as described herein) into a host cell. Vectors applicable for use include, for example, plasmids, phage vectors, viral vectors, episomes, and artificial chromosomes. A vector may include sequences that direct autonomous replication in a cell, or may include sequences sufficient to allow integration into host cell DNA. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like, which are well known in the art. When two or more nucleic acid molecules are to be co-expressed (e.g., both an antibody heavy and light chain or an antibody VH and VL), both nucleic acid molecules can be inserted, for example, into a single expression vector or in separate expression vectors. For single vector expression, the encoding nucleic acids can be operationally linked to one common expression control sequence or linked to different expression control sequences. The introduction of nucleic acid molecules into a host cell can be confirmed using methods well known in the art. Such methods include, for example, nucleic acid analysis such as Northern blots or polymerase chain reaction (PCR) amplification of mRNA, immunoblotting for expression of gene products, or other suitable analytical methods to test the expression of an introduced nucleic acid sequence or its corresponding gene product. It is understood by those skilled in the art that the nucleic acid molecules are expressed in a sufficient amount to produce a desired product and it is further understood that expression levels can be optimized to obtain sufficient expression using methods well known in the art.

The term “binds” or “binding,” as used herein, refers to a covalent or non-covalent interaction between molecules (e.g., forming a complex by interactions). Exemplary non-covalent interactions include hydrogen bonds, ionic bonds, hydrophobic interactions, and/or van der Waals interactions. Terms such as “specific binding,” “specifically binds to,” or “is specific for” means that a binding domain or a polypeptide molecule comprising a binding domain binds the target with an affinity or Kₐ (i.e., an equilibrium association constant of a particular binding interaction with units of 1/M) equal to or greater than 10⁵ M⁻¹, while not significantly binding other components present in a test sample.

The term “binding affinity,” as used herein, refers to the strength of the sum total of noncovalent interactions between a binding domain X and its binding partner Y. Unless indicated otherwise, as used herein, “binding affinity” refers to intrinsic binding affinity which reflects a 1:1 interaction between members of a binding pair (e.g., antibody and antigen). Binding domains can be classified as “high affinity” binding domains and “low affinity” binding domains. “High affinity” binding domains refer to those binding domains with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. “Low affinity” binding domains refer to those binding domains with a Kₐ of up to 107 M⁻¹, up to 10⁶ M⁻¹, up to 10⁵ M⁻¹. High affinity binding domains generally bind the binding partners faster and tend to remain bound longer, whereas low affinity binding domains generally bind the binding partners slowly and tend to dissociate readily. Affinity can also be defined as an equilibrium dissociation constant (K_{d}) of a particular binding interaction with units of M (e.g., 10⁻⁵ M to 10⁻¹³, or about 1000 nM, about 900 nM, about 800 nM, about 700 nM, about 600 nM, about 500 nM, about 300 nM, about 250 nM, about 200 nM, about 150 nM, about 100 nM, about 50 nM, about 25 nM, about 10 nM, or about 5 nM). Affinities of binding domains according to the present disclosure can be readily determined using conventional techniques (see, e.g., Scatchard et al. (1949) Ann. N.Y. Acad. Sci. 51:660; and U.S. Pat. Nos. 5,283,173, 5,468,614, or the equivalent).

The term “cytokine”, as used herein, refers to small proteins or variants thereof that act as signaling molecules to regulate immunity, inflammation, and hematopoiesis. Cytokines include chemokines, interferons, interleukins, lymphokines, and tumor necrosis factors. Specific examples of cytokines include interleukin-2 (IL-2), interleukin-7 (IL-7), interleukin-10 (IL-10), interleukin-12 (IL-12), interleukin-15 (IL-15), interleukin-18 (IL-18), interleukin-21 (IL-21), interferon gamma (IFN-7), or combinations or variants thereof.

The term “subject”, as used herein, refers to an “animal” and in particular a “mammal” such as a non-primate (e.g., mice, rats, bovines, horses, household cats, tigers and other large cats, dogs, pigs, rabbits, goats, deer, sheep, ferrets, gerbils, guinea pigs, hamsters, bats, and birds (e.g., chickens, turkeys, and ducks)) or a primate (e.g., monkeys, baboons, chimpanzees, and human). The term may be used interchangeably with the term “patient” or “individual”. In some embodiments, the subject is a mammal, e.g., a human, diagnosed with a disease or disorder provided herein. In some embodiments, the subject is a mammal, e.g., a human, at risk of developing a disease, disorder, or condition described herein. In some embodiments, the subject is human. The term does not denote a particular age or sex. Thus, individuals of all ages, from newborn to adult, whether male or female, are intended to be covered.

The term “administer”, “administration”, or “administering,” as used herein refers to the act of injecting or otherwise physically delivering a substance (e.g., a polypeptide molecule or pharmaceutical composition thereof) to a subject (e.g., human), such as by oral, mucosal, topical, intradermal, parenteral, intravenous, intravitreal, intraarticular, subretinal, intramuscular, intrathecal delivery and/or any other method of physical delivery described herein or known in the art. The delivery can be systemic or to a specific tissue.

The term “effective amount”, as used herein, refers to an amount of a therapeutic (e.g., a pharmaceutical composition provided herein) which is sufficient to prevent, diagnose, treat, delay the onset of, reduce and/or ameliorate the severity, duration, advancement, and/or recurrence of a given condition, disorder or disease and/or a symptom related thereto. The term also encompasses an amount necessary to improve or enhance the prophylactic or therapeutic effect (s) of another therapy or to serve as a bridge to another therapy.

The term “prevent”, as used herein, refers to a pharmaceutical or other intervention regimen for reducing the likelihood of the onset (or recurrence) of a disease, disorder, condition, or associated symptom(s).

The term “population” of cells, as used herein, refers to any number of cells greater than 1, for example, at least about 1×10³ cells, at least about 1×10⁴ cells, at least about 1×10⁵ cells, at least about 1×10⁶ cells, at least about 1×10⁷ cells, at least about 1×10⁸ cells, at least about 1×10⁹ cells, at least about 1×10¹⁰ cells, at least about 1×10¹¹ or more cells. A population of cells may refer to an in vitro population (e.g., a population of cells in culture) or an in vivo population (e.g., a population of cells residing in a particular tissue).

The term “effector T cell”, as used herein, refers to active form of T cells that have relatively short lifespans and carry out the functions of an immune response. Effector T cells can be cytotoxic (CD8+), helper (CD4+, non-reg), and regulatory T cells. Specific types of effector T cells include effector memory T cells (T_{EM}), T effector cells (T_{EFF}), and T effector cells re-expressing CD45RA (T_{EMRA}). In some embodiments, the effector T cell is TCRβV+. In some embodiments, the effector T cell is TCRαV+.

The term “memory T cell”, as used herein, refers to long-lived T cells that remain in the body after a pathogen has been cleared. Memory T cells are highly capable of responding to antigens upon reintroduction. Specific types of memory T cells include central memory T cells (T_{CM}), T memory stem cells (T_{SCM}), and transitional memory T cells (TTM).

The term “cytotoxicity”, as used herein, refers to an ability of an effector cell to kill a target cell. In some embodiments, the effector cell is a TCRβV+ T cell. In some embodiments, the effector cell is a TCRαV+ T cell. In some embodiments, the target cell is a B cell, such as a CD19+ B cell. In some embodiments, the target cell is a PSMA+ cell.

The term “durability”, as used herein, refers to the ability of a T cell, or population of T cells, to maintain their effector function (i.e., ability to kill a target cell) and/or the capacity of a T cell to expand and differentiate in a subject or sample.

The term “contact”, as used in the context of contacting a target cell with a polypeptide molecule described herein, is intended to include incubating the target cell and the polypeptide molecule together in vitro, or administering the polypeptide molecule to a subject where the polypeptide molecule “contacts” the target cell in vivo.

The term “pharmaceutical composition” or “therapeutic composition”, as used herein, refers to a composition capable of being administered to a subject for the treatment of a particular disease or disorder.

The term “pharmaceutically acceptable excipient, carrier or diluent,” as used herein, refers to any substance formulated alongside the active ingredient (i.e., the polypeptide molecule) of a pharmaceutical composition that allows the active ingredient to retain biological activity and is non-reactive with the subject's immune system. Such a substance can be included for the purpose of long-term stabilization, bulking up solid formulations that contain potent active ingredients in small amounts, or to confer a therapeutic enhancement on the active ingredient in the final dosage form, such as facilitating absorption, reducing viscosity, or enhancing solubility. The selection of appropriate substance can depend upon the route of administration and the dosage form, as well as the active ingredient and other factors. Compositions having such substances can be formulated by well-known conventional methods (see, e.g., Remington, The Science and Practice of Pharmacy, 23rd edition, A. Adejare, ed., Academic Press, 2020).

As used herein, the term “sequence identity” refers to the extent to which two optimally aligned polynucleotides or polypeptide sequences are invariant throughout a window of alignment of residues, e.g., nucleotides or amino acids. An “identity fraction” for aligned segments of a test sequence and a reference sequence is the number of identical residues which are shared by the two aligned sequences divided by the total number of residues in the reference sequence segment, i.e., the entire reference sequence or a smaller defined part of the reference sequence. “Percent identity” is the identity fraction times 100. Comparison of sequences to determine percent identity can be accomplished by a number of well-known methods, including for example by using mathematical algorithms, such as, for example, those in the BLAST suite of sequence analysis programs. Unless noted otherwise, the term “sequence identity” in the claims refers to sequence identity as calculated by Clustal Omega® using default parameters.

General methods in molecular and cellular biochemistry can be found in such standard textbooks as Molecular Cloning: A Laboratory Manual, 3rd Ed. (Sambrook etal., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

### Polypeptide Molecules

In some embodiments, the present disclosure provides a polypeptide molecule comprising at least one binding domain. In some embodiments, the polypeptide molecules comprises 1, 2, 3, 4, 5, 6, 7, 8, 9, or 10 binding domains, including any ranges and subranges therebetween. In some embodiments the polypeptide molecule of the present disclosure comprises a first binding domain is a T cell binding domain and a second binding domain that is a is a tumor-associated antigen (TAA) binding domain. In some embodiments the polypeptide molecule comprises a T cell binding domain, a TAA binding domain, and a half-life extending moiety. In some embodiments, the half-life extending moiety comprises a binding domain (such as an albumin binding domain). In some embodiments, the polypeptide molecule comprises linkers connecting the various binding domains.

In some embodiments, any one of the binding domains within the polypeptide molecule comprises a Fab. In some embodiments, any one of the binding domains within the polypeptide molecule is an scFv. In some embodiments, the T cell-binding domain is a TCRβV-binding domain (e.g., a TCRβV6-5-binding domain or a TCRβV12-3-binding domain). In some embodiments, the T cell-binding domain is a TCRβC-binding domain (e.g., a TCRβC1-binding domain or a TCRβC2-binding domain). In some embodiments, the binding domain is a tumor-associated antigen (TAA) binding domain (e.g., a PSMA-binding domain or a CD19-binding domain).

In some embodiments, the polypeptide molecule of the present disclosure comprises at least one binding domain. In some embodiments, any one of the binding domains comprise an immunoglobulin molecule. In some embodiments, the immunoglobulin molecule is an antibody or antibody fragment. In some embodiments, any one of the binding domains is a full-length antibody, e.g., a polyclonal or monoclonal antibody. In some embodiments, any one of the binding domains is an antibody fragment, such as, e.g., a single domain antibody (sdAb, also referred to as a VHH antibody or nanobody), single-chain variable fragment (scFv), Fe, scFv-Fc, Fv, Fab, Fab′, or F(ab′)₂. In some embodiments, any one of the binding domains is a chimeric antibody. In some embodiments, the binding domain is a humanized antibody. In some embodiments, the binding domain is a T cell-binding domain. In some embodiments, the T cell-binding domain is a TCRβV-binding domain. In some embodiments, the T cell-binding domain is a TCRβC-binding domain. In some embodiments, the binding domain is a TAA-binding domain. In some embodiments, the TAA-binding domain is a CD19-binding domain.

In some embodiments, the polypeptide molecule comprises at least one binding domain and the binding domain comprises a heavy chain. In some embodiments, the heavy chain is an IgA, IgD, IgE, IgG, or IgM heavy chain. In some embodiments, the heavy chain is a subclass of IgA, IgD, IgE, IgG, or IgM, such as, e.g., IgG₁, IgG₂, IgG₃, IgG₄, IgA₁, and IgA₂. In some embodiments, the binding domain comprises an IgG1 heavy chain. In some embodiments, the binding domain comprises an IgG4 heavy chain.

In some embodiments, the polypeptide molecule comprises an immunoglobulin constant region. The immunoglobulin constant region or a portion thereof may be obtained from a number of different sources. In some embodiments, an immunoglobulin constant region or a portion thereof is derived from a human immunoglobulin. In some embodiments, an immunoglobulin constant region or a portion thereof is derived from an immunoglobulin of another mammalian species, including for example, a rodent (e.g., a mouse, rat, rabbit, guinea pig) or non-human primate (e.g., chimpanzee, macaque) species. Moreover, the immunoglobulin constant region or a portion thereof may be derived from any immunoglobulin class, including IgM, IgG, IgD, IgA and IgE, and any immunoglobulin isotype, including IgG1, IgG2, IgG3 and IgG4. In some embodiments, the immunoglobulin constant region is derived from human isotype IgG1.

A variety of the immunoglobulin constant region gene sequences (e.g., human constant region gene sequences) are available in the form of publicly accessible deposits. Constant region domains sequence can be selected having a particular effector function (or lacking a particular effector function) or with a particular modification to reduce immunogenicity. Many sequences of antibodies and antibody-encoding genes have been published and suitable Ig constant region sequences (e.g., hinge, CH2, and/or CH3 sequences, or portions thereof) can be derived from these sequences using art recognized techniques. The genetic material obtained using any of the foregoing methods may then be altered or synthesized to obtain polypeptides of the present disclosure. It will further be appreciated that the scope of this disclosure encompasses alleles, variants and mutations of constant region DNA sequences.

In some embodiments, the polypeptide molecule comprises an immunoglobulin constant region or a portion thereof. An immunoglobulin constant region is comprised of domains denoted CH (constant heavy) domains (CH1, CH2, etc.). Depending on the isotype, (i.e., IgG, IgM, IgA IgD, or IgE), the constant region can be comprised of three or four CH domains. Some isotypes (e.g., IgG) constant regions also contain a hinge region. See Janeway et al. 2001, Immunobiology, Garland Publishing, N.Y., N.Y.

In some embodiments, the immunoglobulin constant region includes all domains and the hinge region or portions thereof. In some embodiments, the immunoglobulin constant region or a portion thereof comprises a CH2 domain, a CH3 domain, and a hinge region, i.e., an Fc region or an FcRn binding partner.

In some embodiments, the polypeptide molecule comprises an Fc region. In some embodiments, the Fc region may comprise one or more of the CH2, CH3, and CH4 domains, as well as the hinge region. In some embodiments, fusion proteins comprising an Fc region of an immunoglobulin bestow several desirable properties on a fusion protein including increased stability, increased serum half-life (see Capon et al., 1989, Nature 337:525) as well as binding to Fc receptors such as the neonatal Fc receptor (FcRn) (U.S. Pat. Nos. 6,086,875, 6,485,726, 6,030,613; WO 03/077834; US2003-0235536A1).

In some embodiments, the Fc region of the polypeptide molecule comprises two polypeptide chains. In some embodiments, each polypeptide chain is derived from an antibody heavy chain. In some embodiments, both polypeptide chains of the Fc region have the same domain organization (e.g., comprising a CH2 domain and a CH3 domain).

In some embodiments, the Fc region of the polypeptide molecule comprises a CH2 domain (about amino acids 231-340 of an antibody Fc region according to EU numbering), and a CH3 domain (about amino acids 341-438 of an antibody Fc region according to EU numbering). In some embodiment, the Fc region comprises a hinge (e.g., upper, middle, and/or lower hinge region) domain (about amino acids 216-230 of an antibody Fc region according to EU numbering), a CH2 domain, and a CH3 domain. In some embodiments, the Fc region comprises a hinge, a CH2 domain, a CH3 domain, and a CH4 domain. In some embodiments, the Fc region comprises at least one of: a hinge, a CH2 domain, a CH3 domain, and a CH4 domain, or a variant, portion, or fragment thereof. In some embodiments, the Fc region comprises a hinge domain (or a portion thereof) fused to a CH3 domain (or a portion thereof), a hinge domain (or a portion thereof) fused to a CH2 domain (or a portion thereof), a CH2 domain (or a portion thereof) fused to a CH3 domain (or a portion thereof), or a CH2 domain (or a portion thereof) fused to both a hinge domain (or a portion thereof) and a CH3 domain (or a portion thereof). In some embodiments, the Fc region lacks at least a portion of a CH2 domain (e.g., all or part of a CH2 domain). In some embodiments, the Fc region comprises or consists of amino acids corresponding to EU numbers 221 to 447.

In some embodiments, the Fc region of the polypeptide molecule is derived from a human immunoglobulin. In some embodiments, an Fc region is derived from an immunoglobulin of another mammalian species, including for example, a rodent (e.g., a mouse, rat, rabbit, guinea pig) or non-human primate (e.g., chimpanzee, macaque) species. In some embodiments, the Fc region is derived from any immunoglobulin class, including IgM, IgG, IgD, IgA and IgE, and any immunoglobulin isotype, including IgG1, IgG2, IgG3 and IgG4. In some embodiments, it is derived from the human isotype IgG1.

In some embodiments, the polypeptide molecule of the present disclosure is a monospecific antibody, F(ab′)2, F(ab′)3, a bispecific antibody, a CrossFab′, a trispecific antibody, a minibody, a diabody, a bispecific-scFv, a triabody, or a tetrabody. In some embodiments, the polypeptide molecule is a bispecific polypeptide molecule. In some embodiments, the polypeptide molecule is a full length antibody or antibody fragment that binds more than one antigen. In some embodiments, the polypeptide molecule is a full length antibody or antibody fragment that binds two antigens.

In some embodiments, the polypeptide molecule of the present disclosure comprises at least one binding domain. In some embodiments, the binding domain is an scFv. In some embodiments, the C-terminus of the VH region is linked to the N-terminus of the VL region. In some embodiments, the C-terminus of the VL region is linked to the N-terminus of the VH region. In some embodiments, the VH region and the VL region are linked by a peptide linker. In some embodiments, the peptide linker comprises the formula of (Gly4Ser)ₙ, wherein n=1-5 (SEQ ID NO: 1424). In some embodiments, the peptide linker is (Gly4Ser)₃ (SEQ ID NO: 1342). In some embodiments, the peptide linker is (Gly4Ser)₁ (SEQ ID NO: 1425).

In some embodiments, the polypeptide molecule of the present disclosure comprises at least one binding domain. In some embodiments, the binding domain comprises a Fab comprising a VH region, a VL region, a first heavy chain constant region (CH1), and a light chain constant region (CL), wherein the C-terminus of the VH region is connected to the N-terminus of the CH1 region, and the C-terminus of the VL region is connected to the N-terminus of the CL region.

In some embodiments, the polypeptide molecule of the present disclosure comprises at least one binding domain. In some embodiments, the binding domain is a CrossFab comprising a VH region, a VL region, a first CH1 region, and a CL region, wherein the C-terminus of the VH region is connected to the N-terminus of the CL region, and the C-terminus of the VL region is connected to the N-terminus of the CH1 region. In some embodiments, the CH1 region and the CL region are connected by a disulfide bond. In some embodiments, the binding domain is a charge steering Fab or charge steering CrossFab. In some embodiments, a CH1 region of the charge steering Fab or charge steering CrossFab comprises mutations K147E and/or K213E according to EU numbering, and a CL region of the charge steering Fab or charge steering CrossFab comprises mutations E123R and/or Q124K according to EU numbering.

In some embodiments, the polypeptide molecule comprises at least one binding domain, and the binding domain comprises: (a) an immunoglobulin heavy chain variable (VH) region comprising a heavy chain complementarity determining region (CDR) 1 (HCDR1), a heavy chain CDR2 (HCDR2), and a heavy chain CDR3 (HCDR3); and (b) an immunoglobulin light chain variable (VL) region comprising a light chain complementarity determining region (CDR) 1 (LCDR1), a light chain CDR2 (LCDR2), and a light chain CDR3 (LCDR3). In some embodiments, the polypeptide molecule comprises at least one binding domain, and the binding domain comprises: (a) a VH region comprising a HCDR1, a HCDR2, and a HCDR3; and (b) a VL region comprising a LCDR1, a LCDR2, and a LCDR3.

In some embodiments, the polypeptide molecule comprises at least one binding domain, and the binding domain comprises a light chain. In some embodiments, the light chain is a kappa light chain. In some embodiments, the light chain is a lambda light chain.

The polypeptide molecules described herein can be subject to modification to generate enhanced polypeptide molecules. The polypeptide molecules may be modified via a number of methodologies. Amino acid sequence modification(s) of sequences coding for the disclosed polypeptide molecules, and their binding domains and/or half-life extension moieties, are contemplated. These modifications can increase bio-longevity, alter any secretory characteristics, etc. In addition, it may be desirable to improve the binding affinity of binding domains to their corresponding binding targets; it may also be desirable to improve other biological properties, including but not limited to specificity, thermostability, expression level, or solubility.

In some embodiments, the polypeptide molecules, binding domains, or half-life extension moieties provided herein are chemically modified, for example, by the covalent attachment of any type of molecule to the polypeptide molecules, binding domains, or half-life extension moieties. Exemplary non-limiting modifications include glycosylation, acetylation, pegylation, phosphorylation, amidation, derivatization by known protecting/blocking groups, proteolytic cleavage, linkage to a cellular ligand or other protein, etc.

In some embodiments, the modification in the polypeptide molecule, or their binding domain or half-life extension moiety, is a substitution, deletion, or insertion of one or more amino acids that results in a change in the amino acid sequence as compared to a parental or wildtype sequence. The variation may be determined by systematically making insertions, deletions, or substitutions of amino acids in the parental or wildtype sequence and testing the resulting variants for activity exhibited by the parental or wildtype sequence.

Substitution of one or more CDR residues or omission of one or more CDRs is also contemplated. Antibodies have been described in the scientific literature in which one or two CDRs can be dispensed with for binding. Padlan et al. (1995 FASEB J. 9:133-139) analyzed the contact regions between antibodies and their antigens, based on published crystal structures, and concluded that only about one fifth to one third of CDR residues actually contact the antigen. Padlan also found many antibodies in which one or two CDRs had no amino acids in contact with an antigen (see also, Vajdos et al. 2002 J Mol Biol 320:415-428).

CDR residues not contacting antigen can be identified based on previous studies (for example residues H60-H65 in CDRH2 are often not required), from regions of Kabat CDRs lying outside Chothia CDRs, by molecular modeling and/or empirically. If a CDR or residue(s) thereof is omitted, it is usually substituted with an amino acid occupying the corresponding position in another human antibody sequence or a consensus of such sequences. Positions for substitution within CDRs and amino acids to substitute can also be selected empirically.

Amino acid substitutions can be the result of replacing one amino acid with another amino acid having similar structural and/or chemical properties, such as the substitution of a leucine with a serine, e.g., conservative amino acid replacements. Standard techniques known to those of skill in the art can be used to introduce mutations in the nucleotide sequence encoding a polypeptide molecule provided herein, including, for example, site-directed mutagenesis and PCR-mediated mutagenesis which results in amino acid substitutions.

A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g, tyrosine, phenylalanine, tryptophan, histidine). Table 1 below lists exemplary conservative amino acid substitutions.

TABLE 1
Conservative Substitutions

| Original AA | Conservative Substitution |
|---|---|
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Alternatively, mutations in the polypeptide molecule, binding domain, or half-life extension moiety can be introduced randomly along all or part of the coding sequence, such as by saturation mutagenesis, and the resultant mutants can be screened for biological activity to identify mutants that retain activity. Following mutagenesis, the encoded protein can be expressed, and the activity of the protein can be determined.

Substantial modifications in the biological properties of the polypeptide molecule are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. The substitutions which in general are expected to produce the greatest changes in the polypeptide properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

In some embodiments, the polypeptide molecules, binding domains and/or half-life extension moieties comprise one or more non-classical amino acids.

Provided are also polypeptide molecules, binding domains and/or half-life extension moieties comprising one or more a small amino acids. Exemplary small amino acids comprise alanine, glycine, serine, aspartic acid, cysteine, proline, and threonine. A small amino acid can comprise a volume up to about 117 A³.

In some embodiments, the polypeptide molecules, binding domains and/or half-life extension moieties comprise one or more flexible amino acids. A flexible amino acid can comprise beta-turn forming ability. Exemplary flexible amino acids comprise glycine, serine, asparagine, and aspartic acid, are β-turn-forming amino acids. A flexible amino acid can also comprise an amino acid that lacks a side chain or comprises a reduced side chain. Thus, alanine is a flexible amino acid as it comprises a reduced side chain that allows for flexibility. A flexible amino acid can also comprise a structure that allows for kinking. Exemplary amino acids that can kick comprise proline.

In some embodiments, the polypeptide molecule comprises a small and flexible amino acid. In some embodiments, the small and flexible amino acid comprises glycine. In some embodiments, the small and flexible amino acid comprises alanine.

Substitutions in the polypeptide molecules described herein may be in the range of about 1 to 100 amino acids, including all ranges and subranges therebetween. In some embodiments, the substitution includes fewer than about 100 amino acid substitutions, fewer than about 95 amino acid substitutions, fewer than about 90 amino acid substitutions, fewer than about 85 amino acid substitutions, fewer than about 80 amino acid substitutions, fewer than about 75 amino acid substitutions, fewer than about 70 amino acid substitutions, fewer than about 65 amino acid substitutions, fewer than about 50 amino acid substitutions, fewer than about 45 amino acid substitutions, fewer than about 40 amino acid substitutions, fewer than about 35 amino acid substitutions, fewer than about 30 amino acid substitutions, fewer than about 25 amino acid substitutions, fewer than about 20 amino acid substitutions, fewer than about 15 amino acid substitutions, fewer than about 10 amino acid substitutions, fewer than about 5 amino acid substitutions, fewer than about 4 amino acid substitutions, fewer than about 3 amino acid substitutions, or fewer than about 2 amino acid substitutions relative to the original molecule.

Amino acid sequence insertions include amino- and/or carboxyl-terminal fusions ranging in length from 1 residue to polypeptides containing 100 or more residues, as well as intrasequence insertions of single or multiple amino acid residues. Examples of terminal insertions include an antibody with an N-terminal methionyl residue. Other insertional variants of the antibody molecule include the fusion to the N or C terminus of the antibody to an enzyme (e.g., for antibody-directed enzyme prodrug therapy) or a polypeptide which increases the serum half-life of the antibody. In some embodiments, the insertion is about 1 amino acid, about 2 amino acids, about 3 amino acids, about 4 amino acids, about 5 amino acids, about 6 amino acids, about 7 amino acids, about 8 amino acids, about 9 amino acids, about 10 amino acids, about 11 amino acids, about 12 amino acids, about 13 amino acids, about 14 amino acids, about 15 amino acids, about 16 amino acids, about 17 amino acids, about 18 amino acids, about 19 amino acids, about 20 amino acids, about 21 amino acids, about 22 amino acids, about 23 amino acids, about 24 amino acids, about 25 amino acids, about 26 amino acids, about 27 amino acids, about 28 amino acids, about 29 amino acids, about 30 amino acids, about 31 amino acids, about 32 amino acids, about 33 amino acids, about 34 amino acids, about 35 amino acids, about 36 amino acids, about 37 amino acids, about 38 amino acids, about 39 amino acids, about 40 amino acids, about 41 amino acids, about 42 amino acids, about 43 amino acids, about 44 amino acids, about 45 amino acids, about 46 amino acids, about 47 amino acids, about 48 amino acids, about 49 amino acids, about 50 amino acids, about 51 amino acids, about 52 amino acids, about 53 amino acids, about 54 amino acids, about 55 amino acids, about 56 amino acids, about 57 amino acids, about 58 amino acids, about 59 amino acids, about 60 amino acids, about 61 amino acids, about 62 amino acids, about 63 amino acids, about 64 amino acids, about 65 amino acids, about 66 amino acids, about 67 amino acids, about 68 amino acids, about 69 amino acids, about 70 amino acids, about 71 amino acids, about 72 amino acids, about 73 amino acids, about 74 amino acids, about 75 amino acids, about 76 amino acids, about 77 amino acids, about 78 amino acids, about 79 amino acids, about 80 amino acids, about 81 amino acids, about 82 amino acids, about 83 amino acids, about 84 amino acids, about 85 amino acids, about 86 amino acids, about 87 amino acids, about 88 amino acids, about 89 amino acids, about 90 amino acids, about 91 amino acids, about 92 amino acids, about 93 amino acids, about 94 amino acids, about 95 amino acids, about 96 amino acids, about 97 amino acids, about 98 amino acids, about 99 amino acids, or about 100 amino acids, including all ranges and subranges therebetween.

Amino acid sequence deletions include amino- and/or carboxyl-terminal deletions ranging in length from one residue to polypeptides containing a hundred or more residues, as well as intrasequence deletions of single or multiple amino acid residues. In some embodiments, the deletion is about 1 amino acid to about 100 amino acids. In some embodiments, the deletion is about 1 amino acid, about 2 amino acids, about 3 amino acids, about 4 amino acids, about 5 amino acids, about 6 amino acids, about 7 amino acids, about 8 amino acids, about 9 amino acids, about 10 amino acids, about 11 amino acids, about 12 amino acids, about 13 amino acids, about 14 amino acids, about 15 amino acids, about 16 amino acids, about 17 amino acids, about 18 amino acids, about 19 amino acids, about 20 amino acids, about 21 amino acids, about 22 amino acids, about 23 amino acids, about 24 amino acids, about 25 amino acids, about 26 amino acids, about 27 amino acids, about 28 amino acids, about 29 amino acids, about 30 amino acids, about 31 amino acids, about 32 amino acids, about 33 amino acids, about 34 amino acids, about 35 amino acids, about 36 amino acids, about 37 amino acids, about 38 amino acids, about 39 amino acids, about 40 amino acids, about 41 amino acids, about 42 amino acids, about 43 amino acids, about 44 amino acids, about 45 amino acids, about 46 amino acids, about 47 amino acids, about 48 amino acids, about 49 amino acids, about 50 amino acids, about 51 amino acids, about 52 amino acids, about 53 amino acids, about 54 amino acids, about 55 amino acids, about 56 amino acids, about 57 amino acids, about 58 amino acids, about 59 amino acids, about 60 amino acids, about 61 amino acids, about 62 amino acids, about 63 amino acids, about 64 amino acids, about 65 amino acids, about 66 amino acids, about 67 amino acids, about 68 amino acids, about 69 amino acids, about 70 amino acids, about 71 amino acids, about 72 amino acids, about 73 amino acids, about 74 amino acids, about 75 amino acids, about 76 amino acids, about 77 amino acids, about 78 amino acids, about 79 amino acids, about 80 amino acids, about 81 amino acids, about 82 amino acids, about 83 amino acids, about 84 amino acids, about 85 amino acids, about 86 amino acids, about 87 amino acids, about 88 amino acids, about 89 amino acids, about 90 amino acids, about 91 amino acids, about 92 amino acids, about 93 amino acids, about 94 amino acids, about 95 amino acids, about 96 amino acids, about 97 amino acids, about 98 amino acids, about 99 amino acids, or about 100 amino acids, including all ranges and subranges therebetween.

In some embodiments, the polypeptide molecule comprises a TCRβV-binding domain. In some embodiments, the polypeptide molecule comprising the TCRβV-binding domain is an scFv. In some embodiments, the polypeptide molecule comprising the TCRβV-binding domain comprises a Fab.

In some embodiments, the polypeptide molecule comprises a TCRβV6-5-binding domain. In some embodiments, the polypeptide molecule comprising the TCRβV6-5-binding domain is an scFv. In some embodiments, the polypeptide molecule comprising the TCRβV6-5-binding domain comprises a Fab.

In some embodiments, the polypeptide molecule comprises a TCRβV12-3-binding domain. In some embodiments, the polypeptide molecule comprising the TCRβV12-3-binding domain is an scFv. In some embodiments, the polypeptide molecule comprising the TCRβV12-3-binding domain comprises a Fab.

In some embodiments, the polypeptide molecule comprises a TCRβC-binding domain. In some embodiments, the polypeptide molecule comprising the TCRβC-binding domain is an scFv. In some embodiments, the polypeptide molecule comprising the TCRβC-binding domain comprises a Fab.

In some embodiments, the polypeptide molecule comprises a TCRβC1-binding domain. In some embodiments, the polypeptide molecule comprising the TCRβVC1-binding domain is an scFv. In some embodiments, the polypeptide molecule comprising the TCRβC1-binding domain comprises a Fab.

In some embodiments, the polypeptide molecule comprises a TCRβC2-binding domain. In some embodiments, the polypeptide molecule comprising the TCRβC2-binding domain is an scFv. In some embodiments, the polypeptide molecule comprising the TCRβC2-binding domain comprises a Fab.

In some embodiments, the present disclosure provides a polypeptide molecule comprising a first binding domain and a second binding domain. In some embodiments, the first binding domain is a TCRβ-binding domain (e.g., a TCRβV or a TCRβC). In some embodiments, the second binding domain is a TAA-binding domain (e.g., a PSMA-binding domain or a CD19-binding domain). In some embodiments, the TCRβ-binding domain is an scFv. In some embodiments, the TAA-binding domain comprises a Fab comprising a Fab heavy chain and a Fab light chain.

In some embodiments, the present disclosure provides a polypeptide molecule comprising a first binding domain and a second binding domain. In some embodiments, the first binding domain is a TCRβC-binding domain (e.g., a TCRβC1-binding domain or a TCRβC2-binding domain). In some embodiments, the second binding domain is a TAA-binding domain (e.g., a PSMA-binding domain or a CD19-binding domain).

In some embodiments, the polypeptide molecule comprises: (i) a TCRβ-binding domain; (ii) a binding domain linker; and (iii) a TAA-binding domain. In some embodiments, the polypeptide molecule comprises: (i) a TCRβV-binding domain; (ii) a binding domain linker; and (iii) a TAA-binding domain. In some embodiments, the polypeptide molecule comprises: (i) a TCRβC-binding domain; (ii) a binding domain linker; and (iii) a TAA-binding domain. In some embodiments, the TCRβ-binding domain (e.g., a TCRβV-binding domain or a TCRβC-binding domain) is an scFv. In some embodiments, the TAA-binding domain comprises a Fab. In some embodiments, the binding domain linker connects the C-terminus of the TCRβV or TCRβV-binding domain to the N-terminus of the TAA-binding domain. In some embodiments, the binding domain linker connects the N-terminus of the TCRβV or TCRβV-binding domain to the C-terminus of the TAA-binding domain.

In some embodiments, the polypeptide molecule comprises: (i) a TCRβV-binding domain; (ii) a binding domain linker; and (iii) a TAA-binding domain. In some embodiments, the polypeptide molecule comprises in order from N-terminus to C-terminus: (i) a TCRβV-binding domain; (ii) a binding domain linker; and (iii) a TAA-binding domain. In some embodiments, the polypeptide molecule comprises in order from N-terminus to C-terminus: (i) a TAA-binding domain; (ii) a binding domain linker; and (iii) a TCRβV-binding domain. In some embodiments, the TCRβV-binding domain is an scFv. In some embodiments, the TAA-binding domain comprises a Fab.

In some embodiments, the polypeptide molecule comprises: (i) a TCRβV6-5-binding domain; (ii) a binding domain linker; and (iii) a TAA-binding domain. In some embodiments, the polypeptide molecule comprises in order from N-terminus to C-terminus: (i) a TCRβV6-5-binding domain; (ii) a binding domain linker; and (iii) a TAA-binding domain. In some embodiments, the polypeptide molecule comprises in order from N-terminus to C-terminus: (i) a TAA-binding domain; (ii) a binding domain linker; and (iii) a TCRβV6-5-binding domain. In some embodiments, the TCRβV6-5-binding domain is an scFv. In some embodiments, the TAA-binding domain comprises a Fab.

In some embodiments, the polypeptide molecule comprises: (i) a TCRβV12-3-binding domain; (ii) a binding domain linker; and (iii) a TAA-binding domain. In some embodiments, the polypeptide molecule comprises in order from N-terminus to C-terminus: (i) a TCRβV12-3-binding domain; (ii) a binding domain linker; and (iii) a TAA-binding domain. In some embodiments, the polypeptide molecule comprises in order from N-terminus to C-terminus: (i) a TAA-binding domain; (ii) a binding domain linker; and (iii) a TCRβV12-3-binding domain. In some embodiments, the TCRβV12-3-binding domain is an scFv. In some embodiments, the TAA-binding domain comprises a Fab.

In some embodiments, the polypeptide molecule comprises: (i) a TCRβ-binding domain; (ii) a binding domain linker; and (iii) a B cell-binding domain. In some embodiments, the polypeptide molecule comprises: (i) a TCRβV-binding domain; (ii) a binding domain linker; and (iii) a B cell-binding domain. In some embodiments, the polypeptide molecule comprises: (i) a TCRβC-binding domain; (ii) a binding domain linker; and (iii) a B cell-binding domain. In some embodiments, the TCRβ-binding domain is an scFv. In some embodiments, the B cell-binding domain comprises a Fab. In some embodiments, the binding domain linker connects the C-terminus of the TCRβ-binding domain to the N-terminus of the B cell-binding domain. In some embodiments, the binding domain linker connects the N-terminus of the TCRβ-binding domain to the C-terminus of the B cell-binding domain.

In some embodiments, the polypeptide molecule comprises: (i) a TCRβV-binding domain; (ii) a binding domain linker; and (iii) a B cell-binding domain. In some embodiments, the polypeptide molecule comprises in order from N-terminus to C-terminus: (i) a TCRβV-binding domain; (ii) a binding domain linker; and (iii) a B cell-binding domain. In some embodiments, the polypeptide molecule comprises in order from N-terminus to C-terminus: (i) a B cell-binding domain; (ii) a binding domain linker; and (iii) a TCRβV-binding domain. In some embodiments, the TCRβV-binding domain is an scFv. In some embodiments, the B cell-binding domain comprises a Fab.

In some embodiments, the polypeptide molecule comprises: (i) a TCRβV-binding domain; (ii) a binding domain linker; and (iii) a CD19-binding domain. In some embodiments, the polypeptide molecule comprises in order from N-terminus to C-terminus: (i) a TCRβV-binding domain; (ii) a binding domain linker; and (iii) a CD19-binding domain. In some embodiments, the polypeptide molecule comprises in order from N-terminus to C-terminus: (i) a CD19-binding domain; (ii) a binding domain linker; and (iii) a TCRβV-binding domain. In some embodiments, the TCRβV-binding domain is an scFv. In some embodiments, the CD19-binding domain comprises a Fab.

In some embodiments, the polypeptide molecule comprises: (i) a TCRβV6-5-binding domain; (ii) a binding domain linker; and (iii) a CD19-binding domain. In some embodiments, the polypeptide molecule comprises in order from N-terminus to C-terminus: (i) a TCRβV6-5-binding domain; (ii) a binding domain linker; and (iii) a CD19-binding domain. In some embodiments, the polypeptide molecule comprises in order from N-terminus to C-terminus: (i) a CD19-binding domain; (ii) a binding domain linker; and (iii) a TCRβV6-5-binding domain. In some embodiments, the TCRβV6-5-binding domain is an scFv. In some embodiments, the CD19-binding domain comprises a Fab.

In some embodiments, the polypeptide molecule comprises: (i) a TCRβV12-3-binding domain; (ii) a binding domain linker; and (iii) a CD19-binding domain. In some embodiments, the polypeptide molecule comprises in order from N-terminus to C-terminus: (i) a TCRβV12-3-binding domain; (ii) a binding domain linker; and (iii) a CD19-binding domain. In some embodiments, the polypeptide molecule comprises in order from N-terminus to C-terminus: (i) a CD19-binding domain; (ii) a binding domain linker; and (iii) a TCRβV12-3-binding domain. In some embodiments, the TCRβV12-3-binding domain is an scFv. In some embodiments, the CD19-binding domain comprises a Fab.

In some embodiments, the polypeptide molecule comprises: (i) a TCRβC-binding domain; (ii) a binding domain linker; and (iii) a TAA-binding domain. In some embodiments, the polypeptide molecule comprises in order from N-terminus to C-terminus: (i) a TCRβC-binding domain; (ii) a binding domain linker; and (iii) a TAA-binding domain. In some embodiments, the polypeptide molecule comprises in order from N-terminus to C-terminus: (i) a TAA-binding domain; (ii) a binding domain linker; and (iii) a TCRβC-binding domain. In some embodiments, the TCRβC-binding domain is an scFv. In some embodiments, the TAA-binding domain comprises a Fab.

In some embodiments, the polypeptide molecule comprises: (i) a TCRβV5-5/6 binding domain; (ii) a binding domain linker; and (iii) a PSMA-binding domain. In some embodiments, the polypeptide molecule comprises: (i) a TCRβV6-5-binding domain; (ii) a binding domain linker; and (iii) a PSMA-binding domain. In some embodiments, the polypeptide molecule comprises: (i) a TCRβV12-3/4 binding domain; (ii) a binding domain linker; and (iii) a PSMA-binding domain. In some embodiments, the polypeptide molecule comprises: (i) a TCRβV20-1 binding domain; (ii) a binding domain linker; and (iii) a PSMA-binding domain. In some embodiments, the polypeptide molecule comprises: (i) a TCRβC1-binding domain; (ii) a binding domain linker; and (iii) a PSMA-binding domain. In some embodiments, the polypeptide molecule comprises: (i) a TCRβC2-binding domain; (ii) a binding domain linker; and (iii) a PSMA-binding domain. In some embodiments, the binding domain linker connects the C-terminus of the TCRβ-binding domain to the N-terminus of the PSMA-binding domain. In some embodiments, the binding domain linker connects the N-terminus of the TCRβ-binding domain to the C-terminus of the PSMA-binding domain.

In some embodiments, the polypeptide molecule comprises: (i) an anti-TCRβV5-5/6 scFv; (ii) a binding domain linker; and (iii) an anti-PSMA Fab. In some embodiments, the polypeptide molecule comprises: (i) an anti-TCRβV6-5 scFv; (ii) a binding domain linker; and (iii) an anti-PSMA Fab. In some embodiments, the polypeptide molecule comprises: (i) an anti-TCRβV12-3/4 scFv; (ii) a binding domain linker; and (iii) an anti-PSMA Fab. In some embodiments, the polypeptide molecule comprises: (i) an anti-TCRβV20-1 scFv; (ii) a binding domain linker; and (iii) an anti-PSMA Fab. In some embodiments, the polypeptide molecule comprises: (i) an anti-TCRβC1 scFv; (ii) a binding domain linker; and (iii) an anti-PSMA Fab. In some embodiments, the polypeptide molecule comprises: (i) an anti-TCRβC2 scFv; (ii) a binding domain linker; and (iii) an anti-PSMA Fab. In some embodiments, the binding domain linker connects the C-terminus of the anti-TCRβ scFv VH region to the N-terminus of the anti-PSMA Fab heavy chain. In some embodiments, the binding domain linker connects the C-terminus of the anti-TCRβ scFv VL region to the N-terminus of the anti-PSMA Fab heavy chain. In some embodiments, the binding domain linker connects the C-terminus of the anti-TCRβ scFv VH region to the N-terminus of the anti-PSMA Fab light chain. In some embodiments, the binding domain linker connects the C-terminus of the anti-TCRβ scFv VL region to the N-terminus of the anti-PSMA Fab light chain. In some embodiments, the binding domain linker connects the N-terminus of the anti-TCRβ scFv VH region to the C-terminus of the anti-PSMA Fab heavy chain. In some embodiments, the binding domain linker connects the N-terminus of the anti-TCRβ scFv VL region to the C-terminus of the anti-PSMA Fab heavy chain. In some embodiments, the binding domain linker connects the N-terminus of the anti-TCRβ scFv VH region to the C-terminus of the anti-PSMA Fab light chain. In some embodiments, the binding domain linker connects the N-terminus of the anti-TCRβ scFv VL region to the C-terminus of the anti-PSMA Fab light chain.

In some embodiments, the polypeptide molecule comprises: (i) a TCRβV5-5/6 binding domain; (ii) a binding domain linker; and (iii) a CD19-binding domain. In some embodiments, the polypeptide molecule comprises: (i) a TCRβV6-5-binding domain; (ii) a binding domain linker; and (iii) a CD19-binding domain. In some embodiments, the polypeptide molecule comprises: (i) a TCRβV12-3/4 binding domain; (ii) a binding domain linker; and (iii) a CD19-binding domain. In some embodiments, the polypeptide molecule comprises: (i) a TCRβV20-1 binding domain; (ii) a binding domain linker; and (iii) a CD19-binding domain. In some embodiments, the polypeptide molecule comprises: (i) a TCRβC1-binding domain; (ii) a binding domain linker; and (iii) a CD19-binding domain. In some embodiments, the polypeptide molecule comprises: (i) a TCRβC2-binding domain; (ii) a binding domain linker; and (iii) a CD19-binding domain. In some embodiments, the binding domain linker connects the C-terminus of the TCRβ-binding domain to the N-terminus of the CD19-binding domain. In some embodiments, the binding domain linker connects the N-terminus of the TCRβ-binding domain to the C-terminus of the CD19-binding domain.

In some embodiments, the polypeptide molecule comprises: (i) an anti-TCRβV5-5/6 scFv; (ii) a binding domain linker; and (iii) an anti-CD19 Fab. In some embodiments, the polypeptide molecule comprises: (i) an anti-TCRβV6-5 scFv; (ii) a binding domain linker; and (iii) an anti-CD19 Fab. In some embodiments, the polypeptide molecule comprises: (i) an anti-TCRβV12-3/4 scFv; (ii) a binding domain linker; and (iii) an anti-CD19 Fab. In some embodiments, the polypeptide molecule comprises: (i) an anti-TCRβV20-1 scFv; (ii) a binding domain linker; and (iii) an anti-CD19 Fab. In some embodiments, the polypeptide molecule comprises: (i) an anti-TCRβC1 scFv; (ii) a binding domain linker; and (iii) an anti-CD19 Fab. In some embodiments, the polypeptide molecule comprises: (i) an anti-TCRβC2 scFv; (ii) a binding domain linker; and (iii) an anti-CD19 Fab. In some embodiments, the binding domain linker connects the C-terminus of the anti-TCRβ scFv VH region to the N-terminus of the anti-CD19 Fab heavy chain. In some embodiments, the binding domain linker connects the C-terminus of the anti-TCRβ scFv VL region to the N-terminus of the anti-CD19 Fab heavy chain. In some embodiments, the binding domain linker connects the C-terminus of the anti-TCRβ scFv VH region to the N-terminus of the anti-CD19 Fab light chain. In some embodiments, the binding domain linker connects the C-terminus of the anti-TCRβ scFv VL region to the N-terminus of the anti-CD19 Fab light chain. In some embodiments, the binding domain linker connects the N-terminus of the anti-TCRβ scFv VH region to the C-terminus of the anti-CD19 Fab heavy chain. In some embodiments, the binding domain linker connects the N-terminus of the anti-TCRβ scFv VL region to the C-terminus of the anti-CD19 Fab heavy chain. In some embodiments, the binding domain linker connects the N-terminus of the anti-TCRβ scFv VH region to the C-terminus of the anti-CD19 Fab light chain. In some embodiments, the binding domain linker connects the N-terminus of the anti-TCRβ scFv VL region to the C-terminus of the anti-CD19 Fab light chain.

In some embodiments, the polypeptide molecule is a bispecific polypeptide molecule comprising a TCRβV6-5-binding domain and a CD19-binding domain. In some embodiments, the bispecific polypeptide molecule comprises a heavy chain comprising an amino acid sequence with at least about 70%, at least about 80%, at least about 85%, at least about 90%, at least about 95%, at least about 96%, at least about 97%, at least about 98%, at least about 99%, or 100% identity to SEQ ID NO: 1412. In some embodiments, the bispecific polypeptide molecule comprises a heavy chain comprising an amino acid sequence with at least about 70%, at least about 80%, at least about 85%, at least about 90%, at least about 95%, at least about 96%, at least about 97%, at least about 98%, at least about 99%, or 100% identity to any one of SEQ ID NOs: 1417, 1418, and 1421-1423. In some embodiments, the bispecific polypeptide molecule comprises a light chain comprising an amino acid sequence with at least about 70%, at least about 80%, at least about 85%, at least about 90%, at least about 95%, at least about 96%, at least about 97%, at least about 98%, at least about 99%, or 100% identity to any one of SEQ ID NOs: 1369, 1416, 1419, and 1420. In some embodiments, the bispecific polypeptide molecule comprises (i) a first heavy chain comprising an amino acid sequence with at least about 70%, at least about 80%, at least about 85%, at least about 90%, at least about 95%, at least about 96%, at least about 97%, at least about 98%, at least about 99%, or 100% identity to SEQ ID NO: 1412; (ii) a second heavy chain comprising an amino acid sequence with at least about 70%, at least about 80%, at least about 85%, at least about 90%, at least about 95%, at least about 96%, at least about 97%, at least about 98%, at least about 99%, or 100% identity to SEQ ID NO: 1417, 1418, and 1421-1423; and (ii) a light chain comprising an amino acid sequence with at least about 70%, at least about 80%, at least about 85%, at least about 90%, at least about 95%, at least about 96%, at least about 97%, at least about 98%, at least about 99%, or 100% identity to SEQ ID NO: 1369, 1416, 1419, and 1420.

In some embodiments, the polypeptide molecule comprises: a first heavy chain (HC1) comprising an amino acid sequence of SEQ ID NO: 1412; a light chain that comprises an amino acid sequence of SEQ ID NO: 1416, and a second heavy chain (HC2) that comprises an amino acid sequence of SEQ ID NO: 1417. In some embodiments, the polypeptide molecule comprises: a first heavy chain (HC1) comprising an amino acid sequence of SEQ ID NO: 1412; a light chain that comprises an amino acid sequence of SEQ ID NO: 1369, and a second heavy chain (HC2) that comprises an amino acid sequence of SEQ ID NO: 1418. In some embodiments, the polypeptide molecule comprises: a first heavy chain (HC1) comprising an amino acid sequence of SEQ ID NO: 1412; a light chain that comprises an amino acid sequence of SEQ ID NO: 1419, and a second heavy chain (HC2) that comprises an amino acid sequence of SEQ ID NO: 1417. In some embodiments, the polypeptide molecule comprises: a first heavy chain (HC1) comprising an amino acid sequence of SEQ ID NO: 1412; a light chain that comprises an amino acid sequence of SEQ ID NO: 1420, and a second heavy chain (HC2) that comprises an amino acid sequence of SEQ ID NO: 1417. In some embodiments, the polypeptide molecule comprises: a first heavy chain (HC1) comprising an amino acid sequence of SEQ ID NO: 1412; a light chain that comprises an amino acid sequence of SEQ ID NO: 1369, and a second heavy chain (HC2) that comprises an amino acid sequence of SEQ ID NO: 1421. In some embodiments, the polypeptide molecule comprises: a first heavy chain (HC1) comprising an amino acid sequence of SEQ ID NO: 1412; a light chain that comprises an amino acid sequence of SEQ ID NO: 1369, and a second heavy chain (HC2) that comprises an amino acid sequence of SEQ ID NO: 1422. In some embodiments, the polypeptide molecule comprises: a first heavy chain (HC1) comprising an amino acid sequence of SEQ ID NO: 1412; a light chain that comprises an amino acid sequence of SEQ ID NO: 1369, and a second heavy chain (HC2) that comprises an amino acid sequence of SEQ ID NO: 1423.

### TCRβV-Binding Domains

In some embodiments, the polypeptide molecule of the disclosure specifically binds a T cell receptor (TCR). TCRs recognize foreign antigens, which have been processed as small peptides and bound to major histocompatibility complex (MHC) molecules at the surface of antigen presenting cells (APC). Each TCR is a dimer of one alpha and one beta chain or one delta and one gamma chain. In a single cell, the TCR loci are rearranged and expressed in the order delta, gamma, beta, and alpha. If both delta and gamma rearrangements produce functional chains, the cell expresses delta and gamma. If not, the cell proceeds to rearrange the beta and alpha loci. This region represents the germline organization of the TCR beta locus. The beta locus includes V (variable), J (joining), diversity (D), and C (constant) segments. Recombination of many different V segments with several J segments provides a wide range of antigen recognition. The beta locus also includes eight trypsinogen genes, three of which encode functional proteins and five of which are pseudogenes. Chromosomal abnormalities involving the TCR beta locus have been associated with T cell lymphomas.

Provided herein are polypeptide molecules comprising TCRβV or TCRβC-binding domains. In some embodiments, the polypeptide molecule specifically binds TCRβV. In some embodiments, the polypeptide molecule specifically binds TCRβC. In some embodiments, the polypeptide molecule is a bispecific polypeptide molecule and the bispecific polypeptide molecule specifically binds a TAA (e.g., CD19 or PSMA) and TCRβV (e.g., TCRβV6-5 and TCRβV12-3). In some embodiments, the TCRβV or TCRβC-binding domain comprises a Fab comprising a Fab heavy chain and Fab light chain. In some embodiments, the TCRβV or TCRβC-binding domain is an scFv.

In some embodiments, the polypeptide molecule of the disclosure binds TCRβV. In some embodiments, the polypeptide molecule comprises a TCRβV-binding domain. In some embodiments, the TCRβV-binding domain is an scFv. In some embodiments, the TCRβV-binding domain comprises a Fab.

In some embodiments, the TCRβV-binding domain selectively binds to human TCRβV (e.g., a TCRβV family, subfamily, or variant thereof). TCRβV families and subfamilies are known in the art, e.g., as described in Yassai et al., (2009) Immunogenetics 61(7) pp:493-502; and Wei S. and Concannon P. (1994) Human Immunology 41(3) pp: 201-206. Different gene nomenclatures for any one of the TCRβ families and subfamilies described herein are also encompassed by the present disclosure. For example, the TCRβV nomenclature as described by Folch, G. and Lefranc, M.-P., Exp. Clin. Immunogenet, 17, 42-54 (2000); Lefranc, M.-P. and Lefranc, G., The T Cell Receptor FactsBook, Academic Press, Harcourt, 398 pages (2001); Slightom, J. L. et al., Genomics, 20, 149-168 (1994); Wei et al., Immunogenetics, 40, 27-36 (1994); Arden, B. et al., Immunogenetics, 42, 455-500 (1995); Rowen, L. et al., Science, 272, 1755-1762 (1996); and Wilson, R. K. et al., Immunological Reviews 101, 149-172 (1988) are encompassed by the present disclosure, each of which are hereby incorporated by reference in their entirety.

In some embodiments, the TCRβV-binding domain selectively binds to any one of: TCRβV1, TCRβV2, TCRβV3, TCRβV4, TCRβV5, TCRβV6, TCRβV7, TCRβV8, TCRβV9, TCRβV10, TCRβV11, TCRβV12, TCRβV13, TCRβV14, TCRβV15, TCRβV16, TCRβV17, TCRβV18, TCRβV19, TCRβV20, TCRβV21, TCRβV22, TCRβV23, TCRβV24, TCRβV25, TCRβV26, TCRβV27, TCRβV28, TCRβV29, TCRβV30, TCRβVA, TCRβVB, TCRβVC, or a variant thereof. See, also, Folch and Lefranc, Exp. Clin. Immunogenet, 17, 42-54 (2000).

In some embodiments, the TCRβV-binding domain selectively binds to any one of TCRβV2*01, TCRβV2*02, TCRβV2*03, TCRβV3-1*01, TCRβV3-1*02, TCRβV4-1*01, TCRβV4-1*02, TCRβV4-2*01, TCRβV4-2*02, TCRβV4-3*01, TCRβV4-3*02, TCRβV4-3*03, TCRβV4-3*04, TCRβV5-1*01, TCRβV5-1*02, TCRβV5-4*01, TCRβV5-4*02, TCRβV5-4*03, TCRβV5-4*04, TCRβV5-5*01, TCRβV5-5*02, TCRβV5-5*03, TCRβV5-6*01, TCRβV5-8*01, TCRβV5-8*02, TCRβV6-1*01, TCRβV6-2*01, TCRβV6-3*01, TCRβV6-4*01, TCRβV6-4*02, TCRβV6-5*01, TCRβV6-6*01, TCRβV6-6*02, TCRβV6-6*03, TCRβV6-6*04, TCRβV6-6*05, TCRβV6-8*01, TCRβV6-9*01, TCRβV7-2*01, TCRβV7-2*02, TCRβV7-2*03, TCRβV7-2*04, TCRβV7-3*01, TCRβV7-3*04, TCRβV7-3*05, TCRβV7-4*01, TCRβV7-6*01, TCRβV7-6*02, TCRβV7-7*01, TCRβV7-7*02, TCRβV7-8*01, TCRβV7-8*02, TCRβV7-8*03, TCRβV7-9*01, TCRβV7-9*02, TCRβV7-9*03, TCRβV7-9*04, TCRβV7-9*05, TCRβV7-9*06, TCRβV7-9*07, TCRβV9*01, TCRβV9*02, TCRβV9*03, TCRβV10-1*01, TCRβV10-1*02, TCRβV10-2*01, TCRβV10-2*02, TCRβV10-3*01, TCRβV10-3*02, TCRβV10-3*03, TCRβV10-3*04, TCRβV11-1*01, TCRβV11-2*01, TCRβV11-2*02, TCRβV11-2*03, TCRβV11-3*01, TCRβV11-3*02, TCRβV11-3*03, TCRβV11-3*04, TCRβV12-3*01, TCRβV12-4*01, TCRβV12-4*02, TCRβV12-5*01, TCRβV13*01, TCRβV13*02, TCRβV14*01, TCRβV14*02, TCRβV15*01, TCRβV15*02, TCRβV15*03, TCRβV16*01, TCRβV16*03, TCRβV18*01, TCRβV19*01, TCRβV19*02, TCRβV19*03, TCRβV20-1*01, TCRβV20-1*02, TCRβV20-1*03, TCRβV20-1*04, TCRβV20-1*05, TCRβV20-1*06, TCRβV20-1*07, TCRβV24-1*01, TCRβV25-1*01, TCRβV27*01, TCRβV28*01, TCRβV29-1*01, TCRβV29-1*02, TCRβV29-1*03, TCRβV30*01, TCRβV30*02, TCRβV30*03, TCRβV30*04, or a variant thereof.

Exemplary amino acid sequences for TCRβV family members are shown in Table 2 and can also be found on the ImMunoGeneTics Information System website: www.imgt.org, or in a similar resource.

TABLE 2
Exemplary TCRβV Family Members

| | | | SEQ ID |
|---|---|---|---|
| Target | Species | Amino Acid Sequence | NO: |
| TCRβV5-5 | Human | DAGVTQSPTHLIKTRGQQVTLRCSPISGHKSV | 2230 |
| | | SWYQQVLGQGPQFIFQYYEKEERGRGNFPDR | |
| | | FSARQFPNYSSELNVNALLLGDSALYLCASSL | |
| TCRβV5-5 | Cynomolgus | DGGVTQSPRHLIKTTGQQVTLRCSPISGHTSV | 2231 |
| | Monkey | SWYQQALGQGPQFIFEYEEKERERGNFPDRFS | |
| | | GHQFSNYSSELNVNALEAGDSALYLCASSL | |
| TCRβV5-6 | Human | DAGVTQSPTHLIKTRGQQVTLRCSPKSGHDT | 2232 |
| | | VSWYQQALGQGPQFIFQYYEEEERQRGNFPD | |
| | | RFSGHQFPNYSSELNVNALLLGDSALYLCASS | |
| | | L | |
| TCRβV5-6 | Cynomolgus | DGGVTQSPRHLIKTTGQQVTLRCSPISGHTSV | 2231 |
| | Monkey | SWYQQALGQGPQFIFEYEEKERERGNFPDRFS | |
| | | GHQFSNYSSELNVNALEAGDSALYLCASSL | |
| TCRβV5-6 | Rhesus | DTGVTQSPTHLIRKRGQQVTLRCSPISGHNTV | 2233 |
| | Monkey | SWYQQALGQGPQFIFQYYEKEERERGNFPGR | |
| | | FSGHQFPNYSSELNVSALLLGDSALYLCASSL | |
| TCRβV6-1 | Cynomolgus | NADVTQSPKFQILKTGQSMTLQCAQDMNHN | 2410 |
| (CLY-002) | Monkey | SMYWYRQDPGMGLRLIHYSATEDTTDPGEVP | |
| | | DGYSVSRLNKKEFLLRLESAALSQTSVYFCAS | |
| | | SY | |
| TCRβV6-2 | Cynomolgus | NAGVTQTPKFQVLKTGQNMTLKCAQDMNH | 2411 |
| (CLY-003) | Monkey | DRMYWYRQDPGMGLRLIHYSAGEGSTEKGE | |
| | | VPNGYNASRLNKKDFLLRLESAAPSQTSMYF | |
| | | CASSY | |
| TCRβV6-3 | Cynomolgus | NAGVTQTPKFQVLKTGQSMTLQCAQDMNHD | 2412 |
| (CLY-004) | Monkey | YMYWYRQDPGMGLRLIHYSVGEGSTEKGEV | |
| | | PDGYNVTRSNTEDFPLRLESAAPSQTSVYFCA | |
| | | SSY | |
| TCRβV6-5 | Human | NAGVTQTPKFQVLKTGQSMTLQCAQDMNHE | 223 |
| (CLY-001) | | YMSWYRQDPGMGLRLIHYSVGAGITDQGEV | |
| | | PNGYNVSRSTTEDFPLRLLSAAPSQTSVYFCA | |
| | | SSY | |
| TCRβV6-5 | Cynomolgus | NVGVTQTPKFQVLKTGQSMTVQCAQDMNH | 2234 |
| | Monkey | NFMYWYRQDPGMGLRLIHYSGAAGTTDKGE | |
| | | VPSGYNVSRLNTEDFPLRLESADPSQTSVYIC | |
| | | ASSY | |
| TCRβV6-5 | Rhesus | NVGVTQTPKFQVLKTGQSMTVQCAQDMNH | 2235 |
| | Monkey | NFMYWYRQDPGMGLRLIHYSGAAGTTDKGE | |
| | | VPSGYNVSRLNTEDFPLRLESADP | |
| | | SQTSVYFCASSY | |
| TCRβV6-6 | Cynomolgus | NAGVNQTPKFQVLKTGQSMTLQCAQDMNHE | 2413 |
| (CLY-005) | Monkey | SMSWYRQDPGMGLRLIHYSVGAGITDKGEVP | |
| | | NGYNVSRSNTEDFPLRLESAASSQTSVYFCAS | |
| | | SY | |
| TCRβV12-3 | Human | DAGVIQSPRHEVTEMGQEVTLRCKPISGHNSL | 2236 |
| | | FWYRQTMMRGLELLIYFNNNVPIDDSGMPED | |
| | | RFSAKMPNASFSTLKIQPSEPRDSAVYFCASSL | |
| TCRβV12-3 | Cynomolgus | DAGVIQSPRHEVTEMGKEVTLRCEPISGHTYL | 2237 |
| | Monkey | FWYRQTMMRGLEFLIYFNNKSPIDDSGMPKD | |
| | | RFSAKMPDASFSTLKIQPSEPRDSAVYFCASSL | |
| TCRβV12-3 | Rhesus | DAGVIQSPQHEVTEMGKEVTLRCEPISGHTYL | 2238 |
| | Monkey | FWYRQTMMRGLEFLIYFNNKSPIDDSGMPKD | |
| | | RFSATMPEVSFSTLKIQPSEPRDSAVYFCASSL | |
| TCRβV12- | Human | DAGVIQSPRHEVTEMGQEVTLRCKPISGHNSL | 2414 |
| 3*01 | | FWYRQTMMRGLELLIYFNNNVPIDDSGMPED | |
| | | RFSAKMPNASFSTLKIQPSEPRDSAVYFCASS | |
| | | Y | |
| TCRβV12- | Cynomolgus | DAGVIQSPRHEVTEMGKEVTLRCEPISGHTYL | 2415 |
| 3*01 | Monkey | FWYRQTMMRGLEFLIYFNNKSPIDDSGMPKD | |
| | | RFSAKMPDASFSTLKIQPSEPRDSAVYFCASS | |
| | | Y | |
| TCRβV12-4 | Human | DAGVIQSPRHEVTEMGQEVTLRCKPISGHDY | 2239 |
| | | LFWYRQTMMRGLELLIYFNNNVPIDDSGMPE | |
| | | DRFSAKMPNASFSTLKIQPSEPRDSAVYFCAS | |
| | | SL | |
| TCRβV12-4 | Cynomolgus | DAGVTQTPRHKVTEMGQEVTMRCQPILGHN | 2240 |
| | Monkey | TVFWYRQTVMQGLELLVYLRNKASLDDSGM | |
| | | PKDRFSAEMPDASLATLKIQPSEPRDSAVYLC | |
| | | ASGL | |
| TCRβV12-4 | Rhesus | DAGVTQTPRHKVTEMGQEVTMRCQPILGHN | 2241 |
| | Monkey | TVFWYRQTVMQGLELLVYLRNKASLDDSGM | |
| | | PKDRFSAEMPDASLATLKIQPSEP | |
| | | RDSAVYLCASGL | |
| TCRβV20-1 | Human | GAVVSQHPSWVICKSGTSVKIECRSLDFQATT | 2242 |
| | | MFWYRQFPKQSLMLMATSNEGSKATYEQGV | |
| | | EKDKFLINHASLTLSTLTVTSAHPEDSSFYICS | |
| | | AR | |
| TCRβV20-1 | Cynomolgus | AAVVSQYPSRVICKRGTSVKIECRCLDFQATT | 2243 |
| | Monkey | MFWYRQFQTQSLILMATSNEGSGVTYEQGV | |
| | | KQDKFPINHPNLTFSTLTVTNAHPEDSSFYICS | |
| | | AR | |
| TCRβV20-1 | Rhesus | AAVVSQYPSRVICKRGTSVKIECRCLDFQATT | 2244 |
| | Monkey | MFWYRQFQTQSLILMATSNEGSGVTYEQGV | |
| | | KQDKFPINHPNLTFSTLTVTNAHP | |
| | | EDSSFYICSAR | |

In some embodiments, polypeptide molecule comprises a binding domain that binds human TCRβV5-5. In some embodiments, the binding domain that binds human TCRβV5-5 binds a region of SEQ ID NO: 2230. In some embodiments, the polypeptide molecule comprises a binding domain that binds cynomolgus monkey TCRβV5-5. In some embodiments, the binding domain that binds cynomolgus monkey TCRβV5-5 binds a region of SEQ ID NO: 2231. In some embodiments, a polypeptide molecule binds human TCRβV5-6. In some embodiments, the polypeptide molecule that binds human TCRβV5-6 binds a region of SEQ ID NO: 2232. In some embodiments, a polypeptide molecule binds cynomolgus monkey TCRβV5-6. In some embodiments, the polypeptide molecule that binds cynomolgus monkey TCRβV5-6 binds a region of SEQ ID NO: 2231. In some embodiments, a polypeptide molecule binds rhesus monkey TCRβV5-6. In some embodiments, the polypeptide molecule that binds rhesus monkey TCRβV5-6 binds a region of SEQ ID NO: 2233. In some embodiments, a polypeptide molecule binds cynomolgus monkey TCRβV6-1. In some embodiments, the polypeptide molecule that binds cynomolgus monkey TCRβV6-1 binds a region of SEQ ID NO: 2410. In some embodiments, a polypeptide molecule binds cynomolgus monkey TCRβV6-2. In some embodiments, the polypeptide molecule that binds cynomolgus monkey TCRβV6-2 binds a region of SEQ ID NO: 2411. In some embodiments, a polypeptide molecule binds cynomolgus monkey TCRβV6-3. In some embodiments, the polypeptide molecule that binds cynomolgus monkey TCRβV6-3 binds a region of SEQ ID NO: 2412. In some embodiments, a polypeptide molecule binds cynomolgus monkey TCRβV6-6. In some embodiments, the polypeptide molecule that binds cynomolgus monkey TCRβV6-6 binds a region of SEQ ID NO: 2413.

In some embodiments, a polypeptide molecule binds human TCRβV6-5. In some embodiments, the polypeptide molecule that binds human TCRβV6-5 binds a region of SEQ ID NO: 223. In some embodiments, a polypeptide molecule binds cynomolgus monkey TCRβV6-5. In some embodiments, the polypeptide molecule that binds cynomolgus monkey TCRβV6-5 binds a region of SEQ ID NO: 2234. In some embodiments, a polypeptide molecule binds rhesus monkey TCRβV6-5. In some embodiments, the polypeptide molecule that binds rhesus monkey TCRβV6-5 binds a region of SEQ ID NO: 2235.

In some embodiments, a polypeptide molecule binds human TCRβV12-3. In some embodiments, the polypeptide molecule that binds human TCRβV12-3 binds a region of SEQ ID NO: 2236 or SEQ ID NO: 2414. In some embodiments, a polypeptide molecule binds cynomolgus monkey TCRβV12-3. In some embodiments, the polypeptide molecule that binds cynomolgus monkey TCRβV12-3 binds a region of SEQ ID NO: 2237 or SEQ ID NO: 2415. In some embodiments, a polypeptide molecule binds rhesus monkey TCRβV12-3. In some embodiments, the polypeptide molecule that binds rhesus monkey TCRβV12-3 binds a region of SEQ ID NO: 2238.

In some embodiments, a polypeptide molecule binds human TCRβV12-4. In some embodiments, the polypeptide molecule that binds human TCRβV12-4 binds a region of SEQ ID NO: 2239. In some embodiments, a polypeptide molecule binds cynomolgus monkey TCRβV12-4. In some embodiments, the polypeptide molecule that binds cynomolgus monkey TCRβV12-4 binds a region of SEQ ID NO: 2240. In some embodiments, a polypeptide molecule binds rhesus monkey TCRβV12-4. In some embodiments, the polypeptide molecule that binds rhesus monkey TCRβV12-4 binds a region of SEQ ID NO: 2241.

In some embodiments, a polypeptide molecule binds human TCRβV20-1. In some embodiments, the polypeptide molecule that binds human TCRβV20-1 binds a region of SEQ ID NO: 2242. In some embodiments, a polypeptide molecule binds cynomolgus monkey TCRβV20-1. In some embodiments, the polypeptide molecule that binds cynomolgus monkey TCRβV20-1 binds a region of SEQ ID NO: 2243. In some embodiments, a polypeptide molecule binds rhesus monkey TCRβV20-1. In some embodiments, the polypeptide molecule that binds rhesus monkey TCRβV20-1 binds a region of SEQ ID NO: 2244.

In some embodiments, the polypeptide molecule comprises a TCRβV-binding domain and the TCRβV-binding domain is an immunoglobulin molecule. In some embodiments, the immunoglobulin molecule is an antibody or antibody fragment. In some embodiments, the TCRβV-binding domain is a full-length antibody, e.g., a polyclonal or monoclonal antibody. In some embodiments, the TCRβV-binding domain is an antibody fragment, such as, e.g., a single domain antibody (sdAb, also referred to as a VHH antibody or nanobody) or single-chain variable fragment (scFv). In some embodiments, the TCRβV-binding domain is a chimeric antibody. In some embodiments, the TCRβV-binding domain is a humanized antibody. In some embodiments, the TCRβV-binding domain is a human antibody.

In some embodiments, the polypeptide molecule comprises a TCRβV-binding domain and the TCRβV-binding domain is an antibody fragment. In some embodiments, the antibody fragment is an Fv fragment. In some embodiments, the antibody fragment comprises a Fab fragment. In some embodiments, the antibody fragment is a F(ab′)2. In some embodiments, the antibody fragment is an scFv. In some embodiments, the antibody fragment is Fab′. In some embodiments, the antibody fragment is an scFv-Fc. In some embodiments, the antibody fragment is a sdAb.

In some embodiments, the TCRβV-binding domain comprises: (a) an immunoglobulin heavy chain variable (VH) region comprising a heavy chain complementarity determining region (CDR) 1 (HCDR1), a heavy chain CDR2 (HCDR2), and a heavy chain CDR3 (HCDR3); and (b) an immunoglobulin light chain variable (VL) region comprising a light chain complementarity determining region (CDR) 1 (LCDR1), a light chain CDR2 (LCDR2), and a light chain CDR3 (LCDR3). In some embodiments, the polypeptide molecule comprises a TCRβV-binding domain comprising: (a) a VH region comprising a HCDR1, a HCDR2, and a HCDR3; and (b) a VL region comprising a LCDR1, a LCDR2, and a LCDR3.

Exemplary TCRβV-binding domains are provided in Table 3.

TABLE 3
Exemplary TCRβV-Binding domains

| | | | |
|---|---|---|---|
| TCRβV6-5 | HCDR1 (IMGT) | GYSFTTYY | 114 |
| | HCDR2 (IMGT) | FFPGSGNI | 115 |
| | HCDR3 (IMGT) | AGSYYSYDVLDY | 116 |
| | LCDR1 (IMGT) | QNVGIN | 113 |
| | LCDR2 (IMGT) | SSS | Not Applicable |
| | LCDR3 (IMGT) | QQFKSYPLT | 73 |
| | HCDR1 (Kabat) | TYYIH | 112 |
| | HCDR2 (Kabat) | WFFPGSGNIKYNEKFKG | 69 |
| | HCDR3 (Kabat) | SYYSYDVLDY | 70 |
| | LCDR1 (Kabat) | KASQNVGINVV | 71 |
| | LCDR2 (Kabat) | SSSHRYS | 72 |
| | LCDR3 (Kabat) | QQFKSYPLT | 73 |
| | VH region | QVQLVQSGAEVKKPGSSVKVSC | 66 |
| | | KASGYSFTTYYIHWVRQAPGQG | |
| | | LEWMGWFFPGSGNIKYNEKFKG | |
| | | RVTITADTSTSTAYMELSSLRSE | |
| | | DTAVYYC1141WGQGTTVTVSS | |
| | VL region | DIQMTQSPSFLSASVGDRVTITC | 67 |
| | | KASQNVGINVVWHQQKPGKAP | |
| | | KALIYSSSHRYSGVPSRFSGSGSG | |
| | | TEFTLTISSLQPEDFATYFCQQFK | |
| | | SYPLTFGQGTKLEIK | |
| TCRβV12- | HCDR1 (IMGT) | GFTFSNFG | 1186 |
| 3/4 | HCDR2 (IMGT) | ISSGSSTI | 1197 |
| | HCDR3 (IMGT) | ARRGEGAMDY | 1201 |
| | LCDR1 (IMGT) | SSVNY | 1182 |
| | LCDR2 (IMGT) | YTS | |
| | LCDR3 (IMGT) | QQFTSSPFT | 1157 |
| | HCDR1 (Kabat) | NFGMH | 1160 |
| | HCDR2 (Kabat) | YISSGSSTIYYADTLKG | 1171 |
| | HCDR3 (Kabat) | RGEGAMDY | 1180 |
| | LCDR1 (Kabat) | RASSSVNYTY | 1148 |
| | LCDR2 (Kabat) | YTSNLAP | 1156 |
| | LCDR3 (Kabat) | QQFTSSPFT | 1157 |
| | VH region | EVQLVESGGGLVQPGGSLRLSC | 1113 |
| | | AASGFTFSNFGMHWVRQAPGKG | |
| | | LEWVSYISSGSSTIYYADTLKGR | |
| | | FTISRDNSKNTLYLQMNSLRAED | |
| | | TAVYYCARRGEGAMDYWGQGT | |
| | | TVTVSS | |
| | VL region | DNQLTQSPSFLSASVGDRVTITC | 1100 |
| | | RASSSVNYIYWYQQKPGKAPKL | |
| | | LIYYTSNLAPGVPSRFSGSGSGNE | |
| | | YTLTISSLQPEDFATYYCQQFTSS | |
| | | PFTFGQGTKLEIK | |
| TCRβV20-1 | HCDR1 (IMGT) | GFNIKSAY | 2245 |
| | HCDR2 (IMGT) | IDPATGKT | 2246 |
| | HCDR3 (IMGT) | TRSLNWDYGLDY | 2247 |
| | LCDR1 (IMGT) | KSVSILGTHL | 2248 |
| | LCDR2 (IMGT) | AAS | |
| | LCDR3 (IMGT) | QQSIEDPWT | 2249 |
| | HCDR1 (Kabat) | SAYMH | 2250 |
| | HCDR2 (Kabat) | RIDPATGKTKYAPKFQA | 2251 |
| | HCDR3 (Kabat) | SLNWDYGLDY | 2252 |
| | LCDR1 (Kabat) | RASKSVSILGTHLIH | 2253 |
| | LCDR2 (Kabat) | AASNLES | 2254 |
| | LCDR3 (Kabat) | QQSIEDPWT | 2249 |
| | VH region | EVQLQQSVADLVRPGASLKLSC | 2255 |
| | | TASGFNIKSAYMHWVIQRPDQG | |
| | | PECLGRIDPATGKTKYAPKFQAK | |
| | | ATITADTSSNTAYLQLSSLTSEDT | |
| | | AIYYCTRSLNWDYGLDYWGQG | |
| | | TSVTVSS | |
| | VL region | DIVLTQSPASLAVSLGQRATISCR | 2256 |
| | | ASKSVSILGTHLIHWYQQKPGQP | |
| | | PKLLIYAASNLESGVPARFSGSGS | |
| | | ETVFTLNIHPVEEEDAATYFCQQ | |
| | | SIEDPWTFGGGTKLGIK | |
| TCRβV5-5/6 | HCDR1 (IMGT) | GFSLTAYG | 2257 |
| | HCDR2 (IMGT) | IWGDGNT | 2258 |
| | HCDR3 (IMGT) | ARDRVTATLYAMDY | 2259 |
| | LCDR1 (IMGT) | QGISNY | 2260 |
| | LCDR2 (IMGT) | YTS | |
| | LCDR3 (IMGT) | QQYSKLPRT | 2261 |
| | HCDR1 (Kabat) | AYGVN | 2262 |
| | HCDR2 (Kabat) | MIWGDGNTDYNSALKS | 2263 |
| | HCDR3 (Kabat) | DRVTATLYAMDY | 2264 |
| | LCDR1 (Kabat) | SASQGISNYLN | 2265 |
| | LCDR2 (Kabat) | YTSSLHS | 2266 |
| | LCDR3 (Kabat) | QQYSKLPRT | 2261 |
| | VH region | QVTLKESGPVLVKPTETLTLTCT | 2267 |
| | | VSGFSLTAYGVNWVRQPPGKAL | |
| | | EWLGMIWGDGNTDYNSALKSR | |
| | | LTISKDNSKSQVVLTMTNMDPV | |
| | | DTATYYCARDRVTATLYAMDY | |
| | | WGQGTLVTVSS | |
| | VL region | DIQMTQSPSSLSASVGDRVTITCS | 2268 |
| | | ASQGISNYLNWYQQKPGKAVKL | |
| | | LIYYTSSLHSGVPSRFSGSGSGTD | |
| | | YTLTISSLQPEDFATYYCQQYSK | |
| | | LPRTFGGGTKVEIK | |

In some embodiments, the polypeptide molecule comprises a TCRβV5-5/6-binding domain. In some embodiments, the TCRβV5-5/6-binding domain comprises an amino acid sequence with at least about 75%, 80%, 85%, 90%, 91%, 92%, 93%, 94%, 95%, 96%, 97%, 98%, 99%, or 100% identity to SEQ ID NO: 2295. In some embodiments, the TCRβV5-5/6 binding domain comprises an amino acid sequence of SEQ ID NO: 2295. In some embodiments, the TCRβV5-5/6-binding domain consists of an amino acid sequence of SEQ ID NO: 2295. In some embodiments, the TCRβV5-5/6-binding domain comprises an scFv.

In some embodiments, the polypeptide molecule comprises a TCRβV6-5-binding domain. In some embodiments, the TCRβV6-5-binding domain comprises an amino acid sequence with at least about 75%, 80%, 85%, 90%, 91%, 92%, 93%, 94%, 95%, 96%, 97%, 98%, 99%, or 100% identity to SEQ ID NO: 149. In some embodiments, the TCRβV6-5-binding domain comprises an amino acid sequence of SEQ ID NO: 149. In some embodiments, the TCRβV6-5-binding domain consists of an amino acid sequence of SEQ ID NO: 149. In some embodiments, the TCRβV6-5-binding domain comprises an scFv.

In some embodiments, the polypeptide molecule comprises a TCRβV12-3/4 binding domain. In some embodiments, the TCRβV12-3/4-binding domain comprises an amino acid sequence with at least about 75%, 80%, 85%, 90%, 91%, 92%, 93%, 94%, 95%, 96%, 97%, 98%, 99%, or 100% identity to SEQ ID NO: 1203. In some embodiments, the TCRβV12-3/4 binding domain comprises an amino acid sequence of SEQ ID NO: 1203. In some embodiments, the TCRβV12-3/4 binding domain consists of an amino acid sequence of SEQ ID NO: 1203. In some embodiments, the TCRβV12-3/4-binding domain comprises an scFv.

In some embodiments, the polypeptide molecule comprises a TCRβV20-1 binding domain. In some embodiments, the TCRβV20-1-binding domain comprises an amino acid sequence with at least about 75%, 80%, 85%, 90%, 91%, 92%, 93%, 94%, 95%, 96%, 97%, 98%, 99%, or 100% identity to SEQ ID NO: 2296. In some embodiments, the TCRβV20-1 binding domain comprises an amino acid sequence of SEQ ID NO: 2296. In some embodiments, the TCRβV20-1 binding domain consists of an amino acid sequence of SEQ ID NO: 2296. In some embodiments, the TCRβV20-1-binding domain comprises an scFv.

TCRβV6-5-binding Domains

In some embodiments, the polypeptide molecule comprises a TCRβV6-5-binding domain. In some embodiments, the TCRβV6-5-binding domain comprises a Kabat HCDR1 that comprises the amino acid sequence of X₃₁X₃₂YIH, wherein X₃₁ is selected from the group consisting of: T, L, H, Y, Q, K, F, S, A, D, V, I, R, W, E, G, N, and P and X₃₂ is selected from the group consisting of Y, F, M, W, L, R, T, G, I, V, A, E, Q, P, H, D, N, and K. In some embodiments, X₃₁ is selected from the group consisting of T and L. In some embodiments, X₃₁ is L. In some embodiments, X₃₂ is selected from the group consisting of Y, F, M, W, L, R, and T. In some embodiments, X₃₂ is selected from the group consisting of M, L, and R. In some embodiments, X₃₂ is W. In some embodiments, the Kabat HCDR1 of the TCRβV6-5-binding domain does not comprise the amino acid sequence of any one of SEQ ID NOs: 112 and 2416-2424.

In some embodiments, the polypeptide molecule comprises a TCRβV6-5-binding domain. In some embodiments, the TCRβV6-5-binding domain comprises a Kabat HCDR2 that comprises the amino acid sequence of X₅₀X₅₁X₅₂X₅₃X₅₄X₅₅X₅₆X₅₇X₅₈X₅₉Y₆₀X₆₁X₆₂K₆₃F₆₄K₆₅G₆₆, wherein X₅₀ is selected from the group consisting of: W, E, D, R, V, H, G, Q, S, M, N, Y, I, L, A, T, P, and F; X₅₁ is selected from the group consisting of F, E, H, M, K, Q, Y, L, N, V, T, A, R, G, S, D, W, I, and P; X₅₂ is selected from the group consisting of: F, Y, R, S, A, K, W, N, M, H, G, Q, D, P, V, L, E, T, and I; X₅₃ is selected from the group consisting of: P, G, N, A, I, V, L, T, Y, S, Q, K, F, R, D, E, and W; X₅₄ is selected from the group consisting of G, R, K, W, L, F, Y, Q, H, I, M, S, D, P, T, and V; X₅₅ is selected from the group consisting of S, T, G, W, H, N, Y, L, A, F, Q, K, V, M, D, E, R, and P; X₅₆ is selected from the group consisting of: G, Q, R, S, W, A, H, M, N, Y, E, L, T, D, K, I, V, and P; X₅₇ is selected from the group consisting of N, K, R, T, G, H, V, Y, M, L, Q, I, D, A, E, F, W, S, and P; X₅₈ is selected from the group consisting of: I, V, Y, L, A, Q, K, T, M, E, S, N, R, F, D, P, and W; X₅₉ is selected from the group consisting of: K, N, Q, M, L, H, F, S, A, V, T, W, E, G, P, D, and R; X₆₁ is selected from the group consisting of N, V, S, H, P, T, D, E, W, G, M, Q, F, K, I, Y, and L; and X₆₂ is selected from the group consisting of E, P, Q, I, N, W, H, F, K, A, V, Y, M, L, S, D, R, T, and G. In some embodiments, X₅₀ is W. In some embodiments, X₅₁ is F. In some embodiments, X₅₂ is F. In some embodiments, X₅₃ is P. In some embodiments, X₅₄ is selected from the group consisting of G and R. In some embodiments, X₅₄ is R. In some embodiments, X₅₅ is S. In some embodiments, X₅₆ is G. In some embodiments, X₅₇ is N. In some embodiments, X₅₈ is selected from the group consisting of I, V, Y, L, A, Q, K, T, and M. In some embodiments, X₅₈ is selected from the group consisting of L, A, Q, K, and M. In some embodiments, X₅₉ is K. In some embodiments, X₆₁ is selected from the group consisting of N, V, S, H, P, T, and D. In some embodiments, X₆₁ is selected from the group consisting of V, S, H, P, T, and D. In some embodiments, X₆₂ is E. In some embodiments, the Kabat HCDR2 of the TCRβV6-5-binding domain does not comprise the amino acid sequence of any one of SEQ ID NOs: 69 and 2425-2446.

In some embodiments, the polypeptide molecule comprises a TCRβV6-5-binding domain. In some embodiments, the TCRβV6-5-binding domain comprises a Kabat HCDR3 that comprises the amino acid sequence of X₉₉X₁₀₀X₁₀₁X₁₀₂X₁₀₃X₁₀₄X₁₀₅X₁₀₆X₁₀₇X₁₀₈, wherein X₉₉ is selected from the group consisting of S, A, P, K, D, E, H, G, L, W, V, Y, I, N, M, R, and F; X₁₀₀ is selected from the group consisting of Y, V, F, I, L, M, T, W, H, D, Q, A, S, E, K, P, N, and R; X₁₀₁ is selected from the group consisting of: Y, W, F, V, H, M, P, Q, S, T, E, N, L, K, D, I, and A; X₁₀₂ is selected from the group consisting of S, A, T, G, N, L, M, V, Y, H, Q, W, I, D, R, E, and F; X₁₀₃ is selected from the group consisting of: Y, F, P, M, K, W, S, V, H, L, R, D, A, E, I, Q, and N; X₁₀₄ is selected from the group consisting of D, N, S, G, L, T, K, Q, H, P, R, E, W, F, and V; X₁₀₅ is selected from the group consisting of V, A, N, Q, T, W, E, P, Y, M, K, H, D, S, F, G, L, and R; X₁₀₆ is selected from the group consisting of: L, I, M, V, Y, A, P, R, T, Q, H, G, K, N, E, D, and S; X₁₀₇ is selected from the group consisting of: D, V, S, Q, I, H, E, R, T, M, Y, G, N, A, F, P, K, W, and L; and X₁₀₈ is selected from the group consisting of: Y, D, V, S, Q, I, H, E, R, T, M, G, N, A, F, P, K, W, and L. In some embodiments, X₉₉ is S. In some embodiments, X₁₀₀ is Y. In some embodiments, X₁₀₁ is Y. In some embodiments, X₁₀₂ is S. In some embodiments, X₁₀₃ is selected from the group consisting of Y and F. In some embodiments, X₁₀₃ is F. In some embodiments, X₁₀₄ is D. In some embodiments, X₁₀₅ is V. In some embodiments, X₁₀₆ is L. In some embodiments, X₁₀₇ is D. In some embodiments, X₁₀s is Y. In some embodiments, the Kabat HCDR3 of the TCRβV6-5-binding domain does not comprise the amino acid sequence of SEQ ID NO: 70 or SEQ ID NO: 2447.

In some embodiments, the polypeptide molecule comprises a TCRβV6-5-binding domain. In some embodiments, the TCRβV6-5-binding domain comprises a Kabat LCDR1 that comprises the amino acid sequence of K₂₄A₂₅S₂₆Q₂₇Z₂₈Z₂₉Z₃₀Z₃₁Z₃₂Z₃₃Z₃₄, wherein Z₂₈ is selected from the group consisting of: N, H, P, T, S, M, G, A, V, W, D, I, F, R, Y, Q, E, and L; Z₂₉ is selected from the group consisting of V, G, A, L, M, S, P, F, Q, T, D, K, W, Y, H, R and E; Z₃₀ is selected from the group consisting of: G, W, A, L, Q, N, R, M, P, D, K, H, I, V, S, E, and F; Z₃₁ is selected from the group consisting of I, E, Q, G, H, S, W, Y, M, N, P, F, A, V, K, D, and L; Z₃₂ is selected from the group consisting of: N, S, H, A, K, Q, L, D, M, T, E, Y, V, F, W, R and P; Z₃₃ is selected from the group consisting of V, D, E, N, L, G, A, T, S, Q, H, F, R, K, W, P, and Y; and Z₃₄ is selected from the group consisting of: V, T, N, I, K, P, H, M, F, Q, G, Y, R, W, L, D, A, and S. In some embodiments, Z₂₈ is N. In some embodiments, Z₂₉ is selected from the group consisting of V and G. In some embodiments, Z₂₉ is G. In some embodiments, Z₃₀ is G. In some embodiments, Z₃₁ is I. In some embodiments, Z₃₂ is selected from the group consisting of N and R. In some embodiments, Z₃₂ is N. In some embodiments, Z₃₂ is R. In some embodiments, Z₃₃ is selected from the group consisting of V and D. In some embodiments, Z₃₃ is D. In some embodiments, Z₃₄ is V. In some embodiments, the Kabat LCDR1 of the TCRβV6-5-binding domain does not comprise the amino acid sequence of any one of SEQ ID NOs: 71 and 2448-2456.

In some embodiments, the polypeptide molecule comprises a TCRβV6-5-binding domain. In some embodiments, the TCRβV6-5-binding domain comprises a Kabat LCDR2 that comprises the amino acid sequence of Z₅₀SZ₅₂Z₅₃Z₅₄Z₅₅Z₅₆, wherein Z₅₀ is selected from the group consisting of: S, V, A, G, H, L, T, M, F, Q, E, P, K, W, and Y; Z₅₂ is selected from the group consisting of S, W, Y, G, T, M, F, N, L, A, V, Q, I, E, D, H, P, and K; Z₅₃ is selected from the group consisting of H, M, A, Y, L, N, E, V, G, Q, F, K, P, W, and R; Z₅₄ is selected from the group consisting of: R, V, N, P, K, T, W, A, M, L, I, H, Q, F, and D; Z₅₅ is selected from the group consisting of Y, W, M, F, A, S, E, G, V, P, D, N, R, Q, T, H; and Z₅₆ is selected from the group consisting of: S, R, K, Q, Y, W, F, H, I, V, T, C, N, G, D, M and P. In some embodiments, Z₅₀ is S. In some embodiments, Z₅₂ is S. In some embodiments, Z₅₃ is H. In some embodiments, Z₅₄ is R. In some embodiments, Z₅₅ is Y. In some embodiments, Z₅₆ is selected from the group consisting of S and R. In some embodiments, Z₅₆ is R. In some embodiments, the Kabat LCDR2 of the TCRβV6-5-binding domain does not comprise the amino acid sequence of SEQ ID NO: 72 or SEQ ID NO: 2457.

In some embodiments, the polypeptide molecule comprises a TCRβV6-5-binding domain. In some embodiments, the TCRβV6-5-binding domain comprises a Kabat LCDR3 that comprises the amino acid sequence of Z₈₉Z₉₀Z₉₁Z₉₂Z₉₃Z₉₄Z₉₅Z₉₆Z₉₇, wherein Z₈₉ is selected from the group consisting of: Q, Y, K, E, V, A, N, I, G, W, S, R, L, and H; Z₉₀ is selected from the group consisting of Q, M, H, E, N, L, D, G, V, R, I, Y, W, K, S and P; Z₉₁ is selected from the group consisting of F, S, T, A, M, H, L, E, Y, W, N, R, Q, I, P, V, G, K, and D; Z₉₂ is selected from the group consisting of: K, G, I, R, S, V, Y, F, W, M, A, Q, T, H, E, L, D, N, and P; Z₉₃ is selected from the group consisting of S, F, R, A, L, M, K, D, W, N, V, Q, T, I, and G; Z₉₄ is selected from the group consisting of: Y, L, V, I, A, N, Q, R, E, G, P, S, K, D, W, and F; Z₉₅ is selected from the group consisting of P, N, A, G, S, H, V, D, K, T, L, E, M, Q, R, F, and W; Z₉₆ is selected from the group consisting of L, I, V, M, E, Y, F, N, A, G, H, R, S, W, Q, D, P, and T; and Z₉₇ is selected from the group consisting of: T, G, A, S, R, Q, V, W, E, L, I, H, M, F, N, D, and P. In some embodiments, Z₈₉ is Q. In some embodiments, Z₉₀ is Q. In some embodiments, Z₉₁ is F. In some embodiments, Z₉₂ is K. In some embodiments, Z₉₃ is selected from the group consisting of S, F, R, A, and L. In some embodiments, Z₉₃ is selected from the group consisting of F, R, A, and L. In some embodiments, Z₉₄ is Y. In some embodiments, Z₉₅ is selected from the group consisting of P and N. In some embodiments, Z₉₅ is N. In some embodiments, Z₉₆ is selected from the group consisting of L and I. In some embodiments, Z₉₆ is I. In some embodiments, Z₉₇ is selected from the group consisting of T, G, and A. In some embodiments, Z₉₇ is selected from the group consisting of G and A. In some embodiments, Z₅₆ is R. In some embodiments, the Kabat LCDR3 of the TCRβV6-5-binding domain does not comprise the amino acid sequence of SEQ ID NO: 73.

In some embodiments, the polypeptide molecule comprises a TCRβV6-5-binding domain. In some embodiments, the TCRβV6-5-binding domain comprises:
a Kabat HCDR1 that comprises the amino acid sequence of X₃₁X₃₂YIH, wherein X₃₁ is selected from the group consisting of: T and L; and X₃₂ is selected from the group consisting of Y, F, M, W, L, R, and T;
a Kabat HCDR2 that comprises the amino acid sequence of W₅₀F₅₁F₅₂P₅₃X₅₄S₅₅G₅₆N₅₇X₅₈K₅₉Y₆₀X₆₁E₆₂K₆₃F₆₄K₆₅G₆₆, wherein X₅₄ is selected from the group consisting of G and R; X₅₈ is selected from the group consisting of: I, V, Y, L, A, Q, K, T, and M; and X₆₁ is selected from the group consisting of N, V, S, H, P, T, and D;
a Kabat HCDR3 that comprises the amino acid sequence of S₉₉Y₁₀₀Y₁₀₁S₁₀₂F₁₀₃D₁₀₄V₁₀₅L₁₀₆D₁₀₇Y₁₀₈;
a Kabat LCDR1 that comprises the amino acid sequence of K₂₄A₂₅S₂₆Q27N₂₈Z₂₉G₃₀I₃₁Z₃₂Z₃₃V₃₄, wherein Z₂₉ is selected from the group consisting of V and G; Z₃₂ is selected from the group consisting of N and R; and Z₃₃ is selected from the group consisting of V and D;
a Kabat LCDR2 that comprises the amino acid sequence of S₅₀S₅₁S₅₂H₅₃R₅₄Y₅₅Z₅₆, wherein Z₅₆ is selected from the group consisting of: S and R;
a Kabat LCDR3 that comprises the amino acid sequence of Q₈₉Q₉₀F₉₁K₉₂Z₉₃Y₉₄Z₉₅L₉₆Z₉₇, wherein Z₉₃ is selected from the group consisting of S, F, R, A, and L; Z₉₅ is selected from the group consisting of P and N; Z₉₆ is selected from the group consisting of L and I; and Z₉₇ is selected from the group consisting of T, G, and A; wherein the polypeptide molecule does not comprise SEQ ID NOs: 69-73, 112, and 2416-2457.

In some embodiments, the polypeptide molecule comprises a TCRβV6-5-binding domain. In some embodiments, the TCRβV6-5-binding domain comprises an IMGT HCDR1 that comprises the amino acid sequence of G₂₆X₂₇X₂₈X₂₉X₃₀X₃₁X₃₂Y₃₃, wherein X₂₇ is selected from the group consisting of: Y, P, E, H, V, K, S, Q, T, L, N, F, M, I, R, G, W, and D; X₂₈ is selected from the group consisting of S, K, A, P, T, V, N, Q, L, E, I, H, M, G, Y, and W; X₂₉ is selected from the group consisting of: F, I, M, Q, L, W, P, V, S, Y, N, A, D, T, E, H, K, G, and R; X₃₀ is selected from the group consisting of T, V, L, Y, W, Q, I, E, R, G, N, F, K, M, S, H, A, and P; X₃₁ is selected from the group consisting of: T, L, H, Y, Q, K, F, S, A, D, V, I, R, W, E, G, N, and P; and X₃₂ is selected from the group consisting of Y, F, M, W, L, R, T, G, I, V, A, E, Q, P, H, D, N, and K. In some embodiments, X₂₇ is Y. In some embodiments, X₂₈ is selected from the group consisting of S, K, A, P, T, and V. In some embodiments, X₂₈ is selected from the group consisting of K, A, P, and V. In some embodiments, X₂₉ is F. In some embodiments, X₃₀ is selected from the group consisting of T, V, L, Y, W, and Q. In some embodiments, X₃₀ is selected from the group consisting of V, L, Y, W, and Q. In some embodiments, X₃₁ is selected from the group consisting of T and L. In some embodiments, X₃₁ is L. In some embodiments, X₃₂ is selected from the group consisting of Y, F, M, W, L, R, and T. In some embodiments, X₃₂ is selected from the group consisting of M, L, and R. In some embodiments, the IMGT HCDR1 of the TCRβV6-5-binding domain does not comprise the amino acid sequence of any one of SEQ ID NOs: 114 and 2458-2476.

In some embodiments, the polypeptide molecule comprises a TCRβV6-5-binding domain. In some embodiments, the TCRβV6-5-binding domain comprises an IMGT HCDR2 that comprises the amino acid sequence of X₅₁X₅₂X₅₃X₅₄X₅₅X₅₆X₅₇X₅₈, wherein X₅₁ is selected from the group consisting of F, E, H, M, K, Q, Y, L, N, V, T, A, R, G, S, D, W, I, and P; X₅₂ is selected from the group consisting of: F, Y, R, S, A, K, W, N, M, H, G, Q, D, P, V, L, E, T, and I; X₅₃ is selected from the group consisting of: P, G, N, A, I, V, L, T, Y, S, Q, K, F, R, D, E, and W; X₅₄ is selected from the group consisting of G, R, K, W, L, F, Y, Q, H, I, M, S, D, P, T, and V; X₅₅ is selected from the group consisting of: S, T, G, W, H, N, Y, L, A, F, Q, K, V, M, D, E, R, and P; X₅₆ is selected from the group consisting of: G, Q, R, S, W, A, H, M, N, Y, E, L, T, D, K, I, V, and P; X₅₇ is selected from the group consisting of N, K, R, T, G, H, V, Y, M, L, Q, I, D, A, E, F, W, S, and P; and X₅₈ is selected from the group consisting of: I, V, Y, L, A, Q, K, T, M, E, S, N, R, F, D, P, and W. In some embodiments, X₅₁ is F. In some embodiments, X₅₂ is F. In some embodiments, X₅₃ is P. In some embodiments, X₅₄ is selected from the group consisting of G and R. In some embodiments, X₅₄ is R. In some embodiments, X₅₅ is S. In some embodiments, X₅₆ is G. In some embodiments, X₅₇ is N. In some embodiments, X₅₈ is selected from the group consisting of I, V, Y, L, A, Q, K, T, and M. In some embodiments, X₅₈ is selected from the group consisting of L, A, Q, K, and M. In some embodiments, the IMGT HCDR2 of the TCRβV6-5-binding domain does not comprise the amino acid sequence of any one of SEQ ID NOs: 115 and 2477-2498.

In some embodiments, the polypeptide molecule comprises a TCRβV6-5-binding domain. In some embodiments, the TCRβV6-5-binding domain comprises an IMGT HCDR3 that comprises the amino acid sequence of A₉₇X₉₈X₉₉X₁₀₀X₁₀₁X₁₀₂X₁₀₃X₁₀₄X₁₀₅X₁₀₆X₁₀₇X₁₀₈, wherein X₉₈ is selected from the group consisting of: G, A, V, P, M, I, D, Q, N, F, L, W, C, and R; X₉₉ is selected from the group consisting of S, A, P, K, D, E, H, G, L, W, V, Y, I, N, M, R, and F; X₁₀₀ is selected from the group consisting of: Y, V, F, I, L, M, T, W, H, D, Q, A, S, E, K, P, N, and R; X₁₀₁ is selected from the group consisting of: Y, W, F, V, H, M, P, Q, S, T, E, N, L, K, D, I, and A; X₁₀₂ is selected from the group consisting of S, A, T, G, N, L, M, V, Y, H, Q, W, I, D, R, E, and F; X₁₀₃ is selected from the group consisting of: Y, F, P, M, K, W, S, V, H, L, R, D, A, E, I, Q, and N; X₁₀₄ is selected from the group consisting of D, N, S, G, L, T, K, Q, H, P, R, E, W, F, and V; X₁₀₅ is selected from the group consisting of V, A, N, Q, T, W, E, P, Y, M, K, H, D, S, F, G, L, and R; X₁₀₆ is selected from the group consisting of: L, I, M, V, Y, A, P, R, T, Q, H, G, K, N, E, D, and S; X₁₀₇ is selected from the group consisting of: D, V, S, Q, I, H, E, R, T, M, Y, G, N, A, F, P, K, W, and L; and X₁₀₈ is selected from the group consisting of: Y, D, V, S, Q, I, H, E, R, T, M, G, N, A, F, P, K, W, and L. wherein X₉₈ is selected from the group consisting of G and A; X₉₉ is S; X₁₀₀ is Y; X₁₀₁ is Y; X₁₀₂ is S; X₁₀₃ is selected from the group consisting of Y and F; X₁₀₄ is D; X₁₀₅ is V; X₁₀₆ is L; X₁₀₇ is D; or X₁₀₈ is Y. In some embodiments, X₉₈ is selected from the group consisting of G and A. In some embodiments, X₉₈ is A. In some embodiments, X₁₀₃ is F. In some embodiments, the IMGT HCDR3 of the TCRβV6-5-binding domain does not comprise the amino acid sequence of SEQ ID NO: 116 or SEQ ID NO: 2499.

In some embodiments, the polypeptide molecule comprises a TCRβV6-5-binding domain. In some embodiments, the TCRβV6-5-binding domain comprises an IMGT LCDR1 that comprises the amino acid sequence of Q₂₇Z₂₈Z₂₉Z₃₀Z₃₁Z₃₂, wherein Z₂₈ is selected from the group consisting of: N, H, P, T, S, M, G, A, V, W, D, I, F, R, Y, Q, E, and L; Z₂₉ is selected from the group consisting of V, G, A, L, M, S, P, F, Q, T, D, K, W, Y, H, R and E; Z₃₀ is selected from the group consisting of: G, W, A, L, Q, N, R, M, P, D, K, H, I, V, S, E, and F; Z₃₁ is selected from the group consisting of I, E, Q, G, H, S, W, Y, M, N, P, F, A, V, K, D, and L; and Z₃₂ is selected from the group consisting of: N, S, H, A, K, Q, L, D, M, T, E, Y, V, F, W, R and P. In some embodiments, Z₂₈ is N. In some embodiments, Z₂₉ is selected from the group consisting of V and G. In some embodiments, Z₂₉ is G. In some embodiments, Z₃₀ is G. In some embodiments, Z₃₁ is I. In some embodiments, Z₃₂ is N. In some embodiments, Z₃₂ is R. In some embodiments, the IMGT LCDR1 of the TCRβV6-5-binding domain does not comprise the amino acid sequence of any one of SEQ ID NOs: 113 and 2500-2508.

In some embodiments, the polypeptide molecule comprises a TCRβV6-5-binding domain. In some embodiments, the TCRβV6-5-binding domain comprises an IMGT LCDR2 that comprises the amino acid sequence of Z₅₀SZ₅₂, wherein Z₅₀ is selected from the group consisting of: S, V, A, G, H, L, T, M, F, Q, E, P, K, W, and Y; and Z₅₂ is selected from the group consisting of S, W, Y, G, T, M, F, N, L, A, V, Q, I, E, D, H, P, and K. In some embodiments, Z₅₀ is S. In some embodiments, Z₅₂ is S. In some embodiments, Z₃₂ is R. In some embodiments, the IMGT LCDR2 of the TCRβV6-5-binding domain does not comprise the amino acid sequence of SSS.

In some embodiments, the polypeptide molecule comprises a TCRβV6-5-binding domain. In some embodiments, the TCRβV6-5-binding domain comprises an IMGT LCDR3 that comprises the amino acid sequence of Z₈₉Z₉₀Z₉₁Z₉₂Z₉₃Z₉₄Z₉₅Z₉₆Z₉₇, wherein Z₈₉ is selected from the group consisting of: Q, Y, K, E, V, A, N, I, G, W, S, R, L, and H; Z₉₀ is selected from the group consisting of Q, M, H, E, N, L, D, G, V, R, I, Y, W, K, S, and P; Z₉₁ is selected from the group consisting of F, S, T, A, M, H, L, E, Y, W, N, R, Q, I, P, V, G, K, and D; Z₉₂ is selected from the group consisting of: K, G, I, R, S, V, Y, F, W, M, A, Q, T, H, E, L, D, N, and P; Z₉₃ is selected from the group consisting of S, F, R, A, L, M, K, D, W, N, V, Q, T, I, and G; Z₉₄ is selected from the group consisting of: Y, L, V, I, A, N, Q, R, E, G, P, S, K, D, W, and F; Z₉₅ is selected from the group consisting of P, N, A, G, S, H, V, D, K, T, L, E, M, Q, R, F, and W; Z₉₆ is selected from the group consisting of L, I, V, M, E, Y, F, N, A, G, H, R, S, W, Q, D, P, and T; and Z₉₇ is selected from the group consisting of: T, G, A, S, R, Q, V, W, E, L, I, H, M, F, N, D, and P. In some embodiments, Z₈₉ is Q. In some embodiments, Z₉₀ is Q. In some embodiments, Z₉₁ is F. In some embodiments, Z₉₂ is K. In some embodiments, Z₉₃ is selected from the group consisting of S, F, R, A, and L. In some embodiments, Z₉₃ is selected from the group consisting of F, R, A, and L. In some embodiments, Z₉₄ is Y. In some embodiments, Z₉₅ is selected from the group consisting of P and N. In some embodiments, Z₉₅ is N. In some embodiments, Z₉₆ is selected from the group consisting of L and I. In some embodiments, Z₉₆ is I. In some embodiments, Z₉₇ is selected from the group consisting of T, G, and A. In some embodiments, Z₉₇ is selected from the group consisting of G and A. In some embodiments, Z₃₂ is R. In some embodiments, the IMGT LCDR3 of the TCRβV6-5-binding domain does not comprise the amino acid sequence of SEQ ID NO: 73.

In some embodiments, the polypeptide molecule comprises a TCRβV6-5-binding domain. In some embodiments, the TCRβV6-5-binding domain comprises:
an IMGT HCDR1 that comprises the amino acid sequence of G₂₆Y₂₇X₂₈F₂₉X₃₀X₃₁X₃₂Y₃₃, wherein X₂₈ is selected from the group consisting of S, K, A, P, T, and V; X₃₀ is selected from the group consisting of T, V, L, Y, W, and Q; X₃₁ is selected from the group consisting of: T and L; and X₃₂ is selected from the group consisting of Y, F, M, W, L, R, and T;
an IMGT HCDR2 that comprises the amino acid sequence of F₅₁F₅₂P₅₃X₅₄S₅₅G₅₆N₅₇X₅₈, wherein X₅₄ is selected from the group consisting of G and R; and X₅₈ is selected from the group consisting of: I, V, Y, L, A, Q, K, T, and M;
an IMGT HCDR3 that comprises the amino acid sequence of A₉₇X₉₈S₉₉Y₁₀₀Y₁₀₁S₁₀₂X₁₀₃D₁₀₄V₁₀₅L₁₀₆D₁₀₇Y₁₀₈, wherein X₉₈ is selected from the group consisting of: G and A; and wherein X₁₀₃ is selected from the group consisting of Y and F;
an IMGT LCDR1 that comprises the amino acid sequence of Q₂₇N₂₈Z₂₉G₃₀I₃₁Z₃₂, wherein Z₂₉ is selected from the group consisting of V and G; and wherein Z₃₂ is selected from the group consisting of N and R;
an IMGT LCDR2 that comprises the amino acid sequence of S₅₀S₅₁S₅₂;
an IMGT LCDR3 that comprises the amino acid sequence of Q₈₉Q₉₀F₉₁K₉₂Z₉₃Y₉₄Z₉₅Z₉₆Z₉₇, wherein Z₉₃ is selected from the group consisting of S, F, R, A, and L; Z₉₅ is selected from the group consisting of P and N; Z₉₆ is selected from the group consisting of L and I; and Z₉₇ is selected from the group consisting of: T, G, and A; and wherein the polypeptide molecule does not comprise the amino acid sequence of SEQ ID NOs: 73, 113-116, and 2458-2508, and SSS.

In some embodiments, the TCRβV6-5-binding domain comprises: a Kabat HCDR1 that comprises the amino acid sequence of LYYIH (SEQ ID NO: 136); a Kabat HCDR2 that comprises the amino acid sequence of WFFPGSGNIKYNEKFKG (SEQ ID NO: 69); a Kabat HCDR3 that comprises the amino acid sequence of SYYSFDVLDY (SEQ ID NO: 137); a Kabat LCDR1 that comprises the amino acid sequence of KASQNVGIRVV (SEQ ID NO: 134); a Kabat LCDR2 that comprises the amino acid sequence of SSSHRYS (SEQ ID NO: 72); and a Kabat LCDR3 that comprises the amino acid sequence of QQFKSYPIT (SEQ ID NO: 135). In some embodiments, the HCDR1, HCDR2, HCDR3, LCDR1, LCDR2, and/or LCDR3 has 1, 2, or 3 mutations.

In some embodiments, the TCRβV6-5-binding domain comprises: a Kabat HCDR1 that comprises the amino acid sequence of TYYIH (SEQ ID NO: 112); a Kabat HCDR2 that comprises the amino acid sequence of WFFPRSGNIKYNEKFKG (SEQ ID NO: 142); a Kabat HCDR3 that comprises the amino acid sequence of SYYSFDVLDY (SEQ ID NO: 137); a Kabat LCDR1 that comprises the amino acid sequence of KASQNVGIRVV (SEQ ID NO: 134); a Kabat LCDR2 that comprises the amino acid sequence of SSSHRYS (SEQ ID NO: 72); and a Kabat LCDR3 that comprises the amino acid sequence of QQFKFYPIT (SEQ ID NO: 141). In some embodiments, the HCDR1, HCDR2, HCDR3, LCDR1, LCDR2, and/or LCDR3 has 1, 2, or 3 mutations.

In some embodiments, the TCRβV6-5-binding domain comprises: a Kabat HCDR1 that comprises the amino acid sequence of TYYIH (SEQ ID NO: 112); a Kabat HCDR2 that comprises the amino acid sequence of WFFPGSGNIKYNEKFKG (SEQ ID NO: 69); a Kabat HCDR3 that comprises the amino acid sequence of SYYSFDVLDY (SEQ ID NO: 137); a Kabat LCDR1 that comprises the amino acid sequence of KASQNVGIRVV (SEQ ID NO: 134); a Kabat LCDR2 that comprises the amino acid sequence of SSSHRYR (SEQ ID NO: 144); and a Kabat LCDR3 that comprises the amino acid sequence of QQFKSYPIT (SEQ ID NO: 135). In some embodiments, the HCDR1, HCDR2, HCDR3, LCDR1, LCDR2, and/or LCDR3 has 1, 2, or 3 mutations.

In some embodiments, the TCRβV6-5-binding domain comprises: a Kabat HCDR1 that comprises the amino acid sequence of TYYIH (SEQ ID NO: 112); a Kabat HCDR2 that comprises the amino acid sequence of WFFPRSGNIKYNEKFKG (SEQ ID NO: 142); a Kabat HCDR3 that comprises the amino acid sequence of SYYSFDVLDY (SEQ ID NO: 137); a Kabat LCDR1 that comprises the amino acid sequence of KASQNVGIRVV (SEQ ID NO: 134); a Kabat LCDR2 that comprises the amino acid sequence of SSSHRYS (SEQ ID NO: 72); and a Kabat LCDR3 that comprises the amino acid sequence of QQFKSYPIT (SEQ ID NO: 135). In some embodiments, the HCDR1, HCDR2, HCDR3, LCDR1, LCDR2, and/or LCDR3 has 1, 2, or 3 mutations.

In some embodiments, the TCRβV6-5-binding domain comprises: a Kabat HCDR1 that comprises the amino acid sequence of LWYIH (SEQ ID NO: 147); a Kabat HCDR2 that comprises the amino acid sequence of WFFPGSGNIKYNEKFKG (SEQ ID NO: 69); a Kabat HCDR3 that comprises the amino acid sequence of SYYSFDVLDY (SEQ ID NO: 137); a Kabat LCDR1 that comprises the amino acid sequence of KASQNVGIRVV (SEQ ID NO: 134); a Kabat LCDR2 that comprises the amino acid sequence of SSSHRYS (SEQ ID NO: 72); and a Kabat LCDR3 that comprises the amino acid sequence of QQFKSYPIT (SEQ ID NO: 135). In some embodiments, the HCDR1, HCDR2, HCDR3, LCDR1, LCDR2, and/or LCDR3 has 1, 2, or 3 mutations.

In some embodiments, the TCRβV6-5-binding domain comprises: a Kabat HCDR1 that comprises the amino acid sequence of LWYIH (SEQ ID NO: 147); a Kabat HCDR2 that comprises the amino acid sequence of WFFPRSGNIKYNEKFKG (SEQ ID NO: 142); a Kabat HCDR3 that comprises the amino acid sequence of SYYSFDVLDY (SEQ ID NO: 137); a Kabat LCDR1 that comprises the amino acid sequence of KASQNVGINVV (SEQ ID NO: 71); a Kabat LCDR2 that comprises the amino acid sequence of SSSHRYK (SEQ ID NO: 128); and a Kabat LCDR3 that comprises the amino acid sequence of QQFKSYPIT (SEQ ID NO: 135). In some embodiments, the HCDR1, HCDR2, HCDR3, LCDR1, LCDR2, and/or LCDR3 has 1, 2, or 3 mutations.

In some embodiments, the TCRβV6-5-binding domain comprises: a Kabat HCDR1 that comprises the amino acid sequence of LYYIH (SEQ ID NO: 136); a Kabat HCDR2 that comprises the amino acid sequence of WFFPRSGNIKYNEKFKG (SEQ ID NO: 142); a Kabat HCDR3 that comprises the amino acid sequence of SYYSFDVLDY (SEQ ID NO: 137); a Kabat LCDR1 that comprises the amino acid sequence of KASQNVGIRVV (SEQ ID NO: 134); a Kabat LCDR2 that comprises the amino acid sequence of SSSHRYK (SEQ ID NO: 128); and a Kabat LCDR3 that comprises the amino acid sequence of QQFKSYPIT (SEQ ID NO: 135). In some embodiments, the HCDR1, HCDR2, HCDR3, LCDR1, LCDR2, and/or LCDR3 has 1, 2, or 3 mutations.

In some embodiments, the TCRβV6-5-binding domain comprises: a Kabat HCDR1 that comprises the amino acid sequence of TYYIH (SEQ ID NO: 112); a Kabat HCDR2 that comprises the amino acid sequence of WFFPRSGNIKYNEKFKG (SEQ ID NO: 142); a Kabat HCDR3 that comprises the amino acid sequence of SYYSFDVLDY (SEQ ID NO: 137); a Kabat LCDR1 that comprises the amino acid sequence of KASQNVGINVV (SEQ ID NO: 71); a Kabat LCDR2 that comprises the amino acid sequence of SSSHRYR (SEQ ID NO: 144); and a Kabat LCDR3 that comprises the amino acid sequence of QQFKSYPIT (SEQ ID NO: 135). In some embodiments, the HCDR1, HCDR2, HCDR3, LCDR1, LCDR2, and/or LCDR3 has 1, 2, or 3 mutations.

In some embodiments, the TCRβV6-5-binding domain comprises: a Kabat HCDR1 that comprises the amino acid sequence of TYYIH (SEQ ID NO: 112); a Kabat HCDR2 that comprises the amino acid sequence of WFFPRSGNIKYNEKFKG (SEQ ID NO: 142); a Kabat HCDR3 that comprises the amino acid sequence of SYYSFDVLDY (SEQ ID NO: 137); a Kabat LCDR1 that comprises the amino acid sequence of KASQNVGIRVV (SEQ ID NO: 134); a Kabat LCDR2 that comprises the amino acid sequence of SSSHRYR (SEQ ID NO: 144); and a Kabat LCDR3 that comprises the amino acid sequence of QQFKSYPIT (SEQ ID NO: 135). In some embodiments, the HCDR1, HCDR2, HCDR3, LCDR1, LCDR2, and/or LCDR3 has 1, 2, or 3 mutations.

In some embodiments, the TCRβV6-5-binding domain comprises: a Kabat HCDR1 that comprises the amino acid sequence of LYYIH (SEQ ID NO: 136); a Kabat HCDR2 that comprises the amino acid sequence of WFFPRSGNIKYNEKFKG (SEQ ID NO: 142); a Kabat HCDR3 that comprises the amino acid sequence of SYYSFDVLDY (SEQ ID NO: 137); a Kabat LCDR1 that comprises the amino acid sequence of KASQNVGIRVV (SEQ ID NO: 134); a Kabat LCDR2 that comprises the amino acid sequence of SSSHRYR (SEQ ID NO: 144); and a Kabat LCDR3 that comprises the amino acid sequence of QQFKSYPIT (SEQ ID NO: 135). In some embodiments, the HCDR1, HCDR2, HCDR3, LCDR1, LCDR2, and/or LCDR3 has 1, 2, or 3 mutations.

In some embodiments, the TCRβV6-5-binding domain comprises: a Kabat HCDR1 that comprises the amino acid sequence of TWYIH (SEQ ID NO: 145); a Kabat HCDR2 that comprises the amino acid sequence of WFFPGSGNIKYNEKFKG (SEQ ID NO: 69); a Kabat HCDR3 that comprises the amino acid sequence of SYYSFDVLDY (SEQ ID NO: 137); a Kabat LCDR1 that comprises the amino acid sequence of KASQNVGIRVV (SEQ ID NO: 134); a Kabat LCDR2 that comprises the amino acid sequence of SSSHRYK (SEQ ID NO: 128); and a Kabat LCDR3 that comprises the amino acid sequence of QQFKFYPIT (SEQ ID NO: 141). In some embodiments, the HCDR1, HCDR2, HCDR3, LCDR1, LCDR2, and/or LCDR3 has 1, 2, or 3 mutations.

In some embodiments, the TCRβV6-5-binding domain comprises: a Kabat HCDR1 that comprises the amino acid sequence of LWYIH (SEQ ID NO: 147); a Kabat HCDR2 that comprises the amino acid sequence of WFFPGSGNIKYNEKFKG (SEQ ID NO: 69); a Kabat HCDR3 that comprises the amino acid sequence of SYYSFDVLDY (SEQ ID NO: 137); a Kabat LCDR1 that comprises the amino acid sequence of KASQNVGINVV (SEQ ID NO: 71); a Kabat LCDR2 that comprises the amino acid sequence of SSSHRYR (SEQ ID NO: 144); and a Kabat LCDR3 that comprises the amino acid sequence of QQFKFYPIT (SEQ ID NO: 141). In some embodiments, the HCDR1, HCDR2, HCDR3, LCDR1, LCDR2, and/or LCDR3 has 1, 2, or 3 mutations.

In some embodiments, the TCRβV6-5-binding domain comprises: a Kabat HCDR1 that comprises the amino acid sequence of LWYIH (SEQ ID NO: 147); a Kabat HCDR2 that comprises the amino acid sequence of WFFPGSGNIKYNEKFKG (SEQ ID NO: 69); a Kabat HCDR3 that comprises the amino acid sequence of SYYSFDVLDY (SEQ ID NO: 137); a Kabat LCDR1 that comprises the amino acid sequence of KASQNVGIRVV (SEQ ID NO: 134); a Kabat LCDR2 that comprises the amino acid sequence of SSSHRYS (SEQ ID NO: 72); and a Kabat LCDR3 that comprises the amino acid sequence of QQFKSYPIT (SEQ ID NO: 135). In some embodiments, the HCDR1, HCDR2, HCDR3, LCDR1, LCDR2, and/or LCDR3 has 1, 2, or 3 mutations.

In some embodiments, the TCRβV6-5-binding domain comprises: a Kabat HCDR1 that comprises the amino acid sequence of TYYIH (SEQ ID NO: 112); a Kabat HCDR2 that comprises the amino acid sequence of WFFPRSGNIKYNEKFKG (SEQ ID NO: 142); a Kabat HCDR3 that comprises the amino acid sequence of SYYSFDVLDY (SEQ ID NO: 137); a Kabat LCDR1 that comprises the amino acid sequence of KASQNVGIRVV (SEQ ID NO: 134); a Kabat LCDR2 that comprises the amino acid sequence of SSSHRYK (SEQ ID NO: 128); and a Kabat LCDR3 that comprises the amino acid sequence of QQFKFYPIT (SEQ ID NO: 141). In some embodiments, the HCDR1, HCDR2, HCDR3, LCDR1, LCDR2, and/or LCDR3 has 1, 2, or 3 mutations.

In some embodiments, the TCRβV6-5-binding domain comprises: a Kabat HCDR1 that comprises the amino acid sequence of LYYIH (SEQ ID NO: 136); a Kabat HCDR2 that comprises the amino acid sequence of WFFPGSGNIKYNEKFKG (SEQ ID NO: 69); a Kabat HCDR3 that comprises the amino acid sequence of SYYSFDVLDY (SEQ ID NO: 137); a Kabat LCDR1 that comprises the amino acid sequence of KASQNVGIRVV (SEQ ID NO: 134); a Kabat LCDR2 that comprises the amino acid sequence of SSSHRYK (SEQ ID NO: 128); and a Kabat LCDR3 that comprises the amino acid sequence of QQFKFYPIT (SEQ ID NO: 141). In some embodiments, the HCDR1, HCDR2, HCDR3, LCDR1, LCDR2, and/or LCDR3 has 1, 2, or 3 mutations.

In some embodiments, the TCRβV6-5-binding domain comprises: a Kabat HCDR1 that comprises the amino acid sequence of LWYIH (SEQ ID NO: 147); a Kabat HCDR2 that comprises the amino acid sequence of WFFPRSGNIKYNEKFKG (SEQ ID NO: 142); a Kabat HCDR3 that comprises the amino acid sequence of SYYSFDVLDY (SEQ ID NO: 137); a Kabat LCDR1 that comprises the amino acid sequence of KASQNVGIRVV (SEQ ID NO: 134); a Kabat LCDR2 that comprises the amino acid sequence of SSSHRYR (SEQ ID NO: 144); and a Kabat LCDR3 that comprises the amino acid sequence of QQFKSYPIT (SEQ ID NO: 135). In some embodiments, the HCDR1, HCDR2, HCDR3, LCDR1, LCDR2, and/or LCDR3 has 1, 2, or 3 mutations.

In some embodiments, the TCRβV6-5-binding domain comprises: a Kabat HCDR1 that comprises the amino acid sequence of LYYIH (SEQ ID NO: 136); a Kabat HCDR2 that comprises the amino acid sequence of WFFPRSGNIKYNEKFKG (SEQ ID NO: 142); a Kabat HCDR3 that comprises the amino acid sequence of SYYSFDVLDY (SEQ ID NO: 137); a Kabat LCDR1 that comprises the amino acid sequence of KASQNVGINVV (SEQ ID NO: 71); a Kabat LCDR2 that comprises the amino acid sequence of SSSHRYR (SEQ ID NO: 144); and a Kabat LCDR3 that comprises the amino acid sequence of QQFKSYPIT (SEQ ID NO: 135). In some embodiments, the HCDR1, HCDR2, HCDR3, LCDR1, LCDR2, and/or LCDR3 has 1, 2, or 3 mutations.

In some embodiments, the TCRβV6-5-binding domain comprises: a Kabat HCDR1 that comprises the amino acid sequence of LWYIH (SEQ ID NO: 147); a Kabat HCDR2 that comprises the amino acid sequence of WFFPRSGNIKYNEKFKG (SEQ ID NO: 142); a Kabat HCDR3 that comprises the amino acid sequence of SYYSFDVLDY (SEQ ID NO: 137); a Kabat LCDR1 that comprises the amino acid sequence of KASQNVGIRVV (SEQ ID NO: 134); a Kabat LCDR2 that comprises the amino acid sequence of SSSHRYR (SEQ ID NO: 144); and a Kabat LCDR3 that comprises the amino acid sequence of QQFKFYPIT (SEQ ID NO: 141). In some embodiments, the HCDR1, HCDR2, HCDR3, LCDR1, LCDR2, and/or LCDR3 has 1, 2, or 3 mutations.

In some embodiments, the TCRβV6-5-binding domain comprises: an IMGT HCDR1 that comprises the amino acid sequence of GYSFTLYY (SEQ ID NO: 139); an IMGT HCDR2 that comprises the amino acid sequence of FFPGSGNI (SEQ ID NO: 115); an IMGT HCDR3 that comprises the amino acid sequence of AASYYSFDVLDY (SEQ ID NO: 140); an IMGT LCDR1 that comprises the amino acid sequence of QNVGIR (SEQ ID NO: 138); an IMGT LCDR2 that comprises the amino acid sequence of SSS; and an LCDR3 that comprises the amino acid sequence of QQFKSYPIT (SEQ ID NO: 135). In some embodiments, the HCDR1, HCDR2, HCDR3, LCDR1, LCDR2, and/or LCDR3 has 1, 2, or 3 mutations.

In some embodiments, the TCRβV6-5-binding domain comprises: an IMGT HCDR1 that comprises the amino acid sequence of GYSFTTYY (SEQ ID NO: 114); an IMGT HCDR2 that comprises the amino acid sequence of FFPRSGNI (SEQ ID NO: 143); an IMGT HCDR3 that comprises the amino acid sequence of AASYYSFDVLDY (SEQ ID NO: 140); an IMGT LCDR1 that comprises the amino acid sequence of QNVGIR (SEQ ID NO: 138); an IMGT LCDR2 that comprises the amino acid sequence of SSS; and an LCDR3 that comprises the amino acid sequence of QQFKFYPIT (SEQ ID NO: 141). In some embodiments, the HCDR1, HCDR2, HCDR3, LCDR1, LCDR2, and/or LCDR3 has 1, 2, or 3 mutations.

In some embodiments, the TCRβV6-5-binding domain comprises: an IMGT HCDR1 that comprises the amino acid sequence of GYSFTTYY (SEQ ID NO: 114); an IMGT HCDR2 that comprises the amino acid sequence of FFPGSGNI (SEQ ID NO: 115); an IMGT HCDR3 that comprises the amino acid sequence of AASYYSFDVLDY (SEQ ID NO: 140); an IMGT LCDR1 that comprises the amino acid sequence of QNVGIR (SEQ ID NO: 138); an IMGT LCDR2 that comprises the amino acid sequence of SSS; and an LCDR3 that comprises the amino acid sequence of QQFKSYPIT (SEQ ID NO: 135). In some embodiments, the HCDR1, HCDR2, HCDR3, LCDR1, LCDR2, and/or LCDR3 has 1, 2, or 3 mutations.

In some embodiments, the TCRβV6-5-binding domain comprises: an IMGT HCDR1 that comprises the amino acid sequence of GYSFTTYY (SEQ ID NO: 114); an IMGT HCDR2 that comprises the amino acid sequence of FFPRSGNI (SEQ ID NO: 143); an IMGT HCDR3 that comprises the amino acid sequence of AASYYSFDVLDY (SEQ ID NO: 140); an IMGT LCDR1 that comprises the amino acid sequence of QNVGIR (SEQ ID NO: 138); an IMGT LCDR2 that comprises the amino acid sequence of SSS; and an LCDR3 that comprises the amino acid sequence of QQFKSYPIT (SEQ ID NO: 135). In some embodiments, the HCDR1, HCDR2, HCDR3, LCDR1, LCDR2, and/or LCDR3 has 1, 2, or 3 mutations.

In some embodiments, the TCRβV6-5-binding domain comprises: an IMGT HCDR1 that comprises the amino acid sequence of GYSFTLWY (SEQ ID NO: 148); an IMGT HCDR2 that comprises the amino acid sequence of FFPGSGNI (SEQ ID NO: 115); an IMGT HCDR3 that comprises the amino acid sequence of AASYYSFDVLDY (SEQ ID NO: 140); an IMGT LCDR1 that comprises the amino acid sequence of QNVGIR (SEQ ID NO: 138); an IMGT LCDR2 that comprises the amino acid sequence of SSS; and an LCDR3 that comprises the amino acid sequence of QQFKSYPIT (SEQ ID NO: 135). In some embodiments, the HCDR1, HCDR2, HCDR3, LCDR1, LCDR2, and/or LCDR3 has 1, 2, or 3 mutations.

In some embodiments, the TCRβV6-5-binding domain comprises: an IMGT HCDR1 that comprises the amino acid sequence of GYSFTLWY (SEQ ID NO: 148); an IMGT HCDR2 that comprises the amino acid sequence of FFPRSGNI (SEQ ID NO: 143); an IMGT HCDR3 that comprises the amino acid sequence of AASYYSFDVLDY (SEQ ID NO: 140); an IMGT LCDR1 that comprises the amino acid sequence of QNVGIN (SEQ ID NO: 113); an IMGT LCDR2 that comprises the amino acid sequence of SSS; and an LCDR3 that comprises the amino acid sequence of QQFKSYPIT (SEQ ID NO: 135). In some embodiments, the HCDR1, HCDR2, HCDR3, LCDR1, LCDR2, and/or LCDR3 has 1, 2, or 3 mutations.

In some embodiments, the TCRβV6-5-binding domain comprises: an IMGT HCDR1 that comprises the amino acid sequence of GYSFTLYY (SEQ ID NO: 139); an IMGT HCDR2 that comprises the amino acid sequence of FFPRSGNI (SEQ ID NO: 143); an IMGT HCDR3 that comprises the amino acid sequence of AASYYSFDVLDY (SEQ ID NO: 140); an IMGT LCDR1 that comprises the amino acid sequence of QNVGIR (SEQ ID NO: 138); an IMGT LCDR2 that comprises the amino acid sequence of SSS; and an LCDR3 that comprises the amino acid sequence of QQFKSYPIT (SEQ ID NO: 135). In some embodiments, the HCDR1, HCDR2, HCDR3, LCDR1, LCDR2, and/or LCDR3 has 1, 2, or 3 mutations.

In some embodiments, the TCRβV6-5-binding domain comprises: an IMGT HCDR1 that comprises the amino acid sequence of GYSFTTYY (SEQ ID NO: 114); an IMGT HCDR2 that comprises the amino acid sequence of FFPRSGNI (SEQ ID NO: 143); an IMGT HCDR3 that comprises the amino acid sequence of AASYYSFDVLDY (SEQ ID NO: 140); an IMGT LCDR1 that comprises the amino acid sequence of QNVGIN (SEQ ID NO: 113); an IMGT LCDR2 that comprises the amino acid sequence of SSS; and an LCDR3 that comprises the amino acid sequence of QQFKSYPIT (SEQ ID NO: 135). In some embodiments, the HCDR1, HCDR2, HCDR3, LCDR1, LCDR2, and/or LCDR3 has 1, 2, or 3 mutations.

In some embodiments, the TCRβV6-5-binding domain comprises: an IMGT HCDR1 that comprises the amino acid sequence of GYSFTTYY (SEQ ID NO: 114); an IMGT HCDR2 that comprises the amino acid sequence of FFPRSGNI (SEQ ID NO: 143); an IMGT HCDR3 that comprises the amino acid sequence of AASYYSFDVLDY (SEQ ID NO: 140); an IMGT LCDR1 that comprises the amino acid sequence of QNVGIR (SEQ ID NO: 138); an IMGT LCDR2 that comprises the amino acid sequence of SSS; and an LCDR3 that comprises the amino acid sequence of QQFKSYPIT (SEQ ID NO: 135). In some embodiments, the HCDR1, HCDR2, HCDR3, LCDR1, LCDR2, and/or LCDR3 has 1, 2, or 3 mutations.

In some embodiments, the TCRβV6-5-binding domain comprises: an IMGT HCDR1 that comprises the amino acid sequence of GYSFTLYY (SEQ ID NO: 139); an IMGT HCDR2 that comprises the amino acid sequence of FFPRSGNI (SEQ ID NO: 143); an IMGT HCDR3 that comprises the amino acid sequence of AASYYSFDVLDY (SEQ ID NO: 140); an IMGT LCDR1 that comprises the amino acid sequence of QNVGIR (SEQ ID NO: 138); an IMGT LCDR2 that comprises the amino acid sequence of SSS; and an LCDR3 that comprises the amino acid sequence of QQFKSYPIT (SEQ ID NO: 135). In some embodiments, the HCDR1, HCDR2, HCDR3, LCDR1, LCDR2, and/or LCDR3 has 1, 2, or 3 mutations.

In some embodiments, the TCRβV6-5-binding domain comprises: an IMGT HCDR1 that comprises the amino acid sequence of GYSFTTWY (SEQ ID NO: 146); an IMGT HCDR2 that comprises the amino acid sequence of FFPGSGNI (SEQ ID NO: 115); an IMGT HCDR3 that comprises the amino acid sequence of AASYYSFDVLDY (SEQ ID NO: 140); an IMGT LCDR1 that comprises the amino acid sequence of QNVGIR (SEQ ID NO: 138); an IMGT LCDR2 that comprises the amino acid sequence of SSS; and an LCDR3 that comprises the amino acid sequence of QQFKFYPIT (SEQ ID NO: 141). In some embodiments, the HCDR1, HCDR2, HCDR3, LCDR1, LCDR2, and/or LCDR3 has 1, 2, or 3 mutations.

In some embodiments, the TCRβV6-5-binding domain comprises: an IMGT HCDR1 that comprises the amino acid sequence of GYSFTLWY (SEQ ID NO: 148); an IMGT HCDR2 that comprises the amino acid sequence of FFPGSGNI (SEQ ID NO: 115); an IMGT HCDR3 that comprises the amino acid sequence of AASYYSFDVLDY (SEQ ID NO: 140); an IMGT LCDR1 that comprises the amino acid sequence of QNVGIR (SEQ ID NO: 138); an IMGT LCDR2 that comprises the amino acid sequence of SSS; and an LCDR3 that comprises the amino acid sequence of QQFKFYPIT (SEQ ID NO: 141). In some embodiments, the HCDR1, HCDR2, HCDR3, LCDR1, LCDR2, and/or LCDR3 has 1, 2, or 3 mutations.

In some embodiments, the TCRβV6-5-binding domain comprises: an IMGT HCDR1 that comprises the amino acid sequence of GYSFTLWY (SEQ ID NO: 148); an IMGT HCDR2 that comprises the amino acid sequence of FFPGSGNI (SEQ ID NO: 115); an IMGT HCDR3 that comprises the amino acid sequence of AASYYSFDVLDY (SEQ ID NO: 140); an IMGT LCDR1 that comprises the amino acid sequence of QNVGIR (SEQ ID NO: 138); an IMGT LCDR2 that comprises the amino acid sequence of SSS; and an LCDR3 that comprises the amino acid sequence of QQFKSYPIT (SEQ ID NO: 135). In some embodiments, the HCDR1, HCDR2, HCDR3, LCDR1, LCDR2, and/or LCDR3 has 1, 2, or 3 mutations.

In some embodiments, the TCRβV6-5-binding domain comprises: an IMGT HCDR1 that comprises the amino acid sequence of GYSFTTYY (SEQ ID NO: 114); an IMGT HCDR2 that comprises the amino acid sequence of FFPRSGNI (SEQ ID NO: 143); an IMGT HCDR3 that comprises the amino acid sequence of AASYYSFDVLDY (SEQ ID NO: 140); an IMGT LCDR1 that comprises the amino acid sequence of QNVGIR (SEQ ID NO: 138); an IMGT LCDR2 that comprises the amino acid sequence of SSS; and an LCDR3 that comprises the amino acid sequence of QQFKFYPIT (SEQ ID NO: 141). In some embodiments, the HCDR1, HCDR2, HCDR3, LCDR1, LCDR2, and/or LCDR3 has 1, 2, or 3 mutations.

In some embodiments, the TCRβV6-5-binding domain comprises: an IMGT HCDR1 that comprises the amino acid sequence of GYSFTLYY (SEQ ID NO: 139); an IMGT HCDR2 that comprises the amino acid sequence of FFPGSGNI (SEQ ID NO: 115); an IMGT HCDR3 that comprises the amino acid sequence of AASYYSFDVLDY (SEQ ID NO: 140); an IMGT LCDR1 that comprises the amino acid sequence of QNVGIR (SEQ ID NO: 138); an IMGT LCDR2 that comprises the amino acid sequence of SSS; and an LCDR3 that comprises the amino acid sequence of QQFKFYPIT (SEQ ID NO: 141). In some embodiments, the HCDR1, HCDR2, HCDR3, LCDR1, LCDR2, and/or LCDR3 has 1, 2, or 3 mutations.

In some embodiments, the TCRβV6-5-binding domain comprises: an IMGT HCDR1 that comprises the amino acid sequence of GYSFTLWY (SEQ ID NO: 148); an IMGT HCDR2 that comprises the amino acid sequence of FFPRSGNI (SEQ ID NO: 143); an IMGT HCDR3 that comprises the amino acid sequence of AASYYSFDVLDY (SEQ ID NO: 140); an IMGT LCDR1 that comprises the amino acid sequence of QNVGIR (SEQ ID NO: 138); an IMGT LCDR2 that comprises the amino acid sequence of SSS; and an LCDR3 that comprises the amino acid sequence of QQFKSYPIT (SEQ ID NO: 135). In some embodiments, the HCDR1, HCDR2, HCDR3, LCDR1, LCDR2, and/or LCDR3 has 1, 2, or 3 mutations.

In some embodiments, the TCRβV6-5-binding domain comprises: an IMGT HCDR1 that comprises the amino acid sequence of GYSFTLYY (SEQ ID NO: 139); an IMGT HCDR2 that comprises the amino acid sequence of FFPRSGNI (SEQ ID NO: 143); an IMGT HCDR3 that comprises the amino acid sequence of AASYYSFDVLDY (SEQ ID NO: 140); an IMGT LCDR1 that comprises the amino acid sequence of QNVGIR (SEQ ID NO: 138); an IMGT LCDR2 that comprises the amino acid sequence of SSS; and an LCDR3 that comprises the amino acid sequence of QQFKSYPIT (SEQ ID NO: 135). In some embodiments, the HCDR1, HCDR2, HCDR3, LCDR1, LCDR2, and/or LCDR3 has 1, 2, or 3 mutations.

In some embodiments, the TCRβV6-5-binding domain comprises: an IMGT HCDR1 that comprises the amino acid sequence of GYSFTLWY (SEQ ID NO: 148); an IMGT HCDR2 that comprises the amino acid sequence of FFPRSGNI (SEQ ID NO: 143); an IMGT HCDR3 that comprises the amino acid sequence of AASYYSFDVLDY (SEQ ID NO: 140); an IMGT LCDR1 that comprises the amino acid sequence of QNVGIR (SEQ ID NO: 138); an IMGT LCDR2 that comprises the amino acid sequence of SSS; and an LCDR3 that comprises the amino acid sequence of QQFKFYPIT (SEQ ID NO: 141). In some embodiments, the HCDR1, HCDR2, HCDR3, LCDR1, LCDR2, and/or LCDR3 has 1, 2, or 3 mutations.

In some embodiments, the TCRβV6-5-binding domain comprises a VH comprising an amino acid sequence with at least about 85%, 90%, 95%, 96%, 97%, 98%, 99%, or 100% identity to SEQ ID NO: 100; and a VL comprising an amino acid sequence with at least about 85%, 90%, 95%, 96%, 97%, 98%, 99%, or 100% identity to SEQ ID NO: 85. In some embodiments, the TCRβV6-5-binding domain comprises a VH comprising an amino acid sequence of SEQ ID NO: 100; and a VL comprising an amino acid sequence of SEQ ID NO: 85.

In some embodiments, the TCRβV6-5-binding domain comprises a VH comprising an amino acid sequence with at least about 85%, 90%, 95%, 96%, 97%, 98%, 99%, or 100% identity to SEQ ID NO: 101; and a VL comprising an amino acid sequence with at least about 85%, 90%, 95%, 96%, 97%, 98%, 99%, or 100% identity to SEQ ID NO: 86. In some embodiments, the TCRβV6-5-binding domain comprises a VH comprising an amino acid sequence of SEQ ID NO: 101; and a VL comprising an amino acid sequence of SEQ ID NO: 86.

In some embodiments, the TCRβV6-5-binding domain comprises a VH comprising an amino acid sequence with at least about 85%, 90%, 95%, 96%, 97%, 98%, 99%, or 100% identity to SEQ ID NO: 102; and a VL comprising an amino acid sequence with at least about 85%, 90%, 95%, 96%, 97%, 98%, 99%, or 100% identity to SEQ ID NO: 87. In some embodiments, the TCRβV6-5-binding domain comprises a VH comprising an amino acid sequence of SEQ ID NO: 102; and a VL comprising an amino acid sequence of SEQ ID NO: 87.

In some embodiments, the TCRβV6-5-binding domain comprises a VH comprising an amino acid sequence with at least about 85%, 90%, 95%, 96%, 97%, 98%, 99%, or 100% identity to SEQ ID NO: 101; and a VL comprising an amino acid sequence with at least about 85%, 90%, 95%, 96%, 97%, 98%, 99%, or 100% identity to SEQ ID NO: 85. In some embodiments, the TCRβV6-5-binding domain comprises a VH comprising an amino acid sequence of SEQ ID NO: 101; and a VL comprising an amino acid sequence of SEQ ID NO: 85.

In some embodiments, the polypeptide molecule comprises a VH comprising an amino acid sequence with at least about 85%, 90%, 95%, 96%, 97%, 98%, 99%, or 100% identity to SEQ ID NO: 103; and a VL comprising an amino acid sequence with at least about 85%, 90%, 95%, 96%, 97%, 98%, 99%, or 100% identity to SEQ ID NO: 85. In some embodiments, the TCRβV6-5-binding domain comprises a VH comprising an amino acid sequence of SEQ ID NO: 103; and a VL comprising an amino acid sequence of SEQ ID NO: 85.

In some embodiments, the TCRβV6-5-binding domain comprises a VH comprising an amino acid sequence with at least about 85%, 90%, 95%, 96%, 97%, 98%, 99%, or 100% identity to SEQ ID NO: 104; and a VL comprising an amino acid sequence with at least about 85%, 90%, 95%, 96%, 97%, 98%, 99%, or 100% identity to SEQ ID NO: 88. In some embodiments, the TCRβV6-5-binding domain comprises a VH comprising an amino acid sequence of SEQ ID NO: 104; and a VL comprising an amino acid sequence of SEQ ID NO: 88.

In some embodiments, the TCRβV6-5-binding domain comprises a VH comprising an amino acid sequence with at least about 85%, 90%, 95%, 96%, 97%, 98%, 99%, or 100% identity to SEQ ID NO: 105; and a VL comprising an amino acid sequence with at least about 85%, 90%, 95%, 96%, 97%, 98%, 99%, or 100% identity to SEQ ID NO: 89. In some embodiments, the TCRβV6-5-binding domain comprises a VH comprising an amino acid sequence of SEQ ID NO: 105; and a VL comprising an amino acid sequence of SEQ ID NO: 89.

In some embodiments, the TCRβV6-5-binding domain comprises a VH comprising an amino acid sequence with at least about 85%, 90%, 95%, 96%, 97%, 98%, 99%, or 100% identity to SEQ ID NO: 101; and a VL comprising an amino acid sequence with at least about 85%, 90%, 95%, 96%, 97%, 98%, 99%, or 100% identity to SEQ ID NO: 91. In some embodiments, the TCRβV6-5-binding domain comprises a VH comprising an amino acid sequence of SEQ ID NO: 101; and a VL comprising an amino acid sequence of SEQ ID NO: 91.

In some embodiments, the TCRβV6-5-binding domain comprises a VH comprising an amino acid sequence with at least about 85%, 90%, 95%, 96%, 97%, 98%, 99%, or 100% identity to SEQ ID NO: 106; and a VL comprising an amino acid sequence with at least about 85%, 90%, 95%, 96%, 97%, 98%, 99%, or 100% identity to SEQ ID NO: 87. In some embodiments, the TCRβV6-5-binding domain comprises a VH comprising an amino acid sequence of SEQ ID NO: 106; and a VL comprising an amino acid sequence of SEQ ID NO: 87.

In some embodiments, the TCRβV6-5-binding domain comprises a VH comprising an amino acid sequence with at least about 85%, 90%, 95%, 96%, 97%, 98%, 99%, or 100% identity to SEQ ID NO: 107; and a VL comprising an amino acid sequence with at least about 85%, 90%, 95%, 96%, 97%, 98%, 99%, or 100% identity to SEQ ID NO: 87. In some embodiments, the TCRβV6-5-binding domain comprises a VH comprising an amino acid sequence of SEQ ID NO: 107; and a VL comprising an amino acid sequence of SEQ ID NO: 87.

In some embodiments, the TCRβV6-5-binding domain comprises a VH comprising an amino acid sequence with at least about 85%, 90%, 95%, 96%, 97%, 98%, 99%, or 100% identity to SEQ ID NO: 108; and a VL comprising an amino acid sequence with at least about 85%, 90%, 95%, 96%, 97%, 98%, 99%, or 100% identity to SEQ ID NO: 89. In some embodiments, the TCRβV6-5-binding domain comprises a VH comprising an amino acid sequence of SEQ ID NO: 108; and a VL comprising an amino acid sequence of SEQ ID NO: 89.

In some embodiments, the TCRβV6-5-binding domain comprises a VH comprising an amino acid sequence with at least about 85%, 90%, 95%, 96%, 97%, 98%, 99%, or 100% identity to SEQ ID NO: 109; and a VL comprising an amino acid sequence with at least about 85%, 90%, 95%, 96%, 97%, 98%, 99%, or 100% identity to SEQ ID NO: 90. In some embodiments, the TCRβV6-5-binding domain comprises a VH comprising an amino acid sequence of SEQ ID NO: 109; and a VL comprising an amino acid sequence of SEQ ID NO: 90.

In some embodiments, the TCRβV6-5-binding domain comprises a VH comprising an amino acid sequence with at least about 85%, 90%, 95%, 96%, 97%, 98%, 99%, or 100% identity to SEQ ID NO: 109; and a VL comprising an amino acid sequence with at least about 85%, 90%, 95%, 96%, 97%, 98%, 99%, or 100% identity to SEQ ID NO: 86. In some embodiments, the TCRβV6-5-binding domain comprises a VH comprising an amino acid sequence of SEQ ID NO: 109; and a VL comprising an amino acid sequence of SEQ ID NO: 86.

In some embodiments, the TCRβV6-5-binding domain comprises a VH comprising an amino acid sequence with at least about 85%, 90%, 95%, 96%, 97%, 98%, 99%, or 100% identity to SEQ ID NO: 106; and a VL comprising an amino acid sequence with at least about 85%, 90%, 95%, 96%, 97%, 98%, 99%, or 100% identity to SEQ ID NO: 92. In some embodiments, the TCRβV6-5-binding domain comprises a VH comprising an amino acid sequence of SEQ ID NO: 106; and a VL comprising an amino acid sequence of SEQ ID NO: 92.

In some embodiments, the TCRβV6-5-binding domain comprises a VH comprising an amino acid sequence with at least about 85%, 90%, 95%, 96%, 97%, 98%, 99%, or 100% identity to SEQ ID NO: 110; and a VL comprising an amino acid sequence with at least about 85%, 90%, 95%, 96%, 97%, 98%, 99%, or 100% identity to SEQ ID NO: 92. In some embodiments, the TCRβV6-5-binding domain comprises a VH comprising an amino acid sequence of SEQ ID NO: 110; and a VL comprising an amino acid sequence of SEQ ID NO: 92.

In some embodiments, the TCRβV6-5-binding domain comprises a VH comprising an amino acid sequence with at least about 85%, 90%, 95%, 96%, 97%, 98%, 99%, or 100% identity to SEQ ID NO: 111; and a VL comprising an amino acid sequence with at least about 85%, 90%, 95%, 96%, 97%, 98%, 99%, or 100% identity to SEQ ID NO: 87. In some embodiments, the TCRβV6-5-binding domain comprises a VH comprising an amino acid sequence of SEQ ID NO: 111; and a VL comprising an amino acid sequence of SEQ ID NO: 87.

In some embodiments, the TCRβV6-5-binding domain comprises a VH comprising an amino acid sequence with at least about 85%, 90%, 95%, 96%, 97%, 98%, 99%, or 100% identity to SEQ ID NO: 107; and a VL comprising an amino acid sequence with at least about 85%, 90%, 95%, 96%, 97%, 98%, 99%, or 100% identity to SEQ ID NO: 91. In some embodiments, the TCRβV6-5-binding domain comprises a VH comprising an amino acid sequence of SEQ ID NO: 107; and a VL comprising an amino acid sequence of SEQ ID NO: 91.

In some embodiments, the TCRβV6-5-binding domain comprises a VH comprising an amino acid sequence with at least about 85%, 90%, 95%, 96%, 97%, 98%, 99%, or 100% identity to SEQ ID NO: 111; and a VL comprising an amino acid sequence with at least about 85%, 90%, 95%, 96%, 97%, 98%, 99%, or 100% identity to SEQ ID NO: 93. In some embodiments, the TCRβV6-5-binding domain comprises a VH comprising an amino acid sequence of SEQ ID NO: 111; and a VL comprising an amino acid sequence of SEQ ID NO: 93.

In some embodiments, the TCRβV6-5-binding domain comprises a VH comprising an amino acid sequence with at least about 85%, 90%, 95%, 96%, 97%, 98%, 99%, or 100% identity to SEQ ID NO: 2512; and a VL comprising an amino acid sequence with at least about 85%, 90%, 95%, 96%, 97%, 98%, 99%, or 100% identity to SEQ ID NO: 2514. In some embodiments, the TCRβV6-5-binding domain comprises a VH comprising an amino acid sequence of SEQ ID NO: 2512; and a VL comprising an amino acid sequence of SEQ ID NO: 2514.

In some embodiments, the TCRβV6-5-binding domain comprises a VH comprising an amino acid sequence with at least about 85%, 90%, 95%, 96%, 97%, 98%, 99%, or 100% identity to SEQ ID NO: 2513; and a VL comprising an amino acid sequence with at least about 85%, 90%, 95%, 96%, 97%, 98%, 99%, or 100% identity to SEQ ID NO: 2514. In some embodiments, the TCRβV6-5-binding domain comprises a VH comprising an amino acid sequence of SEQ ID NO: 2513; and a VL comprising an amino acid sequence of SEQ ID NO: 2514.

In some embodiments, the polypeptide molecule comprises a TCRβV6-5-binding domain. In some embodiments, the TCRβV6-5-binding domain is one selected from those disclosed in Table 20. In some embodiments, the TCRβV6-5-binding domain comprises an amino any one or more of the amino acid sequences in Table 20. In some embodiments, the TCRβV6-5-binding domain comprises an amino acid sequence with at least about or at most about 75%, 80%, 85%, 90%, 91%, 92%, 93%, 94%, 95%, 96%, 97%, 98%, 99%, or 100% identity to any one or more of the amino acid sequences in Table 20.

In some embodiments, the polypeptide molecule comprises a TCRβV6-5-binding domain. In some embodiments, the TCRβV6-5-binding domain comprises an amino acid sequence of Table 21. In some embodiments, the TCRβV6-5-binding domain comprises an amino acid sequence with at least about or at most about 80%, 85%, 90%, 95%, 96%, 97%, 98%, 99%, or 100% identity to any one of the amino acid sequences in Table 21. In some embodiments, the TCRβV6-5-binding domain comprises an amino acid sequence with at least about or at most about 75%, 80%, 85%, 90%, 91%, 92%, 93%, 94%, 95%, 96%, 97%, 98%, 99%, or 100% identical to any one of SEQ ID NOs: 149-222. In some embodiments, the TCRβV6-5-binding domain comprises an amino acid sequence of any one of SEQ ID NOs: 149-222. In some embodiments, the TCRβV6-5-binding domain consists of an amino acid sequence of any one of SEQ ID NOs: 149-222. In some embodiments, the polypeptide molecule is an scFv.

In some embodiments, the TCRβV6-5-binding domain is an scFv. In some embodiments, the TCRβV6-5-binding domain comprises an amino acid sequence with at least about or at most about 75%, 80%, 85%, 90%, 91%, 92%, 93%, 94%, 95%, 96%, 97%, 98%, 99%, or 100% identity to SEQ ID NO: 205. In some embodiments, the TCRβV6-5-binding domain comprises an amino acid sequence of SEQ ID NO: 205. In some embodiments, the TCRβV6-5-binding domain consists of an amino acid sequence of SEQ ID NO: 205.

In some embodiments, the TCRβV6-5-binding domain is an scFv. In some embodiments, the TCRβV6-5-binding domain comprises an amino acid sequence with at least about or at most about 75%, 80%, 85%, 90%, 91%, 92%, 93%, 94%, 95%, 96%, 97%, 98%, 99%, or 100% identity to SEQ ID NO: 206. In some embodiments, the TCRβV6-5-binding domain comprises an amino acid sequence of SEQ ID NO: 206. In some embodiments, the TCRβV6-5-binding domain consists of an amino acid sequence of SEQ ID NO: 206.

In some embodiments, the TCRβV6-5-binding domain is an scFv. In some embodiments, the TCRβV6-5-binding domain comprises an amino acid sequence with at least about or at most about 75%, 80%, 85%, 90%, 91%, 92%, 93%, 94%, 95%, 96%, 97%, 98%, 99%, or 100% identity to SEQ ID NO: 207. In some embodiments, the TCRβV6-5-binding domain comprises an amino acid sequence of SEQ ID NO: 207. In some embodiments, the TCRβV6-5-binding domain consists of an amino acid sequence of SEQ ID NO: 207.

In some embodiments, the TCRβV6-5-binding domain is an scFv. In some embodiments, the TCRβV6-5-binding domain comprises an amino acid sequence with at least about or at most about 75%, 80%, 85%, 90%, 91%, 92%, 93%, 94%, 95%, 96%, 97%, 98%, 99%, or 100% identity to SEQ ID NO: 208. In some embodiments, the TCRβV6-5-binding domain comprises an amino acid sequence of SEQ ID NO: 208. In some embodiments, the TCRβV6-5-binding domain consists of an amino acid sequence of SEQ ID NO: 208.

In some embodiments, the TCRβV6-5-binding domain is an scFv. In some embodiments, the TCRβV6-5-binding domain comprises an amino acid sequence with at least about or at most about 75%, 80%, 85%, 90%, 91%, 92%, 93%, 94%, 95%, 96%, 97%, 98%, 99%, or 100% identity to SEQ ID NO: 209. In some embodiments, the TCRβV6-5-binding domain comprises an amino acid sequence of SEQ ID NO: 209. In some embodiments, the TCRβV6-5-binding domain consists of an amino acid sequence of SEQ ID NO: 209.

In some embodiments, the TCRβV6-5-binding domain is an scFv. In some embodiments, the TCRβV6-5-binding domain comprises an amino acid sequence with at least about or at most about 75%, 80%, 85%, 90%, 91%, 92%, 93%, 94%, 95%, 96%, 97%, 98%, 99%, or 100% identity to SEQ ID NO: 210. In some embodiments, the TCRβV6-5-binding domain comprises an amino acid sequence of SEQ ID NO: 210. In some embodiments, the TCRβV6-5-binding domain consists of an amino acid sequence of SEQ ID NO: 210.

In some embodiments, the TCRβV6-5-binding domain is an scFv. In some embodiments, the TCRβV6-5-binding domain comprises an amino acid sequence with at least about or at most about 75%, 80%, 85%, 90%, 91%, 92%, 93%, 94%, 95%, 96%, 97%, 98%, 99%, or 100% identity to SEQ ID NO: 211. In some embodiments, the TCRβV6-5-binding domain comprises an amino acid sequence of SEQ ID NO: 211. In some embodiments, the TCRβV6-5-binding domain consists of an amino acid sequence of SEQ ID NO: 211.

In some embodiments, the TCRβV6-5-binding domain is an scFv. In some embodiments, the TCRβV6-5-binding domain comprises an amino acid sequence with at least about or at most about 75%, 80%, 85%, 90%, 91%, 92%, 93%, 94%, 95%, 96%, 97%, 98%, 99%, or 100% identity to SEQ ID NO: 212. In some embodiments, the TCRβV6-5-binding domain comprises an amino acid sequence of SEQ ID NO: 212. In some embodiments, the TCRβV6-5-binding domain consists of an amino acid sequence of SEQ ID NO: 212.

In some embodiments, the polypeptide molecule comprises a TCRβV6-5-binding domain. In some embodiments, the TCRβV6-5-binding domain comprises a light chain and/or a heavy chain having at least about 75%, 80%, 85%, 90%, 91%, 92%, 93%, 94%, 95%, 96%, 97%, 98%, 99%, or 100% identity to an amino acid sequence of Table 13, Table 14, and Table 17. In some embodiments, the TCRβV6-5-binding domain comprises the HCDR 1-3 and LCDR 1-3 of any of the binding domains disclosed in Table 13, Table 14, and Table 17. In some embodiments, the polypeptide molecule comprises a heavy chain comprising an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to any one of SEQ ID NOs: 576, 582-587, 594-1084, and 1099. In some embodiments, the polypeptide molecule comprises a light chain comprising an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to any one of SEQ ID NOs: 231, 236-574, and 1085-1094. In some embodiments, the polypeptide molecule comprises: (i) a heavy chain comprising an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to any one of SEQ ID NOs: 576, 582-587, 594-1084, and 1099; and (ii) a light chain comprising an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to any one of SEQ ID NOs: 231, 236-574, and 1085-1094. In some embodiments, the polypeptide molecule comprises a Fab that specifically binds to TCRβV6-5.

In some embodiments, the polypeptide molecule comprises a TCRβV6-5-binding domain. In some embodiments, the TCRβV6-5-binding domain comprises a light chain and/or a heavy chain having at least about 75%, 80%, 85%, 90%, 91%, 92%, 93%, 94%, 95%, 96%, 97%, 98%, 99%, or 100% identity to any one of the amino acid sequence in Table 22. In some embodiments, the TCRβV6-5-binding domain comprises the HCDR 1-3 and LCDR 1-3 of any of the binding domains disclosed in Table 22. In some embodiments, the TCRβV6-5-binding domain comprises a heavy chain comprising an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to any one of SEQ ID NOs: 575-1084. In some embodiments, the TCRβV6-5-binding domain comprises a light chain comprising an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to any one of SEQ ID NOs: 230-574. In some embodiments, the TCRβV6-5-binding domain comprises: (i) a heavy chain comprising an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to any one of SEQ ID NOs: 575-1084; and (ii) a light chain comprising an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to any one of SEQ ID NOs: 230-574. In some embodiments, the polypeptide molecule comprises a Fab.

In some embodiments, the TCRβV6-5-binding domain comprises a Fab. In some embodiments, the TCRβV6-5-binding domain comprises: (i) a heavy chain comprising an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to SEQ ID NO: 582; and (ii) a light chain comprising an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to SEQ ID NO: 236.

In some embodiments, the TCRβV6-5-binding domain comprises a Fab. In some embodiments, the TCRβV6-5-binding domain comprises: (i) a heavy chain comprising an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to SEQ ID NO: 583; and (ii) a light chain comprising an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to SEQ ID NO: 237.

In some embodiments, the TCRβV6-5-binding domain comprises a Fab. In some embodiments, the TCRβV6-5-binding domain comprises: (i) a heavy chain comprising an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to SEQ ID NO: 584; and (ii) a light chain comprising an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to SEQ ID NO: 238.

In some embodiments, the TCRβV6-5-binding domain comprises a Fab. In some embodiments, the TCRβV6-5-binding domain comprises: (i) a heavy chain comprising an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to SEQ ID NO: 583; and (ii) a light chain comprising an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to SEQ ID NO: 236.

In some embodiments, the TCRβV6-5-binding domain comprises a Fab. In some embodiments, the TCRβV6-5-binding domain comprises: (i) a heavy chain comprising an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to SEQ ID NO: 585; and (ii) a light chain comprising an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to SEQ ID NO: 236.

In some embodiments, the TCRβV6-5-binding domain comprises a Fab. In some embodiments, the TCRβV6-5-binding domain comprises: (i) a heavy chain comprising an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to SEQ ID NO: 586; and (ii) a light chain comprising an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to SEQ ID NO: 239.

In some embodiments, the TCRβV6-5-binding domain comprises a Fab. In some embodiments, the TCRβV6-5-binding domain comprises: (i) a heavy chain comprising an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to SEQ ID NO: 587; and (ii) a light chain comprising an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to SEQ ID NO: 240.

In some embodiments, the TCRβV6-5-binding domain comprises a Fab. In some embodiments, the TCRβV6-5-binding domain comprises: (i) a heavy chain comprising an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to SEQ ID NO: 583; and (ii) a light chain comprising an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to SEQ ID NO: 241.

In some embodiments, the TCRβV6-5-binding domain specifically binds to a TCRβV6-5 with high affinity. In some embodiments, a TCRβV6-5-binding domain has a dissociation constant of about 10⁻⁵ to about 10⁻¹² moles per liter or molar (M). For example, a TCRβV6-5-binding domain can have a dissociation constant of about 1000 nM, about 900 nM, about 800 nM, about 700 nM, about 600 nM, about 500 nM, about 400 nM, about 300 nM, about 200 nM, about 100 nM, about 75 nM, about 50 nM, about 25 nM, or about 10 nM, including all ranges and subranges therebetween. In some embodiments, a TCRβV6-5-binding domain has a dissociation constant of less than 200 nM.

### TCRβV12-3-Binding Domains

In some embodiments, the polypeptide molecule comprises a TCRβV12-3-binding domain. In some embodiments, the TCRβV12-3-binding domain comprises a Kabat HCDR1 that comprises the amino acid sequence of X₃₁X₃₂G₃₃M₃₄X₃₅, wherein X₃₁ is selected from the group consisting of: N, R, I, Y, V, H, L, T, S, Q, D, F, K, M, A, W, G, E, and P; X₃₂ is selected from the group consisting of F, K, T, Y, V, R, A, H, I, G, M, L, Q, N, E, W, D, and P; and X₃₅ is selected from the group consisting of H, W, F, N, G, S, D, M, A, Y, L, T, I, E, Q, K, and P. In some embodiments, X₃₁ is selected from the group consisting of N, R, I, Y, V, H, L, T, S, Q, and D. In some embodiments, X₃₁ is selected from the group consisting of R and I. In some embodiments, X₃₂ is selected from the group consisting of F, K, T, Y, V, R, A, H, and I. In some embodiments, X₃₂ is T. In some embodiments, X₃₅ is H. In some embodiments, the Kabat HCDR1 of the TCRβV12-3-binding domain does not comprise the amino acid sequence of SEQ ID NO: 1160.

In some embodiments, the polypeptide molecule comprises a TCRβV12-3-binding domain. In some embodiments, the TCRβV12-3-binding domain comprises a Kabat HCDR2 that comprises the amino acid sequence of Y₅₀I₅₁S₅₂X₅₃X₅₄X₅₅X₅₆X₅₇X₅₈X₅₉Y₆₀X₆₁X₆₂X₆₃L₆₄K₆₅G₆₆, wherein X₅₃ is selected from the group consisting of: S, T, G, K, A, L, W, Y, E, D, I, M, V, N, P, F, H, and Q; X₅₄ is selected from the group consisting of G, W, S, Y, T, Q, V, R, H, E, M, K, P, N, F, A, L, I, and D; X₅₅ is selected from the group consisting of S, H, G, Q, A, T, L, M, E, W, N, K, R, Y, D, P, I, and F; X₅₆ is selected from the group consisting of: S, M, T, P, K, R, Q, Y, L, G, N, H, I, D, V, F, W, and E; X₅₇ is selected from the group consisting of T, V, S, L, A, I, P, M, R, K, Q, H, W, F, Y, E, and D; X₅₈ is selected from the group consisting of: I, F, W, Y, V, E, L, M, A, K, R, T, D, N, S, P, H, and G; X₅₉ is selected from the group consisting of: Y, L, I, H, M, F, R, K, V, T, Q, G, P, A, S, W, and D; X₆₁ is selected from the group consisting of A, K, R, D, Q, W, N, S, Y, G, F, E, P, H, T, V, I, L, and M; X₆₂ is selected from the group consisting of D, P, T, E, H, G, R, N, Q, K, I, Y, W, L, F, and V; and X₆₃ is selected from the group consisting of T, I, K, N, H, E, G, M, R, Y, V, F, L, P, S, Q, A, W, and D. In some embodiments, X₅₃ is S. In some embodiments, X₅₄ is selected from the group consisting of G, W, S, Y, T, Q, V, R, H, E, M, K, and P. In some embodiments, X₅₄ is selected from the group consisting of S and T. In some embodiments, X₅₅ is selected from the group consisting of S, H. In some embodiments, X₅₆ is S. In some embodiments, X₅₇ is selected from the group consisting of T and V. In some embodiments, X₅₈ is selected from the group consisting of I, F, W, and Y. In some embodiments, X₅₉ is selected from the group consisting of Y, L, I, and H. In some embodiments, X₅₉ is L. In some embodiments, X₆₁ is selected from the group consisting of A, K, R, D, Q, W, N, S, Y, G, and F. In some embodiments, X₆₂ is selected from the group consisting of D, P, and T. In some embodiments, X₆₃ is selected from the group consisting of T, I, K, N, H, E, G, M, R, Y, V, F, L, and P. In some embodiments, the Kabat HCDR2 of the TCRβV12-3-binding domain does not comprise the amino acid sequence of SEQ ID NO: 1171.

In some embodiments, the polypeptide molecule comprises a TCRβV12-3-binding domain. In some embodiments, the TCRβV12-3-binding domain comprises a Kabat HCDR3 that comprises the amino acid sequence of X₉₉X₁₀₀X₁₀₁X₁₀₂X₁₀₃X₁₀₄X₁₀₅X₁₀₆, wherein X₉₉ is selected from the group consisting of R, I, K, L, P, N, S, Y, V, A, G, T, D, Q, H, M, and W; X₁₀₀ is selected from the group consisting of: G, N, H, S, K, Y, M, R, F, L, D, E, T, I, W, V, and P; X₁₀₁ is selected from the group consisting of: E, H, L, A, T, M, R, S, G, N, Y, W, K, I, P, V, and D; X₁₀₂ is selected from the group consisting of G, W, H, Y, K, R, A, F, N, V, M, S, T, E, L, P, I, and D; X₁₀₃ is selected from the group consisting of: A, S, K, Y, G, T, V, P, R, F, W, N, I, Q, L, H, M, D, and E; X₁₀₄ is selected from the group consisting of M, L, A, P, F, G, E, N, T, S, R, H, Q, W, Y, and D; X₁₀₅ is selected from the group consisting of: D, I, H, Q, Y, K, T, W, L, R, S, E, M, G, P, F, and V; and X₁₀₆ is selected from the group consisting of: Y, N, A, W, V, M, P, T, Q, R, S, H, L, F, G, and D. In some embodiments, X₉₉ is R. In some embodiments, X₁₀₀ is G. In some embodiments, X₁₀₁ is selected from the group consisting of E, H, L, A, T, M, R, S, G, N, Y, W, K, I, and P. In some embodiments, X₁₀₁ is A. In some embodiments, X₁₀₂ is G. In some embodiments, X₁₀₃ is A. In some embodiments, X₁₀₄ is M. In some embodiments, X₁₀₅ is D. In some embodiments, X₁₀₆ is selected from the group consisting of Y, N, A, W, V, M, P, and T. In some embodiments, the Kabat HCDR3 of the TCRβV12-3-binding domain does not comprise the amino acid sequence of SEQ ID NO: 1180.

In some embodiments, the polypeptide molecule comprises a TCRβV12-3-binding domain. In some embodiments, the TCRβV12-3-binding domain comprises a Kabat LCDR1 that comprises the amino acid sequence of R₂₄A₂₅Z₂₆Z₂₇Z₂₈Z₂₉Z₃₀Z₃₁Z₃₂Z₃₃, wherein Z₂₆ is selected from the group consisting of: S, M, V, L, Q, R, H, Y, I, T, F, A, W, N, P, G, D, and K; Z₂₇ is selected from the group consisting of S, K, R, H, Y, N, I, Q, T, G, M, L, F, V, A, W, P, E, and D; Z₂₈ is selected from the group consisting of: S, K, N, R, P, M, Y, A, I, H, V, Q, W, D, F, E, T, and G; Z₂₉ is selected from the group consisting of I, T, L, A, R, M, G, P, Q, Y, F, H, W, N, E, and D; Z₃₀ is selected from the group consisting of: N, V, R, K, M, A, T, L, W, E, Y, Q, S, H, G, P, F, and D; Z₃₁ is selected from the group consisting of Y, F, Y, K, R, H, A, N, M, Q, V, P, G, L, T, E, I, and D; Z₃₂ is selected from the group consisting of: I, A, S, M, L, T, Q, D, P, H, F, N, K, W, Y, and R; and Z₃₃ is selected from the group consisting of Y, D, W, S, L, F, H, T, R, G, P, A, N, V, and I. In some embodiments, Z₂₆ is selected from the group consisting of S, M, V, and L. In some embodiments, Z₂₇ is selected from the group consisting of S, K, R, and H. In some embodiments, Z₂₇ is selected from the group consisting of K and R. In some embodiments, Z₂₈ is selected from the group consisting of S, K, N, R, and P. In some embodiments, Z₂₉ is I. In some embodiments, Z₃₀ is selected from the group consisting of N and V. In some embodiments, Z₃₁ is selected from the group consisting of Y and F. In some embodiments, Z₃₂ is selected from the group consisting of I, A, and S. In some embodiments, Z₃₂ is A. In some embodiments, Z₃₃ is Y. In some embodiments, the Kabat LCDR1 of the TCRβV12-3-binding domain does not comprise the amino acid sequence of SEQ ID NO: 1148.

In some embodiments, the polypeptide molecule comprises a TCRβV12-3-binding domain. In some embodiments, the TCRβV12-3-binding domain comprises a Kabat LCDR2 that comprises the amino acid sequence of Z₄₉Z₅₀Z₅₁Z₅₂Z₅₃Z₅₄Z₅₅, wherein Z₄₉ is selected from the group consisting of: Y, K, Y, F, I, T, H, M, W, L, Q, A, G, P, V, N, E, and D; Z₅₀ is selected from the group consisting of: T, P, S, I, Q, A, W, F, M, N, G, L, Y, R, K, D, E, and H; Z₅₁ is selected from the group consisting of: S, N, K, R, P, D, L, T, V, Y, H, A, Q, W, I, F, C, E, and G; Z₅₂ is selected from the group consisting of N, R, K, A, L, V, F, M, W, G, E, Y, H, D, I, S, and P; Z₅₃ is selected from the group consisting of L, H, K, A, N, Y, R, F, P, V, C, I, W, G, E, D, and T; Z₅₄ is selected from the group consisting of: A, R, V, I, H, F, K, T, P, Q, N, G, E, D, and Y; and Z₅₅ is selected from the group consisting of P, R, T, Q, V, L, W, E, D, G, I, H, S, M, F, N, and Y. In some embodiments, Z₄₉ is selected from the group consisting of Y and K. In some embodiments, Z₅₀ is selected from the group consisting of T and P. In some embodiments, Z₅₁ is selected from the group consisting of S, N, K, R, P, D, and L. In some embodiments, Z₅₂ is selected from the group consisting of N, R, K, A, and L. In some embodiments, Z₅₃ is selected from the group consisting of L and H. In some embodiments, Z₅₄ is selected from the group consisting of A, R, and V. In some embodiments, Z₅₅ is selected from the group consisting of P, R, T, Q, V, and L. In some embodiments, Z₃₃ is Y. In some embodiments, the Kabat LCDR2 of the TCRβV12-3-binding domain does not comprise the amino acid sequence of SEQ ID NO: 1156.

In some embodiments, the polypeptide molecule comprises a TCRβV12-3-binding domain. In some embodiments, the TCRβV12-3-binding domain comprises a Kabat LCDR3 that comprises the amino acid sequence of Z₈₈Z₈₉Z₉₀Z₉₁Z₉₂Z₉₃Z₉₄Z₉₅Z₉₆, wherein Z₈₈ is selected from the group consisting of: Q, E, L, Y, N, M, P, D, S, V, H, T, W, I, F, and R; Z₈₉ is selected from the group consisting of: Q, H, E, K, V, R, T, G, S, A, L, I, F, M, Y, D, and P; Z₉₀ is selected from the group consisting of F, S, L, K, M, Y, V, Q, N, A, R, I, T, H, G, W, D, P, and E; Z₉₁ is selected from the group consisting of T, S, I, H, N, A, K, G, R, Q, Y, M, F, L, V, P, W, E, and D; Z₉₂ is selected from the group consisting of: S, G, R, A, N, Y, M, W, D, T, F, V, K, Q, H, L, I, E, and P; Z₉₃ is selected from the group consisting of S, K, A, F, R, P, H, T, V, M, L, E, N, Q, W, D, and G; Z₉₄ is selected from the group consisting of: P, W, E, I, Y, T, F, H, L, A, S, C, R, K, N, Q, and V; Z₉₅ is selected from the group consisting of F, L, P, M, V, W, A, N, S, I, G, E, H, Q, and R; and Z₉₆ is selected from the group consisting of T, V, S, H, Y, L, F, I, W, R, Q, M, E, K, G, and D. In some embodiments, Z₈₈ is Q. In some embodiments, Z₈₉ is Q. In some embodiments, Z₉₀ is F. In some embodiments, Z₉₁ is T. In some embodiments, Z₉₂ is selected from the group consisting of S, G, and R. In some embodiments, Z₉₃ is selected from the group consisting of S, K, A, F, R, P, and H. In some embodiments, Z₉₄ is P. In some embodiments, Z₉₅ is F. In some embodiments, Z₉₆ is selected from the group consisting of T, V, S, H, and Y. In some embodiments, Z₉₆ is V. In some embodiments, Z₃₃ is Y. In some embodiments, the Kabat LCDR3 of the TCRβV12-3-binding domain does not comprise the amino acid sequence of SEQ ID NO: 1157.

In some embodiments, the polypeptide molecule comprises a TCRβV12-3-binding domain. In some embodiments, the TCRβV12-3-binding domain comprises a Kabat HCDR1 that comprises the amino acid sequence of X₃₁X₃₂G₃₃M₃₄H₃₅, wherein X₃₁ is selected from the group consisting of: N, R, I, Y, V, H, L, T, S, Q, and D; and X₃₂ is selected from the group consisting of F, K, T, Y, V, R, A, H, and I;
a Kabat HCDR2 that comprises the amino acid sequence of Y₅₀I₅₁S₅₂S₅₃X₅₄X₅₅S₅₆X₅₇X₅₈X₅₉Y₆₀X₆₁X₆₂X₆₃L₆₄K₆₅G₆₆, wherein X₅₄ is selected from the group consisting of G, W, S, Y, T, Q, V, R, H, E, M, K, and P; X₅₅ is selected from the group consisting of S and H; X₅₇ is selected from the group consisting of T and V; X₅₈ is selected from the group consisting of: I, F, W, and Y; X₅₉ is selected from the group consisting of: Y, L, I, and H; X₆₁ is selected from the group consisting of A, K, R, D, Q, W, N, S, Y, G, and F; X₆₂ is selected from the group consisting of D, P, and T; and X₆₃ is selected from the group consisting of T, I, K, N, H, E, G, M, R, Y, V, F, L, and P;
a Kabat HCDR3 that comprises the amino acid sequence of R₉₉G₁₀₀X₁₀₁G₁₀₂A₁₀₃M₁₀₄D₁₀₅X₁₀₆, wherein X₁₀₁ is selected from the group consisting of: E, H, L, A, T, M, R, S, G, N, Y, W, K, I, and P; and X₁₀₆ is selected from the group consisting of: Y, N, A, W, V, M, P, and T;
a Kabat LCDR1 that comprises the amino acid sequence of R₂₄A₂₅Z₂₆Z₂₇Z₂₈I₂₉Z₃₀Z₃₁Z₃₂Y₃₃, wherein Z₂₆ is selected from the group consisting of: S, M, V, and L; Z₂₇ is selected from the group consisting of S, K, R, and H; Z₂₈ is selected from the group consisting of: S, K, N, R, and P; Z₃₀ is selected from the group consisting of: N and V; Z₃₁ is selected from the group consisting of Y and F; Z₃₂ is selected from the group consisting of: I, A, and S;
a Kabat LCDR2 that comprises the amino acid sequence of Z₄₉Z₅₀Z₅₁Z₅₂Z₅₃Z₅₄Z₅₅, wherein Z₄₉ is selected from the group consisting of: Y and K; Z₅₀ is selected from the group consisting of: T and P; Z₅₁ is selected from the group consisting of: S, N, K, R, P, D, and L; Z₅₂ is selected from the group consisting of N, R, K, A, and L; Z₅₃ is selected from the group consisting of L and H; Z₅₄ is selected from the group consisting of: A, R, and V; and Z₅₅ is selected from the group consisting of P, R, T, Q, V, and L;
a Kabat LCDR3 that comprises the amino acid sequence of Q₈₈Q₈₉F₉₀T₉₁Z₉₂Z₉₃P₉₄F₉₅Z₉₆, wherein Z₉₂ is selected from the group consisting of: S, G, and R; Z₉₃ is selected from the group consisting of S, K, A, F, R, P, and H; and Z₉₆ is selected from the group consisting of T, V, S, H, and Y.
wherein the polypeptide molecule does not comprise SEQ ID NOs: 1148, 1156, 1157, 1160, 1171, and 1180.

In some embodiments, the polypeptide molecule comprises a TCRβV12-3-binding domain. In some embodiments, the TCRβV12-3-binding domain comprises an IMGT HCDR1 that comprises the amino acid sequence of G₂₆X₂₇X₂₈X₂₉X₃₀X₃₁X₃₂G₃₃, wherein X₂₇ is selected from the group consisting of: F, Y, W, M, I, L, H, V, A, Q, G, T, N, P, S, R, K, and E; X₂₈ is selected from the group consisting of T, K, S, M, N, I, F, H, G, Q, R, W, V, Y, E, A, D, and L; X₂₉ is selected from the group consisting of: F, Y, W, M, I, T, A, E, L, V, S, P, N, Q, H, G, K, R, and D; X₃₀ is selected from the group consisting of S, H, R, Q, W, F, K, N, Y, M, V, G, P, L, E, D, and I; X₃₁ is selected from the group consisting of: N, R, I, Y, V, H, L, T, S, Q, D, F, K, M, A, W, G, E, and P; and X₃₂ is selected from the group consisting of F, K, T, Y, V, R, A, H, I, G, M, L, Q, N, E, W, D, and P. In some embodiments, X₂₇ is F. In some embodiments, X₂₈ is selected from the group consisting of T and K. In some embodiments, X₂₉ is F. In some embodiments, X₃₀ is selected from the group consisting of S and H. In some embodiments, X₃₁ is selected from the group consisting of T N, R, I, Y, V, H, L, T, S, Q, and D. In some embodiments, X₃₁ is selected from the group consisting of R and I. In some embodiments, X₃₂ is selected from the group consisting of F, K, T, Y, V, R, A, H, and I. In some embodiments, X₃₂ is T. In some embodiments, the IMGT HCDR1 of the TCRβV12-3-binding domain does not comprise the amino acid sequence of SEQ ID NO: 1186.

In some embodiments, the polypeptide molecule comprises a TCRβV12-3-binding domain. In some embodiments, the TCRβV12-3-binding domain comprises an IMGT HCDR2 that comprises the amino acid sequence of I₅₁S₅₂X₅₃X₅₄X₅₅X₅₆X₅₇X₅₈, wherein X₅₃ is selected from the group consisting of: S, T, G, K, A, L, W, Y, E, D, I, M, V, N, P, F, H, and Q; X₅₄ is selected from the group consisting of G, W, S, Y, T, Q, V, R, H, E, M, K, P, N, F, A, L, I, and D; X₅₅ is selected from the group consisting of S, H, G, Q, A, T, L, M, E, W, N, K, R, Y, D, P, I, and F; X₅₆ is selected from the group consisting of: S, M, T, P, K, R, Q, Y, L, G, N, H, I, D, V, F, W, and E; X₅₇ is selected from the group consisting of T, V, S, L, A, I, P, M, R, K, Q, H, W, F, Y, E, and D; and X₅₈ is selected from the group consisting of: I, F, W, Y, V, E, L, M, A, K, R, T, D, N, S, P, H, and G. In some embodiments, X₅₃ is S. In some embodiments, X₅₄ is selected from the group consisting of G, W, S, Y, T, Q, V, R, H, E, M, K, and P. In some embodiments, X₅₄ is selected from the group consisting of S and T. In some embodiments, X₅₅ is selected from the group consisting of S and H. In some embodiments, X₅₆ is S. In some embodiments, X₅₇ is selected from the group consisting of T and V. In some embodiments, X₅₈ is selected from the group consisting of I, F, W, and Y. In some embodiments, the IMGT HCDR2 of the TCRβV12-3-binding domain does not comprise the amino acid sequence of SEQ ID NO: 1197.

In some embodiments, the polypeptide molecule comprises a TCRβV12-3-binding domain. In some embodiments, the TCRβV12-3-binding domain comprises an IMGT HCDR3 that comprises the amino acid sequence of A₉₇R₉₈X₉₉X₁₀₀X₁₀₁X₁₀₂X₁₀₃X₁₀₄X₁₀₅X₁₀₆, wherein X₉₉ is selected from the group consisting of R, I, K, L, P, N, S, Y, V, A, G, T, D, Q, H, M, and W; X₁₀₀ is selected from the group consisting of: G, N, H, S, K, Y, M, R, F, L, D, E, T, I, W, V, and P; X₁₀₁ is selected from the group consisting of: E, H, L, A, T, M, R, S, G, N, Y, W, K, I, P, V, and D; X₁₀₂ is selected from the group consisting of G, W, H, Y, K, R, A, F, N, V, M, S, T, E, L, P, I, and D; X₁₀₃ is selected from the group consisting of: A, S, K, Y, G, T, V, P, R, F, W, N, I, Q, L, H, M, D, and E; X₁₀₄ is selected from the group consisting of M, L, A, P, F, G, E, N, T, S, R, H, Q, W, Y, and D; X₁₀₅ is selected from the group consisting of D, I, H, Q, Y, K, T, W, L, R, S, E, M, G, P, F, and V; and X₁₀₆ is selected from the group consisting of: Y, N, A, W, V, M, P, T, Q, R, S, H, L, F, G, and D. In some embodiments, X₉₉ is R. In some embodiments, X₁₀₀ is G. In some embodiments, X₁₀₁ is selected from the group consisting of E, H, L, A, T, M, R, S, G, N, Y, W, K, I, and P. In some embodiments, X₁₀₁ is A. In some embodiments, X₁₀₂ is G. In some embodiments, X₁₀₃ is A. In some embodiments, X₁₀₄ is M. In some embodiments, X₁₀₅ is D. In some embodiments, X₁₀₆ is selected from the group consisting of Y, N, A, W, V, M, P, and T. In some embodiments, the IMGT HCDR3 of the TCRβV12-3-binding domain does not comprise the amino acid sequence of SEQ ID NO: 1201.

In some embodiments, the polypeptide molecule comprises a TCRβV12-3-binding domain. In some embodiments, the TCRβV12-3-binding domain comprises an IMGT LCDR1 that comprises the amino acid sequence of Z₂₇Z₂₈Z₂₉Z₃₀Z₃₁, wherein Z₂₇ is selected from the group consisting of: S, K, R, H, Y, N, I, Q, T, G, M, L, F, V, A, W, P, E, and D; Z₂₈ is selected from the group consisting of: S, K, N, R, P, M, Y, A, I, H, V, Q, W, D, F, E, T, and G; Z₂₉ is selected from the group consisting of I, T, L, A, R, M, G, P, Q, Y, F, H, W, N, E, and D; Z₃₀ is selected from the group consisting of: N, V, R, K, M, A, T, L, W, E, Y, Q, S, H, G, P, F, and D; and Z₃₁ is selected from the group consisting of Y, F, Y, K, R, H, A, N, M, Q, V, P, G, L, T, E, I, and D. In some embodiments, Z₂₇ is selected from the group consisting of S, K, R, and H. In some embodiments, Z₂₇ is selected from the group consisting of K and R. In some embodiments, Z₂₈ is selected from the group consisting of S, K, N, R, and P. In some embodiments, Z₂₉ is I. In some embodiments, Z₃₀ is selected from the group consisting of N and V. In some embodiments, Z₃₁ is selected from the group consisting of Y and F. In some embodiments, the IMGT LCDR1 of the TCRβV12-3-binding domain does not comprise the amino acid sequence of SEQ ID NO: 1182.

In some embodiments, the polypeptide molecule comprises a TCRβV12-3-binding domain. In some embodiments, the TCRβV12-3-binding domain comprises an IMGT LCDR2 that comprises the amino acid sequence of Z₄₉Z₅₀Z₅₁, wherein Z₄₉ is selected from the group consisting of: Y, K, Y, F, I, T, H, M, W, L, Q, A, G, P, V, N, E, and D; Z₅₀ is selected from the group consisting of: T, P, S, I, Q, A, W, F, M, N, G, L, Y, R, K, D, E, and H; and Z₅₁ is selected from the group consisting of S, N, K, R, P, D, L, T, V, Y, H, A, Q, W, I, F, C, E, and G. In some embodiments, Z₄₉ is selected from the group consisting of Y and K. In some embodiments, Z₅₀ is selected from the group consisting of T and P. In some embodiments, Z₅₁ is selected from the group consisting of S, N, K, R, P, D, and L. In some embodiments, the IMGT LCDR2 of the TCRβV12-3-binding domain does not comprise the amino acid sequence of YTS.

In some embodiments, the polypeptide molecule comprises a TCRβV12-3-binding domain. In some embodiments, the TCRβV12-3-binding domain comprises an IMGT LCDR3 that comprises the amino acid sequence of Z₈₈Z₈₉Z₉₀Z₉₁Z₉₂Z₉₃Z₉₄Z₉₅Z₉₆, wherein Z₈₈ is selected from the group consisting of: Q, E, L, Y, N, M, P, D, S, V, H, T, W, I, F, and R; Z₈₉ is selected from the group consisting of: Q, H, E, K, V, R, T, G, S, A, L, I, F, M, Y, D, and P; Z₉₀ is selected from the group consisting of F, S, L, K, M, Y, V, Q, N, A, R, I, T, H, G, W, D, P, and E; Z₉₁ is selected from the group consisting of: T, S, I, H, N, A, K, G, R, Q, Y, M, F, L, V, P, W, E, and D; Z₉₂ is selected from the group consisting of: S, G, R, A, N, Y, M, W, D, T, F, V, K, Q, H, L, I, E, and P; Z₉₃ is selected from the group consisting of S, K, A, F, R, P, H, T, V, M, L, E, N, Q, W, D, and G; Z₉₄ is selected from the group consisting of: P, W, E, I, Y, T, F, H, L, A, S, C, R, K, N, Q, and V; Z₉₅ is selected from the group consisting of F, L, P, M, V, W, A, N, S, I, G, E, H, Q, and R; and Z₉₆ is selected from the group consisting of T, V, S, H, Y, L, F, I, W, R, Q, M, E, K, G, and D. In some embodiments, Z₈₈ is Q. In some embodiments, Z₈₉ is Q. In some embodiments, Z₉₀ is F. In some embodiments, Z₉₁ is T. In some embodiments, Z₉₂ is selected from the group consisting of S, G, and R. In some embodiments, Z₉₃ is selected from the group consisting of S, K, A, F, R, P, and H; Z₉₄ is P. In some embodiments, Z₉₅ is F. In some embodiments, Z₉₆ is selected from the group consisting of T, V, S, H, and Y. In some embodiments, Z₉₆ is V. In some embodiments, the IMGT LCDR3 of the TCRβV12-3-binding domain does not comprise the amino acid sequence of SEQ ID NO: 1157.

In some embodiments, the polypeptide molecule comprises a TCRβV12-3-binding domain. In some embodiments, the TCRβV12-3-binding domain comprises an IMGT HCDR1 that comprises the amino acid sequence of G₂₆F₂₇X₂₈F₂₉X₃₀X₃₁X₃₂G₃₃, wherein X₂₈ is selected from the group consisting of T and K; X₃₀ is selected from the group consisting of S and H; X₃₁ is selected from the group consisting of: N, R, I, Y, V, H, L, T, S, Q, and D; and X₃₂ is selected from the group consisting of F, K, T, Y, V, R, A, H, and I;
an IMGT HCDR2 that comprises the amino acid sequence of I₅₁S₅₂S₅₃X₅₄X₅₅S₅₆X₅₇X₅₈, wherein X₅₄ is selected from the group consisting of G, W, S, Y, T, Q, V, R, H, E, M, K, and P; X₅₅ is selected from the group consisting of S and H; X₅₇ is selected from the group consisting of T and V; and X₅₈ is selected from the group consisting of: I, F, W, and Y;
an IMGT HCDR3 that comprises the amino acid sequence of A₉₇R₉₈R₉₉G₁₀₀X₁₀₁G₁₀₂A₁₀₃M₁₀₄D₁₀₅X₁₀₆, wherein X₁₀₁ is selected from the group consisting of: E, H, L, A, T, M, R, S, G, N, Y, W, K, I, and P; and X₁₀₆ is selected from the group consisting of: Y, N, A, W, V, M, P, and T;
an IMGT LCDR1 that comprises the amino acid sequence of Z₂₇Z₂₈I₂₉Z₃₀Z₃₁, wherein Z₂₇ is selected from the group consisting of: S, K, R, and H; Z₂₈ is selected from the group consisting of: S, K, N, R, and P; Z₃₀ is selected from the group consisting of: N and V; and Z₃₁ is selected from the group consisting of Y and F;
an IMGT LCDR2 that comprises the amino acid sequence of Z₄₉Z₅₀Z₅₁, wherein Z₄₉ is selected from the group consisting of: Y and K; Z₅₀ is selected from the group consisting of: T and P; and Z₅₁ is selected from the group consisting of S, N, K, R, P, D, and L;
an IMGT LCDR3 that comprises the amino acid sequence of Q₈₈Q₈₉F₉₀T₉₁Z₉₂Z₉₃P₉₄F₉₅Z₉₆, wherein Z₉₂ is selected from the group consisting of: S, G, and R; Z₉₃ is selected from the group consisting of S, K, A, F, R, P, and H; and Z₉₆ is selected from the group consisting of T, V, S, H, and Y; and
wherein the polypeptide molecule does not comprise the amino acid sequence of SEQ ID NOs: 1157, 1182, 1186, 1197, and 1201, and YTS.

In some embodiments, the TCRβV12-3-binding domain comprises: a Kabat HCDR1 that comprises the amino acid sequence of IFGMH (SEQ ID NO: 1162); a Kabat HCDR2 that comprises the amino acid sequence of YISSTSSTILYADTLKG (SEQ ID NO: 1173); a Kabat HCDR3 that comprises the amino acid sequence of RGEGAMDY (SEQ ID NO: 1180); a Kabat LCDR1 that comprises the amino acid sequence of RASRSVNYAY (SEQ ID NO: 1151); a Kabat LCDR2 that comprises the amino acid sequence of YTSNLAP (SEQ ID NO: 1156); and a Kabat LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises: a Kabat HCDR1 that comprises the amino acid sequence of IFGMH (SEQ ID NO: 1162); a Kabat HCDR2 that comprises the amino acid sequence of YISSTSSTILYADTLKG (SEQ ID NO: 1173); a Kabat HCDR3 that comprises the amino acid sequence of RGEGAMDY (SEQ ID NO: 1180); a Kabat LCDR1 that comprises the amino acid sequence of RASSSVNYIY (SEQ ID NO: 1148); a Kabat LCDR2 that comprises the amino acid sequence of YTSNLAP (SEQ ID NO: 1156); and a Kabat LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises: a Kabat HCDR1 that comprises the amino acid sequence of RTGMH (SEQ ID NO: 1163); a Kabat HCDR2 that comprises the amino acid sequence of YISSSSSTILYADTLKG (SEQ ID NO: 1174); a Kabat HCDR3 that comprises the amino acid sequence of RGEGAMDY (SEQ ID NO: 1180); a Kabat LCDR1 that comprises the amino acid sequence of RASRSVNYAY (SEQ ID NO: 1151); a Kabat LCDR2 that comprises the amino acid sequence of YTSNLAP (SEQ ID NO: 1156); and a Kabat LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises: a Kabat HCDR1 that comprises the amino acid sequence of IKGMH (SEQ ID NO: 1164); a Kabat HCDR2 that comprises the amino acid sequence of YISSGSSTIYYADTLKG (SEQ ID NO: 1171); a Kabat HCDR3 that comprises the amino acid sequence of RGAGAMDY (SEQ ID NO: 1181); a Kabat LCDR1 that comprises the amino acid sequence of RASKSVNYAY (SEQ ID NO: 1152); a Kabat LCDR2 that comprises the amino acid sequence of YTSNLAP (SEQ ID NO: 1156); and a Kabat LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises: a Kabat HCDR1 that comprises the amino acid sequence of NKGMH (SEQ ID NO: 1165); a Kabat HCDR2 that comprises the amino acid sequence of YISSSSSTIYYADTLKG (SEQ ID NO: 1175); a Kabat HCDR3 that comprises the amino acid sequence of RGEGAMDY (SEQ ID NO: 1180); a Kabat LCDR1 that comprises the amino acid sequence of RASKSVNYIY (SEQ ID NO: 1153); a Kabat LCDR2 that comprises the amino acid sequence of YTSNLAP (SEQ ID NO: 1156); and a Kabat LCDR3 that comprises the amino acid sequence of QQFTSSPFT (SEQ ID NO: 1157). In some embodiments, the HCDR1, HCDR2, HCDR3, LCDR1, LCDR2, and/or LCDR3 has 1, 2, or 3 mutations.

In some embodiments, the TCRβV12-3-binding domain comprises: a Kabat HCDR1 that comprises the amino acid sequence of NFGMH (SEQ ID NO: 1160); a Kabat HCDR2 that comprises the amino acid sequence of YISSWSSTILYADTLKG (SEQ ID NO: 1176); a Kabat HCDR3 that comprises the amino acid sequence of RGAGAMDY (SEQ ID NO: 1181); a Kabat LCDR1 that comprises the amino acid sequence of RASSSVNYAY (SEQ ID NO: 1154); a Kabat LCDR2 that comprises the amino acid sequence of YTSNLAP (SEQ ID NO: 1156); and a Kabat LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises: a Kabat HCDR1 that comprises the amino acid sequence of IFGMH (SEQ ID NO: 1162); a Kabat HCDR2 that comprises the amino acid sequence of YISSWSSTIYYADTLKG (SEQ ID NO: 1177); a Kabat HCDR3 that comprises the amino acid sequence of RGEGAMDY (SEQ ID NO: 1180); a Kabat LCDR1 that comprises the amino acid sequence of RASRSVNYAY (SEQ ID NO: 1151); a Kabat LCDR2 that comprises the amino acid sequence of YTSNLAP (SEQ ID NO: 1156); and a Kabat LCDR3 that comprises the amino acid sequence of QQFTSSPFT (SEQ ID NO: 1157). In some embodiments, the HCDR1, HCDR2, HCDR3, LCDR1, LCDR2, and/or LCDR3 has 1, 2, or 3 mutations.

In some embodiments, the TCRβV12-3-binding domain comprises: a Kabat HCDR1 that comprises the amino acid sequence of RFGMH (SEQ ID NO: 1166); a Kabat HCDR2 that comprises the amino acid sequence of YISSWSSTILYADTLKG (SEQ ID NO: 1176); a Kabat HCDR3 that comprises the amino acid sequence of RGEGAMDY (SEQ ID NO: 1180); a Kabat LCDR1 that comprises the amino acid sequence of RASKSVNYIY (SEQ ID NO: 1153); a Kabat LCDR2 that comprises the amino acid sequence of YTSNLAP (SEQ ID NO: 1156); and a Kabat LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises: a Kabat HCDR1 that comprises the amino acid sequence of RKGMH (SEQ ID NO: 1167); a Kabat HCDR2 that comprises the amino acid sequence of YISSTSSTILYADTLKG (SEQ ID NO: 1173); a Kabat HCDR3 that comprises the amino acid sequence of RGEGAMDY (SEQ ID NO: 1180); a Kabat LCDR1 that comprises the amino acid sequence of RASRSVNYIY (SEQ ID NO: 1155); a Kabat LCDR2 that comprises the amino acid sequence of YTSNLAP (SEQ ID NO: 1156); and a Kabat LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises: a Kabat HCDR1 that comprises the amino acid sequence of IFGMH (SEQ ID NO: 1162); a Kabat HCDR2 that comprises the amino acid sequence of YISSWSSTIYYADTLKG (SEQ ID NO: 1177); a Kabat HCDR3 that comprises the amino acid sequence of RGAGAMDY (SEQ ID NO: 1181); a Kabat LCDR1 that comprises the amino acid sequence of RASSSVNYAY (SEQ ID NO: 1154); a Kabat LCDR2 that comprises the amino acid sequence of YTSNLAP (SEQ ID NO: 1156); and a Kabat LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises: a Kabat HCDR1 that comprises the amino acid sequence of IFGMH (SEQ ID NO: 1162); a Kabat HCDR2 that comprises the amino acid sequence of YISSSSSTILYADTLKG (SEQ ID NO: 1174); a Kabat HCDR3 that comprises the amino acid sequence of RGAGAMDY (SEQ ID NO: 1181); a Kabat LCDR1 that comprises the amino acid sequence of RASSSVNYAY (SEQ ID NO: 1154); a Kabat LCDR2 that comprises the amino acid sequence of YTSNLAP (SEQ ID NO: 1156); and a Kabat LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises: a Kabat HCDR1 that comprises the amino acid sequence of NKGMH (SEQ ID NO: 1165); a Kabat HCDR2 that comprises the amino acid sequence of YISSSSSTILYADTLKG (SEQ ID NO: 1174); a Kabat HCDR3 that comprises the amino acid sequence of RGAGAMDY (SEQ ID NO: 1181); a Kabat LCDR1 that comprises the amino acid sequence of RASSSVNYAY (SEQ ID NO: 1154); a Kabat LCDR2 that comprises the amino acid sequence of YTSNLAP (SEQ ID NO: 1156); and a Kabat LCDR3 that comprises the amino acid sequence of QQFTSSPFT (SEQ ID NO: 1157). In some embodiments, the HCDR1, HCDR2, HCDR3, LCDR1, LCDR2, and/or LCDR3 has 1, 2, or 3 mutations.

In some embodiments, the TCRβV12-3-binding domain comprises: a Kabat HCDR1 that comprises the amino acid sequence of RFGMH (SEQ ID NO: 1166); a Kabat HCDR2 that comprises the amino acid sequence of YISSTSSTIYYADTLKG (SEQ ID NO: 1178); a Kabat HCDR3 that comprises the amino acid sequence of RGEGAMDY (SEQ ID NO: 1180); a Kabat LCDR1 that comprises the amino acid sequence of RASRSVNYAY (SEQ ID NO: 1151); a Kabat LCDR2 that comprises the amino acid sequence of YTSNLAP (SEQ ID NO: 1156); and a Kabat LCDR3 that comprises the amino acid sequence of QQFTSSPFT (SEQ ID NO: 1157). In some embodiments, the HCDR1, HCDR2, HCDR3, LCDR1, LCDR2, and/or LCDR3 has 1, 2, or 3 mutations.

In some embodiments, the TCRβV12-3-binding domain comprises: a Kabat HCDR1 that comprises the amino acid sequence of ITGMH (SEQ ID NO: 1168); a Kabat HCDR2 that comprises the amino acid sequence of YISSTSSTILYADTLKG (SEQ ID NO: 1173); a Kabat HCDR3 that comprises the amino acid sequence of RGAGAMDY (SEQ ID NO: 1181); a Kabat LCDR1 that comprises the amino acid sequence of RASKSVNYIY (SEQ ID NO: 1153); a Kabat LCDR2 that comprises the amino acid sequence of YTSNLAP (SEQ ID NO: 1156); and a Kabat LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises: a Kabat HCDR1 that comprises the amino acid sequence of RKGMH (SEQ ID NO: 1167); a Kabat HCDR2 that comprises the amino acid sequence of YISSGSSTIYYADTLKG (SEQ ID NO: 1171); a Kabat HCDR3 that comprises the amino acid sequence of RGAGAMDY (SEQ ID NO: 1181); a Kabat LCDR1 that comprises the amino acid sequence of RASSSVNYAY (SEQ ID NO: 1154); a Kabat LCDR2 that comprises the amino acid sequence of YTSNLAP (SEQ ID NO: 1156); and a Kabat LCDR3 that comprises the amino acid sequence of QQFTSSPFT (SEQ ID NO: 1157). In some embodiments, the HCDR1, HCDR2, HCDR3, LCDR1, LCDR2, and/or LCDR3 has 1, 2, or 3 mutations.

In some embodiments, the TCRβV12-3-binding domain comprises: a Kabat HCDR1 that comprises the amino acid sequence of ITGMH (SEQ ID NO: 1168); a Kabat HCDR2 that comprises the amino acid sequence of YISSWSSTIYYADTLKG (SEQ ID NO: 1177); a Kabat HCDR3 that comprises the amino acid sequence of RGAGAMDY (SEQ ID NO: 1181); a Kabat LCDR1 that comprises the amino acid sequence of RASKSVNYIY (SEQ ID NO: 1153); a Kabat LCDR2 that comprises the amino acid sequence of YTSNLAP (SEQ ID NO: 1156); and a Kabat LCDR3 that comprises the amino acid sequence of QQFTSSPFT (SEQ ID NO: 1157). In some embodiments, the HCDR1, HCDR2, HCDR3, LCDR1, LCDR2, and/or LCDR3 has 1, 2, or 3 mutations.

In some embodiments, the TCRβV12-3-binding domain comprises: a Kabat HCDR1 that comprises the amino acid sequence of IKGMH (SEQ ID NO: 1164); a Kabat HCDR2 that comprises the amino acid sequence of YISSSSSTILYADTLKG (SEQ ID NO: 1174); a Kabat HCDR3 that comprises the amino acid sequence of RGAGAMDY (SEQ ID NO: 1181); a Kabat LCDR1 that comprises the amino acid sequence of RASKSVNYIY (SEQ ID NO: 1153); a Kabat LCDR2 that comprises the amino acid sequence of YTSNLAP (SEQ ID NO: 1156); and a Kabat LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises: a Kabat HCDR1 that comprises the amino acid sequence of RKGMH (SEQ ID NO: 1167); a Kabat HCDR2 that comprises the amino acid sequence of YISSWSSTIYYADTLKG (SEQ ID NO: 1177); a Kabat HCDR3 that comprises the amino acid sequence of RGAGAMDY (SEQ ID NO: 1181); a Kabat LCDR1 that comprises the amino acid sequence of RASSSVNYAY (SEQ ID NO: 1154); a Kabat LCDR2 that comprises the amino acid sequence of YTSNLAP (SEQ ID NO: 1156); and a Kabat LCDR3 that comprises the amino acid sequence of QQFTSSPFT (SEQ ID NO: 1157). In some embodiments, the HCDR1, HCDR2, HCDR3, LCDR1, LCDR2, and/or LCDR3 has 1, 2, or 3 mutations.

In some embodiments, the TCRβV12-3-binding domain comprises: a Kabat HCDR1 that comprises the amino acid sequence of IFGMH (SEQ ID NO: 1162); a Kabat HCDR2 that comprises the amino acid sequence of YISSTSSTILYADTLKG (SEQ ID NO: 1173); a Kabat HCDR3 that comprises the amino acid sequence of RGEGAMDY (SEQ ID NO: 1180); a Kabat LCDR1 that comprises the amino acid sequence of RASRSVNYAY (SEQ ID NO: 1151); a Kabat LCDR2 that comprises the amino acid sequence of YTSNLAP (SEQ ID NO: 1156); and a Kabat LCDR3 that comprises the amino acid sequence of QQFTSSPFT (SEQ ID NO: 1157). In some embodiments, the HCDR1, HCDR2, HCDR3, LCDR1, LCDR2, and/or LCDR3 has 1, 2, or 3 mutations.

In some embodiments, the TCRβV12-3-binding domain comprises: a Kabat HCDR1 that comprises the amino acid sequence of IYGMH (SEQ ID NO: 1169); a Kabat HCDR2 that comprises the amino acid sequence of YISSTSSTILYADTLKG (SEQ ID NO: 1173); a Kabat HCDR3 that comprises the amino acid sequence of RGAGAMDY (SEQ ID NO: 1181); a Kabat LCDR1 that comprises the amino acid sequence of RASSSVNYAY (SEQ ID NO: 1154); a Kabat LCDR2 that comprises the amino acid sequence of YTSNLAP (SEQ ID NO: 1156); and a Kabat LCDR3 that comprises the amino acid sequence of QQFTSSPFT (SEQ ID NO: 1157). In some embodiments, the HCDR1, HCDR2, HCDR3, LCDR1, LCDR2, and/or LCDR3 has 1, 2, or 3 mutations.

In some embodiments, the TCRβV12-3-binding domain comprises: a Kabat HCDR1 that comprises the amino acid sequence of RTGMH (SEQ ID NO: 1163); a Kabat HCDR2 that comprises the amino acid sequence of YISSGSSTILYADTLKG (SEQ ID NO: 1179); a Kabat HCDR3 that comprises the amino acid sequence of RGEGAMDY (SEQ ID NO: 1180); a Kabat LCDR1 that comprises the amino acid sequence of RASKSVNYAY (SEQ ID NO: 1152); a Kabat LCDR2 that comprises the amino acid sequence of YTSNLAP (SEQ ID NO: 1156); and a Kabat LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises: a Kabat HCDR1 that comprises the amino acid sequence of NTGMH (SEQ ID NO: 1170); a Kabat HCDR2 that comprises the amino acid sequence of YISSTSSTILYADTLKG (SEQ ID NO: 1173); a Kabat HCDR3 that comprises the amino acid sequence of RGAGAMDY (SEQ ID NO: 1181); a Kabat LCDR1 that comprises the amino acid sequence of RASRSVNYAY (SEQ ID NO: 1151); a Kabat LCDR2 that comprises the amino acid sequence of YTSNLAP (SEQ ID NO: 1156); and a Kabat LCDR3 that comprises the amino acid sequence of QQFTSSPFT (SEQ ID NO: 1157). In some embodiments, the HCDR1, HCDR2, HCDR3, LCDR1, LCDR2, and/or LCDR3 has 1, 2, or 3 mutations.

In some embodiments, the TCRβV12-3-binding domain comprises: a Kabat HCDR1 that comprises the amino acid sequence of NKGMH (SEQ ID NO: 1165); a Kabat HCDR2 that comprises the amino acid sequence of YISSGSSTILYADTLKG (SEQ ID NO: 1179); a Kabat HCDR3 that comprises the amino acid sequence of RGEGAMDY (SEQ ID NO: 1180); a Kabat LCDR1 that comprises the amino acid sequence of RASKSVNYAY (SEQ ID NO: 1152); a Kabat LCDR2 that comprises the amino acid sequence of YTSNLAP (SEQ ID NO: 1156); and a Kabat LCDR3 that comprises the amino acid sequence of QQFTSSPFT (SEQ ID NO: 1157). In some embodiments, the HCDR1, HCDR2, HCDR3, LCDR1, LCDR2, and/or LCDR3 has 1, 2, or 3 mutations.

In some embodiments, the TCRβV12-3-binding domain comprises: a Kabat HCDR1 that comprises the amino acid sequence of ITGMH (SEQ ID NO: 1168); a Kabat HCDR2 that comprises the amino acid sequence of YISSTSSTILYADTLKG (SEQ ID NO: 1173); a Kabat HCDR3 that comprises the amino acid sequence of RGAGAMDY (SEQ ID NO: 1181); a Kabat LCDR1 that comprises the amino acid sequence of RASRSVNYIY (SEQ ID NO: 1155); a Kabat LCDR2 that comprises the amino acid sequence of YTSNLAP (SEQ ID NO: 1156); and a Kabat LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises: a Kabat HCDR1 that comprises the amino acid sequence of RFGMH (SEQ ID NO: 1166); a Kabat HCDR2 that comprises the amino acid sequence of YISSTSSTIYYADTLKG (SEQ ID NO: 1178); a Kabat HCDR3 that comprises the amino acid sequence of RGEGAMDY (SEQ ID NO: 1180); a Kabat LCDR1 that comprises the amino acid sequence of RASRSVNYAY (SEQ ID NO: 1151); a Kabat LCDR2 that comprises the amino acid sequence of YTSNLAP (SEQ ID NO: 1156); and a Kabat LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises: a Kabat HCDR1 that comprises the amino acid sequence of ITGMH (SEQ ID NO: 1168); a Kabat HCDR2 that comprises the amino acid sequence of YISSSSSTIYYADTLKG (SEQ ID NO: 1175); a Kabat HCDR3 that comprises the amino acid sequence of RGAGAMDY (SEQ ID NO: 1181); a Kabat LCDR1 that comprises the amino acid sequence of RASKSVNYAY (SEQ ID NO: 1152); a Kabat LCDR2 that comprises the amino acid sequence of YTSNLAP (SEQ ID NO: 1156); and a Kabat LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises: a Kabat HCDR1 that comprises the amino acid sequence of RTGMH (SEQ ID NO: 1163); a Kabat HCDR2 that comprises the amino acid sequence of YISSWSSTIYYADTLKG (SEQ ID NO: 1177); a Kabat HCDR3 that comprises the amino acid sequence of RGEGAMDY (SEQ ID NO: 1180); a Kabat LCDR1 that comprises the amino acid sequence of RASSSVNYAY (SEQ ID NO: 1154); a Kabat LCDR2 that comprises the amino acid sequence of YTSNLAP (SEQ ID NO: 1156); and a Kabat LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises: a Kabat HCDR1 that comprises the amino acid sequence of RKGMH (SEQ ID NO: 1167); a Kabat HCDR2 that comprises the amino acid sequence of YISSWSSTIYYADTLKG (SEQ ID NO: 1177); a Kabat HCDR3 that comprises the amino acid sequence of RGAGAMDY (SEQ ID NO: 1181); a Kabat LCDR1 that comprises the amino acid sequence of RASKSVNYIY (SEQ ID NO: 1153); a Kabat LCDR2 that comprises the amino acid sequence of YTSNLAP (SEQ ID NO: 1156); and a Kabat LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises: a Kabat HCDR1 that comprises the amino acid sequence of NFGMH (SEQ ID NO: 1160); a Kabat HCDR2 that comprises the amino acid sequence of YISSWSSTIYYADTLKG (SEQ ID NO: 1177); a Kabat HCDR3 that comprises the amino acid sequence of RGAGAMDY (SEQ ID NO: 1181); a Kabat LCDR1 that comprises the amino acid sequence of RASKSVNYIY (SEQ ID NO: 1153); a Kabat LCDR2 that comprises the amino acid sequence of YTSNLAP (SEQ ID NO: 1156); and a Kabat LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises: a Kabat HCDR1 that comprises the amino acid sequence of NTGMH (SEQ ID NO: 1170); a Kabat HCDR2 that comprises the amino acid sequence of YISSTSSTILYADTLKG (SEQ ID NO: 1173); a Kabat HCDR3 that comprises the amino acid sequence of RGEGAMDY (SEQ ID NO: 1180); a Kabat LCDR1 that comprises the amino acid sequence of RASRSVNYAY (SEQ ID NO: 1151); a Kabat LCDR2 that comprises the amino acid sequence of YTSNLAP (SEQ ID NO: 1156); and a Kabat LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises: a Kabat HCDR1 that comprises the amino acid sequence of NTGMH (SEQ ID NO: 1170); a Kabat HCDR2 that comprises the amino acid sequence of YISSSSSTILYADTLKG (SEQ ID NO: 1174); a Kabat HCDR3 that comprises the amino acid sequence of RGAGAMDY (SEQ ID NO: 1181); a Kabat LCDR1 that comprises the amino acid sequence of RASRSVNYAY (SEQ ID NO: 1151); a Kabat LCDR2 that comprises the amino acid sequence of YTSNLAP (SEQ ID NO: 1156); and a Kabat LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises: a Kabat HCDR1 that comprises the amino acid sequence of RTGMH (SEQ ID NO: 1163); a Kabat HCDR2 that comprises the amino acid sequence of YISSWSSTILYADTLKG (SEQ ID NO: 1176); a Kabat HCDR3 that comprises the amino acid sequence of RGAGAMDY (SEQ ID NO: 1181); a Kabat LCDR1 that comprises the amino acid sequence of RASRSVNYAY (SEQ ID NO: 1151); a Kabat LCDR2 that comprises the amino acid sequence of YTSNLAP (SEQ ID NO: 1156); and a Kabat LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises: a Kabat HCDR1 that comprises the amino acid sequence of NKGMH (SEQ ID NO: 1165); a Kabat HCDR2 that comprises the amino acid sequence of YISSWSSTILYADTLKG (SEQ ID NO: 1176); a Kabat HCDR3 that comprises the amino acid sequence of RGAGAMDY (SEQ ID NO: 1181); a Kabat LCDR1 that comprises the amino acid sequence of RASSSVNYAY (SEQ ID NO: 1154); a Kabat LCDR2 that comprises the amino acid sequence of YTSNLAP (SEQ ID NO: 1156); and a Kabat LCDR3 that comprises the amino acid sequence of QQFTSSPFT (SEQ ID NO: 1157). In some embodiments, the HCDR1, HCDR2, HCDR3, LCDR1, LCDR2, and/or LCDR3 has 1, 2, or 3 mutations.

In some embodiments, the TCRβV12-3-binding domain comprises: a Kabat HCDR1 that comprises the amino acid sequence of NKGMH (SEQ ID NO: 1165); a Kabat HCDR2 that comprises the amino acid sequence of YISSGSSTILYADTLKG (SEQ ID NO: 1179); a Kabat HCDR3 that comprises the amino acid sequence of RGAGAMDY (SEQ ID NO: 1181); a Kabat LCDR1 that comprises the amino acid sequence of RASRSVNYAY (SEQ ID NO: 1151); a Kabat LCDR2 that comprises the amino acid sequence of YTSNLAP (SEQ ID NO: 1156); and a Kabat LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises: a Kabat HCDR1 that comprises the amino acid sequence of RTGMH (SEQ ID NO: 1163); a Kabat HCDR2 that comprises the amino acid sequence of YISSSSSTILYADTLKG (SEQ ID NO: 1174); a Kabat HCDR3 that comprises the amino acid sequence of RGEGAMDY (SEQ ID NO: 1180); a Kabat LCDR1 that comprises the amino acid sequence of RASKSVNYAY (SEQ ID NO: 1152); a Kabat LCDR2 that comprises the amino acid sequence of YTSNLAP (SEQ ID NO: 1156); and a Kabat LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises: a Kabat HCDR1 that comprises the amino acid sequence of NTGMH (SEQ ID NO: 1170); a Kabat HCDR2 that comprises the amino acid sequence of YISSSSSTIYYADTLKG (SEQ ID NO: 1175); a Kabat HCDR3 that comprises the amino acid sequence of RGAGAMDY (SEQ ID NO: 1181); a Kabat LCDR1 that comprises the amino acid sequence of RASRSVNYAY (SEQ ID NO: 1151); a Kabat LCDR2 that comprises the amino acid sequence of YTSNLAP (SEQ ID NO: 1156); and a Kabat LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises: a Kabat HCDR1 that comprises the amino acid sequence of ITGMH (SEQ ID NO: 1168); a Kabat HCDR2 that comprises the amino acid sequence of YISSSSSTILYADTLKG (SEQ ID NO: 1174); a Kabat HCDR3 that comprises the amino acid sequence of RGEGAMDY (SEQ ID NO: 1180); a Kabat LCDR1 that comprises the amino acid sequence of RASRSVNYIY (SEQ ID NO: 1155); a Kabat LCDR2 that comprises the amino acid sequence of YTSNLAP (SEQ ID NO: 1156); and a Kabat LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises: an IMGT HCDR1 that comprises the amino acid sequence of GFTFSIFG (SEQ ID NO: 1188); an IMGT HCDR2 that comprises the amino acid sequence of ISSTSSTI (SEQ ID NO: 1198); an IMGT HCDR3 that comprises the amino acid sequence of ARRGEGAMDY (SEQ ID NO: 1201); an IMGT LCDR1 that comprises the amino acid sequence of RSVNY (SEQ ID NO: 1184); an IMGT LCDR2 that comprises the amino acid sequence of YTS; and an IMGT LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises: an IMGT HCDR1 that comprises the amino acid sequence of GFTFSIFG (SEQ ID NO: 1188); an IMGT HCDR2 that comprises the amino acid sequence of ISSTSSTI (SEQ ID NO: 1198); an IMGT HCDR3 that comprises the amino acid sequence of ARRGEGAMDY (SEQ ID NO: 1201); an IMGT LCDR1 that comprises the amino acid sequence of SSVNY (SEQ ID NO: 1182); an IMGT LCDR2 that comprises the amino acid sequence of YTS; and an IMGT LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises: an IMGT HCDR1 that comprises the amino acid sequence of GFTFSRTG (SEQ ID NO: 1189); an IMGT HCDR2 that comprises the amino acid sequence of ISSSSSTI (SEQ ID NO: 1199); an IMGT HCDR3 that comprises the amino acid sequence of ARRGEGAMDY (SEQ ID NO: 1201); an IMGT LCDR1 that comprises the amino acid sequence of RSVNY (SEQ ID NO: 1184); an IMGT LCDR2 that comprises the amino acid sequence of YTS; and an IMGT LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises: an IMGT HCDR1 that comprises the amino acid sequence of GFTFSIKG (SEQ ID NO: 1190); an IMGT HCDR2 that comprises the amino acid sequence of ISSGSSTI (SEQ ID NO: 1197); an IMGT HCDR3 that comprises the amino acid sequence of ARRGAGAMDY (SEQ ID NO: 1202); an IMGT LCDR1 that comprises the amino acid sequence of KSVNY (SEQ ID NO: 1185); an IMGT LCDR2 that comprises the amino acid sequence of YTS; and an IMGT LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises: an IMGT HCDR1 that comprises the amino acid sequence of GFTFSNKG (SEQ ID NO: 1191); an IMGT HCDR2 that comprises the amino acid sequence of ISSSSSTI (SEQ ID NO: 1199); an IMGT HCDR3 that comprises the amino acid sequence of ARRGEGAMDY (SEQ ID NO: 1201); an IMGT LCDR1 that comprises the amino acid sequence of KSVNY (SEQ ID NO: 1185); an IMGT LCDR2 that comprises the amino acid sequence of YTS; and an IMGT LCDR3 that comprises the amino acid sequence of QQFTSSPFT (SEQ ID NO: 1157). In some embodiments, the HCDR1, HCDR2, HCDR3, LCDR1, LCDR2, and/or LCDR3 has 1, 2, or 3 mutations.

In some embodiments, the TCRβV12-3-binding domain comprises: an IMGT HCDR1 that comprises the amino acid sequence of GFTFSNFG (SEQ ID NO: 1186); an IMGT HCDR2 that comprises the amino acid sequence of ISSWSSTI (SEQ ID NO: 1200); an IMGT HCDR3 that comprises the amino acid sequence of ARRGAGAMDY (SEQ ID NO: 1202); an IMGT LCDR1 that comprises the amino acid sequence of SSVNY (SEQ ID NO: 1182); an IMGT LCDR2 that comprises the amino acid sequence of YTS; and an IMGT LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises: an IMGT HCDR1 that comprises the amino acid sequence of GFTFSIFG (SEQ ID NO: 1188); an IMGT HCDR2 that comprises the amino acid sequence of ISSWSSTI (SEQ ID NO: 1200); an IMGT HCDR3 that comprises the amino acid sequence of ARRGEGAMDY (SEQ ID NO: 1201); an IMGT LCDR1 that comprises the amino acid sequence of RSVNY (SEQ ID NO: 1184); an IMGT LCDR2 that comprises the amino acid sequence of YTS; and an IMGT LCDR3 that comprises the amino acid sequence of QQFTSSPFT (SEQ ID NO: 1157). In some embodiments, the HCDR1, HCDR2, HCDR3, LCDR1, LCDR2, and/or LCDR3 has 1, 2, or 3 mutations.

In some embodiments, the TCRβV12-3-binding domain comprises: an IMGT HCDR1 that comprises the amino acid sequence of GFTFSRFG (SEQ ID NO: 1192); an IMGT HCDR2 that comprises the amino acid sequence of ISSWSSTI (SEQ ID NO: 1200); an IMGT HCDR3 that comprises the amino acid sequence of ARRGEGAMDY (SEQ ID NO: 1201); an IMGT LCDR1 that comprises the amino acid sequence of KSVNY (SEQ ID NO: 1185); an IMGT LCDR2 that comprises the amino acid sequence of YTS; and an IMGT LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises: an IMGT HCDR1 that comprises the amino acid sequence of GFTFSRKG (SEQ ID NO: 1193); an IMGT HCDR2 that comprises the amino acid sequence of ISSTSSTI (SEQ ID NO: 1198); an IMGT HCDR3 that comprises the amino acid sequence of ARRGEGAMDY (SEQ ID NO: 1201); an IMGT LCDR1 that comprises the amino acid sequence of RSVNY (SEQ ID NO: 1184); an IMGT LCDR2 that comprises the amino acid sequence of YTS; and an IMGT LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises: an IMGT HCDR1 that comprises the amino acid sequence of GFTFSIFG (SEQ ID NO: 1188); an IMGT HCDR2 that comprises the amino acid sequence of ISSWSSTI (SEQ ID NO: 1200); an IMGT HCDR3 that comprises the amino acid sequence of ARRGAGAMDY (SEQ ID NO: 1202); an IMGT LCDR1 that comprises the amino acid sequence of SSVNY (SEQ ID NO: 1182); an IMGT LCDR2 that comprises the amino acid sequence of YTS; and an IMGT LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises: an IMGT HCDR1 that comprises the amino acid sequence of GFTFSIFG (SEQ ID NO: 1188); an IMGT HCDR2 that comprises the amino acid sequence of ISSSSSTI (SEQ ID NO: 1199); an IMGT HCDR3 that comprises the amino acid sequence of ARRGAGAMDY (SEQ ID NO: 1202); an IMGT LCDR1 that comprises the amino acid sequence of SSVNY (SEQ ID NO: 1182); an IMGT LCDR2 that comprises the amino acid sequence of YTS; and an IMGT LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises: an IMGT HCDR1 that comprises the amino acid sequence of GFTFSNKG (SEQ ID NO: 1191); an IMGT HCDR2 that comprises the amino acid sequence of ISSSSSTI (SEQ ID NO: 1199); an IMGT HCDR3 that comprises the amino acid sequence of ARRGAGAMDY (SEQ ID NO: 1202); an IMGT LCDR1 that comprises the amino acid sequence of SSVNY (SEQ ID NO: 1182); an IMGT LCDR2 that comprises the amino acid sequence of YTS; and an IMGT LCDR3 that comprises the amino acid sequence of QQFTSSPFT (SEQ ID NO: 1157). In some embodiments, the HCDR1, HCDR2, HCDR3, LCDR1, LCDR2, and/or LCDR3 has 1, 2, or 3 mutations.

In some embodiments, the TCRβV12-3-binding domain comprises: an IMGT HCDR1 that comprises the amino acid sequence of GFTFSRFG (SEQ ID NO: 1192); an IMGT HCDR2 that comprises the amino acid sequence of ISSTSSTI (SEQ ID NO: 1198); an IMGT HCDR3 that comprises the amino acid sequence of ARRGEGAMDY (SEQ ID NO: 1201); an IMGT LCDR1 that comprises the amino acid sequence of RSVNY (SEQ ID NO: 1184); an IMGT LCDR2 that comprises the amino acid sequence of YTS; and an IMGT LCDR3 that comprises the amino acid sequence of QQFTSSPFT (SEQ ID NO: 1157). In some embodiments, the HCDR1, HCDR2, HCDR3, LCDR1, LCDR2, and/or LCDR3 has 1, 2, or 3 mutations.

In some embodiments, the TCRβV12-3-binding domain comprises: an IMGT HCDR1 that comprises the amino acid sequence of GFTFSITG (SEQ ID NO: 1194); an IMGT HCDR2 that comprises the amino acid sequence of ISSTSSTI (SEQ ID NO: 1198); an IMGT HCDR3 that comprises the amino acid sequence of ARRGAGAMDY (SEQ ID NO: 1202); an IMGT LCDR1 that comprises the amino acid sequence of KSVNY (SEQ ID NO: 1185); an IMGT LCDR2 that comprises the amino acid sequence of YTS; and an IMGT LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises: an IMGT HCDR1 that comprises the amino acid sequence of GFTFSRKG (SEQ ID NO: 1193); an IMGT HCDR2 that comprises the amino acid sequence of ISSGSSTI (SEQ ID NO: 1197); an IMGT HCDR3 that comprises the amino acid sequence of ARRGAGAMDY (SEQ ID NO: 1202); an IMGT LCDR1 that comprises the amino acid sequence of SSVNY (SEQ ID NO: 1182); an IMGT LCDR2 that comprises the amino acid sequence of YTS; and an IMGT LCDR3 that comprises the amino acid sequence of QQFTSSPFT (SEQ ID NO: 1157). In some embodiments, the HCDR1, HCDR2, HCDR3, LCDR1, LCDR2, and/or LCDR3 has 1, 2, or 3 mutations.

In some embodiments, the TCRβV12-3-binding domain comprises: an IMGT HCDR1 that comprises the amino acid sequence of GFTFSITG (SEQ ID NO: 1194); an IMGT HCDR2 that comprises the amino acid sequence of ISSWSSTI (SEQ ID NO: 1200); an IMGT HCDR3 that comprises the amino acid sequence of ARRGAGAMDY (SEQ ID NO: 1202); an IMGT LCDR1 that comprises the amino acid sequence of KSVNY (SEQ ID NO: 1185); an IMGT LCDR2 that comprises the amino acid sequence of YTS; and an IMGT LCDR3 that comprises the amino acid sequence of QQFTSSPFT (SEQ ID NO: 1157). In some embodiments, the HCDR1, HCDR2, HCDR3, LCDR1, LCDR2, and/or LCDR3 has 1, 2, or 3 mutations.

In some embodiments, the TCRβV12-3-binding domain comprises: an IMGT HCDR1 that comprises the amino acid sequence of GFTFSIKG (SEQ ID NO: 1190); an IMGT HCDR2 that comprises the amino acid sequence of ISSSSSTI (SEQ ID NO: 1199); an IMGT HCDR3 that comprises the amino acid sequence of ARRGAGAMDY (SEQ ID NO: 1202); an IMGT LCDR1 that comprises the amino acid sequence of KSVNY (SEQ ID NO: 1185); an IMGT LCDR2 that comprises the amino acid sequence of YTS; and an IMGT LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises: an IMGT HCDR1 that comprises the amino acid sequence of GFTFSRKG (SEQ ID NO: 1193); an IMGT HCDR2 that comprises the amino acid sequence of ISSWSSTI (SEQ ID NO: 1200); an IMGT HCDR3 that comprises the amino acid sequence of ARRGAGAMDY (SEQ ID NO: 1202); an IMGT LCDR1 that comprises the amino acid sequence of SSVNY (SEQ ID NO: 1182); an IMGT LCDR2 that comprises the amino acid sequence of YTS; and an IMGT LCDR3 that comprises the amino acid sequence of QQFTSSPFT (SEQ ID NO: 1157). In some embodiments, the HCDR1, HCDR2, HCDR3, LCDR1, LCDR2, and/or LCDR3 has 1, 2, or 3 mutations.

In some embodiments, the TCRβV12-3-binding domain comprises: an IMGT HCDR1 that comprises the amino acid sequence of GFTFSIFG (SEQ ID NO: 1188); an IMGT HCDR2 that comprises the amino acid sequence of ISSTSSTI (SEQ ID NO: 1198); an IMGT HCDR3 that comprises the amino acid sequence of ARRGEGAMDY (SEQ ID NO: 1201); an IMGT LCDR1 that comprises the amino acid sequence of RSVNY (SEQ ID NO: 1184); an IMGT LCDR2 that comprises the amino acid sequence of YTS; and an IMGT LCDR3 that comprises the amino acid sequence of QQFTSSPFT (SEQ ID NO: 1157). In some embodiments, the HCDR1, HCDR2, HCDR3, LCDR1, LCDR2, and/or LCDR3 has 1, 2, or 3 mutations.

In some embodiments, the TCRβV12-3-binding domain comprises: an IMGT HCDR1 that comprises the amino acid sequence of GFTFSIYG (SEQ ID NO: 1195); an IMGT HCDR2 that comprises the amino acid sequence of ISSTSSTI (SEQ ID NO: 1198); an IMGT HCDR3 that comprises the amino acid sequence of ARRGAGAMDY (SEQ ID NO: 1202); an IMGT LCDR1 that comprises the amino acid sequence of SSVNY (SEQ ID NO: 1182); an IMGT LCDR2 that comprises the amino acid sequence of YTS; and an IMGT LCDR3 that comprises the amino acid sequence of QQFTSSPFT (SEQ ID NO: 1157). In some embodiments, the HCDR1, HCDR2, HCDR3, LCDR1, LCDR2, and/or LCDR3 has 1, 2, or 3 mutations.

In some embodiments, the TCRβV12-3-binding domain comprises: an IMGT HCDR1 that comprises the amino acid sequence of GFTFSRTG (SEQ ID NO: 1189); an IMGT HCDR2 that comprises the amino acid sequence of ISSGSSTI (SEQ ID NO: 1197); an IMGT HCDR3 that comprises the amino acid sequence of ARRGEGAMDY (SEQ ID NO: 1201); an IMGT LCDR1 that comprises the amino acid sequence of KSVNY (SEQ ID NO: 1185); an IMGT LCDR2 that comprises the amino acid sequence of YTS; and an IMGT LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises: an IMGT HCDR1 that comprises the amino acid sequence of GFTFSNTG (SEQ ID NO: 1196); an IMGT HCDR2 that comprises the amino acid sequence of ISSTSSTI (SEQ ID NO: 1198); an IMGT HCDR3 that comprises the amino acid sequence of ARRGAGAMDY (SEQ ID NO: 1202); an IMGT LCDR1 that comprises the amino acid sequence of RSVNY (SEQ ID NO: 1184); an IMGT LCDR2 that comprises the amino acid sequence of YTS; and an IMGT LCDR3 that comprises the amino acid sequence of QQFTSSPFT (SEQ ID NO: 1157). In some embodiments, the HCDR1, HCDR2, HCDR3, LCDR1, LCDR2, and/or LCDR3 has 1, 2, or 3 mutations.

In some embodiments, the TCRβV12-3-binding domain comprises: an IMGT HCDR1 that comprises the amino acid sequence of GFTFSNKG (SEQ ID NO: 1191); an IMGT HCDR2 that comprises the amino acid sequence of ISSGSSTI (SEQ ID NO: 1197); an IMGT HCDR3 that comprises the amino acid sequence of ARRGEGAMDY (SEQ ID NO: 1201); an IMGT LCDR1 that comprises the amino acid sequence of KSVNY (SEQ ID NO: 1185); an IMGT LCDR2 that comprises the amino acid sequence of YTS; and an IMGT LCDR3 that comprises the amino acid sequence of QQFTSSPFT (SEQ ID NO: 1157). In some embodiments, the HCDR1, HCDR2, HCDR3, LCDR1, LCDR2, and/or LCDR3 has 1, 2, or 3 mutations.

In some embodiments, the TCRβV12-3-binding domain comprises: an IMGT HCDR1 that comprises the amino acid sequence of GFTFSITG (SEQ ID NO: 1194); an IMGT HCDR2 that comprises the amino acid sequence of ISSTSSTI (SEQ ID NO: 1198); an IMGT HCDR3 that comprises the amino acid sequence of ARRGAGAMDY (SEQ ID NO: 1202); an IMGT LCDR1 that comprises the amino acid sequence of RSVNY (SEQ ID NO: 1184); an IMGT LCDR2 that comprises the amino acid sequence of YTS; and an IMGT LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises: an IMGT HCDR1 that comprises the amino acid sequence of GFTFSRFG (SEQ ID NO: 1192); an IMGT HCDR2 that comprises the amino acid sequence of ISSTSSTI (SEQ ID NO: 1198); an IMGT HCDR3 that comprises the amino acid sequence of ARRGEGAMDY (SEQ ID NO: 1201); an IMGT LCDR1 that comprises the amino acid sequence of RSVNY (SEQ ID NO: 1184); an IMGT LCDR2 that comprises the amino acid sequence of YTS; and an IMGT LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises: an IMGT HCDR1 that comprises the amino acid sequence of GFTFSITG (SEQ ID NO: 1194); an IMGT HCDR2 that comprises the amino acid sequence of ISSSSSTI (SEQ ID NO: 1199); an IMGT HCDR3 that comprises the amino acid sequence of ARRGAGAMDY (SEQ ID NO: 1202); an IMGT LCDR1 that comprises the amino acid sequence of KSVNY (SEQ ID NO: 1185); an IMGT LCDR2 that comprises the amino acid sequence of YTS; and an IMGT LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises: an IMGT HCDR1 that comprises the amino acid sequence of GFTFSRTG (SEQ ID NO: 1189); an IMGT HCDR2 that comprises the amino acid sequence of ISSWSSTI (SEQ ID NO: 1200); an IMGT HCDR3 that comprises the amino acid sequence of ARRGEGAMDY (SEQ ID NO: 1201); an IMGT LCDR1 that comprises the amino acid sequence of SSVNY (SEQ ID NO: 1182); an IMGT LCDR2 that comprises the amino acid sequence of YTS; and an IMGT LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises: an IMGT HCDR1 that comprises the amino acid sequence of GFTFSRKG (SEQ ID NO: 1193); an IMGT HCDR2 that comprises the amino acid sequence of ISSWSSTI (SEQ ID NO: 1200); an IMGT HCDR3 that comprises the amino acid sequence of ARRGAGAMDY (SEQ ID NO: 1202); an IMGT LCDR1 that comprises the amino acid sequence of KSVNY (SEQ ID NO: 1185); an IMGT LCDR2 that comprises the amino acid sequence of YTS; and an IMGT LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises: an IMGT HCDR1 that comprises the amino acid sequence of GFTFSNFG (SEQ ID NO: 1186); an IMGT HCDR2 that comprises the amino acid sequence of ISSWSSTI (SEQ ID NO: 1200); an IMGT HCDR3 that comprises the amino acid sequence of ARRGAGAMDY (SEQ ID NO: 1202); an IMGT LCDR1 that comprises the amino acid sequence of KSVNY (SEQ ID NO: 1185); an IMGT LCDR2 that comprises the amino acid sequence of YTS; and an IMGT LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises: an IMGT HCDR1 that comprises the amino acid sequence of GFTFSNTG (SEQ ID NO: 1196); an IMGT HCDR2 that comprises the amino acid sequence of ISSTSSTI (SEQ ID NO: 1198); an IMGT HCDR3 that comprises the amino acid sequence of ARRGEGAMDY (SEQ ID NO: 1201); an IMGT LCDR1 that comprises the amino acid sequence of RSVNY (SEQ ID NO: 1184); an IMGT LCDR2 that comprises the amino acid sequence of YTS; and an IMGT LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises: an IMGT HCDR1 that comprises the amino acid sequence of GFTFSNTG (SEQ ID NO: 1196); an IMGT HCDR2 that comprises the amino acid sequence of ISSSSSTI (SEQ ID NO: 1199); an IMGT HCDR3 that comprises the amino acid sequence of ARRGAGAMDY (SEQ ID NO: 1202); an IMGT LCDR1 that comprises the amino acid sequence of RSVNY (SEQ ID NO: 1184); an IMGT LCDR2 that comprises the amino acid sequence of YTS; and an IMGT LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises: an IMGT HCDR1 that comprises the amino acid sequence of GFTFSRTG (SEQ ID NO: 1189); an IMGT HCDR2 that comprises the amino acid sequence of ISSWSSTI (SEQ ID NO: 1200); an IMGT HCDR3 that comprises the amino acid sequence of ARRGAGAMDY (SEQ ID NO: 1202); an IMGT LCDR1 that comprises the amino acid sequence of RSVNY (SEQ ID NO: 1184); an IMGT LCDR2 that comprises the amino acid sequence of YTS; and an IMGT LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises: an IMGT HCDR1 that comprises the amino acid sequence of GFTFSNKG (SEQ ID NO: 1191); an IMGT HCDR2 that comprises the amino acid sequence of ISSWSSTI (SEQ ID NO: 1200); an IMGT HCDR3 that comprises the amino acid sequence of ARRGAGAMDY (SEQ ID NO: 1202); an IMGT LCDR1 that comprises the amino acid sequence of SSVNY (SEQ ID NO: 1182); an IMGT LCDR2 that comprises the amino acid sequence of YTS; and an IMGT LCDR3 that comprises the amino acid sequence of QQFTSSPFT (SEQ ID NO: 1157). In some embodiments, the HCDR1, HCDR2, HCDR3, LCDR1, LCDR2, and/or LCDR3 has 1, 2, or 3 mutations.

In some embodiments, the TCRβV12-3-binding domain comprises: an IMGT HCDR1 that comprises the amino acid sequence of GFTFSNKG (SEQ ID NO: 1191); an IMGT HCDR2 that comprises the amino acid sequence of ISSGSSTI (SEQ ID NO: 1197); an IMGT HCDR3 that comprises the amino acid sequence of ARRGAGAMDY (SEQ ID NO: 1202); an IMGT LCDR1 that comprises the amino acid sequence of RSVNY (SEQ ID NO: 1184); an IMGT LCDR2 that comprises the amino acid sequence of YTS; and an IMGT LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises: an IMGT HCDR1 that comprises the amino acid sequence of GFTFSRTG (SEQ ID NO: 1189); an IMGT HCDR2 that comprises the amino acid sequence of ISSSSSTI (SEQ ID NO: 1199); an IMGT HCDR3 that comprises the amino acid sequence of ARRGEGAMDY (SEQ ID NO: 1201); an IMGT LCDR1 that comprises the amino acid sequence of KSVNY (SEQ ID NO: 1185); an IMGT LCDR2 that comprises the amino acid sequence of YTS; and an IMGT LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises: an IMGT HCDR1 that comprises the amino acid sequence of GFTFSNTG (SEQ ID NO: 1196); an IMGT HCDR2 that comprises the amino acid sequence of ISSSSSTI (SEQ ID NO: 1199); an IMGT HCDR3 that comprises the amino acid sequence of ARRGAGAMDY (SEQ ID NO: 1202); an IMGT LCDR1 that comprises the amino acid sequence of RSVNY (SEQ ID NO: 1184); an IMGT LCDR2 that comprises the amino acid sequence of YTS; and an IMGT LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises: an IMGT HCDR1 that comprises the amino acid sequence of GFTFSITG (SEQ ID NO: 1194); an IMGT HCDR2 that comprises the amino acid sequence of ISSSSSTI (SEQ ID NO: 1199); an IMGT HCDR3 that comprises the amino acid sequence of ARRGEGAMDY (SEQ ID NO: 1201); an IMGT LCDR1 that comprises the amino acid sequence of RSVNY (SEQ ID NO: 1184); an IMGT LCDR2 that comprises the amino acid sequence of YTS; and an IMGT LCDR3 that comprises the amino acid sequence of QQFTSSPFV (SEQ ID NO: 1159). In some embodiments, the HCDR1, HCDR2, HCDR3, LCDR1, LCDR2, and/or LCDR3 has 1, 2, or 3 mutations.

In some embodiments, the TCRβV12-3-binding domain comprises a VH comprising an amino acid sequence with at least about 85%, 90%, 95%, 96%, 97%, 98%, 99%, or 100% identity to SEQ ID NO: 1116; and a VL comprising an amino acid sequence with at least about 85%, 90%, 95%, 96%, 97%, 98%, 99%, or 100% identity to SEQ ID NO: 1104. In some embodiments, the TCRβV12-3-binding domain comprises a VH comprising an amino acid sequence of SEQ ID NO: 1116; and a VL comprising an amino acid sequence of SEQ ID NO: 1104.

In some embodiments, the TCRβV12-3-binding domain comprises a VH comprising an amino acid sequence with at least about 85%, 90%, 95%, 96%, 97%, 98%, 99%, or 100% identity to SEQ ID NO: 1116; and a VL comprising an amino acid sequence with at least about 85%, 90%, 95%, 96%, 97%, 98%, 99%, or 100% identity to SEQ ID NO: 1105. In some embodiments, the TCRβV12-3-binding domain comprises a VH comprising an amino acid sequence of SEQ ID NO: 1116; and a VL comprising an amino acid sequence of SEQ ID NO: 1105.

In some embodiments, the TCRβV12-3-binding domain comprises a VH comprising an amino acid sequence with at least about 85%, 90%, 95%, 96%, 97%, 98%, 99%, or 100% identity to SEQ ID NO: 1117; and a VL comprising an amino acid sequence with at least about 85%, 90%, 95%, 96%, 97%, 98%, 99%, or 100% identity to SEQ ID NO: 1104. In some embodiments, the TCRβV12-3-binding domain comprises a VH comprising an amino acid sequence of SEQ ID NO: 1117; and a VL comprising an amino acid sequence of SEQ ID NO: 1104.

In some embodiments, the TCRβV12-3-binding domain comprises a VH comprising an amino acid sequence with at least about 85%, 90%, 95%, 96%, 97%, 98%, 99%, or 100% identity to SEQ ID NO: 1118; and a VL comprising an amino acid sequence with at least about 85%, 90%, 95%, 96%, 97%, 98%, 99%, or 100% identity to SEQ ID NO: 1106. In some embodiments, the TCRβV12-3-binding domain comprises a VH comprising an amino acid sequence of SEQ ID NO: 1118; and a VL comprising an amino acid sequence of SEQ ID NO: 1106.

In some embodiments, the TCRβV12-3-binding domain comprises a VH comprising an amino acid sequence with at least about 85%, 90%, 95%, 96%, 97%, 98%, 99%, or 100% identity to SEQ ID NO: 1119; and a VL comprising an amino acid sequence with at least about 85%, 90%, 95%, 96%, 97%, 98%, 99%, or 100% identity to SEQ ID NO: 1107. In some embodiments, the TCRβV12-3-binding domain comprises a VH comprising an amino acid sequence of SEQ ID NO: 1119; and a VL comprising an amino acid sequence of SEQ ID NO: 1107.

In some embodiments, the TCRβV12-3-binding domain comprises a VH comprising an amino acid sequence with at least about 85%, 90%, 95%, 96%, 97%, 98%, 99%, or 100% identity to SEQ ID NO: 1120; and a VL comprising an amino acid sequence with at least about 85%, 90%, 95%, 96%, 97%, 98%, 99%, or 100% identity to SEQ ID NO: 1105. In some embodiments, the TCRβV12-3-binding domain comprises a VH comprising an amino acid sequence of SEQ ID NO: 1120; and a VL comprising an amino acid sequence of SEQ ID NO: 1105.

In some embodiments, the TCRβV12-3-binding domain comprises a VH comprising an amino acid sequence with at least about 85%, 90%, 95%, 96%, 97%, 98%, 99%, or 100% identity to SEQ ID NO: 1121; and a VL comprising an amino acid sequence with at least about 85%, 90%, 95%, 96%, 97%, 98%, 99%, or 100% identity to SEQ ID NO: 1108. In some embodiments, the TCRβV12-3-binding domain comprises a VH comprising an amino acid sequence of SEQ ID NO: 1121; and a VL comprising an amino acid sequence of SEQ ID NO: 1108.

In some embodiments, the TCRβV12-3-binding domain comprises a VH comprising an amino acid sequence with at least about 85%, 90%, 95%, 96%, 97%, 98%, 99%, or 100% identity to SEQ ID NO: 1122; and a VL comprising an amino acid sequence with at least about 85%, 90%, 95%, 96%, 97%, 98%, 99%, or 100% identity to SEQ ID NO: 1109. In some embodiments, the TCRβV12-3-binding domain comprises a VH comprising an amino acid sequence of SEQ ID NO: 1122; and a VL comprising an amino acid sequence of SEQ ID NO: 1109.

In some embodiments, the TCRβV12-3-binding domain comprises a VH comprising an amino acid sequence with at least about 85%, 90%, 95%, 96%, 97%, 98%, 99%, or 100% identity to SEQ ID NO: 1123; and a VL comprising an amino acid sequence with at least about 85%, 90%, 95%, 96%, 97%, 98%, 99%, or 100% identity to SEQ ID NO: 1110. In some embodiments, the TCRβV12-3-binding domain comprises a VH comprising an amino acid sequence of SEQ ID NO: 1123; and a VL comprising an amino acid sequence of SEQ ID NO: 1110.

In some embodiments, the TCRβV12-3-binding domain comprises a VH comprising an amino acid sequence with at least about 85%, 90%, 95%, 96%, 97%, 98%, 99%, or 100% identity to SEQ ID NO: 1124; and a VL comprising an amino acid sequence with at least about 85%, 90%, 95%, 96%, 97%, 98%, 99%, or 100% identity to SEQ ID NO: 1105. In some embodiments, the TCRβV12-3-binding domain comprises a VH comprising an amino acid sequence of SEQ ID NO: 1124; and a VL comprising an amino acid sequence of SEQ ID NO: 1105.

In some embodiments, the TCRβV12-3-binding domain comprises a VH comprising an amino acid sequence with at least about 85%, 90%, 95%, 96%, 97%, 98%, 99%, or 100% identity to SEQ ID NO: 1125; and a VL comprising an amino acid sequence with at least about 85%, 90%, 95%, 96%, 97%, 98%, 99%, or 100% identity to SEQ ID NO: 1105. In some embodiments, the TCRβV12-3-binding domain comprises a VH comprising an amino acid sequence of SEQ ID NO: 1125; and a VL comprising an amino acid sequence of SEQ ID NO: 1105.

In some embodiments, the TCRβV12-3-binding domain comprises a VH comprising an amino acid sequence with at least about 85%, 90%, 95%, 96%, 97%, 98%, 99%, or 100% identity to SEQ ID NO: 1126; and a VL comprising an amino acid sequence with at least about 85%, 90%, 95%, 96%, 97%, 98%, 99%, or 100% identity to SEQ ID NO: 1111. In some embodiments, the TCRβV12-3-binding domain comprises a VH comprising an amino acid sequence of SEQ ID NO: 1126; and a VL comprising an amino acid sequence of SEQ ID NO: 1111.

In some embodiments, the TCRβV12-3-binding domain comprises a VH comprising an amino acid sequence with at least about 85%, 90%, 95%, 96%, 97%, 98%, 99%, or 100% identity to SEQ ID NO: 1127; and a VL comprising an amino acid sequence with at least about 85%, 90%, 95%, 96%, 97%, 98%, 99%, or 100% identity to SEQ ID NO: 1108. In some embodiments, the TCRβV12-3-binding domain comprises a VH comprising an amino acid sequence of SEQ ID NO: 1127; and a VL comprising an amino acid sequence of SEQ ID NO: 1108.

In some embodiments, the TCRβV12-3-binding domain comprises a VH comprising an amino acid sequence with at least about 85%, 90%, 95%, 96%, 97%, 98%, 99%, or 100% identity to SEQ ID NO: 1128; and a VL comprising an amino acid sequence with at least about 85%, 90%, 95%, 96%, 97%, 98%, 99%, or 100% identity to SEQ ID NO: 1109. In some embodiments, the TCRβV12-3-binding domain comprises a VH comprising an amino acid sequence of SEQ ID NO: 1128; and a VL comprising an amino acid sequence of SEQ ID NO: 1109.

In some embodiments, the TCRβV12-3-binding domain comprises a VH comprising an amino acid sequence with at least about 85%, 90%, 95%, 96%, 97%, 98%, 99%, or 100% identity to SEQ ID NO: 1129; and a VL comprising an amino acid sequence with at least about 85%, 90%, 95%, 96%, 97%, 98%, 99%, or 100% identity to SEQ ID NO: 1111. In some embodiments, the TCRβV12-3-binding domain comprises a VH comprising an amino acid sequence of SEQ ID NO: 1129; and a VL comprising an amino acid sequence of SEQ ID NO: 1111.

In some embodiments, the TCRβV12-3-binding domain comprises a VH comprising an amino acid sequence with at least about 85%, 90%, 95%, 96%, 97%, 98%, 99%, or 100% identity to SEQ ID NO: 1130; and a VL comprising an amino acid sequence with at least about 85%, 90%, 95%, 96%, 97%, 98%, 99%, or 100% identity to SEQ ID NO: 1107. In some embodiments, the TCRβV12-3-binding domain comprises a VH comprising an amino acid sequence of SEQ ID NO: 1130; and a VL comprising an amino acid sequence of SEQ ID NO: 1107.

In some embodiments, the TCRβV12-3-binding domain comprises a VH comprising an amino acid sequence with at least about 85%, 90%, 95%, 96%, 97%, 98%, 99%, or 100% identity to SEQ ID NO: 1131; and a VL comprising an amino acid sequence with at least about 85%, 90%, 95%, 96%, 97%, 98%, 99%, or 100% identity to SEQ ID NO: 1109. In some embodiments, the TCRβV12-3-binding domain comprises a VH comprising an amino acid sequence of SEQ ID NO: 1131; and a VL comprising an amino acid sequence of SEQ ID NO: 1109.

In some embodiments, the TCRβV12-3-binding domain comprises a VH comprising an amino acid sequence with at least about 85%, 90%, 95%, 96%, 97%, 98%, 99%, or 100% identity to SEQ ID NO: 1132; and a VL comprising an amino acid sequence with at least about 85%, 90%, 95%, 96%, 97%, 98%, 99%, or 100% identity to SEQ ID NO: 1111. In some embodiments, the TCRβV12-3-binding domain comprises a VH comprising an amino acid sequence of SEQ ID NO: 1132; and a VL comprising an amino acid sequence of SEQ ID NO: 1111.

In some embodiments, the TCRβV12-3-binding domain comprises a VH comprising an amino acid sequence with at least about 85%, 90%, 95%, 96%, 97%, 98%, 99%, or 100% identity to SEQ ID NO: 1116; and a VL comprising an amino acid sequence with at least about 85%, 90%, 95%, 96%, 97%, 98%, 99%, or 100% identity to SEQ ID NO: 1108. In some embodiments, the TCRβV12-3-binding domain comprises a VH comprising an amino acid sequence of SEQ ID NO: 1116; and a VL comprising an amino acid sequence of SEQ ID NO: 1108.

In some embodiments, the TCRβV12-3-binding domain comprises a VH comprising an amino acid sequence with at least about 85%, 90%, 95%, 96%, 97%, 98%, 99%, or 100% identity to SEQ ID NO: 1133; and a VL comprising an amino acid sequence with at least about 85%, 90%, 95%, 96%, 97%, 98%, 99%, or 100% identity to SEQ ID NO: 1111. In some embodiments, the TCRβV12-3-binding domain comprises a VH comprising an amino acid sequence of SEQ ID NO: 1133; and a VL comprising an amino acid sequence of SEQ ID NO: 1111.

In some embodiments, the TCRβV12-3-binding domain comprises a VH comprising an amino acid sequence with at least about 85%, 90%, 95%, 96%, 97%, 98%, 99%, or 100% identity to SEQ ID NO: 1134; and a VL comprising an amino acid sequence with at least about 85%, 90%, 95%, 96%, 97%, 98%, 99%, or 100% identity to SEQ ID NO: 1106. In some embodiments, the TCRβV12-3-binding domain comprises a VH comprising an amino acid sequence of SEQ ID NO: 1134; and a VL comprising an amino acid sequence of SEQ ID NO: 1106.

In some embodiments, the TCRβV12-3-binding domain comprises a VH comprising an amino acid sequence with at least about 85%, 90%, 95%, 96%, 97%, 98%, 99%, or 100% identity to SEQ ID NO: 1135; and a VL comprising an amino acid sequence with at least about 85%, 90%, 95%, 96%, 97%, 98%, 99%, or 100% identity to SEQ ID NO: 1108. In some embodiments, the TCRβV12-3-binding domain comprises a VH comprising an amino acid sequence of SEQ ID NO: 1135; and a VL comprising an amino acid sequence of SEQ ID NO: 1108.

In some embodiments, the TCRβV12-3-binding domain comprises a VH comprising an amino acid sequence with at least about 85%, 90%, 95%, 96%, 97%, 98%, 99%, or 100% identity to SEQ ID NO: 1136; and a VL comprising an amino acid sequence with at least about 85%, 90%, 95%, 96%, 97%, 98%, 99%, or 100% identity to SEQ ID NO: 1112. In some embodiments, the TCRβV12-3-binding domain comprises a VH comprising an amino acid sequence of SEQ ID NO: 1136; and a VL comprising an amino acid sequence of SEQ ID NO: 1112.

In some embodiments, the TCRβV12-3-binding domain comprises a VH comprising an amino acid sequence with at least about 85%, 90%, 95%, 96%, 97%, 98%, 99%, or 100% identity to SEQ ID NO: 1137; and a VL comprising an amino acid sequence with at least about 85%, 90%, 95%, 96%, 97%, 98%, 99%, or 100% identity to SEQ ID NO: 1110. In some embodiments, the TCRβV12-3-binding domain comprises a VH comprising an amino acid sequence of SEQ ID NO: 1137; and a VL comprising an amino acid sequence of SEQ ID NO: 1110.

In some embodiments, the TCRβV12-3-binding domain comprises a VH comprising an amino acid sequence with at least about 85%, 90%, 95%, 96%, 97%, 98%, 99%, or 100% identity to SEQ ID NO: 1127; and a VL comprising an amino acid sequence with at least about 85%, 90%, 95%, 96%, 97%, 98%, 99%, or 100% identity to SEQ ID NO: 1104. In some embodiments, the TCRβV12-3-binding domain comprises a VH comprising an amino acid sequence of SEQ ID NO: 1127; and a VL comprising an amino acid sequence of SEQ ID NO: 1104.

In some embodiments, the TCRβV12-3-binding domain comprises a VH comprising an amino acid sequence with at least about 85%, 90%, 95%, 96%, 97%, 98%, 99%, or 100% identity to SEQ ID NO: 1138; and a VL comprising an amino acid sequence with at least about 85%, 90%, 95%, 96%, 97%, 98%, 99%, or 100% identity to SEQ ID NO: 1106. In some embodiments, the TCRβV12-3-binding domain comprises a VH comprising an amino acid sequence of SEQ ID NO: 1138; and a VL comprising an amino acid sequence of SEQ ID NO: 1106.

In some embodiments, the TCRβV12-3-binding domain comprises a VH comprising an amino acid sequence with at least about 85%, 90%, 95%, 96%, 97%, 98%, 99%, or 100% identity to SEQ ID NO: 1139; and a VL comprising an amino acid sequence with at least about 85%, 90%, 95%, 96%, 97%, 98%, 99%, or 100% identity to SEQ ID NO: 1105. In some embodiments, the TCRβV12-3-binding domain comprises a VH comprising an amino acid sequence of SEQ ID NO: 1139; and a VL comprising an amino acid sequence of SEQ ID NO: 1105.

In some embodiments, the TCRβV12-3-binding domain comprises a VH comprising an amino acid sequence with at least about 85%, 90%, 95%, 96%, 97%, 98%, 99%, or 100% identity to SEQ ID NO: 1132; and a VL comprising an amino acid sequence with at least about 85%, 90%, 95%, 96%, 97%, 98%, 99%, or 100% identity to SEQ ID NO: 1109. In some embodiments, the TCRβV12-3-binding domain comprises a VH comprising an amino acid sequence of SEQ ID NO: 1132; and a VL comprising an amino acid sequence of SEQ ID NO: 1109.

In some embodiments, the TCRβV12-3-binding domain comprises a VH comprising an amino acid sequence with at least about 85%, 90%, 95%, 96%, 97%, 98%, 99%, or 100% identity to SEQ ID NO: 1140; and a VL comprising an amino acid sequence with at least about 85%, 90%, 95%, 96%, 97%, 98%, 99%, or 100% identity to SEQ ID NO: 1109. In some embodiments, the TCRβV12-3-binding domain comprises a VH comprising an amino acid sequence of SEQ ID NO: 1140; and a VL comprising an amino acid sequence of SEQ ID NO: 1109.

In some embodiments, the TCRβV12-3-binding domain comprises a VH comprising an amino acid sequence with at least about 85%, 90%, 95%, 96%, 97%, 98%, 99%, or 100% identity to SEQ ID NO: 1141; and a VL comprising an amino acid sequence with at least about 85%, 90%, 95%, 96%, 97%, 98%, 99%, or 100% identity to SEQ ID NO: 1104. In some embodiments, the TCRβV12-3-binding domain comprises a VH comprising an amino acid sequence of SEQ ID NO: 1141; and a VL comprising an amino acid sequence of SEQ ID NO: 1104.

In some embodiments, the TCRβV12-3-binding domain comprises a VH comprising an amino acid sequence with at least about 85%, 90%, 95%, 96%, 97%, 98%, 99%, or 100% identity to SEQ ID NO: 1142; and a VL comprising an amino acid sequence with at least about 85%, 90%, 95%, 96%, 97%, 98%, 99%, or 100% identity to SEQ ID NO: 1104. In some embodiments, the TCRβV12-3-binding domain comprises a VH comprising an amino acid sequence of SEQ ID NO: 1142; and a VL comprising an amino acid sequence of SEQ ID NO: 1104.

In some embodiments, the TCRβV12-3-binding domain comprises a VH comprising an amino acid sequence with at least about 85%, 90%, 95%, 96%, 97%, 98%, 99%, or 100% identity to SEQ ID NO: 1143; and a VL comprising an amino acid sequence with at least about 85%, 90%, 95%, 96%, 97%, 98%, 99%, or 100% identity to SEQ ID NO: 1104. In some embodiments, the TCRβV12-3-binding domain comprises a VH comprising an amino acid sequence of SEQ ID NO: 1143; and a VL comprising an amino acid sequence of SEQ ID NO: 1104.

In some embodiments, the TCRβV12-3-binding domain comprises a VH comprising an amino acid sequence with at least about 85%, 90%, 95%, 96%, 97%, 98%, 99%, or 100% identity to SEQ ID NO: 1144; and a VL comprising an amino acid sequence with at least about 85%, 90%, 95%, 96%, 97%, 98%, 99%, or 100% identity to SEQ ID NO: 1111. In some embodiments, the TCRβV12-3-binding domain comprises a VH comprising an amino acid sequence of SEQ ID NO: 1144; and a VL comprising an amino acid sequence of SEQ ID NO: 1111.

In some embodiments, the TCRβV12-3-binding domain comprises a VH comprising an amino acid sequence with at least about 85%, 90%, 95%, 96%, 97%, 98%, 99%, or 100% identity to SEQ ID NO: 1145; and a VL comprising an amino acid sequence with at least about 85%, 90%, 95%, 96%, 97%, 98%, 99%, or 100% identity to SEQ ID NO: 1104. In some embodiments, the TCRβV12-3-binding domain comprises a VH comprising an amino acid sequence of SEQ ID NO: 1145; and a VL comprising an amino acid sequence of SEQ ID NO: 1104.

In some embodiments, the TCRβV12-3-binding domain comprises a VH comprising an amino acid sequence with at least about 85%, 90%, 95%, 96%, 97%, 98%, 99%, or 100% identity to SEQ ID NO: 1117; and a VL comprising an amino acid sequence with at least about 85%, 90%, 95%, 96%, 97%, 98%, 99%, or 100% identity to SEQ ID NO: 1106. In some embodiments, the TCRβV12-3-binding domain comprises a VH comprising an amino acid sequence of SEQ ID NO: 1117; and a VL comprising an amino acid sequence of SEQ ID NO: 1106.

In some embodiments, the TCRβV12-3-binding domain comprises a VH comprising an amino acid sequence with at least about 85%, 90%, 95%, 96%, 97%, 98%, 99%, or 100% identity to SEQ ID NO: 1146; and a VL comprising an amino acid sequence with at least about 85%, 90%, 95%, 96%, 97%, 98%, 99%, or 100% identity to SEQ ID NO: 1104. In some embodiments, the TCRβV12-3-binding domain comprises a VH comprising an amino acid sequence of SEQ ID NO: 1146; and a VL comprising an amino acid sequence of SEQ ID NO: 1104.

In some embodiments, the TCRβV12-3-binding domain comprises a VH comprising an amino acid sequence with at least about 85%, 90%, 95%, 96%, 97%, 98%, 99%, or 100% identity to SEQ ID NO: 1147; and a VL comprising an amino acid sequence with at least about 85%, 90%, 95%, 96%, 97%, 98%, 99%, or 100% identity to SEQ ID NO: 1110. In some embodiments, the TCRβV12-3-binding domain comprises a VH comprising an amino acid sequence of SEQ ID NO: 1147; and a VL comprising an amino acid sequence of SEQ ID NO: 1110.

In some embodiments, the polypeptide molecule comprises a TCRβV12-3-binding domain. In some embodiments, the TCRβV12-3-binding domain comprises an amino acid sequence of any one of the amino acid sequences in Table 31. In some embodiments, the TCRβV12-3-binding domain comprises an amino acid sequence with at least about or at most about 75%, 80%, 85%, 90%, 91%, 92%, 93%, 94%, 95%, 96%, 97%, 98%, 99%, or 100% identity to any one of the amino acid sequences in Table 31.

In some embodiments, the polypeptide molecule comprises a TCRβV12-3-binding domain. In some embodiments, the TCRβV12-3-binding domain comprises an amino acid sequence of any one of the amino acid sequences in Table 32. In some embodiments, the TCRβV12-3-binding domain comprises an amino acid sequence with at least about or at most about 75%, 80%, 85%, 90%, 91%, 92%, 93%, 94%, 95%, 96%, 97%, 98%, 99%, or 100% identity to any one of the amino acid sequences in Table 32. In some embodiments, the TCRβV12-3-binding domain comprises an amino acid sequence with at least about or at most about 75%, 80%, 85%, 90%, 91%, 92%, 93%, 94%, 95%, 96%, 97%, 98%, 99%, or 100% identical to any one of SEQ ID NOs: 1203-1277. In some embodiments, the TCRβV12-3-binding domain comprises an amino acid sequence of any one of SEQ ID NOs: 1203-1277. In some embodiments, the TCRβV12-3-binding domain consists of an amino acid sequence of any one of SEQ ID NOs: 1203-1277. In some embodiments, the polypeptide molecule is an scFv.

In some embodiments, the TCRβV12-3-binding domain comprises an scFv. In some embodiments, the TCRβV12-3-binding domain comprises an amino acid sequence with at least about or at most about 75%, 80%, 85%, 90%, 91%, 92%, 93%, 94%, 95%, 96%, 97%, 98%, 99%, or 100% identity to SEQ ID NO: 1262. In some embodiments, the TCRβV12-3-binding domain comprises an amino acid sequence of SEQ ID NO: 1262. In some embodiments, the TCRβV12-3-binding domain consists of an amino acid sequence of SEQ ID NO: 1262.

In some embodiments, the TCRβV12-3-binding domain comprises an scFv. In some embodiments, the TCRβV12-3-binding domain comprises an amino acid sequence with at least about or at most about 75%, 80%, 85%, 90%, 91%, 92%, 93%, 94%, 95%, 96%, 97%, 98%, 99%, or 100% identity to SEQ ID NO: 1264. In some embodiments, the TCRβV12-3-binding domain comprises an amino acid sequence of SEQ ID NO: 1264. In some embodiments, the TCRβV12-3-binding domain consists of an amino acid sequence of SEQ ID NO: 1264.

In some embodiments, the TCRβV12-3-binding domain comprises an scFv. In some embodiments, the TCRβV12-3-binding domain comprises an amino acid sequence with at least about or at most about 75%, 80%, 85%, 90%, 91%, 92%, 93%, 94%, 95%, 96%, 97%, 98%, 99%, or 100% identity to SEQ ID NO: 1270. In some embodiments, the TCRβV12-3-binding domain comprises an amino acid sequence of SEQ ID NO: 1270. In some embodiments, the TCRβV12-3-binding domain consists of an amino acid sequence of SEQ ID NO: 1270.

In some embodiments, the TCRβV12-3-binding domain comprises an scFv. In some embodiments, the TCRβV12-3-binding domain comprises an amino acid sequence with at least about or at most about 75%, 80%, 85%, 90%, 91%, 92%, 93%, 94%, 95%, 96%, 97%, 98%, 99%, or 100% identity to SEQ ID NO: 1271. In some embodiments, the TCRβV12-3-binding domain comprises an amino acid sequence of SEQ ID NO: 1271. In some embodiments, the TCRβV12-3-binding domain consists of an amino acid sequence of SEQ ID NO: 1271.

In some embodiments, the TCRβV12-3-binding domain comprises an scFv. In some embodiments, the TCRβV12-3-binding domain comprises an amino acid sequence with at least about or at most about 75%, 80%, 85%, 90%, 91%, 92%, 93%, 94%, 95%, 96%, 97%, 98%, 99%, or 100% identity to SEQ ID NO: 1275. In some embodiments, the TCRβV12-3-binding domain comprises an amino acid sequence of SEQ ID NO: 1275. In some embodiments, the TCRβV12-3-binding domain consists of an amino acid sequence of SEQ ID NO: 1275.

In some embodiments, the TCRβV12-3-binding domain comprises an scFv. In some embodiments, the TCRβV12-3-binding domain comprises an amino acid sequence with at least about or at most about 75%, 80%, 85%, 90%, 91%, 92%, 93%, 94%, 95%, 96%, 97%, 98%, 99%, or 100% identity to SEQ ID NO: 1276. In some embodiments, the TCRβV12-3-binding domain comprises an amino acid sequence of SEQ ID NO: 1276. In some embodiments, the TCRβV12-3-binding domain consists of an amino acid sequence of SEQ ID NO: 1276.

In some embodiments, the TCRβV12-3-binding domain comprises an scFv. In some embodiments, the TCRβV12-3-binding domain comprises an amino acid sequence with at least about or at most about 75%, 80%, 85%, 90%, 91%, 92%, 93%, 94%, 95%, 96%, 97%, 98%, 99%, or 100% identity to SEQ ID NO: 1277. In some embodiments, the TCRβV12-3-binding domain comprises an amino acid sequence of SEQ ID NO: 1277. In some embodiments, the TCRβV12-3-binding domain consists of an amino acid sequence of SEQ ID NO: 1277.

In some embodiments, the polypeptide molecule comprises a TCRβV12-3-binding domain. In some embodiments, the TCRβV12-3-binding domain comprises a light chain and/or a heavy chain having at least about 75%, 80%, 85%, 90%, 91%, 92%, 93%, 94%, 95%, 96%, 97%, 98%, 99%, or 100% identity to an amino acid sequence of Table 26, Table 27, Table 28, and Table 29. In some embodiments, the polypeptide molecule comprises a heavy chain comprising an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to any one of SEQ ID NOs: 1278, 1426, 1427, 1431-1451, and 1459-1851. In some embodiments, the polypeptide molecule comprises a light chain comprising an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to any one of SEQ ID NOs: 1280, 1287, 1291, 1429, 1430, 1452-1458, and 1852-2229. In some embodiments, the polypeptide molecule comprises: (i) a heavy chain comprising an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to any one of SEQ ID NOs: 1278, 1426, 1427, 1431-1451, and 1459-1851; and (ii) a light chain comprising an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to any one of SEQ ID NOs: 1280, 1287, 1291, 1429, 1430, 1452-1458, and 1852-2229. In some embodiments, the polypeptide molecule comprises a Fab that specifically binds to TCRβV12-3.

In some embodiments, the polypeptide molecule comprises a TCRβV12-3-binding domain. In some embodiments, the TCRβV12-3-binding domain comprises a light chain and/or a heavy chain having at least about 75%, 80%, 85%, 90%, 91%, 92%, 93%, 94%, 95%, 96%, 97%, 98%, 99%, or 100% identity to an amino acid sequence of Table 33. In some embodiments, the polypeptide molecule comprises a heavy chain comprising an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to any one of SEQ ID NOs: 1293-1317, and 1319-1328. In some embodiments, the polypeptide molecule comprises a light chain comprising an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to any one of SEQ ID NOs: 1280-1292. In some embodiments, the polypeptide molecule comprises a Fab that specifically binds to TCRβV12-3. In some embodiments, the polypeptide molecule comprises: (i) a heavy chain comprising an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to any one of SEQ ID NOs: 1293-1317, and 1319-1328; and (ii) a light chain comprising an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to any one of SEQ ID NOs: 1280-1292. In some embodiments, the polypeptide molecule comprises a Fab that specifically binds to TCRβV12-3.

In some embodiments, the TCRβV12-3-binding domain comprises a Fab. In some embodiments, the TCRβV12-3-binding domain comprises: (i) a heavy chain comprising an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to SEQ ID NO: 1315; and (ii) a light chain comprising an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to SEQ ID NO: 1288.

In some embodiments, the TCRβV12-3-binding domain comprises a Fab. In some embodiments, the TCRβV12-3-binding domain comprises: (i) a heavy chain comprising an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to SEQ ID NO: 1317; and (ii) a light chain comprising an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to SEQ ID NO: 1290.

In some embodiments, the TCRβV12-3-binding domain comprises a Fab. In some embodiments, the TCRβV12-3-binding domain comprises: (i) a heavy chain comprising an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to SEQ ID NO: 1322; and (ii) a light chain comprising an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to SEQ ID NO: 1284.

In some embodiments, the TCRβV12-3-binding domain comprises a Fab. In some embodiments, the TCRβV12-3-binding domain comprises: (i) a heavy chain comprising an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to SEQ ID NO: 1297; and (ii) a light chain comprising an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to SEQ ID NO: 1286.

In some embodiments, the TCRβV12-3-binding domain comprises a Fab. In some embodiments, the TCRβV12-3-binding domain comprises: (i) a heavy chain comprising an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to SEQ ID NO: 1323; and (ii) a light chain comprising an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to SEQ ID NO: 1284.

In some embodiments, the TCRβV12-3-binding domain comprises a Fab. In some embodiments, the TCRβV12-3-binding domain comprises: (i) a heavy chain comprising an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to SEQ ID NO: 1327; and (ii) a light chain comprising an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to SEQ ID NO: 1284.

In some embodiments, the TCRβV12-3-binding domain comprises a Fab. In some embodiments, the TCRβV12-3-binding domain comprises: (i) a heavy chain comprising an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to SEQ ID NO: 1328; and (ii) a light chain comprising an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or 100% identical to SEQ ID NO: 1290.

In some embodiments, the TCRβV12-3-binding domain specifically binds to a TCRβV6-5 with high affinity. In some embodiments, a TCRβV12-3-binding domain has a dissociation constant of about 10⁻⁵ to about 10⁻¹² moles per liter or molar (M). For example, a TCRβV12-3-binding domain can have a dissociation constant of about 1000 nM, about 900 nM, about 800 nM, about 700 nM, about 600 nM, about 500 nM, about 400 nM, about 300 nM, about 200 nM, about 100 nM, about 75 nM, about 50 nM, about 25 nM, or about 10 nM, including all ranges and subranges therebetween. In some embodiments, a TCRβV12-3-binding domain has a dissociation constant of less than 200 nM.

### TCRβC-Binding Domains

In some embodiments, the polypeptide molecule comprises a TCRβC-binding domain. In some embodiments, the TCRβC-binding domain selectively binds to human TCRβC (e.g., a TCRβC family, subfamily, or variant thereof). TCRβC families and subfamilies are known in the art, e.g., as described on the ImMunoGeneTics Information System website: http://www.imgt.org/, or in a similar resource.

Provided herein are polypeptide molecules comprising TCRβC-binding domains. In some embodiments, the polypeptide molecule specifically binds TCRβC. In some embodiments, the polypeptide molecule is a bispecific polypeptide molecule and the bispecific polypeptide molecule specifically binds a TAA (e.g., CD19 or PSMA) and TCRβC. In some embodiments, the TCRβC-binding domain comprises a Fab comprising a Fab heavy chain and Fab light chain. In some embodiments, the TCRβC-binding domain is an scFv.

In some embodiments, the polypeptide molecule comprises a TCRβC-binding domain. In some embodiments, the TCRβC-binding domain selectively binds to TCRβC1, or a variant thereof. In some embodiments, the TCRβC-binding domain selectively binds to TCRβC1*01, or a variant thereof. In some embodiments, the TCRβC-binding domain selectively binds to TCRβC1*02, or a variant thereof. In some embodiments, the TCR C-binding domain selectively binds to TCRβC1*03, or a variant thereof.

In some embodiments, the TCRβC-binding domain selectively binds to TCRβC2, or a variant thereof. In some embodiments, the TCRβC-binding domain selectively binds to TCRβC2*01, or a variant thereof. In some embodiments, the TCRβC-binding domain selectively binds to TCRβC2*02, or a variant thereof. In some embodiments, the TCRβC-binding domain selectively binds to TCRβC2*03, or a variant thereof.

Exemplary amino acid sequences for TCRβV family members are shown in Table 4 and can also be found on the ImMunoGeneTics Information System website: www.imgt.org, or in a similar resource.

TABLE 4
Exemplary TCRβC Family Members

| | | | SEQ ID |
|---|---|---|---|
| Target | Species | Amino Acid Sequence | NO: |
| TCRβC1*01 | Human | EDLNKVFPPEVAVFEPSEAEISHTQKATLVCL | 2269 |
| | | ATGFFPDHVELSWWVNGKEVHSGVSTDPQPL | |
| | | KEQPALNDSRYCLSSRLRVSATFWQNPRNHF | |
| | | RCQVQFYGLSENDEWTQDRAKPVTQIVSAEA | |
| | | WGRADCGFTSVSYQQGVLSATILYEILLGKA | |
| | | TLYAVLVSALVLMAMVKRKDF | |
| TCRβC1*01 | Rhesus | EDLKKVFPPKVAVFEPSEAEISHTQKATLVCL | 2270 |
| | Monkey | ATGFYPDHVELSWWVNGKEVHSGVSTDPQP | |
| | | LKEQPALEDSRYCLSSRLRVSATFWHNPRNH | |
| | | FRCQVQFYGLSEDDEWTEDRDKPITQKISAEV | |
| | | WGRADCGFTSVSYQQGVLSATILYEILLGKA | |
| | | TLYAVLVSALMLMAMVKRKDF | |
| TCRβC1*01 | Cynomolgus | EDLKKVFPPKVAVFEPSEAEISHTQKATLVCL | 2271 |
| | Monkey | ATGFYPDHVELSWWVNGKEVHSGVSTDPQP | |
| | | LKEQPALEDSRYCLSSRLRVSATFWHNPRNH | |
| | | FRCQVQFYGLSEDDEWTEDRDKPITQKISAEV | |
| | | WGRACGFTSVSYQQGVLSATILYEILLGKATL | |
| | | YAVLVSALMLMAMVKRKDF | |
| TCRβC1*02 | Human | EDLNKVFPPEVAVFEPSEAEISHTQKATLVCL | 2269 |
| | | ATGFFPDHVELSWWVNGKEVHSGVSTDPQPL | |
| | | KEQPALNDSRYCLSSRLRVSATFWQNPRNHF | |
| | | RCQVQFYGLSENDEWTQDRAKPVTQIVSAEA | |
| | | WGRADCGFTSVSYQQGVLSATILYEILLGKA | |
| | | TLYAVLVSALVLMAMVKRKDF | |
| TCRβC1*02 | Rhesus | EDLKKVFPPKVAVFEPSEAEISHTQKATLVCL | 2272 |
| | Monkey | ATGFYPDHVELSWWVNGKEVHSGVSTDPQP | |
| | | LKEQPALEDSRYCLSSRLRVSATFWHNPRNH | |
| | | FRCQVQFYGLSEDDEWTEDRDKPITQKISAEV | |
| | | WGRADCGFTSVSYQQGVLSATILYEILLGKAS | |
| | | LYAVLVSALMLMAMVKRKDF | |
| TCRβC1*03 | Human | EDLNKVFPPEVAVFEPSEAEISHTQKATLVCL | 2269 |
| | | ATGFFPDHVELSWWVNGKEVHSGVSTDPQPL | |
| | | KEQPALNDSRYCLSSRLRVSATFWQNPRNHF | |
| | | RCQVQFYGLSENDEWTQDRAKPVTQIVSAEA | |
| | | WGRADCGFTSVSYQQGVLSATILYEILLGKA | |
| | | TLYAVLVSALVLMAMVKRKDF | |
| TCRβC2*01 | Human | EDLKNVFPPEVAVFEPSEAEISHTQKATLVCL | 2273 |
| | | ATGFYPDHVELSWWVNGKEVHSGVSTDPQP | |
| | | LKEQPALNDSRYCLSSRLRVSATFWQNPRNH | |
| | | FRCQVQFYGLSENDEWTQDRAKPVTQIVSAE | |
| | | AWGRADCGFTSESYQQGVLSATILYEILLGK | |
| | | ATLYAVLVSALVLMAMVKRKDSRG | |
| TCRβC2*01 | Rhesus | EDLKKVFPPKVAVFEPSEAEISHTQKATLVCL | 2274 |
| | Monkey | ATGFYPDHVELSWWVNGKEVHSGVSTDPQP | |
| | | LKEQPTLEDSRYCLSSRLRVSATFWHNPRNHF | |
| | | RCQVQFYGLSEDDEWTEDRDKPITQKISAEA | |
| | | WGRADCGFTSESYQQGVLSATILYEILLGKAT | |
| | | LYAVLVSALVLMAMVKRKDS | |
| TCRβC2*01 | Cynomolgus | EDLKKVFPPKVAVFEPSEAEISHTQKATLVCL | 2275 |
| | Monkey | ATGFYPDHVELSWWVNGKEVHSGVSTDPQP | |
| | | LKEQPALEDSRYCLSSRLRVSATFWHNPRNH | |
| | | FRCQVQFYGLSEDDEWTEDRDKPITQKISAEA | |
| | | WGRADCGFTSESYQQGVLSATILYEILLGKAT | |
| | | LYAVLVSALVLMAMVKRKDS | |
| TCRβC2*02 | Human | EDLKNVFPPEVAVFEPSEAEISHTQKATLVCL | 2273 |
| | | ATGFYPDHVELSWWVNGKEVHSGVSTDPQP | |
| | | LKEQPALNDSRYCLSSRLRVSATFWQNPRNH | |
| | | FRCQVQFYGLSENDEWTQDRAKPVTQIVSAE | |
| | | AWGRADCGFTSESYQQGVLSATILYEILLGK | |
| | | ATLYAVLVSALVLMAMVKRKDSRG | |
| TCRβC2*03 | Human | DLKNVFPPKVAVFEPSEAEISHTQKATLVCLA | 2276 |
| | | TGFYPDHVELSWWVNGKEVHSGVSTDPQPL | |
| | | KEQPALNDSRYCLSSRLRVSATFWQNPRNHF | |
| | | RCQVQFYGLSENDEWTQDRAKPVTQIVSAEA | |
| | | WGRATVASPPESYQQGVLSATILYEILLGKAT | |
| | | LYAVLVSALVLMAMVKRKDSRG | |
| TCRβC2*03 | Rhesus | EDLKKVFPPKVAVFEPSEAEISHTQKATLVCL | 2277 |
| | Monkey | ATGFYPDHVELSWWVNGKEVHSGVSTDPQP | |
| | | LKEQPTLEDSRYCLSSRLRVSATFWHNPRNHF | |
| | | RCQVQFYGLSEDDEWTEDRDKPITQKISAEA | |
| | | WGRADCGFTSESYQQGVLSATILYEILLGKAT | |
| | | LYAVLVSALVLMAMVKRKDS | |

In some embodiments, the TCRβC-binding domain comprises: (a) an immunoglobulin heavy chain variable (VH) region comprising a heavy chain complementarity determining region (CDR) 1 (HCDR1), a heavy chain CDR2 (HCDR2), and a heavy chain CDR3 (HCDR3); and (b) an immunoglobulin light chain variable (VL) region comprising a light chain complementarity determining region (CDR) 1 (LCDR1), a light chain CDR2 (LCDR2), and a light chain CDR3 (LCDR3). In some embodiments, the polypeptide molecule comprises a TCRβV-binding domain comprising: (a) a VH region comprising a HCDR1, a HCDR2, and a HCDR3; and (b) a VL region comprising a LCDR1, a LCDR2, and a LCDR3.

Exemplary TCRβC-binding domains are provided in Table 5.

TABLE 5
Exemplary TCRβC-Binding Domains

| | | | SEQ ID |
|---|---|---|---|
| Domain | | Amino Acid Sequence | NO |
| TCRβC1 | HCDR1 (IMGT) | GYTFTGYV | 2278 |
| | HCDR2 (IMGT) | INPYNDDI | 2279 |
| | HCDR3 (IMGT) | ARGAGYNFDGAYRFFDF | 2280 |
| | LCDR1 (IMGT) | QRLVHSNGNTY | 2281 |
| | LCDR2 (IMGT) | RVS | |
| | LCDR3 (IMGT) | SQSTHVPYT | 2282 |
| | HCDR1 (Kabat) | GYVMH | 2283 |
| | HCDR2 (Kabat) | FINPYNDDIQSNERFRG | 2284 |
| | HCDR3 (Kabat) | GAGYNFDGAYRFFDF | 2285 |
| | LCDR1 (Kabat) | RSSQRLVHSNGNTYLH | 2286 |
| | LCDR2 (Kabat) | RVSNRFP | 2287 |
| | LCDR3 (Kabat) | SQSTHVPYT | 2282 |
| | VH region | QVQLVQSGAEVKKPGASVKVSCKASGYT | 2288 |
| | | FTGYVMHWVRQAPGQGLEWMGFINPYN | |
| | | DDIQSNERFRGRVTMTRDTSISTAYMELSR | |
| | | LRSDDTAVYYCARGAGYNFDGAYRFFDF | |
| | | WGQGTMVTVSS | |
| | VL region | DIVMTQSPLSLPVTPGEPASISCRSSQRLVH | 2289 |
| | | SNGNTYLHWYLQKPGQSPRLLIYRVSNRF | |
| | | PGVPDRFSGSGSGTDFTLKISRVEAEDVGV | |
| | | YYCSQSTHVPYTFGQGTKLEIK | |
| TCRβC2 | HCDR1 (IMGT) | GYKFTGFV | 2290 |
| | HCDR2 (IMGT) | INPYNDDI | 2279 |
| | HCDR3 (IMGT) | ARGNGYNFDGAYRFFDF | 2291 |
| | LCDR1 (IMGT) | QRLVHSNGNTY | 2281 |
| | LCDR2 (IMGT) | RVS | |
| | LCDR3 (IMGT) | SQSTHVPYT | 2282 |
| | HCDR1 (Kabat) | GFVMH | 2292 |
| | HCDR2 (Kabat) | FINPYNDDIQSNERFRG | 2284 |
| | HCDR3 (Kabat) | GNGYNFDGAYRFFDF | 2293 |
| | LCDR1 (Kabat) | RSSQRLVHSNGNTYLH | 2286 |
| | LCDR2 (Kabat) | RVSNRFP | 2287 |
| | LCDR3 (Kabat) | SQSTHVPYT | 2282 |
| | VH region | QVQLVQSGAEVKKPGASVKVSCKASGYK | 2294 |
| | | FTGFVMHWVRQAPGQGLEWMGFINPYND | |
| | | DIQSNERFRGRVTMTRDTSISTAYMELSRL | |
| | | RSDDTAVYYCARGNGYNFDGAYRFFDFW | |
| | | GQGTMVTVSS | |
| | VL region | DIVMTQSPLSLPVTPGEPASISCRSSQRLVH | 2289 |
| | | SNGNTYLHWYLQKPGQSPRLLIYRVSNRF | |
| | | PGVPDRFSGSGSGTDFTLKISRVEAEDVGV | |
| | | YYCSQSTHVPYTFGQGTKLEIK | |

In some embodiments, the polypeptide molecule comprises a TCRβC1-binding domain. In some embodiments, the TCRβC1-binding domain comprises, according to IMGT, an HCDR1 comprising SEQ ID NO: 2278, an HCDR2 comprising SEQ ID NO: 2279, an HCDR3 comprising SEQ ID NO: 2280, an LCDR1 comprising SEQ ID NO: 2281, an LCDR2 comprising RVS, and an LCDR3 comprising SEQ ID NO: 2282.

In some embodiments, the polypeptide molecule comprises a TCRβC1-binding domain. In some embodiments, the TCRβC1-binding domain comprises, according to Kabat, a HCDR1 comprising SEQ ID NO: 2283, a HCDR2 comprising SEQ ID NO: 2284, a HCDR3 comprising SEQ ID NO: 2285, a LCDR1 comprising SEQ ID NO: 2286, a LCDR2 comprising SEQ ID NO: 2287, and a LCDR3 comprising SEQ ID NO: 2282.

In some embodiments, the TCRβC1-binding domain comprises a VH comprising an amino acid sequence with at least about 85%, 90%, 95%, 96%, 97%, 98%, 99%, or 1000% identity to SEQ ID NO: 2288; and a VL comprising an amino acid sequence with at least about 85%, 90%, 95%, 96%, 97%, 98%, 99%, or 100% identity to SEQ ID NO: 2289. In some embodiments, the TCRβC1-binding domain comprises a VH comprising an amino acid sequence of SEQ ID NO: 2288; and a VL comprising an amino acid sequence of SEQ ID NO: 2289. In some embodiments, the TCRβC1-binding domain comprises an scFv. In some embodiments, the TCRβC1-binding domain comprises a Fab.

In some embodiments, the polypeptide molecule comprises a TCRβC2-binding domain. In some embodiments, the TCRβC2-binding domain comprises, according to IMGT, a HCDR1 comprising SEQ ID NO: 2290, a HCDR2 comprising SEQ ID NO: 2279, a HCDR3 comprising SEQ ID NO: 2291, a LCDR1 comprising SEQ ID NO: 2281, a LCDR2 comprising RVS, and a LCDR3 comprising SEQ ID NO: 2282.

In some embodiments, the polypeptide molecule comprises a TCRβC2-binding domain. In some embodiments, the TCRβC2-binding domain comprises, according to Kabat, a HCDR1 comprising SEQ ID NO: 2292, a HCDR2 comprising SEQ ID NO: 2284, a HCDR3 comprising SEQ ID NO: 2293, a LCDR1 comprising SEQ ID NO: 2286, a LCDR2 comprising SEQ ID NO: 2287, and a LCDR3 comprising SEQ ID NO: 2282.

In some embodiments, the TCRβC2-binding domain comprises a VH comprising an amino acid sequence with at least about 85%, 90%, 95%, 96%, 97%, 98%, 99%, or 100% identity to SEQ ID NO: 2294; and a VL comprising an amino acid sequence with at least about 85%, 90%, 95%, 96%, 97%, 98%, 99%, or 100% identity to SEQ ID NO: 2289. In some embodiments, the TCRβC2-binding domain comprises a VH comprising an amino acid sequence of SEQ ID NO: 2294; and a VL comprising an amino acid sequence of SEQ ID NO: 2289. In some embodiments, the TCRβC2-binding domain comprises scFv. In some embodiments, the TCRβC2-binding domain comprises a Fab.

In some embodiments, the TCRβC1-binding domain comprises an amino acid sequence with at least about 75%, 80%, 85%, 90%, 91%, 92%, 93%, 94%, 95%, 96%, 97%, 98%, 99%, or 100% identity to SEQ ID NO: 2297. In some embodiments, the TCRβC1-binding domain and the polypeptide molecule comprises an amino acid sequence of SEQ ID NO: 2297. In some embodiments, the TCRβC1-binding domain consists of an amino acid sequence of SEQ ID NO: 2297. In some embodiments, the TCRβC1-binding domain comprises an scFv.

In some embodiments, the TCRβC2-binding domain comprises an amino acid sequence with at least about 75%, 80%, 85%, 90%, 91%, 92%, 93%, 94%, 95%, 96%, 97%, 98%, 99%, or 100% identity to SEQ ID NO: 2298. In some embodiments, the TCRβC2-binding domain comprises an amino acid sequence of SEQ ID NO: 2298. In some embodiments, the TCRβC2-binding domain consists of an amino acid sequence of SEQ ID NO: 2298. In some embodiments, the TCRβC2-binding domain comprises an scFv.

In some embodiments, the TCRβC-binding domain specifically binds to a TCRβC with high affinity. In some embodiments, a TCRβC-binding domain has a dissociation constant of about 10⁻⁵ to about 10⁻¹² moles per liter or molar (M). For example, a TCRβC-binding domain can have a dissociation constant of about 1000 nM, about 900 nM, about 800 nM, about 700 nM, about 600 nM, about 500 nM, about 400 nM, about 300 nM, about 200 nM, about 100 nM, about 75 nM, about 50 nM, about 25 nM, or about 10 nM, including all ranges and subranges therebetween. In some embodiments, a TCRβC-binding domain has a dissociation constant of less than 200 nM.

### Tumor-Associated Antigen (TAA) Binding Domains

Provided herein are polypeptide molecules comprising tumor-associated antigen (TAA) binding domains. In some embodiments, the polypeptide molecule specifically binds a TAA. In some embodiments, the polypeptide molecule is a bispecific polypeptide molecule and the bispecific polypeptide molecule specifically binds a TAA (e.g., CD19 or PSMA) and TCRβV (e.g., TCRβV6-5 and TCRβV12-3). Thus, in some embodiments, the polypeptide molecule comprises a T-cell binding domain and a TAA-binding domain. Persons having skill in the art will be able to identify and select TAA binding domains. In some embodiments, the polypeptide molecules of the present disclosure are compatible with any TAA. In some embodiments, the TAA-binding domain is a CD19-binding domain. In some embodiments, the TAA-binding domain is a PSMA-binding domain. In some embodiments, the TAA-binding domain comprises a Fab comprising a Fab heavy chain and Fab light chain. In some embodiments, the TAA-binding domain is an scFv.

In some embodiments, the TAA-binding domain specifically binds to a TAA with high affinity. In some embodiments, the TAA-binding domain has a dissociation constant (K_{D}) of about 10⁻⁵ to about 10⁻¹² moles per liter or molar (M). For example, In some embodiments, the TAA-binding domain has a dissociation constant of about 1000 nM, about 900 nM, about 800 nM, about 700 nM, about 600 nM, about 500 nM, about 400 nM, about 300 nM, about 200 nM, about 100 nM, about 75 nM, about 50 nM, about 25 nM, or about 10 nM, including all ranges and subranges therebetween. In some embodiments, the TAA-binding domain has a dissociation constant of less than 200 nM.

### CD19-Binding Domains

Provided herein are polypeptide molecules comprising CD19-binding domains. In some embodiments, the polypeptide molecule specifically binds CD19 antigen. In some embodiments, the polypeptide molecule is a bispecific polypeptide molecule and the bispecific polypeptide molecule specifically binds CD19 and TCRβV (e.g., TCRβV6-5 or TCRβV12-3). In some embodiments, the CD19-binding domain is a tumor-associated antigen (TAA)-binding domain.

Exemplary CD19-binding domains are provided in Table 6.

TABLE 6
Exemplary CD19-Binding Domains

| | SEQ ID | | | | | | | |
|---|---|---|---|---|---|---|---|---|
| | NO | | Kabat CDRs | | | | | |
| ID | VL | VH | CDRL1 | CDRL2 | CDRL3 | CDRH1 | CDRH2 | CDRH3 |
| CD19- | 1343 | 1345 | RSSKSLQN | RMSNL | MQHLEY | SYVM | YINPYN | GTYYY |
| Binding | | | VNGNTYL | NS | PIT | H (SEQ | DGTKY | GTRVF |
| Domain 1 | | | Y (SEQ ID | (SEQ ID | (SEQ ID | ID NO: | NEKFQ | DY |
| | | | NO: 1347) | NO: | NO: 1351) | 1353) | G (SEQ | (SEQ ID |
| | | | | 1349) | | | ID NO: | NO: |
| | | | | | | | 1355) | 1357) |
| CD19- | 1344 | 1346 | SASSSVGY | DTSKLA | FQGSVY | TSTMG | FIWWD | MELW |
| Binding | | | MH | F (SEQ | PFT | VG | DDKRY | SYYFD |
| Domain 2 | | | (SEQ ID | ID NO: | (SEQ ID | (SEQ ID | NPNLKS | P (SEQ |
| | | | NO: 1348) | 1350) | NO: 1352) | NO: | (SEQ ID | ID NO: |
| | | | | | | 1354) | NO: | 1358) |
| | | | | | | | 1356) | |
| | SEQ ID | | | | | | | |
| | NO | | IMGT CDRs | | | | | |
| ID | VL | VH | CDRL1 | CDRL2 | CDRL3 | CDRH1 | CDRH2 | CDRH3 |
| CD19- | 1343 | 1345 | KSLQNVN | RMS | MQHLEY | GYTFT | INPYND | ARGTY |
| Binding | | | GNTY | | PIT | SYV | GT | YYGTR |
| Domain 1 | | | (SEQ ID | | (SEQ ID | (SEQ ID | (SEQ ID | VFDY |
| | | | NO: 1359) | | NO: 1351) | NO: | NO: | (SEQ ID |
| | | | | | | 1361) | 1363) | NO: |
| | | | | | | | | 1365) |
| CD19- | 1344 | 1346 | SSVGY | DTS | FQGSVY | GGSIST | IWWDD | ARMEL |
| Binding | | | (SEQ ID | | PFT | STMG | DK (SEQ | WSYYF |
| Domain 2 | | | NO: 1360) | | (SEQ ID | (SEQ ID | ID NO: | DP |
| | | | | | NO: 1352) | NO: | 1364) | (SEQ ID |
| | | | | | | 1362) | | NO: |
| | | | | | | | | 1366) |

In some embodiments, the CD19-binding domain comprises a VL comprising a CDRL1, a CDRL2, a CDRL3; and a VH comprising a CDRH1, a CDRH2, and a CDRH3, wherein the CDRL1 comprises RSSKSLQNVNGNTYLY (SEQ ID NO: 1347), the CDRL2 comprises RMSNLNS (SEQ ID NO: 1349), the CDRL3 comprises MQHLEYPIT (SEQ ID NO: 1351), the CDRH1 comprises SYVMH (SEQ ID NO: 1353), the CDRH2 comprises Y₁NPYNIDGTKYNEKFQG (SEQ ID NO: 1355), and the CDRH3 comprises GTYYYGTRVFDY (SEQ ID NO: 1357). In some embodiments, the CDRs comprise 1, 2, 3, or more amino acid substitutions. In some embodiments, the CD19-binding domain comprises an scFv. In some embodiments, the CD19-binding domain comprises a Fab.

In some embodiments, the CD19-binding domain comprises a VL comprising a CDRL1, a CDRL2, a CDRL3; and a VH comprising a CDRH1, a CDRH2, and a CDRH3, wherein the CDRL1 comprises KSLQNVNGNTY (SEQ ID NO: 1359), the CDRL2 comprises RMS, the CDRL3 comprises MQTILEYPIT (SEQ ID NO: 1351), the CDRH1 comprises GYTFTSYV (SEQ ID NO: 1361), the CDRH2 comprises INPYNDGT (SEQ ID NO: 1363), and the CDRH3 comprises ARGTYYYGTRVFDY (SEQ ID NO: 1365). In some embodiments, the CDRs comprise 1, 2, 3, or more amino acid substitutions. In some embodiments, the CD19-binding domain comprises an scFv. In some embodiments, the CD19-binding domain comprises a Fab.

In some embodiments, the CD19-binding domain comprises a VH comprising an amino acid sequence with at least about 85%, 90%, 95%, 96%, 97%, 98%, 99%, or 100% identity to SEQ ID NO: 1345; and a VL comprising an amino acid sequence with at least about 85%, 90%, 95%, 96%, 97%, 98%, 99%, or 100% identity to SEQ ID NO: 1343. In some embodiments, the CD19-binding domain comprises a VH comprising an amino acid sequence of SEQ ID NO: 1345; and a VL comprising an amino acid sequence of SEQ ID NO: 1343. In some embodiments, the CD19-binding domain comprises an scFv. In some embodiments, the CD19-binding domain comprises a Fab.

In some embodiments, the CD19-binding domain comprises a VH comprising an amino acid sequence with at least about 85%, 90%, 95%, 96%, 97%, 98%, 99%, or 100% identity to SEQ ID NO: 1345; and a VL comprising an amino acid sequence with at least about 85%, 90%, 95%, 96%, 97%, 98%, 99%, or 100% identity to SEQ ID NO: 1343. In some embodiments, the CD19-binding domain comprises a VH comprising an amino acid sequence of SEQ ID NO: 1345; and a VL comprising an amino acid sequence of SEQ ID NO: 1343. In some embodiments, the CD19-binding domain comprises an scFv. In some embodiments, the CD19-binding domain comprises a Fab.

In some embodiments, the CD19-binding domain comprises a VL comprising a CDRL1, a CDRL2, a CDRL3; and a VH comprising a CDRH1, a CDRH2, and a CDRH3, wherein CDRL1 comprises SASSSVGYMH (SEQ ID NO: 1348), CDRL2 comprises DTSKLAF (SEQ ID NO: 1350), CDRL3 comprises FQGSVYPFT (SEQ ID NO: 1352), CDRH1 comprises TSTMGVG (SEQ ID NO: 1354), CDRH2 comprises FIWWDDDKRYNPNLKS (SEQ ID NO: 1356), and CDRH3 comprises MELWSYYFDP (SEQ ID NO: 1358). In some embodiments, the CD19-binding domain comprises an scFv. In some embodiments, the CD19-binding domain comprises a Fab.

In some embodiments, the CD19-binding domain comprises a VL comprising a CDRL1, a CDRL2, a CDRL3; and a VH comprising a CDRH1, a CDRH2, and a CDRH3, wherein CDRL1 comprises SSVGY (SEQ ID NO: 1360), CDRL2 comprises DTS, CDRL3 comprises FQGSVYPFT (SEQ ID NO: 1352), CDRH1 comprises GGSISTSTMG (SEQ ID NO: 1362), CDRH2 comprises IWWDDDK (SEQ ID NO: 1364), and CDRH3 comprises ARMELWSYYFDP (SEQ ID NO: 1366). In some embodiments, a polypeptide molecule comprises the CD19-binding domain and the polypeptide molecule is an scFv. In some embodiments, a polypeptide molecule comprises the CD19-binding domain and the polypeptide molecule comprises a Fab.

In some embodiments, the CD19-binding domain comprises a VH comprising an amino acid sequence with at least about 75%, 80%, 85%, 90%, 91%, 92%, 93%, 94%, 95%, 96%, 97%, 98%, 99%, or 100% identity to SEQ ID NO: 1346; and a VL comprising an amino acid sequence with at least about 75%, 80%, 85%, 90%, 91%, 92%, 93%, 94%, 95%, 96%, 97%, 98%, 99%, or 100% identity to SEQ ID NO: 1344. In some embodiments, the CD19-binding domain comprises a VH comprising an amino acid sequence of SEQ ID NO: 1346; and a VL comprising an amino acid sequence of SEQ ID NO: 1344. In some embodiments, the CD19-binding domain comprises an scFv. In some embodiments, the CD19-binding domain comprises a Fab.

In some embodiments, the CD19-binding domain comprises an amino acid sequence with at least about 75%, 80%, 85%, 90%, 91%, 92%, 93%, 94%, 95%, 96%, 97%, 98%, 99%, or 100% identity to SEQ ID NO: 1367. In some embodiments, the CD19-binding domain and the polypeptide molecule comprises an amino acid sequence of SEQ ID NO: 1367. In some embodiments, the CD19-binding domain consists of an amino acid sequence of SEQ ID NO: 1367. In some embodiments, the CD19-binding domain comprises an scFv.

In some embodiments, the CD19-binding domain comprises a light chain comprising an amino acid sequence with at least about 75%, 80%, 85%, 90%, 91%, 92%, 93%, 94%, 95%, 96%, 97%, 98%, 99%, or 100% identity to SEQ ID NO: 1368; and a heavy chain comprising an amino acid sequence with at least about 75%, 80%, 85%, 90%, 91%, 92%, 93%, 94%, 95%, 96%, 97%, 98%, 99%, or 100% identity to SEQ ID NO: 1370. In some embodiments, the CD19-binding domain comprises a light chain comprising an amino acid sequence of SEQ ID NO: 1368; and a heavy chain comprising an amino acid sequence of SEQ ID NO: 1370. In some embodiments, the CD19-binding domain comprises a light chain consisting of an amino acid sequence of SEQ ID NO: 1368; and a heavy chain consisting of an amino acid sequence of SEQ ID NO: 1370. In some embodiments, the CD19-binding domain comprises a Fab.

In some embodiments, the CD19-binding domain comprises a light chain comprising an amino acid sequence with at least about 75%, 80%, 85%, 90%, 91%, 92%, 93%, 94%, 95%, 96%, 97%, 98%, 99%, or 100% identity to SEQ ID NO: 1369; and a heavy chain comprising an amino acid sequence with at least about 75%, 80%, 85%, 90%, 91%, 92%, 93%, 94%, 95%, 96%, 97%, 98%, 99%, or 100% identity to SEQ ID NO: 1371. In some embodiments, the CD19-binding domain comprises a light chain comprising an amino acid sequence of SEQ ID NO: 1369; and a heavy chain comprising an amino acid sequence of SEQ ID NO: 1371. In some embodiments, the CD19-binding domain comprises a light chain consisting of an amino acid sequence of SEQ ID NO: 1369; and a heavy chain consisting of an amino acid sequence of SEQ ID NO: 1371. In some embodiments, the CD19-binding domain comprises a Fab.

In some embodiments, the CD19-binding domain specifically binds to CD19 with high affinity. In some embodiments, the CD19-binding domain has a dissociation constant of about 10⁻⁵ to about 10⁻¹² moles per liter or molar (M). For example, the CD19-binding domain has a dissociation constant of about 1000 nM, about 900 nM, about 800 nM, about 700 nM, about 600 nM, about 500 nM, about 400 nM, about 300 nM, about 200 nM, about 100 nM, about 75 nM, about 50 nM, about 25 nM, or about 10 nM, including any range or subrange therebetween. In some embodiments, the CD19-binding domain has a dissociation constant of less than 200 nM.

### PSMA-Binding Domains

Provided herein are polypeptide molecules comprising PSMA-binding domains. In some embodiments, the polypeptide molecule specifically binds PSMA antigen. In some embodiments, the polypeptide molecule is a bispecific polypeptide molecule and the bispecific polypeptide molecule specifically binds PSMA and TCRβV (e.g., TCRβV6-5 or TCRβV12-3). In some embodiments, the PSMA-binding domain is an scFv. In some embodiments, the PSMA-binding domain comprises a Fab comprising a Fab heavy chain and Fab light chain. In some embodiments, the PSMA-binding domain is a tumor-associated antigen (TAA) binding domain.

In some embodiments, the polypeptide molecule comprises a prostate-specific membrane antigen (PSMA) binding domain that specifically binds to PSMA (e.g., human PSMA). PSMA is also known as glutamate carboxypeptidase II (GCPII), N-acetyl-L-aspartyl-L-glutamate peptidase I (NAALADase 1), or NAAG peptidase. PSMA is a membrane glycoprotein and zinc metallopeptidase. Human PSMA is highly expressed in the prostate, roughly a hundred times greater than in most other tissues. In some prostate cancers, PSMA is expressed at approximately 100-1000-fold higher levels compared to healthy, noncancerous prostate tissue. High levels of PSMA expression in prostate cancer is associated with neovasculature in malignant tissue and an increased likelihood of disease progression.

In some embodiments, the PSMA-binding domain specifically binds to a PSMA with high affinity. In some embodiments, the PSMA-binding domain has a dissociation constant (K_{D}) of about 10⁻⁵ to about 10⁻¹² moles per liter or molar (M). For example, the PSMA-binding domain has a dissociation constant of about 1000 nM, about 900 nM, about 800 nM, about 700 nM, about 600 nM, about 500 nM, about 400 nM, about 300 nM, about 200 nM, about 100 nM, about 75 nM, about 50 nM, about 25 nM, or about 10 nM, including all ranges and subranges therebetween. In some embodiments, the PSMA-binding domain has a dissociation constant of less than 200 nM.

In some embodiments, the polypeptide molecule comprises a PSMA-binding domain, and the polypeptide molecule is an antibody selected from the group consisting of: 7E11, J591, MLN591, J415, J533, and E99, or a fragment or variant thereof. In some embodiments, the PSMA-binding domain comprises a VH region from an antibody selected from the group consisting of: 7E11, J591, MLN591, J415, J533, and E99, or a fragment or variant thereof. In some embodiments, the PSMA-binding domain comprises a VL region from an antibody selected from the group consisting of: 7E11, J591, MLN591, J415, J533, and E99, or a fragment or variant thereof. In some embodiments, the PSMA-binding domain comprises a VH region and a VL region from an antibody selected from the group consisting of: 7E11, J591, MLN591, J415, J533, and E99, or a fragment or variant thereof. In some embodiments, the PSMA-binding domain comprises CDRs from an antibody selected from the group consisting of: 7E11, J591, MLN591, J415, J533, and E99, or a fragment or variant thereof. In some embodiments, the CDRs comprise 1, 2, 3, or more amino acid substitutions.

In some embodiments, the PSMA-binding domain comprises a VL comprising a CDRL1, a CDRL2, a CDRL3; and a VH comprising a CDRH1, a CDRH2, and a CDRH3, wherein the CDRL1 comprises RASQGISNYLA (SEQ ID NO: 2306), the CDRL2 comprises EASTLQS (SEQ ID NO: 2307), the CDRL3 comprises QNYNSAPFT (SEQ ID NO: 2302), the CDRH1 comprises RYGMH (SEQ ID NO: 2303), the CDRH2 comprises VIWYDGSNKYYADSVKG (SEQ ID NO: 2304), and the CDRH3 comprises GGDFLYYYYYGMDV (SEQ ID NO: 2305). In some embodiments, the CDRs comprise 1, 2, 3, or more amino acid substitutions. In some embodiments, the PSMA-binding domain comprises an scFv. In some embodiments, the PSMA-binding domain comprises a Fab.

In some embodiments, the PSMA-binding domain comprises a VL comprising a CDRL1, a CDRL2, a CDRL3; and a VH comprising a CDRH1, a CDRH2, and a CDRH3, wherein the CDRL1 comprises QGISNY (SEQ ID NO: 2260), the CDRL2 comprises EAS, the CDRL3 comprises QNYNSAPFT (SEQ ID NO: 2302), the CDRH1 comprises GFAFSRYG (SEQ ID NO: 2299), the CDRH2 comprises IWYDGSNK (SEQ ID NO: 2300), and the CDRH3 comprises ARGGDFLYYYYYGMDV (SEQ ID NO: 2301). In some embodiments, the CDRs comprise 1, 2, 3, or more amino acid substitutions. In some embodiments, the PSMA-binding domain comprises an scFv. In some embodiments, the PSMA-binding domain comprises a Fab.

In some embodiments, the PSMA-binding domain comprises a heavy chain comprising an amino acid sequence with at least about 75%, 80%, 85%, 90%, 91%, 92%, 93%, 94%, 95%, 96%, 97%, 98%, 99%, or 100% identity to SEQ ID NO: 57; and a light chain comprising an amino acid sequence with at least about 75%, 80%, 85%, 90%, 91%, 92%, 93%, 94%, 95%, 96%, 97%, 98%, 99%, or 100% identity to SEQ ID NO: 54. In some embodiments, the PSMA-binding domain comprises a heavy chain comprising an amino acid sequence of SEQ ID NO: 57; and a light chain comprising an amino acid sequence of SEQ ID NO: 54. In some embodiments, the PSMA-binding domain comprises a heavy chain consisting of an amino acid sequence of SEQ ID NO: 57; and a light chain consisting of an amino acid sequence of SEQ ID NO: 54. In some embodiments, the PSMA-binding domain comprises a Fab.

### Linkers

In some embodiments, the present disclosure provides polypeptide molecules comprising a linker. In some embodiments the polypeptide molecules comprise 2, 3, 4, 5, 6, 7, 8, 9, or 10 linkers, including any ranges and subranges therebetween. In some embodiments, the polypeptide molecule may contain two or more of the same linkers. In some embodiments, each linker within the polypeptide molecule is different. For example, in some embodiments, the polypeptide molecule comprises a linker connecting the VH region and VL region of the T cell-binding domain. In some embodiments, the polypeptide molecule comprises a linker connecting the T cell-binding domain to the TAA-binding domain. Persons having skill in the art will be able to select suitable linkers for each portion of the polypeptide molecule. Information regarding the possible any one of the one or more linkers within the polypeptide molecules is provided below.

Suitable linkers are generally those which allow each polypeptide component of a binder described herein to fold with a three-dimensional structure very similar to the polypeptide components produced individually without any linker or other component. Linkers include single amino acids, peptides, polypeptides, non-proteinaceous polymers such as polyethylene glycol (PEG), polypropylene glycol, polyoxyalkylenes, or copolymers of polyethylene glycol and polypropylene glycol, and chemical linkers.

In some embodiments, the linker is derived from an immunoglobulin light chain, for example Cκ or Cλ. In some embodiments, the linker is derived from an immunoglobulin heavy chain of any isotype, including, for example, Cγ1, Cγ2, Cγ3, Cγ4, Cα1, Cα2, Cδ, Cε, and Cμ. Linker sequences can also be derived from other proteins such as Ig-like proteins (e.g., TCR, FcR, KIR), hinge region-derived sequences, and other natural sequences from other proteins.

In some embodiments, the linker comprises any of the following amino acid residues: Gly, Ser, Ala, Pro, or Thr. Exemplary linkers include, but are not limited to, glycine-serine linkers, glycine-alanine linkers, proline-threonine, and alanine-serine linkers.

In some embodiments, the linker is a length that is adequate to link two polypeptide molecules together in such a way that they assume the correct conformation relative to one another so that they retain the desired activity. In some embodiments, the linker is between 5 amino acids and 50 amino acids in length, including any ranges and subranges therebetween. In some embodiments, the linker is between about 5 amino acids to about 10 amino acids in length. In some embodiments, the linker is between about 5 amino acids to about 15 amino acids in length. In some embodiments, the linker is between about 5 amino acids to about 20 amino acids in length. In some embodiments, the linker is between about 5 amino acids to about 30 amino acids in length. In some embodiments, the linker is between about 10 amino acids to about 15 amino acids in length. In some embodiments, the linker is between about 10 amino acids to about 20 amino acids in length. In some embodiments, the linker is between about 10 amino acids to about 30 amino acids in length. In some embodiments, the linker is between about 15 amino acids to about 30 amino acids in length. In some embodiments, the linker is between about 15 amino acids to about 45 amino acids in length.

In some embodiments, the linker is 5 amino acids in length. In some embodiments, the linker is 10 amino acids in length. In some embodiments, the linker is 15 amino acids in length. In some embodiments, the linker is 20 amino acids in length. In some embodiments, the linker is 25 amino acids in length. In some embodiments, the linker is 30 amino acids in length. In some embodiments, the linker is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length, including any ranges and subranges therebetween.

In some embodiments, the linker comprises at least 2 (e.g., 5, 10, 15, 20, 40, 60 or more) amino acids which result in a flexible or semi-flexible linkage between adjacent domains in a single polypeptide molecule.

In some embodiments, a linker comprises: GGGGSGGGGSGGGGS (SEQ ID NO: 1342). In some embodiments, a linker comprises: (G)n, n=1-30 (SEQ ID NO: 2332). In some embodiments, a linker comprises: (GxS)n, x=1-5, n=15 (SEQ ID NO: 2333). In some embodiments, a linker comprises: (SxG)n, x=1-5, n=15 (SEQ ID NO: 2334). In some embodiments, a linker comprises: (GSGGS)n, n=1-5 (SEQ ID NO: 2335). In some embodiments, a linker comprises: (GGGGS)n, n=1-5 (SEQ ID NO: 2336). In some embodiments, a linker comprises: GSGSG (SEQ ID NO: 2337). In some embodiments, a linker comprises: GGSGGGG (SEQ ID NO: 2338). In some embodiments, a linker comprises: GGGGS (SEQ ID NO: 1425). In some embodiments, a linker comprises: GGGGSGGGGS (SEQ ID NO: 2339). In some embodiments, a linker comprises: GGGGSGGGGSGGGGSGGGGS (SEQ ID NO: 2340). In some embodiments, a linker comprises: GGGGSGGGGSGGGGSGGGGSGGGGS (SEQ ID NO: 2341).

In some embodiments, a linker is a peptide linker. In some embodiments, a peptide linker comprises the formula of (Gly₄Ser)ₙ, wherein n=1-5 (SEQ ID NO: 1424). In some embodiments, n is 1. In some embodiments, n is 2. In some embodiments, n is 3. In some embodiments, n is 4. In some embodiments, n is 5. In some embodiments, a peptide linker is peptide linker is (Gly₄Ser)₃ (SEQ ID NO: 1342). In some embodiments, a peptide linker is peptide linker is (Gly₄Ser)₁ (SEQ ID NO: 1425).

In some embodiments, the linker links the C-terminus of a VH region to the N-terminus of a VL region of a polypeptide molecule. In some embodiments, the peptide linker links the C-terminus of a VL region to the N-terminus of a VH region of a polypeptide molecule. In some embodiments, the linker is a peptide linker.

In some embodiments, the binding domain comprises a VH region and a VL region linked by a peptide linker. Suitable linkers are generally those which allow both the VH region and the VL region to fold with a three-dimensional structure very similar to the VH region and the VL region produced individually without any linker or other component. In some embodiments, the C-terminus of the VH region of the binding domain is linked to the N-terminus of the VL region of the binding domain via the peptide linker. In some embodiments, the C-terminus of the VL region of the binding domain is linked to the N-terminus of the VH region of the binding domain via the peptide linker. In some embodiments, the binding domain is an scFv (that is, the peptide linker is an scFv linker). In some embodiments, the binding domain is a T cell receptor binding domain (e.g., a TCRβV-binding domain). In some embodiments, the binding domain is a tumor-associated antigen (TAA)-binding domain (e.g., a CD19 binding domain).

In some embodiments, the polypeptide molecule comprises a first binding domain and a second binding domain. In some embodiments, the first binding domain is linked (connected) to the second binding domain via a peptide linker. In some embodiments, a C-terminus of the first binding domain is linked to an N-terminus of the second binding domain via the peptide linker. In some embodiments, a C-terminus of the second binding domain is linked to a N-terminus of the first binding domain via the peptide linker. In some embodiments, the peptide linker comprises the formula of (Gly₄Ser)ₙ, wherein n=1-5 (SEQ ID NO: 1424). In some embodiments, the peptide linker is (Gly₄Ser)₃ (SEQ ID NO: 1342). In some embodiments, the peptide linker is (Gly₄Ser)₂ (SEQ ID NO: 2339). In some embodiments, the peptide linker is (Gly₄Ser)₁ (SEQ ID NO: 1425).

In some embodiments, the polypeptide molecule comprises a first binding domain and a second binding domain. In some embodiments, the first binding domain is linked (connected) to an Fc region via a peptide linker. In some embodiments, the second binding domain is linked (connected) to the Fc region via a peptide linker. In some embodiments, a C-terminus of the first binding domain is linked (connected) to the N-terminus of the Fc region via the peptide linker. In some embodiments, a C-terminus of the second binding domain is linked (connected) to the N-terminus of the Fc region via the peptide linker. In some embodiments, the peptide linker PLE comprises the amino acid sequence KTHTCP (SEQ ID NO: 2515). In some embodiments, the amino acid immediately before the lysine of the amino acid sequence KTHTCP (SEQ ID NO: 2515) is not an aspartate. In some embodiments, the peptide linker comprises a (Gly₄Ser)ₙ, n=1-5 sequence (SEQ ID NO: 1424) and the amino acid sequence KTHTCP (SEQ ID NO: 2515). In some embodiments, the peptide linker comprises or consists of the amino acid sequence of GGGGSGGGGSKTHTCP (SEQ ID NO: 2516). In some embodiments, the peptide linker comprises or consists of the amino acid sequence of DKTHTCP (SEQ ID NO: 2517). In some embodiments, the peptide linker comprises or consists of the amino acid sequence of DKTHTCPPCP (SEQ ID NO: 2518). In some embodiments, the peptide linker comprises or consists of the amino acid sequence of EPKSCDKTHTCPPCP (SEQ ID NO: 2519). In some embodiments, the peptide linker comprises or consists of the amino acid sequence of ERKCCVECPPCP (SEQ ID NO: 2520). In some embodiments, the peptide linker comprises or consists of the amino acid sequence of ELKTPLGDTTHTCPRCP (SEQ ID NO: 2521). In some embodiments, the peptide linker comprises or consists of the amino acid sequence of EPKSCDTPPPCPRCP (SEQ ID NO: 2522). In some embodiments, the peptide linker comprises or consists of the amino acid sequence of ESKYGPPCPSCP (SEQ ID NO: 2523).

In some embodiments, a polypeptide molecule comprises a homo-dimer or hetero-dimer (or other multimer) of any of the variable and constant domain configurations provided herein in non-covalent association with one another and/or with one or more monomeric VH or VL region (e.g., by disulfide bond(s)). In some embodiments, a VH region and a VL region of a polypeptide molecule of the disclosure can be linked by one or more engineered disulfide bond(s). In some embodiments, one cysteine that forms an engineered disulfide bond is located in a VH region, and another cysteine that forms an engineered disulfide bond is located in a VL region of the polypeptide molecule. In some embodiments, a position of the one or more engineered disulfide bond(s) is selected from the group consisting of VH44-VL100, VH100B-VL49, VH100-VL50, VH101-VL46, and VH105-VL43 according to EU numbering.

### Knob-In-Hole Configurations

In some embodiments, the polypeptide molecule comprises a “knobs-into-holes” configuration. Knobs-into-holes configurations are used to allow heavy chains to heterodimerize, and light chain mispairing is overcome by crossover of the antibody's entire Fab domain, variable domain, or constant domain within 1 Fab-arm of the polypeptide molecule. With this technology, bivalent (1:1), trivalent (2:1), or tetravalent (2:2) polypeptide molecules can be generated that retain the antigen-binding capacity of the parental polypeptide molecule while displaying variable avidity for the target epitope and distinct cytotoxic potential.

In some embodiments, the polypeptide molecule comprises an Fc region. In some embodiments, the Fc region of the polypeptide molecule is an Fc variant. In some embodiments, each polypeptide chain of the Fc variant has the same amino acid mutation(s). In some embodiments, different polypeptides of the Fc variant have different amino acid mutation(s) e.g., the knobs-into-holes format. Descriptions of the knobs-into-holes format can be found, for example, in WO 1996/027011, the content of which is incorporated by reference in its entirety.

In some embodiments, polypeptide molecule comprises an Fc region comprising a first polypeptide and a second polypeptide. In some embodiments, the first polypeptide of the Fc region comprises a hole mutation, and the second polypeptide of the Fc region comprises a knob mutation. In some embodiments, the first polypeptide of the Fc region comprises a hole mutation T366S, L368A, and/or Y407V according to EU numbering. In some embodiments, the first polypeptide of the Fc region comprises the hole mutations T366S, L368A, and Y407V according to EU numbering. In some embodiments, the second polypeptide of the Fc region comprises a knob mutation at the position T366 according to EU numbering. In some embodiments, the second polypeptide of the Fc region comprises the knob mutation T366W according to EU numbering.

In some embodiments, polypeptide molecule comprising the Fc region with the knobs-into-holes format further comprises an engineered disulfide bond. In some embodiments, the engineered disulfide bond is formed between an engineered cysteine on each of the polypeptides of the Fc region. In some embodiments, the first polypeptide of the Fc region comprising the hole mutation further comprises a mutation Y349C according to EU numbering, wherein the second polypeptide of the Fc region comprising the knob mutation further comprises a mutation S354C according to EU numbering, and wherein the Fc region comprises an engineered disulfide bond formed between these two cysteines.

Exemplary knob-in-hole configurations are provided at Table 7.

TABLE 7
Exemplary Knob-In-Hole Configurations

| | SEQ ID NO | |
|---|---|---|
| | Chain 1 | Chain 2 |
| Knob in hole Fc (LALAPG; -GK) | 1412 | 1414 |
| Knob in hole Fc (RF mutant) | 1413 | 1415 |

In some embodiments, the polypeptide molecule comprises an Fc region, and the Fc region has the knobs-into-holes format. In some embodiments, the first polypeptide of the Fc region comprises an amino acid sequence that is at least 80%, at least 85%, at least 90%, at least 91%, at least 92%, at least 93%, at least 94%, at least 95%, at least 96%, at least 97%, at least 98%, at least 99%, or 100%, identical to SEQ ID NO: 1412, and wherein the second polypeptide of the Fc region comprises an amino acid sequence that is at least 80%, at least 85%, at least 90%, at least 91%, at least 92%, at least 93%, at least 94%, at least 95%, at least 97%, at least 98%, at least 99%, or 100%, identical to SEQ ID NO: 1414.

In some embodiments, the polypeptide molecule comprises an Fc region, and the Fc region has the knobs-into-holes format. In some embodiments, the first polypeptide of the Fc region comprises an amino acid sequence that is at least 80%, at least 85%, at least 90%, at least 91%, at least 92%, at least 93%, at least 94%, at least 95%, at least 96%, at least 97%, at least 98%, at least 99%, or 100%, identical to SEQ ID NO: 1413, and wherein the second polypeptide of the Fc region comprises an amino acid sequence that is at least 80%, at least 85%, at least 90%, at least 91%, at least 92%, at least 93%, at least 94%, at least 95%, at least 96%, at least 97%, at least 98%, at least 99%, or 100%, identical to SEQ ID NO: 1415.

### Half-Life Extension Moieties

Polypeptide molecules of the disclosure can additionally be configured to comprise a half-life extension moiety. In some embodiments, the half-life extension moiety is connected to the polypeptide molecule via a linker. In some embodiments, the half-life extension moiety comprises a polypeptide. In some embodiments, the half-life extension moiety comprises a non-polypeptide moiety. In some embodiments, the half-life extension moiety comprises a polymer. In some embodiments, the half-life extension moiety is selected from the group consisting of a fragment crystallizable (Fc) region (e.g., a single chain Fc), an albumin (e.g., human serum albumin), a transferrin, a binding domain that specifically binds to transferrin or albumin (e.g., an albumin-binding domain (ABD)), an unstructured biodegradable proteins (XTEN), a polyethylene glycol (PEG), a glycine-rich homo-amino-acid polymer (HAP), a hydroxyethyl starch (HES), a polysialic acid (PSA), an immunoglobulin binding polypeptide, and a conformationally disordered polypeptide comprising proline, alanine and serine (PAS), or a derivative or functional fragment thereof.

In some embodiments, the half-life extension moiety comprises an immunoglobulin constant region or a portion thereof. In some embodiments, the half-life extension moiety is an antibody. In some embodiments, the half-life extension moiety is a human or humanized antibody. In some embodiments, the antibody is selected from the group consisting of 645gH1gL1, 645dsgH5gL4, 23-13-A01-sc02, A10m3 or a fragment thereof, DOM7r-31, DOM7 h-11-15, Alb-1, Alb-8, Alb-23, 10G, 10GE, and SA21. In some embodiments, the single domain antibody is 10G.

In some embodiments, the polypeptide molecule comprises a half-life extension moiety and the half-life extension moiety comprise an amino acid sequence that is at least 80%, at least 85%, at least 90%, at least 91%, at least 92%, at least 93%, at least 94%, at least 95%, at least 96%, at least 97%, at least 98%, at least 99%, or 100%, identical to EVQLVESGGGLVQPGNSLRLSCAASGFTFSKFGMSWVRQAPGKGLEWVSSISGSGRDTLY ADSVKGRFTISRDNAKTTLYLQMNSLRPEDTAVYYCTIGGSLSVSSQGTLVTVSS (SEQ ID NO: 2509).

In some embodiments, the polypeptide molecule comprises a half-life extension moiety and the half-life extension moiety comprise an amino acid sequence that is at least 80%, at least 85%, at least 90%, at least 91%, at least 92%, at least 93%, at least 94%, at least 95%, at least 96%, at least 97%, at least 98%, at least 99%, or 100%, identical to EVQLVESGGGLVQPGNSLRLSCAASGFTFSSFGMSWVRQAPGKGLEWVSSISGSGSDTLYA DSVKGRFTISRDNAKTTLYLQMNSLRPEDTAVYYCTIGGSLSRSSQGTLVTVSS (SEQ ID NO: 2510).

In some embodiments, the polypeptide molecule comprises a half-life extension moiety and the half-life extension moiety comprise an amino acid sequence that is at least 80%, at least 85%, at least 90%, at least 91%, at least 92%, at least 93%, at least 94%, at least 95%, at least 96%, at least 97%, at least 98%, at least 99%, or 100%, identical to EVQLVESGGGLVQPGGSLRLSCAASGSTFYTAVMGWVRQAPGKGLEWVAAIRWTALTTS YADSVKGRFTISRDGAKTTLYLQMNSLRPEDTAVYYCAARGTLGLFTTADSYDYWGQGT LVTVSS (SEQ ID NO: 2511).

In some embodiments, the polypeptide molecule comprises a half-life extension moiety, and the half-life extension moiety comprises a fragment crystallizable (Fc) region. In some embodiments, the Fc region is an Fc variant comprising one or more mutations that diminishes or removes an effector function. In some embodiments, the effector function is selected from the group consisting of antibody-dependent cellular cytotoxicity (ADCC), antibody-dependent cellular phagocytosis (ADCP), complement-dependent cytotoxicity (CDC), binding to a Fcγ receptor (FcγR), or any combination thereof. In some embodiments, the Fc region is an Fc variant lacking an endogenous glycan/glycosylation site. In some embodiments, one or both of the polypeptides of the Fc region comprise a mutation L234A, L235A, P329G, and/or N297G according to EU numbering. Further descriptions of such Fe variants can be found, for example, in US20120251531A1, the content of which is incorporated by reference in its entirety.

In some embodiments, the polypeptide molecule comprises an Fc region comprising two polypeptides. In some embodiments, one or both of the polypeptides of the Fc region comprise the mutation N297G according to EU numbering. In some embodiments, the N297G mutation removes an endogenous glycan/glycosylation site of the Fc region.

In some embodiments, one or both of the polypeptides of the Fc region comprise a mutation L234A, L235A, and/or P329G according to EU numbering. In some embodiments, one or both of the polypeptides of the Fc region comprise mutations L234A and L235A according to EU numbering. In some embodiments, one or both of the polypeptides of the Fc region comprise mutations L234A, L235A, and P329G according to EU numbering. In some embodiments, both of the polypeptides of the Fc region comprise mutations L234A, L235A, and P329G according to EU numbering. In some embodiments, one or both of the polypeptides of the Fc region further comprise a mutation N297G according to EU numbering. In some embodiments, both of the polypeptides of the Fc region further comprise a mutation N297G according to EU numbering. In some embodiments, the Fc region lacks one or more effector functions and/or lacks glycosylation.

In some embodiments, one or both of the polypeptides of the Fc region comprise the mutation H435R and/or Y436F according to EU numbering. In some embodiments, one or both of the polypeptides of the Fc region comprise the mutations H435R/Y436F according to EU numbering. In some embodiments, only the first polypeptide of the Fc region comprises the hole mutation(s) and further comprises the mutations H435R/Y436F according to EU numbering. In some embodiments, the H435R and/or Y436F mutations in the CH3 domain diminishes or removes protein A binding capability of the Fc region.

In some embodiments, one or both of the polypeptides of the Fc region comprise the mutation the deletion of G446 and/or K447 according to EU numbering. In some embodiments, both of the polypeptides of the Fc region comprise the mutation corresponding to the deletion of G446 and K447 according to EU numbering.

Half-life extension moieties can be configured into any of the polypeptide molecules of the disclosure. In some embodiments, the Fc variant comprises the mutations L234A, L235A, P329G, N297G, and deletion of G446 and K447 according to EU numbering. In some embodiments, the Fc variant comprises the mutations L234A, L235A, P329G, H435R, Y436F according to EU numbering. In some embodiments, the Fc variant has a difference in at least one effector function imparted by an Fc region comprising said wild-type Fc domain (e.g., an improvement or reduction in the ability of the Fc region to bind to Fc receptors (e.g. FcγRI, FcγRII, or FcγRIII) or complement proteins (e.g. C1q), or to trigger antibody-dependent cytotoxicity (ADCC), phagocytosis, or complement-dependent cytotoxicity (CDCC)). In some embodiments, the Fc variant provides an engineered cysteine residue.

In some embodiments, the Fc region of the disclosure is an Fc variant known to impart a difference (e.g., an enhancement or reduction) in effector function and/or FcR binding. Specifically, the Fc region may include, for example, a modification (e.g., a substitution) at one or more of the amino acid positions disclosed in International PCT Publications WO88/07089A1, WO96/14339A1, WO98/05787A1, WO98/23289A1, WO99/51642A1, WO99/58572A1, WO00/09560A2, WO00/32767A1, WO00/42072A2, WO02/44215A2, WO02/060919A2, WO03/074569A2, WO04/016750A2, WO04/029207A2, WO04/035752A2, WO04/063351A2, WO04/074455A2, WO04/099249A2, WO05/040217A2, WO04/044859, WO05/070963A1, WO05/077981A2, WO05/092925A2, WO05/123780A2, WO06/019447A1, WO06/047350A2, and WO06/085967A2; US Patent Publication Nos. US2007/0231329, US2007/0231329, US2007/0237765, US2007/0237766, US2007/0237767, US2007/0243188, US20070248603, US20070286859, US20080057056; or U.S. Pat. Nos. 5,648,260; 5,739,277; 5,834,250; 5,869,046; 6,096,871; 6,121,022; 6,194,551; 6,242,195; 6,277,375; 6,528,624; 6,538,124; 6,737,056; 6,821,505; 6,998,253; 7,083,784; 7,404,956, and 7,317,091, the content of each of which is incorporated by reference in its entirety. In some embodiments, the specific modification (e.g., the specific substitution of one or more amino acids disclosed in the art) is made at one or more of the disclosed amino acid positions. In another embodiment, a different modification at one or more of the disclosed amino acid positions (e.g., a different substitution of one or more amino acid positions disclosed in the art) is made.

The Fc region of IgG can be modified according to well recognized procedures such as site directed mutagenesis and the like to yield modified IgG or Fc fragments or portions thereof that will be bound by Fc receptors. Such modifications include modifications remote from the Fc receptor contact sites as well as modifications within the contact sites that preserve or even enhance binding to the Fc receptors. For example, the following single amino acid residues in human IgG1 Fc (Fc 71) can be substituted without significant loss of Fc binding affinity for Fc receptors: P238A, S239A, K246A, K248A, D249A, M252A, T256A, E258A, T260A, D265A, S267A, H268A, E269A, D270A, E272A, L274A, N276A, Y278A, D280A, V282A, E283A, H285A, N286A, T289A, K290A, R292A, E293A, E294A, Q295A, Y296F, N297A, S298A, Y300F, R301A, V303A, V305A, T307A, L309A, Q311A, D312A, N315A, K317A, E318A, K320A, K322A, S324A, K326A, A327Q, P329A, A330Q, P331A, E333A, K334A, T335A, S337A, K338A, K340A, Q342A, R344A, E345A, Q347A, R355A, E356A, M358A, T359A, K360A, N361A, Q362A, Y373A, S375A, D376A, A378Q, E380A, E382A, S383A, N384A, Q386A, E388A, N389A, N390A, Y391F, K392A, L398A, S400A, D401A, D413A, K414A, R416A, Q418A, Q419A, N421A, V422A, S424A, E430A, N434A, T437A, Q438A, K439A, S440A, S444A, and K447A, whereas, for example, P238A represents wild type proline substituted by alanine at position number 238. As an example, a specific embodiment incorporates the N297A mutation, removing a highly conserved N-glycosylation site. In addition to alanine, other amino acids may be substituted for the wild type amino acids at the positions specified above. Mutations may be introduced into Fc giving rise to more than one hundred Fc regions distinct from the native Fc. Additionally, combinations of two, three, or more of these individual mutations may be introduced together, giving rise to hundreds more Fc regions. Moreover, one polypeptide chain of the Fc region may be mutated and the other polypeptide chain of the Fc region not mutated at all, or they both may be mutated but with different mutations.

In some embodiments, the above-mentioned Fc mutations confer a new functionality upon the Fc region. For example, the substitution N297A removes a highly conserved N-glycosylation site. The effect of this mutation is to reduce immunogenicity, thereby enhancing circulating half-life of the Fc region, and to render the Fc region incapable of binding to FcγRI, FcγRIIA, FcγRIIB, and FcγRIIIA, without compromising affinity (Routledge et al. 1995, Transplantation 60:847; Friend et al. 1999, Transplantation 68:1632; Shields et al. 1995, J. Biol. Chem. 276:6591). As a further example of new functionality, affinity for Fc receptors may be increased beyond that of wild type. This increased affinity may reflect an increased “on” rate, a decreased “off” rate or both an increased “on” rate and a decreased “off” rate. Examples of mutations believed to impart an increased affinity for Fc receptors include, but are not limited to, T256A, T307A, E380A, and N434A (Shields et al. 2001, J. Biol. Chem. 276:6591).

Additionally, at least three human Fc gamma receptors appear to recognize a binding site on IgG within the lower hinge region, generally amino acids 234-237. Therefore, another example of new functionality and potential decreased immunogenicity may arise from mutations of this region, as for example by replacing amino acids 233-236 of human IgG1 “ELLG” to the corresponding sequence from IgG2 “PVA” (with one amino acid deletion). It has been shown that FcγRI, FcγRII, and FcγRIII, which mediate various effector functions will not bind to IgG1 when such mutations have been introduced. Ward and Ghetie 1995, Therapeutic Immunology 2:77 and Armour et al. 1999, Eur. J. Immunol. 29:2613.

In some embodiments, the Fc region is hemi-glycosylated (for example, containing a first, glycosylated CH2 region and a second, aglycosylated CH2 region. In some embodiments, the Fc region is fully glycosylated. In other embodiments, the Fc region is aglycosylated.

In some embodiments, the Fc region comprises an amino acid substitution that alters the antigen-independent effector functions of the Ig constant region, in particular the circulating half-life of the protein. Such proteins exhibit either increased or decreased binding to an Fc receptor when compared to proteins lacking these substitutions and, therefore, have an increased or decreased half-life in serum, respectively. Fc variants with improved affinity for an Fc receptor are anticipated to have longer serum half-lives, and such molecules have useful applications in methods of treating mammals where long half-life of the administered polypeptide is desired, e.g., to treat a chronic disease or disorder (see, e.g., U.S. Pat. Nos. 7,348,004, 7,404,956, and 7,862,820). In contrast, Fc variants with decreased Fc receptor binding affinity are expected to have shorter half-lives, and such molecules are also useful, for example, for administration to a mammal where a shortened circulation time may be advantageous, e.g., for in vivo diagnostic imaging or in situations where the starting polypeptide has toxic side effects when present in the circulation for prolonged periods. Fc variants with decreased Fc receptor binding affinity are also less likely to cross the placenta and, thus, are also useful in the treatment of diseases or disorders in pregnant women. In addition, other applications in which reduced Fc receptor binding affinity may be desired include those applications in which localization the brain, kidney, and/or liver is desired. In one exemplary embodiment, the polypeptide molecule of the disclosure exhibits reduced transport across the epithelium of kidney glomeruli from the vasculature. In another embodiment, the polypeptide molecule of the disclosure exhibits reduced transport across the blood brain barrier (BBB) from the brain, into the vascular space. In some embodiments, the Fc region comprises one or more amino acid substitutions within the “Fc receptor binding loop” of an Ig constant region. The Fc receptor binding loop is comprised of amino acid residues 280-299 (according to EU numbering) of a wild-type, full-length, Fc region. In other embodiments, the Fc region comprises one or more amino acid substitutions at an amino acid position corresponding to any one of the following EU positions: 256, 277-281, 283-288, 303-309, 313, 338, 342, 376, 381, 384, 385, 387, 434 (e.g., N434A or N434K), and 438, which results in altered Fc receptor binding affinity. Exemplary amino acid substitutions which altered Fc receptor binding activity are disclosed in International PCT Publication No. WO05/047327.

In some embodiments, the half-life extension moiety of the polypeptide molecule comprises a single chain Fc (scFc). In some embodiments, the scFc comprises a first polypeptide and a second polypeptide each of which comprises a hinge region, a CH2 domain, and a CH3 domain, and wherein the Fc polypeptide sequences are joined by a polypeptide linker so as to allow the formation of a functional Fc region from a single polypeptide having the amino to carboxyl order: Hinge-CH2-CH3-linker-Hinge-CH2-CH3. Further descriptions of scFc can be found, for example, in US20110081345, the content of which is incorporated by reference in its entirety.

In some embodiments, the half-life extension moiety of the polypeptide molecule comprises a serum protein or a fragment thereof. In some embodiments, the serum protein comprises a thyroxine-binding protein, a transthyretin, a 1-acid glycoprotein, a transferrin, transferrin receptor or a transferrin-binding portion thereof, a fibrinogen, or an albumin.

In some embodiments, the half-life extension moiety of the polypeptide molecule comprises an albumin or a functional fragment thereof. Human serum albumin (I, or HA), a protein of about 609 amino acids in its full-length form, is responsible for a significant proportion of the osmotic pressure of serum and also functions as a carrier of endogenous and exogenous ligands. The term “albumin” as used herein includes full-length albumin or a functional fragment, variant, derivative, or analog thereof. In some embodiments, the albumin or a fragment or variant thereof extends (or is capable of extending) the half-life of the polypeptide. Further examples of albumin or the fragments or variants thereof are disclosed in US Pat. Publ. Nos. 2008/0194481 A1, 2008/0004206 A1, 2008/0161243 A1, 2008/0261877 A1, or 2008/0153751 A1 or PCT Appl. Publ. Nos. 2008/033413 A2, 2009/058322 A1, or 2007/021494 A2, the content of each of which is incorporated by reference in its entirety.

In some embodiments, the half-life extension moiety of the polypeptide molecule comprises a transferrin or a fragment thereof. Any transferrin may be used to make the polypeptide molecules of the disclosure. As an example, wild-type human Tf (Tf) is a 679 amino acid protein, of approximately 75 Kda (not accounting for glycosylation), with two main domains, N (about 330 amino acids) and C (about 340 amino acids), which appear to originate from a gene duplication. See GenBank accession numbers NM001063, XM002793, M12530, XM039845, XM 039847 and S95936. Transferrin comprises two domains, N domain and C domain. N domain comprises two subdomains, N1 domain and N2 domain, and C domain comprises two subdomains, C1 domain and C2 domain. In some embodiments, the transferrin portion of the polypeptide molecule includes a transferrin splice variant. In one example, a transferrin splice variant can be a splice variant of human transferrin, e.g., Genbank Accession AAA61140. In some embodiments, the transferrin portion of the polypeptide molecule includes one or more domains of the transferrin sequence, e.g., N domain, C domain, N1 domain, N2 domain, C1 domain, C2 domain or any combinations thereof.

In some embodiments, the half-life extension moiety of the polypeptide molecule comprises a binding moiety that binds serum protein or a fragment thereof. In some embodiments, the binding moiety is a polypeptide, a ligand, or a small molecule. In some embodiments, the serum protein comprises a thyroxine-binding protein, a transthyretin, a 1-acid glycoprotein, a transferrin, transferrin receptor or a transferrin-binding portion thereof, a fibrinogen, or an albumin. In some embodiments, the half-life extension moiety comprises an albumin binding moiety, which comprises an albumin binding peptide, a bacterial albumin binding domain, an albumin-binding antibody fragment, or any combinations thereof. In some embodiments, the albumin binding moiety is an antibody. In some embodiments, the antibody comprises a single domain antibody, a single chain variable fragment, or a Fab. In some embodiments, the single domain antibody comprises a single domain antibody that binds to albumin. In some embodiments, the antibody is a human or humanized antibody. In some embodiments, the antibody is selected from the group consisting of 645gH1gL1, 645dsgH5gL4, 23-13-A01-sc02. In some embodiments, the albumin binding moiety is a bacterial albumin binding protein, an antibody or an antibody fragment thereof including domain antibodies (see U.S. Pat. No. 6,696,245), or one of streptococcal protein G (Konig, T. and Skerra, A. (1998) J. Immunol. Methods 218, 73-83). Other examples of albumin binding peptides that can be used as conjugation partner are, for instance, those having a Cys-Xaa₁-Xaa₂-Xaa₃-Xaa₄-Cys consensus sequence, wherein Xaa₁ is Asp, Asn, Ser, Thr, or Trp; Xaa₂ is Asn, Gln, H is, Ile, Leu, or Lys; Xaa₃ is Ala, Asp, Phe, Trp, or Tyr; and Xaa₄ is Asp, Gly, Leu, Phe, Ser, or Thr as described in US patent application 2003/0069395 or Dennis et al. (Dennis et al. (2002) J. Biol. Chem. 277, 35035-35043), the content of each of which is incorporated by reference in its entirety.

In some embodiments, the half-life extension moiety of the polypeptide molecule comprises a PAS sequence. A PAS sequence, as refers to an amino acid sequence comprising mainly alanine and serine residues or comprising mainly alanine, serine, and proline residues, the amino acid sequence forming random coil conformation under physiological conditions. Accordingly, the PAS sequence is a building block, an amino acid polymer, or a sequence cassette comprising, consisting essentially of, or consisting of alanine, serine, and proline which can be used as a part of the heterologous moiety in the polypeptide molecule. Yet, the skilled person is aware that an amino acid polymer also may form random coil conformation when residues other than alanine, serine, and proline are added as a minor constituent in the PAS sequence. The term “minor constituent” as used herein means that amino acids other than alanine, serine, and proline may be added in the PAS sequence to a certain degree, e.g., up to about 12%, i.e., about 12 of 100 amino acids of the PAS sequence, up to about 10%, i.e. about 10 of 100 amino acids of the PAS sequence, up to about 9%, i.e., about 9 of 100 amino acids, up to about 8%, i.e., about 8 of 100 amino acids, about 6%, i.e., about 6 of 100 amino acids, about 5%, i.e., about 5 of 100 amino acids, about 4%, i.e., about 4 of 100 amino acids, about 3%, i.e., about 3 of 100 amino acids, about 2%, i.e., about 2 of 100 amino acids, about 1%, i.e., about 1 of 100 of the amino acids. The amino acids different from alanine, serine and proline may be selected from the group consisting of Arg, Asn, Asp, Cys, Gln, Glu, Gly, His, Ile, Leu, Lys, Met, Phe, Thr, Trp, Tyr, and Val. Under physiological conditions, the PAS sequence stretch forms a random coil conformation and thereby can mediate an increased in vivo and/or in vitro stability to the polypeptide. In some embodiments, the PAS sequences that form random coil domain are biologically inert, especially with respect to proteolysis in blood plasma, immunogenicity, isoelectric point/electrostatic behavior, binding to cell surface receptors or internalization, but are still biodegradable. Non-limiting examples of the PAS sequences forming random coil conformation comprise an amino acid sequence selected from the group consisting of ASPAAPAPASPAAPAPSAPA (SEQ ID NO: 2393), AAPASPAPAAPSAPAPAAPS (SEQ ID NO: 2394), APSSPSPSAPSSPSPASPSS (SEQ ID NO: 2395), APSSPSPSAPSSPSPASPS (SEQ ID NO: 2396), SSPSAPSPSSPASPSPSSPA (SEQ ID NO: 2397), AASPAAPSAPPAAASPAAPSAPPA (SEQ ID NO: 2398) and ASAAAPAAASAAASAPSAAA (SEQ ID NO: 2399) or any combinations thereof. Additional examples of PAS sequences are known from, e.g., US Pat. Publ. No. 2010/0292130 A1 and PCT Appl. Publ. No. WO 2008/155134 A1, the content of each of which is incorporated by reference in its entirety.

In some embodiments, the half-life extension moiety of the polypeptide molecule comprises a glycine-rich homo-amino-acid polymer (HAP). The HAP sequence can comprise a repetitive sequence of glycine, which has at least 50 amino acids, 100 amino acids, 120 amino acids, 140 amino acids, 160 amino acids, 180 amino acids, 200 amino acids, 250 amino acids, 300 amino acids, 350 amino acids, 400 amino acids, 450 amino acids, or 500 amino acids in length. In some embodiments, the HAP sequence is capable of extending half-life of a moiety fused to or linked to the HAP sequence. Non-limiting examples of the HAP sequence includes, but are not limited to (Gly)n (SEQ ID NO: 2400), (Gly₄Ser)ₙ (SEQ ID NO: 2401) or S(Gly₄Ser)ₙ (SEQ ID NO: 2402), wherein n is 1, 2, 3, 4, 5, 6, 7, 8, 9, 10, 11, 12, 13, 14, 15, 16, 17, 18, 19, or 20. In one embodiment, n is about 20, 21, 22, 23, 24, 25, 26, 26, 28, 29, 30, 31, 32, 33, 34, 35, 36, 37, 38, 39, or 40. In another embodiment, n is about 50, 60, 70, 80, 90, 100, 110, 120, 130, 140, 150, 160, 170, 180, 190, or 200.

In some embodiments, the half-life extension moiety of the polypeptide molecule comprises a polyethylene glycol, ethylene glycol/propylene glycol copolymer, carboxymethylcellulose, dextran, polyvinyl alcohol, poly(alkylene oxide), poly(vinyl pyrrolidone), poly(vinyl alcohol), polyoxazoline, or poly(acryloylmorpholine). Also provided by the disclosure are chemically modified derivatives of the polypeptide molecules of the disclosure which may provide additional advantages such as increased solubility, stability and circulating time of the polypeptide, or decreased immunogenicity (see U.S. Pat. No. 4,179,337). The chemical moieties for modification can be selected from the group consisting of water soluble polymers including, but not limited to, polyethylene glycol, ethylene glycol/propylene glycol copolymers, carboxymethylcellulose, dextran, and polyvinyl alcohol. The polypeptide molecule may be modified at random positions within the molecule or at the N- or C-terminus, or at predetermined positions within the molecule and may include one, two, three or more attached chemical moieties. The polymer can be of any molecular weight and can be branched or unbranched. For polyethylene glycol, In some embodiments, the molecular weight is between about 1 kDa and about 100 kDa for ease in handling and manufacturing. Other sizes may be used, depending on the desired profile (e.g., the duration of sustained release desired, the effects, if any on biological activity, the ease in handling, the degree or lack of antigenicity and other known effects of the polyethylene glycol to a protein or analog). For example, the polyethylene glycol may have an average molecular weight of about 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 25,000, 30,000, 35,000, 40,000, 45,000, 50,000, 55,000, 60,000, 65,000, 70,000, 75,000, 80,000, 85,000, 90,000, 95,000, or 100,000 kDa. In some embodiments, the polyethylene glycol may have a branched structure. Branched polyethylene glycols are described, for example, in U.S. Pat. No. 5,643,575; Morpurgo et al., Appl. Biochem. Biotechnol. 56:59-72 (1996); Vorobjev et al., Nucleosides Nucleotides 18:2745-2750 (1999); and Caliceti et al., Bioconjug. Chem. 10:638-646 (1999). The number of polyethylene glycol moieties attached to each polypeptide molecule of the disclosure (i.e., the degree of substitution) may also vary. For example, the pegylated proteins of the disclosure may be linked, on average, to 1, 2, 3, 4, 5, 6, 7, 8, 9, 10, 12, 15, 17, 20, or more polyethylene glycol molecules. Similarly, the average degree of substitution within ranges such as 1-3, 2-4, 3-5, 4-6, 5-7, 6-8, 7-9, 8-10, 9-11, 10-12, 11-13, 12-14, 13-15, 14-16, 15-17, 16-18, 17-19, or 18-20 polyethylene glycol moieties per protein molecule. Methods for determining the degree of substitution are discussed, for example, in Delgado et al., Crit. Rev. Thera. Drug Carrier Sys. 9:249-304 (1992).

In some embodiments, the half-life extension moiety of the polypeptide molecule comprises a hydroxyethyl starch (HES) or a derivative thereof. Hydroxyethyl starch (HES) is a derivative of naturally occurring amylopectin and is degraded by alpha-amylase in the body. HES is a substituted derivative of the carbohydrate polymer amylopectin, which is present in corn starch at a concentration of up to 95% by weight. HES exhibits advantageous biological properties and is used as a blood volume replacement agent and in hemodilution therapy in the clinics (Sommermeyer et al., Krankenhauspharmazie, 8(8), 271-278 (1987); and Weidler et al., Arzneim.-Forschung/Drug Res., 41, 494-498 (1991)). Amylopectin contains glucose moieties, wherein in the main chain alpha-1,4-glycosidic bonds are present and at the branching sites alpha-1,6-glycosidic bonds are found. The physical-chemical properties of this molecule are mainly determined by the type of glycosidic bonds. Due to the nicked alpha-1,4-glycosidic bond, helical structures with about six glucose-monomers per turn are produced. The physico-chemical as well as the biochemical properties of the polymer can be modified via substitution. The introduction of a hydroxyethyl group can be achieved via alkaline hydroxyethylation. By adapting the reaction conditions it is possible to exploit the different reactivity of the respective hydroxy group in the unsubstituted glucose monomer with respect to a hydroxyethylation. Owing to this fact, the skilled person is able to influence the substitution pattern to a limited extent. HES is mainly characterized by the molecular weight distribution and the degree of substitution. The degree of substitution, denoted as DS, relates to the molar substitution, is known to the skilled people. See Sommermeyer et al., Krankenhauspharmazie, 8(8), 271-278 (1987), as cited above, in particular p. 273. In one embodiment, hydroxyethyl starch has a mean molecular weight (weight mean) of from 1 to 300 kD, from 2 to 200 kD, from 3 to 100 kD, or from 4 to 70 kD. Hydroxyethyl starch can further exhibit a molar degree of substitution of from 0.1 to 3, preferably 0.1 to 2, more preferred, 0.1 to 0.9, preferably 0.1 to 0.8, and a ratio between C2:C6 substitution in the range of from 2 to 20 with respect to the hydroxyethyl groups. A non-limiting example of HES having a mean molecular weight of about 130 kD is a HES with a degree of substitution of 0.2 to 0.8 such as 0.2, 0.3, 0.4, 0.5, 0.6, 0.7, or 0.8, preferably of 0.4 to 0.7 such as 0.4, 0.5, 0.6, or 0.7. In a specific embodiment, HES with a mean molecular weight of about 130 kD is VOLUVEN® from Fresenius. VOLUVEN® is an artificial colloid, employed, e.g., for volume replacement used in the therapeutic indication for therapy and prophylaxis of hypovolemia. The characteristics of VOLUVEN® are a mean molecular weight of 130,000+/−20,000 D, a molar substitution of 0.4 and a C2:C6 ratio of about 9:1. In other embodiments, ranges of the mean molecular weight of hydroxyethyl starch are, e.g., 4 to 70 kD or 10 to 70 kD or 12 to 70 kD or 18 to 70 kD or 50 to 70 kD or 4 to 50 kD or 10 to 50 kD or 12 to 50 kD or 18 to 50 kD or 4 to 18 kD or 10 to 18 kD or 12 to 18 kD or 4 to 12 kD or 10 to 12 kD or 4 to 10 kD. In still other embodiments, the mean molecular weight of hydroxyethyl starch employed is in the range of from more than 4 kD and below 70 kD, such as about 10 kD, or in the range of from 9 to 10 kD or from 10 to 11 kD or from 9 to 11 kD, or about 12 kD, or in the range of from 11 to 12 kD) or from 12 to 13 kD or from 11 to 13 kD, or about 18 kD, or in the range of from 17 to 18 kD or from 18 to 19 kD or from 17 to 19 kD, or about 30 kD, or in the range of from 29 to 30, or from 30 to 31 kD, or about 50 kD, or in the range of from 49 to 50 kD or from 50 to 51 kD or from 49 to 51 kD. In certain embodiments, the heterologous moiety can be mixtures of hydroxyethyl starches having different mean molecular weights and/or different degrees of substitution and/or different ratios of C2: C6 substitution. Therefore, mixtures of hydroxyethyl starches may be employed having different mean molecular weights and different degrees of substitution and different ratios of C2: C6 substitution, or having different mean molecular weights and different degrees of substitution and the same or about the same ratio of C2:C6 substitution, or having different mean molecular weights and the same or about the same degree of substitution and different ratios of C2:C6 substitution, or having the same or about the same mean molecular weight and different degrees of substitution and different ratios of C2:C6 substitution, or having different mean molecular weights and the same or about the same degree of substitution and the same or about the same ratio of C2:C6 substitution, or having the same or about the same mean molecular weights and different degrees of substitution and the same or about the same ratio of C2:C6 substitution, or having the same or about the same mean molecular weight and the same or about the same degree of substitution and different ratios of C2: C6 substitution, or having about the same mean molecular weight and about the same degree of substitution and about the same ratio of C2:C6 substitution.

In some embodiments, the half-life extension moiety of the polypeptide molecule comprises a polysialic acid (PSA) or a derivative thereof. Polysialic acids (PSAs) are naturally occurring unbranched polymers of sialic acid produced by certain bacterial strains and in mammals in certain cells Roth J., et al. (1993) in Polysialic Acid: From Microbes to Man, eds Roth J., Rutishauser U., Troy F. A. (Birkhauser Verlag, Basel, Switzerland), pp 335-348. They can be produced in various degrees of polymerisation (e.g., from n=about 80 or more sialic acid residues down to n=2) by limited acid hydrolysis or by digestion with neuraminidases, or by fractionation of the natural, bacterially derived forms of the polymer. The composition of different polysialic acids also varies such that there are homopolymeric forms i.e., the alpha-2,8-linked polysialic acid comprising the capsular polysaccharide of E. coli strain K1 and the group-B meningococci, which is also found on the embryonic form of the neuronal cell adhesion molecule (N-CAM). Heteropolymeric forms also exist—such as the alternating alpha-2,8 alpha-2,9 polysialic acid of E. coli strain K92 and group C polysaccharides of N. meningitidis. Sialic acid may also be found in alternating copolymers with monomers other than sialic acid such as group W135 or group Y of N. meningitidis. Polysialic acids have important biological functions including the evasion of the immune and complement systems by pathogenic bacteria and the regulation of glial adhesiveness of immature neurons during fetal development (wherein the polymer has an anti-adhesive function) Cho and Troy, P.N.A.S., USA, 91 (1994) 11427-11431, which is incorporated by reference herein in its entirety. The alpha-2,8-linked polysialic acid of E. coli strain K1 is also known as ‘colominic acid’ and is used (in various lengths) to exemplify the present disclosure. Various methods of attaching or conjugating polysialic acids to a polypeptide have been described, for example, in U.S. Pat. No. 5,846,951 and US 2007/0191597 A1.

In some embodiments, the half-life extension moiety of the polypeptide molecule comprises an XTEN Sequence. As used here “XTEN sequence” refers to extended length polypeptides with non-naturally occurring, substantially non-repetitive sequences that are composed mainly of small hydrophilic amino acids, with the sequence having a low degree or no secondary or tertiary structure under physiologic conditions. As a fusion protein partner, XTENs can serve as a carrier, conferring certain desirable pharmacokinetic, physicochemical and pharmaceutical properties when linked to the binding domains of the disclosure. Such desirable properties include but are not limited to enhanced pharmacokinetic parameters and solubility characteristics. In some embodiments, the XTEN sequence of the disclosure is a polypeptide having greater than about 20, 30, 40, 50, 60, 70, 80, 90, 100, 150, 200, 250, 300, 350, 400, 450, 500, 550, 600, 650, 700, 750, 800, 850, 900, 950, 1000, 1200, 1400, 1600, 1800, or 2000 amino acid residues. In some embodiments, XTEN is a polypeptide having greater than about 20 to about 3000 amino acid residues, greater than 30 to about 2500 residues, greater than 40 to about 2000 residues, greater than 50 to about 1500 residues, greater than 60 to about 1000 residues, greater than 70 to about 900 residues, greater than 80 to about 800 residues, greater than 90 to about 700 residues, greater than 100 to about 600 residues, greater than 110 to about 500 residues, or greater than 120 to about 400 residues. In some embodiments, the XTEN sequence of the disclosure comprises one or more sequence motif of 9 to 14 amino acid residues or an amino acid sequence at least 80%, 90%, 91%, 92%, 93%, 94%, 95%, 96%, 97%, 98%, or 99% identical to the sequence motif, wherein the motif comprises, consists essentially of, or consists of 4 to 6 types of amino acids selected from the group consisting of glycine (G), alanine (A), serine (S), threonine (T), glutamate I and proline (P). In some embodiments, an XTEN sequence comprises multiple units of non-overlapping sequence motifs of the AD motif family, or of the AE motif family, or of the AF motif family, or of the AG motif family, or of the AM motif family, or of the AQ motif family, or of the BC family, or of the BD family, with the resulting XTEN exhibiting the range of homology. In other embodiments, the XTEN comprises multiple units of motif sequences from two or more of the motif families. These sequences can be selected to achieve desired physical/chemical characteristics, including such properties as net charge, hydrophilicity, lack of secondary structure, or lack of repetitiveness that are conferred by the amino acid composition of the motifs, described more fully below. In other embodiments, the motifs incorporated into the XTEN can be selected and assembled using the methods described herein to achieve an XTEN of about 36 to about 3000 amino acid residues. In some embodiments, the XTEN sequence affects the physical or chemical property, e.g., pharmacokinetics, of the polypeptide molecule of the present disclosure. In some embodiments, the XTEN sequence exhibits one or more of the following advantageous properties: conformational flexibility, enhanced aqueous solubility, high degree of protease resistance, low immunogenicity, low binding to mammalian receptors, or increased hydrodynamic (or Stokes) radii. In some embodiments, the XTEN sequence linked to the binding domain(s) of the disclosure increases pharmacokinetic properties such as longer terminal half-life or increased area under the curve (AUC), so that the polypeptide molecule stays in vivo for an increased period of time compared to a control polypeptide molecule lacking the XTEN sequence. In some embodiments, the XTEN sequence increases pharmacokinetic properties such as longer terminal half-life or increased area under the curve (AUC), so that the polypeptide molecule stays in vivo for an increased period of time compared to a control polypeptide molecule lacking the XTEN sequence. A variety of methods and assays can be employed to determine the physical/chemical properties of proteins comprising the XTEN sequence. Such methods include, but are not limited to analytical centrifugation, EPR, HPLC-ion exchange, HPLC-size exclusion, HPLC-reverse phase, light scattering, capillary electrophoresis, circular dichroism, differential scanning calorimetry, fluorescence, HPLC-ion exchange, HPLC-size exclusion, IR, NMR, Raman spectroscopy, refractometry, and UV/Visible spectroscopy. Additional methods are disclosed in Amau et al., Prot Expr and Purif 48, 1-13 (2006). More description of XTEN can be found, for example, in US Patent Publication Nos. 2010/0239554 A1, 2010/0323956 A1, 2011/0046060 A1, 2011/0046061 A1, 2011/0077199 A1, or 2011/0172146 A1, or International Patent Publication Nos. WO 2010091122 A1, WO 2010144502 A2, WO 2010144508 A1, WO 2011028228 A1, WO 2011028229 A1, or WO 2011028344 A2, the content of each of which is incorporated herein by its entirety.

In some embodiments, the half-life extension moiety of the polypeptide molecule comprises an immunoglobulin-binding polypeptide. In some embodiments, the binding polypeptide is a polypeptide, a ligand, or a small molecule. In some embodiments, the immunoglobulin-binding polypeptide comprises IgG1, IgG2, IgG3, IgG4, slgA, IgM or IgD. In some embodiments, the immunoglobulin-binding polypeptide binds to an Fc region and improves the half-life of the polypeptide molecule. In some embodiments, the immunoglobulin-binding polypeptide has greater than about 5, 6, 7, 8, 9, 10, 11, 12, 13, 14, 15, 16, 17, 18, 19, 20, 30, 40, 50, 60, 70, 80, 90, 100, 150, 200, 250, 300, 350, 400, 450, or 500 amino acid residues, including any range or subrange therebetween. In some embodiments, the immunoglobulin-binding polypeptide comprises a 13-mer IgG-Fc domain binding peptide (IgGBP). Further description of the immunoglobulin-binding polypeptide useful for the present disclosure can be found, for example, in DeLano W L, et al. (2000) Science 287: 1279-1283; U.S. Patent Publication No. 2017/0334954; or U.S. Patent Publication No. 2017/0210777, the content of each of which is incorporated herein by its entirety.

### Polynucleotides and Vectors

In some embodiments, the present disclosure provides recombinant nucleic acid molecules (i.e., polynucleotides) encoding the polypeptide molecules described herein. Persons having skill in the art will be familiar with method of encoding polypeptides within polynucleotides.

In some embodiments, the present disclosure provides a recombinant nucleic acid molecule encoding a polypeptide molecule, wherein the polypeptide molecule comprises a T cell-binding domain (e.g., TCRβ-binding domain) and/or a TAA-binding domain.

In some embodiments, the present disclosure provides a recombinant nucleic acid molecule encoding a polypeptide molecule, wherein the polypeptide molecule comprises at least one binding domain. In some embodiments, the present disclosure provides a recombinant nucleic acid molecule encoding a polypeptide molecule, wherein the polypeptide molecule comprises a first binding domain and a second binding domain. In some embodiments, the first binding domain is a TCRβV-binding domain (e.g., a TCRβV6-5-binding domain or a TCRβV12-3-binding domain). In some embodiments, the second binding domain is a tumor-associated antigen binding domain (e.g., CD19-binding domain).

The present disclosure further relates to variants of polynucleotides disclosed herein. The polynucleotide variants can contain alterations in the coding regions, non-coding regions, or both. In some embodiments, a polynucleotide variant is produced to modulate or alter expression (or expression levels) of the encoded polypeptide molecule. In some embodiments, a polynucleotide variant is produced to increase expression of the encoded polypeptide molecule. In some embodiments, a polynucleotide variant is produced to decrease expression of the encoded polypeptide molecule. In some embodiments, a polynucleotide variant has increased expression of the encoded polypeptide molecule as compared to a parental polynucleotide sequence. In some embodiments, a polynucleotide variant has decreased expression of the encoded polypeptide molecule as compared to a parental polynucleotide sequence.

In some embodiments, a polynucleotide variant comprising a nucleotide sequence that is at least about 75%, about 80%, about 85%, about 90%, about 91%, about 92%, about 93%, about 94%, about 95%, about 96%, about 97%, about 98%, or about 99% identical to the polynucleotide sequence encoding a polypeptide disclosed herein.

Polynucleotides disclosed herein can be at least about 5, at least about 10, at least about 15, at least about 20, at least about 25, at least about 30, at least about 40, at least about 50, at least about 100, at least about 200, at least about 300, at least about 400, at least about 500, at least about 1000, at least about 5000, at least about 10000, or at least about 15000 or more nucleotides in length, including all values and subvalues therebetween.

Polynucleotide molecules comprising a desired polynucleotide sequence are propagated by placing the nucleic acid molecule encoding the polypeptide molecule in a vector. Viral and non-viral vectors are used, including plasmids. The choice of plasmid will depend on the type of cell in which propagation is desired and the purpose of propagation. Certain vectors are useful for amplifying and making large amounts of the desired DNA sequence. Other vectors are suitable for expression in cells in culture. Still other vectors are suitable for transfer and expression in cells in a whole animal or person. The choice of appropriate vector is well within the skill of the art. Many such vectors are available commercially. The partial or full-length polynucleotide is inserted into a vector typically by means of DNA ligase attachment to a cleaved restriction enzyme site in the vector. Alternatively, the desired nucleotide sequence can be inserted by homologous recombination in vivo. Typically, this is accomplished by attaching regions of homology to the vector on the flanks of the desired nucleotide sequence. Regions of homology are added by ligation of oligonucleotides, or by polymerase chain reaction using primers comprising both the region of homology and a portion of the desired nucleotide sequence, for example.

For expression, an expression cassette or system may be employed. To express a nucleic acid encoding a polypeptide disclosed herein, a nucleic acid molecule encoding the polypeptide, operably linked to regulatory sequences that control transcriptional expression in an expression vector, is introduced into a host cell. In addition to transcriptional regulatory sequences, such as promoters and enhancers, expression vectors can include translational regulatory sequences and a marker gene which is suitable for selection of cells that carry the expression vector. The gene product encoded by a polynucleotide of the disclosure is expressed in any convenient expression system, including, for example, bacterial, yeast, insect, amphibian and mammalian systems. In the expression vector, the polypeptide-encoding polynucleotide is linked to a regulatory sequence as appropriate to obtain the desired expression properties. These can include promoters, enhancers, terminators, operators, repressors, untranslated regions (UTRs), signal sequences, Kozak sequences, polyadenylation signals, internal ribosome entry sites (IRES), and inducers. The promoters can be regulated (e.g., the promoter from the steroid inducible pIND vector (Invitrogen)) or constitutive (e.g., promoters from CMV, SV40, Elongation Factor, or LTR sequences). These are linked to the desired nucleotide sequence using the techniques described above for linkage to vectors. Any techniques known in the art can be used. Accordingly, the expression vector will generally provide a transcriptional and translational initiation region, which can be inducible or constitutive, where the coding region is operably linked under the transcriptional control of the transcriptional initiation region, and a transcriptional and translational termination region.

In some embodiments, polynucleotides encoding the polypeptide molecules described herein are operably linked to a promoter and/or an enhancer. The term “promoter” as used herein refers to a recognition site of a polynucleotide (DNA or RNA) to which an RNA polymerase binds. An RNA polymerase initiates and transcribes polynucleotides operably linked to the promoter. In some embodiments, promoters operative in mammalian cells comprise an AT-rich region located approximately 25 to 30 bases upstream from the site where transcription is initiated and/or another sequence found 70 to 80 bases upstream from the start of transcription, a CNCAAT region where N may be any nucleotide. The term “enhancer” refers to a segment of DNA which contains sequences capable of providing enhanced transcription and in some instances can function independent of their orientation relative to another control sequence. An enhancer can function cooperatively or additively with promoters and/or other enhancer elements.

Non-limiting examples of suitable eukaryotic promoters (promoters functional in a eukaryotic cell) include those from cytomegalovirus (CMV) immediate early, herpes simplex virus (HSV) thymidine kinase, a viral simian virus 40 (SV40) (e.g., early and late SV40), a spleen focus forming virus (SFFV) promoter, long terminal repeats (LTRs) from retrovirus (e.g., a Moloney murine leukemia virus (MoMLV) LTR promoter or a Rous sarcoma virus (RSV) LTR), a herpes simplex virus (HSV) (thymidine kinase) promoter, H5, P7.5, and P11 promoters from vaccinia virus, an elongation factor 1-alpha (EF1α) promoter, early growth response 1 (EGR1) promoter, a ferritin H (FerH) promoter, a ferritin L (FerL) promoter, a Glyceraldehyde 3-phosphate dehydrogenase (GAPDH) promoter, a eukaryotic translation initiation factor 4A1 (EIF4A1) promoter, a heat shock 70 kDa protein 5 (HSPA5) promoter, a heat shock protein 90 kDa beta, member 1 (HSP90B1) promoter, a heat shock protein 70 kDa (HSP70) promoter, a β-kinesin (β-KIN) promoter, the human ROSA 26 locus (Irions et al., Nature Biotechnology 25, 1477-1482 (2007)), a Ubiquitin C (UBC) promoter, a phosphoglycerate kinase-1 (PGK) promoter, a cytomegalovirus enhancer/chicken β-actin (CAG) promoter, a β-actin promoter and a myeloproliferative sarcoma virus enhancer, negative control region deleted, dl587rev primer binding site substituted (MND) promoter, and mouse metallothionein-1. Selection of the appropriate vector and promoter is well within the level of ordinary skill in the art.

In some embodiments, the polynucleotide encoding the polypeptide molecule described herein is operably linked to a constitutive or ubiquitous promoter.

In some embodiments, the polynucleotide encoding the polypeptide molecule described herein is operably linked to an inducible promoter. In such embodiments, the polynucleotide encoding the polypeptide molecule described herein is conditionally expressed. As used herein, “conditional expression” refers to any type of conditional expression including, but not limited to, inducible expression, repressible expression, or expression in specific cells or tissues having a particular physiological, biological, or disease state. Illustrative examples of inducible promoters/systems include, but are not limited to, steroid-inducible promoters such as promoters for genes encoding glucocorticoid or estrogen receptors (inducible by treatment with the corresponding hormone), metallothionine promoter (inducible by treatment with various heavy metals), MX-1 promoter (inducible by interferon), the “GeneSwitch” mifepristone-regulatable system (Sirin et al., 2003, Gene, 323:67), the cumate inducible gene switch (WO 2002/088346), tetracycline-dependent regulatory systems, etc.

In some embodiments, the polynucleotides encoding the polypeptide molecules described herein further comprise a transcription termination signal. Elements directing the efficient termination and polyadenylation of the heterologous nucleic acid transcripts increases heterologous gene expression. Transcription termination signals are generally found downstream of the polyadenylation signal. The term “polyA site” or “polyA sequence” as used herein denotes a DNA sequence which directs both the termination and polyadenylation of the nascent RNA transcript by RNA polymerase II. Polyadenylation sequences can promote mRNA stability by addition of a polyA tail to the 3′ end of the coding sequence and thus, contribute to increased translational efficiency. Cleavage and polyadenylation is directed by a poly(A) sequence in the RNA. The core poly(A) sequence for mammalian pre-mRNAs has two recognition elements flanking a cleavage-polyadenylation site. Typically, an almost invariant AAUAAA hexamer lies 20-50 nucleotides upstream of a more variable element rich in U or GU residues. Cleavage of the nascent transcript occurs between these two elements and is coupled to the addition of up to 250 adenosines to the 5′ cleavage product. In some embodiments, the core poly(A) sequence is an ideal polyA sequence (e.g., AATAAA, ATTAAA, AGTAAA). In some embodiments, the poly(A) sequence is an SV40 polyA sequence, a bovine growth hormone polyA sequence (BGHpA), a rabbit β-globin polyA sequence (rpgpA), variants thereof, or another suitable heterologous or endogenous polyA sequence known in the art.

In some embodiments, a vector may also comprise a sequence encoding a signal peptide (e.g., for nuclear localization, nucleolar localization, mitochondrial localization), fused to the polynucleotide encoding the polypeptide molecules. For example, a vector may comprise a nuclear localization sequence (e.g., from SV40) fused to the polynucleotide encoding the polypeptide molecules. In some embodiments, the signal peptide is an Igk signal peptide. In some embodiments, the signal peptide is a CD8 signal peptide.

In some embodiments, the polynucleotide encoding the polypeptide molecule described herein comprises a nucleotide sequence encoding a protein tag (e.g., 6×His tag, hemagglutinin tag, green fluorescent protein, etc.) that is fused to the site-directed modifying polypeptide, thus resulting in a chimeric polypeptide.

An expression cassette encoding any of the polypeptide molecules of the present disclosure can be introduced into a variety of vectors, e.g., plasmid, BAC, YAC, bacteriophage such as lambda, P1, M13, etc., plant or animal viral vectors (e.g., retroviral-based vectors, adenovirus vectors), and the like, where the vectors are normally characterized by the ability to provide selection of cells comprising the expression vectors. The vectors can provide for extrachromosomal maintenance, particularly as plasmids or viruses, or for integration into the host chromosome. Where extrachromosomal maintenance is desired, an origin sequence is provided for the replication of the plasmid, which can be low- or high copy-number. A wide variety of markers are available for selection, particularly those which protect against toxins, more particularly against antibiotics. The particular marker that is chosen is selected in accordance with the nature of the host, where, in some cases, complementation can be employed with auxotrophic hosts. Introduction of the DNA construct can use any convenient method, including, e.g., conjugation, bacterial transformation, calcium-precipitated DNA, electroporation, fusion, transfection, infection with viral vectors, biolistics, and the like.

Vectors of the present disclosure include any suitable vectors derived from either a eukaryotic or prokaryotic sources. In some embodiments, vectors are obtained from bacteria (e.g., E. coli), insects, yeast (e.g., Pichia pastoris), algae, or mammalian sources.

Exemplary bacterial vectors include, but are not limited to, pACYC177, pASK75, pBAD vector series, pBADM vector series, pET vector series, pETM vector series, pGEX vector series, pHAT, pHAT2, pMal-c2, pMal-p2, pQE vector series, pRSET A, pRSET B, pRSET C, pTrcHis2 series, pZA31-Luc, pZE21-MCS-1, pFLAG ATS, pFLAG CTS, pFLAG MAC, pFLAG Shift-12c, pTAC-MAT-1, pFLAG CTC, or pTAC-MAT-2.

Exemplary insect vectors include, but are not limited to, pFastBacl, pFastBac DUAF, pFastBac ET, pFastBac HTa, pFastBac HTb, pFastBac HTc, pFastBac M30a, pFastBact M3 Ob, pFastBac, M30c, pVF1392, pVF1393, pVF1393 M10, pVF1393 Mi 1, pVF1393 M12, FFAG vectors such as pPolh-FFAGl or pPolh-MAT 2, or MAT vectors such as pPolh-MAT 1, or pPolh-MAT2.

Exemplary yeast vectors include, but are not limited to, Gateway® pDEST™ 14 vector, Gateway® pDEST™ 15 vector, Gateway® pDEST™ 17 vector, Gateway® pDEST™ 24 vector, Gateway® pYES-DEST52 vector, pBAD-DEST49 Gateway® destination vector, pA0815 Pichia vector, pFFDl Pichi pastoris vector, pGAPZA,B, & C Pichia pastoris vector, pPIC3.5K Pichia vector, pPIC6 A, B, & C Pichia vector, pPIC9K Pichia vector, pTEFl/Zeo, pYES2 yeast vector, pYES2/CT yeast vector, pYES2/NT A, B, and C yeast vector, or pYES3/CT yeast vector.

Exemplary algae vectors include pChlamy-4 vector or MCS vector.

Mammalian vectors include transient expression vectors or stable expression vectors. Exemplary mammalian transient expression vectors include, but are not limited to, pRK5, p3xFFAG-CMV 8, pFFAG-Myc-CMV 19, pFEAG-Myc-CMV 23, pFEAG-CMV 2, pFEAG-CMV 6(a-c), pFEAG-CMV 5.1, pFEAG-CMV 5(a-c), p3xFFAG-CMV 7.1, pFEAG-CMV 20, p3xFFAG-Myc-CMV 24, pCMV-FEAG-MAT 1, pCMV-FFAG-MAT2, pBICEP-CMV 3, or pBICEP-CMV 4. Mammalian stable expression vectors include, but are not limited to, pFFAG-CMV 3, p3xFFAG-CMV 9, p3xFFAG-CMV 13, pFFAG-Myc-CMV 21, p3xFFAG-Myc-CMV 25, pFEAG-CMV 4, p3xFFAG-CMV 10, p3xFFAG-CMV 14, pFFAG-Myc-CMV 22, p3xFFAG-Myc-CMV 26, pBICEP-CMV 1, or pBICEP-CMV 2.

In some embodiments, a cell-free system is a mixture of cytoplasmic and/or nuclear components from a cell and is used for in vitro nucleic acid synthesis. In some cases, a cell-free system utilizes either prokaryotic cell components or eukaryotic cell components. For example, a nucleic acid synthesis is obtained in a cell-free system based on Drosophila cells, Xenopus egg, or HeFa cells. Exemplary cell-free systems include, but are not limited to, E. coli S30 Extract system, E. coli T7 S30 system, or PURExpress®.

Accordingly, polypeptide molecules for use within the present disclosure can be produced in genetically engineered host cells according to conventional techniques. Suitable host cells are those cell types that can be transformed or transfected with exogenous DNA and grown in culture, and include bacteria, fungal cells, and cultured higher eukaryotic cells (including cultured cells of multicellular organisms), particularly cultured mammalian cells. Techniques for manipulating cloned DNA molecules and introducing exogenous DNA into a variety of host cells are disclosed by Sambrook and Russell, Molecular Cloning: A Laboratory Manual (3rd ed., Cold Spring Harbor Laboratory Press, Cold Spring Harbor, NY, 2001), and Ausubel et al., Short Protocols in Molecular Biology (4th ed., John Wiley & Sons, 1999).

For example, for recombinant expression of a heterodimeric binding protein comprising different first and second polypeptide chains, the first and second polypeptide chains can be co-expressed from separate vectors in the host cell for expression of the entire heterodimeric protein. Alternatively, for the expression of heterodimeric binding proteins the first and second polypeptide chains are co-expressed from separate expression units in the same vector in the host cell for expression of the entire heterodimeric protein. The expression vector(s) are transferred to a host cell by conventional techniques, and the transfected cells are then cultured by conventional techniques to produce the encoded polypeptide(s) to produce the corresponding polypeptide molecules (e.g., TCRβ-binding domain polypeptides, TAA-binding domain polypeptides, and/or polypeptide complexes thereof).

To direct a recombinant protein into the secretory pathway of a host cell, a secretory signal sequence (also known as a leader sequence) is provided in the expression vector. The secretory signal sequence can be that of the native form of the recombinant protein, or can be derived from another secreted protein or synthesized de novo. The secretory signal sequence is operably linked to the polypeptide-encoding DNA sequence, i.e., the two sequences are joined in the correct reading frame and positioned to direct the newly synthesized polypeptide into the secretory pathway of the host cell. Secretory signal sequences are commonly positioned 5′ to the DNA sequence encoding the polypeptide of interest, although certain signal sequences can be positioned elsewhere in the DNA sequence of interest (see, e.g., U.S. Pat. Nos. 5,037,743 and 5,143,830).

Cultured mammalian cells are suitable hosts for production of polypeptide molecules of the present disclosure. Methods for introducing exogenous DNA into mammalian host cells include calcium phosphate-mediated transfection (Wigler et al., Cell 14:725, 1978; Corsaro and Pearson, Somatic Cell Genetics 7:603, 1981: Graham and Van der Eb, Virology 52:456, 1973), electroporation (Neumann et al., EMBO J. 1:841-845, 1982), DEAE-dextran mediated transfection (Ausubel et al., supra), and liposome-mediated transfection (Hawley-Nelson et al., Focus 15:73, 1993; Ciccarone et al., Focus 15:80, 1993). The production of recombinant polypeptides in cultured mammalian cells is disclosed by, for example, U.S. Pat. Nos. 4,713,339; 4,784,950; 4,579,821; and 4,656,134. Examples of suitable mammalian host cells include, but are not limited to, 293A cell line, 293FT cell line, 293F cells, 293 H cells, CHO DG44 cells, CHO-S cells, CHO-K1 cells, FUT8 KO CHOK1, Expi293F™ cells, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REx™ Jurkat cell line, Per.C6 cells, T-REx™-293 cell line, T-REx™-CHO cell line, and T-REx™-HeLa cell line. Additional suitable cell lines are known in the art and available from public depositories such as the American Type Culture Collection, Manassas, Virginia. Strong transcription promoters can be used, such as promoters from SV-40 or cytomegalovirus. See, e.g., U.S. Pat. No. 4,956,288. Other suitable promoters include those from metallothionein genes (U.S. Pat. Nos. 4,579,821 and 4,601,978) and the adenovirus major late promoter.

Drug selection is generally used to select for cultured mammalian cells into which foreign DNA has been inserted. Such cells are commonly referred to as “transfectants.” Cells that have been cultured in the presence of the selective agent and are able to pass the gene of interest to their progeny are referred to as “stable transfectants.” Exemplary selectable markers include a gene encoding resistance to the antibiotic neomycin, which allows selection to be carried out in the presence of a neomycin-type drug, such as G-418 or the like; the gpt gene for xanthine-guanine phosphoribosyl transferase, which permits host cell growth in the presence of mycophenolic acid/xanthine; and markers that provide resistance to zeocin, bleomycin, blastocidin, and hygromycin (see, e.g., Gatignol et al., Mol. Gen. Genet. 207:342, 1987; Drocourt et al., Nucl. Acids Res. 18:4009, 1990). Selection systems can also be used to increase the expression level of the gene of interest, a process referred to as “amplification.” Amplification is carried out by culturing transfectants in the presence of a low level of the selective agent and then increasing the amount of selective agent to select for cells that produce high levels of the products of the introduced genes. An exemplary amplifiable selectable marker is dihydrofolate reductase, which confers resistance to methotrexate. Other drug resistance genes (e.g., hygromycin resistance, multi-drug resistance, puromycin acetyltransferase) can also be used.

Other higher eukaryotic cells can also be used as hosts, including insect cells, plant cells and avian cells. The use of Agrobacterium rhizogenes as a vector for expressing genes in plant cells has been reviewed by Sinkar et al., J. Biosci. (Bangalore) 11:47-58, 1987. Transformation of insect cells and production of foreign polypeptides therein is disclosed in U.S. Pat. No. 5,162,222 and PCT Publication No. WO 94/06463. Exemplary insect cells include, but are not limited to, Drosophila S2 cells, Sf9 cells, Sf21 cells, High Five™ cells, and expresSF+® cells; and exemplary algae cells include, but are not limited to Chlamydomonas reinhardtii 137c.

Insect cells can be infected with recombinant baculovirus, commonly derived from Autographa californica nuclear polyhedrosis virus (AcNPV). See King and Possee, The Baculovirus Expression System: A Laboratory Guide (Chapman & Hall, London); O'Reilly et al., Baculovirus Expression Vectors: A Laboratory Manual (Oxford University Press., New York 1994); and Baculovirus Expression Protocols. Methods in Molecular Biology (Richardson ed., Humana Press, Totowa, NJ, 1995). Recombinant baculovirus can also be produced through the use of a transposon-based system described by Luckow et al. (J. Virol. 67:4566-4579, 1993). This system, which utilizes transfer vectors, is commercially available in kit form (BAC-TO-BAC kit; Life Technologies, Gaithersburg, MD). The transfer vector (e.g., PFASTBAC1; Life Technologies) contains a Tn7 transposon to move the DNA encoding the protein of interest into a baculovirus genome maintained in E. coli as a large plasmid called a “bacmid.” See Hill-Perkins and Possee, J. Gen. Virol. 71:971-976, 1990; Bonning et al., J. Gen. Virol. 75:1551-1556, 1994; and Chazenbalk and Rapoport, J. Biol. Chem. 270:1543-1549, 1995. In addition, transfer vectors can include an in-frame fusion with DNA encoding a polypeptide extension or affinity tag as disclosed above. Using techniques known in the art, a transfer vector containing a protein-encoding DNA sequence is transformed into E. coli host cells, and the cells are screened for bacmids which contain an interrupted lacZ gene indicative of recombinant baculovirus. The bacmid DNA containing the recombinant baculovirus genome is isolated, using common techniques, and used to transfect Spodoptera frugiperda cells, such as Sf9 cells. Recombinant virus that expresses the protein or interest is subsequently produced. Recombinant viral stocks are made by methods commonly used in the art.

For protein production, the recombinant virus is used to infect host cells, typically a cell line derived from the fall armyworm, Spodoptera frugiperda (e.g., Sf9 or Sf21 cells) or Trichoplusia ni (e.g., HIGH FIVE™ cells; Invitrogen, Carlsbad, CA). See generally Glick and Pasternak, Molecular Biotechnology, Principles & Applications of Recombinant DNA (ASM Press, Washington, D.C., 1994). See also U.S. Pat. No. 5,300,435. Serum-free media are used to grow and maintain the cells. Suitable media formulations are known in the art and can be obtained from commercial suppliers.

Fungal cells, including yeast cells, can also be used to produce the polypeptide molecules of the present disclosure. Exemplary yeast or filamentous fungi include, for example, the genus: Saccharomyces, Schizosaccharomyces, Candida, Pichia, Hansenula, Kluyveromyces, Zygosaccharomyces, Yarrowia, Trichosporon, Rhodosporidi, Aspergillus, Fusarium, or Trichoderma. Yeast species of in this regard include, e.g., Saccharomyces cerevisiae, Schizosaccharomyces pombe, Candida utilis, Candida boidini, Candida albicans, Candida tropicalis, Candida stellatoidea, Candida glabrata, Candida krusei, Candida parapsilosis, Candida guilliermondii, Candida viswanathii, Candida lusitaniae, Rhodotorula mucilaginosa, Pichia metanolica, Pichia angusta, Pichia pastoris, Pichia anomala, Hansenula polymorpha, Kluyveromyces lactis, Zygosaccharomyces rouxii, Yarrowia lipolytica, Trichosporon pullulans, Rhodosporidium toru-Aspergillus niger, Aspergillus nidulans, Aspergillus awamori, Aspergillus oryzae, Trichoderma reesei, Yarrowia lipolytica, Brettanomyces bruxellensis, Candida stellata, Schizosaccharomyces pombe, Torulaspora delbrueckii, Zygosaccharomyces bailii, Cryptococcus neoformans, Cryptococcus gattii, or Saccharomyces boulardii. Exemplary Pichia pastoris strains include GS 115, KM71H, SMD1168, SMD1168H, and X-33; and the Saccharomyces cerevisiae strain INVScl.

Methods for transforming yeast cells (e.g., S. cerevisiae) with exogenous DNA and producing recombinant polypeptides therefrom are disclosed by, for example, U.S. Pat. Nos. 4,599,311; 4,931,373; 4,870,008; 5,037,743; and 4,845,075. Transformed cells are selected by phenotype determined by the selectable marker, commonly drug resistance or the ability to grow in the absence of a particular nutrient (e.g., leucine). An exemplary vector system for use in Saccharomyces cerevisiae is the POT1 vector system disclosed by Kawasaki et al. (U.S. Pat. No. 4,931,373), which allows transformed cells to be selected by growth in glucose-containing media. Suitable promoters and terminators for use in yeast include those from glycolytic enzyme genes (see, e.g., U.S. Pat. Nos. 4,599,311; 4,615,974; and 4,977,092) and alcohol dehydrogenase genes. See also U.S. Pat. Nos. 4,990,446; 5,063,154; 5,139,936; and 4,661,454. Transformation systems for other yeasts, including Hansenula polymorpha, Schizosaccharomyces pombe, Kluyveromyces lactis, Kluyveromyces fragilis, Ustilago maydis, Pichia pastoris, Pichia methanolica, Pichia guillermondii, and Candida maltosa are known in the art. See, e.g., Gleeson et al., J. Gen. Microbiol. 132:3459-3465, 1986; U.S. Pat. No. 4,882,279; and Raymond et al., Yeast 14:11-23, 1998. Aspergillus cells can be utilized according to the methods of McKnight et al., U.S. Pat. No. 4,935,349. Methods for transforming Acremonium chrysogenum are disclosed by Sumino et al., U.S. Pat. No. 5,162,228. Methods for transforming Neurospora are disclosed by Lambowitz, U.S. Pat. No. 4,486,533. Production of recombinant proteins in Pichia methanolica is disclosed in U.S. Pat. Nos. 5,716,808; 5,736,383; 5,854,039; and 5,888,768.

Prokaryotic host cells, including strains of the bacteria Escherichia coli, Bacillus, and other genera are also useful host to produce the polypeptide molecules of the present disclosure. Techniques for transforming these hosts and expressing foreign DNA sequences cloned therein are well-known in the art (see, e.g., Sambrook and Russell, supra). When expressing a recombinant protein in bacteria such as E. coli, the protein can be retained in the cytoplasm, typically as insoluble granules, or can be directed to the periplasmic space by a bacterial secretion sequence. In the former case, the cells are lysed, and the granules are recovered and denatured using, for example, guanidine isothiocyanate or urea. The denatured protein can then be refolded and dimerized by diluting the denaturant, such as by dialysis against a solution of urea and a combination of reduced and oxidized glutathione, followed by dialysis against a buffered saline solution. In the alternative, the protein can be recovered from the cytoplasm in soluble form and isolated without the use of denaturants. The protein is recovered from the cell as an aqueous extract in, for example, phosphate buffered saline. To capture the protein of interest, the extract is applied directly to a chromatographic medium, such as an immobilized antibody or heparin-Sepharose column. Secreted proteins can be recovered from the periplasmic space in a soluble and functional form by disrupting the cells (by, for example, sonication or osmotic shock) to release the contents of the periplasmic space and recovering the protein, thereby obviating the need for denaturation and refolding. Antibodies, including single-chain antibodies, can be produced in bacterial host cells according to known methods. See, e.g., Bird et al., Science 242:423-426, 1988; Huston et al., Proc. Natl. Acad. Sci. USA 85:5879-5883, 1988; and Pantoliano et al., Biochem. 30:10117-10125, 1991.

Transformed or transfected host cells to produce the polypeptide molecules of the present disclosure are cultured according to conventional procedures in a culture medium containing nutrients and other components required for the growth of the chosen host cells. A variety of suitable media, including defined media and complex media, are known in the art and generally include a carbon source, a nitrogen source, essential amino acids, vitamins and minerals. Media can also contain such components as growth factors or serum, as required. The growth medium will generally select for cells containing the exogenously added DNA by, for example, drug selection or deficiency in an essential nutrient which is complemented by the selectable marker carried on the expression vector or co-transfected into the host cell.

The polypeptide molecules of the present disclosure may be purified by conventional protein purification methods, typically by a combination of chromatographic techniques. See generally Affinity Chromatography: Principles & Methods (Pharmacia LKB Biotechnology, Uppsala, Sweden, 1988); Scopes, Protein Purification: Principles and Practice (Springer-Verlag, New York 1994). Proteins comprising an immunoglobulin Fc region can be purified by affinity chromatography on immobilized protein A or protein G. In some embodiments, the polypeptide molecules of the present disclosure are purified, for example, by chromatography (e.g., ion exchange, affinity, particularly by affinity for the specific antigen after Protein A, and sizing column chromatography), centrifugation, differential solubility. Additional purification steps, such as gel filtration, can be used to obtain the desired level of purity or to provide for desalting, buffer exchange, and the like.

### Pharmaceutical Compositions

In some embodiments, the present disclosure provides pharmaceutical compositions comprising polypeptide molecules disclosed herein (e.g., polypeptide molecules comprising a T cell-binding domain and/or a TAA-binding domain), and a pharmaceutically acceptable carrier, diluent, or excipient.

In some embodiments, the pharmaceutical composition comprises a polypeptide molecule, wherein the polypeptide molecule comprises a TCRβ-binding domain. In some embodiments, the pharmaceutical composition comprises a polypeptide molecule, wherein the polypeptide molecule comprises a TCRβV-binding domain. In some embodiments, the pharmaceutical composition comprises a polypeptide molecule, wherein the polypeptide molecule comprises a TCRβV6-5-binding domain. In some embodiments, the pharmaceutical composition comprises a polypeptide molecule, wherein the polypeptide molecule comprises a TCRβV12-3-binding domain. In some embodiments, the pharmaceutical composition comprises a polypeptide molecule, wherein the polypeptide molecule comprises a TAA-binding domain (e.g., a CD19-binding domain). In some embodiments, the pharmaceutical composition comprises at least one pharmaceutically acceptable carrier, diluent, or excipient.

In some embodiments, the present disclosure provides a pharmaceutical composition comprising a polypeptide molecule, wherein the polypeptide molecule comprises at least one-binding domain. In some embodiments, the present disclosure provides a pharmaceutical composition comprising a polypeptide molecule, wherein the polypeptide molecule comprises a first binding domain and a second binding domain. In some embodiments, the first binding domain is a TCRβV-binding domain (e.g., a TCRβV6-5-binding domain or a TCRβV12-3-binding domain). In some embodiments, the second binding domain is a tumor-associated antigen binding domain (e.g., CD19-binding domain). In some embodiments, the pharmaceutical composition comprises at least one pharmaceutically acceptable carrier, diluent, or excipient.

In some embodiments, the pharmaceutical composition comprises a polynucleotide encoding the polypeptide molecules described herein, and a pharmaceutically acceptable carrier, diluent, or excipient. In some embodiments, the pharmaceutical composition comprises a vector encoding the polypeptide molecules described herein, and a pharmaceutically acceptable carrier, diluent, or excipient.

Pharmaceutically acceptable carriers, diluents, or excipients include, without limitation, any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, and/or emulsifier which has been approved by the United States Food and Drug Administration as being acceptable for use in humans and/or domestic animals.

The pharmaceutical compositions described herein are formulated according to known methods to prepare pharmaceutically useful compositions, whereby the polypeptide molecules are combined in a mixture with a pharmaceutically acceptable carrier, diluent or excipient. Suitable carriers, diluents, excipients, etc. can be found in standard pharmaceutical texts. See, for example, Handbook of Pharmaceutical Additives, 2nd Edition (eds. M. Ash and I. Ash), 2001 (Synapse Information Resources, Inc., Endicott, New York, USA), Remington's Pharmaceutical Sciences, 20th edition, pub. Lippincott, Williams & Wilkins, 2000; and Handbook of Pharmaceutical Excipients, 2nd edition, 1994. The precise nature of the carrier, diluent, or excipient will depend on the route of administration, which may be oral, or by injection, e.g., cutaneous, subcutaneous, or intravenous.

In some embodiments, the pharmaceutical compositions described herein are formulated in one of the following dosage forms: an intravenous dosage form, an intramuscular dosage form, an intraperitoneal dosage form, a subcutaneous dosage form, an oral dosage form, an intranasal dosage form, a suppository dosage form, an intradermal dosage form, or a topical dosage form. In some embodiments, the pharmaceutical compositions described herein are formulated in an intravenous dosage form. In some embodiments, the pharmaceutical compositions described herein are formulated in a subcutaneous dosage form. In some embodiments, the pharmaceutical compositions described herein are formulated in an oral dosage form.

Exemplary pharmaceutically acceptable carriers include, but are not limited to, sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter; waxes; animal and vegetable fats; paraffins; silicones; bentonites; silicic acid; zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations. Except insofar as any conventional media and/or agent is incompatible with the agents of the present disclosure, its use in therapeutic compositions is contemplated. Supplementary active ingredients also can be incorporated into the compositions.

Pharmaceutically acceptable salts include both acid and base addition salts. Pharmaceutically acceptable salts include the acid addition salts (formed with the free amino groups of the protein) and which are formed with inorganic acids such as, for example, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, ptoluenesulfonic acid, trifluoroacetic acid, undecylenic acid, and the like. Salts formed with the free carboxyl groups can also be derived from inorganic bases such as, for example, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. In some embodiments, organic bases are isopropylamine, diethylamine, ethanolamine, trimethylamine, dicyclohexylamine, choline and caffeine.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The pharmaceutical compositions disclosed herein can be provided as a controlled release or sustained release system. In some embodiments, a pump may be used to achieve controlled or sustained release (see, e.g., Sefton, Crit. Ref. Biomed. Eng. 14: 201-40 (1987); Buchwald et al., Surgery 88: 507-16 (1980); and Saudek et al., N. Engl. J. Med. 321: 569-74 (1989)). In some embodiments, polymeric materials can be used to achieve controlled or sustained release of a prophylactic or therapeutic agent or a composition provided herein (see, e.g., Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 23: 61-126 (1983); Levy et al., Science 228: 190-92 (1985); During et al., Ann. Neurol. 25: 351-56 (1989); Howard et al., J. Neurosurg. 71: 105-12 (1989); U.S. Pat. Nos. 5,679,377; 5,916,597; 5,912,015; 5,989,463; and 5,128,326; PCT Publication Nos. WO 99/15154 and WO 99/20253). Examples of polymers used in sustained release formulations include, but are not limited to, poly (2-hydroxy ethyl methacrylate), poly (methyl methacrylate), poly (acrylic acid), poly (ethylene-co-vinyl acetate), poly (methacrylic acid), polyglycolides (PLG), polyanhydrides, poly (N-vinyl pyrrolidone), poly (vinyl alcohol), polyacrylamide, poly (ethylene glycol), polylactides (PLA), poly (lactide-co-glycolides) (PLGA), and polyorthoesters. In some embodiments, the polymer used in a sustained release formulation is inert, free of leachable impurities, stable on storage, sterile, and biodegradable. In some embodiments, a controlled or sustained release system can be placed in proximity of a particular target tissue, for example, the kidney, thus requiring only a fraction of the systemic dose (see, e.g., Goodson, Medical Applications of Controlled Release Vol. 2, 115-38 (1984)). Controlled release systems are discussed, for example, by Langer, Science 249: 1527-33 (1990). Any technique known to one of skill in the art can be used to produce sustained release formulations comprising one or more agents as described herein (see, e.g., U.S. Pat. No. 4,526,938, PCT publication Nos. WO 91/05548 and WO 96/20698, Ning et al., Radiotherapy & Oncology 39: 179-89 (1996); Song et al., PDA J. of Pharma. Sci. & Tech. 50: 372-97 (1995); Cleek et al., Pro. Int'l. Symp. Control. Rel. Bioact. Mater. 24: 853-54 (1997); and Lam et al., Proc. Int'l. Symp. Control Rel. Bioact. Mater. 24: 759-60 (1997)).

The polypeptide molecules may also be entrapped in microcapsules prepared, for example, by coascervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 18th edition.

In some embodiments, the pharmaceutical compositions disclosed herein to are sterile. The pharmaceutical composition may be rendered sterile by filtration through sterile filtration membranes. The pharmaceutical compositions disclosed herein generally can be placed into a container having a sterile access port, for example, an intravenous solution bag or vial having a stopper pierceable by a hypodermic injection needle.

### Methods of Use

The present disclosure provides methods of using any of the polypeptide molecules disclosed herein, such as using the polypeptide molecules for preventing or treating a disease, disorder, or condition in a subject in need thereof. In some embodiments, the present disclosure provides a method of preventing or treating a disease, disorder, or condition in a subject in need thereof, comprising administering a therapeutically effective amount of the polypeptide molecule or a pharmaceutical composition thereof to the subject. In some embodiments, the polypeptide molecule comprises a TCRβV-binding domain. In some embodiments, the polypeptide molecule comprises a TCRβC-binding domain. In some embodiments, the polypeptide molecule comprises a TAA-binding domain. In some embodiments, the polypeptide molecule comprises TCRβ-binding domain and a TAA-binding domain.

Administration of the polypeptide molecule or pharmaceutical composition thereof can occur by infusion (e.g., continuous or bolus), injection, irrigation, inhalation, consumption, electro-osmosis, hemodialysis, iontophoresis, and other methods known in the art.

In some embodiments, the polypeptide molecule or pharmaceutical composition thereof is administered to a subject in need thereof by one or more administration routes. Possible administration routes include, for example, intravenous, intramuscular, subcutaneous, intraperitoneal, oral, intranasal, intrasynovial, parenteral, intrapulmonary, transdermal, intrathecal, topical, intraarterial, intracranial, intradermal, intraduodenal, intrammamary, intrameningeal, intratumoral, intravitreal, ophthalmic, spinal, ureteral, urethral, vaginal, intrauterine, and intralesional routes. In some embodiments, the administration route is selected from the group consisting of: intravenous administration, intramuscular administration, subcutaneous administration, intraperitoneal administration, and oral administration. In some embodiments, the polypeptide molecule is administered intravenously to the subject. In some embodiments, the polypeptide molecule is administered subcutaneously to the subject. In some embodiments, the polypeptide molecule is administered orally to the subject. In some embodiments, administration route is direct, local, or systemic.

For prevention and treatment purposes, the polypeptide molecules or pharmaceutical compositions thereof can be administered to a subject in a single bolus delivery, via continuous delivery (e.g., continuous transdermal delivery) over an extended time period, or in a repeated administration protocol (e.g., on an hourly, daily, weekly, monthly, or yearly basis).

In some embodiments, the methods provided herein comprise administering a therapeutically effective dose of the polypeptide molecule or pharmaceutical composition thereof to a subject in need thereof. As used herein, “therapeutically effective dose” or a “therapeutically effective amount” means a dose, dosage, or amount sufficient to achieve the intended therapeutic purpose, such as, to alleviate a sign or symptom of a disease, disorder, or condition in a subject. A therapeutically effective dose, dosage, or amount, as defined above, refers to the amount of polypeptide molecule required to result in a particular physiological effect, e.g., prevention or amelioration of one or more symptoms of cancer. Determination of therapeutically effective dosages in this context are typically based on animal model studies followed by human clinical trials and is guided by determining effective dosages and administration protocols that significantly reduce tumor growth and/or increase survival in model subjects. Effective doses of the polypeptide molecules or pharmaceutical compositions thereof vary depending upon many different factors, including means of administration, target site, physiological state of a subject, whether a subject is human or an animal, other medications administered, whether treatment is prophylactic or therapeutic, as well as the specific activity of the composition itself and its ability to elicit the desired response in a subject. Typically, dosage regimens are adjusted to provide an optimum therapeutic response, i.e., to optimize safety and efficacy in a subject.

Dosage of the pharmaceutical composition comprising the polypeptide molecule can be varied by the attending clinician to maintain a desired concentration. Higher or lower concentrations can be selected based on the mode of delivery. In some embodiments, the polypeptide molecule or pharmaceutical composition thereof is administered at any time during a subject's life.

In some embodiments, the polypeptide molecule or pharmaceutical composition thereof is used to prevent or treat cancer in a subject. In some embodiments, the polypeptide molecule or pharmaceutical composition thereof is used to prevent or treat an autoimmune disorder in a subject. In some embodiments, the polypeptide molecule is used to treat a disease, disorder, or condition associated with overexpression and/or ectopic expression of CD19.

In some embodiments, the polypeptide molecule or pharmaceutical composition thereof is used to deplete unwanted cell types from tissues either in vitro or in vivo. In some embodiments, the polypeptide molecule or pharmaceutical composition thereof is used to modulate immune responses to treat graft-versus-host disease and/or is used in purging transplantation tissues of unwanted cell types. For example, in some embodiments, the polypeptide molecule is used for the purpose of depleting B cells and/or T cells from a donor cell population as a prophylaxis against graft-versus-host disease, and induction of immune tolerance, in a subject to undergo bone marrow and/or stem cell transplantation. In some embodiments, the transplant recipient is administered the polypeptide molecule described herein (e.g., TCRβ-CD19 polypeptide molecule) for prophylaxis or treatment of host-versus-graft disease via the targeted cell-killing of host B cells and/or T cells (see e.g., Sarantopoulos S et al., Biol Blood Marrow Transplant 21: 16-23 (2015)).

In some embodiments, the polypeptide molecule or pharmaceutical composition thereof is used to prevent or treat cancer in a subject. In some embodiments, the cancer is a CD19-positive cancer. In some embodiments, the cancer is a PSMA-positive cancer. In some embodiments, the cancer is characterized by a high mutational burden and/or a high frequency of indels. In some embodiments, the cancer is a solid tumor. In some embodiments, the cancer is metastatic.

In some embodiments, the cancer is a hematologic cancer. In some embodiments, the hematologic cancer is leukemia. In some embodiments, the leukemia is acute myeloid leukemia (AML), treatment-related AML, secondary AML, B and T cell acute lymphoblastic leukemia (ALL), chronic lymphocytic leukemia (CLL), B chronic lymphocytic leukemia (B-CLL), hairy cell leukemia (HCL) chronic lymphocytic leukemia, or chronic myelogenous leukemia (CML). In some embodiments, the hematologic cancer is lymphoma. In some embodiments, the lymphoma is Hodgkin lymphoma, non-Hodgkin lymphoma, primary mediastinal large B-cell lymphoma, Burkitt's lymphoma (BL), T cell lymphoma, mantle cell lymphoma, follicular lymphoma, or diffuse large B-cell lymphoma. In some embodiments, the hematologic cancer is myelodysplastic syndrome (MDS). In some embodiments, the hematologic cancer is Myeloproliferative Disorder (MPD). In some embodiments, the hematologic cancer is myeloma. In some embodiments, the myeloma is multiple myeloma. In some embodiments, the hematologic cancer is chronic lymphocytic leukemia. In some embodiments, the hematologic cancer is non-Hodgkin lymphoma.

In some embodiments, the polypeptide molecules or pharmaceutical compositions thereof are used to prevent or treat an autoimmune disease or autoimmune disorder in a subject. In some embodiments, the autoimmune disease or autoimmune disorder is selected from rheumatoid arthritis, systemic lupus erythematosus, multiple sclerosis, type 1 diabetes, inflammatory bowel disease (e.g., ulcerative colitis or Crohn's disease), psoriasis, psoriatic arthritis, Hashimoto's thyroiditis, Graves' disease, Celiac disease, ankylosing spondylitis, Sjögren's syndrome, myasthenia gravis, scleroderma, autoimmune hepatitis, Addison's disease, vasculitis, pernicious anemia, immune thrombocytopenia purpura (ITP) or idiopathic thrombocytopenia purpura, Felty's syndrome, autoimmune hemolytic anemia, bullous pemphigoid, autoimmune neutropenia, autoimmune polyglandular syndrome type 1, Goodpasture's disease or Goodpasture syndrome, pemphigus vulgaris, pemphigus foliaceus, polychondritis, epidermolysis bullosa acquisita, amyloidosis, asthma, graft rejection, graft-versus-host disease, host-versus-graft disease, hemolytic uremic syndrome, polyarteritis, anti-phospholipid antibody syndrome, sarcoidosis, or other autoimmune disorders in which B cells or pathogenic antibodies produced by B cells contribute to disease pathology.

In some embodiments, the subject with the disease, disorder, or condition is relapsed or refractory to at least one therapy. In some embodiments, the subject with the disease, disorder, or condition is relapsed or refractory to at least one, at least two, at least three, at least four, at least five, or more therapies.

In some embodiments, the subject has previously been treated with a polypeptide molecule comprising a binding domain that specifically binds to CD3 (e.g., an anti-CD3/anti-CD19 polypeptide molecule). In some embodiments, the subject is relapsing to a treatment comprising the polypeptide molecule comprising the CD3-binding domain. In some embodiments, the subject is refractory to a treatment comprising the polypeptide molecule comprising the CD3-binding domain.

In some embodiments, the polypeptide molecule or pharmaceutical composition thereof reduces tumor burden in a subject with cancer. In some embodiments, polypeptide molecule or pharmaceutical composition thereof reduces tumor growth in a subject with cancer. In some embodiments, the polypeptide molecule or pharmaceutical composition thereof reduces or prevents metastases in a subject with cancer. In some embodiments, the polypeptide molecule or pharmaceutical composition thereof increases survival of a subject with cancer.

In some embodiments, the polypeptide molecule binds to an effector cell and a target cell. In some embodiments, binding of the polypeptide molecule to the effector cell and the target cell increases effector cell activation. In some embodiments, binding of the polypeptide molecule to the effector cell and the target cell increases effector cell proliferation. In some embodiments, binding of the polypeptide molecule to the effector cell and the target cell increases effector cell differentiation. In some embodiments, binding of the polypeptide molecule to the effector cell and the target cell increases effector cell cytotoxicity. In some embodiments, binding of the polypeptide molecule to the effector cell and the target cell increases effector cell-mediated lysis of the target cell. In some embodiments, the target cell is a CD19-positive cell. In some embodiments, the target cell is a PSMA-positive cell. In some embodiments, the effector cell is a TCRβ-positive cell. In some embodiments, the effector cell is a TCRβV-positive cell.

In some embodiments, the polypeptide molecule comprises one or more mutations compared to a wildtype or parental polypeptide molecule. In some embodiments, the polypeptide molecule has an increased binding affinity to an antigen compared to a wildtype polypeptide molecule. In some embodiments, binding affinity of the polypeptide molecule is increased by at least about 10%, 30%, 50%, 70%, 90%, 1.5-fold, 2-fold, 3-fold, 4-fold, 5-fold, 10-fold, 50-fold, 100-fold, 200-fold, 300-fold, 400-fold, 500-fold, 600-fold, 700-fold, 800-fold, 900-fold, or 1000-fold, including any range or subrange therebetween, to an antigen compared to a wildtype polypeptide molecule. In some embodiments, the antigen is TCRβC (e.g., TCRβC1 or TCRβC2). In some embodiments, the antigen is TCRβV (e.g., TCRβV6-5 or TCRβV12-3). In some embodiments, the antigen is CD19. In some embodiments, the antigen is PSMA.

### Polypeptide Molecules of the Present Disclosure Exhibit Improved Technical Effects

In some embodiments, the polypeptide molecules of the present disclosure comprise modifications of wildtype or parental sequences to generate improved functionalities. Thus, in some embodiments, the polypeptide molecule comprises one or more mutations compared to a wildtype or parental polypeptide molecule.

In some embodiments, the polypeptide molecule of the present disclosure comprises a binding domain with increased binding affinity of at least 4%, 6%, 8%, 10%, 12%, 14%, 16%, 18%, 20%, 22%, 24%, 26%, 28%, 30%, 32%, 34%, 36%, 38%, 40%, 42%, 44%, 46%, 48%, 50%, 52%, 54%, 56%, 58%, 60%, 62%, 64%, 66%, 68%, 70%, 72%, 74%, 76%, 78%, 80%, 82%, 84%, 86%, 88%, 90%, 92%, 94%, 96%, 98%, 100%, 102%, 104%, 106%, 108%, 110%, 112%, 114%, 116%, 118%, 120%1, 122%, 124%, 126%1, 128%, 130%, 132%, 134%, 136%, 138%, 140%1, 142%, 144%, 146%, 148%1, 150%, 152%, 154%1, 156%, 158%, 160%, 162%, 164%, 166%, 168%1, 170%, 172%, 174%, 176%, 178%, 180%, 182%, 184%, 186%, 188%, 190%, 192%, 194%, 196%, 198%, 200%, 202%, 204%, 206%, 208%, 210%, 212%, 214%, 216%, 218%, 220%, 222%, 224%, 226%, 228%, 230%, 232%, 234%, 236%, 238%, 240%, 242%, 244%, 246%, 248%, 250%, 252%, 254%, 256%, 258%, 260%, 262%, 264%, 266%, 268%, 270%, 272%, 274%, 276%, 278%, 280%, 282%, 284%, 286%, 288%, 290%, 292%, 294%, 296%, 298%, 300%, including any ranges or subranges therebetween to an antigen as compared to the binding affinity of a wildtype or parental binding domain targeting the same antigen. In some embodiments, the antigen is TCRβC (e.g., TCRβC1 or TCRβC2). In some embodiments, the antigen is TCRβV (e.g., TCRβV6-5 or TCRβV12-3). In some embodiments, the antigen is CD19. In some embodiments, the antigen is PSMA. In some embodiments the antigen is human TCRβV6-5. In some embodiments the antigen is cyno TCRβV6-2, cyno TCRβV6-1, cyno-TRβV6-3, and/or cyno-TCRβV6-6.

In some embodiments, the polypeptide molecule comprises one or more mutations compared to a wildtype or parental polypeptide molecule. In some embodiments, the polypeptide molecule comprises a binding domain with increased binding affinity of at least 2-fold, 4-fold, 6-fold, 8-fold, 10-fold, 12-fold, 14-fold, 16-fold, 18-fold, 20-fold, 22-fold, 24-fold, 26-fold, 28-fold, 30-fold, 32-fold, 34-fold, 36-fold, 38-fold, or 40-fold, including any ranges or subranges therebetween, to an antigen as compared to the binding affinity of a wildtype or parental binding domain. In some embodiments, the antigen is TCRβC (e.g., TCRβC1 or TCRβC2). In some embodiments, the antigen is TCRβV (e.g., TCRβV6-5 or TCRβV12-3). In some embodiments, the antigen is CD19. In some embodiments, the antigen is PSMA. In some embodiments the antigen is human TCRβV6-5. In some embodiments the antigen is cyno TCRβV6-2, cyno TCRβV6-1, cyno-TRβV6-3, and/or cyno-TCRβV6-6.

In some embodiments, the bispecific polypeptide molecule of the disclosure with at least two binding domains, has an increased binding affinity to an antigen compared to an otherwise comparable polypeptide molecule lacking bispecificity (i.e., lacking a second binding domain). In some embodiments, the bispecific polypeptide molecule has an increased binding affinity of at least 10%, 30%, 50%, 70%, 90%, 1 time, 2 times, 3 times, 4 times, 5 times, or 10 times, including any ranges or subranges therebetween, to an antigen as compared to the binding affinity of an otherwise comparable polypeptide molecule that lacks bispecificity. In some embodiments, the bispecific polypeptide molecule has an increased binding affinity of at least 4%, 6%, 8%, 10%, 12%, 14%, 16%, 18%, 20%, 22%, 24%, 26%, 28%, 30%, 32%, 34%, 36%, 38%, 40%, 42%, 44%, 46%, 48%, 50%, 52%, 54%, 56%, 58%, 60%, 62%, 64%, 66%, 68%, 70%, 72%, 74%, 76%, 78%, 80%, 82%, 84%, 86%, 88%, 90%, 92%, 94%, 96%, 98%, 100%, 102%, 104%, 106%, 108%, 110%, 112%, 114%, 116%, 118%, 120%, 122%, 124%, 126%, 128%, 130%, 132%, 134%, 136%, 138%, 140%, 142%, 144%, 146%, 148%, 150%, 152%, 154%, 156%, 158%, 160%, 162%, 164%, 166%, 168%, 170%, 172%, 174%, 176%, 178%, 180%, 182%, 184%, 186%, 188%, 190%, 192%, 194%, 196%, 198%, 200%, 202%, 204%, 206%, 208%, 210%, 212%, 214%, 216%, 218%, 220%, 222%, 224%, 226%, 228%, 230%, 232%, 234%, 236%, 238%, 240%, 242%, 244%, 246%, 248%, 250%, 252%, 254%, 256%, 258%, 260%, 262%, 264%, 266%, 268%, 270%, 272%, 274%, 276%, 278%, 280%, 282%, 284%, 286%, 288%, 290%, 292%, 294%, 296%, 298%, or 300%, including any ranges or subranges therebetween, to an antigen as compared to the binding affinity of an otherwise comparable polypeptide molecule that lacks bispecificity. In some embodiments, the bispecific polypeptide molecule has an increased binding affinity of at least 2-fold, 4-fold, 6-fold, 8-fold, 10-fold, 12-fold, 14-fold, 16-fold, 18-fold, 20-fold, 22-fold, 24-fold, 26-fold, 28-fold, 30-fold, 32-fold, 34-fold, 36-fold, 38-fold, or 40-fold, including any range or subrange therebetween, to an antigen as compared to the binding affinity of an otherwise comparable polypeptide molecule that lacks bispecificity. In some embodiments, the antigen is TCRβC (e.g., TCRβC1 or TCRβC2). In some embodiments, the antigen is TCRβV (e.g., TCRβV6-5 or TCRβV12-3). In some embodiments, the antigen is CD19. In some embodiments, the antigen is PSMA. In some embodiments, the antigen is CD19. In some embodiments, the antigen is PSMA. In some embodiments the antigen is human TCRβV6-5. In some embodiments the antigen is cyno TCRβV6-2, cyno TCRβV6-1, cyno-TRβV6-3, and/or cyno-TCRβV6-6.

Provided are also various methods employing modified cells. For example, the polypeptide molecules of the disclosure can be effective at improving a function of a cell. In some embodiments, a cell transformed with a polypeptide molecule can comprise increased binding affinity.

Provided herein are methods of expanding T cells using the polypeptide molecules of the present disclosure. In some embodiments, the method of expanding T cells comprises contacting a T cell with the polypeptide molecule, thereby inducing proliferation or differentiation of a population of T cells in a sample or subject. In some embodiments, the polypeptide molecule comprises at least one binding domain that specifically binds to a T cell receptor beta variable region (TCRβV) expressed on the T cell. In some embodiments, the population of T cells comprises a plurality of memory T cells and effector T cells. In some embodiments, methods comprise expanding T cells in vivo. In some embodiments, methods comprise expanding T cells in vitro. In some embodiments, methods comprise expanding T cells ex vivo.

In some embodiments, administration of the polypeptide molecule or a pharmaceutical composition thereof to a subject results in an increased percentage or number of effector T cells in the subject. In some embodiments, the percentage or number of effector T cells is increased by at least about 10%, at least about 20%, at least about 30%, at least about 40%, at least about 50%, at least about 60%, at least about 70%, at least about 80%, at least about 90%, or at least about 100%, including any range or subrange therebetween, in the subject. In some embodiments, the percentage or number of effector T cells is increased by at least about 2-fold, at least about 3-fold, at least about 4-fold, at least about 5-fold, at least about 6-fold, at least about 7-fold, at least about 8-fold, at least about 9-fold, at least about 10-fold, at least about 25-fold, at least about 50-fold, or at least about 100-fold, including any range or subrange therebetween, in the subject. In some embodiments, the effector T cells are effector memory T cells (T_{EM}). In some embodiments, the effector T cells are T effector cells re-expressing CD45RA (T_{EMRA}).

In some embodiments, administration of the polypeptide molecule or a pharmaceutical composition thereof to a subject results in an increased percentage or number of memory T cells in the subject. In some embodiments, the percentage or number of memory T cells is increased by at least about 10%, at least about 20%, at least about 30%, at least about 40%, at least about 50%, at least about 60%, at least about 70%, at least about 80%, at least about 90%, or at least about 100%, including any range or subrange therebetween, in the subject. In some embodiments, the percentage or number of memory T cells is increased by at least about 2-fold, at least about 3-fold, at least about 4-fold, at least about 5-fold, at least about 6-fold, at least about 7-fold, at least about 8-fold, at least about 9-fold, at least about 10-fold, at least about 25-fold, at least about 50-fold, or at least about 100-fold, including any range or subrange therebetween, in the subject. In some embodiments, the memory T cells are central memory T cells (T_{CM}). In some embodiments, the memory T cells are effector memory T cells (T_{EM}). In some embodiments, the memory T cells are T memory stem cells (T_{SCM}).

In some embodiments, administration of the polypeptide molecule or a pharmaceutical composition thereof to a subject results in an increased percentage or number of CD8+ T cells in the subject. In some embodiments, the percentage or number of CD8+ T cells is increased by at least about 10%, at least about 20%, at least about 30%, at least about 40%, at least about 50%, at least about 60%, at least about 70%, at least about 80%, at least about 90%, or at least about 100%, including any range or subrange therebetween, in the subject. In some embodiments, the percentage or number of CD8+ T cells is increased by at least about 2-fold, at least about 3-fold, at least about 4-fold, at least about 5-fold, at least about 6-fold, at least about 7-fold, at least about 8-fold, at least about 9-fold, at least about 10-fold, at least about 25-fold, at least about 50-fold, or at least about 100-fold, including any range or subrange therebetween, in the subject.

In some embodiments, administration of the polypeptide molecule or a pharmaceutical composition thereof to a subject results in an increased percentage or number of CD4+ T cells in the subject. In some embodiments, the percentage or number of CD4+ T cells is increased by at least about 10%, at least about 20%, at least about 30%, at least about 40%, at least about 50%, at least about 60%, at least about 70%, at least about 80%, at least about 90%, or at least about 100%, including any range or subrange therebetween, in the subject. In some embodiments, the percentage or number of CD4+ T cells is increased by at least about 2-fold, at least about 3-fold, at least about 4-fold, at least about 5-fold, at least about 6-fold, at least about 7-fold, at least about 8-fold, at least about 9-fold, at least about 10-fold, at least about 25-fold, at least about 50-fold, or at least about 100-fold, including any range or subrange therebetween, in the subject.

In some embodiments, administration of the polypeptide molecule or a pharmaceutical composition thereof to a subject results in an increased percentage or number of regulatory T cells in the subject. In some embodiments, the percentage or number of regulatory T cells is increased by at least about 10%, at least about 20%, at least about 30%, at least about 40%, at least about 50%, at least about 60%, at least about 70%, at least about 80%, at least about 90%, or at least about 100%, including any range or subrange therebetween, in the subject. In some embodiments, the percentage or number of regulatory T cells is increased by at least about 2-fold, at least about 3-fold, at least about 4-fold, at least about 5-fold, at least about 6-fold, at least about 7-fold, at least about 8-fold, at least about 9-fold, at least about 10-fold, at least about 25-fold, at least about 50-fold, or at least about 100-fold, including any range or subrange therebetween, in the subject. In some embodiments, the regulatory T cells are CD4+ regulatory T cells.

In some embodiments, administration of the polypeptide molecule or a pharmaceutical composition thereof to a subject results in a decreased percentage or number of regulatory T cells in the subject. In some embodiments, the percentage or number of regulatory T cells is increased by at least about 10%, at least about 20%, at least about 30%, at least about 40%, at least about 50%, at least about 60%, at least about 70%, at least about 80%, at least about 90%, or at least about 100%, including any range or subrange therebetween, in the subject. In some embodiments, the regulatory T cells are CD4+ regulatory T cells.

In some embodiments, administration of the polypeptide molecule or a pharmaceutical composition thereof to a subject results in an increased ratio of CD8+ T cells to regulatory T cells in the subject. In some embodiments, the ratio of CD8+ T cells to regulatory T cells is increased by about 2 to about 4, about 2 to about 6, about 2 to about 10, about 2 to about 14, about 2 to about 18, about 2 to about 22, about 2 to about 26, about 2 to about 30, about 4 to about 6, about 4 to about 10, about 4 to about 14, about 4 to about 18, about 4 to about 22, about 4 to about 26, about 4 to about 30, about 6 to about 10, about 6 to about 14, about 6 to about 18, about 6 to about 22, about 6 to about 26, about 6 to about 30, about 10 to about 14, about 10 to about 18, about 10 to about 22, about 10 to about 26, about 10 to about 30, about 14 to about 18, about 14 to about 22, about 14 to about 26, about 14 to about 30, about 18 to about 22, about 18 to about 26, about 18 to about 30, about 22 to about 26, about 22 to about 30, about 26 to about 30, or any range or subrange in between, following administration of the polypeptide molecule or pharmaceutical composition thereof to the subject. In some embodiments, the ratio of CD8+ T cells to regulatory T cells is about 2, about 4, about 6, about 10, about 14, about 18, about 22, about 26, or about 30, including any range or subrange therebetween, following administration of the polypeptide molecule or pharmaceutical composition thereof to the subject. In some embodiments, the ratio of CD8+ T cells to regulatory T cells is increased by at least about 2-fold, at least about 3-fold, at least about 4-fold, at least about 5-fold, at least about 8-fold, at least about 10-fold, at least about 12-fold, at least about 15-fold, at least about 18-fold, at least about 20-fold, at least about 22-fold, at least about 25-fold, or at least about 30-fold, including any range or subrange therebetween, in the subject.

In some embodiments, administration of the polypeptide molecule or a pharmaceutical composition thereof to a subject results in an increased ratio of CD4+(non-Treg) T cells to regulatory T cells in the subject. In some embodiments, the ratio of CD4+(non-Treg) T cells to regulatory T cells is increased by about 2 to about 4, about 2 to about 6, about 2 to about 10, about 2 to about 14, about 2 to about 18, about 2 to about 22, about 2 to about 26, about 2 to about 30, about 4 to about 6, about 4 to about 10, about 4 to about 14, about 4 to about 18, about 4 to about 22, about 4 to about 26, about 4 to about 30, about 6 to about 10, about 6 to about 14, about 6 to about 18, about 6 to about 22, about 6 to about 26, about 6 to about 30, about 10 to about 14, about 10 to about 18, about 10 to about 22, about 10 to about 26, about 10 to about 30, about 14 to about 18, about 14 to about 22, about 14 to about 26, about 14 to about 30, about 18 to about 22, about 18 to about 26, about 18 to about 30, about 22 to about 26, about 22 to about 30, about 26 to about 30, or any range or subrange in between, following administration of the polypeptide molecule or pharmaceutical composition thereof to the subject. In some embodiments, the ratio of CD8+ T cells to regulatory T cells is about 2, about 4, about 6, about 10, about 14, about 18, about 22, about 26, or about 30, including any range or subrange therebetween, following administration of the polypeptide molecule or pharmaceutical composition thereof to the subject. In some embodiments, the ratio of CD4+(non-Treg) T cells to regulatory T cells is increased by at least about 2-fold, at least about 3-fold, at least about 4-fold, at least about 5-fold, at least about 8-fold, at least about 10-fold, at least about 12-fold, at least about 15-fold, at least about 18-fold, at least about 20-fold, at least about 22-fold, at least about 25-fold, or at least about 30-fold, including any range or subrange therebetween, in the subject. In some embodiments, the regulatory T cells are CD4+ regulatory T cells.

In some embodiments, administration of the polypeptide molecule or a pharmaceutical composition thereof to a subject results in an increase in T cell cytotoxicity in the subject. In some embodiments, T cell cytotoxicity is increased by at least about 10%, at least about 20%, at least about 30%, at least about 40%, at least about 50%, at least about 60%, at least about 70%, at least about 80%, at least about 90%, or at least about 100%, including any range or subrange therebetween, in the subject. In some embodiments, T cell cytotoxicity is increased by at least about 2-fold, at least about 3-fold, at least about 4-fold, at least about 5-fold, at least about 6-fold, at least about 7-fold, at least about 8-fold, at least about 9-fold, at least about 10-fold, at least about 25-fold, at least about 50-fold, or at least about 100-fold, including any range or subrange therebetween, in the subject. As used herein, the phrase “increase in T cell cytotoxicity” refers to an increase killing in target cells, an increase in expression of cytotoxic molecules, an increase in degranulation of cytotoxic T cells, an increase in proliferation of cytotoxic T cells, enhanced cytokine production by cytotoxic T cells, or increased survival of cytotoxic T cells. In some embodiments, the cytotoxic T cells are CD8+ cytotoxic T cells.

In some embodiments, administration of the polypeptide molecule or a pharmaceutical composition thereof to a subject results in reduced cytokine release in the subject when compared to a subject administered a polypeptide molecule comprising a CD3-binding domain. In some embodiments, the cytokine release is reduced by at least about 20%, at least about 30%, at least about 40%, at least about 50%, at least about 60%, at least about 70%, at least about 80%, at least about 90%, or at least about 100%, including any range or subrange therebetween, in the subject. In some embodiments, the cytokine release is reduced at least about 2-fold, at least about 3-fold, at least about 4 time, at least about 5-fold, at least about 6-fold, at least about 8-fold, at least about 10-fold, at least about 12-fold, at least about 14-fold, at least about 16-fold, at least about 18-fold, at least about 20-fold, at least about 22-fold, at least about 24-fold, at least about 25-fold, at least bout 50-fold, at least about 100-fold, or any values or subvalue in between. In some embodiments, the cytokine is interferon-gamma (IFNγ). In some embodiments, the cytokine is tumor necrosis factor (TNF). In some embodiments, the cytokine is interleukin-6 (IL-6). In some embodiments, the cytokine is interleukin-2 (IL-2). In some embodiments, the cytokine is interleukin-10 (IL-0).

Also provided herein are methods of depleting tumor cells in a subject having a disease, disorder, or condition related to the tumor cells (e.g., PSMA+ tumor cells or CD19+ tumor cells). In some embodiments, the method of depleting tumor cells in a subject in need thereof comprises administering a therapeutically effective amount of the polypeptide molecule or pharmaceutical composition thereof to the subject. In some embodiments, the method decreases the number of tumor cells by at least about 50%, at least about 55%, at least about 60%, at least about 65%, at least about 70%, at least about 75%, at least about 80%, at least about 85%, at least about 90%, at least about 95%, at least about 98%, at least about 99%, or at least about 100%, including any range or subrange therebetween, in the subject.

In some embodiments, the method depletes autoreactive cells in a subject having a disease, disorder, or condition related to autoreactive cells by administering a therapeutically effective amount of the polypeptide molecule or pharmaceutical composition thereof to the subject. In some embodiments, the method decreases the number of autoreactive cells by at least about 50%, at least about 55%, at least about 60%, at least about 65%, at least about 70%, at least about 75%, at least about 80%, at least about 85%, at least about 90%, at least about 95%, at least about 98%, at least about 99%, or at least about 100%, including any range or subrange therebetween, in the subject. In some embodiments, the autoreactive cells are CD19+ B cells.

In some embodiments, the method depletes B cells in a subject having a disease, disorder, or condition related to B cells by administering a therapeutically effective amount of the polypeptide molecule or pharmaceutical composition thereof to the subject. In some embodiments, the method decreases the number of B cells by at least about 50%, at least about 55%, at least about 60%, at least about 65%, at least about 70%, at least about 75%, at least about 80%, at least about 85%, at least about 90%, at least about 95%, at least about 98%, at least about 99%, or at least about 100%, including any range or subrange therebetween, in the subject.

### Numbered Embodiments

### Section 1.

Embodiment 1. A polypeptide molecule comprising an anti-TCRβV-binding domain, wherein the binding comprises:
a Kabat HCDR1 that comprises an amino acid sequence selected from any one of: TYYIH (SEQ ID NO: 112), LYYIH (SEQ ID NO: 136), TWYIH (SEQ ID NO: 145), and LWYIH (SEQ ID NO: 147);
a Kabat HCDR2 that comprises an amino acid sequence selected from any one of: WFFPGSGNIKYNEKFKG (SEQ ID NO: 69) and WFFPRSGNIKYNEKFKG (SEQ ID NO: 142)
a Kabat HCDR3 that comprises an amino acid sequence of SYYSFDVLDY (SEQ ID NO: 137)
a Kabat LCDR1 that comprises an amino acid sequence selected from any one of: KASQNVGINVV (SEQ ID NO: 71) and KASQNVGIRVV (SEQ ID NO: 134);
a Kabat LCDR2 that comprises an amino acid sequence selected from any one of: SSSHRYS (SEQ ID NO: 72), SSSHRYK (SEQ ID NO: 128), and SSSHRYR (SEQ ID NO: 144);
a Kabat LCDR3 that comprises an amino acid sequence selected from any one of: QQFKSYPIT (SEQ ID NO: 135) and QQFKFYPIT (SEQ ID NO: 141);
wherein each of the HCDR1, HCDR2, HCDR3, LCDR1, LCDR2, and/or LCDR3 independently comprise 0, 1, 2, or 3 amino acid mutations.

Embodiment 2. A polypeptide molecule comprising an anti-TCRβV binding domain, wherein the binding domain comprises:
(i) the Kabat HCDR1, the HCDR2, and the HCDR3 comprising the amino acid sequences of SEQ ID NOs: 112, 142, and 137, respectively, and the LCDR1, the LCDR2, and the LCDR3 comprising the amino acid sequences of SEQ ID NOs: 134, 72, and 141, respectively;
(ii) the Kabat HCDR1, the HCDR2, and the HCDR3 comprising the amino acid sequences of SEQ ID NOs: 136, 69, and 137, respectively, and the Kabat LCDR1, the LCDR2, and the LCDR3 comprising the amino acid sequences of SEQ ID NOs: 134, 72, and 135, respectively;
(iii) the Kabat HCDR1, the HCDR2, and the HCDR3 comprising the amino acid sequences of SEQ ID NOs: 112, 69, and 137, respectively, and the Kabat LCDR1, the LCDR2, and the LCDR3 comprising the amino acid sequences of SEQ ID NOs: 134, 144, and 135, respectively;
(iv) the Kabat HCDR1, the HCDR2, and the HCDR3 comprising the amino acid sequences of SEQ ID NOs: 112, 142, and 137, respectively, and the Kabat LCDR1, the LCDR2, and the LCDR3 comprising the amino acid sequences of SEQ ID NOs: 134, 72, and 135, respectively;
(v) the Kabat HCDR1, the HCDR2, and the HCDR3 comprising the amino acid sequences of SEQ ID NOs: 147, 69, and 137, respectively, and the Kabat LCDR1, the LCDR2, and the LCDR3 comprising the amino acid sequences of SEQ ID NOs: 134, 72, and 135, respectively;
(vi) the Kabat HCDR1, the HCDR2, and the HCDR3 comprising the amino acid sequences of SEQ ID NOs: 147, 142, and 137, respectively, and the Kabat LCDR1, the LCDR2, and the LCDR3 comprising the amino acid sequences of SEQ ID NOs: 71, 128, and 135, respectively;
(vii) the Kabat HCDR1, the HCDR2, and the HCDR3 comprising the amino acid sequences of SEQ ID NOs: 136, 142, and 137, respectively, and the Kabat LCDR1, the LCDR2, and the LCDR3 comprising the amino acid sequences of SEQ ID NOs: 134, 128, and 135, respectively;
(viii) the Kabat HCDR1, the HCDR2, and the HCDR3 comprising the amino acid sequences of SEQ ID NOs: 112, 142, and 137, respectively, and the Kabat LCDR1, the LCDR2, and the LCDR3 comprising the amino acid sequences of SEQ ID NOs: 71, 144, and 135, respectively;
(ix) the Kabat HCDR1, the HCDR2, and the HCDR3 comprising the amino acid sequences of SEQ ID NOs: 112, 142, and 137, respectively, and the Kabat LCDR1, the LCDR2, and the LCDR3 comprising the amino acid sequences of SEQ ID NOs: 134, 144, and 135, respectively;
(x) the Kabat HCDR1, the HCDR2, and the HCDR3 comprising the amino acid sequences of SEQ ID NOs: 136, 142, and 137, respectively, and the Kabat LCDR1, the LCDR2, and the LCDR3 comprising the amino acid sequences of SEQ ID NOs: 134, 144, and 135, respectively;
(xi) the Kabat HCDR1, the HCDR2, and the HCDR3 comprising the amino acid sequences of SEQ ID NOs: 145, 69, and 137, respectively, and the Kabat LCDR1, the LCDR2, and the LCDR3 comprising the amino acid sequences of SEQ ID NOs: 134, 128, and 141, respectively;
(xii) the Kabat HCDR1, the HCDR2, and the HCDR3 comprising the amino acid sequences of SEQ ID NOs: 147, 69, and 137, respectively, and the Kabat LCDR1, the LCDR2, and the LCDR3 comprising the amino acid sequences of SEQ ID NOs: 71, 144, and 141, respectively;
(xiii) the Kabat HCDR1, the HCDR2, and the HCDR3 comprising the amino acid sequences of SEQ ID NOs: 147, 69, and 137, respectively, and the Kabat LCDR1, the LCDR2, and the LCDR3 comprising the amino acid sequences of SEQ ID NOs: 134, 72, and 135, respectively;
(xiv) the Kabat HCDR1, the HCDR2, and the HCDR3 comprising the amino acid sequences of SEQ ID NOs: 112, 142, and 137, respectively, and the Kabat LCDR1, the LCDR2, and the LCDR3 comprising the amino acid sequences of SEQ ID NOs: 134, 128, and 141, respectively;
(xv) the Kabat HCDR1, the HCDR2, and the HCDR3 comprising the amino acid sequences of SEQ ID NOs: 136, 69, and 137, respectively, and the Kabat LCDR1, the LCDR2, and the LCDR3 comprising the amino acid sequences of SEQ ID NOs: 134, 128, and 141, respectively;
(xvi) the Kabat HCDR1, the HCDR2, and the HCDR3 comprising the amino acid sequences of SEQ ID NOs: 147, 142, and 137, respectively, and the Kabat LCDR1, the LCDR2, and the LCDR3 comprising the amino acid sequences of SEQ ID NOs: 134, 144, and 135, respectively;
(xvii) the Kabat HCDR1, the HCDR2, and the HCDR3 comprising the amino acid sequences of SEQ ID NOs: 136, 142, and 137, respectively, and the Kabat LCDR1, the LCDR2, and the LCDR3 comprising the amino acid sequences of SEQ ID NOs: 71, 144, and 135, respectively; or
(xviii) the Kabat HCDR1, the HCDR2, and the HCDR3 comprising the amino acid sequences of SEQ ID NOs: 147, 142, and 137, respectively, and the Kabat LCDR1, the LCDR2, and the LCDR3 comprising the amino acid sequences of SEQ ID NOs: 134, 144, and 141, respectively.

Embodiment 3. A polypeptide molecule comprising an anti-TCRβV binding domain, wherein the binding domain comprises:
a Kabat HCDR1 that comprises the amino acid sequence of TYYIH (SEQ ID NO: 112); a Kabat HCDR2 that comprises the amino acid sequence of WFFPRSGNIKYNEKFKG (SEQ ID NO: 142); a Kabat HCDR3 that comprises the amino acid sequence of SYYSFDVLDY (SEQ ID NO: 137);
a Kabat LCDR1 that comprises the amino acid sequence of KASQNVGIRVV (SEQ ID NO: 134); a Kabat LCDR2 that comprises the amino acid sequence of SSSHRYS (SEQ ID NO: 72); and
a Kabat LCDR3 that comprises the amino acid sequence of QQFKFYPIT (SEQ ID NO: 141).

Embodiment 4. A polypeptide molecule comprising an anti-TCRβV binding domain, wherein the binding domain comprises:
a Kabat HCDR1 that comprises the amino acid sequence of TYYIH (SEQ ID NO: 112); a Kabat HCDR2 that comprises the amino acid sequence of WFFPGSGNIKYNEKFKG (SEQ ID NO: 69); a Kabat HCDR3 that comprises the amino acid sequence of SYYSFDVLDY (SEQ ID NO: 137);
a Kabat LCDR1 that comprises the amino acid sequence of KASQNVGIRVV (SEQ ID NO: 134); a Kabat LCDR2 that comprises the amino acid sequence of SSSHRYR (SEQ ID NO: 144); and
a Kabat LCDR3 that comprises the amino acid sequence of QQFKSYPIT (SEQ ID NO: 135).

Embodiment 5. A polypeptide molecule comprising an anti-TCRβV binding domain, wherein the binding domain comprises:
a Kabat HCDR1 that comprises the amino acid sequence of LYYIH (SEQ ID NO: 136); a Kabat HCDR2 that comprises the amino acid sequence of WFFPGSGNIKYNEKFKG (SEQ ID NO: 69); a Kabat HCDR3 that comprises the amino acid sequence of SYYSFDVLDY (SEQ ID NO: 137);
a Kabat LCDR1 that comprises the amino acid sequence of KASQNVGIRVV (SEQ ID NO: 134); a Kabat LCDR2 that comprises the amino acid sequence of SSSHRYS (SEQ ID NO: 72); and
a Kabat LCDR3 that comprises the amino acid sequence of QQFKSYPIT (SEQ ID NO: 135).

Embodiment 6. A polypeptide molecule comprising an anti-TCRβV binding domain, wherein the binding domain comprises:
a Kabat HCDR1 that comprises the amino acid sequence of LWYIH (SEQ ID NO: 147); a Kabat HCDR2 that comprises the amino acid sequence of WFFPGSGNIKYNEKFKG (SEQ ID NO: 69); a Kabat HCDR3 that comprises the amino acid sequence of SYYSFDVLDY (SEQ ID NO: 137);
a Kabat LCDR1 that comprises the amino acid sequence of KASQNVGIRVV (SEQ ID NO: 134); a Kabat LCDR2 that comprises the amino acid sequence of SSSHRYS (SEQ ID NO: 72); and
a Kabat LCDR3 that comprises the amino acid sequence of QQFKSYPIT (SEQ ID NO: 135).

Embodiment 7. A polypeptide molecule comprising an anti-TCRβV binding domain, wherein the binding domain comprises:
a Kabat HCDR1 that comprises the amino acid sequence of LWYIH (SEQ ID NO: 147); a Kabat HCDR2 that comprises the amino acid sequence of WFFPRSGNIKYNEKFKG (SEQ ID NO: 142); a Kabat HCDR3 that comprises the amino acid sequence of SYYSFDVLDY (SEQ ID NO: 137);
a Kabat LCDR1 that comprises the amino acid sequence of KASQNVGINVV (SEQ ID NO: 71); a Kabat LCDR2 that comprises the amino acid sequence of (SEQ ID NO: 128); and a Kabat LCDR3 that comprises the amino acid sequence of QQFKSYPIT (SEQ ID NO: 135).

Embodiment 8. A polypeptide molecule comprising an anti-TCRβV binding domain, wherein the binding domain comprises:
a Kabat heavy chain CDR1 (HCDR1) that comprises the amino acid sequence of TYYIH (SEQ ID NO: 112); a Kabat heavy chain CDR2 (HCDR2) that comprises the amino acid sequence of WFFPRSGNIKYNEKFKG (SEQ ID NO: 142); a Kabat heavy chain CDR3 (HCDR3) that comprises the amino acid sequence of SYYSFDVLDY (SEQ ID NO: 137);
a Kabat light chain CDR1 (LCDR1) that comprises the amino acid sequence of KASQNVGIRVV (SEQ ID NO: 134); a Kabat light chain CDR2 (LCDR2) that comprises the amino acid sequence of SSSHRYS (SEQ ID NO: 72); and a Kabat light chain CDR3 (LCDR3) that comprises the amino acid sequence of QQFKSYPIT (SEQ ID NO: 135).

Embodiment 9. A polypeptide molecule comprising an anti-TCRβV binding domain, wherein the binding domain comprises:
a Kabat HCDR1 that comprises the amino acid sequence of LYYIH (SEQ ID NO: 136); a Kabat HCDR2 that comprises the amino acid sequence of WFFPRSGNIKYNEKFKG (SEQ ID NO: 142); a Kabat HCDR3 that comprises the amino acid sequence of SYYSFDVLDY (SEQ ID NO: 137);
a Kabat LCDR1 that comprises the amino acid sequence of (SEQ ID NO: 134); a Kabat LCDR2 that comprises the amino acid sequence of (SEQ ID NO: 128); and a Kabat LCDR3 that comprises the amino acid sequence of QQFKSYPIT (SEQ ID NO: 135).

Embodiment 10. A polypeptide molecule comprising an anti-TCRβV binding domain, wherein the binding domain comprises:
a Kabat HCDR1 that comprises the amino acid sequence of TYYIH (SEQ ID NO: 112); a Kabat HCDR2 that comprises the amino acid sequence of WFFPRSGNIKYNEKFKG (SEQ ID NO: 142); a Kabat HCDR3 that comprises the amino acid sequence of SYYSFDVLDY (SEQ ID NO: 137);
a Kabat LCDR1 that comprises the amino acid sequence of KASQNVGINVV (SEQ ID NO: 71); a Kabat LCDR2 that comprises the amino acid sequence of SSSHRYR (SEQ ID NO: 144); and
a Kabat LCDR3 that comprises the amino acid sequence of QQFKSYPIT (SEQ ID NO: 135).

Embodiment 11. A polypeptide molecule comprising an anti-TCRβV-binding domain, wherein the binding domain comprises:
an IMGT HCDR1 that comprises an amino acid sequence selected from any one of: GYSFTTYY (SEQ ID NO: 114), GYSFTLYY (SEQ ID NO: 139), GYSFTTWY (SEQ ID NO: 146), and GYSFTLWY (SEQ ID NO: 148);
an IMGT HCDR2 that comprises an amino acid sequence selected from any one of: FFPGSGNI (SEQ ID NO: 115) and FFPRSGNI (SEQ ID NO: 143);
an IMGT HCDR3 that comprises an amino acid sequence of AASYYSFDVLDY (SEQ ID NO: 140);
an IMGT LCDR1 that comprises an amino acid sequence selected from any one of: QNVGIN (SEQ ID NO: 113) and QNVGIR (SEQ ID NO: 138);
an IMGT LCDR2 that comprises an amino acid sequence of SSS;
an IMGT LCDR3 that comprises an amino acid sequence selected from any one of: QQFKSYPIT (SEQ ID NO: 135) and QQFKFYPIT (SEQ ID NO: 141);
wherein each of the HCDR1, HCDR2, HCDR3, LCDR1, LCDR2, and/or LCDR3 independently comprise 0, 1, 2, or 3 amino acid mutations.

Embodiment 12. A polypeptide molecule comprising an anti-TCRβV-binding domain, wherein the binding domain comprises:
(i) the IMGT HCDR1, the HCDR2, and the HCDR3 comprising the amino acid sequences of SEQ ID NOs: 114, 143, and 140, respectively, and the IMGT LCDR1, the LCDR2, and the LCDR3 comprising the amino acid sequences of SEQ ID NOs: 138, SSS, and 141, respectively;
(ii) the IMGT HCDR1, the HCDR2, and the HCDR3 comprising the amino acid sequences of SEQ ID NOs: 139, 115, and 140, respectively, and the IMGT LCDR1, the LCDR2, and the LCDR3 comprising the amino acid sequences of SEQ ID NOs: 138, SSS, and 135, respectively;
(iii) the IMGT HCDR1, the HCDR2, and the HCDR3 comprising the amino acid sequences of SEQ ID NOs: 114, 115, and 140, respectively, and the IMGT LCDR1, the LCDR2, and the LCDR3 comprising the amino acid sequences of SEQ ID NOs: 138, SSS, and 135, respectively;
(iv) the IMGT HCDR1, the HCDR2, and the HCDR3 comprising the amino acid sequences of SEQ ID NOs: 114, 143, and 140, respectively, and the IMGT LCDR1, the LCDR2, and the LCDR3 comprising the amino acid sequences of SEQ ID NOs: 138, SSS, and 135, respectively;
(v) the IMGT HCDR1, the HCDR2, and the HCDR3 comprising the amino acid sequences of SEQ ID NOs: 148, 115, and 140, respectively, and the IMGT LCDR1, the LCDR2, and the LCDR3 comprising the amino acid sequences of SEQ ID NOs: 138, SSS, and 135, respectively;
(vi) the IMGT HCDR1, the HCDR2, and the HCDR3 comprising the amino acid sequences of SEQ ID NOs: 148, 143, and 140, respectively, and the IMGT LCDR1, the LCDR2, and the LCDR3 comprising the amino acid sequences of SEQ ID NOs: 113, SSS, and 135, respectively;
(vii) the IMGT HCDR1, the HCDR2, and the HCDR3 comprising the amino acid sequences of SEQ ID NOs: 139, 143, and 140, respectively, and the IMGT LCDR1, the LCDR2, and the LCDR3 comprising the amino acid sequences of SEQ ID NOs: 138, SSS, and 135, respectively;
(viii) the IMGT HCDR1, the HCDR2, and the HCDR3 comprising the amino acid sequences of SEQ ID NOs: 114, 143, and 140, respectively, and the IMGT LCDR1, the LCDR2, and the LCDR3 comprising the amino acid sequences of SEQ ID NOs: 113, SSS, and 135, respectively;
(ix) the IMGT HCDR1, the HCDR2, and the HCDR3 comprising the amino acid sequences of SEQ ID NOs: 114, 143, and 140, respectively, and the IMGT LCDR1, the LCDR2, and the LCDR3 comprising the amino acid sequences of SEQ ID NOs: 138, SSS, and 135, respectively;
(x) the IMGT HCDR1, the HCDR2, and the HCDR3 comprising the amino acid sequences of SEQ ID NOs: 139, 143, and 140, respectively, and the IMGT LCDR1, the LCDR2, and the LCDR3 comprising the amino acid sequences of SEQ ID NOs: 138, SSS, and 135, respectively;
(xi) the IMGT HCDR1, the HCDR2, and the HCDR3 comprising the amino acid sequences of SEQ ID NOs: 146, 115, and 140, respectively, and the IMGT LCDR1, the LCDR2, and the LCDR3 comprising the amino acid sequences of SEQ ID NOs: 138, SSS, and 141, respectively;
(xii) the IMGT HCDR1, the HCDR2, and the HCDR3 comprising the amino acid sequences of SEQ ID NOs: 148, 115, and 140, respectively, and the IMGT LCDR1, the LCDR2, and the LCDR3 comprising the amino acid sequences of SEQ ID NOs: 138, SSS, and 141, respectively;
(xiii) the IMGT HCDR1, the HCDR2, and the HCDR3 comprising the amino acid sequences of SEQ ID NOs: 148, 115, and 140, respectively, and the IMGT LCDR1, the LCDR2, and the LCDR3 comprising the amino acid sequences of SEQ ID NOs: 138, SSS, and 135, respectively;
(xiv) the IMGT HCDR1, the HCDR2, and the HCDR3 comprising the amino acid sequences of SEQ ID NOs: 114, 143, and 140, respectively, and the IMGT LCDR1, the LCDR2, and the LCDR3 comprising the amino acid sequences of SEQ ID NOs: 138, SSS, and 141, respectively;
(xv) the IMGT HCDR1, the HCDR2, and the HCDR3 comprising the amino acid sequences of SEQ ID NOs: 139, 115, and 140, respectively, and the IMGT LCDR1, the LCDR2, and the LCDR3 comprising the amino acid sequences of SEQ ID NOs: 138, SSS, and 141, respectively;
(xvi) the IMGT HCDR1, the HCDR2, and the HCDR3 comprising the amino acid sequences of SEQ ID NOs: 148, 143, and 140, respectively, and the IMGT LCDR1, the LCDR2, and the LCDR3 comprising the amino acid sequences of SEQ ID NOs: 138, SSS, and 135, respectively;
(xvii) the IMGT HCDR1, the HCDR2, and the HCDR3 comprising the amino acid sequences of SEQ ID NOs: 139, 143, and 140, respectively, and the IMGT LCDR1, the LCDR2, and the LCDR3 comprising the amino acid sequences of SEQ ID NOs: 138, SSS, and 135, respectively; or
(xviii) the IMGT HCDR1, the HCDR2, and the HCDR3 comprising the amino acid sequences of SEQ ID NOs: 148, 143, and 140, respectively, and the IMGT LCDR1, the LCDR2, and the LCDR3 comprising the amino acid sequences of SEQ ID NOs: 138, SSS, and 141, respectively.

Embodiment 13. A polypeptide molecule comprising an anti-TCRβV binding domain, wherein the binding domain comprises:
an IMGT HCDR1 that comprises the amino acid sequence of GYSFTTYY (SEQ ID NO: 114); an IMGT HCDR2 that comprises the amino acid sequence of FFPRSGNI (SEQ ID NO: 143);
an IMGT HCDR3 that comprises the amino acid sequence of AASYYSFDVLDY (SEQ ID NO: 140);
an IMGT LCDR1 that comprises the amino acid sequence of QNVGIR (SEQ ID NO: 138);
an IMGT LCDR2 that comprises the amino acid sequence of SSS; and an IMGT LCDR3 that comprises the amino acid sequence of QQFKFYPIT (SEQ ID NO: 141).

Embodiment 14. A polypeptide molecule comprising an anti-TCRβV binding domain, wherein the binding domain comprises:
an IMGT HCDR1 that comprises the amino acid sequence of GYSFTTYY (SEQ ID NO: 114); an IMGT HCDR2 that comprises the amino acid sequence of FFPGSGNI (SEQ ID NO: 115);
an IMGT HCDR3 that comprises the amino acid sequence of AASYYSFDVLDY (SEQ ID NO: 140);
an IMGT LCDR1 that comprises the amino acid sequence of QNVGIR (SEQ ID NO: 138);
an IMGT LCDR2 that comprises the amino acid sequence of SSS; and an IMGT LCDR3 that comprises the amino acid sequence of QQFKSYPIT (SEQ ID NO: 135).

Embodiment 15. A polypeptide molecule comprising an anti-TCRβV-binding domain, wherein the binding domain comprises:
an IMGT HCDR1 that comprises the amino acid sequence of GYSFTLYY (SEQ ID NO: 139); an IMGT HCDR2 that comprises the amino acid sequence of FFPGSGNI (SEQ ID NO: 115);
an IMGT HCDR3 that comprises the amino acid sequence of AASYYSFDVLDY (SEQ ID NO: 140);
an IMGT LCDR1 that comprises the amino acid sequence of QNVGIR (SEQ ID NO: 138);
an IMGT LCDR2 that comprises the amino acid sequence of SSS; and an IMGT LCDR3 that comprises the amino acid sequence of QQFKSYPIT (SEQ ID NO: 135).

Embodiment 16. A polypeptide molecule comprising an anti-TCRβV binding domain, wherein the binding domain comprises:
an IMGT HCDR1 that comprises the amino acid sequence of GYSFTLWY (SEQ ID NO: 148); an IMGT HCDR2 that comprises the amino acid sequence of FFPGSGNI (SEQ ID NO: 115); an IMGT HCDR3 that comprises the amino acid sequence of AASYYSFDVLDY (SEQ ID NO: 140);
an IMGT LCDR1 that comprises the amino acid sequence of QNVGIR (SEQ ID NO: 138);
an IMGT LCDR2 that comprises the amino acid sequence of SSS; and an IMGT LCDR3 that comprises the amino acid sequence of QQFKSYPIT (SEQ ID NO: 135).

Embodiment 17. A polypeptide molecule comprising an anti-TCRβV binding domain, wherein the binding domain comprises:
an IMGT HCDR1 that comprises the amino acid sequence of GYSFTLWY (SEQ ID NO: 148); an IMGT HCDR2 that comprises the amino acid sequence of FFPRSGNI (SEQ ID NO: 143); an IMGT HCDR3 that comprises the amino acid sequence of AASYYSFDVLDY (SEQ ID NO: 140);
an IMGT LCDR1 that comprises the amino acid sequence of QNVGIN (SEQ ID NO: 113);
an IMGT LCDR2 that comprises the amino acid sequence of SSS; and an IMGT LCDR3 that comprises the amino acid sequence of QQFKSYPIT (SEQ ID NO: 135).

Embodiment 18. A polypeptide molecule comprising an anti-TCRβV-binding domain, wherein the binding domain comprises:
an IMGT HCDR1 that comprises the amino acid sequence of GYSFTTYY (SEQ ID NO: 114); an IMGT HCDR2 that comprises the amino acid sequence of FFPRSGNI (SEQ ID NO: 143);
an IMGT HCDR3 that comprises the amino acid sequence of AASYYSFDVLDY (SEQ ID NO: 140);
an IMGT LCDR1 that comprises the amino acid sequence of QNVGIR (SEQ ID NO: 138); an IMGT LCDR2 that comprises the amino acid sequence of SSS; and an IMGT LCDR3 that comprises the amino acid sequence of QQFKSYPIT (SEQ ID NO: 135).

Embodiment 19. A polypeptide molecule comprising an anti-TCRβV binding domain, wherein the binding domain comprises:
an IMGT HCDR1 that comprises the amino acid sequence of GYSFTLYY (SEQ ID NO: 139); an IMGT HCDR2 that comprises the amino acid sequence of FFPRSGNI (SEQ ID NO: 143); an IMGT HCDR3 that comprises the amino acid sequence of AASYYSFDVLDY (SEQ ID NO: 140);
an IMGT LCDR1 that comprises the amino acid sequence of QNVGIR (SEQ ID NO: 138); an IMGT LCDR2 that comprises the amino acid sequence of SSS; and an IMGT LCDR3 that comprises the amino acid sequence of QQFKSYPIT (SEQ ID NO: 135).

Embodiment 20. A polypeptide molecule comprising an anti-TCRβV binding domain, wherein the binding domain comprises:
an IMGT HCDR1 that comprises the amino acid sequence of GYSFTTYY (SEQ ID NO: 114); an IMGT HCDR2 that comprises the amino acid sequence of FFPRSGNI (SEQ ID NO: 143);
an IMGT HCDR3 that comprises the amino acid sequence of AASYYSFDVLDY (SEQ ID NO: 140);
an IMGT LCDR1 that comprises the amino acid sequence of QNVGIN (SEQ ID NO: 113);
an IMGT LCDR2 that comprises the amino acid sequence of SSS; and an IMGT LCDR3 that comprises the amino acid sequence of QQFKSYPIT (SEQ ID NO: 135).

Embodiment 21. A polypeptide molecule comprising an anti-TCRβV-binding domain, wherein the binding domain comprises:
a Kabat HCDR1 that comprises an amino acid sequence selected from any one of: NFGMH (SEQ ID NO: 1160); IFGMH (SEQ ID NO: 1162); RTGMH (SEQ ID NO: 1163); IKGMH (SEQ ID NO: 1164); NKGMH (SEQ ID NO: 1165); RFGMH (SEQ ID NO: 1166); RKGMH (SEQ ID NO: 1167); ITGMH (SEQ ID NO: 1168); IYGMH (SEQ ID NO: 1169); and NTGMH (SEQ ID NO: 1170);
a Kabat HCDR2 that comprises an amino acid sequence selected from any one of: YISSGSSTIYYADTLKG (SEQ ID NO: 1171), YISSTSSTILYADTLKG (SEQ ID NO: 1173), YISSSSSTILYADTLKG (SEQ ID NO: 1174), YISSSSSTIYYADTLKG (SEQ ID NO: 1175), YISSWSSTILYADTLKG (SEQ ID NO: 1176), YISSWSSTIYYADTLKG (SEQ ID NO: 1177), YISSTSSTIYYADTLKG (SEQ ID NO: 1178), and YISSGSSTILYADTLKG (SEQ ID NO: 1179);
a Kabat HCDR3 that comprises an amino acid sequence of RGEGAMDY (SEQ ID NO: 1180) and RGAGAMDY (SEQ ID NO: 1181);
a Kabat LCDR1 that comprises an amino acid sequence selected from any one of: RASSSVNYIY (SEQ ID NO: 1148), RASRSVNYAY (SEQ ID NO: 1151), RASKSVNYAY (SEQ ID NO: 1152), RASKSVNYIY (SEQ ID NO: 1153), RASSSVNYAY (SEQ ID NO: 1154), and RASRSVNYIY (SEQ ID NO: 1155);
a Kabat LCDR2 that comprises an amino acid sequence of YTSNLAP (SEQ ID NO: 1156);
a Kabat LCDR3 that comprises an amino acid sequence selected from any one of: QQFTSSPFT (SEQ ID NO: 1157) and QQFTSSPFV (SEQ ID NO: 1159);
wherein each of the HCDR1, HCDR2, HCDR3, LCDR1, LCDR2, and/or LCDR3 independently comprise 0, 1, 2, or 3 amino acid mutations.

Embodiment 22. The polypeptide of embodiment 21, wherein the binding domain comprises:
(i) the Kabat HCDR1, the HCDR2, and the HCDR3 comprising the amino acid sequences of SEQ ID NOs: 1162, 1173, and 1180, respectively, and the Kabat LCDR1, the LCDR2, and the LCDR3 comprising the amino acid sequences of SEQ ID NOs: 1151, 1156, and 1159, respectively;
(ii) the Kabat HCDR1, the HCDR2, and the HCDR3 comprising the amino acid sequences of SEQ ID NOs: 1162, 1173, and 1180, respectively, and the Kabat LCDR1, the LCDR2, and the LCDR3 comprising the amino acid sequences of SEQ ID NOs: 1148, 1156, and 1159, respectively;
(iii) the Kabat HCDR1, the HCDR2, and the HCDR3 comprising the amino acid sequences of SEQ ID NOs: 1163, 1174, and 1180, respectively, and the Kabat LCDR1, the LCDR2, and the LCDR3 comprising the amino acid sequences of SEQ ID NOs: 1151, 1156, and 1159, respectively;
(iv) the Kabat HCDR1, the HCDR2, and the HCDR3 comprising the amino acid sequences of SEQ ID NOs: 1164, 1171, and 1181, respectively, and the Kabat LCDR1, the LCDR2, and the LCDR3 comprising the amino acid sequences of SEQ ID NOs: 1152, 1156, and 1159, respectively;
(v) the Kabat HCDR1, the HCDR2, and the HCDR3 comprising the amino acid sequences of SEQ ID NOs: 1165, 1175, and 1180, respectively, and the Kabat LCDR1, the LCDR2, and the LCDR3 comprising the amino acid sequences of SEQ ID NOs: 1153, 1156, and 1157, respectively;
(vi) the Kabat HCDR1, the HCDR2, and the HCDR3 comprising the amino acid sequences of SEQ ID NOs: 1160, 1176, and 1181, respectively, and the Kabat LCDR1, the LCDR2, and the LCDR3 comprising the amino acid sequences of SEQ ID NOs: 1154, 1156, and 1159, respectively;
(vii) the Kabat HCDR1, the HCDR2, and the HCDR3 comprising the amino acid sequences of SEQ ID NOs: 1162, 1177, and 1180, respectively, and the Kabat LCDR1, the LCDR2, and the LCDR3 comprising the amino acid sequences of SEQ ID NOs: 1151, 1156, and 1157, respectively;
(viii) the Kabat HCDR1, the HCDR2, and the HCDR3 comprising the amino acid sequences of SEQ ID NOs: 1166, 1176, and 1180, respectively, and the Kabat LCDR1, the LCDR2, and the LCDR3 comprising the amino acid sequences of SEQ ID NOs: 1153, 1156, and 1159, respectively;
(ix) the Kabat HCDR1, the HCDR2, and the HCDR3 comprising the amino acid sequences of SEQ ID NOs: 1167, 1173, and 1180, respectively, and the Kabat LCDR1, the LCDR2, and the LCDR3 comprising the amino acid sequences of SEQ ID NOs: 1155, 1156, and 1159, respectively;
(x) the Kabat HCDR1, the HCDR2, and the HCDR3 comprising the amino acid sequences of SEQ ID NOs: 1162, 1177, and 1181, respectively, and the Kabat LCDR1, the LCDR2, and the LCDR3 comprising the amino acid sequences of SEQ ID NOs: 1154, 1156, and 1159, respectively;
(xi) the Kabat HCDR1, the HCDR2, and the HCDR3 comprising the amino acid sequences of SEQ ID NOs: 1162, 1174, and 1181, respectively, and the Kabat LCDR1, the LCDR2, and the LCDR3 comprising the amino acid sequences of SEQ ID NOs: 1154, 1156, and 1159, respectively;
(xii) the Kabat HCDR1, the HCDR2, and the HCDR3 comprising the amino acid sequences of SEQ ID NOs: 1165, 1174, and 1181, respectively, and the Kabat LCDR1, the LCDR2, and the LCDR3 comprising the amino acid sequences of SEQ ID NOs: 1154, 1156, and 1157, respectively;
(xiii) the Kabat HCDR1, the HCDR2, and the HCDR3 comprising the amino acid sequences of SEQ ID NOs: 1166, 1178, and 1180, respectively, and the Kabat LCDR1, the LCDR2, and the LCDR3 comprising the amino acid sequences of SEQ ID NOs: 1151, 1156, and 1157, respectively;
(xiv) the Kabat HCDR1, the HCDR2, and the HCDR3 comprising the amino acid sequences of SEQ ID NOs: 1168, 1173, and 1181, respectively, and the Kabat LCDR1, the LCDR2, and the LCDR3 comprising the amino acid sequences of SEQ ID NOs: 1153, 1156, and 1159, respectively;
(xv) the Kabat HCDR1, the HCDR2, and the HCDR3 comprising the amino acid sequences of SEQ ID NOs: 1167, 1171, and 1181, respectively, and the Kabat LCDR1, the LCDR2, and the LCDR3 comprising the amino acid sequences of SEQ ID NOs: 1154, 1156, and 1157, respectively;
(xvi) the Kabat HCDR1, the HCDR2, and the HCDR3 comprising the amino acid sequences of SEQ ID NOs: 1168, 1177, and 1181, respectively, and the Kabat LCDR1, the LCDR2, and the LCDR3 comprising the amino acid sequences of SEQ ID NOs: 1153, 1156, and 1157, respectively;
(xvii) the Kabat HCDR1, the HCDR2, and the HCDR3 comprising the amino acid sequences of SEQ ID NOs: 1164, 1174, and 1181, respectively, and the Kabat LCDR1, the LCDR2, and the LCDR3 comprising the amino acid sequences of SEQ ID NOs: 1153, 1156, and 1159, respectively;
(xviii) the Kabat HCDR1, the HCDR2, and the HCDR3 comprising the amino acid sequences of SEQ ID NOs: 1167, 1177, and 1181, respectively, and the Kabat LCDR1, the LCDR2, and the LCDR3 comprising the amino acid sequences of SEQ ID NOs: 1154, 1156, and 1157, respectively;
(xix) the Kabat HCDR1, the HCDR2, and the HCDR3 comprising the amino acid sequences of SEQ ID NOs: 1162, 1173, and 1180, respectively, and the Kabat LCDR1, the LCDR2, and the LCDR3 comprising the amino acid sequences of SEQ ID NOs: 1151, 1156, and 1157, respectively;
(xx) the Kabat HCDR1, the HCDR2, and the HCDR3 comprising the amino acid sequences of SEQ ID NOs: 1169, 1173, and 1181, respectively, and the Kabat LCDR1, the LCDR2, and the LCDR3 comprising the amino acid sequences of SEQ ID NOs: 1154, 1156, and 1157, respectively;
(xxi) the Kabat HCDR1, the HCDR2, and the HCDR3 comprising the amino acid sequences of SEQ ID NOs: 1163, 1179, and 1180, respectively, and the Kabat LCDR1, the LCDR2, and the LCDR3 comprising the amino acid sequences of SEQ ID NOs: 1152, 1156, and 1159, respectively;
(xxii) the Kabat HCDR1, the HCDR2, and the HCDR3 comprising the amino acid sequences of SEQ ID NOs: 1170, 1173, and 1181, respectively, and the Kabat LCDR1, the LCDR2, and the LCDR3 comprising the amino acid sequences of SEQ ID NOs: 1151, 1156, and 1157, respectively;
(xxiii) the Kabat HCDR1, the HCDR2, and the HCDR3 comprising the amino acid sequences of SEQ ID NOs: 1165, 1179, and 1180, respectively, and the Kabat LCDR1, the LCDR2, and the LCDR3 comprising the amino acid sequences of SEQ ID NOs: 1152, 1156, and 1157, respectively;
(xxiv) the Kabat HCDR1, the HCDR2, and the HCDR3 comprising the amino acid sequences of SEQ ID NOs: 1168, 1173, and 1181, respectively, and the Kabat LCDR1, the LCDR2, and the LCDR3 comprising the amino acid sequences of SEQ ID NOs: 1155, 1156, and 1159, respectively;
(xxv) the Kabat HCDR1, the HCDR2, and the HCDR3 comprising the amino acid sequences of SEQ ID NOs: 1166, 1178, and 1180, respectively, and the Kabat LCDR1, the LCDR2, and the LCDR3 comprising the amino acid sequences of SEQ ID NOs: 1151, 1156, and 1159, respectively;
(xxvi) the Kabat HCDR1, the HCDR2, and the HCDR3 comprising the amino acid sequences of SEQ ID NOs: 1168, 1175, and 1181, respectively, and the Kabat LCDR1, the LCDR2, and the LCDR3 comprising the amino acid sequences of SEQ ID NOs: 1152, 1156, and 1159, respectively;
(xxvii) the Kabat HCDR1, the HCDR2, and the HCDR3 comprising the amino acid sequences of SEQ ID NOs: 1163, 1177, and 1180, respectively, and the Kabat LCDR1, the LCDR2, and the LCDR3 comprising the amino acid sequences of SEQ ID NOs: 1154, 1156, and 1159, respectively;
(xxviii) the Kabat HCDR1, the HCDR2, and the HCDR3 comprising the amino acid sequences of SEQ ID NOs: 1167, 1177, and 1181, respectively, and the Kabat LCDR1, the LCDR2, and the LCDR3 comprising the amino acid sequences of SEQ ID NOs: 1153, 1156, and 1159, respectively;
(xxix) the Kabat HCDR1, the HCDR2, and the HCDR3 comprising the amino acid sequences of SEQ ID NOs: 1160, 1177, and 1181, respectively, and the Kabat LCDR1, the LCDR2, and the LCDR3 comprising the amino acid sequences of SEQ ID NOs: 1153, 1156, and 1159, respectively;
(xxx) the Kabat HCDR1, the HCDR2, and the HCDR3 comprising the amino acid sequences of SEQ ID NOs: 1170, 1173, and 1180, respectively, and the Kabat LCDR1, the LCDR2, and the LCDR3 comprising the amino acid sequences of SEQ ID NOs: 1151, 1156, and 1159, respectively;
(xxxi) the Kabat HCDR1, the HCDR2, and the HCDR3 comprising the amino acid sequences of SEQ ID NOs: 1170, 1174, and 1181, respectively, and the Kabat LCDR1, the LCDR2, and the LCDR3 comprising the amino acid sequences of SEQ ID NOs: 1151, 1156, and 1159, respectively;
(xxxii) the Kabat HCDR1, the HCDR2, and the HCDR3 comprising the amino acid sequences of SEQ ID NOs: 1163, 1176, and 1181, respectively, and the Kabat LCDR1, the LCDR2, and the LCDR3 comprising the amino acid sequences of SEQ ID NOs: 1151, 1156, and 1159, respectively;
(xxxiii) the Kabat HCDR1, the HCDR2, and the HCDR3 comprising the amino acid sequences of SEQ ID NOs: 1165, 1176, and 1181, respectively, and the Kabat LCDR1, the LCDR2, and the LCDR3 comprising the amino acid sequences of SEQ ID NOs: 1154, 1156, and 1157, respectively;
(xxxiv) the Kabat HCDR1, the HCDR2, and the HCDR3 comprising the amino acid sequences of SEQ ID NOs: 1165, 1179, and 1181, respectively, and the Kabat LCDR1, the LCDR2, and the LCDR3 comprising the amino acid sequences of SEQ ID NOs: 1151, 1156, and 1159, respectively;
(xxxv) the Kabat HCDR1, the HCDR2, and the HCDR3 comprising the amino acid sequences of SEQ ID NOs: 1163, 1174, and 1180, respectively, and the Kabat LCDR1, the LCDR2, and the LCDR3 comprising the amino acid sequences of SEQ ID NOs: 1152, 1156, and 1159, respectively;
(xxxvi) the Kabat HCDR1, the HCDR2, and the HCDR3 comprising the amino acid sequences of SEQ ID NOs: 1170, 1175, and 1181, respectively, and the Kabat LCDR1, the LCDR2, and the LCDR3 comprising the amino acid sequences of SEQ ID NOs: 1151, 1156, and 1159, respectively; or
(xxxvii) the Kabat HCDR1, the HCDR2, and the HCDR3 comprising the amino acid sequences of SEQ ID NOs: 1168, 1174, and 1180, respectively, and the Kabat LCDR1, the LCDR2, and the LCDR3 comprising the amino acid sequences of SEQ ID NOs: 1155, 1156, and 1159, respectively.

Embodiment 23. A polypeptide molecule comprising an anti-TCRβV binding domain, wherein the binding domain comprises:
a Kabat HCDR1 that comprises the amino acid sequence of NTGMH (SEQ ID NO: 1170); a Kabat HCDR2 that comprises the amino acid sequence of YISSTSSTILYADTLKG (SEQ ID NO: 1173); a Kabat HCDR3 that comprises the amino acid sequence of RGAGAMDY (SEQ ID NO: 1181);
a Kabat LCDR1 that comprises the amino acid sequence of RASRSVNYAY (SEQ ID NO: 1151); a Kabat LCDR2 that comprises the amino acid sequence of YTSNLAP (SEQ ID NO: 1156); and a Kabat LCDR3 that comprises the amino acid sequence of QQFTSSPFT (SEQ ID NO: 1157).

Embodiment 24. A polypeptide molecule comprising an anti-TCRβV binding domain, wherein the binding domain comprises:
a Kabat HCDR1 that comprises the amino acid sequence of ITGMH (SEQ ID NO: 1168); a Kabat HCDR2 that comprises the amino acid sequence of YISSTSSTILYADTLKG (SEQ ID NO: 1173); a Kabat HCDR3 that comprises the amino acid sequence of RGAGAMDY (SEQ ID NO: 1181);
a Kabat LCDR1 that comprises the amino acid sequence of RASRSVNYIY (SEQ ID NO: 1155); a Kabat LCDR2 that comprises the amino acid sequence of YTSNLAP (SEQ ID NO: 1156); and a Kabat LCDR3 that comprises the amino acid sequence of QQFTSSPFV (SEQ ID NO: 1159).

Embodiment 25. A polypeptide molecule comprising an anti-TCRβV binding domain, wherein the binding domain comprises:
a Kabat HCDR1 that comprises the amino acid sequence of NTGMH (SEQ ID NO: 1170); a Kabat HCDR2 that comprises the amino acid sequence of YISSTSSTILYADTLKG (SEQ ID NO: 1173); a Kabat HCDR3 that comprises the amino acid sequence of RGEGAMDY (SEQ ID NO: 1180);
a Kabat LCDR1 that comprises the amino acid sequence of RASRSVNYAY (SEQ ID NO: 1151); a Kabat LCDR2 that comprises the amino acid sequence of YTSNLAP (SEQ ID NO: 1156); and a Kabat LCDR3 that comprises the amino acid sequence of QQFTSSPFV (SEQ ID NO: 1159).

Embodiment 26. A polypeptide molecule comprising an anti-TCRβV binding domain, wherein the binding domain comprises:
a Kabat HCDR1 that comprises the amino acid sequence of NTGMH (SEQ ID NO: 1170); a Kabat HCDR2 that comprises the amino acid sequence of YISSSSSTILYADTLKG (SEQ ID NO: 1174); a Kabat HCDR3 that comprises the amino acid sequence of RGAGAMDY (SEQ ID NO: 1181);
a Kabat LCDR1 that comprises the amino acid sequence of RASRSVNYAY (SEQ ID NO: 1151); a Kabat LCDR2 that comprises the amino acid sequence of YTSNLAP (SEQ ID NO: 1156); and a Kabat LCDR3 that comprises the amino acid sequence of QQFTSSPFV (SEQ ID NO: 1159).

Embodiment 27. A polypeptide molecule comprising an anti-TCRβV binding domain, wherein the binding domain comprises:
a Kabat HCDR1 that comprises the amino acid sequence of RTGMH (SEQ ID NO: 1163); a Kabat HCDR2 that comprises the amino acid sequence of YISSSSSTILYADTLKG (SEQ ID NO: 1174); a Kabat HCDR3 that comprises the amino acid sequence of RGEGAMDY (SEQ ID NO: 1180);
a Kabat LCDR1 that comprises the amino acid sequence of RASKSVNYAY (SEQ ID NO: 1152); a Kabat LCDR2 that comprises the amino acid sequence of YTSNLAP (SEQ ID NO: 1156); and a Kabat LCDR3 that comprises the amino acid sequence of QQFTSSPFV (SEQ ID NO: 1159).

Embodiment 28. A polypeptide molecule comprising an anti-TCRβV binding domain, wherein the binding domain comprises:
a Kabat HCDR1 that comprises the amino acid sequence of NTGMH (SEQ ID NO: 1170); a Kabat HCDR2 that comprises the amino acid sequence of YISSSSSTIYYADTLKG (SEQ ID NO: 1175); a Kabat HCDR3 that comprises the amino acid sequence of RGAGAMDY (SEQ ID NO: 1181);
a Kabat LCDR1 that comprises the amino acid sequence of RASRSVNYAY (SEQ ID NO: 1151); a Kabat LCDR2 that comprises the amino acid sequence of YTSNLAP (SEQ ID NO: 1156); and a Kabat LCDR3 that comprises the amino acid sequence of QQFTSSPFV (SEQ ID NO: 1159).

Embodiment 29. A polypeptide molecule comprising an anti-TCRβV binding domain, wherein the binding domain comprises:
a Kabat HCDR1 that comprises the amino acid sequence of ITGMH (SEQ ID NO: 1168); a Kabat HCDR2 that comprises the amino acid sequence of YISSSSSTILYADTLKG (SEQ ID NO: 1174); a Kabat HCDR3 that comprises the amino acid sequence of RGEGAMDY (SEQ ID NO: 1180);
a Kabat LCDR1 that comprises the amino acid sequence of RASRSVNYIY (SEQ ID NO: 1155); a Kabat LCDR2 that comprises the amino acid sequence of YTSNLAP (SEQ ID NO: 1156); and a Kabat LCDR3 that comprises the amino acid sequence of QQFTSSPFV (SEQ ID NO: 1159).

Embodiment 30. A polypeptide molecule comprising an anti-TCRβV-binding domain, wherein the binding domain comprises:
an IMGT HCDR1 that comprises an amino acid sequence selected from any one of: GFTFSIFG (SEQ ID NO: 1188), GFTFSRTG (SEQ ID NO: 1189), GFTFSIKG (SEQ ID NO: 1190), GFTFSNKG (SEQ ID NO: 1191), GFTFSRFG (SEQ ID NO: 1192), GFTFSRKG (SEQ ID NO: 1193), GFTFSITG (SEQ ID NO: 1194), GFTFSIYG (SEQ ID NO: 1195), and GFTFSNTG (SEQ ID NO: 1196);
an IMGT HCDR2 that comprises an amino acid sequence selected from any one of: ISSGSSTI (SEQ ID NO: 1197), ISSTSSTI (SEQ ID NO: 1198), ISSSSSTI (SEQ ID NO: 1199), and ISSWSSTI (SEQ ID NO: 1200);
an IMGT HCDR3 that comprises an amino acid sequence of ARRGEGAMDY (SEQ ID NO: 1201) or ARRGAGAMDY (SEQ ID NO: 1202);
an IMGT LCDR1 that comprises an amino acid sequence selected from any one of: SSVNY (SEQ ID NO: 1182), RSVNY (SEQ ID NO: 1184), and KSVNY (SEQ ID NO: 1185);
an IMGT LCDR2 that comprises an amino acid sequence of YTS;
an IMGT LCDR3 that comprises an amino acid sequence selected from any one of: QQFTSSPFT (SEQ ID NO: 1157) and QQFTSSPFV (SEQ ID NO: 1159);
wherein each of the HCDR1, HCDR2, HCDR3, LCDR1, LCDR2, and/or LCDR3 independently comprise 0, 1, 2, or 3 amino acid mutations.

Embodiment 31. The polypeptide molecule of embodiment 30, wherein the binding domain comprises:
(i) the IMGT HCDR1, the HCDR2, and the HCDR3 comprising the amino acid sequences of SEQ ID NOs: 1188, 1198, and 1201, respectively, and the IMGT LCDR1, the LCDR2, and the LCDR3 comprising the amino acid sequences of SEQ ID NOs: 1184, YTS, and 1159, respectively;
(ii) the IMGT HCDR1, the HCDR2, and the HCDR3 comprising the amino acid sequences of SEQ ID NOs: 1188, 1198, and 1201, respectively, and the IMGT LCDR1, the LCDR2, and the LCDR3 comprising the amino acid sequences of SEQ ID NOs: 1182, YTS, and 1159, respectively;
(iii) the IMGT HCDR1, the HCDR2, and the HCDR3 comprising the amino acid sequences of SEQ ID NOs: 1189, 1199, and 1201, respectively, and the IMGT LCDR1, the LCDR2, and the LCDR3 comprising the amino acid sequences of SEQ ID NOs: 1184, YTS, and 1159, respectively;
(iv) the IMGT HCDR1, the HCDR2, and the HCDR3 comprising the amino acid sequences of SEQ ID NOs: 1190, 1197, and 1202, respectively, and the IMGT LCDR1, the LCDR2, and the LCDR3 comprising the amino acid sequences of SEQ ID NOs: 1185, YTS, and 1159, respectively;
(v) the IMGT HCDR1, the HCDR2, and the HCDR3 comprising the amino acid sequences of SEQ ID NOs: 1191, 1199, and 1201, respectively, and the IMGT LCDR1, the LCDR2, and the LCDR3 comprising the amino acid sequences of SEQ ID NOs: 1185, YTS, and 1157, respectively;
(vi) the IMGT HCDR1, the HCDR2, and the HCDR3 comprising the amino acid sequences of SEQ ID NOs: 1186, 1200, and 1202, respectively, and the IMGT LCDR1, the LCDR2, and the LCDR3 comprising the amino acid sequences of SEQ ID NOs: 1182, YTS, and 1159, respectively;
(vii) the IMGT HCDR1, the HCDR2, and the HCDR3 comprising the amino acid sequences of SEQ ID NOs: 1188, 1200, and 1201, respectively, and the IMGT LCDR1, the LCDR2, and the LCDR3 comprising the amino acid sequences of SEQ ID NOs: 1184, YTS, and 1157, respectively;
(viii) the IMGT HCDR1, the HCDR2, and the HCDR3 comprising the amino acid sequences of SEQ ID NOs: 1192, 1200, and 1201, respectively, and the IMGT LCDR1, the LCDR2, and the LCDR3 comprising the amino acid sequences of SEQ ID NOs: 1185, YTS, and 1159, respectively;
(ix) the IMGT HCDR1, the HCDR2, and the HCDR3 comprising the amino acid sequences of SEQ ID NOs: 1193, 1198, and 1201, respectively, and the IMGT LCDR1, the LCDR2, and the LCDR3 comprising the amino acid sequences of SEQ ID NOs: 1184, YTS, and 1159, respectively;
(x) the IMGT HCDR1, the HCDR2, and the HCDR3 comprising the amino acid sequences of SEQ ID NOs: 1188, 1200, and 1202, respectively, and the IMGT LCDR1, the LCDR2, and the LCDR3 comprising the amino acid sequences of SEQ ID NOs: 1182, YTS, and 1159, respectively;
(xi) the IMGT HCDR1, the HCDR2, and the HCDR3 comprising the amino acid sequences of SEQ ID NOs: 1188, 1199, and 1202, respectively, and the IMGT LCDR1, the LCDR2, and the LCDR3 comprising the amino acid sequences of SEQ ID NOs: 1182, YTS, and 1159, respectively;
(xii) the IMGT HCDR1, the HCDR2, and the HCDR3 comprising the amino acid sequences of SEQ ID NOs: 1191, 1199, and 1202, respectively, and the IMGT LCDR1, the LCDR2, and the LCDR3 comprising the amino acid sequences of SEQ ID NOs: 1182, YTS, and 1157, respectively;
(xiii) the IMGT HCDR1, the HCDR2, and the HCDR3 comprising the amino acid sequences of SEQ ID NOs: 1192, 1198, and 1201, respectively, and the IMGT LCDR1, the LCDR2, and the LCDR3 comprising the amino acid sequences of SEQ ID NOs: 1184, YTS, and 1157, respectively;
(xiv) the IMGT HCDR1, the HCDR2, and the HCDR3 comprising the amino acid sequences of SEQ ID NOs: 1194, 1198, and 1202, respectively, and the IMGT LCDR1, the LCDR2, and the LCDR3 comprising the amino acid sequences of SEQ ID NOs: 1185, YTS, and 1159, respectively;
(xv) the IMGT HCDR1, the HCDR2, and the HCDR3 comprising the amino acid sequences of SEQ ID NOs: 1193, 1197, and 1202, respectively, and the IMGT LCDR1, the LCDR2, and the LCDR3 comprising the amino acid sequences of SEQ ID NOs: 1182, YTS, and 1157, respectively;
(xvi) the IMGT HCDR1, the HCDR2, and the HCDR3 comprising the amino acid sequences of SEQ ID NOs: 1194, 1200, and 1202, respectively, and the IMGT LCDR1, the LCDR2, and the LCDR3 comprising the amino acid sequences of SEQ ID NOs: 1185, YTS, and 1157, respectively;
(xvii) the IMGT HCDR1, the HCDR2, and the HCDR3 comprising the amino acid sequences of SEQ ID NOs: 1190, 1199, and 1202, respectively, and the IMGT LCDR1, the LCDR2, and the LCDR3 comprising the amino acid sequences of SEQ ID NOs: 1185, YTS, and 1159, respectively;
(xviii) the IMGT HCDR1, the HCDR2, and the HCDR3 comprising the amino acid sequences of SEQ ID NOs: 1193, 1200, and 1202, respectively, and the IMGT LCDR1, the LCDR2, and the LCDR3 comprising the amino acid sequences of SEQ ID NOs: 1182, YTS, and 1157, respectively;
(xix) the IMGT HCDR1, the HCDR2, and the HCDR3 comprising the amino acid sequences of SEQ ID NOs: 1188, 1198, and 1201, respectively, and the IMGT LCDR1, the LCDR2, and the LCDR3 comprising the amino acid sequences of SEQ ID NOs: 1184, YTS, and 1157, respectively;
(xx) the IMGT HCDR1, the HCDR2, and the HCDR3 comprising the amino acid sequences of SEQ ID NOs: 1195, 1198, and 1202, respectively, and the IMGT LCDR1, the LCDR2, and the LCDR3 comprising the amino acid sequences of SEQ ID NOs: 1182, YTS, and 1157, respectively;
(xxi) the IMGT HCDR1, the HCDR2, and the HCDR3 comprising the amino acid sequences of SEQ ID NOs: 1189, 1197, and 1201, respectively, and the IMGT LCDR1, the LCDR2, and the LCDR3 comprising the amino acid sequences of SEQ ID NOs: 1185, YTS, and 1159, respectively;
(xxii) the IMGT HCDR1, the HCDR2, and the HCDR3 comprising the amino acid sequences of SEQ ID NOs: 1196, 1198, and 1202, respectively, and the IMGT LCDR1, the LCDR2, and the LCDR3 comprising the amino acid sequences of SEQ ID NOs: 1184, YTS, and 1157, respectively;
(xxiii) the IMGT HCDR1, the HCDR2, and the HCDR3 comprising the amino acid sequences of SEQ ID NOs: 1191, 1197, and 1201, respectively, and the IMGT LCDR1, the LCDR2, and the LCDR3 comprising the amino acid sequences of SEQ ID NOs: 1185, YTS, and 1157, respectively;
(xxiv) the IMGT HCDR1, the HCDR2, and the HCDR3 comprising the amino acid sequences of SEQ ID NOs: 1194, 1198, and 1202, respectively, and the IMGT LCDR1, the LCDR2, and the LCDR3 comprising the amino acid sequences of SEQ ID NOs: 1184, YTS, and 1159, respectively;
(xxv) the IMGT HCDR1, the HCDR2, and the HCDR3 comprising the amino acid sequences of SEQ ID NOs: 1192, 1198, and 1201, respectively, and the IMGT LCDR1, the LCDR2, and the LCDR3 comprising the amino acid sequences of SEQ ID NOs: 1184, YTS, and 1159, respectively;
(xxvi) the IMGT HCDR1, the HCDR2, and the HCDR3 comprising the amino acid sequences of SEQ ID NOs: 1194, 1199, and 1202, respectively, and the IMGT LCDR1, the LCDR2, and the LCDR3 comprising the amino acid sequences of SEQ ID NOs: 1185, YTS, and 1159, respectively;
(xxvii) the IMGT HCDR1, the HCDR2, and the HCDR3 comprising the amino acid sequences of SEQ ID NOs: 1189, 1200, and 1201, respectively, and the IMGT LCDR1, the LCDR2, and the LCDR3 comprising the amino acid sequences of SEQ ID NOs: 1182, YTS, and 1159, respectively;
(xxviii) the IMGT HCDR1, the HCDR2, and the HCDR3 comprising the amino acid sequences of SEQ ID NOs: 1193, 1200, and 1202, respectively, and the IMGT LCDR1, the LCDR2, and the LCDR3 comprising the amino acid sequences of SEQ ID NOs: 1185, YTS, and 1159, respectively;
(xxix) the IMGT HCDR1, the HCDR2, and the HCDR3 comprising the amino acid sequences of SEQ ID NOs: 1186, 1200, and 1202, respectively, and the IMGT LCDR1, the LCDR2, and the LCDR3 comprising the amino acid sequences of SEQ ID NOs: 1185, YTS, and 1159, respectively;
(xxx) the IMGT HCDR1, the HCDR2, and the HCDR3 comprising the amino acid sequences of SEQ ID NOs: 1196, 1198, and 1201, respectively, and the IMGT LCDR1, the LCDR2, and the LCDR3 comprising the amino acid sequences of SEQ ID NOs: 1184, YTS, and 1159, respectively;
(xxxi) the IMGT HCDR1, the HCDR2, and the HCDR3 comprising the amino acid sequences of SEQ ID NOs: 1196, 1199, and 1202, respectively, and the IMGT LCDR1, the LCDR2, and the LCDR3 comprising the amino acid sequences of SEQ ID NOs: 1184, YTS, and 1159, respectively;
(xxxii) the IMGT HCDR1, the HCDR2, and the HCDR3 comprising the amino acid sequences of SEQ ID NOs: 1189, 1200, and 1202, respectively, and the IMGT LCDR1, the LCDR2, and the LCDR3 comprising the amino acid sequences of SEQ ID NOs: 1184, YTS, and 1159, respectively;
(xxxiii) the IMGT HCDR1, the HCDR2, and the HCDR3 comprising the amino acid sequences of SEQ ID NOs: 1191, 1200, and 1202, respectively, and the IMGT LCDR1, the LCDR2, and the LCDR3 comprising the amino acid sequences of SEQ ID NOs: 1182, YTS, and 1157, respectively;
(xxxiv) the IMGT HCDR1, the HCDR2, and the HCDR3 comprising the amino acid sequences of SEQ ID NOs: 1191, 1197, and 1202, respectively, and the IMGT LCDR1, the LCDR2, and the LCDR3 comprising the amino acid sequences of SEQ ID NOs: 1184, YTS, and 1159, respectively;
(xxxv) the IMGT HCDR1, the HCDR2, and the HCDR3 comprising the amino acid sequences of SEQ ID NOs: 1189, 1199, and 1201, respectively, and the IMGT LCDR1, the LCDR2, and the LCDR3 comprising the amino acid sequences of SEQ ID NOs: 1185, YTS, and 1159, respectively;
(xxxvi) the IMGT HCDR1, the HCDR2, and the HCDR3 comprising the amino acid sequences of SEQ ID NOs: 1196, 1199, and 1202, respectively, and the IMGT LCDR1, the LCDR2, and the LCDR3 comprising the amino acid sequences of SEQ ID NOs: 1184, YTS, and 1159, respectively; or
(xxxvii) the IMGT HCDR1, the HCDR2, and the HCDR3 comprising the amino acid sequences of SEQ ID NOs: 1194, 1199, and 1201, respectively, and the IMGT LCDR1, the LCDR2, and the LCDR3 comprising the amino acid sequences of SEQ ID NOs: 1184, YTS, and 1159, respectively.

Embodiment 32. A polypeptide molecule comprising an anti-TCRβV binding domain, wherein the binding domain comprises:
an IMGT HCDR1 that comprises the amino acid sequence of GFTFSNTG (SEQ ID NO: 1196); an IMGT HCDR2 that comprises the amino acid sequence of ISSTSSTI (SEQ ID NO: 1198);
an IMGT HCDR3 that comprises the amino acid sequence of ARRGAGAMDY (SEQ ID NO: 1202);
an IMGT LCDR1 that comprises the amino acid sequence of RSVNY (SEQ ID NO: 1184);
an IMGT LCDR2 that comprises the amino acid sequence of YTS; and an IMGT LCDR3 that comprises the amino acid sequence of QQFTSSPFT (SEQ ID NO: 1157).

Embodiment 33. A polypeptide molecule comprising an anti-TCRβV binding domain, wherein the binding domain comprises:
an IMGT HCDR1 that comprises the amino acid sequence of GFTFSITG (SEQ ID NO: 1194); an IMGT HCDR2 that comprises the amino acid sequence of ISSTSSTI (SEQ ID NO: 1198); an IMGT HCDR3 that comprises the amino acid sequence of ARRGAGAMDY (SEQ ID NO: 1202);
an IMGT LCDR1 that comprises the amino acid sequence of RSVNY (SEQ ID NO: 1184); an IMGT LCDR2 that comprises the amino acid sequence of YTS; and an IMGT LCDR3 that comprises the amino acid sequence of QQFTSSPFV (SEQ ID NO: 1159).

Embodiment 34. A polypeptide molecule comprising an anti-TCRβV binding domain, wherein the binding domain comprises:
an IMGT HCDR1 that comprises the amino acid sequence of GFTFSNTG (SEQ ID NO: 1196); an IMGT HCDR2 that comprises the amino acid sequence of ISSTSSTI (SEQ ID NO: 1198); an IMGT HCDR3 that comprises the amino acid sequence of ARRGEGAMDY (SEQ ID NO: 1201);
an IMGT LCDR1 that comprises the amino acid sequence of RSVNY (SEQ ID NO: 1184); an IMGT LCDR2 that comprises the amino acid sequence of YTS; and an IMGT LCDR3 that comprises the amino acid sequence of QQFTSSPFV (SEQ ID NO: 1159).

Embodiment 35. A polypeptide molecule comprising an anti-TCRβV binding domain, wherein the binding domain comprises:
an IMGT HCDR1 that comprises the amino acid sequence of GFTFSNTG (SEQ ID NO: 1196); an IMGT HCDR2 that comprises the amino acid sequence of ISSSSSTI (SEQ ID NO: 1199); an IMGT HCDR3 that comprises the amino acid sequence of ARRGAGAMDY (SEQ ID NO: 1202);
an IMGT LCDR1 that comprises the amino acid sequence of RSVNY (SEQ ID NO: 1184); an IMGT LCDR2 that comprises the amino acid sequence of YTS; and an IMGT LCDR3 that comprises the amino acid sequence of QQFTSSPFV (SEQ ID NO: 1159).

Embodiment 36. A polypeptide molecule comprising an anti-TCRβV binding domain, wherein the binding domain comprises:
an IMGT HCDR1 that comprises the amino acid sequence of GFTFSRTG (SEQ ID NO: 1189); an IMGT HCDR2 that comprises the amino acid sequence of ISSSSSTI (SEQ ID NO: 1199); an IMGT HCDR3 that comprises the amino acid sequence of ARRGEGAMDY (SEQ ID NO: 1201);
an IMGT LCDR1 that comprises the amino acid sequence of KSVNY (SEQ ID NO: 1185); an IMGT LCDR2 that comprises the amino acid sequence of YTS; and an IMGT LCDR3 that comprises the amino acid sequence of QQFTSSPFV (SEQ ID NO: 1159).

Embodiment 37. A polypeptide molecule comprising an anti-TCRβV binding domain, wherein the binding domain comprises:
an IMGT HCDR1 that comprises the amino acid sequence of GFTFSNTG (SEQ ID NO: 1196); an IMGT HCDR2 that comprises the amino acid sequence of ISSSSSTI (SEQ ID NO: 1199);
an IMGT HCDR3 that comprises the amino acid sequence of ARRGAGAMDY (SEQ ID NO: 1202);
an IMGT LCDR1 that comprises the amino acid sequence of RSVNY (SEQ ID NO: 1184);
an IMGT LCDR2 that comprises the amino acid sequence of YTS; and an IMGT LCDR3 that comprises the amino acid sequence of QQFTSSPFV (SEQ ID NO: 1159).

Embodiment 38. A polypeptide molecule comprising an anti-TCRβV binding domain, wherein the binding domain comprises:
an IMGT HCDR1 that comprises the amino acid sequence of GFTFSITG (SEQ ID NO: 1194); an IMGT HCDR2 that comprises the amino acid sequence of ISSSSSTI (SEQ ID NO: 1199); an IMGT HCDR3 that comprises the amino acid sequence of ARRGEGAMDY (SEQ ID NO: 1201);
an IMGT LCDR1 that comprises the amino acid sequence of RSVNY (SEQ ID NO: 1184); an IMGT LCDR2 that comprises the amino acid sequence of YTS; and an IMGT LCDR3 that comprises the amino acid sequence of QQFTSSPFV (SEQ ID NO: 1159).

Embodiment 39. A polypeptide molecule comprising an anti-T cell receptor beta variable chain (TCRβV) binding domain comprising:
(i) a variable heavy chain (VH) region comprising an HCDR1, an HCDR2, and an HCDR3, wherein the VH region comprises an amino acid sequence of any one of SEQ ID NOs: 100-111, 2512, and 2513; and
(ii) a variable light chain (VL) region comprising an LCDR1, an LCDR2, and an LCDR3, wherein the VL region comprises an amino acid sequence of any one of SEQ ID NOs: 85-93 and 2514;
wherein the HCDR1-3 and LCDR1-3 sequences are each determined according to the Kabat numbering scheme, the Chothia numbering scheme, or the ImMunoGeneTics Information System (IMGT).

Embodiment 40. The polypeptide molecule of any one of embodiments 1-38, wherein the polypeptide molecule comprises a VH region and/or a VL region.

Embodiment 41. The polypeptide molecule of embodiment 40, wherein the VH region comprises an amino acid sequence that is at least about 80%, at least about 85%, at least about 90%, at least about 95%, at least about 96%, at least about 97%, at least about 98%, at least about 99%, or 100% identical to any one of SEQ ID NOs: 100-111, 2512, and 2513.

Embodiment 42. The polypeptide molecule of embodiment 40 or 41, wherein the VL region comprises an amino acid sequence that is at least about 80%, at least about 85%, at least about 90%, at least about 95%, at least about 96%, at least about 97%, at least about 98%, at least about 99%, or 100% identical to any one of SEQ ID NOs: 85-93 and 2514.

Embodiment 43. The polypeptide molecule of any one of embodiments 40-42, wherein the VH region comprises an amino acid sequence that is at least about 80%, at least about 85%, at least about 90%, at least about 95%, at least about 96%, at least about 97%, at least about 98%, at least about 99%, or 100% identical to any one of SEQ ID NOs: 100-111, 2512, and 2513; and the VL region comprises an amino acid sequence that is at least about 80%, at least about 85%, at least about 90%, at least about 95%, at least about 96%, at least about 97%, at least about 98%, at least about 99%, or 100% identical to any one of SEQ ID NOs: 85-93 and 2514.

Embodiment 44. The polypeptide molecule of embodiment 43, wherein the VH region has a C44 mutation and the VL region has a C100 mutation.

Embodiment 45. The polypeptide molecule of any one of embodiments 40-44, wherein the VH region comprises an amino acid sequence that is at least about 80%, at least about 85%, at least about 90%, at least about 95%, at least about 96%, at least about 97%, at least about 98%, at least about 99%, or 100% identical to SEQ ID NO: 100; and the VL region comprises an amino acid sequence that is at least about 80%, at least about 85%, at least about 90%, at least about 95%, at least about 96%, at least about 97%, at least about 98%, at least about 99%, or 100% identical to SEQ ID NO: 85.

Embodiment 46. The polypeptide molecule of any one of embodiments 40-44, wherein the VH region comprises an amino acid sequence that is at least about 80%, at least about 85%, at least about 90%, at least about 95%, at least about 96%, at least about 97%, at least about 98%, at least about 99%, or 100% identical to SEQ ID NO: 101; and the VL region comprises an amino acid sequence that is at least about 80%, at least about 85%, at least about 90%, at least about 95%, at least about 96%, at least about 97%, at least about 98%, at least about 99%, or 100% identical to SEQ ID NO: 86.

Embodiment 47. The polypeptide molecule of any one of embodiments 40-44, wherein the VH region comprises an amino acid sequence that is at least about 80%, at least about 85%, at least about 90%, at least about 95%, at least about 96%, at least about 97%, at least about 98%, at least about 99%, or 100% identical to SEQ ID NO: 102; and the VL region comprises an amino acid sequence that is at least about 80%, at least about 85%, at least about 90%, at least about 95%, at least about 96%, at least about 97%, at least about 98%, at least about 99%, or 100% identical to SEQ ID NO: 87.

Embodiment 48. The polypeptide molecule of any one of embodiments 40-44, wherein the VH region comprises an amino acid sequence that is at least about 80%, at least about 85%, at least about 90%, at least about 95%, at least about 96%, at least about 97%, at least about 98%, at least about 99%, or 100% identical to SEQ ID NO: 85; and the VL region comprises an amino acid sequence that is at least about 80%, at least about 85%, at least about 90%, at least about 95%, at least about 96%, at least about 97%, at least about 98%, at least about 99%, or 100% identical to SEQ ID NO: 101.

Embodiment 49. The polypeptide molecule of any one of embodiments 40-44, wherein the VH region comprises an amino acid sequence that is at least about 80%, at least about 85%, at least about 90%, at least about 95%, at least about 96%, at least about 97%, at least about 98%, at least about 99%, or 100% identical to SEQ ID NO: 103; and the VL region comprises an amino acid sequence that is at least about 80%, at least about 85%, at least about 90%, at least about 95%, at least about 96%, at least about 97%, at least about 98%, at least about 99%, or 100% identical to SEQ ID NO: 85.

Embodiment 50. The polypeptide molecule of any one of embodiments 40-44, wherein the VH region comprises an amino acid sequence that is at least about 80%, at least about 85%, at least about 90%, at least about 95%, at least about 96%, at least about 97%, at least about 98%, at least about 99%, or 100% identical to SEQ ID NO: 104; and the VL region comprises an amino acid sequence that is at least about 80%, at least about 85%, at least about 90%, at least about 95%, at least about 96%, at least about 97%, at least about 98%, at least about 99%, or 100% identical to SEQ ID NO: 88.

Embodiment 51. The polypeptide molecule of any one of embodiments 40-44, wherein the VH region comprises an amino acid sequence that is at least about 80%, at least about 85%, at least about 90%, at least about 95%, at least about 96%, at least about 97%, at least about 98%, at least about 99%, or 100% identical to SEQ ID NO: 105; and the VL region comprises an amino acid sequence that is at least about 80%, at least about 85%, at least about 90%, at least about 95%, at least about 96%, at least about 97%, at least about 98%, at least about 99%, or 100% identical to SEQ ID NO: 89.

Embodiment 52. The polypeptide molecule of any one of embodiments 40-44, wherein the VH region comprises an amino acid sequence that is at least about 80%, at least about 85%, at least about 90%, at least about 95%, at least about 96%, at least about 97%, at least about 98%, at least about 99%, or 100% identical to SEQ ID NO: 101; and the VL region comprises an amino acid sequence that is at least about 80%, at least about 85%, at least about 90%, at least about 95%, at least about 96%, at least about 97%, at least about 98%, at least about 99%, or 100% identical to SEQ ID NO: 91.

Embodiment 53. A polypeptide molecule comprising an anti-TCRβV binding domain comprising:
(i) a VH region comprising an HCDR1, an HCDR2, and an HCDR3, wherein the VH region comprises an amino acid sequence of any one of SEQ ID NOs: 1116-1147; and
(ii) a VL region comprising an LCDR1, an LCDR2, and an LCDR3, wherein the VL region comprises an amino acid sequence of any one of SEQ ID NOs: 1104-1112;
wherein the HCDR1-3 and LCDR1-3 sequences are each determined according to the Kabat numbering scheme, the Chothia numbering scheme, or the ImMunoGeneTics Information System (IMGT).

Embodiment 54. The polypeptide molecule of embodiment 40, wherein the VH region comprises an amino acid sequence that is at least about 80%, at least about 85%, at least about 90%, at least about 95%, at least about 96%, at least about 97%, at least about 98%, at least about 99%, or 100% identical to any one of SEQ ID NOs: 1116-1147.

Embodiment 55. The polypeptide molecule of embodiment 40 or 54, wherein the VL region comprises an amino acid sequence that is at least about 80%, at least about 85%, at least about 90%, at least about 95%, at least about 96%, at least about 97%, at least about 98%, at least about 99%, or 100% identical to any one of SEQ ID NOs: 1104-1112.

Embodiment 56. The polypeptide molecule of embodiment 54 or 55, wherein the VH region comprises an amino acid sequence that is at least about 80%, at least about 85%, at least about 90%, at least about 95%, at least about 96%, at least about 97%, at least about 98%, at least about 99%, or 100% identical to any one of SEQ ID NOs: 1116-1147; and the VL region comprises an amino acid sequence that is at least about 80%, at least about 85%, at least about 90%, at least about 95%, at least about 96%, at least about 97%, at least about 98%, at least about 99%, or 100% identical to any one of SEQ ID NOs: 1104-1112.

Embodiment 57. The polypeptide molecule of embodiment 56, wherein the VH region has a C44 mutation and the VL region has a C100 mutation.

Embodiment 58. The polypeptide molecule of any one of embodiments 54-57, wherein the VH region comprises an amino acid sequence that is at least about 80%, at least about 85%, at least about 90%, at least about 95%, at least about 96%, at least about 97%, at least about 98%, at least about 99%, or 100% identical to SEQ ID NO: 1135; and the VL region comprises an amino acid sequence that is at least about 80%, at least about 85%, at least about 90%, at least about 95%, at least about 96%, at least about 97%, at least about 98%, at least about 99%, or 100% identical to SEQ ID NO: 1108.

Embodiment 59. The polypeptide molecule of any one of embodiments 54-57, wherein the VH region comprises an amino acid sequence that is at least about 80%, at least about 85%, at least about 90%, at least about 95%, at least about 96%, at least about 97%, at least about 98%, at least about 99%, or 100% identical to SEQ ID NO: 1137; and the VL region comprises an amino acid sequence that is at least about 80%, at least about 85%, at least about 90%, at least about 95%, at least about 96%, at least about 97%, at least about 98%, at least about 99%, or 100% identical to SEQ ID NO: 1110.

Embodiment 60. The polypeptide molecule of any one of embodiments 54-57, wherein the VH region comprises an amino acid sequence that is at least about 80%, at least about 85%, at least about 90%, at least about 95%, at least about 96%, at least about 97%, at least about 98%, at least about 99%, or 100% identical to SEQ ID NO: 1141; and the VL region comprises an amino acid sequence that is at least about 80%, at least about 85%, at least about 90%, at least about 95%, at least about 96%, at least about 97%, at least about 98%, at least about 99%, or 100% identical to SEQ ID NO: 1104.

Embodiment 61. The polypeptide molecule of any one of embodiments 54-57, wherein the VH region comprises an amino acid sequence that is at least about 80%, at least about 85%, at least about 90%, at least about 95%, at least about 96%, at least about 97%, at least about 98%, at least about 99%, or 100% identical to SEQ ID NO: 1142; and the VL region comprises an amino acid sequence that is at least about 80%, at least about 85%, at least about 90%, at least about 95%, at least about 96%, at least about 97%, at least about 98%, at least about 99%, or 100% identical to SEQ ID NO: 1104.

Embodiment 62. The polypeptide molecule of any one of embodiments 54-57, wherein the VH region comprises an amino acid sequence that is at least about 80%, at least about 85%, at least about 90%, at least about 95%, at least about 96%, at least about 97%, at least about 98%, at least about 99%, or 100% identical to SEQ ID NO: 1117; and the VL region comprises an amino acid sequence that is at least about 80%, at least about 85%, at least about 90%, at least about 95%, at least about 96%, at least about 97%, at least about 98%, at least about 99%, or 100% identical to SEQ ID NO: 1106.

Embodiment 63. The polypeptide molecule of any one of embodiments 54-57, wherein the VH region comprises an amino acid sequence that is at least about 80%, at least about 85%, at least about 90%, at least about 95%, at least about 96%, at least about 97%, at least about 98%, at least about 99%, or 100% identical to SEQ ID NO: 1146; and the VL region comprises an amino acid sequence that is at least about 80%, at least about 85%, at least about 90%, at least about 95%, at least about 96%, at least about 97%, at least about 98%, at least about 99%, or 100% identical to SEQ ID NO: 1104.

Embodiment 64. The polypeptide molecule of any one of embodiments 54-57, wherein the VH region comprises an amino acid sequence that is at least about 80%, at least about 85%, at least about 90%, at least about 95%, at least about 96%, at least about 97%, at least about 98%, at least about 99%, or 100% identical to SEQ ID NO: 1147; and the VL region comprises an amino acid sequence that is at least about 80%, at least about 85%, at least about 90%, at least about 95%, at least about 96%, at least about 97%, at least about 98%, at least about 99%, or 100% identical to SEQ ID NO: 1110.

Embodiment 65. The polypeptide molecule of any one of embodiments 1-64, wherein the TCRβV-binding domain is a single chain variable fragment (scFv), Fab, CrossFab, Fab′, or F(ab′)₂.

Embodiment 66. The polypeptide molecule of any one of embodiments 1-65, wherein the TCRβV-binding domain is an scFv comprising a VH region and a VL region.

Embodiment 67. The polypeptide molecule of embodiment 66, wherein the C-terminus of the VH region is linked to the N-terminus of the VL region.

Embodiment 68. The polypeptide molecule of embodiment 66, wherein the C-terminus of the VL region is linked to the N-terminus of the VH region.

Embodiment 69. The polypeptide of any one of embodiments 66-68, wherein the VH region and VL region are linked by a peptide linker.

Embodiment 70. The polypeptide molecule of embodiment 69, wherein the peptide linker comprises the formula of (Gly4Ser)ₙ, wherein n=1-5 (SEQ ID NO: 1424).

Embodiment 71. The polypeptide molecule of embodiment 69, wherein the peptide linker is (Gly₄Ser)₃ (SEQ ID NO: 1342).

Embodiment 72. The polypeptide molecule of embodiment 69, wherein the peptide linker is (Gly₄Ser) (SEQ ID NO: 1425).

Embodiment 73. The polypeptide of any one of embodiments 66-72, wherein the VH region and the VL region are linked by one or more engineered disulfide bond(s).

Embodiment 74. The binding molecule of embodiment 73, wherein one cysteine that forms the engineered disulfide bond is located in the VH region, and wherein the other cysteine that forms the engineered disulfide bond is located in the VL region.

Embodiment 75. The binding molecule of embodiment 73 or 74, wherein the positions of the one or more engineered disulfide bond(s) are selected from the group consisting of: VH44-VL100, VH100B-VL49, VH100-VL50, VH101-VL46, and VH105-VL43, according to EU numbering.

Embodiment 76. The polypeptide molecule of embodiment 66, wherein the scFv comprises an amino acid sequence that is at least about 80%, at least about 85%, at least about 90%, at least about 95%, at least about 96%, at least about 97%, at least about 98%, at least about 99%, or 100% identical to any one of SEQ ID NOs: 149-222.

Embodiment 77. The polypeptide molecule of embodiment 66, wherein the scFv comprises an amino acid sequence that is at least about 80%, at least about 85%, at least about 90%, at least about 95%, at least about 96%, at least about 97%, at least about 98%, at least about 99%, or 100% identical to SEQ ID NO: 205.

Embodiment 78. The polypeptide molecule of embodiment 66, wherein the scFv comprises an amino acid sequence that is at least about 80%, at least about 85%, at least about 90%, at least about 95%, at least about 96%, at least about 97%, at least about 98%, at least about 99%, or 100% identical to SEQ ID NO: 206.

Embodiment 79. The polypeptide molecule of embodiment 66, wherein the scFv comprises an amino acid sequence that is at least about 80%, at least about 85%, at least about 90%, at least about 95%, at least about 96%, at least about 97%, at least about 98%, at least about 99%, or 100% identical to SEQ ID NO: 207.

Embodiment 80. The polypeptide molecule of embodiment 66, wherein the scFv comprises an amino acid sequence that is at least about 80%, at least about 85%, at least about 90%, at least about 95%, at least about 96%, at least about 97%, at least about 98%, at least about 99%, or 100% identical to SEQ ID NO: 208.

Embodiment 81. The polypeptide molecule of embodiment 66, wherein the scFv comprises an amino acid sequence that is at least about 80%, at least about 85%, at least about 90%, at least about 95%, at least about 96%, at least about 97%, at least about 98%, at least about 99%, or 100% identical to SEQ ID NO: 209.

Embodiment 82. The polypeptide molecule of embodiment 66, wherein the scFv comprises an amino acid sequence that is at least about 80%, at least about 85%, at least about 90%, at least about 95%, at least about 96%, at least about 97%, at least about 98%, at least about 99%, or 100% identical to SEQ ID NO: 210.

Embodiment 83. The polypeptide molecule of embodiment 66, wherein the scFv comprises an amino acid sequence that is at least about 80%, at least about 85%, at least about 90%, at least about 95%, at least about 96%, at least about 97%, at least about 98%, at least about 99%, or 100% identical to SEQ ID NO: 211.

Embodiment 84. The polypeptide molecule of embodiment 66, wherein the scFv comprises an amino acid sequence that is at least about 80%, at least about 85%, at least about 90%, at least about 95%, at least about 96%, at least about 97%, at least about 98%, at least about 99%, or 100% identical to SEQ ID NO: 212.

Embodiment 85. The polypeptide molecule of embodiment 66, wherein the scFv comprises an amino acid sequence that is at least about 80%, at least about 85%, at least about 90%, at least about 95%, at least about 96%, at least about 97%, at least about 98%, at least about 99%, or 100% identical to any one of SEQ ID NOs: 1203-1277.

Embodiment 86. The polypeptide molecule of embodiment 66, wherein the scFv comprises an amino acid sequence that is at least about 80%, at least about 85%, at least about 90%, at least about 95%, at least about 96%, at least about 97%, at least about 98%, at least about 99%, or 100% identical to SEQ ID NO: 1262.

Embodiment 87. The polypeptide molecule of embodiment 66, wherein the scFv comprises an amino acid sequence that is at least about 80%, at least about 85%, at least about 90%, at least about 95%, at least about 96%, at least about 97%, at least about 98%, at least about 99%, or 100% identical to SEQ ID NO: 1264.

Embodiment 88. The polypeptide molecule of embodiment 66, wherein the scFv comprises an amino acid sequence that is at least about 80%, at least about 85%, at least about 90%, at least about 95%, at least about 96%, at least about 97%, at least about 98%, at least about 99%, or 100% identical to SEQ ID NO: 1270.

Embodiment 89. The polypeptide molecule of embodiment 66, wherein the scFv comprises an amino acid sequence that is at least about 80%, at least about 85%, at least about 90%, at least about 95%, at least about 96%, at least about 97%, at least about 98%, at least about 99%, or 100% identical to SEQ ID NO: 1271.

Embodiment 90. The polypeptide molecule of embodiment 66, wherein the scFv comprises an amino acid sequence that is at least about 80%, at least about 85%, at least about 90%, at least about 95%, at least about 96%, at least about 97%, at least about 98%, at least about 99%, or 100% identical to SEQ ID NO: 1275.

Embodiment 91. The polypeptide molecule of embodiment 66, wherein the scFv comprises an amino acid sequence that is at least about 80%, at least about 85%, at least about 90%, at least about 95%, at least about 96%, at least about 97%, at least about 98%, at least about 99%, or 100% identical to SEQ ID NO: 1276.

Embodiment 92. The polypeptide molecule of embodiment 66, wherein the scFv comprises an amino acid sequence that is at least about 80%, at least about 85%, at least about 90%, at least about 95%, at least about 96%, at least about 97%, at least about 98%, at least about 99%, or 100% identical to SEQ ID NO: 1277.

Embodiment 93. The polypeptide molecule of any one of embodiments 1-65, wherein the TCRβV-binding domain is a Fab comprising the VH region, the VL region, a first heavy chain constant region (CH1), and a light chain constant region (CL), wherein the C-terminus of the VH region is connected to the N-terminus of the CH1 region, and the C-terminus of the VL region is connected to the N-terminus of the CL region.

Embodiment 94. The polypeptide molecule of any one of embodiments 1-65, wherein the TCRβV-binding domain is a CrossFab comprising the VH region, the VL region, a first heavy chain constant region (CH1), and a light chain constant region (CL), wherein the C-terminus of the VH region is connected to the N-terminus of the CL region, and the C-terminus of the VL region is connected to the N-terminus of the CH1 region.

Embodiment 95. The polypeptide molecule of embodiment 93 or 94, wherein the CH1 region and the CL region are connected by a disulfide bond.

Embodiment 96. The polypeptide molecule of any one of embodiments 93-95, wherein TCRβV-binding domain is a charge steering Fab or charge steering CrossFab.

Embodiment 97. The polypeptide molecule of embodiment 96, wherein the CH1 region of the charge steering Fab or charge steering CrossFab comprises mutations K147E and/or K213E according to EU numbering, and the CL region of the charge steering Fab or charge steering CrossFab comprises mutations E123R and/or Q124K according to EU numbering.

Embodiment 98. The polypeptide molecule of any one of embodiments 93-97, wherein the Fab or CrossFab comprises (i) a light chain sequence that is at least about 80%, at least about 85%, at least about 90%, at least about 95%, at least about 96%, at least about 97%, at least about 98%, at least about 99%, or 100% identical to any one of SEQ ID NOs: 230-574; and (ii) a heavy chain sequence that is at least about 80%, at least about 85%, at least about 90%, at least about 95%, at least about 96%, at least about 97%, at least about 98%, at least about 99%, or 100% identical to any one of SEQ ID NOs: 575-1084.

Embodiment 99. The polypeptide molecule of embodiment 98, wherein the Fab or CrossFab comprises (i) a light chain sequence that is at least about 80%, at least about 85%, at least about 90%, at least about 95%, at least about 96%, at least about 97%, at least about 98%, at least about 99%, or 100% identical to SEQ ID NO: 236; and (ii) a heavy chain sequence that is at least about 80%, at least about 85%, at least about 90%, at least about 95%, at least about 96%, at least about 97%, at least about 98%, at least about 99%, or 100% identical to SEQ ID NO: 582.

Embodiment 100. The polypeptide molecule of embodiment 98, wherein the Fab or CrossFab comprises (i) a light chain sequence that is at least about 80%, at least about 85%, at least about 90%, at least about 95%, at least about 96%, at least about 97%, at least about 98%, at least about 99%, or 100% identical to SEQ ID NO: 237; and (ii) a heavy chain sequence that is at least about 80%, at least about 85%, at least about 90%, at least about 95%, at least about 96%, at least about 97%, at least about 98%, at least about 99%, or 100% identical to SEQ ID NO: 583.

Embodiment 101. The polypeptide molecule of embodiment 98, wherein the Fab or CrossFab comprises (i) a light chain sequence that is at least about 80%, at least about 85%, at least about 90%, at least about 95%, at least about 96%, at least about 97%, at least about 98%, at least about 99%, or 100% identical to SEQ ID NO: 238; and (ii) a heavy chain sequence that is at least about 80%, at least about 85%, at least about 90%, at least about 95%, at least about 96%, at least about 97%, at least about 98%, at least about 99%, or 100% identical to SEQ ID NO: 584.

Embodiment 102. The polypeptide molecule of embodiment 98, wherein the Fab or CrossFab comprises (i) a light chain sequence that is at least about 80%, at least about 85%, at least about 90%, at least about 95%, at least about 96%, at least about 97%, at least about 98%, at least about 99%, or 100% identical to SEQ ID NO: 236; and (ii) a heavy chain sequence that is at least about 80%, at least about 85%, at least about 90%, at least about 95%, at least about 96%, at least about 97%, at least about 98%, at least about 99%, or 100% identical to SEQ ID NO: 583.

Embodiment 103. The polypeptide molecule of embodiment 98, wherein the Fab or CrossFab comprises (i) a light chain sequence that is at least about 80%, at least about 85%, at least about 90%, at least about 95%, at least about 96%, at least about 97%, at least about 98%, at least about 99%, or 100% identical to SEQ ID NO: 236; and (ii) a heavy chain sequence that is at least about 80%, at least about 85%, at least about 90%, at least about 95%, at least about 96%, at least about 97%, at least about 98%, at least about 99%, or 100% identical to SEQ ID NO: 585.

Embodiment 104. The polypeptide molecule of embodiment 98, wherein the Fab or CrossFab comprises (i) a light chain sequence that is at least about 80%, at least about 85%, at least about 90%, at least about 95%, at least about 96%, at least about 97%, at least about 98%, at least about 99%, or 100% identical to SEQ ID NO: 239; and (ii) a heavy chain sequence that is at least about 80%, at least about 85%, at least about 90%, at least about 95%, at least about 96%, at least about 97%, at least about 98%, at least about 99%, or 100% identical to SEQ ID NO: 586.

Embodiment 105. The polypeptide molecule of embodiment 98, wherein the Fab or CrossFab comprises (i) a light chain sequence that is at least about 80%, at least about 85%, at least about 90%, at least about 95%, at least about 96%, at least about 97%, at least about 98%, at least about 99%, or 100% identical to SEQ ID NO: 240; and (ii) a heavy chain sequence that is at least about 80%, at least about 85%, at least about 90%, at least about 95%, at least about 96%, at least about 97%, at least about 98%, at least about 99%, or 100% identical to SEQ ID NO: 587.

Embodiment 106. The polypeptide molecule of embodiment 98, wherein the Fab or CrossFab comprises (i) a light chain sequence that is at least about 80%, at least about 85%, at least about 90%, at least about 95%, at least about 96%, at least about 97%, at least about 98%, at least about 99%, or 100% identical to SEQ ID NO: 241; and (ii) a heavy chain sequence that is at least about 80%, at least about 85%, at least about 90%, at least about 95%, at least about 96%, at least about 97%, at least about 98%, at least about 99%, or 100% identical to SEQ ID NO: 583.

Embodiment 107. The polypeptide molecule of any one of embodiments 93-97, wherein the Fab or CrossFab comprises (i) a light chain sequence that is at least about 80%, at least about 85%, at least about 90%, at least about 95%, at least about 96%, at least about 97%, at least about 98%, at least about 99%, or 100% identical to any one of SEQ ID NOs: 1280-1292; and (ii) a heavy chain sequence that is at least about 80%, at least about 85%, at least about 90%, at least about 95%, at least about 96%, at least about 97%, at least about 98%, at least about 99%, or 100% identical to any one of SEQ ID NOs: 1293-1328.

Embodiment 108. The polypeptide molecule of embodiment 107, wherein the Fab or CrossFab comprises (i) a light chain sequence that is at least about 80%, at least about 85%, at least about 90%, at least about 95%, at least about 96%, at least about 97%, at least about 98%, at least about 99%, or 100% identical to SEQ ID NO: 1288; and (ii) a heavy chain sequence that is at least about 80%, at least about 85%, at least about 90%, at least about 95%, at least about 96%, at least about 97%, at least about 98%, at least about 99%, or 100% identical to SEQ ID NO: 1315.

Embodiment 109. The polypeptide molecule of embodiment 107, wherein the Fab or CrossFab comprises (i) a light chain sequence that is at least about 80%, at least about 85%, at least about 90%, at least about 95%, at least about 96%, at least about 97%, at least about 98%, at least about 99%, or 100% identical to SEQ ID NO: 1290; and (ii) a heavy chain sequence that is at least about 80%, at least about 85%, at least about 90%, at least about 95%, at least about 96%, at least about 97%, at least about 98%, at least about 99%, or 100% identical to SEQ ID NO: 1317.

Embodiment 110. The polypeptide molecule of embodiment 107, wherein the Fab or CrossFab comprises (i) a light chain sequence that is at least about 80%, at least about 85%, at least about 90%, at least about 95%, at least about 96%, at least about 97%, at least about 98%, at least about 99%, or 100% identical to SEQ ID NO: 1284; and (ii) a heavy chain sequence that is at least about 80%, at least about 85%, at least about 90%, at least about 95%, at least about 96%, at least about 97%, at least about 98%, at least about 99%, or 100% identical to SEQ ID NO: 1322.

Embodiment 111. The polypeptide molecule of embodiment 107, wherein the Fab or CrossFab comprises (i) a light chain sequence that is at least about 80%, at least about 85%, at least about 90%, at least about 95%, at least about 96%, at least about 97%, at least about 98%, at least about 99%, or 100% identical to SEQ ID NO: 1284; and (ii) a heavy chain sequence that is at least about 80%, at least about 85%, at least about 90%, at least about 95%, at least about 96%, at least about 97%, at least about 98%, at least about 99%, or 100% identical to SEQ ID NO: 1323.

Embodiment 112. The polypeptide molecule of embodiment 107, wherein the Fab or CrossFab comprises (i) a light chain sequence that is at least about 80%, at least about 85%, at least about 90%, at least about 95%, at least about 96%, at least about 97%, at least about 98%, at least about 99%, or 100% identical to SEQ ID NO: 1286; and (ii) a heavy chain sequence that is at least about 80%, at least about 85%, at least about 90%, at least about 95%, at least about 96%, at least about 97%, at least about 98%, at least about 99%, or 100% identical to SEQ ID NO: 1297.

Embodiment 113. The polypeptide molecule of embodiment 107, wherein the Fab or CrossFab comprises (i) a light chain sequence that is at least about 80%, at least about 85%, at least about 90%, at least about 95%, at least about 96%, at least about 97%, at least about 98%, at least about 99%, or 100% identical to SEQ ID NO: 1284; and (ii) a heavy chain sequence that is at least about 80%, at least about 85%, at least about 90%, at least about 95%, at least about 96%, at least about 97%, at least about 98%, at least about 99%, or 100% identical to SEQ ID NO: 1327.

Embodiment 114. The polypeptide molecule of embodiment 107, wherein the Fab or CrossFab comprises (i) a light chain sequence that is at least about 80%, at least about 85%, at least about 90%, at least about 95%, at least about 96%, at least about 97%, at least about 98%, at least about 99%, or 100% identical to SEQ ID NO: 1290; and (ii) a heavy chain sequence that is at least about 80%, at least about 85%, at least about 90%, at least about 95%, at least about 96%, at least about 97%, at least about 98%, at least about 99%, or 100% identical to SEQ ID NO: 1328.

Embodiment 115. A polypeptide molecule comprising a first binding domain and a second binding domain, wherein the first binding domain comprises the TCRβV-binding domain of any one of embodiments 1-114.

Embodiment 116. The polypeptide molecule of embodiment 115, wherein the second binding domain specifically binds to a protein expressed on a B cell.

Embodiment 117. The polypeptide molecule of embodiment 115, wherein the second binding domain specifically binds to a protein expressed on a tumor cell.

Embodiment 118. The polypeptide molecule of embodiment 117, wherein the protein expressed on the tumor cell is a tumor-associated antigen (TAA).

Embodiment 119. The polypeptide molecule of any one of embodiments 115-118, wherein the second binding domain is a TAA-binding domain.

Embodiment 120. The polypeptide molecule of embodiment 119, wherein the TAA-binding domain comprises a PSMA-binding domain.

Embodiment 121. The polypeptide molecule of embodiment 120, wherein the PSMA-binding domain comprises:
a Kabat HCDR1 that comprises the amino acid sequence of RYGMH (SEQ ID NO: 2303); a Kabat HCDR2 that comprises the amino acid sequence of VIWYDGSNKYYADSVKG (SEQ ID NO: 2304); a Kabat HCDR3 that comprises the amino acid sequence of GGDFLYYYYYGMDV (SEQ ID NO: 2305);
a Kabat LCDR1 that comprises the amino acid sequence of RASQGISNYLA (SEQ ID NO: 2306); a Kabat LCDR2 that comprises the amino acid sequence of EASTLQS (SEQ ID NO: 2307); and a Kabat LCDR3 that comprises the amino acid sequence of QNYNSAPFT (SEQ ID NO: 2302).

Embodiment 122. The polypeptide molecule of embodiment 120, wherein the PSMA-binding domain comprises:
an IMGT HCDR1 that comprises the amino acid sequence of GFAFSRYG (SEQ ID NO: 2299); an IMGT HCDR2 that comprises the amino acid sequence of IWYDGSNK (SEQ ID NO: 2300); an IMGT HCDR3 that comprises the amino acid sequence of ARGGDFLYYYYYGMDV (SEQ ID NO: 2301);
an IMGT LCDR1 that comprises the amino acid sequence of QGISNY (SEQ ID NO: 2260); an IMGT LCDR2 that comprises the amino acid sequence of EAS; and an IMGT LCDR3 that comprises the amino acid sequence of QNYNSAPFT (SEQ ID NO: 2302).

Embodiment 123. The polypeptide molecule of embodiment 119, wherein the TAA-binding domain comprises a CD19-binding domain.

Embodiment 124. The polypeptide molecule of embodiment 123, wherein the CD19-binding domain comprises:
a Kabat HCDR1 that comprises the amino acid sequence of SYVMH (SEQ ID NO: 1353); a Kabat HCDR2 that comprises the amino acid sequence of YINPYNDGTKYNEKFQG (SEQ ID NO: 1355); a Kabat HCDR3 that comprises the amino acid sequence of GTYYYGTRVFDY (SEQ ID NO: 1357);
a Kabat LCDR1 that comprises the amino acid sequence of RSSKSLQNVNGNTYLY (SEQ ID NO: 1347); a Kabat LCDR2 that comprises the amino acid sequence of RMSNLNS (SEQ ID NO: 1349); and a Kabat LCDR3 that comprises the amino acid sequence of MQHLEYPIT (SEQ ID NO: 1351).

Embodiment 125. The polypeptide molecule of embodiment 123, wherein the CD19-binding domain comprises:
an IMGT HCDR1 that comprises the amino acid sequence of GYTFTSYV (SEQ ID NO: 1361); an IMGT HCDR2 that comprises the amino acid sequence of INPYNDGT (SEQ ID NO: 1363); an IMGT HCDR3 that comprises the amino acid sequence of ARGTYYYGTRVFDY (SEQ ID NO: 1365);
an IMGT LCDR1 that comprises the amino acid sequence of KSLQNVNGNTY (SEQ ID NO: 1359); an IMGT LCDR2 that comprises the amino acid sequence of RMS; and an IMGT LCDR3 that comprises the amino acid sequence of MQHLEYPIT (SEQ ID NO: 1351).

Embodiment 126. The polypeptide molecule of embodiment 123, wherein the CD19-binding domain comprises:
a Kabat HCDR1 that comprises the amino acid sequence of TSTMGVG (SEQ ID NO: 1354); a Kabat HCDR2 that comprises the amino acid sequence of FIWWDDDKRYNPNLKS (SEQ ID NO: 1356); a Kabat HCDR3 that comprises the amino acid sequence of MELWSYYFDP (SEQ ID NO: 1358);
a Kabat LCDR1 that comprises the amino acid sequence of SASSSVGYMH (SEQ ID NO: 1348); a Kabat LCDR2 that comprises the amino acid sequence of DTSKLAF (SEQ ID NO: 1350); and a Kabat LCDR3 that comprises the amino acid sequence of FQGSVYPFT (SEQ ID NO: 1352).

Embodiment 127. The polypeptide molecule of embodiment 123, wherein the CD19-binding domain comprises:
an IMGT HCDR1 that comprises the amino acid sequence of GGSISTSTMG (SEQ ID NO: 1362); an IMGT HCDR2 that comprises the amino acid sequence of IWWDDDK (SEQ ID NO: 1364); an IMGT HCDR3 that comprises the amino acid sequence of ARMELWSYYFDP (SEQ ID NO: 1366);
an IMGT LCDR1 that comprises the amino acid sequence of SSVGY (SEQ ID NO: 1360); an IMGT LCDR2 that comprises the amino acid sequence of DTS; and an IMGT LCDR3 that comprises the amino acid sequence of FQGSVYPFT (SEQ ID NO: 1352).

Embodiment 128. The polypeptide molecule of any one of embodiments 119-127, wherein the TAA-binding domain comprises an immunoglobulin heavy chain variable (VH) region and/or an immunoglobulin light chain variable (VL) region.

Embodiment 129. The polypeptide molecule of embodiment 128, wherein the VH region comprises an amino acid sequence that is at least about 80%, at least about 85%, at least about 90%, at least about 95%, at least about 96%, at least about 97%, at least about 98%, at least about 99%, or 100% identical to SEQ ID NO: 1345; and the VL region comprises an amino acid sequence that is at least about 80%, at least about 85%, at least about 90%, at least about 95%, at least about 96%, at least about 97%, at least about 98%, at least about 99%, or 100% identical to SEQ ID NO: 1343.

Embodiment 130. The polypeptide molecule of embodiment 128, wherein the VH region comprises an amino acid sequence that is at least about 80%, at least about 85%, at least about 90%, at least about 95%, at least about 96%, at least about 97%, at least about 98%, at least about 99%, or 100% identical to SEQ ID NO: 1346; and the VL region comprises an amino acid sequence that is at least about 80%, at least about 85%, at least about 90%, at least about 95%, at least about 96%, at least about 97%, at least about 98%, at least about 99%, or 100% identical to SEQ ID NO: 1344.

Embodiment 131. The polypeptide molecule of any one of embodiments 119 to 130, wherein the TAA-binding domain is a single chain variable fragment (scFv), Fab, CrossFab, Fab′, or F(ab′)2.

Embodiment 132. The polypeptide molecule of embodiment 131, wherein the TAA-binding domain is an scFv comprising a VH region and a VL region.

Embodiment 133. The polypeptide molecule of embodiment 132, wherein the C-terminus of the VH region is linked to the N-terminus of the VL region.

Embodiment 134. The polypeptide molecule of embodiment 132, wherein the C-terminus of the VL region is linked to the N-terminus of the VH region.

Embodiment 135. The polypeptide molecule of embodiment 134, wherein the VH region and VL region are linked by a peptide linker.

Embodiment 136. The polypeptide molecule of embodiment 135, wherein the peptide linker comprises the formula of (Gly4Ser)ₙ, wherein n=1-5 (SEQ ID NO: 1424).

Embodiment 137. The polypeptide molecule of embodiment 135, wherein the peptide linker is (Gly₄Ser)₃ (SEQ ID NO: 1342).

Embodiment 138. The polypeptide molecule of embodiment 135, wherein the peptide linker is (Gly₄Ser) (SEQ ID NO: 1425).

Embodiment 139. The polypeptide of any one of embodiments 132-138, wherein the VH region and the VL region are linked by one or more engineered disulfide bond(s).

Embodiment 140. The binding molecule of embodiment 139, wherein one cysteine that forms the engineered disulfide bond is located in the VH region, and wherein the other cysteine that forms the engineered disulfide bond is located in the VL region.

Embodiment 141. The binding molecule of embodiment 139 or 140, wherein the positions of the one or more engineered disulfide bond(s) are selected from the group consisting of: VH44-VL100, VH100B-VL49, VH100-VL50, VH101-VL46, and VH105-VL43, according to EU numbering.

Embodiment 142. The polypeptide molecule of embodiment 132, wherein the scFv comprises an amino acid sequence that is at least about 80%, at least about 85%, at least about 90%, at least about 95%, at least about 96%, at least about 97%, at least about 98%, at least about 99%, or 100% identical to SEQ ID NO: 1367.

Embodiment 143. The polypeptide molecule of embodiment 132, wherein the scFv comprises an amino acid sequence of SEQ ID NO: 1367.

Embodiment 144. The polypeptide molecule of embodiment 131, wherein the TAA-binding domain is a Fab comprising the VH region, the VL region, a first heavy chain constant region (CH1), and a light chain constant region (CL), wherein the C-terminus of the VH region is connected to the N-terminus of the CH1 region, and the C-terminus of the VL region is connected to the N-terminus of the CL region.

Embodiment 145. The polypeptide molecule of embodiment 131, wherein the TAA-binding domain is a CrossFab comprising the VH region, the VL region, a first heavy chain constant region (CH1), and a light chain constant region (CL), wherein the C-terminus of the VH region is connected to the N-terminus of the CL region, and the C-terminus of the VL region is connected to the N-terminus of the CH1 region.

Embodiment 146. The polypeptide molecule of embodiment 144 or 145, wherein the CH1 region and the CL region are connected by a disulfide bond.

Embodiment 147. The polypeptide molecule of any one of embodiments 144-146, wherein the TAA-binding domain is a charge steering Fab or charge steering CrossFab.

Embodiment 148. The polypeptide molecule of embodiment 147, wherein the CH1 region of the charge steering Fab or charge steering CrossFab comprises mutations K147E and/or K213E according to EU numbering, and the CL region of the charge steering Fab or charge steering CrossFab comprises mutations E123R and/or Q124K according to EU numbering.

Embodiment 149. The polypeptide molecule of any one of embodiments 144-148, wherein the Fab or CrossFab comprises (i) a light chain sequence that is at least about 80%, at least about 85%, at least about 90%, at least about 95%, at least about 96%, at least about 97%, at least about 98%, at least about 99%, or 100% identical to SEQ ID NO: 1369; and (ii) a heavy chain sequence that is at least about 80%, at least about 85%, at least about 90%, at least about 95%, at least about 96%, at least about 97%, at least about 98%, at least about 99%, or 100% identical to SEQ ID NO: 1371.

Embodiment 150. The polypeptide molecule of any one of embodiments 144-148, wherein the Fab or CrossFab comprises (i) a light chain sequence that is at least about 80%, at least about 85%, at least about 90%, at least about 95%, at least about 96%, at least about 97%, at least about 98%, at least about 99%, or 100% identical to SEQ ID NO: 1368; and (ii) a heavy chain sequence that is at least about 80%, at least about 85%, at least about 90%, at least about 95%, at least about 96%, at least about 97%, at least about 98%, at least about 99%, or 100% identical to SEQ ID NO: 1370.

Embodiment 151. The polypeptide molecule of any one of embodiments 144-148, wherein the Fab or CrossFab comprises (i) a light chain sequence that is at least about 80%, at least about 85%, at least about 90%, at least about 95%, at least about 96%, at least about 97%, at least about 98%, at least about 99%, or 100% identical to SEQ ID NO: 54; and (ii) a heavy chain sequence that is at least about 80%, at least about 85%, at least about 90%, at least about 95%, at least about 96%, at least about 97%, at least about 98%, at least about 99%, or 100% identical to SEQ ID NO: 57.

Embodiment 152. The polypeptide molecule of any one of embodiments 115-151, wherein the first binding domain is an scFv, and wherein the second binding domain is a Fab, a CrossFab, a charge steering Fab, or a charge steering CrossFab.

Embodiment 153. The polypeptide molecule of any one of embodiments 115-151, wherein the first binding domain is a Fab, a CrossFab, a charge steering Fab, or a charge steering CrossFab, and wherein the second binding domain is an scFv.

Embodiment 154. The polypeptide molecule of any one of embodiments 115-153, wherein the first binding domain and the second binding domain are linked by a peptide linker.

Embodiment 155. The polypeptide molecule of embodiment 154, wherein the peptide linker comprises the formula of (Gly₄Ser)ₙ, wherein n=1-5.

Embodiment 156. The polypeptide molecule of embodiment 154, wherein the peptide linker is (Gly₄Ser)₃.

Embodiment 157. The polypeptide molecule of embodiment 154, wherein the peptide linker is (Gly₄Ser)₁.

Embodiment 158. The polypeptide molecule of any one of embodiments 115-157, wherein the polypeptide molecule further comprises a half-life extension moiety.

Embodiment 159. The polypeptide molecule of embodiment 158, wherein the half-life extension moiety is selected from the group consisting of: a fragment crystallizable (Fc) region, albumin, transferrin, a binding domain that specifically binds to transferrin or albumin, an unstructured biodegradable proteins (XTEN), a polyethylene glycol (PEG), a glycine-rich homo-amino-acid polymer (HAP), a hydroxyethyl starch (HES), a polysialic acid (PSA), an immunoglobulin binding polypeptide, and a conformationally disordered polypeptide comprising Proline, Alanine and Serine (PAS), or is a derivative or functional fragment thereof.

Embodiment 160. The polypeptide molecule of embodiment 158 or 159, wherein the half-life extension moiety comprises an amino acid sequence with at least about 80%, at least about 85%, at least about 90%, at least about 95%, at least about 96%, at least about 97%, at least about 98%, at least about 99%, or 100% identity to SEQ ID NO: 2510.

Embodiment 161. The polypeptide molecule of embodiment 158 or 159, wherein the half-life extension moiety comprises an amino acid sequence with at least about 80%, at least about 85%, at least about 90%, at least about 95%, at least about 96%, at least about 97%, at least about 98%, at least about 99%, or 100% identity to SEQ ID NO: 2511.

Embodiment 162. The polypeptide molecule of any one of embodiments 158-161, wherein the half-life extension moiety is connected to the polypeptide molecule by a linker.

Embodiment 163. The polypeptide molecule of embodiment 162, wherein the linker comprises an amino acid sequence selected from the group consisting of SEQ ID NOs: 1342, 1424, 1425, 2339, 2340, 2341, and 2515-2523.

Embodiment 164. The polypeptide molecule of any one of embodiments 115-163, wherein the polypeptide molecule further comprises an Fc region.

Embodiment 165. The polypeptide molecule of embodiment 164, wherein the Fc region comprises two polypeptides.

Embodiment 166. The polypeptide molecule of embodiment 165, wherein the first binding domain is connected to one of the polypeptides of the Fc region via a peptide linker and the second binding domain is connected to the other polypeptide of the Fc region via a peptide linker.

Embodiment 167. The polypeptide molecule of embodiment 165, wherein the first binding domain and the second binding domain are connected to the same polypeptide of the Fc region via a peptide linker.

Embodiment 168. The polypeptide molecule of embodiment 166 or 167, wherein the peptide linker comprises an amino acid sequence having the formula of (Gly₄Ser)ₙ, wherein n=1-5.

Embodiment 169. The polypeptide molecule of embodiment 166 or 167, wherein the peptide linker comprises an amino acid sequence amino acid sequence of KTHTCP (SEQ ID NO: 2515).

Embodiment 170. The polypeptide molecule of embodiment 166 or 167, wherein the peptide linker comprises an amino acid sequence amino acid sequence of DKTHTCP (SEQ ID NO: 2517).

Embodiment 171. The polypeptide molecule of any one of embodiments 165-170, wherein the Fc region has a knobs-into-holes format, wherein the first polypeptide of the Fc region comprises one or more hole mutations, and wherein second polypeptide of the Fc region comprises one or more knob mutations.

Embodiment 172. The polypeptide molecule of embodiment 171, wherein the first polypeptide of the Fc region comprises mutations T366S, L368A and Y407V according to EU numbering, and wherein the second polypeptide of the Fc region comprises a mutation T366W according to EU numbering.

Embodiment 173. The polypeptide molecule of embodiment 171 or 172, wherein the first polypeptide of the Fc region comprising the one or more hole mutations further comprises a mutation Y349C according to EU numbering, wherein the second polypeptide of the Fc region comprising the one or more knob mutations further comprises a mutation S354C according to EU numbering, and wherein the Fc region comprises an engineered disulfide bond formed between these two cysteines.

Embodiment 174. The polypeptide molecule of any one of embodiments 165-173, wherein one or both of the polypeptides of the Fc region comprise mutations L234A, L235A, and P329G according to EU numbering.

Embodiment 175. The polypeptide molecule of any one of embodiments 165-174, wherein one or both of the polypeptides of the Fc region comprise the mutation N297G according to EU numbering.

Embodiment 176. The polypeptide molecule of any one of embodiments 165-175, wherein one or both of the polypeptides of the Fc region comprise mutations H435R and Y436F according to EU numbering.

Embodiment 177. The polypeptide molecule of any one of embodiments 165-176, wherein the Fc region lacks one or more effector functions and/or lacks glycosylation.

Embodiment 178. The polypeptide molecule of embodiment 177, wherein the effector function is selected from the group consisting of antibody-dependent cellular cytotoxicity (ADCC), antibody-dependent cellular phagocytosis (ADCP), complement-dependent cytotoxicity (CDC), and binding to a Fcγ receptor (FcγR), or is any combination thereof.

Embodiment 179. The polypeptide molecule of any one of embodiments 165-174, wherein the Fc region lacks glycine-lysine (GK) residues at the C terminus.

Embodiment 180. The polypeptide molecule of any one of embodiments 165-179, wherein the first polypeptide of the Fc region comprises an amino acid sequence that is at least about 80%, at least about 85%, at least about 90%, at least about 95%, at least about 96%, at least about 97%, at least about 98%, at least about 99%, or 100% identical to SEQ ID NO: 1412, and wherein the second polypeptide of the Fc region comprises an amino acid sequence that is at least about 80%, at least about 85%, at least about 90%, at least about 95%, at least about 97%, at least about 98%, at least about 99%, or 100% identical to SEQ ID NO: 1414.

Embodiment 181. The polypeptide molecule of any one of embodiments 165-179, wherein the first polypeptide of the Fc region comprises an amino acid sequence that is at least about 80%, at least about 85%, at least about 90%, at least about 95%, at least about 96%, at least about 97%, at least about 98%, at least about 99%, or 100% identical to SEQ ID NO: 1413, and wherein the second polypeptide of the Fc region comprises an amino acid sequence that is at least about 80%, at least about 85%, at least about 90%, at least about 95%, at least about 96%, at least about 97%, at least about 98%, at least about 99%, or 100% identical to SEQ ID NO: 1415.

Embodiment 182. The polypeptide molecule of any one of embodiments 115-181, wherein the polypeptide molecule comprises three polypeptides.

Embodiment 183. The polypeptide molecule of embodiment 182, wherein the first polypeptide comprises: (i) a peptide linker; and (ii) an Fc heavy chain comprising a hole mutation; the second polypeptide comprises: (i) an anti-TCRβV scFv; (ii) a peptide linker; and (iii) an anti-CD19 Fab light chain; and the third polypeptide comprises: (i) an anti-CD19 Fab heavy chain; (ii) a peptide linker; and (iii) an Fe heavy chain comprising a knob mutation.

Embodiment 184. The polypeptide molecule of embodiment 182, wherein the first polypeptide comprises: (i) a peptide linker; and (ii) an Fc heavy chain comprising a hole mutation; the second polypeptide comprises: (i) an anti-CD19 Fab light chain; and the third polypeptide comprises: (i) an anti-TCRβV scFv; (ii) a peptide linker; (iii) an anti-CD19 Fab heavy chain; (iv) a peptide linker; and (v) an Fc heavy chain comprising a knob mutation.

Embodiment 185. The polypeptide molecule of embodiment 182, wherein the first polypeptide comprises: (i) a peptide linker; and (ii) an Fc heavy chain comprising a hole mutation; the second polypeptide comprises: (i) an anti-CD19 Fab heavy chain; (ii) a peptide linker; and (iii) an Fc heavy chain comprising a knob mutation; and the third polypeptide comprises: (i) an anti-TCRβV scFv; (ii) a peptide linker; and (iii) an anti-CD19 Fab light chain.

Embodiment 186. The polypeptide molecule of embodiment 182, wherein the first polypeptide comprises: (i) a peptide linker; and (ii) an Fc heavy chain comprising a hole mutation; the second polypeptide comprises: (i) an anti-TCRβV scFv; (ii) a peptide linker; (iii) an anti-CD19 Fab heavy chain; (iv) a peptide linker; and (v) an Fc heavy chain comprising a knob mutation; and the third polypeptide comprises an anti-CD19 Fab light chain.

Embodiment 187. The polypeptide molecule of embodiment 182, wherein the first polypeptide comprises an amino acid sequence that is at least about 80%, at least about 85%, at least about 90%, at least about 95%, at least about 96%, at least about 97%, at least about 98%, at least about 99%, or 100% identical to SEQ ID NO: 1412, the second polypeptide comprises an amino acid sequence that is at least about 80%, at least about 85%, at least about 90%, at least about 95%, at least about 96%, at least about 97%, at least about 98%, at least about 99%, or 100% identical to SEQ ID NO: 1416, and the third polypeptide comprises an amino acid sequence that is at least about 80%, at least about 85%, at least about 90%, at least about 95%, at least about 96%, at least about 97%, at least about 98%, at least about 99%, or 100% identical to SEQ ID NO: 1417.

Embodiment 188. The polypeptide molecule of embodiment 182, wherein the first polypeptide comprises an amino acid sequence that is at least about 80%, at least about 85%, at least about 90%, at least about 95%, at least about 96%, at least about 97%, at least about 98%, at least about 99%, or 100% identical to SEQ ID NO: 1412, the second polypeptide comprises an amino acid sequence that is at least about 80%, at least about 85%, at least about 90%, at least about 95%, at least about 96%, at least about 97%, at least about 98%, at least about 99%, or 100% identical to SEQ ID NO: 1369, and the third polypeptide comprises an amino acid sequence that is at least about 80%, at least about 85%, at least about 90%, at least about 95%, at least about 96%, at least about 97%, at least about 98%, at least about 99%, or 100% identical to SEQ ID NO: 1418.

Embodiment 189. The polypeptide molecule of embodiment 182, wherein the first polypeptide comprises an amino acid sequence that is at least about 80%, at least about 85%, at least about 90%, at least about 95%, at least about 96%, at least about 97%, at least about 98%, at least about 99%, or 100% identical to SEQ ID NO: 1412, the second polypeptide comprises an amino acid sequence that is at least about 80%, at least about 85%, at least about 90%, at least about 95%, at least about 96%, at least about 97%, at least about 98%, at least about 99%, or 100% identical to SEQ ID NO: 1419, and the third polypeptide comprises an amino acid sequence that is at least about 80%, at least about 85%, at least about 90%, at least about 95%, at least about 96%, at least about 97%, at least about 98%, at least about 99%, or 100% identical to SEQ ID NO: 1417.

Embodiment 190. The polypeptide molecule of embodiment 182, wherein the first polypeptide comprises an amino acid sequence that is at least about 80%, at least about 85%, at least about 90%, at least about 95%, at least about 96%, at least about 97%, at least about 98%, at least about 99%, or 100% identical to SEQ ID NO: 1412, the second polypeptide comprises an amino acid sequence that is at least about 80%, at least about 85%, at least about 90%, at least about 95%, at least about 96%, at least about 97%, at least about 98%, at least about 99%, or 100% identical to SEQ ID NO: 1420, and the third polypeptide comprises an amino acid sequence that is at least about 80%, at least about 85%, at least about 90%, at least about 95%, at least about 96%, at least about 97%, at least about 98%, at least about 99%, or 100% identical to SEQ ID NO: 1417.

Embodiment 191. The polypeptide molecule of embodiment 182, wherein the first polypeptide comprises an amino acid sequence that is at least about 80%, at least about 85%, at least about 90%, at least about 95%, at least about 96%, at least about 97%, at least about 98%, at least about 99%, or 100% identical to SEQ ID NO: 1412, the second polypeptide comprises an amino acid sequence that is at least about 80%, at least about 85%, at least about 90%, at least about 95%, at least about 96%, at least about 97%, at least about 98%, at least about 99%, or 100% identical to SEQ ID NO: 1369, and the third polypeptide comprises an amino acid sequence that is at least about 80%, at least about 85%, at least about 90%, at least about 95%, at least about 96%, at least about 97%, at least about 98%, at least about 99%, or 100% identical to SEQ ID NO: 1421.

Embodiment 192. The polypeptide molecule of embodiment 182, wherein the first polypeptide comprises an amino acid sequence that is at least about 80%, at least about 85%, at least about 90%, at least about 95%, at least about 96%, at least about 97%, at least about 98%, at least about 99%, or 100% identical to SEQ ID NO: 1412, the second polypeptide comprises an amino acid sequence that is at least about 80%, at least about 85%, at least about 90%, at least about 95%, at least about 96%, at least about 97%, at least about 98%, at least about 99%, or 100% identical to SEQ ID NO: 1369, and the third polypeptide comprises an amino acid sequence that is at least about 80%, at least about 85%, at least about 90%, at least about 95%, at least about 96%, at least about 97%, at least about 98%, at least about 99%, or 100% identical to SEQ ID NO: 1422.

Embodiment 193. The polypeptide molecule of embodiment 182, wherein the first polypeptide comprises an amino acid sequence that is at least about 80%, at least about 85%, at least about 90%, at least about 95%, at least about 96%, at least about 97%, at least about 98%, at least about 99%, or 100% identical to SEQ ID NO: 1412, the second polypeptide comprises an amino acid sequence that is at least about 80%, at least about 85%, at least about 90%, at least about 95%, at least about 96%, at least about 97%, at least about 98%, at least about 99%, or 100% identical to SEQ ID NO: 1369, and the third polypeptide comprises an amino acid sequence that is at least about 80%, at least about 85%, at least about 90%, at least about 95%, at least about 96%, at least about 97%, at least about 98%, at least about 99%, or 100% identical to SEQ ID NO: 1423.

Embodiment 194. A recombinant nucleic acid molecule encoding the polypeptide molecule of any one of embodiments [00913]-193.

Embodiment 195. A vector comprising the recombinant nucleic acid molecule of embodiment 194.

Embodiment 196. A composition comprising the polypeptide molecule of any one of embodiments 1-193, the recombinant nucleic acid molecule of embodiment 194, or the vector of embodiment 195.

Embodiment 197. A pharmaceutical composition comprising (i) the polypeptide molecule of any one of embodiments 1-193, the recombinant nucleic acid molecule of claim 194, or the vector of claim 195, and (ii) a pharmaceutically acceptable carrier, diluent, or excipient.

Embodiment 198. A method of producing a polypeptide, comprising introducing the recombinant nucleic acid molecule of embodiment 194, or the vector of embodiment 195, into a host cell under conditions that express the polypeptide molecule in the cell, wherein the method is ex vivo.

Embodiment 199. A method of expanding a T cell population, wherein the method comprises contacting the T cell population with the polypeptide molecule of any one of embodiments [00913] to 193, or the pharmaceutical composition of embodiment 197, wherein the T cell population expresses a TCRβV antigen on the cell surface, and wherein the polypeptide molecule specifically binds to the TCRβV antigen.

Embodiment 200. The method of embodiment 199, wherein the method expands T cells in vitro or ex vivo.

Embodiment 201. The method of embodiment 199, wherein the method expands T cells in vivo in a subject.

Embodiment 202. The method of any one of embodiments 199-201, wherein the population of T cells comprises a plurality of effector T cells and/or memory T cells.

Embodiment 203. The method of embodiment 202, wherein the effector T cells are selected from the group consisting of effector memory T cells (T_{EM}), T effector cells (T_{EFF}), and T effector cells re-expressing CD45RA (T_{EMRA}), or are a combination thereof.

Embodiment 204. The method of embodiment 202 or 203, wherein the memory T cells are selected from the group consisting of central memory T cells (T_{CM}) and T memory stem cells (T_{SCM}), or are a combination thereof.

Embodiment 205. The method of any one of embodiments 199-204, wherein the population of T cells has an increased expansion of the effector T cells and/or memory T cells than that produced by a polypeptide molecule comprising a CD3-binding domain.

Embodiment 206. The method of any one of embodiments 199-205, wherein the population of T cells has an increased percentage of effector T cells and/or memory T cells than that produced by a polypeptide molecule comprising a CD3-binding domain.

Embodiment 207. The method of embodiment 205 or 206, wherein the effector T cells are effector memory T cells (T_{EM}).

Embodiment 208. The method of embodiment 205 or 206, wherein the memory T cells are central memory T cells (T_{CM}).

Embodiment 209. The method of any one of embodiments 199-208, wherein the expanded T cell population has an increased and/or prolonged cytotoxicity than that of a polypeptide molecule comprising a CD3-binding domain.

Embodiment 210. A method of depleting target cells in a subject, comprising administering to the subject an effective amount of the polypeptide molecule of any one of embodiments 1-193, or the pharmaceutical composition of embodiment 197, wherein the target cells are tumor cells.

Embodiment 211. A method of depleting target cells in a subject, comprising administering to the subject an effective amount of the polypeptide molecule of any one of embodiments 1-193, or the pharmaceutical composition of embodiment 197, wherein the target cells are B cells.

Embodiment 212. The method of embodiment 210 or 211, wherein the method decreases the number of target cells by at least about 50%, at least about 55%, at least about 60%, at least about 65%, at least about 70%, at least about 75%, at least about 80%, at least about 85%, at least about 90%, at least about 95%, at least about 98%, at least about 99%, at least about 99.5%, at least about 99.8%, at least about 99.9%, or 100% in the subject.

Embodiment 213. A method of treating a disease, disorder, or condition in a subject in need thereof, comprising administering to the subject an effective amount of the polypeptide molecule of any one of embodiments 1-193, or the pharmaceutical composition of embodiment 197.

Embodiment 214. The method of embodiment 213, wherein the disease, disorder, or condition is a cancer.

Embodiment 215. The method of embodiment 214, wherein the cancer is a blood cancer.

Embodiment 216. The method of embodiment 215, wherein the blood cancer is chronic lymphocytic leukemia or non-Hodgkin lymphoma.

Embodiment 217. The method of embodiment 214, wherein the cancer is a solid tumor.

Embodiment 218. The method of embodiment 213, wherein the disease, disorder, or condition is an autoimmune disease.

Embodiment 219. The method of embodiment 218, wherein the autoimmune disease is rheumatoid arthritis, systemic lupus erythematosus, or myasthenia gravis.

Embodiment 220. The method of any one of embodiments 213-219, wherein the subject has previously been treated with a polypeptide molecule comprising a CD3-binding domain.

Embodiment 221. The method of any one of embodiments 213-220, wherein the subject is relapsing or refractory to a treatment comprising a polypeptide molecule comprising a CD3-binding domain.

### Section 2.

Embodiment 1. A polypeptide molecule comprising an anti-T cell receptor beta variable region (TCRβV)-binding domain, wherein the binding domain comprises:
a Kabat heavy chain CDR1 (HCDR1) that comprises the amino acid sequence of X₃₁X₃₂YIH, wherein X₃₁ is selected from the group consisting of: T, L, H, Y, Q, K, F, S, A, D, V, I, R, W, E, G, N, and P and X₃₂ is selected from the group consisting of Y, F, M, W, L, R, T, G, I, V, A, E, Q, P, H, D, N, and K;
a Kabat heavy chain CDR2 (HCDR2) that comprises the amino acid sequence of X₅₀X₅₁X₅₂X₅₃X₅₄X₅₅X₅₆X₅₇X₅₈X₅₉Y₆₀X₆₁X₆₂K₆₃F₆₄K₆₅G₆₆, wherein X₅₀ is selected from the group consisting of: W, E, D, R, V, H, G, Q, S, M, N, Y, I, L, A, T, P, and F; X₅₁ is selected from the group consisting of F, E, H, M, K, Q, Y, L, N, V, T, A, R, G, S, D, W, I, and P; X₅₂ is selected from the group consisting of: F, Y, R, S, A, K, W, N, M, H, G, Q, D, P, V, L, E, T, and I; X₅₃ is selected from the group consisting of: P, G, N, A, I, V, L, T, Y, S, Q, K, F, R, D, E, and W; X₅₄ is selected from the group consisting of G, R, K, W, L, F, Y, Q, H, I, M, S, D, P, T, and V; X₅₅ is selected from the group consisting of S, T, G, W, H, N, Y, L, A, F, Q, K, V, M, D, E, R, and P; X₅₆ is selected from the group consisting of: G, Q, R, S, W, A, H, M, N, Y, E, L, T, D, K, I, V, and P; X₅₇ is selected from the group consisting of N, K, R, T, G, H, V, Y, M, L, Q, I, D, A, E, F, W, S, and P; X₅₈ is selected from the group consisting of: I, V, Y, L, A, Q, K, T, M, E, S, N, R, F, D, P, and W; X₅₉ is selected from the group consisting of: K, N, Q, M, L, H, F, S, A, V, T, W, E, G, P, D, and R; X₆₁ is selected from the group consisting of N, V, S, H, P, T, D, E, W, G, M, Q, F, K, I, Y, and L; and X₆₂ is selected from the group consisting of E, P, Q, I, N, W, H, F, K, A, V, Y, M, L, S, D, R, T, and G;
a Kabat heavy chain CDR3 (HCDR3) that comprises the amino acid sequence of X₉₉X₁₀₀X₁₀₁X₁₀₂X₁₀₃X₁₀₄X₁₀₅X₁₀₆X₁₀₇X₁₀₈, wherein X₉₉ is selected from the group consisting of S, A, P, K, D, E, H, G, L, W, V, Y, I, N, M, R, and F; X₁₀₀ is selected from the group consisting of Y, V, F, I, L, M, T, W, H, D, Q, A, S, E, K, P, N, and R; X₁₀₁ is selected from the group consisting of: Y, W, F, V, H, M, P, Q, S, T, E, N, L, K, D, I, and A; X₁₀₂ is selected from the group consisting of S, A, T, G, N, L, M, V, Y, H, Q, W, I, D, R, E, and F; X₁₀₃ is selected from the group consisting of: Y, F, P, M, K, W, S, V, H, L, R, D, A, E, I, Q, and N; X₁₀₄ is selected from the group consisting of D, N, S, G, L, T, K, Q, H, P, R, E, W, F, and V; X₁₀₅ is selected from the group consisting of V, A, N, Q, T, W, E, P, Y, M, K, H, D, S, F, G, L, and R; X₁₀₆ is selected from the group consisting of: L, I, M, V, Y, A, P, R, T, Q, H, G, K, N, E, D, and S; X₁₀₇ is selected from the group consisting of: D, V, S, Q, I, H, E, R, T, M, Y, G, N, A, F, P, K, W, and L; and X₁₀s is selected from the group consisting of: Y, D, V, S, Q, I, H, E, R, T, M, G, N, A, F, P, K, W, and L;
a Kabat light chain CDR1 (LCDR1) that comprises the amino acid sequence of K₂₄A₂₅S₂₆Q₂₇Z₂₈Z₂₉Z₃₀Z₃₁Z₃₂Z₃₃Z₃₄, wherein Z₂₈ is selected from the group consisting of: N, H, P, T, S, M, G, A, V, W, D, I, F, R, Y, Q, E, and L; Z₂₉ is selected from the group consisting of V, G, A, L, M, S, P, F, Q, T, D, K, W, Y, H, R, and E; Z₃₀ is selected from the group consisting of: G, W, A, L, Q, N, R, M, P, D, K, H, I, V, S, E, and F; Z₃₁ is selected from the group consisting of I, E, Q, G, H, S, W, Y, M, N, P, F, A, V, K, D, and L; Z₃₂ is selected from the group consisting of: N, S, H, A, K, Q, L, D, M, T, E, Y, V, F, W, R, and P; Z₃₃ is selected from the group consisting of V, D, E, N, L, G, A, T, S, Q, H, F, R, K, W, P, and Y; and Z₃₄ is selected from the group consisting of: V, T, N, I, K, P, H, M, F, Q, G, Y, R, W, L, D, A, and S;
a Kabat light chain CDR2 (LCDR2) that comprises the amino acid sequence of Z₅₀SZ₅₂Z₅₃Z₅₄Z₅₅Z₅₆, wherein Z₅₀ is selected from the group consisting of: S, V, A, G, H, L, T, M, F, Q, E, P, K, W, and Y; Z₅₂ is selected from the group consisting of S, W, Y, G, T, M, F, N, L, A, V, Q, I, E, D, H, P, and K; Z₅₃ is selected from the group consisting of H, M, A, Y, L, N, E, V, G, Q, F, K, P, W, and R; Z₅₄ is selected from the group consisting of: R, V, N, P, K, T, W, A, M, L, I, H, Q, F, and D; Z₅₅ is selected from the group consisting of Y, W, M, F, A, S, E, G, V, P, D, N, R, Q, T, and H; and Z₅₆ is selected from the group consisting of: S, R, K, Q, Y, W, F, H, I, V, T, C, N, G, D, M and P;
a Kabat light chain CDR3 (LCDR3) that comprises the amino acid sequence of Z₈₉Z₉₀Z₉₁Z₉₂Z₉₃Z₉₄Z₉₅Z₉₆Z₉₇, wherein Z₈₉ is selected from the group consisting of: Q, Y, K, E, V, A, N, I, G, W, S, R, L, and H; Z₉₀ is selected from the group consisting of Q, M, H, E, N, L, D, G, V, R, I, Y, W, K, S and P; Z₉₁ is selected from the group consisting of F, S, T, A, M, H, L, E, Y, W, N, R, Q, I, P, V, G, K, and D; Z₉₂ is selected from the group consisting of: K, G, I, R, S, V, Y, F, W, M, A, Q, T, H, E, L, D, N, and P; Z₉₃ is selected from the group consisting of S, F, R, A, L, M, K, D, W, N, V, Q, T, I, and G; Z₉₄ is selected from the group consisting of: Y, L, V, I, A, N, Q, R, E, G, P, S, K, D, W, and F; Z₉₅ is selected from the group consisting of P, N, A, G, S, H, V, D, K, T, L, E, M, Q, R, F, and W; Z₉₆ is selected from the group consisting of L, I, V, M, E, Y, F, N, A, G, H, R, S, W, Q, D, P, and T; and Z₉₇ is selected from the group consisting of: T, G, A, S, R, Q, V, W, E, L, I, H, M, F, N, D, and P.
wherein the polypeptide molecule does not comprise SEQ ID NOs: 69-73, 112, and 2416-2457.

Embodiment 2. The polypeptide molecule of embodiment 1, wherein X₃₁ is selected from the group consisting of T and L, or X₃₂ is selected from the group consisting of Y, F, M, W, L, R, and T.

Embodiment 3. The polypeptide molecule of embodiment 1 or embodiment 2, wherein X₃₁ is L.

Embodiment 4. The polypeptide molecule of any one of embodiments 1-3, wherein X₃₂ is selected from the group consisting of M, L, and R.

Embodiment 5. The polypeptide molecule of any one of embodiments 1-4, wherein X₅₀ is W; X₅₁ is F; X₅₂ is F; X₅₃ is P; X₅₄ is selected from the group consisting of G and R; X₅₅ is S; X₅₆ is G; X₅₇ is N; X₅₈ is selected from the group consisting of I, V, Y, L, A, Q, K, T, and M; X₅₉ is K; X₆₁ is selected from the group consisting of N, V, S, H, P, T, and D; or X₆₂ is E.

Embodiment 6. The polypeptide molecule of any one of embodiments 1-5, wherein X₅₄ is R; X₅₈ is selected from the group consisting of L, A, Q, K, and M; or X₆₁ is selected from the group consisting of V, S, H, P, T, and D.

Embodiment 7. The polypeptide molecule of any one of embodiments 1-6, wherein X₅₄ is R.

Embodiment 8. The polypeptide molecule of any one of embodiments 1-7, wherein X₉₉ is S; X₁₀₀ is Y; X₁₀₁ is Y; X₁₀₂ is S; X₁₀₃ is selected from the group consisting of Y and F; X₁₀₄ is D; X₁₀₅ is V; X₁₀₆ is L; X₁₀₇ is D; or X₁₀₈ is Y.

Embodiment 9. The polypeptide molecule of any one of embodiments 1-8, wherein X₁₀₃ is F.

Embodiment 10. The polypeptide molecule of any one of embodiments 1-9, wherein Z₂₈ is N; Z₂₉ is selected from the group consisting of V and G; Z₃₀ is G; Z₃₁ is I; Z₃₂ is selected from the group consisting of N and R; Z₃₃ is selected from the group consisting of V and D; or Z₃₄ is V.

Embodiment 11. The polypeptide molecule of any one of embodiments 1-10, wherein Z₂₉ is G or Z₃₃ is D.

Embodiment 12. The polypeptide molecule of any one of embodiments 1-10, wherein Z₃₂ is R.

Embodiment 13. The polypeptide molecule of any one of embodiments 1-12, wherein Z₅₀ is S; Z₅₂ is S; Z₅₃ is H; Z₅₄ is R; Z₅₅ is Y; or Z₅₆ is selected from the group consisting of S and R.

Embodiment 14. The polypeptide molecule of any one of embodiments 1-13, wherein Z₅₆ is R.

Embodiment 15. The polypeptide molecule of any one of embodiments 1-14, wherein Z₈₉ is Q; Z₉₀ is Q; Z₉₁ is F; Z₉₂ is K; Z₉₃ is selected from the group consisting of S, F, R, A, and L; Z₉₄ is Y; Z₉₅ is selected from the group consisting of P and N; Z₉₆ is selected from the group consisting of L and I; or Z₉₇ is selected from the group consisting of T, G, and A.

Embodiment 16. The polypeptide molecule of any one of embodiments 1-15, wherein Z₉₃ is selected from the group consisting of F, R, A, and L; Z₉₅ is N; Z₉₆ is I; or Z₉₇ is selected from the group consisting of G and A.

Embodiment 17. The polypeptide molecule of any one of embodiments 1-16, wherein Z₉₆ is I.

Embodiment 18. A polypeptide molecule comprising an anti-TCRβV-binding domain, wherein the binding domain comprises:
a Kabat HCDR1 that comprises the amino acid sequence of X₃₁X₃₂YIH, wherein X₃₁ is selected from the group consisting of: T and L; and X₃₂ is selected from the group consisting of Y, F, M, W, L, R, and T;
a Kabat HCDR2 that comprises the amino acid sequence of W₅₀F₅₁F₅₂P₅₃X₅₄S₅₅G₅₆N₅₇X₅₈K₅₉Y₆₀X₆₁E₆₂K₆₃F₆₄K₆₅G₆₆, wherein X₅₄ is selected from the group consisting of G and R; X₅₈ is selected from the group consisting of: I, V, Y, L, A, Q, K, T, and M; and X₆₁ is selected from the group consisting of N, V, S, H, P, T, and D;
a Kabat HCDR3 that comprises the amino acid sequence of S₉₉Y₁₀₀Y₁₀₁S₁₀₂F₁₀₃D₁₀₄V₁₀₅L₁₀₆D₁₀₇Y₁₀₈;
a Kabat LCDR1 that comprises the amino acid sequence of K₂₄A₂₅S₂₆Q₂₇N₂₈Z₂₉G₃₀I₃₁Z₃₂Z₃₃V₃₄, wherein Z₂₉ is selected from the group consisting of V and G; Z₃₂ is selected from the group consisting of N and R; and Z₃₃ is selected from the group consisting of V and D;
a Kabat LCDR2 that comprises the amino acid sequence of S₅₀S51S₅₂H₅₃R₅₄Y₅₅Z₅₆, wherein Z₅₆ is selected from the group consisting of: S and R;
a Kabat light chain CDR3 (LCDR3) that comprises the amino acid sequence of Q₈₉Q₉₀F₉₁K₉₂Z₉₃Y₉₄Z₉₅L₉₆Z₉₇, wherein Z₉₃ is selected from the group consisting of S, F, R, A, and L; Z₉₅ is selected from the group consisting of P and N; Z₉₆ is selected from the group consisting of L and I; and Z₉₇ is selected from the group consisting of T, G, and A.
wherein the polypeptide molecule does not comprise SEQ ID NOs: 69-73, 112, and 2416-2457.

Embodiment 19. A polypeptide molecule comprising an anti-TCRβV-binding domain, wherein the binding comprises:
a Kabat HCDR1 that comprises an amino acid sequence selected from any one of: TYYIH (SEQ ID NO: 112), LYYIH (SEQ ID NO: 136), TWYIH (SEQ ID NO: 145), and LWYIH (SEQ ID NO: 147);
a Kabat HCDR2 that comprises an amino acid sequence selected from any one of: WFFPGSGNIKYNEKFKG (SEQ ID NO: 69) and WFFPRSGNIKYNEKFKG (SEQ ID NO: 142)
a Kabat HCDR3 that comprises an amino acid sequence of SYYSFDVLDY (SEQ ID NO: 137)
a Kabat LCDR1 that comprises an amino acid sequence selected from any one of: KASQNVGINVV (SEQ ID NO: 71) and KASQNVGIRVV (SEQ ID NO: 134);
a Kabat LCDR2 that comprises an amino acid sequence selected from any one of: SSSHRYS (SEQ ID NO: 72), SSSHRYK (SEQ ID NO: 128), and SSSHRYR (SEQ ID NO: 144);
a Kabat LCDR3 that comprises an amino acid sequence selected from any one of: QQFKSYPIT (SEQ ID NO: 135) and QQFKFYPIT (SEQ ID NO: 141);
wherein each of the HCDR1, HCDR2, HCDR3, LCDR1, LCDR2, and/or LCDR3 independently comprise 0, 1, 2, or 3 amino acid mutations.

Embodiment 20. A polypeptide molecule comprising an anti-TCRβV-binding domain, wherein the binding comprises:
a Kabat HCDR1 that comprises an amino acid sequence selected from any one of: TYYIH (SEQ ID NO: 112), LYYIH (SEQ ID NO: 136), TWYIH (SEQ ID NO: 145), and LWYIH (SEQ ID NO: 147);
a Kabat HCDR2 that comprises an amino acid sequence selected from any one of: WFFPGSGNIKYNEKFKG (SEQ ID NO: 69) and WFFPRSGNIKYNEKFKG (SEQ ID NO: 142)
a Kabat HCDR3 that comprises an amino acid sequence of SYYSFDVLDY (SEQ ID NO: 137)
a Kabat LCDR1 that comprises an amino acid sequence selected from any one of: KASQNVGINVV (SEQ ID NO: 71) and KASQNVGIRVV (SEQ ID NO: 134);
a Kabat LCDR2 that comprises an amino acid sequence selected from any one of: SSSHRYS (SEQ ID NO: 72), SSSHRYK (SEQ ID NO: 128), and SSSHRYR (SEQ ID NO: 144);
a Kabat LCDR3 that comprises an amino acid sequence selected from any one of: QQFKSYPIT (SEQ ID NO: 135) and QQFKFYPIT (SEQ ID NO: 141).

Embodiment 21. A polypeptide molecule comprising an anti-TCRβV binding domain, wherein the binding domain comprises:
(i) the HCDR1, the HCDR2, and the HCDR3 comprising the amino acid sequences of SEQ ID NOs: 136, 69, and 137, respectively, and the LCDR1, the LCDR2, and the LCDR3 comprising the amino acid sequences of SEQ ID NOs: 134, 72, and 135, respectively;
(ii) the HCDR1, the HCDR2, and the HCDR3 comprising the amino acid sequences of SEQ ID NOs: 112, 142, and 137, respectively, and the LCDR1, the LCDR2, and the LCDR3 comprising the amino acid sequences of SEQ ID NOs: 134, 72, and 141, respectively;
(iii) the HCDR1, the HCDR2, and the HCDR3 comprising the amino acid sequences of SEQ ID NOs: 112, 69, and 137, respectively, and the LCDR1, the LCDR2, and the LCDR3 comprising the amino acid sequences of SEQ ID NOs: 134, 144, and 135, respectively;
(iv) the HCDR1, the HCDR2, and the HCDR3 comprising the amino acid sequences of SEQ ID NOs: 112, 142, and 137, respectively, and the LCDR1, the LCDR2, and the LCDR3 comprising the amino acid sequences of SEQ ID NOs: 134, 72, and 135, respectively;
(v) the HCDR1, the HCDR2, and the HCDR3 comprising the amino acid sequences of SEQ ID NOs: 147, 69, and 137, respectively, and the LCDR1, the LCDR2, and the LCDR3 comprising the amino acid sequences of SEQ ID NOs: 134, 72, and 135, respectively;
(vi) the HCDR1, the HCDR2, and the HCDR3 comprising the amino acid sequences of SEQ ID NOs: 147, 142, and 137, respectively, and the LCDR1, the LCDR2, and the LCDR3 comprising the amino acid sequences of SEQ ID NOs: 71, 128, and 135, respectively;
(vii) the HCDR1, the HCDR2, and the HCDR3 comprising the amino acid sequences of SEQ ID NOs: 136, 142, and 137, respectively, and the LCDR1, the LCDR2, and the LCDR3 comprising the amino acid sequences of SEQ ID NOs: 134, 128, and 135, respectively;
(viii) the HCDR1, the HCDR2, and the HCDR3 comprising the amino acid sequences of SEQ ID NOs: 112, 142, and 137, respectively, and the LCDR1, the LCDR2, and the LCDR3 comprising the amino acid sequences of SEQ ID NOs: 71, 144, and 135, respectively;
(ix) the HCDR1, the HCDR2, and the HCDR3 comprising the amino acid sequences of SEQ ID NOs: 112, 142, and 137, respectively, and the LCDR1, the LCDR2, and the LCDR3 comprising the amino acid sequences of SEQ ID NOs: 134, 144, and 135, respectively;
(x) the HCDR1, the HCDR2, and the HCDR3 comprising the amino acid sequences of SEQ ID NOs: 136, 142, and 137, respectively, and the LCDR1, the LCDR2, and the LCDR3 comprising the amino acid sequences of SEQ ID NOs: 134, 144, and 135, respectively;
(xi) the HCDR1, the HCDR2, and the HCDR3 comprising the amino acid sequences of SEQ ID NOs: 145, 69, and 137, respectively, and the LCDR1, the LCDR2, and the LCDR3 comprising the amino acid sequences of SEQ ID NOs: 134, 128, and 141, respectively;
(xii) the HCDR1, the HCDR2, and the HCDR3 comprising the amino acid sequences of SEQ ID NOs: 147, 69, and 137, respectively, and the LCDR1, the LCDR2, and the LCDR3 comprising the amino acid sequences of SEQ ID NOs: 71, 144, and 141, respectively;
(xiii) the HCDR1, the HCDR2, and the HCDR3 comprising the amino acid sequences of SEQ ID NOs: 147, 69, and 137, respectively, and the LCDR1, the LCDR2, and the LCDR3 comprising the amino acid sequences of SEQ ID NOs: 134, 72, and 135, respectively;
(xiv) the HCDR1, the HCDR2, and the HCDR3 comprising the amino acid sequences of SEQ ID NOs: 112, 142, and 137, respectively, and the LCDR1, the LCDR2, and the LCDR3 comprising the amino acid sequences of SEQ ID NOs: 134, 128, and 141, respectively;
(xv) the HCDR1, the HCDR2, and the HCDR3 comprising the amino acid sequences of SEQ ID NOs: 136, 69, and 137, respectively, and the LCDR1, the LCDR2, and the LCDR3 comprising the amino acid sequences of SEQ ID NOs: 134, 128, and 141, respectively;
(xvi) the HCDR1, the HCDR2, and the HCDR3 comprising the amino acid sequences of SEQ ID NOs: 147, 142, and 137, respectively, and the LCDR1, the LCDR2, and the LCDR3 comprising the amino acid sequences of SEQ ID NOs: 134, 144, and 135, respectively;
(xvii) the HCDR1, the HCDR2, and the HCDR3 comprising the amino acid sequences of SEQ ID NOs: 136, 142, and 137, respectively, and the LCDR1, the LCDR2, and the LCDR3 comprising the amino acid sequences of SEQ ID NOs: 71, 144, and 135, respectively; or
(xviii) the HCDR1, the HCDR2, and the HCDR3 comprising the amino acid sequences of SEQ ID NOs: 147, 142, and 137, respectively, and the LCDR1, the LCDR2, and the LCDR3 comprising the amino acid sequences of SEQ ID NOs: 134, 144, and 141, respectively.

Embodiment 22. A polypeptide molecule comprising an anti-TCRβV-binding domain, wherein the binding domain comprises:
an IMGT HCDR1 that comprises the amino acid sequence of G₂₆X₂₇X₂₈X₂₉X₃₀X₃₁X₃₂Y₃₃, wherein X₂₇ is selected from the group consisting of: Y, P, E, H, V, K, S, Q, T, L, N, F, M, I, R, G, W, and D; X₂₈ is selected from the group consisting of S, K, A, P, T, V, N, Q, L, E, I, H, M, G, Y, and W; X₂₉ is selected from the group consisting of: F, I, M, Q, L, W, P, V, S, Y, N, A, D, T, E, H, K, G, and R; X₃₀ is selected from the group consisting of T, V, L, Y, W, Q, I, E, R, G, N, F, K, M, S, H, A, and P; X₃₁ is selected from the group consisting of: T, L, H, Y, Q, K, F, S, A, D, V, I, R, W, E, G, N, and P; and X₃₂ is selected from the group consisting of Y, F, M, W, L, R, T, G, I, V, A, E, Q, P, H, D, N, and K;
an IMGT HCDR2 that comprises the amino acid sequence of X₅₁X₅₂X₅₃X₅₄X₅₅X₅₆X₅₇X₅₈, wherein X₅₁ is selected from the group consisting of F, E, H, M, K, Q, Y, L, N, V, T, A, R, G, S, D, W, I, and P; X₅₂ is selected from the group consisting of: F, Y, R, S, A, K, W, N, M, H, G, Q, D, P, V, L, E, T, and I; X₅₃ is selected from the group consisting of: P, G, N, A, I, V, L, T, Y, S, Q, K, F, R, D, E, and W; X₅₄ is selected from the group consisting of G, R, K, W, L, F, Y, Q, H, I, M, S, D, P, T, and V; X₅₅ is selected from the group consisting of: S, T, G, W, H, N, Y, L, A, F, Q, K, V, M, D, E, R, and P; X₅₆ is selected from the group consisting of: G, Q, R, S, W, A, H, M, N, Y, E, L, T, D, K, I, V, and P; X₅₇ is selected from the group consisting of N, K, R, T, G, H, V, Y, M, L, Q, I, D, A, E, F, W, S, and P; and X₅₈ is selected from the group consisting of: I, V, Y, L, A, Q, K, T, M, E, S, N, R, F, D, P, and W;
an IMGT HCDR3 that comprises the amino acid sequence of A₉₇X₉₈X₉₉X₁₀₀X₁₀₁X₁₀₂X₁₀₃X₁₀₄X₁₀₅X₁₀₆X₁₀₇X₁₀₈, wherein X₉₈ is selected from the group consisting of: G, A, V, P, M, I, D, Q, N, F, L, W, C, and R; X₉₉ is selected from the group consisting of S, A, P, K, D, E, H, G, L, W, V, Y, I, N, M, R, and F; X₁₀₀ is selected from the group consisting of: Y, V, F, I, L, M, T, W, H, D, Q, A, S, E, K, P, N, and R; X₁₀₁ is selected from the group consisting of: Y, W, F, V, H, M, P, Q, S, T, E, N, L, K, D, I, and A; X₁₀₂ is selected from the group consisting of S, A, T, G, N, L, M, V, Y, H, Q, W, I, D, R, E, and F; X₁₀₃ is selected from the group consisting of: Y, F, P, M, K, W, S, V, H, L, R, D, A, E, I, Q, and N; X₁₀₄ is selected from the group consisting of D, N, S, G, L, T, K, Q, H, P, R, E, W, F, and V; X₁₀₅ is selected from the group consisting of V, A, N, Q, T, W, E, P, Y, M, K, H, D, S, F, G, L, and R; X₁₀₆ is selected from the group consisting of: L, I, M, V, Y, A, P, R, T, Q, H, G, K, N, E, D, and S; X₁₀₇ is selected from the group consisting of: D, V, S, Q, I, H, E, R, T, M, Y, G, N, A, F, P, K, W, and L; and X₁₀₈ is selected from the group consisting of: Y, D, V, S, Q, I, H, E, R, T, M, G, N, A, F, P, K, W, and L;
an IMGT LCDR1 that comprises the amino acid sequence of Q₂₇Z₂₈Z₂₉Z₃₀Z₃₁Z₃₂, wherein Z₂₈ is selected from the group consisting of: N, H, P, T, S, M, G, A, V, W, D, I, F, R, Y, Q, E, and L; Z₂₉ is selected from the group consisting of V, G, A, L, M, S, P, F, Q, T, D, K, W, Y, H, R and E; Z₃₀ is selected from the group consisting of: G, W, A, L, Q, N, R, M, P, D, K, H, I, V, S, E, and F; Z₃₁ is selected from the group consisting of I, E, Q, G, H, S, W, Y, M, N, P, F, A, V, K, D, and L; and Z₃₂ is selected from the group consisting of: N, S, H, A, K, Q, L, D, M, T, E, Y, V, F, W, R and P;
an IMGT LCDR2 that comprises the amino acid sequence of Z₅₀SZ₅₂, wherein Z₅₀ is selected from the group consisting of: S, V, A, G, H, L, T, M, F, Q, E, P, K, W, and Y; and Z₅₂ is selected from the group consisting of S, W, Y, G, T, M, F, N, L, A, V, Q, I, E, D, H, P, and K;
an IMGT LCDR3 that comprises the amino acid sequence of Z₈₉Z₉₀Z₉₁Z₉₂Z₉₃Z₉₄Z₉₅Z₉₆Z₉₇, wherein Z₈₉ is selected from the group consisting of: Q, Y, K, E, V, A, N, I, G, W, S, R, L, and H; Z₉₀ is selected from the group consisting of Q, M, H, E, N, L, D, G, V, R, I, Y, W, K, S, and P; Z₉₁ is selected from the group consisting of F, S, T, A, M, H, L, E, Y, W, N, R, Q, I, P, V, G, K, and D; Z₉₂ is selected from the group consisting of: K, G, I, R, S, V, Y, F, W, M, A, Q, T, H, E, L, D, N, and P; Z₉₃ is selected from the group consisting of S, F, R, A, L, M, K, D, W, N, V, Q, T, I, and G; Z₉₄ is selected from the group consisting of: Y, L, V, I, A, N, Q, R, E, G, P, S, K, D, W, and F; Z₉₅ is selected from the group consisting of P, N, A, G, S, H, V, D, K, T, L, E, M, Q, R, F, and W; Z₉₆ is selected from the group consisting of L, I, V, M, E, Y, F, N, A, G, H, R, S, W, Q, D, P, and T; and Z₉₇ is selected from the group consisting of: T, G, A, S, R, Q, V, W, E, L, I, H, M, F, N, D, and P; and wherein the polypeptide molecule does not comprise the amino acid sequence of SEQ ID NOs: 73, 113-116, 2458-2508, and SSS.

Embodiment 23. The polypeptide molecule of embodiment 22, wherein X₂₇ is Y; X₂₈ is selected from the group consisting of S, K, A, P, T, and V; X₂₉ is F; X₃₀ is selected from the group consisting of T, V, L, Y, W, and Q; X₃₁ is selected from the group consisting of T and L; or X₃₂ is selected from the group consisting of Y, F, M, W, L, R, and T.

Embodiment 24. The polypeptide molecule of embodiment 22 or 23, wherein X₂₈ is selected from the group consisting of K, A, P, and V; X₃₀ is selected from the group consisting of V, L, Y, W, and Q; or X₃₂ is selected from the group consisting of M, L, and R.

Embodiment 25. The polypeptide molecule of any one of embodiments 22-24, wherein X₃₁ is L.

Embodiment 26. The polypeptide molecule of any one of embodiments 22-25, wherein X₅₁ is F; X₅₂ is F; X₅₃ is P; X₅₄ is selected from the group consisting of G and R; X₅₅ is S; X₅₆ is G; X₅₇ is N; or X₅₈ is selected from the group consisting of I, V, Y, L, A, Q, K, T, and M.

Embodiment 27. The polypeptide molecule of any one of embodiments 22-26, wherein X₅₄ is R; or X₅₈ is selected from the group consisting of L, A, Q, K, and M.

Embodiment 28. The polypeptide molecule of any one of embodiments 22-27, wherein X₅₄ is R.

Embodiment 29. The polypeptide molecule of any one of embodiments 22-28, wherein X₉₈ is selected from the group consisting of G and A; X₉₉ is S; X₁₀₀ is Y; X₁₀₁ is Y; X₁₀₂ is S; X₁₀₃ is selected from the group consisting of Y and F; X₁₀₄ is D; X₁₀₅ is V; X₁₀₆ is L; X₁₀₇ is D; or X₁₀₈ is Y.

Embodiment 30. The polypeptide molecule of any one of embodiments 22-29, wherein X₉₈ is selected from the group consisting of G and A; or X₁₀₃ is F.

Embodiment 31. The polypeptide molecule of any one of embodiments 22-30, wherein X₉₈ is A; or X₁₀₃ is F.

Embodiment 32. The polypeptide molecule of any one of embodiments 22-31, wherein Z₂₈ is N; Z₂₉ is selected from the group consisting of V and G; Z₃₀ is G; Z₃₁ is I; or Z₃₂ is N.

Embodiment 33. The polypeptide molecule of any one of embodiments 22-32, wherein Z₂₉ is G; Z₃₁ is I; or Z₃₂ is N.

Embodiment 34. The polypeptide molecule of any one of embodiments 22-33, wherein Z₃₂ is R.

Embodiment 35. The polypeptide molecule of any one of embodiments 22-34, wherein Z₅₀ is S; or Z₅₂ is S.

Embodiment 36. The polypeptide molecule of any one of embodiments 22-35, wherein Z₈₉ is Q; Z₉₀ is Q; Z₉₁ is F; Z₉₂ is K; Z₉₃ is selected from the group consisting of S, F, R, A, and L; Z₉₄ is Y; Z₉₅ is selected from the group consisting of P and N; Z₉₆ is selected from the group consisting of L and I; or Z₉₇ is selected from the group consisting of T, G, and A.

Embodiment 37. The polypeptide molecule of any one of embodiments 22-36, wherein Z₉₃ is selected from the group consisting of F, R, A, and L; Z₉₅ is N; Z₉₆ is I; or Z₉₇ is selected from the group consisting of G and A.

Embodiment 38. The polypeptide molecule of any one of embodiments 22-37, wherein Z₉₆ is I.

Embodiment 39. A polypeptide molecule comprising an anti-TCRβV-binding domain, wherein the binding domain comprises:
an IMGT HCDR1 that comprises the amino acid sequence of G₂₆Y₂₇X₂₈F₂₉X₃₀X₃₁X₃₂Y₃₃, wherein X₂₈ is selected from the group consisting of S, K, A, P, T, and V; X₃₀ is selected from the group consisting of T, V, L, Y, W, and Q; X₃₁ is selected from the group consisting of: T and L; and X₃₂ is selected from the group consisting of Y, F, M, W, L, R, and T;
an IMGT HCDR2 that comprises the amino acid sequence of F₅₁F₅₂P₅₃X₅₄S₅₅G₅₆N₅₇X₅₈, wherein X₅₄ is selected from the group consisting of G and R; and X₅₈ is selected from the group consisting of: I, V, Y, L, A, Q, K, T, and M;
an IMGT HCDR3 that comprises the amino acid sequence of A₉₇X₉₈S₉₉Y₁₀₀Y₁₀₁S₁₀₂X₁₀₃D₁₀₄V₁₀₅L₁₀₆D₁₀₇Y₁₀₈, wherein X₉₈ is selected from the group consisting of: G and A; and wherein X₁₀₃ is selected from the group consisting of Y and F;
an IMGT LCDR1 that comprises the amino acid sequence of Q₂₇N₂₈Z₂₉G₃₀I₃₁Z₃₂, wherein Z₂₉ is selected from the group consisting of V and G; and wherein Z₃₂ is selected from the group consisting of N and R;
an IMGT LCDR2 that comprises the amino acid sequence of S₅₀S₅₁S₅₂;
an IMGT LCDR3 that comprises the amino acid sequence of Q₈₉Q₉₀F₉₁K₉₂Z₉₃Y₉₄Z₉₅Z₉₆Z₉₇, wherein Z₉₃ is selected from the group consisting of S, F, R, A, and L; Z₉₅ is selected from the group consisting of P and N; Z₉₆ is selected from the group consisting of L and I; and Z₉₇ is selected from the group consisting of: T, G, and A; and
wherein the polypeptide molecule does not comprise the amino acid sequence of SEQ ID NOs: 73, 113-116, and 2458-2508, and SSS.

Embodiment 40. A polypeptide molecule comprising an anti-TCRβV-binding domain, wherein the binding domain comprises:
an IMGT HCDR1 that comprises an amino acid sequence selected from any one of: GYSFTTYY (SEQ ID NO: 114), GYSFTLYY (SEQ ID NO: 139), GYSFTTWY (SEQ ID NO: 146), and GYSFTLWY (SEQ ID NO: 148);
an IMGT HCDR2 that comprises an amino acid sequence selected from any one of: FFPGSGNI (SEQ ID NO: 115) and FFPRSGNI (SEQ ID NO: 143)
an IMGT HCDR3 that comprises an amino acid sequence of AASYYSFDVLDY (SEQ ID NO: 140)
an IMGT LCDR1 that comprises an amino acid sequence selected from any one of: QNVGIN (SEQ ID NO: 113) and QNVGIR (SEQ ID NO: 138);
an IMGT LCDR2 that comprises an amino acid sequence of SSS;
an IMGT LCDR3 that comprises an amino acid sequence selected from any one of: QQFKSYPIT (SEQ ID NO: 135) and QQFKFYPIT (SEQ ID NO: 141);
wherein each of the HCDR1, HCDR2, HCDR3, LCDR1, LCDR2, and/or LCDR3 independently comprise 0, 1, 2, or 3 amino acid mutations.

Embodiment 41. A polypeptide molecule comprising an anti-TCRβV-binding domain, wherein the binding domain comprises:
an IMGT HCDR1 that comprises an amino acid sequence selected from any one of: GYSFTTYY (SEQ ID NO: 114), GYSFTLYY (SEQ ID NO: 139), GYSFTTWY (SEQ ID NO: 146), and GYSFTLWY (SEQ ID NO: 148);
an IMGT HCDR2 that comprises an amino acid sequence selected from any one of: FFPGSGNI (SEQ ID NO: 115) and FFPRSGNI (SEQ ID NO: 143)
an IMGT HCDR3 that comprises an amino acid sequence of AASYYSFDVLDY (SEQ ID NO: 140)
an IMGT LCDR1 that comprises an amino acid sequence selected from any one of: QNVGIN (SEQ ID NO: 113) and QNVGIR (SEQ ID NO: 138);
an IMGT LCDR2 that comprises an amino acid sequence of SSS;
an IMGT LCDR3 that comprises an amino acid sequence selected from any one of: QQFKSYPIT (SEQ ID NO: 135) and QQFKFYPIT (SEQ ID NO: 141).

Embodiment 42. A polypeptide molecule comprising an anti-TCRβV-binding domain, wherein the binding domain comprises:
(i) the HCDR1, the HCDR2, and the HCDR3 comprising the amino acid sequences of SEQ ID NOs: 139, 115, and 140, respectively, and the LCDR1, the LCDR2, and the LCDR3 comprising the amino acid sequences of SEQ ID NOs: 138, SSS, and 135, respectively;
(ii) the HCDR1, the HCDR2, and the HCDR3 comprising the amino acid sequences of SEQ ID NOs: 114, 143, and 140, respectively, and the LCDR1, the LCDR2, and the LCDR3 comprising the amino acid sequences of SEQ ID NOs: 138, SSS, and 141, respectively;
(iii) the HCDR1, the HCDR2, and the HCDR3 comprising the amino acid sequences of SEQ ID NOs: 114, 115, and 140, respectively, and the LCDR1, the LCDR2, and the LCDR3 comprising the amino acid sequences of SEQ ID NOs: 138, SSS, and 135, respectively;
(iv) the HCDR1, the HCDR2, and the HCDR3 comprising the amino acid sequences of SEQ ID NOs: 114, 143, and 140, respectively, and the LCDR1, the LCDR2, and the LCDR3 comprising the amino acid sequences of SEQ ID NOs: 138, SSS, and 135, respectively;
(v) the HCDR1, the HCDR2, and the HCDR3 comprising the amino acid sequences of SEQ ID NOs: 148, 115, and 140, respectively, and the LCDR1, the LCDR2, and the LCDR3 comprising the amino acid sequences of SEQ ID NOs: 138, SSS, and 135, respectively;
(vi) the HCDR1, the HCDR2, and the HCDR3 comprising the amino acid sequences of SEQ ID NOs: 148, 143, and 140, respectively, and the LCDR1, the LCDR2, and the LCDR3 comprising the amino acid sequences of SEQ ID NOs: 113, SSS, and 135, respectively;
(vii) the HCDR1, the HCDR2, and the HCDR3 comprising the amino acid sequences of SEQ ID NOs: 139, 143, and 140, respectively, and the LCDR1, the LCDR2, and the LCDR3 comprising the amino acid sequences of SEQ ID NOs: 138, SSS, and 135, respectively;
(viii) the HCDR1, the HCDR2, and the HCDR3 comprising the amino acid sequences of SEQ ID NOs: 114, 143, and 140, respectively, and the LCDR1, the LCDR2, and the LCDR3 comprising the amino acid sequences of SEQ ID NOs: 113, SSS, and 135, respectively;
(ix) the HCDR1, the HCDR2, and the HCDR3 comprising the amino acid sequences of SEQ ID NOs: 114, 143, and 140, respectively, and the LCDR1, the LCDR2, and the LCDR3 comprising the amino acid sequences of SEQ ID NOs: 138, SSS, and 135, respectively;
(x) the HCDR1, the HCDR2, and the HCDR3 comprising the amino acid sequences of SEQ ID NOs: 139, 143, and 140, respectively, and the LCDR1, the LCDR2, and the LCDR3 comprising the amino acid sequences of SEQ ID NOs: 138, SSS, and 135, respectively;
(xi) the HCDR1, the HCDR2, and the HCDR3 comprising the amino acid sequences of SEQ ID NOs: 146, 115, and 140, respectively, and the LCDR1, the LCDR2, and the LCDR3 comprising the amino acid sequences of SEQ ID NOs: 138, SSS, and 141, respectively;
(xii) the HCDR1, the HCDR2, and the HCDR3 comprising the amino acid sequences of SEQ ID NOs: 148, 115, and 140, respectively, and the LCDR1, the LCDR2, and the LCDR3 comprising the amino acid sequences of SEQ ID NOs: 138, SSS, and 141, respectively;
(xiii) the HCDR1, the HCDR2, and the HCDR3 comprising the amino acid sequences of SEQ ID NOs: 148, 115, and 140, respectively, and the LCDR1, the LCDR2, and the LCDR3 comprising the amino acid sequences of SEQ ID NOs: 138, SSS, and 135, respectively;
(xiv) the HCDR1, the HCDR2, and the HCDR3 comprising the amino acid sequences of SEQ ID NOs: 114, 143, and 140, respectively, and the LCDR1, the LCDR2, and the LCDR3 comprising the amino acid sequences of SEQ ID NOs: 138, SSS, and 141, respectively;
(xv) the HCDR1, the HCDR2, and the HCDR3 comprising the amino acid sequences of SEQ ID NOs: 139, 115, and 140, respectively, and the LCDR1, the LCDR2, and the LCDR3 comprising the amino acid sequences of SEQ ID NOs: 138, SSS, and 141, respectively;
(xvi) the HCDR1, the HCDR2, and the HCDR3 comprising the amino acid sequences of SEQ ID NOs: 148, 143, and 140, respectively, and the LCDR1, the LCDR2, and the LCDR3 comprising the amino acid sequences of SEQ ID NOs: 138, SSS, and 135, respectively;
(xvii) the HCDR1, the HCDR2, and the HCDR3 comprising the amino acid sequences of SEQ ID NOs: 139, 143, and 140, respectively, and the LCDR1, the LCDR2, and the LCDR3 comprising the amino acid sequences of SEQ ID NOs: 138, SSS, and 135, respectively; or
(xviii) the HCDR1, the HCDR2, and the HCDR3 comprising the amino acid sequences of SEQ ID NOs: 148, 143, and 140, respectively, and the LCDR1, the LCDR2, and the LCDR3 comprising the amino acid sequences of SEQ ID NOs: 138, SSS, and 141, respectively.

Embodiment 43. The polypeptide molecule of any one of embodiments 1-42, wherein the polypeptide molecule comprises an immunoglobulin heavy chain variable (VH) region and/or an immunoglobulin light chain variable (VL) region.

Embodiment 44. The polypeptide molecule of embodiment 43, wherein the VH region comprises an amino acid sequence that is at least about 80%, at least about 85%, at least about 90%, at least about 95%, at least about 96%, at least about 97%, at least about 98%, at least about 99%, or 100% identical to any one of SEQ ID NOs: 100-111, 2512, and 2513.

Embodiment 45. The polypeptide molecule of embodiment 43, wherein the VL region comprises an amino acid sequence that is at least about 80%, at least about 85%, at least about 90%, at least about 95%, at least about 96%, at least about 97%, at least about 98%, at least about 99%, or 100% identical to any one of SEQ ID NOs: 85-93 and 2514.

Embodiment 46. The polypeptide molecule of embodiment 43, wherein the VH region comprises an amino acid sequence that is at least about 80%, at least about 85%, at least about 90%, at least about 95%, at least about 96%, at least about 97%, at least about 98%, at least about 99%, or 100% identical to any one of SEQ ID NOs: 100-111, 2512, and 2513; and the VL region comprises an amino acid sequence that is at least about 80%, at least about 85%, at least about 90%, at least about 95%, at least about 96%, at least about 97%, at least about 98%, at least about 99%, or 100% identical to any one of SEQ ID NOs: 85-93 and 2514.

Embodiment 47. The polypeptide molecule of any one of embodiments 1-46, wherein the TCRβV-binding domain is an scFv, and the scFv comprises an amino acid sequence that is at least about 80%, at least about 85%, at least about 90%, at least about 95%, at least about 96%, at least about 97%, at least about 98%, at least about 99%, or 100% identical to any one of SEQ ID NOs: 149-222.

Embodiment 48. The polypeptide molecule of any one of embodiments 1-46, wherein the TCRβV-binding domain comprises a Fab or a CrossFab, and the Fab or CrossFab comprises (i) a light chain sequence that is at least about 80%, at least about 85%, at least about 90%, at least about 95%, at least about 96%, at least about 97%, at least about 98%, at least about 99%, or 100% identical to any one of SEQ ID NOs: 230-574; and (ii) a heavy chain sequence that is at least about 80%, at least about 85%, at least about 90%, at least about 95%, at least about 96%, at least about 97%, at least about 98%, at least about 99%, or 100% identical to any one of SEQ ID NOs: 575-1084.

Embodiment 49. A polypeptide molecule comprising an anti-TCRβV binding domain, wherein the binding domain comprises:
a Kabat heavy chain CDR1 (HCDR1) that comprises the amino acid sequence of X₃₁X₃₂G₃₃M₃₄X₃₅, wherein X₃₁ is selected from the group consisting of: N, R, I, Y, V, H, L, T, S, Q, D, F, K, M, A, W, G, E, and P; X₃₂ is selected from the group consisting of F, K, T, Y, V, R, A, H, I, G, M, L, Q, N, E, W, D, and P; and X₃₅ is selected from the group consisting of H, W, F, N, G, S, D, M, A, Y, L, T, I, E, Q, K, and P;
a Kabat heavy chain CDR2 (HCDR2) that comprises the amino acid sequence of Y₅₀I₅₁S₅₂X₅₃X₅₄X₅₅X₅₆X₅₇X₅₈X₅₉Y₆₀X₆₁X₆₂X₆₃L₆₄K₆₅G₆₆, wherein X₅₃ is selected from the group consisting of: S, T, G, K, A, L, W, Y, E, D, I, M, V, N, P, F, H, and Q; X₅₄ is selected from the group consisting of G, W, S, Y, T, Q, V, R, H, E, M, K, P, N, F, A, L, I, and D; X₅₅ is selected from the group consisting of S, H, G, Q, A, T, L, M, E, W, N, K, R, Y, D, P, I, and F; X₅₆ is selected from the group consisting of: S, M, T, P, K, R, Q, Y, L, G, N, H, I, D, V, F, W, and E; X₅₇ is selected from the group consisting of T, V, S, L, A, I, P, M, R, K, Q, H, W, F, Y, E, and D; X₅₈ is selected from the group consisting of: I, F, W, Y, V, E, L, M, A, K, R, T, D, N, S, P, H, and G; X₅₉ is selected from the group consisting of: Y, L, I, H, M, F, R, K, V, T, Q, G, P, A, S, W, and D; X₆₁ is selected from the group consisting of A, K, R, D, Q, W, N, S, Y, G, F, E, P, H, T, V, I, L, and M; X₆₂ is selected from the group consisting of D, P, T, E, H, G, R, N, Q, K, I, Y, W, L, F, and V; and X₆₃ is selected from the group consisting of T, I, K, N, H, E, G, M, R, Y, V, F, L, P, S, Q, A, W, and D;
a Kabat heavy chain CDR3 (HCDR3) that comprises the amino acid sequence of X₉₉X₁₀₀X₁₀₁X₁₀₂X₁₀₃X₁₀₄X₁₀₅X₁₀₆, wherein X₉₉ is selected from the group consisting of R, I, K, L, P, N, S, Y, V, A, G, T, D, Q, H, M, and W; X₁₀₀ is selected from the group consisting of: G, N, H, S, K, Y, M, R, F, L, D, E, T, I, W, V, and P; X₁₀₁ is selected from the group consisting of: E, H, L, A, T, M, R, S, G, N, Y, W, K, I, P, V, and D; X₁₀₂ is selected from the group consisting of G, W, H, Y, K, R, A, F, N, V, M, S, T, E, L, P, I, and D; X₁₀₃ is selected from the group consisting of: A, S, K, Y, G, T, V, P, R, F, W, N, I, Q, L, H, M, D, and E; X₁₀₄ is selected from the group consisting of M, L, A, P, F, G, E, N, T, S, R, H, Q, W, Y, and D; X₁₀₅ is selected from the group consisting of: D, I, H, Q, Y, K, T, W, L, R, S, E, M, G, P, F, and V; and X₁₀₆ is selected from the group consisting of: Y, N, A, W, V, M, P, T, Q, R, S, H, L, F, G, and D;
a Kabat light chain CDR1 (LCDR1) that comprises the amino acid sequence of R₂₄A₂₅Z₂₆Z₂₇Z₂₈Z₂₉Z₃₀Z₃₁Z₃₂Z₃₃, wherein Z₂₆ is selected from the group consisting of: S, M, V, L, Q, R, H, Y, I, T, F, A, W, N, P, G, D, and K; Z₂₇ is selected from the group consisting of S, K, R, H, Y, N, I, Q, T, G, M, L, F, V, A, W, P, E, and D; Z₂₈ is selected from the group consisting of: S, K, N, R, P, M, Y, A, I, H, V, Q, W, D, F, E, T, and G; Z₂₉ is selected from the group consisting of I, T, L, A, R, M, G, P, Q, Y, F, H, W, N, E, and D; Z₃₀ is selected from the group consisting of: N, V, R, K, M, A, T, L, W, E, Y, Q, S, H, G, P, F, and D; Z₃₁ is selected from the group consisting of Y, F, Y, K, R, H, A, N, M, Q, V, P, G, L, T, E, I, and D; Z₃₂ is selected from the group consisting of: I, A, S, M, L, T, Q, D, P, H, F, N, K, W, Y, and R; and Z₃₃ is selected from the group consisting of Y, D, W, S, L, F, H, T, R, G, P, A, N, V, and I;
a Kabat light chain CDR2 (LCDR2) that comprises the amino acid sequence of Z₄₉Z₅₀Z₅₁Z₅₂Z₅₃Z₅₄Z₅₅, wherein Z₄₉ is selected from the group consisting of: Y, K, Y, F, I, T, H, M, W, L, Q, A, G, P, V, N, E, and D; Z₅₀ is selected from the group consisting of: T, P, S, I, Q, A, W, F, M, N, G, L, Y, R, K, D, E, and H; Z₅₁ is selected from the group consisting of: S, N, K, R, P, D, L, T, V, Y, H, A, Q, W, I, F, C, E, and G; Z₅₂ is selected from the group consisting of N, R, K, A, L, V, F, M, W, G, E, Y, H, D, I, S, and P; Z₅₃ is selected from the group consisting of L, H, K, A, N, Y, R, F, P, V, C, I, W, G, E, D, and T; Z₅₄ is selected from the group consisting of: A, R, V, I, H, F, K, T, P, Q, N, G, E, D, and Y; and Z₅₅ is selected from the group consisting of P, R, T, Q, V, L, W, E, D, G, I, H, S, M, F, N, and Y;
a Kabat light chain CDR3 (LCDR3) that comprises the amino acid sequence of Z₈₈Z₈₉Z₉₀Z₉₁Z₉₂Z₉₃Z₉₄Z₉₅Z₉₆, wherein Z₈₈ is selected from the group consisting of: Q, E, L, Y, N, M, P, D, S, V, H, T, W, I, F, and R; Z₈₉ is selected from the group consisting of: Q, H, E, K, V, R, T, G, S, A, L, I, F, M, Y, D, and P; Z₉₀ is selected from the group consisting of F, S, L, K, M, Y V, Q, N, A, R, I, T, H, G, W, D, P, and E; Z₉₁ is selected from the group consisting of T, S, I, H, N, A, K, G, R, Q, Y, M, F, L, V, P, W, E, and D; Z₉₂ is selected from the group consisting of: S, G, R, A, N, Y, M, W, D, T, F, V, K, Q, H, L, I, E, and P; Z₉₃ is selected from the group consisting of S, K, A, F, R, P, H, T, V, M, L, E, N, Q, W, D, and G; Z₉₄ is selected from the group consisting of: P, W, E, I, Y, T, F, H, L, A, S, C, R, K, N, Q, and V; Z₉₅ is selected from the group consisting of F, L, P, M, V, W, A, N, S, I, G, E, H, Q, and R; and Z₉₆ is selected from the group consisting of T, V, S, H, Y, L, F, I, W, R, Q, M, E, K, G, and D;
wherein the polypeptide molecule does not comprise SEQ ID NOs: 1148, 1156, 1157, 1160, 1171, and 1180.

Embodiment 50. The polypeptide molecule of embodiment 49, wherein X₃₁ is selected from the group consisting of N, R, I, Y, V, H, L, T, S, Q, and D, X₃₂ is selected from the group consisting of F, K, T, Y, V, R, A, H, and I; or X₃₅ is H.

Embodiment 51. The polypeptide molecule of embodiment 49 or 50, wherein X₃₁ is selected from the group consisting of R and I; or X₃₂ is T.

Embodiment 52. The polypeptide molecule of any one of embodiments 49-51, wherein X₅₃ is S; X₅₄ is selected from the group consisting of G, W, S, Y, T, Q, V, R, H, E, M, K, and P; X₅₅ is selected from the group consisting of S, H; X₅₆ is S; X₅₇ is selected from the group consisting of T and V; X₅₈ is selected from the group consisting of I, F, W, and Y; X₅₉ is selected from the group consisting of Y, L, I, and H; X₆₁ is selected from the group consisting of A, K, R, D, Q, W, N, S, Y, G, and F; X₆₂ is selected from the group consisting of D, P, and T, or X₆₃ is selected from the group consisting of T, I, K, N, H, E, G, M, R, Y, V, F, L, and P.

Embodiment 53. The polypeptide molecule of any one of embodiments 49-52, wherein X₅₄ is selected from the group consisting of S and T; or X₅₉ is L.

Embodiment 54. The polypeptide molecule of any one of embodiments 49-53, wherein X₉₉ is R; X₁₀₀ is G; X₁₀₁ is selected from the group consisting of E, H, L, A, T, M, R, S, G, N, Y, W, K, I, and P; X₁₀₂ is G; X₁₀₃ is A; X₁₀₄ is M; X₁₀₅ is D; or X₁₀₆ is selected from the group consisting of Y, N, A, W, V, M, P, and T.

Embodiment 55. The polypeptide molecule of any one of embodiments 49-54, wherein X₁₀₁ is A.

Embodiment 56. The polypeptide molecule of any one of embodiments 49-55, wherein Z₂₆ is selected from the group consisting of S, M, V, and L; Z₂₇ is selected from the group consisting of S, K, R, and H; Z₂₈ is selected from the group consisting of S, K, N, R, and P; Z₂₉ is I; Z₃₀ is selected from the group consisting of N and V; Z₃₁ is selected from the group consisting of Y and F; Z₃₂ is selected from the group consisting of I, A, and S; or Z₃₃ is Y.

Embodiment 57. The polypeptide molecule of any one of embodiments 49-56, wherein Z₂₇ is selected from the group consisting of K and R; or Z₃₂ is A.

Embodiment 58. The polypeptide molecule of any one of embodiments 49-57, wherein Z₄₉ is selected from the group consisting of Y and K; Z₅₀ is selected from the group consisting of T and P; Z₅₁ is selected from the group consisting of S, N, K, R, P, D, and L; Z₅₂ is selected from the group consisting of N, R, K, A, and L; Z₅₃ is selected from the group consisting of L and H; Z₅₄ is selected from the group consisting of A, R, and V; or Z₅₅ is selected from the group consisting of P, R, T, Q, V, and L.

Embodiment 59. The polypeptide molecule of any one of embodiments 49-58, wherein Z₈₈ is Q; Z₈₉ is Q; Z₉₀ is F; Z₉₁ is T; Z₉₂ is selected from the group consisting of S, G, and R; Z₉₃ is selected from the group consisting of S, K, A, F, R, P, and H; Z₉₄ is P; Z₉₅ is F; or Z₉₆ is selected from the group consisting of T, V, S, H, and Y.

Embodiment 60. The polypeptide molecule of any one of embodiments 49-59, wherein Z₉₆ is V.

Embodiment 61. A polypeptide molecule comprising an anti-TCRβV, wherein the binding molecule comprises:
a Kabat HCDR1 that comprises the amino acid sequence of X₃₁X₃₂G₃₃M₃₄H₃₅, wherein X₃₁ is selected from the group consisting of: N, R, I, Y, V, H, L, T, S, Q, and D; and X₃₂ is selected from the group consisting of F, K, T, Y, V, R, A, H, and I;
a Kabat HCDR2 that comprises the amino acid sequence of Y₅₀I₅₁S₅₂S₅₃X₅₄X₅₅S₅₆X₅₇X₅₈X₅₉Y₆₀X₆₁X₆₂X₆₃L₆₄K₆₅G₆₆, wherein X₅₄ is selected from the group consisting of G, W, S, Y, T, Q, V, R, H, E, M, K, and P; X₅₅ is selected from the group consisting of S and H; X₅₇ is selected from the group consisting of T and V; X₅₈ is selected from the group consisting of: I, F, W, and Y; X₅₉ is selected from the group consisting of: Y, L, I, and H; X₆₁ is selected from the group consisting of A, K, R, D, Q, W, N, S, Y, G, and F; X₆₂ is selected from the group consisting of D, P, and T; and X₆₃ is selected from the group consisting of T, I, K, N, H, E, G, M, R, Y, V, F, L, and P;
a Kabat HCDR3 that comprises the amino acid sequence of R₉₉G₁₀₀X₁₀₁G₁₀₂A₁₀₃M₁₀₄D₁₀₅X₁₀₆, wherein X₁₀₁ is selected from the group consisting of: E, H, L, A, T, M, R, S, G, N, Y, W, K, I, and P; and X₁₀₆ is selected from the group consisting of: Y, N, A, W, V, M, P, and T;
a Kabat LCDR1 that comprises the amino acid sequence of R₂₄A₂₅Z₂₆Z₂₇Z₂₈I₂₉Z₃₀Z₃₁Z₃₂Y₃₃, wherein Z₂₆ is selected from the group consisting of: S, M, V, and L; Z₂₇ is selected from the group consisting of S, K, R, and H; Z₂₈ is selected from the group consisting of: S, K, N, R, and P; Z₃₀ is selected from the group consisting of: N and V; Z₃₁ is selected from the group consisting of Y and F; Z₃₂ is selected from the group consisting of: I, A, and S;
a Kabat LCDR2 that comprises the amino acid sequence of Z₄₉Z₅₀Z₅₁Z₅₂Z₅₃Z₅₄Z₅₅, wherein Z₄₉ is selected from the group consisting of: Y and K; Z₅₀ is selected from the group consisting of: T and P; Z₅₁ is selected from the group consisting of: S, N, K, R, P, D, and L; Z₅₂ is selected from the group consisting of N, R, K, A, and L; Z₅₃ is selected from the group consisting of L and H; Z₅₄ is selected from the group consisting of: A, R, and V; and Z₅₅ is selected from the group consisting of P, R, T, Q, V, and L;
a Kabat LCDR3 that comprises the amino acid sequence of Q₈₈Q₈₉F₉₀T₉₁Z₉₂Z₉₃P₉₄F₉₅Z₉₆, wherein Z₉₂ is selected from the group consisting of: S, G, and R; Z₉₃ is selected from the group consisting of S, K, A, F, R, P, and H; and Z₉₆ is selected from the group consisting of T, V, S, H, and Y.
wherein the polypeptide molecule does not comprise SEQ ID NOs: 1148, 1156, 1157, 1160, 1171, and 1180.

Embodiment 62. A polypeptide molecule comprising an anti-TCRβV-binding domain, wherein the binding domain comprises:
a Kabat HCDR1 that comprises an amino acid sequence selected from any one of: NFGMH (SEQ ID NO: 1160); IFGMH (SEQ ID NO: 1162); RTGMH (SEQ ID NO: 1163); IKGMH (SEQ ID NO: 1164); NKGMH (SEQ ID NO: 1165); RFGMH (SEQ ID NO: 1166); RKGMH (SEQ ID NO: 1167); ITGMH (SEQ ID NO: 1168); IYGMH (SEQ ID NO: 1169); and NTGMH (SEQ ID NO: 1170);
a Kabat HCDR2 that comprises an amino acid sequence selected from any one of: YISSGSSTIYYADTLKG (SEQ ID NO: 1171), YISSTSSTILYADTLKG (SEQ ID NO: 1173), YISSSSSTILYADTLKG (SEQ ID NO: 1174), YISSSSSTIYYADTLKG (SEQ ID NO: 1175), YISSWSSTILYADTLKG (SEQ ID NO: 1176), YISSWSSTIYYADTLKG (SEQ ID NO: 1177), YISSTSSTIYYADTLKG (SEQ ID NO: 1178), and YISSGSSTILYADTLKG (SEQ ID NO: 1179);
a Kabat HCDR3 that comprises an amino acid sequence of RGEGAMDY (SEQ ID NO: 1180) and RGAGAMDY (SEQ ID NO: 1181) a Kabat LCDR1 that comprises an amino acid sequence selected from any one of: RASSSVNYIY (SEQ ID NO: 1148), RASRSVNYAY (SEQ ID NO: 1151), RASKSVNYAY (SEQ ID NO: 1152), RASKSVNYIY (SEQ ID NO: 1153), RASSSVNYAY (SEQ ID NO: 1154), and RASRSVNYIY (SEQ ID NO: 1155);
a Kabat LCDR2 that comprises an amino acid sequence of YTSNLAP (SEQ ID NO: 1156);
a Kabat LCDR3 that comprises an amino acid sequence selected from any one of: QQFTSSPFT (SEQ ID NO: 1157) and QQFTSSPFV (SEQ ID NO: 1159);
wherein each of the HCDR1, HCDR2, HCDR3, LCDR1, LCDR2, and/or LCDR3 independently comprise 0, 1, 2, or 3 amino acid mutations.

Embodiment 63. A polypeptide molecule comprising an anti-TCRβV-binding domain, wherein the binding domain comprises:
a Kabat HCDR1 that comprises an amino acid sequence selected from any one of: NFGMH (SEQ ID NO: 1160); IFGMH (SEQ ID NO: 1162); RTGMH (SEQ ID NO: 1163); IKGMH (SEQ ID NO: 1164); NKGMH (SEQ ID NO: 1165); RFGMH (SEQ ID NO: 1166); RKGMH (SEQ ID NO: 1167); ITGMH (SEQ ID NO: 1168); IYGMH (SEQ ID NO: 1169); and NTGMH (SEQ ID NO: 1170);
a Kabat HCDR2 that comprises an amino acid sequence selected from any one of: YISSGSSTIYYADTLKG (SEQ ID NO: 1171), YISSTSSTILYADTLKG (SEQ ID NO: 1173), YISSSSSTILYADTLKG (SEQ ID NO: 1174), YISSSSSTIYYADTLKG (SEQ ID NO: 1175), YISSWSSTILYADTLKG (SEQ ID NO: 1176), YISSWSSTIYYADTLKG (SEQ ID NO: 1177), YISSTSSTIYYADTLKG (SEQ ID NO: 1178), and YISSGSSTILYADTLKG (SEQ ID NO: 1179);
a Kabat HCDR3 that comprises an amino acid sequence of RGEGAMDY (SEQ ID NO: 1180) and RGAGAMDY (SEQ ID NO: 1181) a Kabat LCDR1 that comprises an amino acid sequence selected from any one of: RASSSVNYIY (SEQ ID NO: 1148), RASRSVNYAY (SEQ ID NO: 1151), RASKSVNYAY (SEQ ID NO: 1152), RASKSVNYIY (SEQ ID NO: 1153), RASSSVNYAY (SEQ ID NO: 1154), and RASRSVNYIY (SEQ ID NO: 1155);
a Kabat LCDR2 that comprises an amino acid sequence of YTSNLAP (SEQ ID NO: 1156);
a Kabat LCDR3 that comprises an amino acid sequence selected from any one of: QQFTSSPFT (SEQ ID NO: 1157) and QQFTSSPFV (SEQ ID NO: 1159).

Embodiment 64. The polypeptide of embodiment 65, wherein the binding domain comprises:
(i) the HCDR1, the HCDR2, and the HCDR3 comprising the amino acid sequences of SEQ ID NOs: 1162, 1173, and 1180, respectively, and the LCDR1, the LCDR2, and the LCDR3 comprising the amino acid sequences of SEQ ID NOs: 1151, 1156, and 1159, respectively;
(ii) the HCDR1, the HCDR2, and the HCDR3 comprising the amino acid sequences of SEQ ID NOs: 1162, 1173, and 1180, respectively, and the LCDR1, the LCDR2, and the LCDR3 comprising the amino acid sequences of SEQ ID NOs: 1148, 1156, and 1159, respectively;
(iii) the HCDR1, the HCDR2, and the HCDR3 comprising the amino acid sequences of SEQ ID NOs: 1163, 1174, and 1180, respectively, and the LCDR1, the LCDR2, and the LCDR3 comprising the amino acid sequences of SEQ ID NOs: 1151, 1156, and 1159, respectively;
(iv) the HCDR1, the HCDR2, and the HCDR3 comprising the amino acid sequences of SEQ ID NOs: 1164, 1171, and 1181, respectively, and the LCDR1, the LCDR2, and the LCDR3 comprising the amino acid sequences of SEQ ID NOs: 1152, 1156, and 1159, respectively;
(v) the HCDR1, the HCDR2, and the HCDR3 comprising the amino acid sequences of SEQ ID NOs: 1165, 1175, and 1180, respectively, and the LCDR1, the LCDR2, and the LCDR3 comprising the amino acid sequences of SEQ ID NOs: 1153, 1156, and 1157, respectively;
(vi) the HCDR1, the HCDR2, and the HCDR3 comprising the amino acid sequences of SEQ ID NOs: 1160, 1176, and 1181, respectively, and the LCDR1, the LCDR2, and the LCDR3 comprising the amino acid sequences of SEQ ID NOs: 1154, 1156, and 1159, respectively;
(vii) the HCDR1, the HCDR2, and the HCDR3 comprising the amino acid sequences of SEQ ID NOs: 1162, 1177, and 1180, respectively, and the LCDR1, the LCDR2, and the LCDR3 comprising the amino acid sequences of SEQ ID NOs: 1151, 1156, and 1157, respectively;
(viii) the HCDR1, the HCDR2, and the HCDR3 comprising the amino acid sequences of SEQ ID NOs: 1166, 1176, and 1180, respectively, and the LCDR1, the LCDR2, and the LCDR3 comprising the amino acid sequences of SEQ ID NOs: 1153, 1156, and 1159, respectively;
(ix) the HCDR1, the HCDR2, and the HCDR3 comprising the amino acid sequences of SEQ ID NOs: 1167, 1173, and 1180, respectively, and the LCDR1, the LCDR2, and the LCDR3 comprising the amino acid sequences of SEQ ID NOs: 1155, 1156, and 1159, respectively;
(x) the HCDR1, the HCDR2, and the HCDR3 comprising the amino acid sequences of SEQ ID NOs: 1162, 1177, and 1181, respectively, and the LCDR1, the LCDR2, and the LCDR3 comprising the amino acid sequences of SEQ ID NOs: 1154, 1156, and 1159, respectively;
(xi) the HCDR1, the HCDR2, and the HCDR3 comprising the amino acid sequences of SEQ ID NOs: 1162, 1174, and 1181, respectively, and the LCDR1, the LCDR2, and the LCDR3 comprising the amino acid sequences of SEQ ID NOs: 1154, 1156, and 1159, respectively;
(xii) the HCDR1, the HCDR2, and the HCDR3 comprising the amino acid sequences of SEQ ID NOs: 1165, 1174, and 1181, respectively, and the LCDR1, the LCDR2, and the LCDR3 comprising the amino acid sequences of SEQ ID NOs: 1154, 1156, and 1157, respectively;
(xiii) the HCDR1, the HCDR2, and the HCDR3 comprising the amino acid sequences of SEQ ID NOs: 1166, 1178, and 1180, respectively, and the LCDR1, the LCDR2, and the LCDR3 comprising the amino acid sequences of SEQ ID NOs: 1151, 1156, and 1157, respectively;
(xiv) the HCDR1, the HCDR2, and the HCDR3 comprising the amino acid sequences of SEQ ID NOs: 1168, 1173, and 1181, respectively, and the LCDR1, the LCDR2, and the LCDR3 comprising the amino acid sequences of SEQ ID NOs: 1153, 1156, and 1159, respectively;
(xv) the HCDR1, the HCDR2, and the HCDR3 comprising the amino acid sequences of SEQ ID NOs: 1167, 1171, and 1181, respectively, and the LCDR1, the LCDR2, and the LCDR3 comprising the amino acid sequences of SEQ ID NOs: 1154, 1156, and 1157, respectively;
(xvi) the HCDR1, the HCDR2, and the HCDR3 comprising the amino acid sequences of SEQ ID NOs: 1168, 1177, and 1181, respectively, and the LCDR1, the LCDR2, and the LCDR3 comprising the amino acid sequences of SEQ ID NOs: 1153, 1156, and 1157, respectively;
(xvii) the HCDR1, the HCDR2, and the HCDR3 comprising the amino acid sequences of SEQ ID NOs: 1164, 1174, and 1181, respectively, and the LCDR1, the LCDR2, and the LCDR3 comprising the amino acid sequences of SEQ ID NOs: 1153, 1156, and 1159, respectively;
(xviii) the HCDR1, the HCDR2, and the HCDR3 comprising the amino acid sequences of SEQ ID NOs: 1167, 1177, and 1181, respectively, and the LCDR1, the LCDR2, and the LCDR3 comprising the amino acid sequences of SEQ ID NOs: 1154, 1156, and 1157, respectively;
(xix) the HCDR1, the HCDR2, and the HCDR3 comprising the amino acid sequences of SEQ ID NOs: 1162, 1173, and 1180, respectively, and the LCDR1, the LCDR2, and the LCDR3 comprising the amino acid sequences of SEQ ID NOs: 1151, 1156, and 1157, respectively;
(xx) the HCDR1, the HCDR2, and the HCDR3 comprising the amino acid sequences of SEQ ID NOs: 1169, 1173, and 1181, respectively, and the LCDR1, the LCDR2, and the LCDR3 comprising the amino acid sequences of SEQ ID NOs: 1154, 1156, and 1157, respectively;
(xxi) the HCDR1, the HCDR2, and the HCDR3 comprising the amino acid sequences of SEQ ID NOs: 1163, 1179, and 1180, respectively, and the LCDR1, the LCDR2, and the LCDR3 comprising the amino acid sequences of SEQ ID NOs: 1152, 1156, and 1159, respectively;
(xxii) the HCDR1, the HCDR2, and the HCDR3 comprising the amino acid sequences of SEQ ID NOs: 1170, 1173, and 1181, respectively, and the LCDR1, the LCDR2, and the LCDR3 comprising the amino acid sequences of SEQ ID NOs: 1151, 1156, and 1157, respectively;
(xxiii) the HCDR1, the HCDR2, and the HCDR3 comprising the amino acid sequences of SEQ ID NOs: 1165, 1179, and 1180, respectively, and the LCDR1, the LCDR2, and the LCDR3 comprising the amino acid sequences of SEQ ID NOs: 1152, 1156, and 1157, respectively;
(xxiv) the HCDR1, the HCDR2, and the HCDR3 comprising the amino acid sequences of SEQ ID NOs: 1168, 1173, and 1181, respectively, and the LCDR1, the LCDR2, and the LCDR3 comprising the amino acid sequences of SEQ ID NOs: 1155, 1156, and 1159, respectively;
(xxv) the HCDR1, the HCDR2, and the HCDR3 comprising the amino acid sequences of SEQ ID NOs: 1166, 1178, and 1180, respectively, and the LCDR1, the LCDR2, and the LCDR3 comprising the amino acid sequences of SEQ ID NOs: 1151, 1156, and 1159, respectively;
(xxvi) the HCDR1, the HCDR2, and the HCDR3 comprising the amino acid sequences of SEQ ID NOs: 1168, 1175, and 1181, respectively, and the LCDR1, the LCDR2, and the LCDR3 comprising the amino acid sequences of SEQ ID NOs: 1152, 1156, and 1159, respectively;
(xxvii) the HCDR1, the HCDR2, and the HCDR3 comprising the amino acid sequences of SEQ ID NOs: 1163, 1177, and 1180, respectively, and the LCDR1, the LCDR2, and the LCDR3 comprising the amino acid sequences of SEQ ID NOs: 1154, 1156, and 1159, respectively;
(xxviii) the HCDR1, the HCDR2, and the HCDR3 comprising the amino acid sequences of SEQ ID NOs: 1167, 1177, and 1181, respectively, and the LCDR1, the LCDR2, and the LCDR3 comprising the amino acid sequences of SEQ ID NOs: 1153, 1156, and 1159, respectively;
(xxix) the HCDR1, the HCDR2, and the HCDR3 comprising the amino acid sequences of SEQ ID NOs: 1160, 1177, and 1181, respectively, and the LCDR1, the LCDR2, and the LCDR3 comprising the amino acid sequences of SEQ ID NOs: 1153, 1156, and 1159, respectively;
(xxx) the HCDR1, the HCDR2, and the HCDR3 comprising the amino acid sequences of SEQ ID NOs: 1170, 1173, and 1180, respectively, and the LCDR1, the LCDR2, and the LCDR3 comprising the amino acid sequences of SEQ ID NOs: 1151, 1156, and 1159, respectively;
(xxxi) the HCDR1, the HCDR2, and the HCDR3 comprising the amino acid sequences of SEQ ID NOs: 1170, 1174, and 1181, respectively, and the LCDR1, the LCDR2, and the LCDR3 comprising the amino acid sequences of SEQ ID NOs: 1151, 1156, and 1159, respectively;
(xxxii) the HCDR1, the HCDR2, and the HCDR3 comprising the amino acid sequences of SEQ ID NOs: 1163, 1176, and 1181, respectively, and the LCDR1, the LCDR2, and the LCDR3 comprising the amino acid sequences of SEQ ID NOs: 1151, 1156, and 1159, respectively;
(xxxiii) the HCDR1, the HCDR2, and the HCDR3 comprising the amino acid sequences of SEQ ID NOs: 1165, 1176, and 1181, respectively, and the LCDR1, the LCDR2, and the LCDR3 comprising the amino acid sequences of SEQ ID NOs: 1154, 1156, and 1157, respectively;
(xxxiv) the HCDR1, the HCDR2, and the HCDR3 comprising the amino acid sequences of SEQ ID NOs: 1165, 1179, and 1181, respectively, and the LCDR1, the LCDR2, and the LCDR3 comprising the amino acid sequences of SEQ ID NOs: 1151, 1156, and 1159, respectively;
(xxxv) the HCDR1, the HCDR2, and the HCDR3 comprising the amino acid sequences of SEQ ID NOs: 1163, 1174, and 1180, respectively, and the LCDR1, the LCDR2, and the LCDR3 comprising the amino acid sequences of SEQ ID NOs: 1152, 1156, and 1159, respectively;
(xxxvi) the HCDR1, the HCDR2, and the HCDR3 comprising the amino acid sequences of SEQ ID NOs: 1170, 1175, and 1181, respectively, and the LCDR1, the LCDR2, and the LCDR3 comprising the amino acid sequences of SEQ ID NOs: 1151, 1156, and 1159, respectively; or
(xxxvii) the HCDR1, the HCDR2, and the HCDR3 comprising the amino acid sequences of SEQ ID NOs: 1168, 1174, and 1180, respectively, and the LCDR1, the LCDR2, and the LCDR3 comprising the amino acid sequences of SEQ ID NOs: 1155, 1156, and 1159, respectively.

Embodiment 65. A polypeptide molecule comprising an anti-TCRβV binding domain, wherein the binding domain comprises:
an IMGT HCDR1 that comprises the amino acid sequence of G₂₆X₂₇X₂₈X₂₉X₃₀X₃₁X₃₂G₃₃, wherein X₂₇ is selected from the group consisting of: F, Y, W, M, I, L, H, V, A, Q, G, T, N, P, S, R, K, and E; X₂₈ is selected from the group consisting of T, K, S, M, N, I, F, H, G, Q, R, W, V, Y, E, A, D, and L; X₂₉ is selected from the group consisting of: F, Y, W, M, I, T, A, E, L, V, S, P, N, Q, H, G, K, R, and D; X₃₀ is selected from the group consisting of S, H, R, Q, W, F, K, N, Y, M, V, G, P, L, E, D, and I; X₃₁ is selected from the group consisting of: N, R, I, Y, V, H, L, T, S, Q, D, F, K, M, A, W, G, E, and P; and X₃₂ is selected from the group consisting of F, K, T, Y V, R, A, H, I, G, M, L, Q, N, E, W, D, and P;
an IMGT HCDR2 that comprises the amino acid sequence of I₅₁S₅₂X₅₃X₅₄X₅₅X₅₆X₅₇X₅₈, wherein X₅₃ is selected from the group consisting of: S, T, G, K, A, L, W, Y, E, D, I, M, V, N, P, F, H, and Q; X₅₄ is selected from the group consisting of G, W, S, Y, T, Q, V, R, H, E, M, K, P, N, F, A, L, I, and D; X₅₅ is selected from the group consisting of S, H, G, Q, A, T, L, M, E, W, N, K, R, Y, D, P, I, and F; X₅₆ is selected from the group consisting of: S, M, T, P, K, R, Q, Y, L, G, N, H, I, D, V, F, W, and E; X₅₇ is selected from the group consisting of T, V, S, L, A, I, P, M, R, K, Q, H, W, F, Y, E, and D; and X₅₈ is selected from the group consisting of: I, F, W, Y, V, E, L, M, A, K, R, T, D, N, S, P, H, and G;
an IMGT HCDR3 that comprises the amino acid sequence of A₉₇R₉₈X₉₉X₁₀₀X₁₀₁X₁₀₂X₁₀₃X₁₀₄X₁₀₅X₁₀₆, wherein X₉₉ is selected from the group consisting of R, I, K, L, P, N, S, Y, V, A, G, T, D, Q, H, M, and W; X₁₀₀ is selected from the group consisting of: G, N, H, S, K, Y, M, R, F, L, D, E, T, I, W, V, and P; X₁₀₁ is selected from the group consisting of: E, H, L, A, T, M, R, S, G, N, Y, W, K, I, P, V, and D; X₁₀₂ is selected from the group consisting of G, W, H, Y, K, R, A, F, N, V, M, S, T, E, L, P, I, and D; X₁₀₃ is selected from the group consisting of: A, S, K, Y, G, T, V, P, R, F, W, N, I, Q, L, H, M, D, and E; X₁₀₄ is selected from the group consisting of M, L, A, P, F, G, E, N, T, S, R, H, Q, W, Y, and D; X₁₀₅ is selected from the group consisting of D, I, H, Q, Y, K, T, W, L, R, S, E, M, G, P, F, and V; and X₁₀₆ is selected from the group consisting of: Y, N, A, W, V, M, P, T, Q, R, S, H, L, F, G, and D;
an IMGT LCDR1 that comprises the amino acid sequence of Z₂₇Z₂₈Z₂₉Z₃₀Z₃₁, wherein Z₂₇ is selected from the group consisting of: S, K, R, H, Y, N, I, Q, T, G, M, L, F, V, A, W, P, E, and D; Z₂₈ is selected from the group consisting of: S, K, N, R, P, M, Y, A, I, H, V, Q, W, D, F, E, T, and G; Z₂₉ is selected from the group consisting of I, T, L, A, R, M, G, P, Q, Y, F, H, W, N, E, and D; Z₃₀ is selected from the group consisting of: N, V, R, K, M, A, T, L, W, E, Y, Q, S, H, G, P, F, and D; and Z₃₁ is selected from the group consisting of Y, F, Y, K, R, H, A, N, M, Q, V, P, G, L, T, E, I, and D;
an IMGT LCDR2 that comprises the amino acid sequence of Z₄₉Z₅₀Z₅₁, wherein Z₄₉ is selected from the group consisting of: Y, K, Y, F, I, T, H, M, W, L, Q, A, G, P, V, N, E, and D; Z₅₀ is selected from the group consisting of: T, P, S, I, Q, A, W, F, M, N, G, L, Y, R, K, D, E, and H; and Z₅₁ is selected from the group consisting of S, N, K, R, P, D, L, T, V, Y, H, A, Q, W, I, F, C, E, and G;
an IMGT LCDR3 that comprises the amino acid sequence of Z₈₈Z₈₉Z₉₀Z₉₁Z₉₂Z₉₃Z₉₄Z₉₅Z₉₆, wherein Z₈₈ is selected from the group consisting of: Q, E, L, Y, N, M, P, D, S, V, H, T, W, I, F, and R; Z₈₉ is selected from the group consisting of: Q, H, E, K, V, R, T, G, S, A, L, I, F, M, Y, D, and P; Z₉₀ is selected from the group consisting of F, S, L, K, M, Y V, Q, N, A, R, I, T, H, G, W, D, P, and E; Z₉₁ is selected from the group consisting of: T, S, I, H, N, A, K, G, R, Q, Y, M, F, L, V, P, W, E, and D; Z₉₂ is selected from the group consisting of: S, G, R, A, N, Y, M, W, D, T, F, V, K, Q, H, L, I, E, and P; Z₉₃ is selected from the group consisting of S, K, A, F, R, P, H, T, V, M, L, E, N, Q, W, D, and G; Z₉₄ is selected from the group consisting of: P, W, E, I, Y, T, F, H, L, A, S, C, R, K, N, Q, and V; Z₉₅ is selected from the group consisting of F, L, P, M, V, W, A, N, S, I, G, E, H, Q, and R; and Z₉₆ is selected from the group consisting of T, V, S, H, Y, L, F, I, W, R, Q, M, E, K, G, and D and
wherein the polypeptide molecule does not comprise the amino acid sequence of SEQ ID NOs: 1157, 1182, 1186, 1197, and 1201, and YTS.

Embodiment 66. The polypeptide molecule of embodiment 65, wherein X₂₇ is F; X₂₈ is selected from the group consisting of T and K; X₂₉ is F; X₃₀ is selected from the group consisting of S and H; X₃₁ is selected from the group consisting of T N, R, I, Y, V, H, L, T, S, Q, and D; or X₃₂ is selected from the group consisting of F, K, T, Y, V, R, A, H, and I.

Embodiment 67. The polypeptide molecule of embodiment 65 or 66, wherein X₃₁ is selected from the group consisting of R and I; or X₃₂ is T.

Embodiment 68. The polypeptide molecule of any one of embodiments 65-67, wherein X₅₃ is S; X₅₄ is selected from the group consisting of G, W, S, Y, T, Q, V, R, H, E, M, K, and P; X₅₅ is selected from the group consisting of S and H; X₅₆ is S; X₅₇ is selected from the group consisting of T and V; or X₅₈ is selected from the group consisting of I, F, W, and Y.

Embodiment 69. The polypeptide molecule of any one of embodiments 65-68, wherein X₅₄ is selected from the group consisting of S and T.

Embodiment 70. The polypeptide molecule of any one of embodiments 65-19, wherein X₉₉ is R; X₁₀₀ is G; X₁₀₁ is selected from the group consisting of E, H, L, A, T, M, R, S, G, N, Y, W, K, I, and P; X₁₀₂ is G; X₁₀₃ is A; X₁₀₄ is M; X₁₀₅ is D; or X₁₀₆ is selected from the group consisting of Y, N, A, W, V, M, P, and T.

Embodiment 71. The polypeptide molecule of any one of embodiments 65-70, wherein X₁₀₁ is A.

Embodiment 72. The polypeptide molecule of any one of embodiments 65-71, wherein Z₂₇ is selected from the group consisting of S, K, R, and H; Z₂₈ is selected from the group consisting of S, K, N, R, and P; Z₂₉ is I; Z₃₀ is selected from the group consisting of N and V; or Z₃₁ is selected from the group consisting of Y and F.

Embodiment 73. The polypeptide molecule of any one of embodiments 65-72, wherein Z₂₇ is selected from the group consisting of K and R.

Embodiment 74. The polypeptide molecule of any one of embodiments 65-73, wherein Z₄₉ is selected from the group consisting of Y and K; Z₅₀ is selected from the group consisting of T and P; or Z₅₁ is selected from the group consisting of S, N, K, R, P, D, and L.

Embodiment 75. The polypeptide molecule of any one of embodiments 65-74, wherein Z₈₈ is Q; Z₈₉ is Q; Z₉₀ is F; Z₉₁ is T; Z₉₂ is selected from the group consisting of S, G, and R; Z₉₃ is selected from the group consisting of S, K, A, F, R, P, and H; Z₉₄ is P; Z₉₅ is F; or Z₉₆ is selected from the group consisting of T, V, S, H, and Y.

Embodiment 76. The polypeptide molecule of any one of embodiments 65-75, wherein Z₉₆ is V.

Embodiment 77. A polypeptide molecule that specifically binds to TCRβV, wherein the polypeptide molecule comprises:
an IMGT HCDR1 that comprises the amino acid sequence of G₂₆F₂₇X₂₈F₂₉X₃₀X₃₁X₃₂G₃₃, wherein X₂₈ is selected from the group consisting of T and K; X₃₀ is selected from the group consisting of S and H; X₃₁ is selected from the group consisting of: N, R, I, Y, V, H, L, T, S, Q, and D; and X₃₂ is selected from the group consisting of F, K, T, Y V, R, A, H, and I;
an IMGT HCDR2 that comprises the amino acid sequence of I₅₁S₅₂S₅₃X₅₄X₅₅S₅₆X₅₇X₅₈, wherein X₅₄ is selected from the group consisting of G, W, S, Y, T, Q, V, R, H, E, M, K, and P; X₅₅ is selected from the group consisting of S and H; X₅₇ is selected from the group consisting of T and V; and X₅₈ is selected from the group consisting of: I, F, W, and Y;
an IMGT HCDR3 that comprises the amino acid sequence of A₉₇R₉₈R₉₉G₁₀₀X₁₀₁G₁₀₂A₁₀₃M₁₀₄D₁₀₅X₁₀₆, wherein X₁₀₁ is selected from the group consisting of: E, H, L, A, T, M, R, S, G, N, Y, W, K, I, and P; and X₁₀₆ is selected from the group consisting of: Y, N, A, W, V, M, P, and T;
an IMGT LCDR1 that comprises the amino acid sequence of Z₂₇Z₂₈I₂₉Z₃₀Z₃₁, wherein Z₂₇ is selected from the group consisting of: S, K, R, and H; Z₂₈ is selected from the group consisting of: S, K, N, R, and P; Z₃₀ is selected from the group consisting of: N and V; and Z₃₁ is selected from the group consisting of Y and F;
an IMGT LCDR2 that comprises the amino acid sequence of Z₄₉Z₅₀Z₅₁, wherein Z₄₉ is selected from the group consisting of: Y and K; Z₅₀ is selected from the group consisting of: T and P; and Z₅₁ is selected from the group consisting of S, N, K, R, P, D, and L;
an IMGT LCDR3 that comprises the amino acid sequence of Q₈₈Q₈₉F₉₀T₉₁Z₉₂Z₉₃P₉₄F₉₅Z₉₆, wherein Z₉₂ is selected from the group consisting of: S, G, and R; Z₉₃ is selected from the group consisting of S, K, A, F, R, P, and H; and Z₉₆ is selected from the group consisting of T, V, S, H, and Y; and wherein the polypeptide molecule does not comprise the amino acid sequence of SEQ ID NOs: 1157, 1182, 1186, 1197, and 1201, and YTS.

Embodiment 78. A polypeptide molecule comprising an anti-TCRβV-binding domain, wherein the binding domain comprises:
an IMGT HCDR1 that comprises an amino acid sequence selected from any one of: GFTFSIFG (SEQ ID NO: 1188), GFTFSRTG (SEQ ID NO: 1189), GFTFSIKG (SEQ ID NO: 1190), GFTFSNKG (SEQ ID NO: 1191), GFTFSRFG (SEQ ID NO: 1192), GFTFSRKG (SEQ ID NO: 1193), GFTFSITG (SEQ ID NO: 1194), GFTFSIYG (SEQ ID NO: 1195), and GFTFSNTG (SEQ ID NO: 1196);
an IMGT HCDR2 that comprises an amino acid sequence selected from any one of: ISSGSSTI (SEQ ID NO: 1197), ISSTSSTI (SEQ ID NO: 1198), ISSSSSTI (SEQ ID NO: 1199), and ISSWSSTI (SEQ ID NO: 1200);
an IMGT HCDR3 that comprises an amino acid sequence of ARRGEGAMDY (SEQ ID NO: 1201) or ARRGAGAMDY (SEQ ID NO: 1202);
an IMGT LCDR1 that comprises an amino acid sequence selected from any one of: SSVNY (SEQ ID NO: 1182), RSVNY (SEQ ID NO: 1184), and KSVNY (SEQ ID NO: 1185);
an IMGT LCDR2 that comprises an amino acid sequence of YTS;
an IMGT LCDR3 that comprises an amino acid sequence selected from any one of: QQFTSSPFT (SEQ ID NO: 1157) and QQFTSSPFV (SEQ ID NO: 1159);
wherein each of the HCDR1, HCDR2, HCDR3, LCDR1, LCDR2, and/or LCDR3 independently comprise 0, 1, 2, or 3 amino acid mutations.

Embodiment 79. A polypeptide molecule comprising an anti-TCRβV-binding domain, wherein the binding domain comprises:
an IMGT HCDR1 that comprises an amino acid sequence selected from any one of: GFTFSIFG (SEQ ID NO: 1188), GFTFSRTG (SEQ ID NO: 1189), GFTFSIKG (SEQ ID NO: 1190), GFTFSNKG (SEQ ID NO: 1191), GFTFSRFG (SEQ ID NO: 1192), GFTFSRKG (SEQ ID NO: 1193), GFTFSITG (SEQ ID NO: 1194), GFTFSIYG (SEQ ID NO: 1195), and GFTFSNTG (SEQ ID NO: 1196);
an IMGT HCDR2 that comprises an amino acid sequence selected from any one of: ISSGSSTI (SEQ ID NO: 1197), ISSTSSTI (SEQ ID NO: 1198), ISSSSSTI (SEQ ID NO: 1199), and ISSWSSTI (SEQ ID NO: 1200);
an IMGT HCDR3 that comprises an amino acid sequence of ARRGEGAMDY (SEQ ID NO: 1201) or ARRGAGAMDY (SEQ ID NO: 1202);
an IMGT LCDR1 that comprises an amino acid sequence selected from any one of: SSVNY (SEQ ID NO: 1182), RSVNY (SEQ ID NO: 1184), and KSVNY (SEQ ID NO: 1185);
an IMGT LCDR2 that comprises an amino acid sequence of YTS;
an IMGT LCDR3 that comprises an amino acid sequence selected from any one of: QQFTSSPFT (SEQ ID NO: 1157) and QQFTSSPFV (SEQ ID NO: 1159).

Embodiment 80. The polypeptide molecule of embodiment 79, wherein the binding domain comprises:
(i) the HCDR1, the HCDR2, and the HCDR3 comprising the amino acid sequences of SEQ ID NOs: 1188, 1198, and 1201, respectively, and the LCDR1, the LCDR2, and the LCDR3 comprising the amino acid sequences of SEQ ID NOs: 1184, YTS, and 1159, respectively;
(ii) the HCDR1, the HCDR2, and the HCDR3 comprising the amino acid sequences of SEQ ID NOs: 1188, 1198, and 1201, respectively, and the LCDR1, the LCDR2, and the LCDR3 comprising the amino acid sequences of SEQ ID NOs: 1182, YTS, and 1159, respectively;
(iii) the HCDR1, the HCDR2, and the HCDR3 comprising the amino acid sequences of SEQ ID NOs: 1189, 1199, and 1201, respectively, and the LCDR1, the LCDR2, and the LCDR3 comprising the amino acid sequences of SEQ ID NOs: 1184, YTS, and 1159, respectively;
(iv) the HCDR1, the HCDR2, and the HCDR3 comprising the amino acid sequences of SEQ ID NOs: 1190, 1197, and 1202, respectively, and the LCDR1, the LCDR2, and the LCDR3 comprising the amino acid sequences of SEQ ID NOs: 1185, YTS, and 1159, respectively;
(v) the HCDR1, the HCDR2, and the HCDR3 comprising the amino acid sequences of SEQ ID NOs: 1191, 1199, and 1201, respectively, and the LCDR1, the LCDR2, and the LCDR3 comprising the amino acid sequences of SEQ ID NOs: 1185, YTS, and 1157, respectively;
(vi) the HCDR1, the HCDR2, and the HCDR3 comprising the amino acid sequences of SEQ ID NOs: 1186, 1200, and 1202, respectively, and the LCDR1, the LCDR2, and the LCDR3 comprising the amino acid sequences of SEQ ID NOs: 1182, YTS, and 1159, respectively;
(vii) the HCDR1, the HCDR2, and the HCDR3 comprising the amino acid sequences of SEQ ID NOs: 1188, 1200, and 1201, respectively, and the LCDR1, the LCDR2, and the LCDR3 comprising the amino acid sequences of SEQ ID NOs: 1184, YTS, and 1157, respectively;
(viii) the HCDR1, the HCDR2, and the HCDR3 comprising the amino acid sequences of SEQ ID NOs: 1192, 1200, and 1201, respectively, and the LCDR1, the LCDR2, and the LCDR3 comprising the amino acid sequences of SEQ ID NOs: 1185, YTS, and 1159, respectively;
(ix) the HCDR1, the HCDR2, and the HCDR3 comprising the amino acid sequences of SEQ ID NOs: 1193, 1198, and 1201, respectively, and the LCDR1, the LCDR2, and the LCDR3 comprising the amino acid sequences of SEQ ID NOs: 1184, YTS, and 1159, respectively;
(x) the HCDR1, the HCDR2, and the HCDR3 comprising the amino acid sequences of SEQ ID NOs: 1188, 1200, and 1202, respectively, and the LCDR1, the LCDR2, and the LCDR3 comprising the amino acid sequences of SEQ ID NOs: 1182, YTS, and 1159, respectively;
(xi) the HCDR1, the HCDR2, and the HCDR3 comprising the amino acid sequences of SEQ ID NOs: 1188, 1199, and 1202, respectively, and the LCDR1, the LCDR2, and the LCDR3 comprising the amino acid sequences of SEQ ID NOs: 1182, YTS, and 1159, respectively;
(xii) the HCDR1, the HCDR2, and the HCDR3 comprising the amino acid sequences of SEQ ID NOs: 1191, 1199, and 1202, respectively, and the LCDR1, the LCDR2, and the LCDR3 comprising the amino acid sequences of SEQ ID NOs: 1182, YTS, and 1157, respectively;
(xiii) the HCDR1, the HCDR2, and the HCDR3 comprising the amino acid sequences of SEQ ID NOs: 1192, 1198, and 1201, respectively, and the LCDR1, the LCDR2, and the LCDR3 comprising the amino acid sequences of SEQ ID NOs: 1184, YTS, and 1157, respectively;
(xiv) the HCDR1, the HCDR2, and the HCDR3 comprising the amino acid sequences of SEQ ID NOs: 1194, 1198, and 1202, respectively, and the LCDR1, the LCDR2, and the LCDR3 comprising the amino acid sequences of SEQ ID NOs: 1185, YTS, and 1159, respectively;
(xv) the HCDR1, the HCDR2, and the HCDR3 comprising the amino acid sequences of SEQ ID NOs: 1193, 1197, and 1202, respectively, and the LCDR1, the LCDR2, and the LCDR3 comprising the amino acid sequences of SEQ ID NOs: 1182, YTS, and 1157, respectively;
(xvi) the HCDR1, the HCDR2, and the HCDR3 comprising the amino acid sequences of SEQ ID NOs: 1194, 1200, and 1202, respectively, and the LCDR1, the LCDR2, and the LCDR3 comprising the amino acid sequences of SEQ ID NOs: 1185, YTS, and 1157, respectively;
(xvii) the HCDR1, the HCDR2, and the HCDR3 comprising the amino acid sequences of SEQ ID NOs: 1190, 1199, and 1202, respectively, and the LCDR1, the LCDR2, and the LCDR3 comprising the amino acid sequences of SEQ ID NOs: 1185, YTS, and 1159, respectively;
(xviii) the HCDR1, the HCDR2, and the HCDR3 comprising the amino acid sequences of SEQ ID NOs: 1193, 1200, and 1202, respectively, and the LCDR1, the LCDR2, and the LCDR3 comprising the amino acid sequences of SEQ ID NOs: 1182, YTS, and 1157, respectively;
(xix) the HCDR1, the HCDR2, and the HCDR3 comprising the amino acid sequences of SEQ ID NOs: 1188, 1198, and 1201, respectively, and the LCDR1, the LCDR2, and the LCDR3 comprising the amino acid sequences of SEQ ID NOs: 1184, YTS, and 1157, respectively;
(xx) the HCDR1, the HCDR2, and the HCDR3 comprising the amino acid sequences of SEQ ID NOs: 1195, 1198, and 1202, respectively, and the LCDR1, the LCDR2, and the LCDR3 comprising the amino acid sequences of SEQ ID NOs: 1182, YTS, and 1157, respectively;
(xxi) the HCDR1, the HCDR2, and the HCDR3 comprising the amino acid sequences of SEQ ID NOs: 1189, 1197, and 1201, respectively, and the LCDR1, the LCDR2, and the LCDR3 comprising the amino acid sequences of SEQ ID NOs: 1185, YTS, and 1159, respectively;
(xxii) the HCDR1, the HCDR2, and the HCDR3 comprising the amino acid sequences of SEQ ID NOs: 1196, 1198, and 1202, respectively, and the LCDR1, the LCDR2, and the LCDR3 comprising the amino acid sequences of SEQ ID NOs: 1184, YTS, and 1157, respectively;
(xxiii) the HCDR1, the HCDR2, and the HCDR3 comprising the amino acid sequences of SEQ ID NOs: 1191, 1197, and 1201, respectively, and the LCDR1, the LCDR2, and the LCDR3 comprising the amino acid sequences of SEQ ID NOs: 1185, YTS, and 1157, respectively;
(xxiv) the HCDR1, the HCDR2, and the HCDR3 comprising the amino acid sequences of SEQ ID NOs: 1194, 1198, and 1202, respectively, and the LCDR1, the LCDR2, and the LCDR3 comprising the amino acid sequences of SEQ ID NOs: 1184, YTS, and 1159, respectively;
(xxv) the HCDR1, the HCDR2, and the HCDR3 comprising the amino acid sequences of SEQ ID NOs: 1192, 1198, and 1201, respectively, and the LCDR1, the LCDR2, and the LCDR3 comprising the amino acid sequences of SEQ ID NOs: 1184, YTS, and 1159, respectively;
(xxvi) the HCDR1, the HCDR2, and the HCDR3 comprising the amino acid sequences of SEQ ID NOs: 1194, 1199, and 1202, respectively, and the LCDR1, the LCDR2, and the LCDR3 comprising the amino acid sequences of SEQ ID NOs: 1185, YTS, and 1159, respectively;
(xxvii) the HCDR1, the HCDR2, and the HCDR3 comprising the amino acid sequences of SEQ ID NOs: 1189, 1200, and 1201, respectively, and the LCDR1, the LCDR2, and the LCDR3 comprising the amino acid sequences of SEQ ID NOs: 1182, YTS, and 1159, respectively;
(xxviii) the HCDR1, the HCDR2, and the HCDR3 comprising the amino acid sequences of SEQ ID NOs: 1193, 1200, and 1202, respectively, and the LCDR1, the LCDR2, and the LCDR3 comprising the amino acid sequences of SEQ ID NOs: 1185, YTS, and 1159, respectively;
(xxix) the HCDR1, the HCDR2, and the HCDR3 comprising the amino acid sequences of SEQ ID NOs: 1186, 1200, and 1202, respectively, and the LCDR1, the LCDR2, and the LCDR3 comprising the amino acid sequences of SEQ ID NOs: 1185, YTS, and 1159, respectively;
(xxx) the HCDR1, the HCDR2, and the HCDR3 comprising the amino acid sequences of SEQ ID NOs: 1196, 1198, and 1201, respectively, and the LCDR1, the LCDR2, and the LCDR3 comprising the amino acid sequences of SEQ ID NOs: 1184, YTS, and 1159, respectively;
(xxxi) the HCDR1, the HCDR2, and the HCDR3 comprising the amino acid sequences of SEQ ID NOs: 1196, 1199, and 1202, respectively, and the LCDR1, the LCDR2, and the LCDR3 comprising the amino acid sequences of SEQ ID NOs: 1184, YTS, and 1159, respectively;
(xxxii) the HCDR1, the HCDR2, and the HCDR3 comprising the amino acid sequences of SEQ ID NOs: 1189, 1200, and 1202, respectively, and the LCDR1, the LCDR2, and the LCDR3 comprising the amino acid sequences of SEQ ID NOs: 1184, YTS, and 1159, respectively;
(xxxiii) the HCDR1, the HCDR2, and the HCDR3 comprising the amino acid sequences of SEQ ID NOs: 1191, 1200, and 1202, respectively, and the LCDR1, the LCDR2, and the LCDR3 comprising the amino acid sequences of SEQ ID NOs: 1182, YTS, and 1157, respectively;
(xxxiv) the HCDR1, the HCDR2, and the HCDR3 comprising the amino acid sequences of SEQ ID NOs: 1191, 1197, and 1202, respectively, and the LCDR1, the LCDR2, and the LCDR3 comprising the amino acid sequences of SEQ ID NOs: 1184, YTS, and 1159, respectively;
(xxxv) the HCDR1, the HCDR2, and the HCDR3 comprising the amino acid sequences of SEQ ID NOs: 1189, 1199, and 1201, respectively, and the LCDR1, the LCDR2, and the LCDR3 comprising the amino acid sequences of SEQ ID NOs: 1185, YTS, and 1159, respectively;
(xxxvi) the HCDR1, the HCDR2, and the HCDR3 comprising the amino acid sequences of SEQ ID NOs: 1196, 1199, and 1202, respectively, and the LCDR1, the LCDR2, and the LCDR3 comprising the amino acid sequences of SEQ ID NOs: 1184, YTS, and 1159, respectively; or
(xxxvii) the HCDR1, the HCDR2, and the HCDR3 comprising the amino acid sequences of SEQ ID NOs: 1194, 1199, and 1201, respectively, and the LCDR1, the LCDR2, and the LCDR3 comprising the amino acid sequences of SEQ ID NOs: 1184, YTS, and 1159, respectively.

Embodiment 81. The polypeptide molecule of any one of embodiments 49-80, wherein the polypeptide molecule comprises an immunoglobulin heavy chain variable (VH) region and/or an immunoglobulin light chain variable (VL) region.

Embodiment 82. The polypeptide molecule of embodiment 81, wherein the VH region comprises an amino acid sequence that is at least about 80%, at least about 85%, at least about 90%, at least about 95%, at least about 96%, at least about 97%, at least about 98%, at least about 99%, or 100% identical to any one of SEQ ID NOs: 1116-1147.

Embodiment 83. The polypeptide molecule of embodiment 81, wherein the VL region comprises an amino acid sequence that is at least about 80%, at least about 85%, at least about 90%, at least about 95%, at least about 96%, at least about 97%, at least about 98%, at least about 99%, or 100% identical to any one of SEQ ID NOs: 1104-1112.

Embodiment 84. The polypeptide molecule of embodiment 81, wherein the VH region comprises an amino acid sequence that is at least about 80%, at least about 85%, at least about 90%, at least about 95%, at least about 96%, at least about 97%, at least about 98%, at least about 99%, or 100% identical to any one of SEQ ID NOs: 1116-1147; and the VL region comprises an amino acid sequence that is at least about 80%, at least about 85%, at least about 90%, at least about 95%, at least about 96%, at least about 97%, at least about 98%, at least about 99%, or 100% identical to any one of SEQ ID NOs: 1104-1112.

Embodiment 85. The polypeptide molecule of any one of embodiments 49-84, wherein the TCRβV-binding domain is an scFv, and the scFv comprises an amino acid sequence that is at least about 80%, at least about 85%, at least about 90%, at least about 95%, at least about 96%, at least about 97%, at least about 98%, at least about 99%, or 100% identical to any one of SEQ ID NOs: 1203-1277.

Embodiment 86. The polypeptide molecule of any one of embodiments 49-84, wherein the TCRβV-binding domain comprises a Fab or a CrossFab, and the Fab or CrossFab comprises (i) a light chain sequence that is at least about 80%, at least about 85%, at least about 90%, at least about 95%, at least about 96%, at least about 97%, at least about 98%, at least about 99%, or 100% identical to any one of SEQ ID NOs: 1280-1292; and (ii) a heavy chain sequence that is at least about 80%, at least about 85%, at least about 90%, at least about 95%, at least about 96%, at least about 97%, at least about 98%, at least about 99%, or 100% identical to any one of SEQ ID NOs: 1293-1328.

Embodiment 87. A polypeptide molecule comprising a first binding domain and a second binding domain, wherein:
(i) the first binding domain is the TCRβV-binding domain of any one of embodiments 1-86; and
(ii) the second binding domain is a tumor-associated antigen (TAA)-binding domain.

Embodiment 88. The polypeptide molecule of embodiment 87, wherein the second binding domain comprises
(i) an immunoglobulin heavy chain variable (VH) region comprising a heavy chain CDR1 (HCDR1), a heavy chain CDR2 (HCDR2), and a heavy chain CDR3 (HCDR3); and/or
(ii) an immunoglobulin light chain variable (VL) region comprising a light chain CDR1 (LCDR1), a light chain CDR2 (LCDR2), and a light chain CDR3 (LCDR3).

Embodiment 89. The polypeptide molecule of embodiment 87 or 88, wherein the TAA-binding domain is a CD19-binding domain.

Embodiment 90. The polypeptide molecule of embodiment 88 or 89, wherein the CD19-binding domain comprises:
(i) the HCDR1, the HCDR2, and the HCDR3 comprising the amino acid sequences of SEQ ID NOs: 1353, 1355, and 1357, respectively, and an LCDR1, an LCDR2, and an LCDR3 comprising the amino acid sequences of SEQ ID NOs: 1347, 1349, and 1351, respectively; or
(ii) the HCDR1, the HCDR2, and the HCDR3 comprising the amino acid sequences of SEQ ID NOs: 1354, 1356, and 1358, respectively, and an LCDR1, an LCDR2, and an LCDR3 comprising the amino acid sequences of SEQ ID NOs: 1348, 1350, and 1352, respectively; and wherein the CDRs are numbered with reference to the Kabat numbering scheme.

Embodiment 91. The polypeptide molecule of embodiment 88 or 89, wherein the CD19-binding domain comprises a VH region and a VL region, wherein the VH region comprises an amino acid sequence that is at least about 80%, at least about 85%, at least about 90%, at least about 95%, at least about 96%, at least about 97%, at least about 98%, at least about 99%, or 100% identical to SEQ ID NO: 1345 or SEQ ID NO: 1346; and wherein the VL region comprises an amino acid sequence that is at least about 80%, at least about 85%, at least about 90%, at least about 95%, at least about 96%, at least about 97%, at least about 98%, at least about 99%, or 100% identical to SEQ ID NO: 1343 or SEQ ID NO: 1344.

Embodiment 92. The polypeptide molecule of any one of embodiments 88-91, wherein the second binding domain comprises a Fab or CrossFab comprising:
(i) a heavy chain comprising an amino acid sequence that is at least about 80%, at least about 85%, at least about 90%, at least about 95%, at least about 96%, at least about 97%, at least about 98%, at least about 99%, or 100% identical to SEQ ID NO: 1370 or SEQ ID NO: 1371; and
(ii) a light chain comprising an amino acid sequence that is at least about 80%, at least about 85%, at least about 90%, at least about 95%, at least about 97%, at least about 98%, at least about 99%, or 100% identical to SEQ ID NO: 1368 or SEQ ID NO: 1369.

Embodiment 93. The polypeptide molecule of any one of embodiments 88-91, wherein the second binding domain is an scFv comprising an amino acid sequence that is at least about 80%, at least about 85%, at least about 90%, at least about 95%, at least about 96%, at least about 97%, at least about 98%, at least about 99%, or 100% identical to SEQ ID NO: 1367.

Embodiment 94. The polypeptide molecule of any one of embodiments 87-93, wherein the polypeptide molecule further comprises a half-life extension moiety.

Embodiment 95. The polypeptide molecule of embodiment 94, wherein the half-life extension moiety is a fragment crystallizable (Fc) region, and the Fc region comprises two polypeptides.

Embodiment 96. The polypeptide molecule of embodiment 95, wherein the first binding domain is linked to one of the polypeptides of the Fc region and the second binding domain is linked to the other polypeptide of the Fc region.

Embodiment 97. The polypeptide molecule of embodiment 95, wherein the first binding domain and the second binding domain are linked to the same polypeptides of the Fc region.

Embodiment 98. The polypeptide molecule of any one of embodiments 94 to 97, wherein the Fc region has a knobs-into-holes format, wherein the first polypeptide of the Fc region comprises one or more hole mutations, and wherein second polypeptide of the Fc region comprises one or more knob mutations.

Embodiment 99. The polypeptide molecule of embodiment 98, wherein the first polypeptide of the Fc region comprises mutations T366S, L368A and Y407V according to EU numbering, and wherein the second polypeptide of the Fc region comprises a mutation T366W according to EU numbering.

Embodiment 100. The polypeptide molecule of embodiment 98 or 99, wherein the first polypeptide of the Fc region comprising the one or more hole mutations further comprises a mutation Y349C according to EU numbering, wherein the second polypeptide of the Fc region comprising the one or more knob mutations further comprises a mutation S354C according to EU numbering, and wherein the Fc region comprises an engineered disulfide bond formed between these two cysteines.

Embodiment 101. The polypeptide molecule of any one of embodiments 95 to 100, wherein one or both of the polypeptides of the Fc region comprise mutations L234A, L235A, and P329G according to EU numbering.

Embodiment 102. The polypeptide molecule of any one of embodiments 95 to 101, wherein one or both of the polypeptides of the Fc region comprise the mutation N297G according to EU numbering.

Embodiment 103. The polypeptide molecule of any one of embodiments 95 to 102, wherein one or both of the polypeptides of the Fc region comprise mutations H435R and Y436F according to EU numbering.

Embodiment 104. The polypeptide molecule of any one of embodiments 95 to 103, wherein the first polypeptide of the Fc region comprises an amino acid sequence that is at least about 80%, at least about 85%, at least about 90%, at least about 95%, at least about 97%, at least about 98%, at least about 99%, or 100% identical to SEQ ID NO: 1412, and wherein the second polypeptide of the Fc region comprises an amino acid sequence that is at least about 80%, at least about 85%, at least about 90%, at least about 95%, at least about 97%, at least about 98%, at least about 99%, or 100% identical to SEQ ID NO: 1414.

Embodiment 105. The polypeptide molecule of any one of embodiments 95 to 103, wherein the first polypeptide of the Fc region comprises an amino acid sequence that is at least about 80%, at least about 85%, at least about 90%, at least about 95%, at least about 97%, at least about 98%, at least about 99%, or 100% identical to SEQ ID NO: 1413, and wherein the second polypeptide of the Fc region comprises an amino acid sequence that is at least about 80%, at least about 85%, at least about 90%, at least about 95%, at least about 97%, at least about 98%, at least about 99%, or 100% identical to SEQ ID NO: 1415.

Embodiment 106. The polypeptide molecule of any one of embodiments 87-105, wherein the polypeptide molecule comprises three polypeptides.

Embodiment 107. The polypeptide molecule of embodiment 106, wherein:
(i) a first polypeptide comprises an amino acid sequence that is at least about 80%, at least about 85%, at least about 90%, at least about 95%, at least about 97%, at least about 98%, at least about 99%, or 100% identical to SEQ ID NO: 1412, a second polypeptide comprises an amino acid sequence that is at least about 80%, at least about 85%, at least about 90%, at least about 95%, at least about 97%, at least about 98%, at least about 99%, or 100% identical to SEQ ID NO: 1416, and a third polypeptide comprises an amino acid sequence that is at least about 80%, at least about 85%, at least about 90%, at least about 95%, at least about 97%, at least about 98%, at least about 99%, or 100% identical to SEQ ID NO: 1417;
(ii) a first polypeptide comprises an amino acid sequence that is at least about 80%, at least about 85%, at least about 90%, at least about 95%, at least about 97%, at least about 98%, at least about 99%, or 100% identical to SEQ ID NO: 1412, a second polypeptide comprises an amino acid sequence that is at least about 80%, at least about 85%, at least about 90%, at least about 95%, at least about 97%, at least about 98%, at least about 99%, or 100% identical to SEQ ID NO: 1369, and a third polypeptide comprises an amino acid sequence that is at least about 80%, at least about 85%, at least about 90%, at least about 95%, at least about 97%, at least about 98%, at least about 99%, or 100% identical to SEQ ID NO: 1418;
(iii) a first polypeptide comprises an amino acid sequence that is at least about 80%, at least about 85%, at least about 90%, at least about 95%, at least about 97%, at least about 98%, at least about 99%, or 100% identical to SEQ ID NO: 1412, a second polypeptide comprises an amino acid sequence that is at least about 80%, at least about 85%, at least about 90%, at least about 95%, at least about 97%, at least about 98%, at least about 99%, or 100% identical to SEQ ID NO: 1419, and a third polypeptide comprises an amino acid sequence that is at least about 80%, at least about 85%, at least about 90%, at least about 95%, at least about 97%, at least about 98%, at least about 99%, or 100% identical to SEQ ID NO: 1417;
(iv) a first polypeptide comprises an amino acid sequence that is at least about 80%, at least about 85%, at least about 90%, at least about 95%, at least about 97%, at least about 98%, at least about 99%, or 100% identical to SEQ ID NO: 1412, a second polypeptide comprises an amino acid sequence that is at least about 80%, at least about 85%, at least about 90%, at least about 95%, at least about 97%, at least about 98%, at least about 99%, or 100% identical to SEQ ID NO: 1420, and a third polypeptide comprises an amino acid sequence that is at least about 80%, at least about 85%, at least about 90%, at least about 95%, at least about 97%, at least about 98%, at least about 99%, or 100% identical to SEQ ID NO: 1417;
(v) a first polypeptide comprises an amino acid sequence that is at least about 80%, at least about 85%, at least about 90%, at least about 95%, at least about 97%, at least about 98%, at least about 99%, or 100% identical to SEQ ID NO: 1412, a second polypeptide comprises an amino acid sequence that is at least about 80%, at least about 85%, at least about 90%, at least about 95%, at least about 97%, at least about 98%, at least about 99%, or 100% identical to SEQ ID NO: 1369, and a third polypeptide comprises an amino acid sequence that is at least about 80%, at least about 85%, at least about 90%, at least about 95%, at least about 97%, at least about 98%, at least about 99%, or 100% identical to SEQ ID NO: 1421;
(vi) a first polypeptide comprises an amino acid sequence that is at least about 80%, at least about 85%, at least about 90%, at least about 95%, at least about 97%, at least about 98%, at least about 99%, or 100% identical to SEQ ID NO: 1412, a second polypeptide comprises an amino acid sequence that is at least about 80%, at least about 85%, at least about 90%, at least about 95%, at least about 97%, at least about 98%, at least about 99%, or 100% identical to SEQ ID NO: 1369, and a third polypeptide comprises an amino acid sequence that is at least about 80%, at least about 85%, at least about 90%, at least about 95%, at least about 97%, at least about 98%, at least about 99%, or 100% identical to SEQ ID NO: 1422; or
(vii) a first polypeptide comprises an amino acid sequence that is at least about 80%, at least about 85%, at least about 90%, at least about 95%, at least about 97%, at least about 98%, at least about 99%, or 100% identical to SEQ ID NO: 1412, a second polypeptide comprises an amino acid sequence that is at least about 80%, at least about 85%, at least about 90%, at least about 95%, at least about 97%, at least about 98%, at least about 99%, or 100% identical to SEQ ID NO: 1369, and a third polypeptide comprises an amino acid sequence that is at least about 80%, at least about 85%, at least about 90%, at least about 95%, at least about 97%, at least about 98%, at least about 99%, or 100% identical to SEQ ID NO: 1423.

Embodiment 108. A recombinant nucleic acid molecule encoding the polypeptide molecule of any one of embodiments 1-107.

Embodiment 109. A vector comprising the recombinant nucleic acid molecule of embodiment 108.

Embodiment 110. A pharmaceutical composition comprising (i) the polypeptide molecule of any one of embodiments 1 to 107, the recombinant nucleic acid molecule of embodiment 108, or the vector of embodiment 109, and (ii) a pharmaceutically acceptable carrier, diluent, or excipient.

Embodiment 111. A method of expanding a T cell population, wherein the method comprises contacting the T cell population with the polypeptide molecule of any one of embodiments 1 to 107, the recombinant nucleic acid molecule of embodiment 108, the vector of embodiment 109, or the pharmaceutical composition of embodiment 110, wherein the T cell population expresses TCRβV on the cell surface, and wherein the TCRβV-binding domain of the polypeptide molecule specifically binds to the TCRβV.

Embodiment 112. A method of depleting target cells in a subject, comprising administering to the subject an effective amount of the polypeptide molecule of any one of embodiments 1 to 107, the recombinant nucleic acid molecule of embodiment 108, the vector of embodiment 109, or the pharmaceutical composition of embodiment 110, wherein the target cells are tumor cells.

Embodiment 113. A method of depleting target cells in a subject, comprising administering to the subject an effective amount of the polypeptide molecule of any one of embodiments 1 to 107, the recombinant nucleic acid molecule of embodiment 108, the vector of embodiment 109, or the pharmaceutical composition of embodiment 110, wherein the target cells are B cells.

Embodiment 114. A method of treating a disease, disorder, or condition in a subject in need thereof, comprising administering to the subject an effective amount of the polypeptide molecule of any one of embodiments 1 to 107, the recombinant nucleic acid molecule of embodiment 108, the vector of embodiment 109, or the pharmaceutical composition of embodiment 110.

Embodiment 115. The method of embodiment 114, wherein the disease, disorder, or condition is cancer.

Embodiment 116. The method of embodiment 114, wherein the disease, disorder, or condition is an autoimmune disease.

### Section 3.

Embodiment 1. A polypeptide molecule that specifically binds to T cell receptor beta variable region (TCRβV), wherein the polypeptide molecule comprises:
a heavy chain CDR1 (HCDR1) that comprises the amino acid sequence of GX₀X₁X₂X₃X₄X₅YIH (SEQ ID NO: 74), wherein X₀ is selected from the group consisting of Y, E, H, and P, X₁ is selected from the group consisting of S, A, N, Q, E, G, H, I, L, K, M, P, T, and V, X₂ is selected from the group consisting of F and I, X₃ is selected from the group consisting of T, A, R, N, Q, E, G, H, I, L, K, M, F, S, W, Y, and V, X₄ is selected from the group consisting of T, Q, H, L, F, S, and Y, and X₅ is selected from the group consisting of Y, A, G, I, L, M, F, T, W, and V;
a heavy chain CDR2 (HCDR2) that comprises the amino acid sequence of X₆X₇X₈PX₉SX₁₀X₁₁X₁₂X₁₃YX₁₄X₁₅KFKG (SEQ ID NO: 75), wherein X₆ is selected from the group consisting of W, R, and D, X₇ is selected from the group consisting of F, A, R, N, Q, E, G, H, L, K, M, T, Y, and V, X₅ is selected from the group consisting of F, R, S, and Y, X₉ is selected from the group consisting of G, R, Q, H, I, L, K, M, F, S, W, and Y, X₁₀ is selected from the group consisting of G, A, R, N, D, Q, E, H, L, K, M, S, T, W, and Y, X₁₁ is selected from the group consisting of N, R, and K, X₁₂ is selected from the group consisting of I, A, R, N, D, Q, E, L, K, M, F, P, S, T, W, Y, and V, X₁₃ is selected from the group consisting of K, A, N, Q, E, H, L, M, F, S, T, W, and V, X₁₄ is selected from the group consisting of N, D, Q, E, G, H, I, K, M, F, P, S, T, W, Y, and V, and X₁₅ is selected from the group consisting of E, A, R, N, D, Q, G, H, I, L, K, M, F, P, S, T, W, Y, and V;
a heavy chain CDR3 (HCDR3) that comprises the amino acid sequence of AX₁₆X₁₇YX₁₈X₁₉X₂₀X₂₁VX₂₂DX₂₃ (SEQ ID NO: 76), wherein X₁₆ is selected from the group consisting of G and A, X₁₇ is selected from the group consisting of S, G and A, X₁₈ is selected from the group consisting of Y and W, X₁₉ is selected from the group consisting of S, A, and T, X₂₀ is selected from the group consisting of Y and F, X₂₁ is selected from the group consisting of D and N, X₂₂ is selected from the group consisting of L, I, and M, and X₂₃ is selected from the group consisting of Y and W, wherein the HCDR3 does not comprise the amino acid sequence of SYYSYDVLDY (SEQ ID NO: 70);
a light chain CDR1 (LCDR1) that comprises the amino acid sequence of KASQZ₀Z₁Z₂Z₃Z₄Z₅Z₆ (SEQ ID NO:77), wherein Z₀ is selected from the group consisting of A, R, N, D, Q, E, G, H, I, L, M, F, P, S, T, W, Y, and V, Z₁ is selected from the group consisting of V, A, D, Q, G, H, L, K, M, F, P, S, T, W, and Y, Z₂ is selected from the group consisting of G, A, R, N, D, Q, E, H, I, L, K, M, S, W, and V, Z₃ is selected from the group consisting of I, A, N, Q, E, G, H, M, F, S, W, Y, and V, Z₄ is selected from the group consisting of N, K, and S, Z₅ is selected from the group consisting of V, A, N, D, Q, E, G, L, F, S, and T, and Z₆ is selected from the group consisting of V and T;
a light chain CDR2 (LCDR2) that comprises the amino acid sequence of Z₇SZ₈HZ₉Z₁₀Z₁₁ (SEQ ID NO: 78), wherein Z₇ is selected from the group consisting of S, A, G, H, and V, Z₈ is selected from the group consisting of S, N, G, M, F, T, W, and Y, Z₉ is selected from the group consisting of R, A, N, I, L, K, P, T, and V, Z₁₀ is selected from the group consisting of Y and W, and Zu is selected from the group consisting of S, R, N, Q, G, H, I, K, F, T, W, Y, and V; and
a light chain CDR3 (LCDR3) that comprises the amino acid sequence of Z₁₂Z₁₃FZ₁₄Z₁₅Z₁₆Z₁₇Z₁₈Z₁₉ (SEQ ID NO: 79), wherein Z₁₂ is selected from the group consisting of Q, A, N, E, I, K, Y, and V, Z₁₃ is selected from the group consisting of N, Q, E, H, and M, Z₁₄ is selected from the group consisting of A, R, Q, G, H, I, K, M, F, S, T, W, Y, and V, Z₁₅ is selected from the group consisting of S, A, R, N, D, Q, G, I, L, K, M, F, T, W, and V, Z₁₆ is selected from the group consisting of Y, A, R, N, Q, E, I, L, and V, Z₁₇ is selected from the group consisting of A, N, D, G, H, P, S, T, and V, Z₁₈ is selected from the group consisting of L, I, M, and V, and Z₁₉ is selected from the group consisting of A, R, N, Q, E, G, H, I, L, M, F, S, T, W, and V, and wherein the LCDR3 does not comprise the amino acid sequence of QQFKSYPLT (SEQ ID NO: 73).

Embodiment 2. A polypeptide molecule that specifically binds to T cell receptor beta variable region (TCRβV), wherein the polypeptide molecule comprises:
a heavy chain CDR1 (HCDR1) that comprises the amino acid sequence of GX₀X₁X₂X₃X₄X₅YIH (SEQ ID NO: 74), wherein X₂ is a hydrophobic amino acid, and X₅ is a hydrophobic amino acid;
a heavy chain CDR2 (HCDR2) comprises the amino acid sequence of X₆X₇X₈PX₉SX₁₀X₁₁X₁₂X₁₃YX₁₄X₁₅KFKG (SEQ ID NO: 75), wherein X₈ is a hydrophobic amino acid or an aromatic amino acid;
a heavy chain CDR3 (HCDR3) that comprises the amino acid sequence of AX₁₆X₁₇YX₁₈X₁₉X₂₀X₂₁VX₂₂DX₂₃ (SEQ ID NO: 76), wherein X₁₆ is a small and flexible amino acid, X₁₇ is a small and flexible amino acid, X₁₈ is a hydrophobic amino acid, X₂₁ is a polar amino acid or a charged amino acid, X₂₂ is a hydrophobic amino acid, and X₂₃ is a hydrophobic amino acid or an aromatic amino acid, wherein the HCDR3 does not comprise the amino acid sequence of SYYSYDVLDY (SEQ ID NO: 70);
a light chain CDR1 (LCDR1) comprises the amino acid sequence of KASQZ₀Z₁Z₂Z₃Z₄Z₅Z₆ (SEQ ID NO: 77), wherein Z₃ is an aliphatic amino acid or a charged amino acid, Z₄ is a polar amino acid, and Z₆ is a branched-chain amino acid;
a light chain CDR2 (LCDR2) comprises the amino acid sequence of Z₇SZ₈HZ₉Z₁₀Z₁₁ (SEQ ID NO: 78), wherein Z₁₀ is a hydrophobic amino acid or an aromatic amino acid; and
a light chain CDR3 (LCDR3) comprises the amino acid sequence of Z₁₂Z₁₃FZ₁₄Z₁₅Z₁₆Z₁₇Z₁₈Z₁₉ (SEQ ID NO: 79), wherein Z₁₆ is a hydrophobic amino acid, and Z₁₈ is a hydrophobic amino acid or an aliphatic amino acid, wherein the LCDR3 does not comprise the amino acid sequence of QQFKSYPLT (SEQ ID NO: 73).

Embodiment 3. A polypeptide molecule that specifically binds to T cell receptor beta variable region (TCRβV), wherein the polypeptide molecule comprises:
(i) a heavy chain CDR1 (HCDR1), a heavy chain CDR2 (HCDR2), and a heavy chain CDR3 (HCDR3) comprising amino acid sequences selected from the group consisting of SEQ ID NOs: 112, 126, 136, 145, and 147, and SEQ ID NOs: 69, 129, and 142, and SEQ ID NOs: 70 and 137; and/or
(ii) a light chain CDR1 (LCDR1), a light chain CDR2 (LCDR2), and a light chain CDR3 (LCDR3) comprising amino acid sequences selected from the group consisting of SEQ ID NOs: 71, 127, and 134, and SEQ ID NOs: 72, 128, and 144, and SEQ ID NOs: 73, 135 and 141; and wherein the CDRs are numbered with reference to the Kabat numbering scheme.

Embodiment 4. The polypeptide molecule of embodiment 0, wherein the polypeptide molecule comprises:
(i) the HCDR1, the HCDR2, and the HCDR3 comprising the amino acid sequences of SEQ ID NOs: 136, 69, and 137, respectively, and the LCDR1, the LCDR2, and the LCDR3 comprising the amino acid sequences of SEQ ID NOs: 134, 72, and 135, respectively;
(ii) the HCDR1, the HCDR2, and the HCDR3 comprising the amino acid sequences of SEQ ID NOs: 112, 142, and 137, respectively, and the LCDR1, the LCDR2, and the LCDR3 comprising the amino acid sequences of SEQ ID NOs: 134, 72, and 141, respectively;
(iii) the HCDR1, the HCDR2, and the HCDR3 comprising the amino acid sequences of SEQ ID NOs: 112, 69, and 137, respectively, and the LCDR1, the LCDR2, and the LCDR3 comprising the amino acid sequences of SEQ ID NOs: 134, 144, and 135, respectively;
(iv) the HCDR1, the HCDR2, and the HCDR3 comprising the amino acid sequences of SEQ ID NOs: 112, 142, and 137, respectively, and the LCDR1, the LCDR2, and the LCDR3 comprising the amino acid sequences of SEQ ID NOs: 134, 72, and 135, respectively;
(v) the HCDR1, the HCDR2, and the HCDR3 comprising the amino acid sequences of SEQ ID NOs: 147, 69, and 137, respectively, and the LCDR1, the LCDR2, and the LCDR3 comprising the amino acid sequences of SEQ ID NOs: 134, 72, and 135, respectively;
(vi) the HCDR1, the HCDR2, and the HCDR3 comprising the amino acid sequences of SEQ ID NOs: 147, 142, and 137, respectively, and the LCDR1, the LCDR2, and the LCDR3 comprising the amino acid sequences of SEQ ID NOs: 71, 128, and 135, respectively;
(vii) the HCDR1, the HCDR2, and the HCDR3 comprising the amino acid sequences of SEQ ID NOs: 136, 142, and 137, respectively, and the LCDR1, the LCDR2, and the LCDR3 comprising the amino acid sequences of SEQ ID NOs: 134, 128, and 135, respectively;
(viii) the HCDR1, the HCDR2, and the HCDR3 comprising the amino acid sequences of SEQ ID NOs: 112, 142, and 137, respectively, and the LCDR1, the LCDR2, and the LCDR3 comprising the amino acid sequences of SEQ ID NOs: 71, 144, and 135, respectively;
(ix) the HCDR1, the HCDR2, and the HCDR3 comprising the amino acid sequences of SEQ ID NOs: 112, 142, and 137, respectively, and the LCDR1, the LCDR2, and the LCDR3 comprising the amino acid sequences of SEQ ID NOs: 134, 144, and 135, respectively;
(x) the HCDR1, the HCDR2, and the HCDR3 comprising the amino acid sequences of SEQ ID NOs: 136, 142, and 137, respectively, and the LCDR1, the LCDR2, and the LCDR3 comprising the amino acid sequences of SEQ ID NOs: 134, 144, and 135, respectively;
(xi) the HCDR1, the HCDR2, and the HCDR3 comprising the amino acid sequences of SEQ ID NOs: 145, 69, and 137, respectively, and the LCDR1, the LCDR2, and the LCDR3 comprising the amino acid sequences of SEQ ID NOs: 134, 128, and 141, respectively;
(xii) the HCDR1, the HCDR2, and the HCDR3 comprising the amino acid sequences of SEQ ID NOs: 147, 69, and 137, respectively, and the LCDR1, the LCDR2, and the LCDR3 comprising the amino acid sequences of SEQ ID NOs: 71, 144, and 141, respectively;
(xiii) the HCDR1, the HCDR2, and the HCDR3 comprising the amino acid sequences of SEQ ID NOs: 147, 69, and 137, respectively, and the LCDR1, the LCDR2, and the LCDR3 comprising the amino acid sequences of SEQ ID NOs: 134, 72, and 135, respectively;
(xiv) the HCDR1, the HCDR2, and the HCDR3 comprising the amino acid sequences of SEQ ID NOs: 112, 142, and 137, respectively, and the LCDR1, the LCDR2, and the LCDR3 comprising the amino acid sequences of SEQ ID NOs: 134, 128, and 141, respectively;
(xv) the HCDR1, the HCDR2, and the HCDR3 comprising the amino acid sequences of SEQ ID NOs: 136, 69, and 137, respectively, and the LCDR1, the LCDR2, and the LCDR3 comprising the amino acid sequences of SEQ ID NOs: 134, 128, and 141, respectively;
(xvi) the HCDR1, the HCDR2, and the HCDR3 comprising the amino acid sequences of SEQ ID NOs: 147, 142, and 137, respectively, and the LCDR1, the LCDR2, and the LCDR3 comprising the amino acid sequences of SEQ ID NOs: 134, 144, and 135, respectively;
(xvii) the HCDR1, the HCDR2, and the HCDR3 comprising the amino acid sequences of SEQ ID NOs: 136, 142, and 137, respectively, and the LCDR1, the LCDR2, and the LCDR3 comprising the amino acid sequences of SEQ ID NOs: 71, 144, and 135, respectively; or
(xviii) the HCDR1, the HCDR2, and the HCDR3 comprising the amino acid sequences of SEQ ID NOs: 147, 142, and 137, respectively, and the LCDR1, the LCDR2, and the LCDR3 comprising the amino acid sequences of SEQ ID NOs: 134, 144, and 141, respectively.

Embodiment 5. A polypeptide molecule specifically binds to T cell receptor beta variable region (TCRβV), wherein the polypeptide molecule comprises:
(i) a heavy chain CDR1 (HCDR1), a heavy chain CDR2 (HCDR2), and a heavy chain CDR3 (HCDR3) comprising amino acid sequences selected from the group consisting of SEQ ID NOs: 114, 125, 131, 139, 146, and 148, SEQ ID NOs: 115, 132, and 143, and SEQ ID NOs: 116, 133, and 140; and/or
(ii) a light chain CDR1 (LCDR1), a light chain CDR2 (LCDR2), and a light chain CDR3 (LCDR3) comprising amino acid sequences selected from the group consisting of SEQ ID NOs: 113, 130, and 138, SSS, and SEQ ID NOs: 73, 135, and 141 and wherein the CDRs are numbered with reference to the IMGT numbering scheme.

Embodiment 6. The polypeptide molecule of embodiment 0, wherein the polypeptide molecule comprises:
(i) the HCDR1, the HCDR2, and the HCDR3 comprising the amino acid sequences of SEQ ID NOs: 139, 115, and 140, respectively, and the LCDR1, the LCDR2, and the LCDR3 comprising the amino acid sequences of SEQ ID NOs: 138, SSS, and 135, respectively;
(ii) the HCDR1, the HCDR2, and the HCDR3 comprising the amino acid sequences of SEQ ID NOs: 114, 143, and 140, respectively, and the LCDR1, the LCDR2, and the LCDR3 comprising the amino acid sequences of SEQ ID NOs: 138, SSS, and 141, respectively;
(iii) the HCDR1, the HCDR2, and the HCDR3 comprising the amino acid sequences of SEQ ID NOs: 114, 115, and 140, respectively, and the LCDR1, the LCDR2, and the LCDR3 comprising the amino acid sequences of SEQ ID NOs: 138, SSS, and 135, respectively;
(iv) the HCDR1, the HCDR2, and the HCDR3 comprising the amino acid sequences of SEQ ID NOs: 114, 143, and 140, respectively, and the LCDR1, the LCDR2, and the LCDR3 comprising the amino acid sequences of SEQ ID NOs: 138, SSS, and 135, respectively;
(v) the HCDR1, the HCDR2, and the HCDR3 comprising the amino acid sequences of SEQ ID NOs: 148, 115, and 140, respectively, and the LCDR1, the LCDR2, and the LCDR3 comprising the amino acid sequences of SEQ ID NOs: 138, SSS, and 135, respectively;
(vi) the HCDR1, the HCDR2, and the HCDR3 comprising the amino acid sequences of SEQ ID NOs: 148, 143, and 140, respectively, and the LCDR1, the LCDR2, and the LCDR3 comprising the amino acid sequences of SEQ ID NOs: 113, SSS, and 135, respectively;
(vii) the HCDR1, the HCDR2, and the HCDR3 comprising the amino acid sequences of SEQ ID NOs: 139, 143, and 140, respectively, and the LCDR1, the LCDR2, and the LCDR3 comprising the amino acid sequences of SEQ ID NOs: 138, SSS, and 135, respectively;
(viii) the HCDR1, the HCDR2, and the HCDR3 comprising the amino acid sequences of SEQ ID NOs: 114, 143, and 140, respectively, and the LCDR1, the LCDR2, and the LCDR3 comprising the amino acid sequences of SEQ ID NOs: 113, SSS, and 135, respectively;
(ix) the HCDR1, the HCDR2, and the HCDR3 comprising the amino acid sequences of SEQ ID NOs: 114, 143, and 140, respectively, and the LCDR1, the LCDR2, and the LCDR3 comprising the amino acid sequences of SEQ ID NOs: 138, SSS, and 135, respectively;
(x) the HCDR1, the HCDR2, and the HCDR3 comprising the amino acid sequences of SEQ ID NOs: 139, 143, and 140, respectively, and the LCDR1, the LCDR2, and the LCDR3 comprising the amino acid sequences of SEQ ID NOs: 138, SSS, and 135, respectively;
(xi) the HCDR1, the HCDR2, and the HCDR3 comprising the amino acid sequences of SEQ ID NOs: 146, 115, and 140, respectively, and the LCDR1, the LCDR2, and the LCDR3 comprising the amino acid sequences of SEQ ID NOs: 138, SSS, and 141, respectively;
(xii) the HCDR1, the HCDR2, and the HCDR3 comprising the amino acid sequences of SEQ ID NOs: 148, 115, and 140, respectively, and the LCDR1, the LCDR2, and the LCDR3 comprising the amino acid sequences of SEQ ID NOs: 138, SSS, and 141, respectively;
(xiii) the HCDR1, the HCDR2, and the HCDR3 comprising the amino acid sequences of SEQ ID NOs: 148, 115, and 140, respectively, and the LCDR1, the LCDR2, and the LCDR3 comprising the amino acid sequences of SEQ ID NOs: 138, SSS, and 135, respectively;
(xiv) the HCDR1, the HCDR2, and the HCDR3 comprising the amino acid sequences of SEQ ID NOs: 114, 143, and 140, respectively, and the LCDR1, the LCDR2, and the LCDR3 comprising the amino acid sequences of SEQ ID NOs: 138, SSS, and 141, respectively;
(xv) the HCDR1, the HCDR2, and the HCDR3 comprising the amino acid sequences of SEQ ID NOs: 139, 115, and 140, respectively, and the LCDR1, the LCDR2, and the LCDR3 comprising the amino acid sequences of SEQ ID NOs: 138, SSS, and 141, respectively;
(xvi) the HCDR1, the HCDR2, and the HCDR3 comprising the amino acid sequences of SEQ ID NOs: 148, 143, and 140, respectively, and the LCDR1, the LCDR2, and the LCDR3 comprising the amino acid sequences of SEQ ID NOs: 138, SSS, and 135, respectively;
(xvii) the HCDR1, the HCDR2, and the HCDR3 comprising the amino acid sequences of SEQ ID NOs: 139, 143, and 140, respectively, and the LCDR1, the LCDR2, and the LCDR3 comprising the amino acid sequences of SEQ ID NOs: 138, SSS, and 135, respectively; or
(xviii) the HCDR1, the HCDR2, and the HCDR3 comprising the amino acid sequences of SEQ ID NOs: 148, 143, and 140, respectively, and the LCDR1, the LCDR2, and the LCDR3 comprising the amino acid sequences of SEQ ID NOs: 138, SSS, and 141, respectively.

Embodiment 7. The polypeptide molecule of any one of embodiments [00913] to [00918], wherein the TCRβV is TCRβV6-5.

Embodiment 8. A polypeptide molecule specifically binds to T cell receptor beta variable region (TCRβV), wherein the polypeptide molecule comprises:
a heavy chain CDR1 (HCDR1) comprises the amino acid sequence of GB₀B₁B₂B₃B₄B₅GMB₆ (SEQ ID NO: 1329), wherein B₀ is selected from the group consisting of I, L, M, F, W, and Y, B₁ is selected from the group consisting of T, A, R, N, D, Q, E, G, H, I, L, K, M, F, S, W, Y, and V, B₂ is selected from the group consisting of F, W, and Y, B₃ is selected from the group consisting of R, N, Q, G, H, K, M, F, P, S, W, Y, and V, B₄ is selected from the group consisting of N, A, R, D, Q, G, H, I, L, K, M, F, S, T, W, Y, and V, B₅ is selected from the group consisting of F, A, R, N, Q, E, G, H, I, L, K, M, T, W, Y, and V, and B₆ is selected from the group consisting of H, N, F, and W;
a heavy chain CDR2 (HCDR2) comprises the amino acid sequence of YISB₇B₈B₉B₁₀B₁₁B₁₂B₁₃YB₁₄B₁₅B₁₆LKG (SEQ ID NO: 1330), wherein B7 is selected from the group consisting of S and T, B₈ is selected from the group consisting of G, A, R, N, Q, E, H, I, L, K, M, F, P, S, T, W, Y, and V, B₉ is selected from the group consisting of S, A, Q, G, H, and T, B₁₀ is selected from the group consisting of S, R, Q, G, H, K, M, and T, B₁₁ is selected from the group consisting of T, A, L, S, and V, B12 is selected from the group consisting of I, A, R, N, D, E, H, L, K, M, F, P, S, T, W, Y, and V, B13 is selected from the group consisting of Y, A, R, Q, G, H, I, L, K, M, F, T, and V, B14 is selected from the group consisting of A, R, N, D, Q, E, G, H, I, L, K, M, F, P, S, T, W, Y, and V, B15 is selected from the group consisting of D, R, N, Q, E, G, H, I, K, P, T, and Y, and B16 is selected from the group consisting of T, A, R, N, D, Q, E, G, H, I, L, K, M, F, P, S, W, Y, and V;
a heavy chain CDR3 (HCDR3) comprises the amino acid sequence of ARRB₁₇B₁₈B₁₉AB₂₀B₂₁B₂₂ (SEQ ID NO: 1331), wherein B₁₇ is selected from the group consisting of N, G, H, and S, B₁₈ is selected from the group consisting of E, A, R, N, D, G, H, I, L, K, M, P, S, T, W, Y, and V, Bis is selected from the group consisting of G and H, B20 is selected from the group consisting of M and L, B21 is selected from the group consisting of D and K, and B₂₂ is selected from the group consisting of Y, A, R, N, Q, G, H, L, M, P, S, T, W, and V, wherein the HCDR3 does not comprise the amino acid sequence of RGEGAMDY (SEQ ID NO: 1180);
a light chain CDR1 (LCDR1) comprises the amino acid sequence of RAJ₀J₁J₂J₃J₄J₅J₆Y (SEQ ID NO: 1332), wherein J₀ is selected from the group consisting of S, A, R, N, D, Q, G, H, I, L, M, P, T, W, Y, and V, J₁ is selected from the group consisting of S, A, R, N, D, Q, E, G, H, I, L, K, M, F, P, T, W, Y, and V, J₂ is selected from the group consisting of S, A, R, N, D, Q, E, G, H, I, K, M, F, P, T, W, Y, and V, J₃ is selected from the group consisting of V, I, and T, J₄ is selected from the group consisting of N, A, R, K, M, and V, J₅ is selected from the group consisting of R, K, F, and Y, and J₆ is selected from the group consisting of I, A, Q, L, M, S, and T;
a light chain CDR2 (LCDR2) comprises the amino acid sequence of J₇J₈J₉J₁₀J₁₁J₁₂J₁₃ (SEQ ID NO: 1333), wherein J₇ is selected from the group consisting of H, L, K, M, F, T, W, and Y, J₈ is selected from the group consisting of A, I, S, and T, J₉ is selected from the group consisting of S, A, R, N, D, Q, E, G, H, I, L, K, F, T, W, Y, and V, J₁₀ is selected from the group consisting of N, A, R, D, E, G, H, I, L, K, M, F, S, W, Y, and V, J₁₁ is selected from the group consisting of L, A, R, N, H, K, F, P, W, and V; J₁₂ is selected from the group consisting of A, R, Q, G, H, I, K, F, P, T, and V, and J₁₃ is selected from the group consisting of P, R, N, D, Q, E, G, H, I, L, M, F, S, T, W, Y, and V; and
a light chain CDR3 (LCDR3) comprises the amino acid sequence of QJ₁₄J₁₅J₁₆J₁₇J₁₈PJ₁₉J₂₀ (SEQ ID NO: 1334), wherein J₁₄ is selected from the group consisting of Q and E, J₁₈ is selected from the group consisting of F, A, Q, L, K, M, S, and Y, J₁₆ is selected from the group consisting of T, I, and S, J₁₇ is selected from the group consisting of S, A, R, N, D, Q, G, H, I, L, K, M, F, T, W, Y, and V, J₁₈ is selected from the group consisting of S, A, R, N, Q, E, H, L, K, M, F, P, T, W, and V, J₁₉ is selected from the group consisting of F, L, M, P, and V, and J₂₀ is selected from the group consisting of T, R, Q, E, H, I, L, K, M, F, S, W, Y, and V, wherein the LCDR3 does not comprise the amino acid sequence of QQFTSSPFT (SEQ ID NO: 1157).

Embodiment 9. A polypeptide molecule that specifically binds to T cell receptor beta variable region (TCRβV), wherein the polypeptide molecule comprises:
a heavy chain CDR1 (HCDR1) comprises the amino acid sequence of GB₀B₁B₂B₃B₄B₅GMB₆ (SEQ ID NO: 1329), wherein B₂ is a hydrophobic amino acid or an aromatic amino acid;
a heavy chain CDR2 (HCDR2) comprises the amino acid sequence of YISB₇B8B9B₁₀B₁₁B12B13YB₁₄B₁₅B₁₆LKG (SEQ ID NO: 1330), wherein B7 is a small and flexible amino acid or a polar amino acid, B₉ is small and flexible amino acid or a polar amino acid or a non-charged amino acid, and B₁₀ is small and flexible amino acid or an amino acid capable of forming hydrogen bonds;
a heavy chain CDR3 (HCDR3) comprises the amino acid sequence of ARRB₁₇B₁₈B₁₉AB₂₀B₂₁B₂₂ (SEQ ID NO: 1331), wherein B₁₇ is a polar amino acid, B₂₀ is a hydrophobic amino acid or an aliphatic amino acid, and B21 is a polar amino acid or a charged amino acid, wherein the HCDR3 does not comprise the amino acid sequence of RGEGAMDY (SEQ ID NO: 1180);
a light chain CDR1 (LCDR1) comprises the amino acid sequence of RAJ₀J₁J₂J₃J₄J₅J₆Y (SEQ ID NO: 1332), wherein J₃ is a branched-chain amino acid;
a light chain CDR2 (LCDR2) comprises the amino acid sequence of J₇J₈J₉J₁₀J₁₁J₁₂J₁₃ (SEQ ID NO: 1333) and
a light chain CDR3 (LCDR3) comprises the amino acid sequence of QJ₁₄J₁₅J₁₆J₁₇J₁₈PJ₁₉J₂₀ (SEQ ID NO: 1334), wherein J₁₄ is a polar amino acid or a charged amino acid, and J₁₉ is a hydrophobic amino acid, wherein the LCDR3 does not comprise the amino acid sequence of QQFTSSPFT (SEQ ID NO: 1157).

Embodiment 10. A polypeptide molecule that specifically binds to T cell receptor beta variable region (TCRβV),
wherein the polypeptide molecule comprises:
   (i) a heavy chain CDR1 (HCDR1), a heavy chain CDR2 (HCDR2), and a heavy chain CDR3 (HCDR3) comprising amino acid sequences selected from the group consisting of SEQ ID NOs: 1160, 1161, 1162, 1163, 1164, 1165, 1166, 1167, 1168, 1169, and 1170, SEQ ID NOs: 1171, 1172, 1173, 1174, 1175, 1176, 1177, 1178, and 1179, and SEQ ID NOs: 1180, and 1181; and/or
   (ii) a light chain CDR1 (LCDR1), a light chain CDR2 (LCDR2), and a light chain CDR3 (LCDR3) comprising amino acid sequences selected from the group consisting of SEQ ID NOs: 1148, 1149, 1150, 1151, 1152, 1153, 1154, and 1155, SEQ ID NO: 1156, and SEQ ID NOs: 1157, 1158, and 1159; and
wherein the CDRs are numbered with reference to the Kabat numbering scheme.

Embodiment 11. The polypeptide molecule of embodiment [00922], wherein the polypeptide molecule comprises:
(i) the HCDR1, the HCDR2, and the HCDR3 comprising the amino acid sequences of SEQ ID NOs: 1162, 1173, and 1180, respectively, and the LCDR1, the LCDR2, and the LCDR3 comprising the amino acid sequences of SEQ ID NOs: 1151, 1156, and 1159, respectively;
(ii) the HCDR1, the HCDR2, and the HCDR3 comprising the amino acid sequences of SEQ ID NOs: 1162, 1173, and 1180, respectively, and the LCDR1, the LCDR2, and the LCDR3 comprising the amino acid sequences of SEQ ID NOs: 1148, 1156, and 1159, respectively;
(iii) the HCDR1, the HCDR2, and the HCDR3 comprising the amino acid sequences of SEQ ID NOs: 1163, 1174, and 1180, respectively, and the LCDR1, the LCDR2, and the LCDR3 comprising the amino acid sequences of SEQ ID NOs: 1151, 1156, and 1159, respectively;
(iv) the HCDR1, the HCDR2, and the HCDR3 comprising the amino acid sequences of SEQ ID NOs: 1164, 1171, and 1181, respectively, and the LCDR1, the LCDR2, and the LCDR3 comprising the amino acid sequences of SEQ ID NOs: 1152, 1156, and 1159, respectively;
(v) the HCDR1, the HCDR2, and the HCDR3 comprising the amino acid sequences of SEQ ID NOs: 1165, 1175, and 1180, respectively, and the LCDR1, the LCDR2, and the LCDR3 comprising the amino acid sequences of SEQ ID NOs: 1153, 1156, and 1157, respectively;
(vi) the HCDR1, the HCDR2, and the HCDR3 comprising the amino acid sequences of SEQ ID NOs: 1160, 1176, and 1181, respectively, and the LCDR1, the LCDR2, and the LCDR3 comprising the amino acid sequences of SEQ ID NOs: 1154, 1156, and 1159, respectively;
(vii) the HCDR1, the HCDR2, and the HCDR3 comprising the amino acid sequences of SEQ ID NOs: 1162, 1177, and 1180, respectively, and the LCDR1, the LCDR2, and the LCDR3 comprising the amino acid sequences of SEQ ID NOs: 1151, 1156, and 1157, respectively;
(viii) the HCDR1, the HCDR2, and the HCDR3 comprising the amino acid sequences of SEQ ID NOs: 1166, 1176, and 1180, respectively, and the LCDR1, the LCDR2, and the LCDR3 comprising the amino acid sequences of SEQ ID NOs: 1153, 1156, and 1159, respectively;
(ix) the HCDR1, the HCDR2, and the HCDR3 comprising the amino acid sequences of SEQ ID NOs: 1167, 1173, and 1180, respectively, and the LCDR1, the LCDR2, and the LCDR3 comprising the amino acid sequences of SEQ ID NOs: 1155, 1156, and 1159, respectively;
(x) the HCDR1, the HCDR2, and the HCDR3 comprising the amino acid sequences of SEQ ID NOs: 1162, 1177, and 1181, respectively, and the LCDR1, the LCDR2, and the LCDR3 comprising the amino acid sequences of SEQ ID NOs: 1154, 1156, and 1159, respectively;
(xi) the HCDR1, the HCDR2, and the HCDR3 comprising the amino acid sequences of SEQ ID NOs: 1162, 1174, and 1181, respectively, and the LCDR1, the LCDR2, and the LCDR3 comprising the amino acid sequences of SEQ ID NOs: 1154, 1156, and 1159, respectively;
(xii) the HCDR1, the HCDR2, and the HCDR3 comprising the amino acid sequences of SEQ ID NOs: 1165, 1174, and 1181, respectively, and the LCDR1, the LCDR2, and the LCDR3 comprising the amino acid sequences of SEQ ID NOs: 1154, 1156, and 1157, respectively;
(xiii) the HCDR1, the HCDR2, and the HCDR3 comprising the amino acid sequences of SEQ ID NOs: 1166, 1178, and 1180, respectively, and the LCDR1, the LCDR2, and the LCDR3 comprising the amino acid sequences of SEQ ID NOs: 1151, 1156, and 1157, respectively;
(xiv) the HCDR1, the HCDR2, and the HCDR3 comprising the amino acid sequences of SEQ ID NOs: 1168, 1173, and 1181, respectively, and the LCDR1, the LCDR2, and the LCDR3 comprising the amino acid sequences of SEQ ID NOs: 1153, 1156, and 1159, respectively;
(xv) the HCDR1, the HCDR2, and the HCDR3 comprising the amino acid sequences of SEQ ID NOs: 1167, 1171, and 1181, respectively, and the LCDR1, the LCDR2, and the LCDR3 comprising the amino acid sequences of SEQ ID NOs: 1154, 1156, and 1157, respectively;
(xvi) the HCDR1, the HCDR2, and the HCDR3 comprising the amino acid sequences of SEQ ID NOs: 1168, 1177, and 1181, respectively, and the LCDR1, the LCDR2, and the LCDR3 comprising the amino acid sequences of SEQ ID NOs: 1153, 1156, and 1157, respectively;
(xvii) the HCDR1, the HCDR2, and the HCDR3 comprising the amino acid sequences of SEQ ID NOs: 1164, 1174, and 1181, respectively, and the LCDR1, the LCDR2, and the LCDR3 comprising the amino acid sequences of SEQ ID NOs: 1153, 1156, and 1159, respectively;
(xviii) the HCDR1, the HCDR2, and the HCDR3 comprising the amino acid sequences of SEQ ID NOs: 1167, 1177, and 1181, respectively, and the LCDR1, the LCDR2, and the LCDR3 comprising the amino acid sequences of SEQ ID NOs: 1154, 1156, and 1157, respectively;
(xix) the HCDR1, the HCDR2, and the HCDR3 comprising the amino acid sequences of SEQ ID NOs: 1162, 1173, and 1180, respectively, and the LCDR1, the LCDR2, and the LCDR3 comprising the amino acid sequences of SEQ ID NOs: 1151, 1156, and 1157, respectively;
(xx) the HCDR1, the HCDR2, and the HCDR3 comprising the amino acid sequences of SEQ ID NOs: 1169, 1173, and 1181, respectively, and the LCDR1, the LCDR2, and the LCDR3 comprising the amino acid sequences of SEQ ID NOs: 1154, 1156, and 1157, respectively;
(xxi) the HCDR1, the HCDR2, and the HCDR3 comprising the amino acid sequences of SEQ ID NOs: 1163, 1179, and 1180, respectively, and the LCDR1, the LCDR2, and the LCDR3 comprising the amino acid sequences of SEQ ID NOs: 1152, 1156, and 1159, respectively;
(xxii) the HCDR1, the HCDR2, and the HCDR3 comprising the amino acid sequences of SEQ ID NOs: 1170, 1173, and 1181, respectively, and the LCDR1, the LCDR2, and the LCDR3 comprising the amino acid sequences of SEQ ID NOs: 1151, 1156, and 1157, respectively;
(xxiii) the HCDR1, the HCDR2, and the HCDR3 comprising the amino acid sequences of SEQ ID NOs: 1165, 1179, and 1180, respectively, and the LCDR1, the LCDR2, and the LCDR3 comprising the amino acid sequences of SEQ ID NOs: 1152, 1156, and 1157, respectively;
(xxiv) the HCDR1, the HCDR2, and the HCDR3 comprising the amino acid sequences of SEQ ID NOs: 1168, 1173, and 1181, respectively, and the LCDR1, the LCDR2, and the LCDR3 comprising the amino acid sequences of SEQ ID NOs: 1155, 1156, and 1159, respectively;
(xxv) the HCDR1, the HCDR2, and the HCDR3 comprising the amino acid sequences of SEQ ID NOs: 1166, 1178, and 1180, respectively, and the LCDR1, the LCDR2, and the LCDR3 comprising the amino acid sequences of SEQ ID NOs: 1151, 1156, and 1159, respectively;
(xxvi) the HCDR1, the HCDR2, and the HCDR3 comprising the amino acid sequences of SEQ ID NOs: 1168, 1175, and 1181, respectively, and the LCDR1, the LCDR2, and the LCDR3 comprising the amino acid sequences of SEQ ID NOs: 1152, 1156, and 1159, respectively;
(xxvii) the HCDR1, the HCDR2, and the HCDR3 comprising the amino acid sequences of SEQ ID NOs: 1163, 1177, and 1180, respectively, and the LCDR1, the LCDR2, and the LCDR3 comprising the amino acid sequences of SEQ ID NOs: 1154, 1156, and 1159, respectively;
(xxviii) the HCDR1, the HCDR2, and the HCDR3 comprising the amino acid sequences of SEQ ID NOs: 1167, 1177, and 1181, respectively, and the LCDR1, the LCDR2, and the LCDR3 comprising the amino acid sequences of SEQ ID NOs: 1153, 1156, and 1159, respectively;
(xxix) the HCDR1, the HCDR2, and the HCDR3 comprising the amino acid sequences of SEQ ID NOs: 1160, 1177, and 1181, respectively, and the LCDR1, the LCDR2, and the LCDR3 comprising the amino acid sequences of SEQ ID NOs: 1153, 1156, and 1159, respectively;
(xxx) the HCDR1, the HCDR2, and the HCDR3 comprising the amino acid sequences of SEQ ID NOs: 1170, 1173, and 1180, respectively, and the LCDR1, the LCDR2, and the LCDR3 comprising the amino acid sequences of SEQ ID NOs: 1151, 1156, and 1159, respectively;
(xxxi) the HCDR1, the HCDR2, and the HCDR3 comprising the amino acid sequences of SEQ ID NOs: 1170, 1174, and 1181, respectively, and the LCDR1, the LCDR2, and the LCDR3 comprising the amino acid sequences of SEQ ID NOs: 1151, 1156, and 1159, respectively;
(xxxii) the HCDR1, the HCDR2, and the HCDR3 comprising the amino acid sequences of SEQ ID NOs: 1163, 1176, and 1181, respectively, and the LCDR1, the LCDR2, and the LCDR3 comprising the amino acid sequences of SEQ ID NOs: 1151, 1156, and 1159, respectively;
(xxxiii) the HCDR1, the HCDR2, and the HCDR3 comprising the amino acid sequences of SEQ ID NOs: 1165, 1176, and 1181, respectively, and the LCDR1, the LCDR2, and the LCDR3 comprising the amino acid sequences of SEQ ID NOs: 1154, 1156, and 1157, respectively;
(xxxiv) the HCDR1, the HCDR2, and the HCDR3 comprising the amino acid sequences of SEQ ID NOs: 1165, 1179, and 1181, respectively, and the LCDR1, the LCDR2, and the LCDR3 comprising the amino acid sequences of SEQ ID NOs: 1151, 1156, and 1159, respectively;
(xxxv) the HCDR1, the HCDR2, and the HCDR3 comprising the amino acid sequences of SEQ ID NOs: 1163, 1174, and 1180, respectively, and the LCDR1, the LCDR2, and the LCDR3 comprising the amino acid sequences of SEQ ID NOs: 1152, 1156, and 1159, respectively;
(xxxvi) the HCDR1, the HCDR2, and the HCDR3 comprising the amino acid sequences of SEQ ID NOs: 1170, 1175, and 1181, respectively, and the LCDR1, the LCDR2, and the LCDR3 comprising the amino acid sequences of SEQ ID NOs: 1151, 1156, and 1159, respectively; or
(xxxvii) the HCDR1, the HCDR2, and the HCDR3 comprising the amino acid sequences of SEQ ID NOs: 1168, 1174, and 1180, respectively, and the LCDR1, the LCDR2, and the LCDR3 comprising the amino acid sequences of SEQ ID NOs: 1155, 1156, and 1159, respectively.

Embodiment 12. A polypeptide molecule that specifically binds to T cell receptor beta variable region (TCRβV),
wherein the polypeptide molecule comprises:
   (i) a heavy chain CDR1 (HCDR1), a heavy chain CDR2 (HCDR2), and a heavy chain CDR3 (HCDR3) comprising amino acid sequences selected from the group consisting of SEQ ID NOs: 1186, 1187, 1188, 1189, 1190, 1191, 1192, 1193, 1194, 1195, and 1196, SEQ ID NOs: 1197, 1198, 1199, and 1200, and SEQ ID NOs: 1201, and 1202; and/or
   (ii) a light chain CDR1 (LCDR1), a light chain CDR2 (LCDR2), and a light chain CDR3 (LCDR3) comprising amino acid sequences selected from the group consisting of SEQ ID NOs: 1182, 1183, 1184, and 1185, YTS, and SEQ ID NOs: 1157, 1158, and 1159; and
wherein the CDRs are numbered with reference to the IMGT numbering scheme.

Embodiment 13. The polypeptide molecule of embodiment [00924], wherein the polypeptide molecule comprises:
(i) the HCDR1, the HCDR2, and the HCDR3 comprising the amino acid sequences of SEQ ID NOs: 1188, 1198, and 1201, respectively, and the LCDR1, the LCDR2, and the LCDR3 comprising the amino acid sequences of SEQ ID NOs: 1184, YTS, and 1159, respectively;
(ii) the HCDR1, the HCDR2, and the HCDR3 comprising the amino acid sequences of SEQ ID NOs: 1188, 1198, and 1201, respectively, and the LCDR1, the LCDR2, and the LCDR3 comprising the amino acid sequences of SEQ ID NOs: 1182, YTS, and 1159, respectively;
(iii) the HCDR1, the HCDR2, and the HCDR3 comprising the amino acid sequences of SEQ ID NOs: 1189, 1199, and 1201, respectively, and the LCDR1, the LCDR2, and the LCDR3 comprising the amino acid sequences of SEQ ID NOs: 1184, YTS, and 1159, respectively;
(iv) the HCDR1, the HCDR2, and the HCDR3 comprising the amino acid sequences of SEQ ID NOs: 1190, 1197, and 1202, respectively, and the LCDR1, the LCDR2, and the LCDR3 comprising the amino acid sequences of SEQ ID NOs: 1185, YTS, and 1159, respectively;
(v) the HCDR1, the HCDR2, and the HCDR3 comprising the amino acid sequences of SEQ ID NOs: 1191, 1199, and 1201, respectively, and the LCDR1, the LCDR2, and the LCDR3 comprising the amino acid sequences of SEQ ID NOs: 1185, YTS, and 1157, respectively;
(vi) the HCDR1, the HCDR2, and the HCDR3 comprising the amino acid sequences of SEQ ID NOs: 1186, 1200, and 1202, respectively, and the LCDR1, the LCDR2, and the LCDR3 comprising the amino acid sequences of SEQ ID NOs: 1182, YTS, and 1159, respectively;
(vii) the HCDR1, the HCDR2, and the HCDR3 comprising the amino acid sequences of SEQ ID NOs: 1188, 1200, and 1201, respectively, and the LCDR1, the LCDR2, and the LCDR3 comprising the amino acid sequences of SEQ ID NOs: 1184, YTS, and 1157, respectively;
(viii) the HCDR1, the HCDR2, and the HCDR3 comprising the amino acid sequences of SEQ ID NOs: 1192, 1200, and 1201, respectively, and the LCDR1, the LCDR2, and the LCDR3 comprising the amino acid sequences of SEQ ID NOs: 1185, YTS, and 1159, respectively;
(ix) the HCDR1, the HCDR2, and the HCDR3 comprising the amino acid sequences of SEQ ID NOs: 1193, 1198, and 1201, respectively, and the LCDR1, the LCDR2, and the LCDR3 comprising the amino acid sequences of SEQ ID NOs: 1184, YTS, and 1159, respectively;
(x) the HCDR1, the HCDR2, and the HCDR3 comprising the amino acid sequences of SEQ ID NOs: 1188, 1200, and 1202, respectively, and the LCDR1, the LCDR2, and the LCDR3 comprising the amino acid sequences of SEQ ID NOs: 1182, YTS, and 1159, respectively;
(xi) the HCDR1, the HCDR2, and the HCDR3 comprising the amino acid sequences of SEQ ID NOs: 1188, 1199, and 1202, respectively, and the LCDR1, the LCDR2, and the LCDR3 comprising the amino acid sequences of SEQ ID NOs: 1182, YTS, and 1159, respectively;
(xii) the HCDR1, the HCDR2, and the HCDR3 comprising the amino acid sequences of SEQ ID NOs: 1191, 1199, and 1202, respectively, and the LCDR1, the LCDR2, and the LCDR3 comprising the amino acid sequences of SEQ ID NOs: 1182, YTS, and 1157, respectively;
(xiii) the HCDR1, the HCDR2, and the HCDR3 comprising the amino acid sequences of SEQ ID NOs: 1192, 1198, and 1201, respectively, and the LCDR1, the LCDR2, and the LCDR3 comprising the amino acid sequences of SEQ ID NOs: 1184, YTS, and 1157, respectively;
(xiv) the HCDR1, the HCDR2, and the HCDR3 comprising the amino acid sequences of SEQ ID NOs: 1194, 1198, and 1202, respectively, and the LCDR1, the LCDR2, and the LCDR3 comprising the amino acid sequences of SEQ ID NOs: 1185, YTS, and 1159, respectively;
(xv) the HCDR1, the HCDR2, and the HCDR3 comprising the amino acid sequences of SEQ ID NOs: 1193, 1197, and 1202, respectively, and the LCDR1, the LCDR2, and the LCDR3 comprising the amino acid sequences of SEQ ID NOs: 1182, YTS, and 1157, respectively;
(xvi) the HCDR1, the HCDR2, and the HCDR3 comprising the amino acid sequences of SEQ ID NOs: 1194, 1200, and 1202, respectively, and the LCDR1, the LCDR2, and the LCDR3 comprising the amino acid sequences of SEQ ID NOs: 1185, YTS, and 1157, respectively;
(xvii) the HCDR1, the HCDR2, and the HCDR3 comprising the amino acid sequences of SEQ ID NOs: 1190, 1199, and 1202, respectively, and the LCDR1, the LCDR2, and the LCDR3 comprising the amino acid sequences of SEQ ID NOs: 1185, YTS, and 1159, respectively;
(xviii) the HCDR1, the HCDR2, and the HCDR3 comprising the amino acid sequences of SEQ ID NOs: 1193, 1200, and 1202, respectively, and the LCDR1, the LCDR2, and the LCDR3 comprising the amino acid sequences of SEQ ID NOs: 1182, YTS, and 1157, respectively;
(xix) the HCDR1, the HCDR2, and the HCDR3 comprising the amino acid sequences of SEQ ID NOs: 1188, 1198, and 1201, respectively, and the LCDR1, the LCDR2, and the LCDR3 comprising the amino acid sequences of SEQ ID NOs: 1184, YTS, and 1157, respectively;
(xx) the HCDR1, the HCDR2, and the HCDR3 comprising the amino acid sequences of SEQ ID NOs: 1195, 1198, and 1202, respectively, and the LCDR1, the LCDR2, and the LCDR3 comprising the amino acid sequences of SEQ ID NOs: 1182, YTS, and 1157, respectively;
(xxi) the HCDR1, the HCDR2, and the HCDR3 comprising the amino acid sequences of SEQ ID NOs: 1189, 1197, and 1201, respectively, and the LCDR1, the LCDR2, and the LCDR3 comprising the amino acid sequences of SEQ ID NOs: 1185, YTS, and 1159, respectively;
(xxii) the HCDR1, the HCDR2, and the HCDR3 comprising the amino acid sequences of SEQ ID NOs: 1196, 1198, and 1202, respectively, and the LCDR1, the LCDR2, and the LCDR3 comprising the amino acid sequences of SEQ ID NOs: 1184, YTS, and 1157, respectively;
(xxiii) the HCDR1, the HCDR2, and the HCDR3 comprising the amino acid sequences of SEQ ID NOs: 1191, 1197, and 1201, respectively, and the LCDR1, the LCDR2, and the LCDR3 comprising the amino acid sequences of SEQ ID NOs: 1185, YTS, and 1157, respectively;
(xxiv) the HCDR1, the HCDR2, and the HCDR3 comprising the amino acid sequences of SEQ ID NOs: 1194, 1198, and 1202, respectively, and the LCDR1, the LCDR2, and the LCDR3 comprising the amino acid sequences of SEQ ID NOs: 1184, YTS, and 1159, respectively;
(xxv) the HCDR1, the HCDR2, and the HCDR3 comprising the amino acid sequences of SEQ ID NOs: 1192, 1198, and 1201, respectively, and the LCDR1, the LCDR2, and the LCDR3 comprising the amino acid sequences of SEQ ID NOs: 1184, YTS, and 1159, respectively;
(xxvi) the HCDR1, the HCDR2, and the HCDR3 comprising the amino acid sequences of SEQ ID NOs: 1194, 1199, and 1202, respectively, and the LCDR1, the LCDR2, and the LCDR3 comprising the amino acid sequences of SEQ ID NOs: 1185, YTS, and 1159, respectively;
(xxvii) the HCDR1, the HCDR2, and the HCDR3 comprising the amino acid sequences of SEQ ID NOs: 1189, 1200, and 1201, respectively, and the LCDR1, the LCDR2, and the LCDR3 comprising the amino acid sequences of SEQ ID NOs: 1182, YTS, and 1159, respectively;
(xxviii) the HCDR1, the HCDR2, and the HCDR3 comprising the amino acid sequences of SEQ ID NOs: 1193, 1200, and 1202, respectively, and the LCDR1, the LCDR2, and the LCDR3 comprising the amino acid sequences of SEQ ID NOs: 1185, YTS, and 1159, respectively;
(xxix) the HCDR1, the HCDR2, and the HCDR3 comprising the amino acid sequences of SEQ ID NOs: 1186, 1200, and 1202, respectively, and the LCDR1, the LCDR2, and the LCDR3 comprising the amino acid sequences of SEQ ID NOs: 1185, YTS, and 1159, respectively;
(xxx) the HCDR1, the HCDR2, and the HCDR3 comprising the amino acid sequences of SEQ ID NOs: 1196, 1198, and 1201, respectively, and the LCDR1, the LCDR2, and the LCDR3 comprising the amino acid sequences of SEQ ID NOs: 1184, YTS, and 1159, respectively;
(xxxi) the HCDR1, the HCDR2, and the HCDR3 comprising the amino acid sequences of SEQ ID NOs: 1196, 1199, and 1202, respectively, and the LCDR1, the LCDR2, and the LCDR3 comprising the amino acid sequences of SEQ ID NOs: 1184, YTS, and 1159, respectively;
(xxxii) the HCDR1, the HCDR2, and the HCDR3 comprising the amino acid sequences of SEQ ID NOs: 1189, 1200, and 1202, respectively, and the LCDR1, the LCDR2, and the LCDR3 comprising the amino acid sequences of SEQ ID NOs: 1184, YTS, and 1159, respectively;
(xxxiii) the HCDR1, the HCDR2, and the HCDR3 comprising the amino acid sequences of SEQ ID NOs: 1191, 1200, and 1202, respectively, and the LCDR1, the LCDR2, and the LCDR3 comprising the amino acid sequences of SEQ ID NOs: 1182, YTS, and 1157, respectively;
(xxxiv) the HCDR1, the HCDR2, and the HCDR3 comprising the amino acid sequences of SEQ ID NOs: 1191, 1197, and 1202, respectively, and the LCDR1, the LCDR2, and the LCDR3 comprising the amino acid sequences of SEQ ID NOs: 1184, YTS, and 1159, respectively;
(xxxv) the HCDR1, the HCDR2, and the HCDR3 comprising the amino acid sequences of SEQ ID NOs: 1189, 1199, and 1201, respectively, and the LCDR1, the LCDR2, and the LCDR3 comprising the amino acid sequences of SEQ ID NOs: 1185, YTS, and 1159, respectively;
(xxxvi) the HCDR1, the HCDR2, and the HCDR3 comprising the amino acid sequences of SEQ ID NOs: 1196, 1199, and 1202, respectively, and the LCDR1, the LCDR2, and the LCDR3 comprising the amino acid sequences of SEQ ID NOs: 1184, YTS, and 1159, respectively; or
(xxxvii) the HCDR1, the HCDR2, and the HCDR3 comprising the amino acid sequences of SEQ ID NOs: 1194, 1199, and 1201, respectively, and the LCDR1, the LCDR2, and the LCDR3 comprising the amino acid sequences of SEQ ID NOs: 1184, YTS, and 1159, respectively.

Embodiment 14. The polypeptide molecule of any one of embodiments [00919] to [00925], wherein the TCRβV is TCRβV12-3.

Embodiment 15. The polypeptide molecule of any one of embodiments [00913] to [00919], wherein the polypeptide molecule binds to human TCRβV6-5 with an increased binding affinity when compared to that of a polypeptide molecule comprising a HCDR1, a HCDR2, and a HCDR3 comprising the amino acid sequences of SEQ ID NOs: 112, 69, and 70, respectively, and a LCDR1, a LCDR2, and a LCDR3 comprising the amino acid sequences of SEQ ID NOs: 71, 72, and 73, respectively, according to Kabat.

Embodiment 16. The polypeptide molecule of any one of embodiments [00919] to [00926], wherein the polypeptide molecule binds to human TCRβV12-3 with an increased binding affinity when compared to that of a polypeptide molecule comprising a HCDR1, a HCDR2, and a HCDR3 comprising the amino acid sequences of SEQ ID NOs: 1160, 1171, and 1180, respectively, and a LCDR1, a LCDR2, and a LCDR3 comprising the amino acid sequences of SEQ ID NOs: 1148, 1156, and 1157, respectively, according to Kabat.

Embodiment 17. The polypeptide molecule of embodiment [00927] or [00928] wherein the binding affinity is increased by at least 10%, 30%, 50%, 70%, 90%, 1 time, 2 times, 3 times, 4 times, 5 times, or 10 times.

Embodiment 18. The polypeptide molecule of any one of embodiments [00913] to [00919], wherein the polypeptide molecule binds to cynomolgus monkey TCRβV6-5 with an increased binding affinity when compared to that of a polypeptide molecule comprising a HCDR1, a HCDR2, and a HCDR3 comprising the amino acid sequences of SEQ ID NOs: 112, 69, and 70, respectively, and a LCDR1, a LCDR2, and a LCDR3 comprising the amino acid sequences of SEQ ID NOs: 71, 72, and 73, respectively, according to Kabat.

Embodiment 19. The polypeptide molecule of any one of embodiments [00919] to [00926], wherein the polypeptide molecule binds to cynomolgus monkey TCRβV12-3 with an increased binding affinity when compared to that of a polypeptide molecule comprising a HCDR1, a HCDR2, and a HCDR3 comprising the amino acid sequences of SEQ ID NOs: 1160, 1171, and 1180, respectively, and a LCDR1, a LCDR2, and a LCDR3 comprising the amino acid sequences of SEQ ID NOs: 1148, 1156, and 1157, respectively, according to Kabat.

Embodiment 20. The polypeptide molecule of embodiment [00930] or [00931] wherein the binding affinity is increased by at least 10%, 30%, 50%, 70%, 90%, 1 time, 2 times, 3 times, 4 times, 5 times, 10 times, 50 times, 100 times, 200 times, 300 times, 400 times, 500 times, 600 times, 700 times, 800 times, 900 times, or 1000 times.

Embodiment 21. The polypeptide molecule of any one of embodiments [00913] to [00932], wherein the polypeptide molecule does not bind to TCRβV10-1, TCRβV10-2, or TCRβV10-3.

Embodiment 22. The polypeptide molecule of any one of embodiments [00913] to [00933], wherein the polypeptide molecule comprises an immunoglobulin heavy chain variable (VH) region and/or an immunoglobulin light chain variable (VL) region.

Embodiment 23. The polypeptide molecule of any one of embodiments [00913] to [00934], wherein the polypeptide molecule is a single chain variable fragment (scFv), Fab, CrossFab, Fab′, or F(ab′)2.

Embodiment 24. The polypeptide molecule of any one of embodiments [00913] to [00935], wherein the polypeptide molecule comprises a Fab comprising the VH region, the VL region, a first heavy chain constant region (CH1), and a light chain constant region (CL), wherein the C-terminus of the VH region is connected to the N-terminus of the CH1 region, and the C-terminus of the VL region is connected to the N-terminus of the CL region.

Embodiment 25. The polypeptide molecule of any one of embodiments [00913] to [00935], wherein the polypeptide molecule is a CrossFab comprising the VH region, the VL region, a first heavy chain constant region (CH1), and a light chain constant region (CL), wherein the C-terminus of the VH region is connected to the N-terminus of the CL region, and the C-terminus of the VL region is connected to the N-terminus of the CH1 region.

Embodiment 26. The polypeptide molecule of embodiment [00936] or [00937] wherein the CH1 region and the CL region are connected by a disulfide bond.

Embodiment 27. The polypeptide molecule of any one of embodiments [00936] to [00938], wherein the polypeptide molecule is a charge steering Fab or charge steering CrossFab.

Embodiment 28. The polypeptide molecule of embodiment [00939], wherein the CH1 region of the charge steering Fab or charge steering CrossFab comprises mutations K147E and/or K213E according to EU numbering, and the CL region of the charge steering Fab or charge steering CrossFab comprises mutations E123R and/or Q124K according to EU numbering.

Embodiment 29. The polypeptide molecule of any one of embodiments [00913] to [00935], wherein the polypeptide molecule is a scFv.

Embodiment 30. The polypeptide molecule of embodiment [00941], wherein the C-terminus of the VH region is linked to the N-terminus of the VL region.

Embodiment 31. The polypeptide molecule of embodiment [00941], wherein the C-terminus of the VL region is linked to the N-terminus of the VH region.

Embodiment 32. The polypeptide molecule of any one of embodiments [00941] to [00943], wherein the VH region and the VL region are linked by a peptide linker.

Embodiment 33. The polypeptide molecule of embodiment [00944], wherein the peptide linker comprises the formula of (Gly4Ser)n, wherein n=1-5.

Embodiment 34. The polypeptide molecule of embodiment [00945], wherein the peptide linker is (Gly₄Ser)₃.

Embodiment 35. The polypeptide molecule of embodiment [00945], wherein the peptide linker is (Gly₄Ser)₁.

Embodiment 36. The polypeptide molecule of any one of embodiments [00941] to [00947], wherein the VH region and the VL region is linked by one or more engineered disulfide bond(s).

Embodiment 37. The polypeptide molecule of embodiment [00948], wherein one cysteine that forms the engineered disulfide bond is located in the VH region, and wherein the other cysteine that forms the engineered disulfide bond is located in the VL region.

Embodiment 38. The polypeptide molecule of embodiment [00948] or [00949] wherein the positions of the one or more engineered disulfide bond(s) are selected from the group consisting of: VH44-VL100, VH100B-VL49, VH100-VL50, VH101-VL46, and VH105-VL43, according to EU numbering.

Embodiment 39. The polypeptide molecule of any one of embodiments [00934] to [00950], wherein the VH region comprises an amino acid sequence that is at least about 80%, at least about 85%, at least about 90%, at least about 95%, at least about 97%, at least about 98%, at least about 99%, or 100% identical to any one of SEQ ID NOs: 100, 101, 102, 103, 104, 105, 106, 107, 108, 109, 110, and 111.

Embodiment 40. The polypeptide molecule of any one of embodiments [00934] to [00950], wherein the VL region comprises an amino acid sequence that is at least about 80%, at least about 85%, at least about 90%, at least about 95%, at least about 97%, at least about 98%, at least about 99%, or 100% identical to any one of SEQ ID NOs: 85, 86, 87, 88, 89, 90, 91, 92, and 93.

Embodiment 41. The polypeptide molecule of any one of embodiments [00934] to [00950], wherein:
(i) the VH region comprises an amino acid sequence that is at least about 80%, at least about 85%, at least about 90%, at least about 95%, at least about 97%, at least about 98%, at least about 99%, or 100% identical to SEQ ID NO: 100, and the VL region comprises an amino acid sequence that is at least about 80%, at least about 85%, at least about 90%, at least about 95%, at least about 97%, at least about 98%, at least about 99%, or 100% identical to SEQ ID NO: 85;
(ii) the VH region comprises an amino acid sequence that is at least about 80%, at least about 85%, at least about 90%, at least about 95%, at least about 97%, at least about 98%, at least about 99%, or 100% identical to SEQ ID NO: 101, and the VL region comprises an amino acid sequence that is at least about 80%, at least about 85%, at least about 90%, at least about 95%, at least about 97%, at least about 98%, at least about 99%, or 100% identical to SEQ ID NO: 86;
(iii) the VH region comprises an amino acid sequence that is at least about 80%, at least about 85%, at least about 90%, at least about 95%, at least about 97%, at least about 98%, at least about 99%, or 100% identical to SEQ ID NO: 102, and the VL region comprises an amino acid sequence that is at least about 80%, at least about 85%, at least about 90%, at least about 95%, at least about 97%, at least about 98%, at least about 99%, or 100% identical to SEQ ID NO: 87;
(iv) the VH region comprises an amino acid sequence that is at least about 80%, at least about 85%, at least about 90%, at least about 95%, at least about 97%, at least about 98%, at least about 99%, or 100% identical to SEQ ID NO: 101, and the VL region comprises an amino acid sequence that is at least about 80%, at least about 85%, at least about 90%, at least about 95%, at least about 97%, at least about 98%, at least about 99%, or 100% identical to SEQ ID NO: 85;
(v) the VH region comprises an amino acid sequence that is at least about 80%, at least about 85%, at least about 90%, at least about 95%, at least about 97%, at least about 98%, at least about 99%, or 100% identical to SEQ ID NO: 103, and the VL region comprises an amino acid sequence that is at least about 80%, at least about 85%, at least about 90%, at least about 95%, at least about 97%, at least about 98%, at least about 99%, or 100% identical to SEQ ID NO: 85;
(vi) the VH region comprises an amino acid sequence that is at least about 80%, at least about 85%, at least about 90%, at least about 95%, at least about 97%, at least about 98%, at least about 99%, or 100% identical to SEQ ID NO: 104, and the VL region comprises an amino acid sequence that is at least about 80%, at least about 85%, at least about 90%, at least about 95%, at least about 97%, at least about 98%, at least about 99%, or 100% identical to SEQ ID NO: 88;
(vii) the VH region comprises an amino acid sequence that is at least about 80%, at least about 85%, at least about 90%, at least about 95%, at least about 97%, at least about 98%, at least about 99%, or 100% identical to SEQ ID NO: 105, and the VL region comprises an amino acid sequence that is at least about 80%, at least about 85%, at least about 90%, at least about 95%, at least about 97%, at least about 98%, at least about 99%, or 100% identical to SEQ ID NO: 89;
(viii) the VH region comprises an amino acid sequence that is at least about 80%, at least about 85%, at least about 90%, at least about 95%, at least about 97%, at least about 98%, at least about 99%, or 100% identical to SEQ ID NO: 101, and the VL region comprises an amino acid sequence that is at least about 80%, at least about 85%, at least about 90%, at least about 95%, at least about 97%, at least about 98%, at least about 99%, or 100% identical to SEQ ID NO: 91;
(ix) the VH region comprises an amino acid sequence that is at least about 80%, at least about 85%, at least about 90%, at least about 95%, at least about 97%, at least about 98%, at least about 99%, or 100% identical to SEQ ID NO: 106, and the VL region comprises an amino acid sequence that is at least about 80%, at least about 85%, at least about 90%, at least about 95%, at least about 97%, at least about 98%, at least about 99%, or 100% identical to SEQ ID NO: 87;
(x) the VH region comprises an amino acid sequence that is at least about 80%, at least about 85%, at least about 90%, at least about 95%, at least about 97%, at least about 98%, at least about 99%, or 100% identical to SEQ ID NO: 107, and the VL region comprises an amino acid sequence that is at least about 80%, at least about 85%, at least about 90%, at least about 95%, at least about 97%, at least about 98%, at least about 99%, or 100% identical to SEQ ID NO: 87;
(xi) the VH region comprises an amino acid sequence that is at least about 80%, at least about 85%, at least about 90%, at least about 95%, at least about 97%, at least about 98%, at least about 99%, or 100% identical to SEQ ID NO: 108, and the VL region comprises an amino acid sequence that is at least about 80%, at least about 85%, at least about 90%, at least about 95%, at least about 97%, at least about 98%, at least about 99%, or 100% identical to SEQ ID NO: 89;
(xii) the VH region comprises an amino acid sequence that is at least about 80%, at least about 85%, at least about 90%, at least about 95%, at least about 97%, at least about 98%, at least about 99%, or 100% identical to SEQ ID NO: 109, and the VL region comprises an amino acid sequence that is at least about 80%, at least about 85%, at least about 90%, at least about 95%, at least about 97%, at least about 98%, at least about 99%, or 100% identical to SEQ ID NO: 90;
(xiii) the VH region comprises an amino acid sequence that is at least about 80%, at least about 85%, at least about 90%, at least about 95%, at least about 97%, at least about 98%, at least about 99%, or 100% identical to SEQ ID NO: 109, and the VL region comprises an amino acid sequence that is at least about 80%, at least about 85%, at least about 90%, at least about 95%, at least about 97%, at least about 98%, at least about 99%, or 100% identical to SEQ ID NO: 86;
(xiv) the VH region comprises an amino acid sequence that is at least about 80%, at least about 85%, at least about 90%, at least about 95%, at least about 97%, at least about 98%, at least about 99%, or 100% identical to SEQ ID NO: 106, and the VL region comprises an amino acid sequence that is at least about 80%, at least about 85%, at least about 90%, at least about 95%, at least about 97%, at least about 98%, at least about 99%, or 100% identical to SEQ ID NO: 92;
(xv) the VH region comprises an amino acid sequence that is at least about 80%, at least about 85%, at least about 90%, at least about 95%, at least about 97%, at least about 98%, at least about 99%, or 100% identical to SEQ ID NO: 110, and the VL region comprises an amino acid sequence that is at least about 80%, at least about 85%, at least about 90%, at least about 95%, at least about 97%, at least about 98%, at least about 99%, or 100% identical to SEQ ID NO: 92;
(xvi) the VH region comprises an amino acid sequence that is at least about 80%, at least about 85%, at least about 90%, at least about 95%, at least about 97%, at least about 98%, at least about 99%, or 100% identical to SEQ ID NO: 111, and the VL region comprises an amino acid sequence that is at least about 80%, at least about 85%, at least about 90%, at least about 95%, at least about 97%, at least about 98%, at least about 99%, or 100% identical to SEQ ID NO: 87;
(xvii) the VH region comprises an amino acid sequence that is at least about 80%, at least about 85%, at least about 90%, at least about 95%, at least about 97%, at least about 98%, at least about 99%, or 100% identical to SEQ ID NO: 107, and the VL region comprises an amino acid sequence that is at least about 80%, at least about 85%, at least about 90%, at least about 95%, at least about 97%, at least about 98%, at least about 99%, or 100% identical to SEQ ID NO: 91; or
(xviii) the VH region comprises an amino acid sequence that is at least about 80%, at least about 85%, at least about 90%, at least about 95%, at least about 97%, at least about 98%, at least about 99%, or 100% identical to SEQ ID NO: 111, and the VL region comprises an amino acid sequence that is at least about 80%, at least about 85%, at least about 90%, at least about 95%, at least about 97%, at least about 98%, at least about 99%, or 100% identical to SEQ ID NO: 93.

Embodiment 42. The polypeptide molecule of any one of embodiments [00913] to [00940], and [00951] to [00953], wherein the polypeptide molecule comprises a Fab or CrossFab comprising a light chain and a heavy chain sequence that is at least about 80%, at least about 85%, at least about 90%, at least about 95%, at least about 97%, at least about 98%, at least about 99%, or 100% identical to any one of CLY-010 to CLY-17, and CLY-208-1055.

Embodiment 43. The polypeptide molecule of embodiment 42, wherein the polypeptide molecule comprises a Fab or CrossFab comprising an amino acid sequence that is at least about 80%, at least about 85%, at least about 90%, at least about 95%, at least about 97%, at least about 98%, at least about 99%, or 100% identical to SEQ ID NO: 582; and (ii) a light chain comprising an amino acid sequence that is at least about 80%, at least about 85%, at least about 90%, at least about 95%, at least about 97%, at least about 98%, at least about 99%, or 100% identical to SEQ ID NO: 236.

Embodiment 44. The polypeptide molecule of embodiment 42, wherein the polypeptide molecule comprises a Fab or CrossFab comprising an amino acid sequence that is at least about 80%, at least about 85%, at least about 90%, at least about 95%, at least about 97%, at least about 98%, at least about 99%, or 100% identical to SEQ ID NO: 583; and (ii) a light chain comprising an amino acid sequence that is at least about 80%, at least about 85%, at least about 90%, at least about 95%, at least about 97%, at least about 98%, at least about 99%, or 100% identical to SEQ ID NO: 236.

Embodiment 45. The polypeptide molecule of any one of embodiments [00913] to [00940], and [00951] to [00953], wherein the polypeptide molecule comprises a Fab or CrossFab comprising a light chain and a heavy chain sequence that is at least about 80%, at least about 85%, at least about 90%, at least about 95%, at least about 97%, at least about 98%, at least about 99%, or 100% identical to any one of CLY-1065, CLY-008, and CLY-008-2 to CLY-008-8.

Embodiment 46. The polypeptide molecule of any one of embodiments [00913] to [00935] and [00941] to [00953], wherein the polypeptide molecule comprises an scFv comprising an amino acid sequence that is at least about 80%, at least about 85%, at least about 90%, at least about 95%, at least about 97%, at least about 98%, at least about 99%, or 100% identical to any one of SEQ ID NOs: 187-222.

Embodiment 47. The polypeptide molecule of any one of embodiments [00913] to [00935] and [00941] to [00953], wherein the polypeptide molecule comprises an scFv comprising an amino acid sequence that is at least about 80%, at least about 85%, at least about 90%, at least about 95%, at least about 97%, at least about 98%, at least about 99%, or 100% identical to any one of SEQ ID NOs: 149-186.

Embodiment 48. The polypeptide molecule of any one of embodiments [00934] to [00950], wherein the VH region comprises an amino acid sequence that is at least about 80%, at least about 85%, at least about 90%, at least about 95%, at least about 97%, at least about 98%, at least about 99%, or 100% identical to any one of SEQ ID NOs: 1116-1147.

Embodiment 49. The polypeptide molecule of any one of embodiments [00934] to [00950], wherein the VL region comprises an amino acid sequence that is at least about 80%, at least about 85%, at least about 90%, at least about 95%, at least about 97%, at least about 98%, at least about 99%, or 100% identical to any one of SEQ ID NOs: 1104-1112.

Embodiment 50. The polypeptide molecule of any one of embodiments [00934] to [00950], wherein:
(i) the VH region comprises an amino acid sequence that is at least about 80%, at least about 85%, at least about 90%, at least about 95%, at least about 97%, at least about 98%, at least about 99%, or 100% identical to SEQ ID NO: 1116, and the VL region comprises an amino acid sequence that is at least about 80%, at least about 85%, at least about 90%, at least about 95%, at least about 97%, at least about 98%, at least about 99%, or 100% identical to SEQ ID NO: 1104;
(ii) the VH region comprises an amino acid sequence that is at least about 80%, at least about 85%, at least about 90%, at least about 95%, at least about 97%, at least about 98%, at least about 99%, or 100% identical to SEQ ID NO: 1116, and the VL region comprises an amino acid sequence that is at least about 80%, at least about 85%, at least about 90%, at least about 95%, at least about 97%, at least about 98%, at least about 99%, or 100% identical to SEQ ID NO: 1105;
(iii) the VH region comprises an amino acid sequence that is at least about 80%, at least about 85%, at least about 90%, at least about 95%, at least about 97%, at least about 98%, at least about 99%, or 100% identical to SEQ ID NO: 1117, and the VL region comprises an amino acid sequence that is at least about 80%, at least about 85%, at least about 90%, at least about 95%, at least about 97%, at least about 98%, at least about 99%, or 100% identical to SEQ ID NO: 1104;
(iv) the VH region comprises an amino acid sequence that is at least about 80%, at least about 85%, at least about 90%, at least about 95%, at least about 97%, at least about 98%, at least about 99%, or 100% identical to SEQ ID NO: 1118, and the VL region comprises an amino acid sequence that is at least about 80%, at least about 85%, at least about 90%, at least about 95%, at least about 97%, at least about 98%, at least about 99%, or 100% identical to SEQ ID NO: 1106;
(v) the VH region comprises an amino acid sequence that is at least about 80%, at least about 85%, at least about 90%, at least about 95%, at least about 97%, at least about 98%, at least about 99%, or 100% identical to SEQ ID NO: 1119, and the VL region comprises an amino acid sequence that is at least about 80%, at least about 85%, at least about 90%, at least about 95%, at least about 97%, at least about 98%, at least about 99%, or 100% identical to SEQ ID NO: 1107;
(vi) the VH region comprises an amino acid sequence that is at least about 80%, at least about 85%, at least about 90%, at least about 95%, at least about 97%, at least about 98%, at least about 99%, or 100% identical to SEQ ID NO: 1120, and the VL region comprises an amino acid sequence that is at least about 80%, at least about 85%, at least about 90%, at least about 95%, at least about 97%, at least about 98%, at least about 99%, or 100% identical to SEQ ID NO: 1105;
(vii) the VH region comprises an amino acid sequence that is at least about 80%, at least about 85%, at least about 90%, at least about 95%, at least about 97%, at least about 98%, at least about 99%, or 100% identical to SEQ ID NO: 1121, and the VL region comprises an amino acid sequence that is at least about 80%, at least about 85%, at least about 90%, at least about 95%, at least about 97%, at least about 98%, at least about 99%, or 100% identical to SEQ ID NO: 1108;
(viii) the VH region comprises an amino acid sequence that is at least about 80%, at least about 85%, at least about 90%, at least about 95%, at least about 97%, at least about 98%, at least about 99%, or 100% identical to SEQ ID NO: 1122, and the VL region comprises an amino acid sequence that is at least about 80%, at least about 85%, at least about 90%, at least about 95%, at least about 97%, at least about 98%, at least about 99%, or 100% identical to SEQ ID NO: 1109;
(ix) the VH region comprises an amino acid sequence that is at least about 80%, at least about 85%, at least about 90%, at least about 95%, at least about 97%, at least about 98%, at least about 99%, or 100% identical to SEQ ID NO: 1123, and the VL region comprises an amino acid sequence that is at least about 80%, at least about 85%, at least about 90%, at least about 95%, at least about 97%, at least about 98%, at least about 99%, or 100% identical to SEQ ID NO: 1110;
(x) the VH region comprises an amino acid sequence that is at least about 80%, at least about 85%, at least about 90%, at least about 95%, at least about 97%, at least about 98%, at least about 99%, or 100% identical to SEQ ID NO: 1124, and the VL region comprises an amino acid sequence that is at least about 80%, at least about 85%, at least about 90%, at least about 95%, at least about 97%, at least about 98%, at least about 99%, or 100% identical to SEQ ID NO: 1105;
(xi) the VH region comprises an amino acid sequence that is at least about 80%, at least about 85%, at least about 90%, at least about 95%, at least about 97%, at least about 98%, at least about 99%, or 100% identical to SEQ ID NO: 1125, and the VL region comprises an amino acid sequence that is at least about 80%, at least about 85%, at least about 90%, at least about 95%, at least about 97%, at least about 98%, at least about 99%, or 100% identical to SEQ ID NO: 1105;
(xii) the VH region comprises an amino acid sequence that is at least about 80%, at least about 85%, at least about 90%, at least about 95%, at least about 97%, at least about 98%, at least about 99%, or 100% identical to SEQ ID NO: 1126, and the VL region comprises an amino acid sequence that is at least about 80%, at least about 85%, at least about 90%, at least about 95%, at least about 97%, at least about 98%, at least about 99%, or 100% identical to SEQ ID NO: 1111;
(xiii) the VH region comprises an amino acid sequence that is at least about 80%, at least about 85%, at least about 90%, at least about 95%, at least about 97%, at least about 98%, at least about 99%, or 100% identical to SEQ ID NO: 1127, and the VL region comprises an amino acid sequence that is at least about 80%, at least about 85%, at least about 90%, at least about 95%, at least about 97%, at least about 98%, at least about 99%, or 100% identical to SEQ ID NO: 1108;
(xiv) the VH region comprises an amino acid sequence that is at least about 80%, at least about 85%, at least about 90%, at least about 95%, at least about 97%, at least about 98%, at least about 99%, or 100% identical to SEQ ID NO: 1128, and the VL region comprises an amino acid sequence that is at least about 80%, at least about 85%, at least about 90%, at least about 95%, at least about 97%, at least about 98%, at least about 99%, or 100% identical to SEQ ID NO: 1109;
(xv) the VH region comprises an amino acid sequence that is at least about 80%, at least about 85%, at least about 90%, at least about 95%, at least about 97%, at least about 98%, at least about 99%, or 100% identical to SEQ ID NO: 1129, and the VL region comprises an amino acid sequence that is at least about 80%, at least about 85%, at least about 90%, at least about 95%, at least about 97%, at least about 98%, at least about 99%, or 100% identical to SEQ ID NO: 1111;
(xvi) the VH region comprises an amino acid sequence that is at least about 80%, at least about 85%, at least about 90%, at least about 95%, at least about 97%, at least about 98%, at least about 99%, or 100% identical to SEQ ID NO: 1130, and the VL region comprises an amino acid sequence that is at least about 80%, at least about 85%, at least about 90%, at least about 95%, at least about 97%, at least about 98%, at least about 99%, or 100% identical to SEQ ID NO: 1107;
(xvii) the VH region comprises an amino acid sequence that is at least about 80%, at least about 85%, at least about 90%, at least about 95%, at least about 97%, at least about 98%, at least about 99%, or 100% identical to SEQ ID NO: 1131, and the VL region comprises an amino acid sequence that is at least about 80%, at least about 85%, at least about 90%, at least about 95%, at least about 97%, at least about 98%, at least about 99%, or 100% identical to SEQ ID NO: 1109;
(xviii) the VH region comprises an amino acid sequence that is at least about 80%, at least about 85%, at least about 90%, at least about 95%, at least about 97%, at least about 98%, at least about 99%, or 100% identical to SEQ ID NO: 1132, and the VL region comprises an amino acid sequence that is at least about 80%, at least about 85%, at least about 90%, at least about 95%, at least about 97%, at least about 98%, at least about 99%, or 100% identical to SEQ ID NO: 1111;
(xix) the VH region comprises an amino acid sequence that is at least about 80%, at least about 85%, at least about 90%, at least about 95%, at least about 97%, at least about 98%, at least about 99%, or 100% identical to SEQ ID NO: 1116, and the VL region comprises an amino acid sequence that is at least about 80%, at least about 85%, at least about 90%, at least about 95%, at least about 97%, at least about 98%, at least about 99%, or 100% identical to SEQ ID NO: 1108;
(xx) the VH region comprises an amino acid sequence that is at least about 80%, at least about 85%, at least about 90%, at least about 95%, at least about 97%, at least about 98%, at least about 99%, or 100% identical to SEQ ID NO: 1133, and the VL region comprises an amino acid sequence that is at least about 80%, at least about 85%, at least about 90%, at least about 95%, at least about 97%, at least about 98%, at least about 99%, or 100% identical to SEQ ID NO: 1111;
(xxi) the VH region comprises an amino acid sequence that is at least about 80%, at least about 85%, at least about 90%, at least about 95%, at least about 97%, at least about 98%, at least about 99%, or 100% identical to SEQ ID NO: 1134, and the VL region comprises an amino acid sequence that is at least about 80%, at least about 85%, at least about 90%, at least about 95%, at least about 97%, at least about 98%, at least about 99%, or 100% identical to SEQ ID NO: 1106;
(xxii) the VH region comprises an amino acid sequence that is at least about 80%, at least about 85%, at least about 90%, at least about 95%, at least about 97%, at least about 98%, at least about 99%, or 100% identical to SEQ ID NO: 1135, and the VL region comprises an amino acid sequence that is at least about 80%, at least about 85%, at least about 90%, at least about 95%, at least about 97%, at least about 98%, at least about 99%, or 100% identical to SEQ ID NO: 1108;
(xxiii) the VH region comprises an amino acid sequence that is at least about 80%, at least about 85%, at least about 90%, at least about 95%, at least about 97%, at least about 98%, at least about 99%, or 100% identical to SEQ ID NO: 1136, and the VL region comprises an amino acid sequence that is at least about 80%, at least about 85%, at least about 90%, at least about 95%, at least about 97%, at least about 98%, at least about 99%, or 100% identical to SEQ ID NO: 1112;
(xxiv) the VH region comprises an amino acid sequence that is at least about 80%, at least about 85%, at least about 90%, at least about 95%, at least about 97%, at least about 98%, at least about 99%, or 100% identical to SEQ ID NO: 1137, and the VL region comprises an amino acid sequence that is at least about 80%, at least about 85%, at least about 90%, at least about 95%, at least about 97%, at least about 98%, at least about 99%, or 100% identical to SEQ ID NO: 1110;
(xxv) the VH region comprises an amino acid sequence that is at least about 80%, at least about 85%, at least about 90%, at least about 95%, at least about 97%, at least about 98%, at least about 99%, or 100% identical to SEQ ID NO: 1127, and the VL region comprises an amino acid sequence that is at least about 80%, at least about 85%, at least about 90%, at least about 95%, at least about 97%, at least about 98%, at least about 99%, or 100% identical to SEQ ID NO: 1104;
(xxvi) the VH region comprises an amino acid sequence that is at least about 80%, at least about 85%, at least about 90%, at least about 95%, at least about 97%, at least about 98%, at least about 99%, or 100% identical to SEQ ID NO: 1138, and the VL region comprises an amino acid sequence that is at least about 80%, at least about 85%, at least about 90%, at least about 95%, at least about 97%, at least about 98%, at least about 99%, or 100% identical to SEQ ID NO: 1106;
(xxvii) the VH region comprises an amino acid sequence that is at least about 80%, at least about 85%, at least about 90%, at least about 95%, at least about 97%, at least about 98%, at least about 99%, or 100% identical to SEQ ID NO: 1139, and the VL region comprises an amino acid sequence that is at least about 80%, at least about 85%, at least about 90%, at least about 95%, at least about 97%, at least about 98%, at least about 99%, or 100% identical to SEQ ID NO: 1105;
(xxviii) the VH region comprises an amino acid sequence that is at least about 80%, at least about 85%, at least about 90%, at least about 95%, at least about 97%, at least about 98%, at least about 99%, or 100% identical to SEQ ID NO: 1132, and the VL region comprises an amino acid sequence that is at least about 80%, at least about 85%, at least about 90%, at least about 95%, at least about 97%, at least about 98%, at least about 99%, or 100% identical to SEQ ID NO: 1109;
(xxix) the VH region comprises an amino acid sequence that is at least about 80%, at least about 85%, at least about 90%, at least about 95%, at least about 97%, at least about 98%, at least about 99%, or 100% identical to SEQ ID NO: 1140, and the VL region comprises an amino acid sequence that is at least about 80%, at least about 85%, at least about 90%, at least about 95%, at least about 97%, at least about 98%, at least about 99%, or 100% identical to SEQ ID NO: 1109;
(xxx) the VH region comprises an amino acid sequence that is at least about 80%, at least about 85%, at least about 90%, at least about 95%, at least about 97%, at least about 98%, at least about 99%, or 100% identical to SEQ ID NO: 1141, and the VL region comprises an amino acid sequence that is at least about 80%, at least about 85%, at least about 90%, at least about 95%, at least about 97%, at least about 98%, at least about 99%, or 100% identical to SEQ ID NO: 1104;
(xxxi) the VH region comprises an amino acid sequence that is at least about 80%, at least about 85%, at least about 90%, at least about 95%, at least about 97%, at least about 98%, at least about 99%, or 100% identical to SEQ ID NO: 1142, and the VL region comprises an amino acid sequence that is at least about 80%, at least about 85%, at least about 90%, at least about 95%, at least about 97%, at least about 98%, at least about 99%, or 100% identical to SEQ ID NO: 1104;
(xxxii) the VH region comprises an amino acid sequence that is at least about 80%, at least about 85%, at least about 90%, at least about 95%, at least about 97%, at least about 98%, at least about 99%, or 100% identical to SEQ ID NO: 1143, and the VL region comprises an amino acid sequence that is at least about 80%, at least about 85%, at least about 90%, at least about 95%, at least about 97%, at least about 98%, at least about 99%, or 100% identical to SEQ ID NO: 1104;
(xxxiii) the VH region comprises an amino acid sequence that is at least about 80%, at least about 85%, at least about 90%, at least about 95%, at least about 97%, at least about 98%, at least about 99%, or 100% identical to SEQ ID NO: 1144, and the VL region comprises an amino acid sequence that is at least about 80%, at least about 85%, at least about 90%, at least about 95%, at least about 97%, at least about 98%, at least about 99%, or 100% identical to SEQ ID NO: 1111;
(xxxiv) the VH region comprises an amino acid sequence that is at least about 80%, at least about 85%, at least about 90%, at least about 95%, at least about 97%, at least about 98%, at least about 99%, or 100% identical to SEQ ID NO: 1145, and the VL region comprises an amino acid sequence that is at least about 80%, at least about 85%, at least about 90%, at least about 95%, at least about 97%, at least about 98%, at least about 99%, or 100% identical to SEQ ID NO: 1104;
(xxxv) the VH region comprises an amino acid sequence that is at least about 80%, at least about 85%, at least about 90%, at least about 95%, at least about 97%, at least about 98%, at least about 99%, or 100% identical to SEQ ID NO: 1117, and the VL region comprises an amino acid sequence that is at least about 80%, at least about 85%, at least about 90%, at least about 95%, at least about 97%, at least about 98%, at least about 99%, or 100% identical to SEQ ID NO: 1106;
(xxxvi) the VH region comprises an amino acid sequence that is at least about 80%, at least about 85%, at least about 90%, at least about 95%, at least about 97%, at least about 98%, at least about 99%, or 100% identical to SEQ ID NO: 1146, and the VL region comprises an amino acid sequence that is at least about 80%, at least about 85%, at least about 90%, at least about 95%, at least about 97%, at least about 98%, at least about 99%, or 100% identical to SEQ ID NO: 1104; or
(xxxvii) the VH region comprises an amino acid sequence that is at least about 80%, at least about 85%, at least about 90%, at least about 95%, at least about 97%, at least about 98%, at least about 99%, or 100% identical to SEQ ID NO: 1147, and the VL region comprises an amino acid sequence that is at least about 80%, at least about 85%, at least about 90%, at least about 95%, at least about 97%, at least about 98%, at least about 99%, or 100% identical to SEQ ID NO: 1110.

Embodiment 51. The polypeptide molecule of any one of embodiments [00913] to [00940], and [00960] to [00962], wherein the polypeptide molecule comprises a Fab or CrossFab comprising a light chain and a heavy chain sequence that is at least about 80%, at least about 85%, at least about 90%, at least about 95%, at least about 97%, at least about 98%, at least about 99%, or 100% identical to any one of CLY-035, CLY-035-2 to CLY-035-9, CLY-036, CLY-036-2 to CLY-036-6, and CLY-037 to CLY-058.

Embodiment 52. The polypeptide molecule of any one of embodiments [00913] to [00940], and [00960] to [00962], wherein the polypeptide molecule comprises a Fab or CrossFab comprising a light chain and a heavy chain sequence that is at least about 80%, at least about 85%, at least about 90%, at least about 95%, at least about 97%, at least about 98%, at least about 99%, or 100% identical to any one of CLY-034 and CLY-034-2 to CLY-034-6.

Embodiment 53. The polypeptide molecule of any one of embodiments [00913] to [00935], [00941] to [00950], and [00960] to [00962], wherein the polypeptide molecule comprises an scFv comprising an amino acid sequence that is at least about 80%, at least about 85%, at least about 90%, at least about 95%, at least about 97%, at least about 98%, at least about 99%, or 100% identical to any one of SEQ ID NOs: 1204-1277.

Embodiment 54. The polypeptide molecule of any one of embodiments [00913] to [00935], [00941] to [00950], and [00960] to [00962], wherein the polypeptide molecule comprises an scFv comprising an amino acid sequence that is at least about 80%, at least about 85%, at least about 90%, at least about 95%, at least about 97%, at least about 98%, at least about 99%, or 100% identical to SEQ ID NO: 1203.

Embodiment 55. A multi-specific polypeptide molecule comprising:
(i) a first binding domain that is the polypeptide molecule of any one of embodiments [00913] to [00965], or a functional fragment thereof; and
(ii) a second binding domain.

Embodiment 56. The multi-specific polypeptide molecule of embodiment [00967] wherein the second binding domain specifically binds to a protein expressed on a tumor cell or a B cell.

Embodiment 57. The multi-specific polypeptide molecule of embodiment [00968] wherein the protein expressed on the tumor cell is a tumor associated antigen.

Embodiment 58. The multi-specific polypeptide molecule of embodiment [00968] wherein the protein expressed on the B cell is CD19.

Embodiment 59. The multi-specific polypeptide molecule of any one of embodiments [00967] to [00970], wherein the second binding domain comprises:
(i) an immunoglobulin heavy chain variable (VH) region comprising a heavy chain CDR1 (HCDR1), a heavy chain CDR2 (HCDR2), and a heavy chain CDR3 (HCDR3); and/or
(ii) an immunoglobulin light chain variable (VL) region comprising a light chain CDR1 (LCDR1), a light chain CDR2 (LCDR2), and a light chain CDR3 (LCDR3).

Embodiment 60. The multi-specific polypeptide molecule of any one of embodiments [00967] to [00971], wherein the second binding domain comprises: the HCDR1, the HCDR2, and the HCDR3 comprising the amino acid sequences of SEQ ID NOs: 1353, 1355, and 1357, respectively, and the LCDR1, the LCDR2, and the LCDR3 comprising the amino acid sequences of SEQ ID NOs: 1347, 1349, and 1351, respectively.

Embodiment 61. The multi-specific polypeptide molecule of any one of embodiments [00967] to [00971], wherein the second binding domain comprises: the HCDR1, the HCDR2, and the HCDR3 comprising the amino acid sequences of SEQ ID NOs: 1354, 1356, and 1358, respectively, and the LCDR1, the LCDR2, and the LCDR3 comprising the amino acid sequences of SEQ ID NOs: 1348, 1350, and 1352, respectively; and wherein the CDRs are numbered with reference to the Kabat numbering scheme.

Embodiment 62. The multi-specific polypeptide molecule of any one of embodiments [00967] to 0, wherein the second binding domain is a single chain variable fragment (scFv), Fab, CrossFab, Fab′, or F(ab′)2.

Embodiment 63. The multi-specific polypeptide molecule of any one of embodiments [00967] to [00974], wherein the second binding domain comprises a Fab comprising the VH region, the VL region, a first heavy chain constant region (CH1), and a light chain constant region (CL), wherein the C-terminus of the VH region is connected to the N-terminus of the CH1 region, and the C-terminus of the VL region is connected to the N-terminus of the CL region.

Embodiment 64. The multi-specific polypeptide molecule of any one of embodiments [00967] to [00974], wherein the second binding domain is a CrossFab comprising the VH region, the VL region, a first heavy chain constant region (CH1), and a light chain constant region (CL), wherein the C-terminus of the VH region is connected to the N-terminus of the CL region, and the C-terminus of the VL region is connected to the N-terminus of the CH1 region.

Embodiment 65. The multi-specific polypeptide molecule of embodiment [00975] or [00976], wherein the CH1 region and the CL region are connected by a disulfide bond.

Embodiment 66. The multi-specific polypeptide molecule of any one of embodiments [00975] to [00977], wherein the second binding domain is a charge steering Fab or charge steering CrossFab.

Embodiment 67. The multi-specific polypeptide molecule of embodiment [00978] wherein the CH1 region of the charge steering Fab or charge steering CrossFab comprises mutations K147E and/or K213E according to EU numbering, and the CL region of the charge steering Fab or charge steering CrossFab comprises mutations E123R and/or Q124K according to EU numbering.

Embodiment 68. The multi-specific polypeptide molecule of any one of embodiments [00967] to [00974], wherein the second binding domain is a scFv.

Embodiment 69. The multi-specific polypeptide molecule of embodiment [00980] wherein the C-terminus of the VH region is linked to the N-terminus of the VL region.

Embodiment 70. The multi-specific polypeptide molecule of embodiment [00980] wherein the C-terminus of the VL region is linked to the N-terminus of the VH region.

Embodiment 71. The multi-specific polypeptide molecule of any one of embodiments [00980] to [00982], wherein the VH region and the VL region are linked by a peptide linker.

Embodiment 72. The multi-specific polypeptide molecule of embodiment [00983] wherein the peptide linker comprises the formula of (Gly₄Ser)ₙ, wherein n=1-5.

Embodiment 73. The multi-specific polypeptide molecule of embodiment [00984] wherein the peptide linker is (Gly₄Ser)₃.

Embodiment 74. The multi-specific polypeptide molecule of embodiment [00984] wherein the peptide linker is (Gly₄Ser)₁.

Embodiment 75. The multi-specific polypeptide molecule of any one of embodiments [00967] to [00986], wherein the VH region of the second binding domain comprises an amino acid sequence that is at least about 80%, at least about 85%, at least about 90%, at least about 95%, at least about 97%, at least about 98%, at least about 99%, or 100% identical to any one of SEQ ID NOs: 1345, 1346, 1370, and 1371.

Embodiment 76. The multi-specific polypeptide molecule of any one of embodiments [00967] to [00986], wherein the VL region of the second binding domain comprises an amino acid sequence that is at least about 80%, at least about 85%, at least about 90%, at least about 95%, at least about 97%, at least about 98%, at least about 99%, or 100% identical to any one of SEQ ID NOs: 1343, 1344, 1368, and 1369.

Embodiment 77. The multi-specific polypeptide molecule of any one of embodiments [00967] to [00986], wherein:
(i) the VH region of the second binding domain comprises an amino acid sequence that is at least about 80%, at least about 85%, at least about 90%, at least about 95%, at least about 97%, at least about 98%, at least about 99%, or 100% identical to SEQ ID NO: 1345, and the VL region of the second binding domain comprises an amino acid sequence that is at least about 80%, at least about 85%, at least about 90%, at least about 95%, at least about 97%, at least about 98%, at least about 99%, or 100% identical to SEQ ID NO: 1343; or
(ii) the VH region of the second binding domain comprises an amino acid sequence that is at least about 80%, at least about 85%, at least about 90%, at least about 95%, at least about 97%, at least about 98%, at least about 99%, or 100% identical to SEQ ID NO: 1346, and the VL region of the second binding domain comprises an amino acid sequence that is at least about 80%, at least about 85%, at least about 90%, at least about 95%, at least about 97%, at least about 98%, at least about 99%, or 100% identical to SEQ ID NO: 1344.

Embodiment 78. The multi-specific polypeptide molecule of any one of embodiments [00967] to [00979], and [00987] to [00989], wherein the second binding domain comprises a Fab or CrossFab comprising (i) a heavy chain comprising an amino acid sequence that is at least about 80%, at least about 85%, at least about 90%, at least about 95%, at least about 97%, at least about 98%, at least about 99%, or 100% identical to SEQ ID NO: 1370 or 1371; and (ii) a light chain comprising an amino acid sequence that is at least about 80%, at least about 85%, at least about 90%, at least about 95%, at least about 97%, at least about 98%, at least about 99%, or 100% identical to SEQ ID NO: 1368 or 1369.

Embodiment 79. The multi-specific polypeptide molecule of any one of embodiments [00967] to [00974], and [00980] to [00989], wherein the second binding domain comprises an scFv comprising an amino acid sequence that is at least about 80%, at least about 85%, at least about 90%, at least about 95%, at least about 97%, at least about 98%, at least about 99%, or 100% identical to SEQ ID NO: 1367.

Embodiment 80. The multi-specific polypeptide molecule of any one of embodiments [00967] to 79, wherein the first binding domain is an scFv, and wherein the second binding domain comprises a Fab.

Embodiment 81. The multi-specific polypeptide molecule of any one of embodiments [00967] to 79, wherein the first binding domain is an scFv, and wherein the second binding domain is a CrossFab

Embodiment 82. The multi-specific polypeptide molecule of any one of embodiments [00967] to 79, wherein the first binding domain is an scFv, a charge steering Fab, or a charge steering CrossFab.

Embodiment 83. The multi-specific polypeptide molecule of any one of embodiments [00967] to 79, wherein the first binding domain comprises a Fab and wherein the second binding domain is an scFv.

Embodiment 84. The multi-specific polypeptide molecule of any one of embodiments [00967] to 79, wherein the first binding domain is a CrossFab and wherein the second binding domain is an scFv.

Embodiment 85. The multi-specific polypeptide molecule of any one of embodiments [00967] to [00996], wherein the first binding domain is a charge steering Fab or a charge steering CrossFab, and wherein the second binding domain is an scFv.

Embodiment 86. The multi-specific polypeptide molecule of any one of embodiments [00967] to [00996], wherein the first binding domain is an scFv, and wherein the second binding domain is an scFv.

Embodiment 87. The multi-specific polypeptide molecule of any one of embodiments [00967] to Error! Reference source not found., wherein the first binding domain and the second binding domain are linked by a peptide linker.

Embodiment 88. The multi-specific polypeptide molecule of embodiment [00999] wherein the peptide linker comprises the formula of (Gly₄Ser)ₙ, wherein n=1-5.

Embodiment 89. The multi-specific polypeptide molecule of embodiment [001000] wherein the peptide linker is (Gly₄Ser)₃.

Embodiment 90. The multi-specific polypeptide molecule of embodiment [001000] wherein the peptide linker is (Gly₄Ser)₁.

Embodiment 91. The multi-specific polypeptide molecule of any one of embodiments [00967] to [001002], wherein the multi-specific polypeptide molecule further comprises a half-life extension moiety.

Embodiment 92. The multi-specific polypeptide molecule of embodiment [001003] wherein the half-life extension moiety is selected from the group consisting of a fragment crystallizable (Fc) region (e.g., a single chain Fc), albumin (e.g., human serum albumin), transferrin, a binding domain that specifically binds to transferrin or albumin (e.g., an albumin binding domain (ABD)), an unstructured biodegradable proteins (XTEN), a polyethylene glycol (PEG), a glycine-rich homo-amino-acid polymer (HAP), a hydroxyethyl starch (HES), a polysialic acid (PSA), an immunoglobulin binding polypeptide, and a conformationally disordered polypeptide comprising Proline, Alanine and Serine (PAS), or is a derivative or functional fragment thereof.

Embodiment 93. The multi-specific polypeptide molecule of embodiment [001003] or [001004], wherein the half-life extension moiety is a fragment crystallizable (Fc) region.

Embodiment 94. The multi-specific polypeptide molecule of embodiment [001005] wherein the Fc region comprises two polypeptides.

Embodiment 95. The multi-specific polypeptide molecule of embodiment [001006], wherein the first binding domain is linked to one of the polypeptides of the Fc region and the second binding domain is linked to the other polypeptide of the Fc region.

Embodiment 96. The multi-specific polypeptide molecule of embodiment [001006] wherein the first binding domain and the second binding domain are linked to the same polypeptides of the Fc region.

Embodiment 97. The multi-specific polypeptide molecule of any one of embodiments [001006] to [001008], wherein the Fc region has a knobs-into-holes format, wherein the first polypeptide of the Fc region comprises one or more hole mutations, and wherein second polypeptide of the Fc region comprises one or more knob mutations.

Embodiment 98. The multi-specific polypeptide molecule of embodiment [001009] wherein the first polypeptide of the Fc region comprises mutations T366S, L368A and Y407V according to EU numbering, and wherein the second polypeptide of the Fc region comprises a mutation T366W according to EU numbering.

Embodiment 99. The multi-specific polypeptide molecule of embodiment [001009] or [001010], wherein the first polypeptide of the Fc region comprising the one or more hole mutations further comprises a mutation Y349C according to EU numbering, wherein the second polypeptide of the Fc region comprising the one or more knob mutations further comprises a mutation S354C according to EU numbering, and wherein the Fc region comprises an engineered disulfide bond formed between these two cysteines.

Embodiment 100. The multi-specific polypeptide molecule of any one of embodiments [001006] to [001011], wherein one or both of the polypeptides of the Fc region comprise mutations L234A, L235A, and P329G according to EU numbering.

Embodiment 101. The multi-specific polypeptide molecule of any one of embodiments [001006] to [001012], wherein one or both of the polypeptides of the Fc region comprise the mutation N297G according to EU numbering.

Embodiment 102. The multi-specific polypeptide molecule of any one of embodiments [001006] to [001013], wherein one or both of the polypeptides of the Fc region comprise mutations H435R and Y436F according to EU numbering.

Embodiment 103. The multi-specific polypeptide molecule of any one of embodiments [001005] to [001014], wherein the Fc region lacks one or more effector functions and/or lacks glycosylation.

Embodiment 104. The multi-specific polypeptide molecule of embodiment [001015], wherein the effector function is selected from the group consisting of antibody-dependent cellular cytotoxicity (ADCC), antibody-dependent cellular phagocytosis (ADCP), complement-dependent cytotoxicity (CDC), and binding to a Fcγ receptor (FcγR), or is any combination thereof.

Embodiment 105. The multi-specific polypeptide molecule of any one of embodiments [001005] to [001016], wherein the Fc region lacks glycine-lysine (GK) residues at the C terminus.

Embodiment 106. The multi-specific polypeptide molecule of any one of embodiments [001005] to [001016], wherein the first polypeptide of the Fc region comprises an amino acid sequence that is at least about 80%, at least about 85%, at least about 90%, at least about 95%, at least about 97%, at least about 98%, at least about 99%, or 100% identical to SEQ ID NO: 1412, and wherein the second polypeptide of the Fc region comprises an amino acid sequence that is at least about 80%, at least about 85%, at least about 90%, at least about 95%, at least about 97%, at least about 98%, at least about 99%, or 100% identical to SEQ ID NO: 1414.

Embodiment 107. The multi-specific polypeptide molecule of any one of embodiments [001005] to [001016], wherein the first polypeptide of the Fc region comprises an amino acid sequence that is at least about 80%, at least about 85%, at least about 90%, at least about 95%, at least about 97%, at least about 98%, at least about 99%, or 100% identical to SEQ ID NO: 1413, and wherein the second polypeptide of the Fc region comprises an amino acid sequence that is at least about 80%, at least about 85%, at least about 90%, at least about 95%, at least about 97%, at least about 98%, at least about 99%, or 100% identical to SEQ ID NO: 1415.

Embodiment 108. The multi-specific polypeptide molecule of any one of embodiments [00967] to 107, wherein the multi-specific polypeptide molecule comprises three polypeptides.

Embodiment 109. The multi-specific polypeptide molecule of embodiment [001020] wherein:
(i) the first polypeptide comprises an amino acid sequence that is at least about 80%, at least about 85%, at least about 90%, at least about 95%, at least about 97%, at least about 98%, at least about 99%, or 100% identical to SEQ ID NO: 1412, the second polypeptide comprises an amino acid sequence that is at least about 80%, at least about 85%, at least about 90%, at least about 95%, at least about 97%, at least about 98%, at least about 99%, or 100% identical to SEQ ID NO: 1416, and the third polypeptide comprises an amino acid sequence that is at least about 80%, at least about 85%, at least about 90%, at least about 95%, at least about 97%, at least about 98%, at least about 99%, or 100% identical to SEQ ID NO: 1417;
(ii) the first polypeptide comprises an amino acid sequence that is at least about 80%, at least about 85%, at least about 90%, at least about 95%, at least about 97%, at least about 98%, at least about 99%, or 100% identical to SEQ ID NO: 1412, the second polypeptide comprises an amino acid sequence that is at least about 80%, at least about 85%, at least about 90%, at least about 95%, at least about 97%, at least about 98%, at least about 99%, or 100% identical to SEQ ID NO: 1369, and the third polypeptide comprises an amino acid sequence that is at least about 80%, at least about 85%, at least about 90%, at least about 95%, at least about 97%, at least about 98%, at least about 99%, or 100% identical to SEQ ID NO: 1418;
(iii) the first polypeptide comprises an amino acid sequence that is at least about 80%, at least about 85%, at least about 90%, at least about 95%, at least about 97%, at least about 98%, at least about 99%, or 100% identical to SEQ ID NO: 1412, the second polypeptide comprises an amino acid sequence that is at least about 80%, at least about 85%, at least about 90%, at least about 95%, at least about 97%, at least about 98%, at least about 99%, or 100% identical to SEQ ID NO: 1419, and the third polypeptide comprises an amino acid sequence that is at least about 80%, at least about 85%, at least about 90%, at least about 95%, at least about 97%, at least about 98%, at least about 99%, or 100% identical to SEQ ID NO: 1417;
(iv) the first polypeptide comprises an amino acid sequence that is at least about 80%, at least about 85%, at least about 90%, at least about 95%, at least about 97%, at least about 98%, at least about 99%, or 100% identical to SEQ ID NO: 1412, the second polypeptide comprises an amino acid sequence that is at least about 80%, at least about 85%, at least about 90%, at least about 95%, at least about 97%, at least about 98%, at least about 99%, or 100% identical to SEQ ID NO: 1420, and the third polypeptide comprises an amino acid sequence that is at least about 80%, at least about 85%, at least about 90%, at least about 95%, at least about 97%, at least about 98%, at least about 99%, or 100% identical to SEQ ID NO: 1417;
(v) the first polypeptide comprises an amino acid sequence that is at least about 80%, at least about 85%, at least about 90%, at least about 95%, at least about 97%, at least about 98%, at least about 99%, or 100% identical to SEQ ID NO: 1412, the second polypeptide comprises an amino acid sequence that is at least about 80%, at least about 85%, at least about 90%, at least about 95%, at least about 97%, at least about 98%, at least about 99%, or 100% identical to SEQ ID NO: 1369, and the third polypeptide comprises an amino acid sequence that is at least about 80%, at least about 85%, at least about 90%, at least about 95%, at least about 97%, at least about 98%, at least about 99%, or 100% identical to SEQ ID NO: 1421;
(vi) the first polypeptide comprises an amino acid sequence that is at least about 80%, at least about 85%, at least about 90%, at least about 95%, at least about 97%, at least about 98%, at least about 99%, or 100% identical to SEQ ID NO: 1412, the second polypeptide comprises an amino acid sequence that is at least about 80%, at least about 85%, at least about 90%, at least about 95%, at least about 97%, at least about 98%, at least about 99%, or 100% identical to SEQ ID NO: 1369, and the third polypeptide comprises an amino acid sequence that is at least about 80%, at least about 85%, at least about 90%, at least about 95%, at least about 97%, at least about 98%, at least about 99%, or 100% identical to SEQ ID NO: 1422; or
(vii) the first polypeptide comprises an amino acid sequence that is at least about 80%, at least about 85%, at least about 90%, at least about 95%, at least about 97%, at least about 98%, at least about 99%, or 100% identical to SEQ ID NO: 1412, the second polypeptide comprises an amino acid sequence that is at least about 80%, at least about 85%, at least about 90%, at least about 95%, at least about 97%, at least about 98%, at least about 99%, or 100% identical to SEQ ID NO: 1369, and the third polypeptide comprises an amino acid sequence that is at least about 80%, at least about 85%, at least about 90%, at least about 95%, at least about 97%, at least about 98%, at least about 99%, or 100% identical to SEQ ID NO: 1423.

Embodiment 110. The multi-specific polypeptide molecule of embodiment [001020] wherein:
(i) the first polypeptide comprises an amino acid sequence that is at least about 80%, at least about 85%, at least about 90%, at least about 95%, at least about 97%, at least about 98%, at least about 99%, or 100% identical to SEQ ID NO: 1412, the second polypeptide comprises an amino acid sequence that is at least about 80%, at least about 85%, at least about 90%, at least about 95%, at least about 97%, at least about 98%, at least about 99%, or 100% identical to SEQ ID NO: 1419, and the third polypeptide comprises an amino acid sequence that is at least about 80%, at least about 85%, at least about 90%, at least about 95%, at least about 97%, at least about 98%, at least about 99%, or 100% identical to SEQ ID NO: 1417;
(ii) the first polypeptide comprises an amino acid sequence that is at least about 80%, at least about 85%, at least about 90%, at least about 95%, at least about 97%, at least about 98%, at least about 99%, or 100% identical to SEQ ID NO: 1412, the second polypeptide comprises an amino acid sequence that is at least about 80%, at least about 85%, at least about 90%, at least about 95%, at least about 97%, at least about 98%, at least about 99%, or 100% identical to SEQ ID NO: 1420, and the third polypeptide comprises an amino acid sequence that is at least about 80%, at least about 85%, at least about 90%, at least about 95%, at least about 97%, at least about 98%, at least about 99%, or 100% identical to SEQ ID NO: 1417;
(iii) the first polypeptide comprises an amino acid sequence that is at least about 80%, at least about 85%, at least about 90%, at least about 95%, at least about 97%, at least about 98%, at least about 99%, or 100% identical to SEQ ID NO: 1412, the second polypeptide comprises an amino acid sequence that is at least about 80%, at least about 85%, at least about 90%, at least about 95%, at least about 97%, at least about 98%, at least about 99%, or 100% identical to SEQ ID NO: 1369, and the third polypeptide comprises an amino acid sequence that is at least about 80%, at least about 85%, at least about 90%, at least about 95%, at least about 97%, at least about 98%, at least about 99%, or 100% identical to SEQ ID NO: 1421; or
(iv) the first polypeptide comprises an amino acid sequence that is at least about 80%, at least about 85%, at least about 90%, at least about 95%, at least about 97%, at least about 98%, at least about 99%, or 100% identical to SEQ ID NO: 1412, the second polypeptide comprises an amino acid sequence that is at least about 80%, at least about 85%, at least about 90%, at least about 95%, at least about 97%, at least about 98%, at least about 99%, or 100% identical to SEQ ID NO: 1369, and the third polypeptide comprises an amino acid sequence that is at least about 80%, at least about 85%, at least about 90%, at least about 95%, at least about 97%, at least about 98%, at least about 99%, or 100% identical to SEQ ID NO: 1422.

Embodiment 111. A multi-specific polypeptide molecule comprising:
(i) a first binding domain that specifically binds to T cell receptor beta variable region (TCRβV) or T cell receptor beta variable constant region (TCRβC); and
(ii) a second binding domain that specifically binds to a tumor-associated antigen, wherein the tumor-associated antigen is prostate-specific membrane antigen (PSMA).

Embodiment 112. The multi-specific polypeptide molecule of embodiment [001023] wherein the first binding domain comprises the IMGT HCDR1, HCDR2, HCDR3; and the IMGT LCDR1, LCDR2, and LCDR3 comprising the amino acid sequences selected from any one of SEQ ID NOs: 114-116, 1186, 1197, 1201, 2245-2247, 2257-2259, 113, SSS, 73, 1182, YTS, 1157, 2248, AAS, 2249, 2260, and 2261.

Embodiment 113. The multi-specific polypeptide molecule of embodiment [001023] wherein the first binding domain comprises the Kabat HCDR1, HCDR2, HCDR3, and the Kabat LCDR1, LCDR2, and LCDR3 comprising the amino acid sequences selected from any one of SEQ ID NOs: 112, 69-70, 1160, 1171, 1180, 2250-2252, 2262-2264, 71-73, 1148, 1156, 1157, 2253, 2254, 2249, 2265, 2266, and 2261.

Embodiment 114. The multi-specific polypeptide molecule of embodiment [001024], wherein the VH region of the first binding domain comprises the amino acid sequence that is at least about 80%, at least about 85%, at least about 90%, at least about 95%, at least about 97%, at least about 98%, at least about 99%, or 100% identical to any one of SEQ ID NOs: 66, 1113, 2255, and 2267, and wherein the VL region of the first binding domain comprises the amino acid sequence that is at least about 80%, at least about 85%, at least about 90%, at least about 95%, at least about 97%, at least about 98%, at least about 99%, or 100% identical to any one of SEQ ID NOs: 67, 1100, 2256, and 2268.

Embodiment 115. The multi-specific polypeptide molecule of any one of embodiments [001023] to [001026], wherein the first binding domain is a scFv, wherein the scFv comprises the amino acid sequence that is at least about 80%, at least about 85%, at least about 90%, at least about 95%, at least about 97%, at least about 98%, at least about 99%, or 100% identical to any one of SEQ ID NOs: 2295, 149, 1203, 2296, 2297, and 2298.

Embodiment 116. The multi-specific polypeptide molecule of any one of embodiments [001023] to [001027], wherein the second binding domain comprises the IMGT HCDR1, HCDR2, HCDR3, and the IMGT LCDR1, LCDR2, and LCDR3 comprising the amino acid sequence selected from any one of SEQ ID NOs: 2299-2301, 2260, EAS, and 2302.

Embodiment 117. The multi-specific polypeptide molecule of any one of embodiments [001023] to [001027], wherein the second binding domain comprises the Kabat HCDR1, HCDR2, HCDR3, and the Kabat LCDR1, LCDR2, and LCDR3 comprising the amino acid sequence selected from any one of SEQ ID NOs: 2303-2305, 2306, 2307, and 2302.

Embodiment 118. The multi-specific polypeptide molecule of any one of embodiments 111 to 117, wherein the second binding domain comprises a Fab, wherein the Fab heavy chain comprises the amino acid sequence that is at least about 80%, at least about 85%, at least about 90%, at least about 95%, at least about 97%, at least about 98%, at least about 99%, or 100% identical to any one of SEQ ID NOs: 57, and wherein the Fab light chain comprises the amino acid sequence that is at least about 80%, at least about 85%, at least about 90%, at least about 95%, at least about 97%, at least about 98%, at least about 99%, or 100% identical to any one of SEQ ID NOs: 54.

Embodiment 119. The multi-specific polypeptide molecule of any one of embodiments [001023] to [001030], wherein the multi-specific polypeptide molecule comprises two polypeptides, wherein the first polypeptide comprises the amino acid sequence that is at least about 80%, at least about 85%, at least about 90%, at least about 95%, at least about 97%, at least about 98%, at least about 99%, or 100% identical to any one of the first polypeptides of PC01 to PC38; and wherein the second polypeptide comprises the amino acid sequence that is at least about 80%, at least about 85%, at least about 90%, at least about 95%, at least about 97%, at least about 98%, at least about 99%, or 100% identical to any one of second polypeptides of PC01 to PC38.

Embodiment 120. A recombinant nucleic acid molecule encoding the polypeptide molecule of any one of embodiments [00913] to [00965] or the multi-specific polypeptide molecule of any one of embodiments [00967] to [001031].

Embodiment 121. A vector comprising the recombinant nucleic acid molecule of embodiment [001032].

Embodiment 122. A pharmaceutical composition comprising (i) the polypeptide molecule of any one of embodiments [00913] to [00965], the multi-specific polypeptide molecule of any one of embodiments [00967] to [001031], the recombinant nucleic acid molecule of embodiment [001032], or the vector of embodiment [001033], and (ii) a pharmaceutically acceptable carrier, diluent, or excipient.

Embodiment 123. A method of producing a polypeptide, comprising introducing the recombinant nucleic acid molecule of embodiment [001032], or the vector of embodiment [001033] into a host cell under conditions to express the polypeptide molecule or the multi-specific polypeptide molecule in the cell, wherein the method is ex vivo.

Embodiment 124. A method of expanding a T cell population, wherein the method comprises contacting the T cell population with the polypeptide molecule of any one of embodiments [00913] to [00965], the multi-specific polypeptide molecule of any one of embodiments [00967] to [001031], or the pharmaceutical composition of embodiment [001034], wherein the T cell population express the TCRβV on the cell surface, and wherein the polypeptide molecule or the first binding domain of the multi-specific polypeptide molecule specifically binds to the TCRβV.

Embodiment 125. The method of embodiment [001036], wherein the method expands T cells in vitro or ex vivo.

Embodiment 126. The method of embodiment [001036], wherein the method expands T cells in vivo in a subject.

Embodiment 127. The method of any one of embodiments [001036] to [001038] wherein the population of T cells comprises a plurality of effector T cells and/or memory T cells.

Embodiment 128. The method of any one of embodiments [001036] to [001039] wherein the population of T cells has an increased expansion of the effector T cells and/or memory T cells than that produced by a control polypeptide comprising a binding domain that specifically binds to CD3 instead of the TCRβV.

Embodiment 129. The method of embodiment [001040], wherein the effector T cells are selected from the group consisting of effector memory T cells (T_{EM}), T effector cells (T_{EFF}), and T effector cells re-expressing CD45RA (T_{EMRA}), or are a combination thereof.

Embodiment 130. The method of embodiment [001040], wherein the memory T cells are selected from the group consisting of central memory T cells (T_{CM}) and T memory stem cells (T_{SCM}), or are a combination thereof.

Embodiment 131. The method of any one of embodiments [001036] to [001042] wherein the population of T cells has an increased percentage of effector T cells and/or memory T cells than that produced by a control polypeptide comprising a binding domain that specifically binds to CD3 instead of the TCRβV.

Embodiment 132. The method of embodiment [001043], wherein the effector T cells are effector memory T cells (T_{EM}).

Embodiment 133. The method of embodiment [001043], wherein the memory T cells are central memory T cells (T_{CM}).

Embodiment 134. The method of any one of embodiments [001036] to [001045] wherein the expanded T cell population has an increased and/or prolonged cytotoxicity than that of a control polypeptide comprising a binding domain specifically binds to CD3 instead of the TCRβV.

Embodiment 135. The method of any one of embodiments [001040] to [001046] wherein the comparison between the population of T cells expanded by the polypeptide molecule or the multi-specific polypeptide molecule and the control polypeptide is performed at day 1, 2, 3, 4, 5, 6, 7, 8, 9, 10, or 11 after an initial contact of the T cell population.

Embodiment 136. A method of depleting target cells in a subject, comprising administering to the subject an effective amount of the polypeptide molecule of any one of embodiments [00913] to [00965], the multi-specific polypeptide molecule of any one of embodiments [00967] to [001031], or the pharmaceutical composition of embodiment [001034] wherein the target cells are tumor cells.

Embodiment 137. A method of depleting target cells in a subject, comprising administering to the subject an effective amount of the polypeptide molecule of any one of embodiments [00913] to [00965], the multi-specific polypeptide molecule of any one of embodiments [00967] to [001031], or the pharmaceutical composition of embodiment [001034] wherein the target cells are B cells.

Embodiment 138. The method of embodiment [001048] or [001049], wherein the method decreases the number of target cells by at least about 50%, at least about 55%, at least about 60%, at least about 65%, at least about 70%, at least about 75%, at least about 80%, at least about 85%, at least about 90%, at least about 95%, at least about 98%, at least about 99%, at least about 99.5%, at least about 99.8%, at least about 99.9%, or 100% in the subject.

Embodiment 139. A method of treating a disease or disorder in a subject in need thereof, comprising administering to the subject an effective amount of the polypeptide molecule of any one of embodiments [00913] to [00965], the multi-specific polypeptide molecule of any one of embodiments [00967] to [001031], or the pharmaceutical composition of embodiment [001034].

Embodiment 140. The method of embodiment [001051], wherein the disease or disorder is a cancer.

Embodiment 141. The method of embodiment [001052], wherein the cancer is a blood cancer.

Embodiment 142. The method of embodiment [001053], wherein the blood cancer is chronic lymphocytic leukemia or non-Hodgkin lymphoma.

Embodiment 143. The method of embodiment [001052], wherein the cancer is a solid tumor.

Embodiment 144. The method of embodiment [001051], wherein the disease or disorder is an autoimmune disease.

Embodiment 145. The method of embodiment [001056], wherein the autoimmune disease is rheumatoid arthritis, Systemic lupus erythematosus, or Myasthenia Gravis.

Embodiment 146. The method of any one of embodiments [001051] to [001057] wherein the subject has previously been treated with a polypeptide comprising a binding domain that specifically binds to CD3 instead of the TCRβV.

Embodiment 147. The method of any one of embodiments [001051] to [001058] wherein the subject is relapsing or refractory to a treatment comprising a polypeptide comprising a domain that specifically binds to CD3 instead of the TCRβV.

### EXAMPLES

TABLE 35
Example Navigation

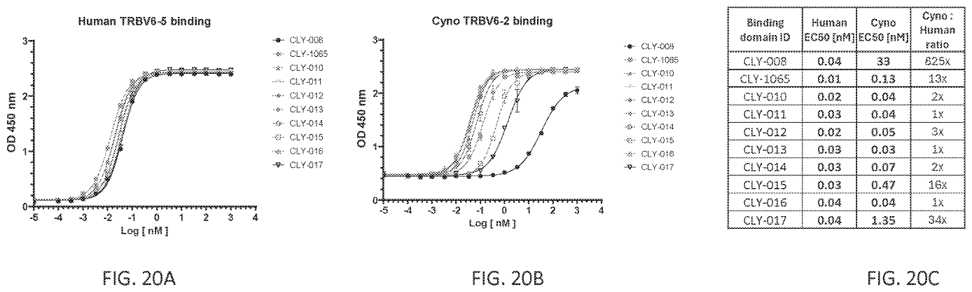

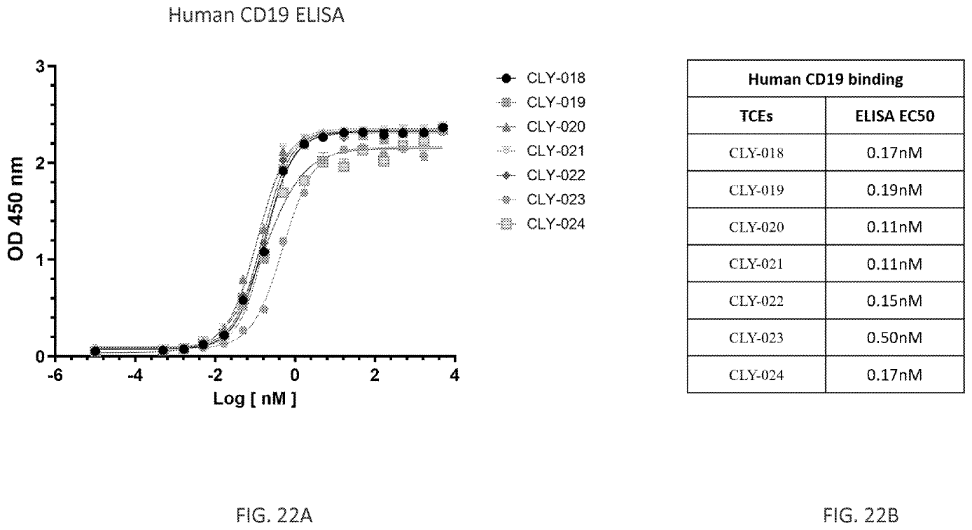

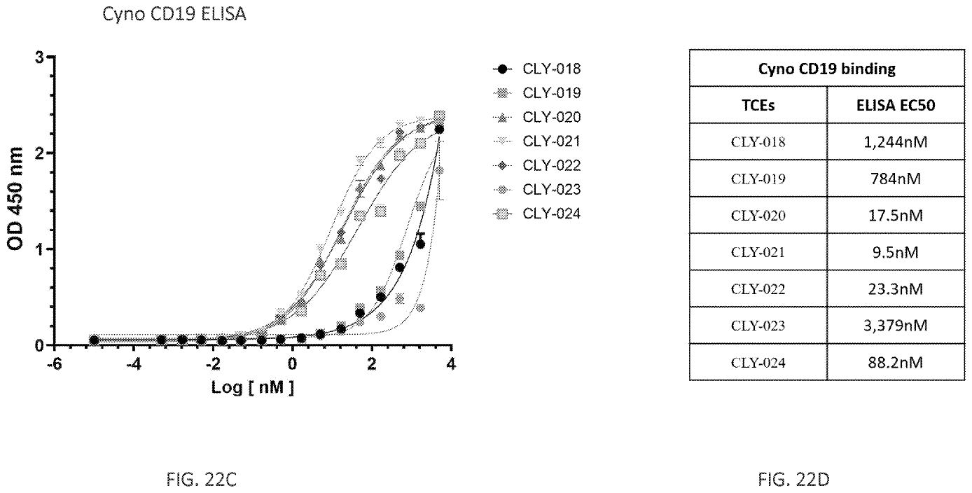

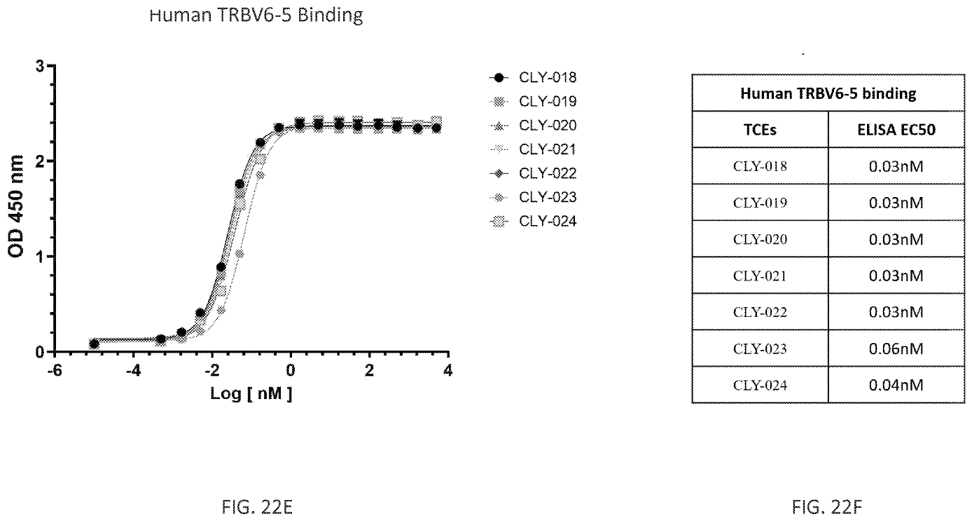

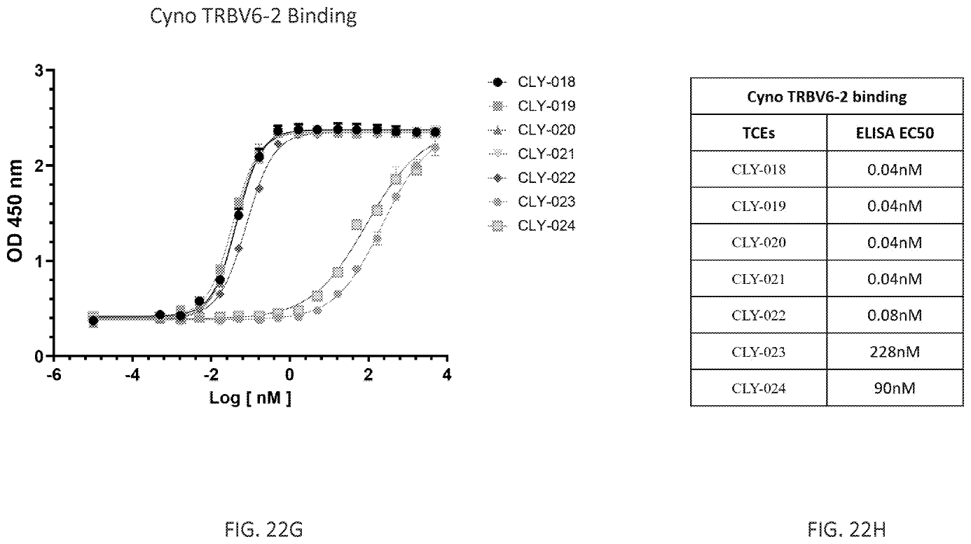

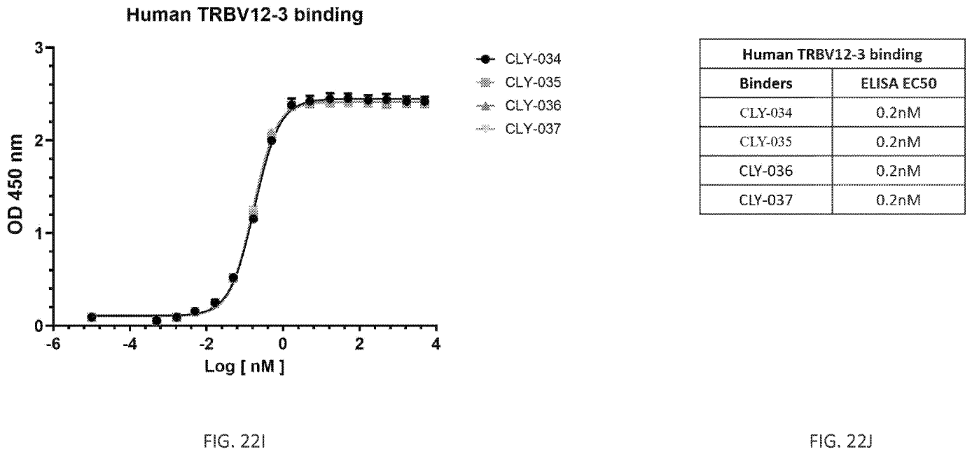

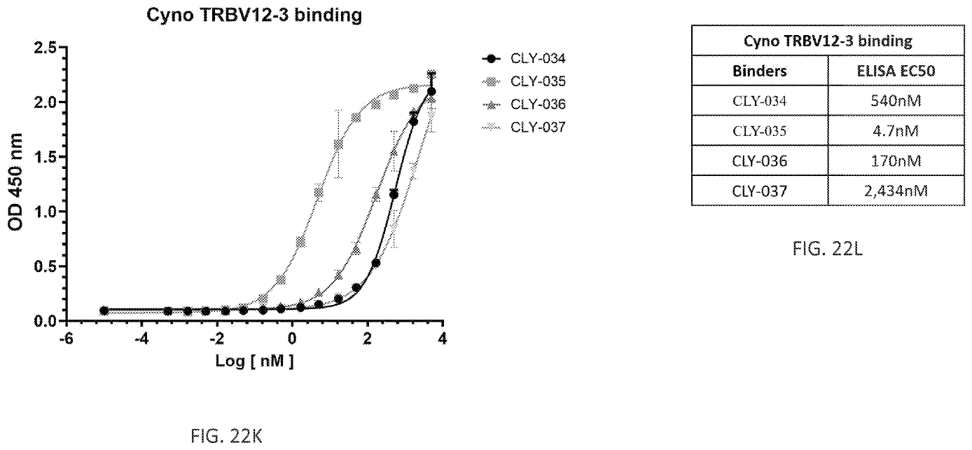

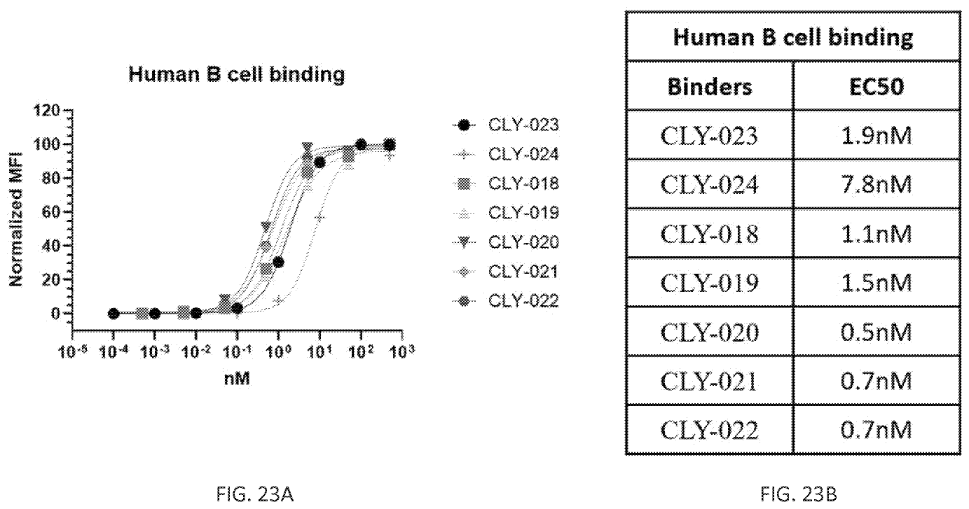

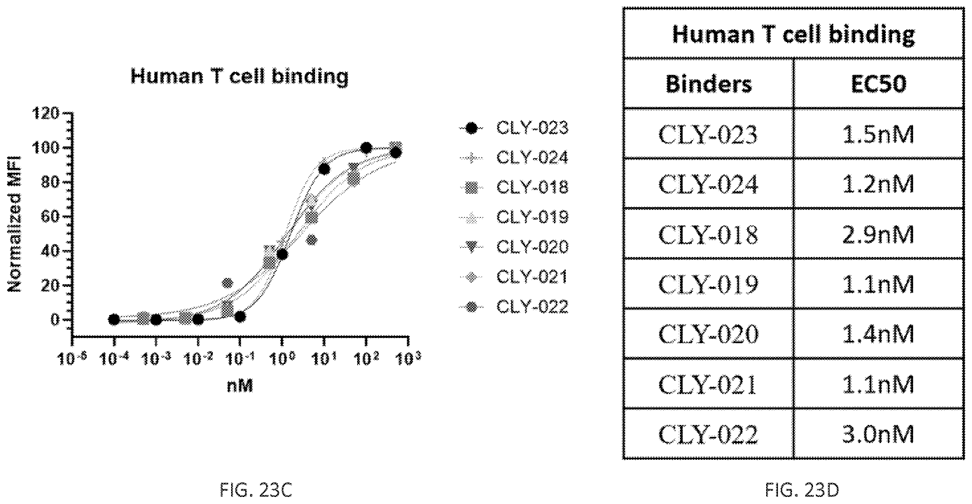

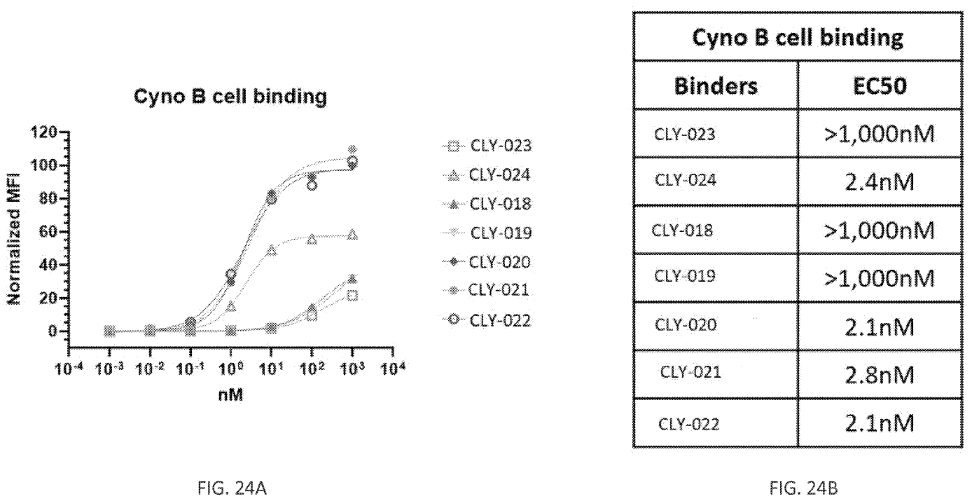

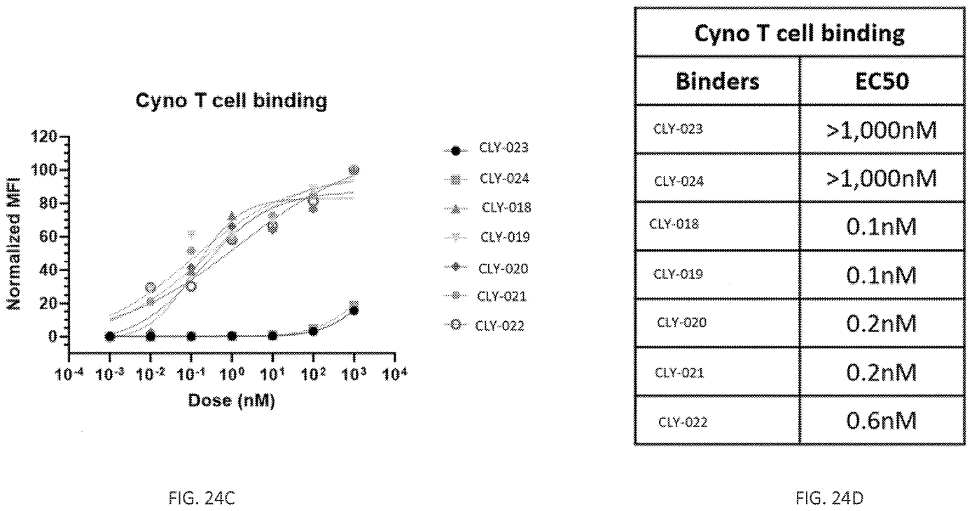

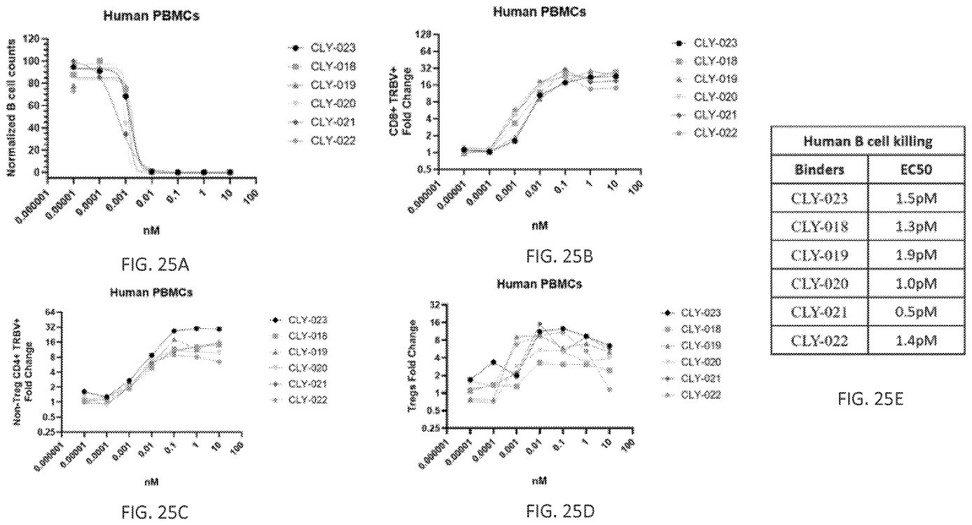

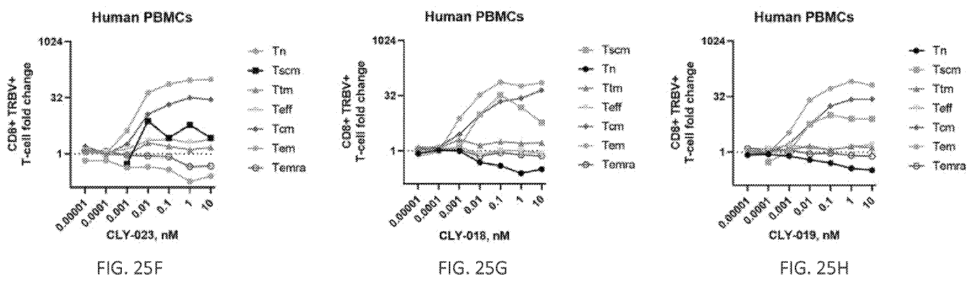

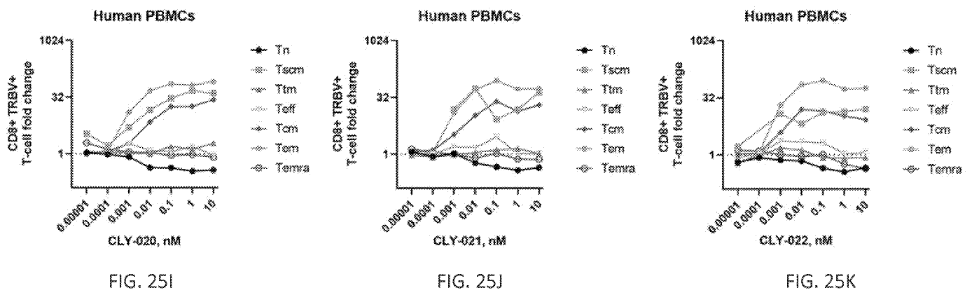

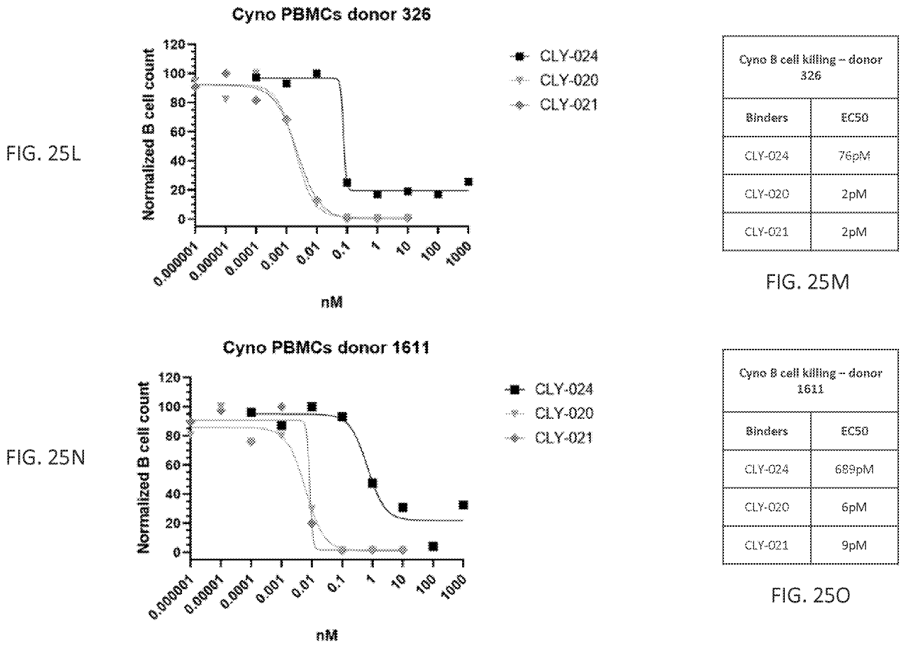

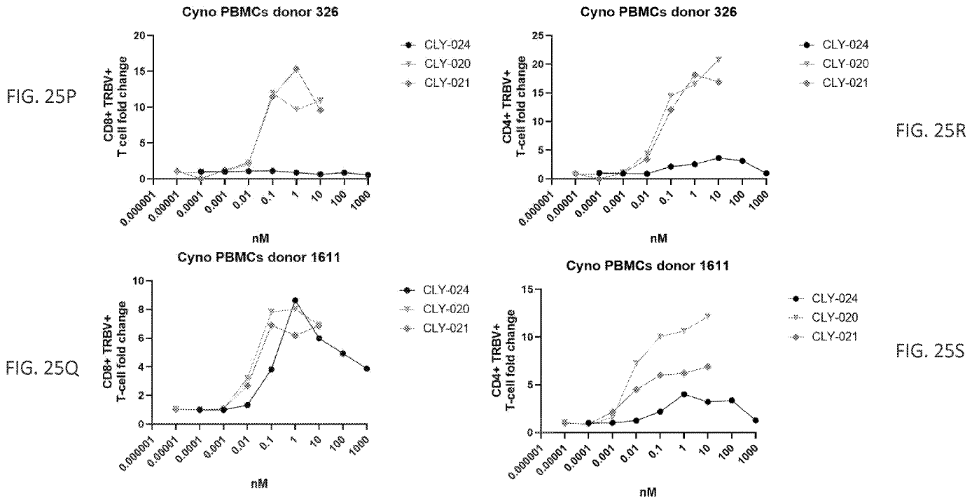

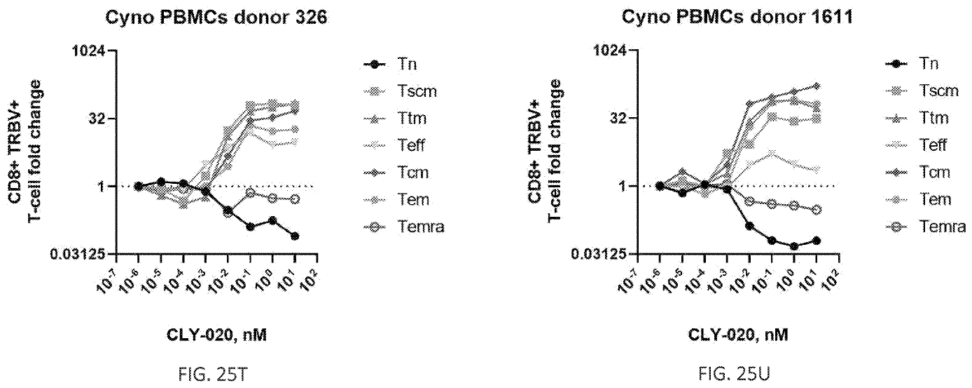

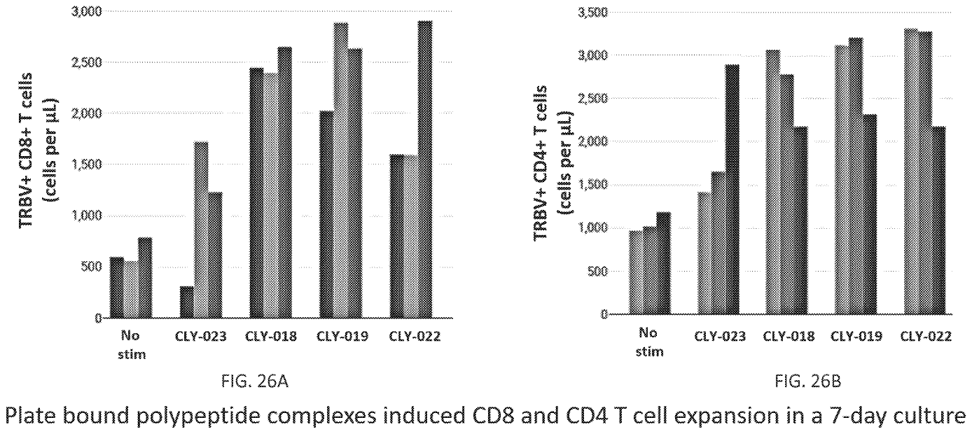

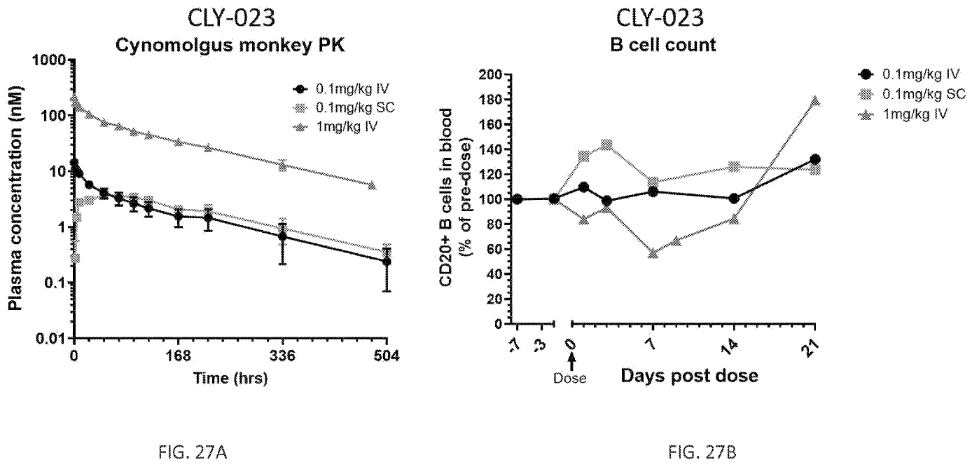

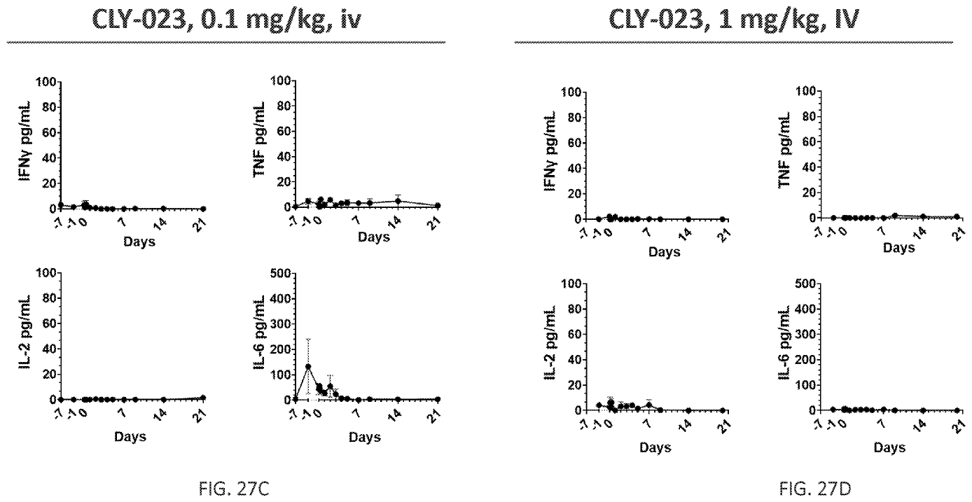

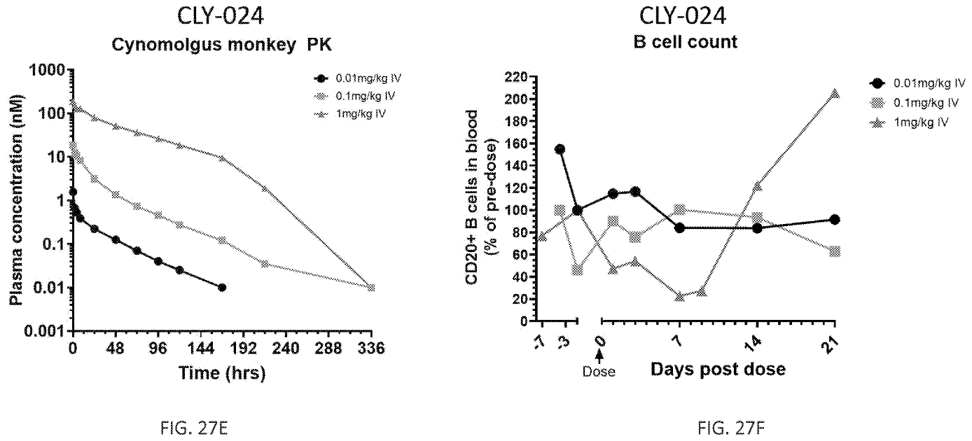

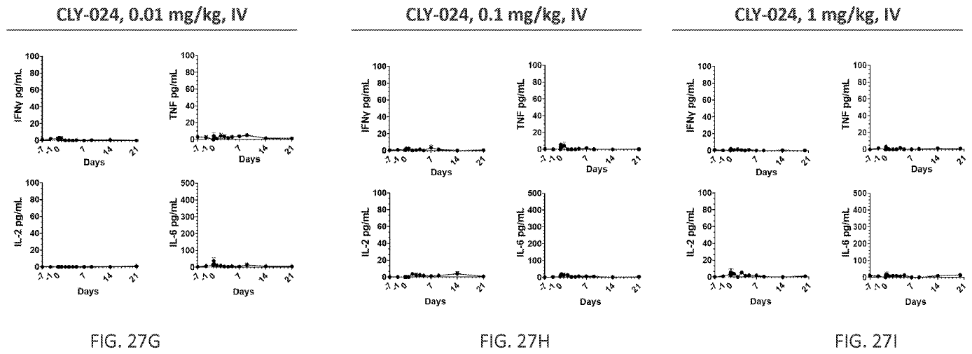

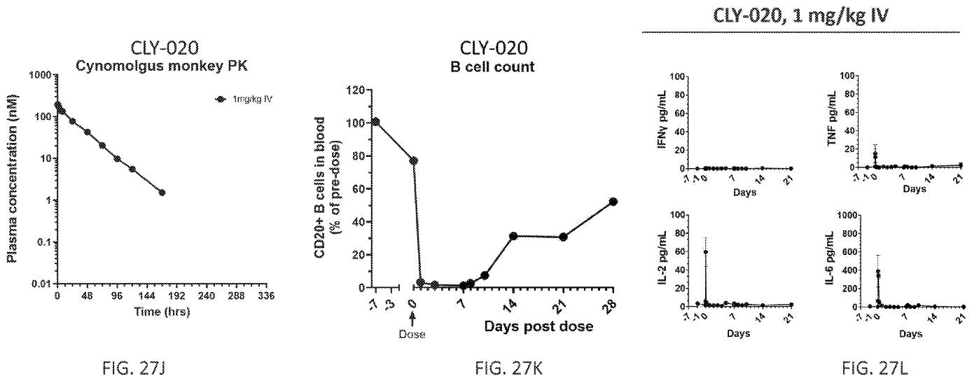

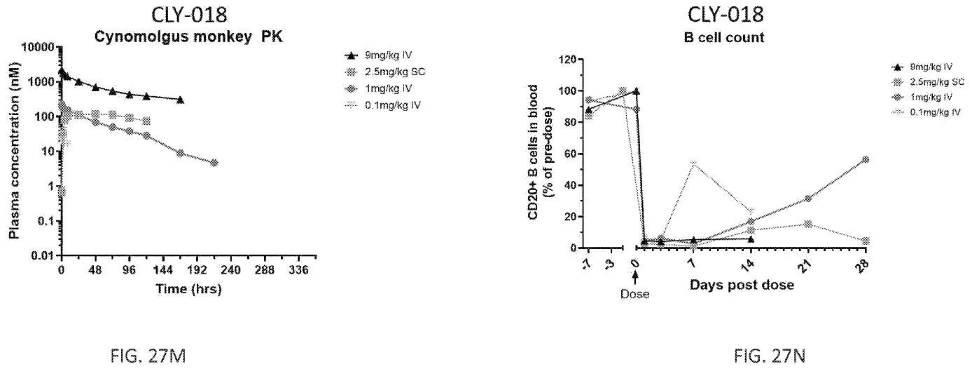

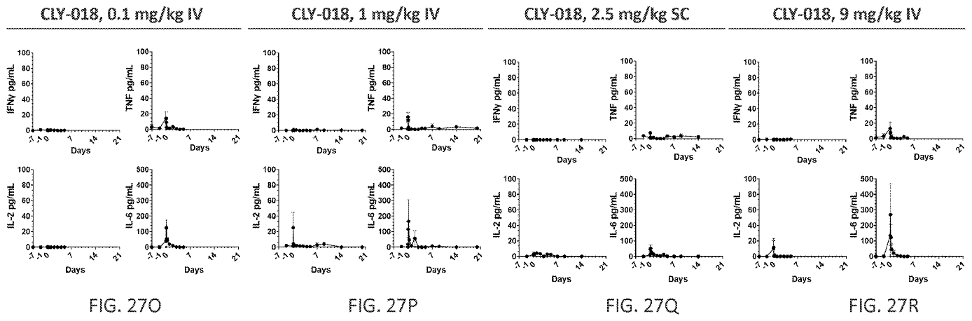

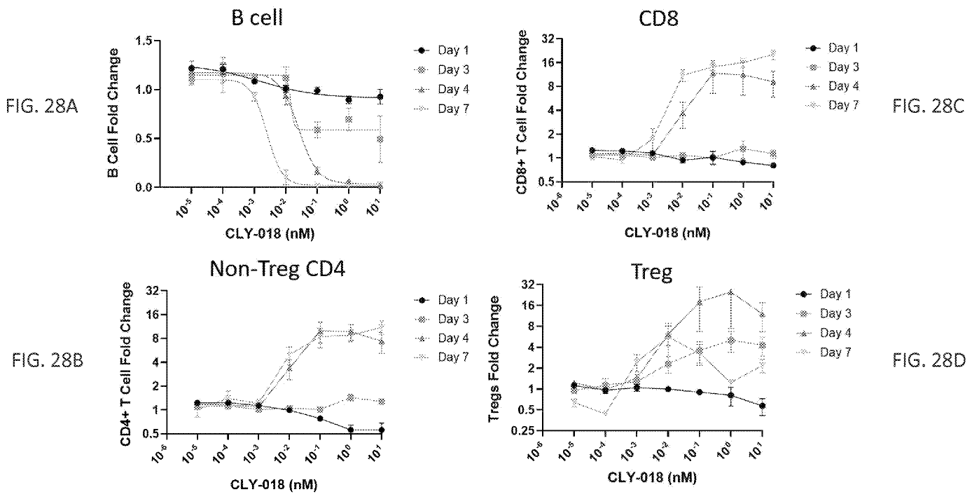

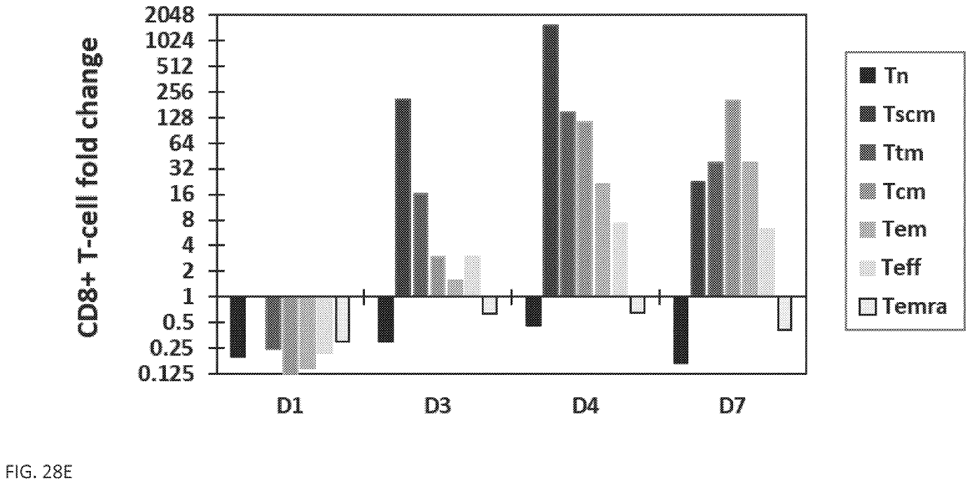

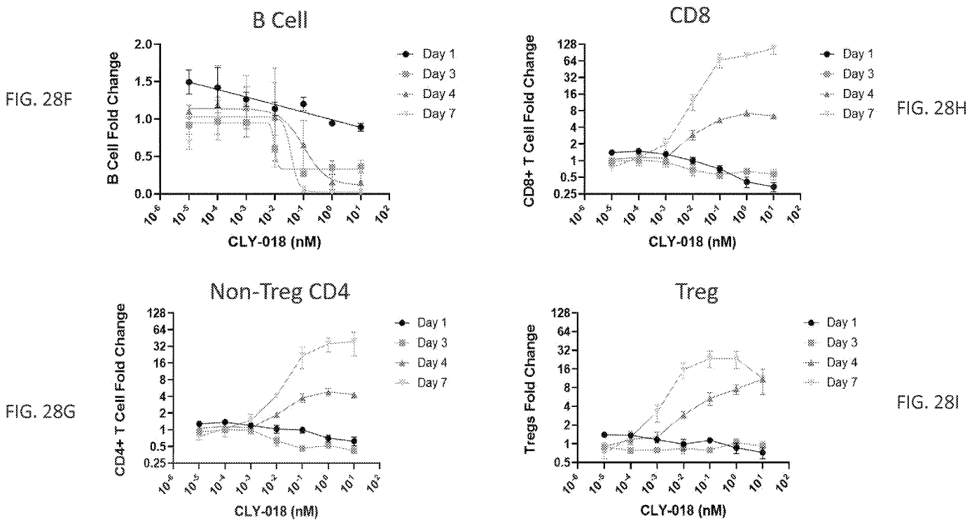

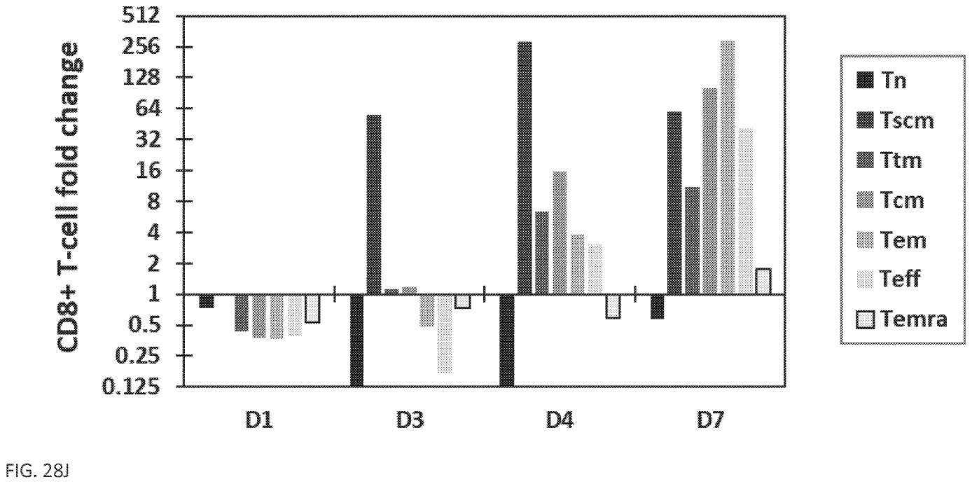

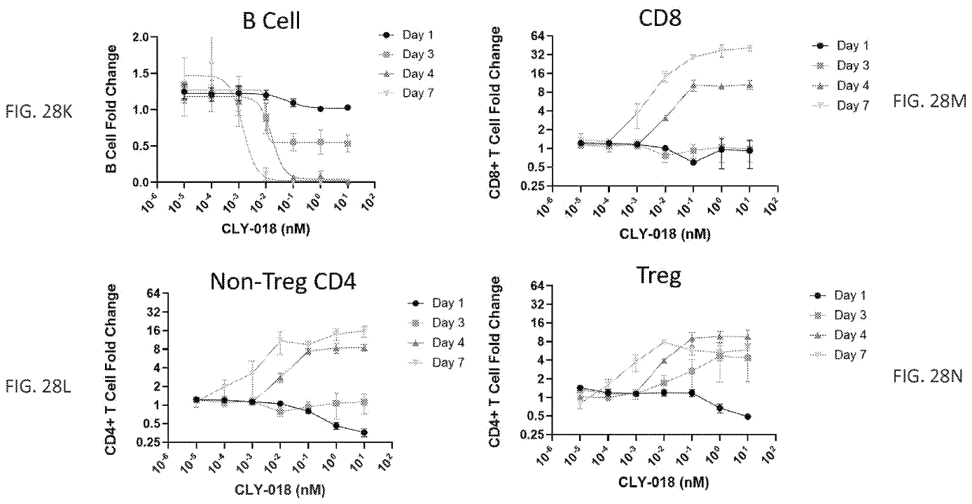

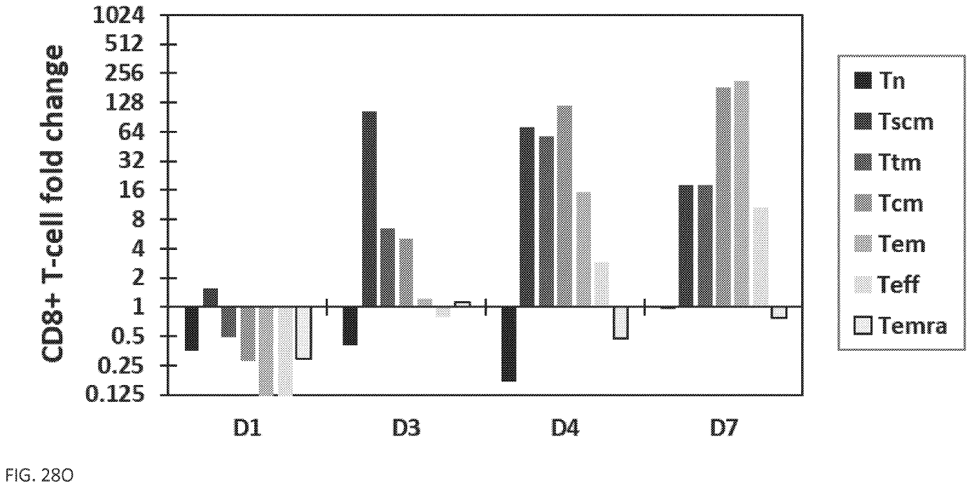

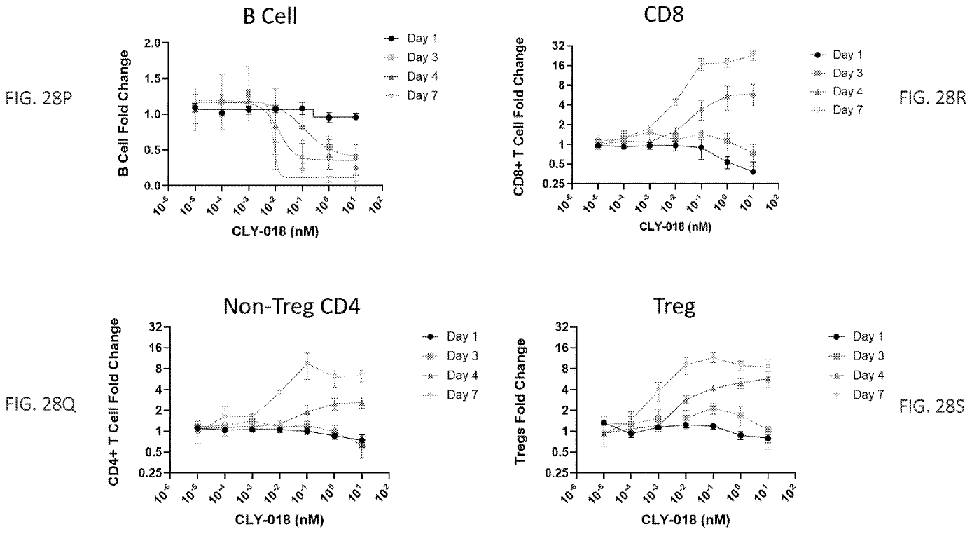

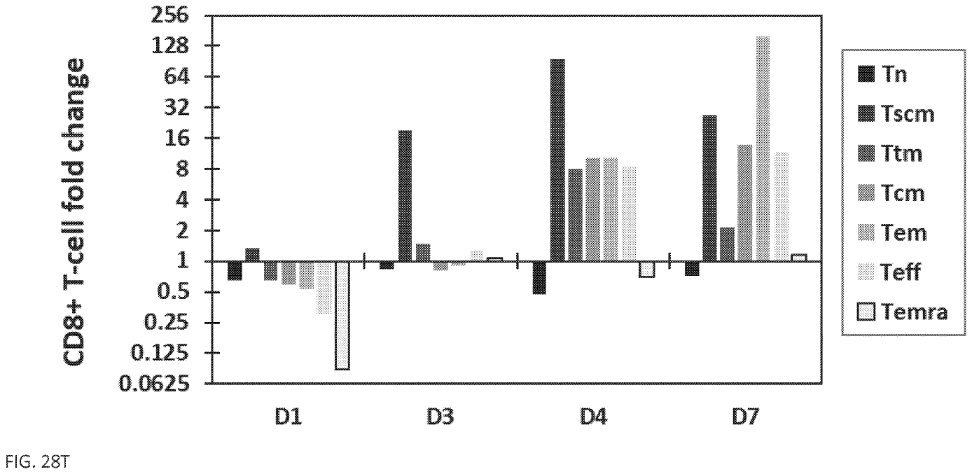

Example 1
Polypeptide Molecules Bispecific to PSMA and
TCRβV or TCRβC Demonstrate Effective
Tumor Cell Killing and Reduced Cytokine Release
In Vitro. Molecules targeting TCR result in
expansion of T_{cm} subset, which is not a feature
of CD3-binding counterparts.
Example 2
Design and generation of mutational libraries of
TCRβV6-5 binding domains and screening via
ELISA and Bioacore. Combinatorial testing
and generation of novel ScFv and Fab fragments.
Expression of library in CHO cells. Modified T
cell binding domains exhibited 2-fold stronger
human TCRβV6-5 binding affinity as shown
in FIG. 20A, as well as drastically strengthened
cyno TCRβV6-2 binding by more than
800-fold as shown in FIG. 20B.
Example 3
Design and generation of TRβV12-3 mutational
libraries and screening. Generation of novel
scFv and Fab fragments. Expression of library
in CHO. Modified T cell binding domains CLY-
035 and CLY-036 exhibit improved binding
against cyno TRBV12-3 at FIGS. 22K-22L without
negative impacts to human TRBV12-3 binding,
as shown at FIGS. 221-22J.
Example 4
Design and generation of bispecific polypeptide
molecules. TCRβV6-5 binding domains CLY-
018 to CLY-024 were incorporated into
bispecifics with second CD19 binding domain (i.e.,
producing CD19XTCRβV polypeptide molecules).
These bispecifics were tested for binding to
human and cyno CD19, and for binding
against human TCRβV6-5 or cyno TCRβV6-2.
Bispecific polypeptide molecules with mutated
T cell binders exhibit improved binding
properties against human (FIGS. 23C-23D)
and cyno (FIGS. 24C-24D) TCRβV.
Example 5:
CD19xTRBV polypeptide molecules containing
CLY-018-CLY-023 were evaluated for human
B cell killing (FIG. 25A) and expansion
(FIG. 25B-K). Corresponding experiments for
cyno B cell killing (FIGS. 25L-O), and expansion
(FIGS. 25P-S) were also conducted. The memory
full phenotype of cyno cells transformed with
the CD19xTRBV bispecific polypeptide molecule
CLY-020 is displayed at FIGS. 25T-U. T cell
population expansion is displayed across FIGS.
26A-F.
Example 6
Cynomolgus monkeys dosed with polypeptide
molecules with Modified T cell binding domains
from earlier examples exhibited reduced
B-cell counts, and no signs of cytokine release
syndrome. Results for CLY-024 are displayed
at FIGS. 27E-27F. Results for CLY-023 are
displayed at FIGS. 27A- 27B. Results for
polypeptide molecules comprising CLY-020 are
displayed at 27J-27L. Results for polypeptide
molecules comprising CLY-028 are displayed at
FIGS. 27O-27R.
Example 7
CD19xTRBV bispecific polypeptide molecules
containing CLY-018 was evaluated for human
B cell killing and T cell expansion in healthy and
autoimmune disease PBMCs. Results for
healthy donors presented in FIGS. 28A-28E.
Results for Rheumatoid Arthritis subjects
displayed at FIGS 28F-28J. Results for Systemic
Lupus subjects displayed at FIGS. 28K-28O.
Results for Myasthenia Gravis subjects
displayed at FIGS. 28P-28T.

### Example 1. Anti-TCRβ/Anti-PSMA Polypeptide Molecules Demonstrate Effective Tumor Cell Killing and Reduced Cytokine Release In Vitro

The objective of this study was to evaluate polypeptide molecules bispecific to PSMA and TCRβV or PSMA and TCRβC in a functional in vitro tumor killing assay using the PSMA-positive prostate cancer cell line LNCaP.

Polypeptide molecules specific to PSMA and TCRβV, TCRβC, or CD3 are listed in Table 8 below.

TABLE 8
Exemplary Anti-TCRβ/Anti-PSMA Polypeptide Molecules

| PC | TCRβ or CD3 | TAA | SEQ ID NO | |
|---|---|---|---|---|
| ID | Target | Target | Chain 1 | Chain 2 |
| PC43 | CD3 | PSMA | 54 | 55 |
| PC44 | CD3 | PSMA | 56 | 57 |
| PC01 | TCRβC1 | PSMA | 54 | 2342 |
| PC02 | TCRβC1 | PSMA | 2343 | 57 |
| PC03 | TCRβC2 | PSMA | 54 | 2344 |
| PC04 | TCRβC2 | PSMA | 2345 | 57 |
| PC05 | TCRβV6-5 | PSMA | 54 | 2346 |
| PC06 | TCRβV6-5 | PSMA | 2347 | 57 |
| PC07 | TCRβV12-3/4 | PSMA | 54 | 2348 |
| PC08 | TCRβV12-3/4 | PSMA | 2349 | 57 |
| PC09 | TCRβV5-5/6 | PSMA | 54 | 2350 |
| PC10 | TCRβV5-5/6 | PSMA | 2351 | 57 |
| PC11 | TCRβV20-1 | PSMA | 54 | 2352 |
| PC12 | TCRβV20-1 | PSMA | 2353 | 57 |

In Vitro Tumor Cell Viability and Cytokine Release Following Treatment with Polypeptide Molecules

Tumor cell viability was measured using an xCelligence real time cell analyzer (Agilent) that relies on sensor impedance measurements (cell index) that increases as tumor cells adhere, spread, and expand on the surface of the sensor and decreases as the tumor cells are killed. Ten thousand tumor cells were added per well and allowed to adhere overnight on a 96 well E-Plate. The following day, compositions comprising polypeptide molecules titrated in human serum supplemented medium and 50,000 CD8+ T cells were added to the wells at CD8+ T cells:LNCaP ratio of 5:1. Cell index measurements were then taken every 10 minutes for 120 hours. The concentration required to reduce the tumor growth 50% (IC50) was calculated by multiplying the cell index by the number of hours (tumor cell growth kinetics) and plotting the resultant value versus concentration of polypeptide molecule (pM) using Graphpad Prism software. Supernatants at the end of the co-culture experiments were collected and analyzed for cytokines (IFNγ, TNF, and IL-2) using a Meso Scale Device kit following the manufacturer's instructions.

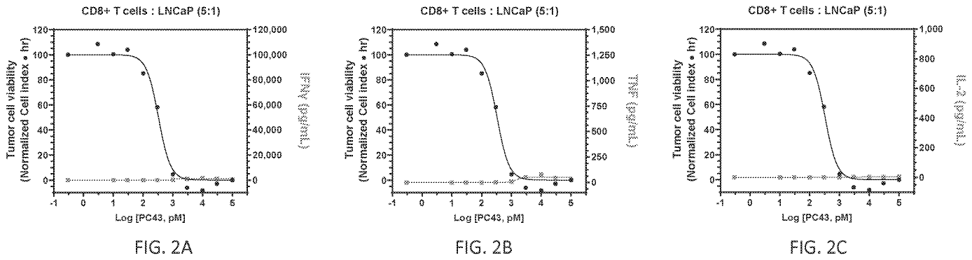

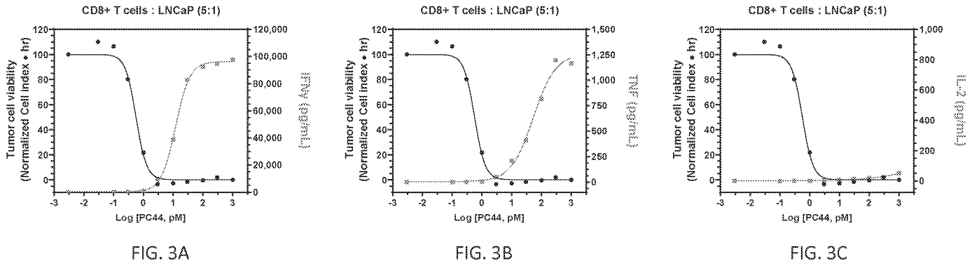

FIGS. 2A-2C and FIGS. 3A-3C show tumor cell viability and IFNγ, TNF, and IL-2 cytokine release with increasing concentrations of the PSMA-CD3 polypeptide molecules PC43 (FIGS. 2A-2C) and PC44 (FIGS. 3A-3C) in a co-culture of CD8+ T cells and LNCaP tumor cells.

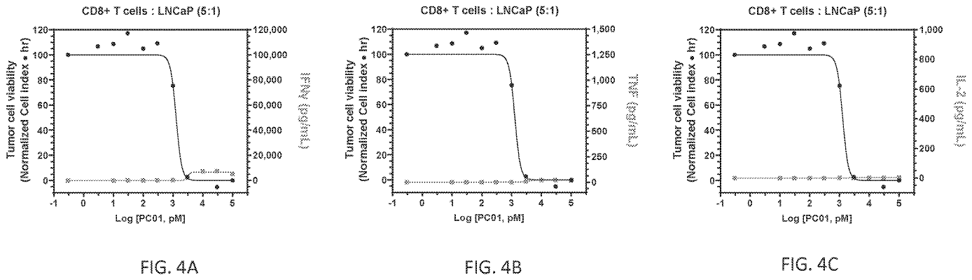

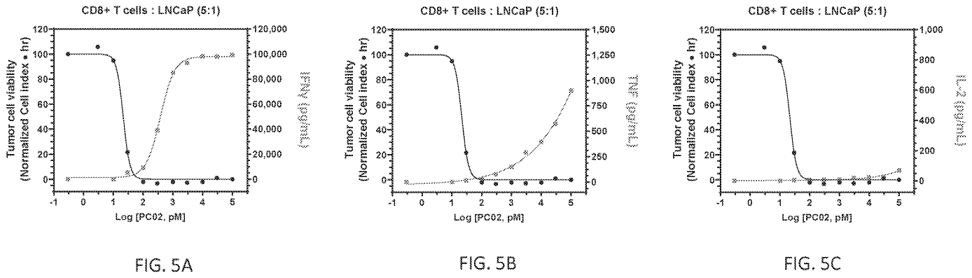

FIGS. 4A-4C and FIGS. 5A-5C show tumor cell viability and IFNγ, TNF, and IL-2 cytokine release with increasing concentrations of the PSMA-TCRβC1 polypeptide molecules PC01 (FIGS. 4A-4C) and PC02 (FIGS. 5A-5C) in a co-culture of CD8+ T cells and LNCaP tumor cells.

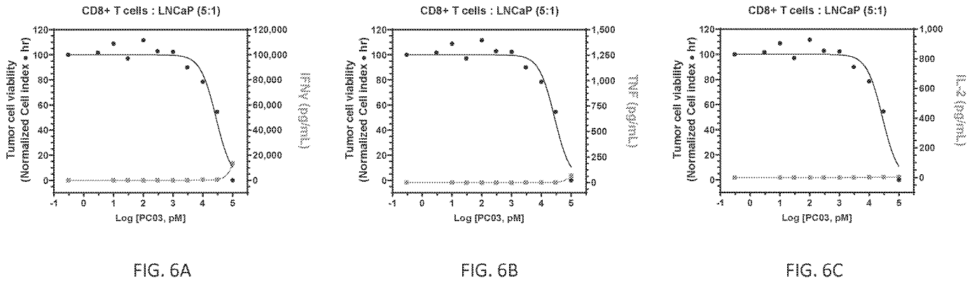

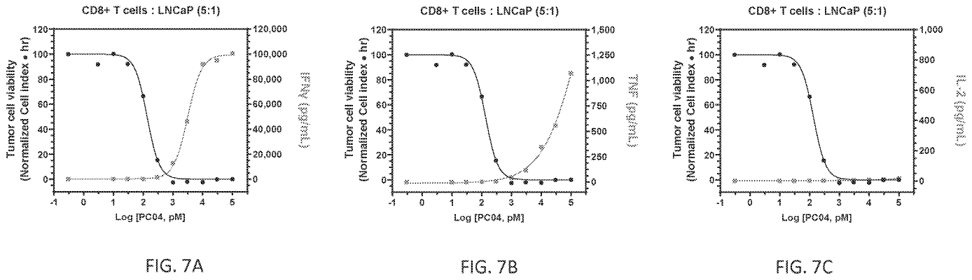

FIGS. 6A-6C and FIGS. 7A-7C show tumor cell viability and IFNγ, TNF, and IL-2 cytokine release with increasing concentrations of the PSMA-TCRβC2 polypeptide molecules PC03 (FIGS. 6A-6C) and PC04 (FIGS. 7A-7C) in a co-culture of CD8+ T cells and LNCaP tumor cells.

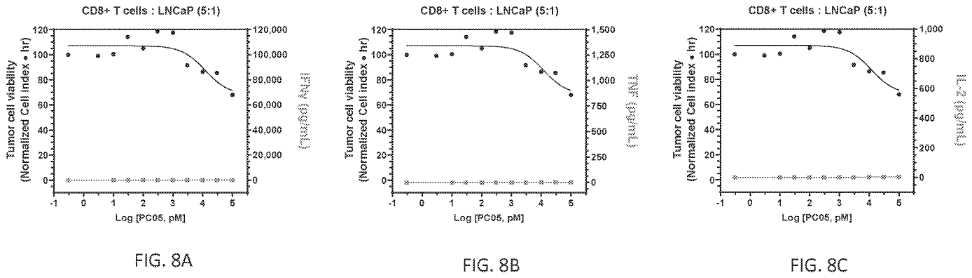

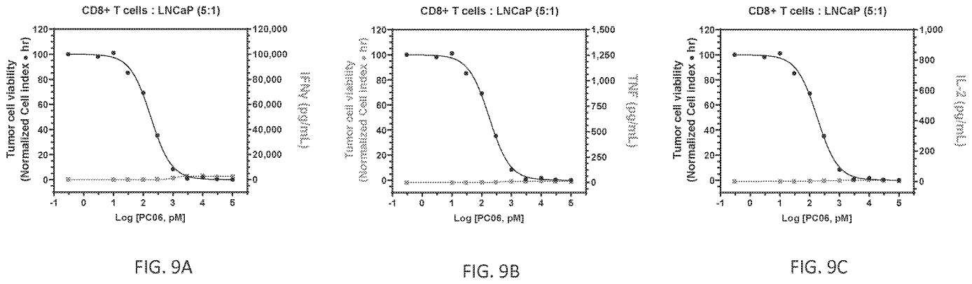

FIGS. 8A-8C and FIGS. 9A-9C show tumor cell viability and IFNγ, TNF, and IL-2 cytokine release with increasing concentrations of the PSMA-TCRβV6-5 polypeptide molecules PC05 (FIGS. 8A-8C) and PC06 (FIGS. 9A-9C) in a co-culture of CD8+ T cells and LNCaP tumor cells.

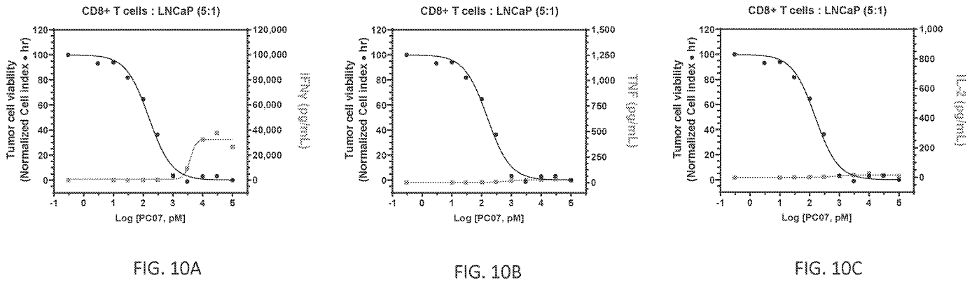

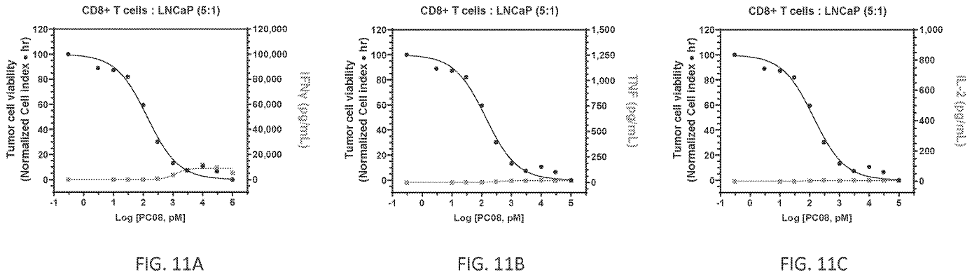

FIGS. 10A-10C and FIGS. 11A-11C show tumor cell viability and IFNγ, TNF, and IL-2 cytokine release with increasing concentrations of the PSMA-TCRβV12-3/4 polypeptide molecules PC07 (FIGS. 10A-10C) and PC08 (FIGS. 11A-11C) in a co-culture of CD8+ T cells and LNCaP tumor cells.

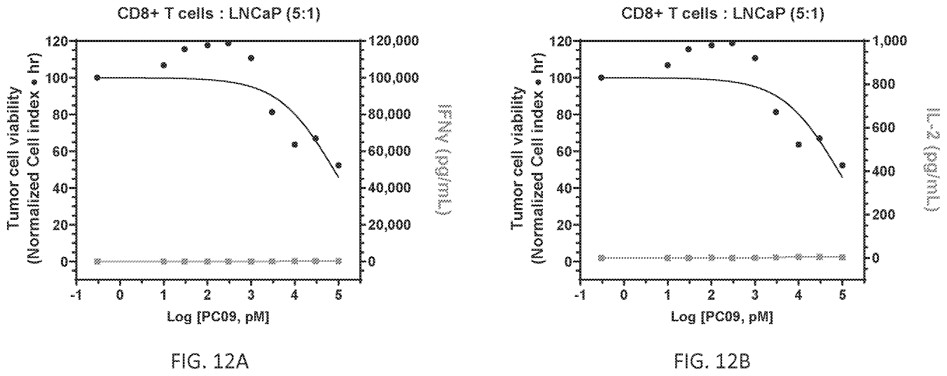

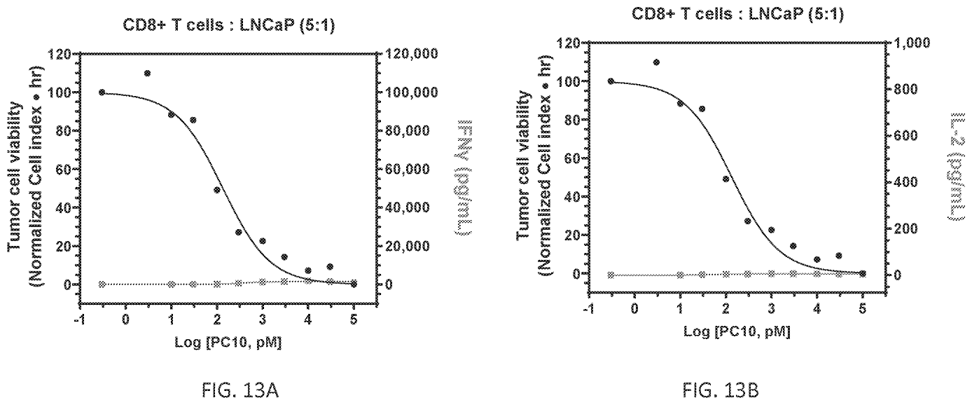

FIGS. 12A-12B and FIGS. 13A-13B show tumor cell viability and IFNγ and IL-2 cytokine release with increasing concentrations of the PSMA-TCRβV5-5/6 polypeptide molecules PC09 (FIGS. 12A-12B) and PC10 (FIGS. 13A-13B) in a co-culture of CD8+ T cells and LNCaP tumor cells.

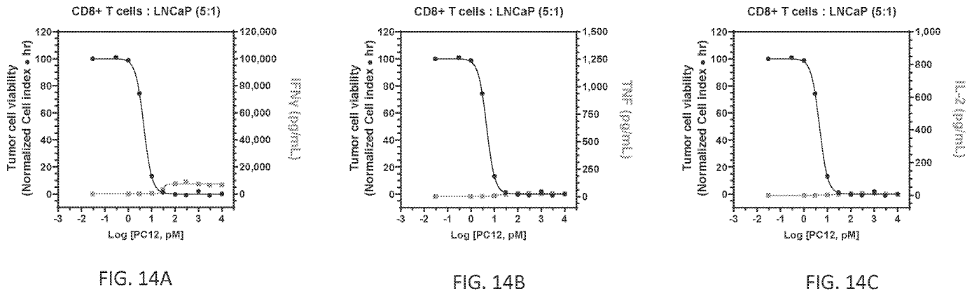

FIGS. 14A-14C show tumor cell viability and IFNγ, TNF, and IL-2 cytokine release with increasing concentrations of the PSMA-TCRβ20-1 polypeptide molecules PC12 in a co-culture of CD8+ T cells and LNCaP tumor cells.

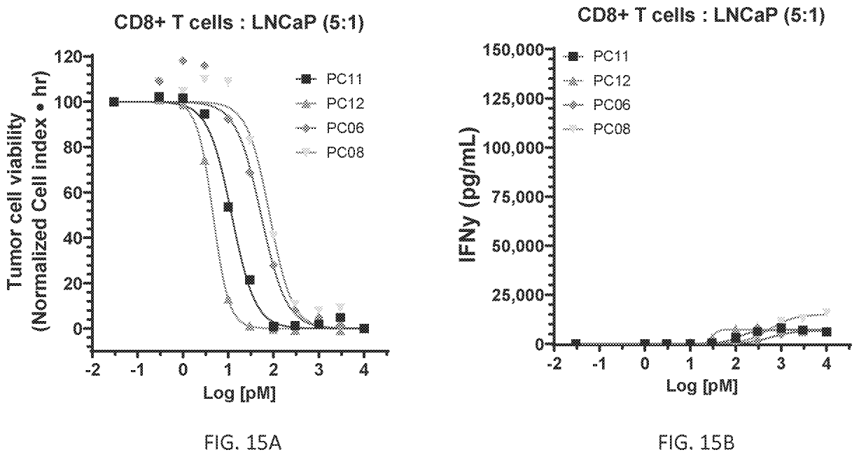

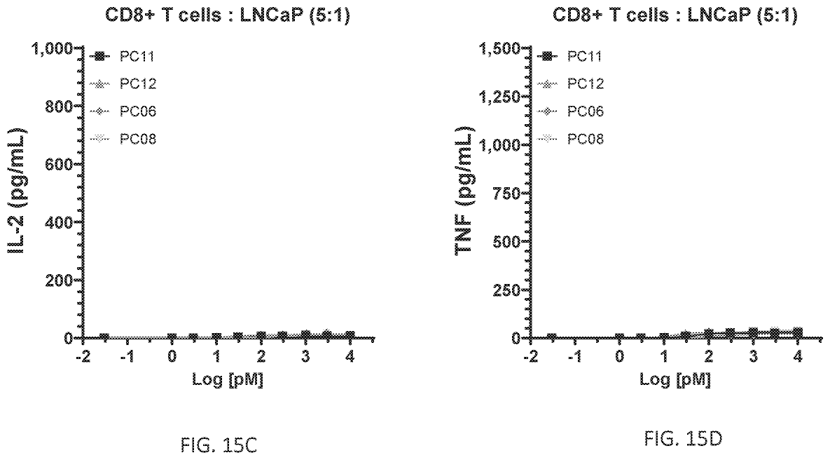

FIGS. 15A-15D show tumor cell viability and IFNγ, TNF, and IL-2 cytokine release with increasing concentrations of PSMA-TCRβ20-1 (PC11 and PC12), PSMA-TCRβ12-3/4 (PC08), and PSMA-TCRβ6-5 (PC06) polypeptide molecules in a co-culture of CD8+ T cells and LNCaP tumor cells.

The calculated EC50 values for the polypeptide molecules are provided in Table 9 below.

TABLE 9
EC50 Values for Polypeptide Molecules

| Polypeptide | EC50 | TCRβ or | TAA |
|---|---|---|---|
| Molecule | (pM) | CD3 Target | Target |
| PC43 | 328 | CD3 | PSMA |
| PC44 | 0.5 | CD3 | PSMA |
| PC11 | ND | TCRβV20-1 | PSMA |
| PC12 | 5 | TCRβV20-1 | PSMA |
| PC01 | 1,277 | TCRβC1 | PSMA |
| PC02 | 21 | TCRβC1 | PSMA |
| PC03 | 27,663 | TCRβC2 | PSMA |
| PC04 | 140 | TCRβC2 | PSMA |
| PC05 | ND | TCRβV6-5 | PSMA |
| PC06 | 180 | TCRβV6-5 | PSMA |
| PC07 | 155 | TCRβV12-3/4 | PSMA |
| PC08 | 135 | TCRβV12-3/4 | PSMA |
| PC09 | ND | TCRβV5-5/6 | PSMA |
| PC10 | 87 | TCRβV5-5/6 | PSMA |

Overall, these results support the effectiveness of bispecific polypeptide molecules targeting PSMA and TCRβV or TCRβC in tumor cell killing in vitro. Notably, the polypeptide molecules targeting PSMA and TCRβV or TCRβC also demonstrated reduced IFNγ, TNF, and IL-2 cytokine release compared to polypeptide molecules targeting PSMA and CD3. These results suggest that polypeptide molecules targeting PSMA and TCRβV or TCRβC are efficacious and may produce less toxic side effects associated with inflammatory cytokine release.

### Example 2: Design and Generation of T Cell Receptor Beta Variable 6-5 (TCRβV6-5) Polypeptide Molecules

### A. Saturating Mutagenesis

TCRβV6-5-binding domains were generated by systematically mutating all CDRs of a wildtype TCRβV6-5-binding domain disclosed in CHOI et al., Proc. Natl. Acad. Sci., 88, 8357-8361(1991) to identify favorable mutations and combinations of mutations to discover novel and improved TCRβV6-5-binding domains with unique CDRs. The wildtype TCRβV6-5-binding domain is shown in Table 10 below. Saturating mutagenesis was performed on the underlined amino acid residues in Table 10.

TABLE 10
Wildtype TCRβV6-5-binding Domain

| SEQ ID | | |
|---|---|---|
| NO | Description | Sequence |
| 66 | VH | QVQLVQSGAEVKKPGSSVKVSCKASGYSFTTYYIHWVRQA |
| | | PGQGLEWMGWFFPGSGNIKYNEKFKGRVTITADTSTSTAY |
| | | MELSSLRSEDTAVYYCAGSYYSYDVLDYWGQGTTVTVSS |
| 67 | VL | DIQMTQSPSFLSASVGDRVTITCKASQNVGINVVWHQQKP |
| | | GKAPKALIYSSSHRYSGVPSRFSGSGSGTEFTLTISSLQPEDF |
| | | ATYFCQQFKSYPLTFGQGTKLEIK |
| 68 | VH CDR1 | GYSFTTYYIH |
| 112 | Kabat | TYYIH |
| | VH CDR1 | |
| 114 | IMGT VH | GYSFTTYY |
| | CDR1 | |
| 69 | Kabat | WFFPGSGNIKYNEKFKG |
| | VH CDR2 | |
| 115 | IMGT | FFPGSGNI |
| | VH CDR2 | |
| 70 | Kabat VH | SYYSYDVLDY |
| | CDR3 | |
| 116 | IMGT | AGSYYSYDVLDY |
| | VH CDR3 | |
| 71 | Kabat VL | KASQNVGINVV |
| | CDR1 | |
| 113 | IMGT | QNVGIN |
| | VL CDR1 | |
| 72 | Kabat VL | SSSHRYS |
| | CDR2 | |
| Not | IMGT | SSS |
| Applicable | VL CDR2 | |
| 73 | Kabat VL | QQFKSYPLT |
| | CDR3 | |
| 73 | IMGT | QQFKSYPLT |
| | VL CDR3 | |

Mutant full length VH or VL domains with saturating single point mutations in individual CDR positions were then assembled via high fidelity polymerase chain reaction (PCR) techniques and appropriately designed primers and terminal restriction sites. Full length mutant VH or VL domains were then cloned into the appropriate vectors designed to express and secrete Fabs from E. coli. Libraries containing all possible amino acid mutations (except Cysteine) for a single CDR amino acid position were transformed into E. coli and plated on antibiotic containing Agar plates. E. coli colonies were picked and grown in 96 deep well blocks. Crude supernatants containing secreted Fabs were screened for their ability to bind TCRβV6-5 antigen by ELISA. Clones with improved binding over wildtype Fab produced in the same way were selected for sequencing. The Fab mutants with strengthened ELISA binding signal to the TCRβV6-5 antigen were purified by affinity chromatography. Enhanced binding strength of the purified Fabs to the TCRβV6-5 antigen was confirmed by affinity ranking using a Biacore instrument.

Human TCRβV6-5-binding results from the ELISA are provided in Table 13 and Table 14 for the TCRβV6-5 Fabs. Results of average OD of the library for the VL mutants is provided in FIG. 17A while results for the VH mutants is provided in FIG. 17B. The tolerance of a single pointed mutation is determined by average OD of substitution AA/WT AA with a cutoff value of 0.34. A single pointed mutation with a value higher than 0.34 is determined to be tolerated.

Human TCRβV6-5-binding results from Biacore are provided in Table 15 and Table 16 for the TCRβV6-5 Fabs.

### B. Combinatorial Library Design and Screening

TCRβV6-5-binding domains were affinity matured based on the sequence activity relationships established from the single CDR mutations identified in the VH and VL sequences that strengthened binding to the TRBV antigen. Mutations were selected to be combined into a combinatorial optimization library. The combinatorial library was constructed with random combinations of identified mutations by polymerase chain reaction (PCR). A combinatorial mutant library was established. Table 11 below displays the framework of the library. Table 12 below provides exemplary sequences for TCRβV6-5-binding domains.

TABLE 11
Combinatorial Mutant Library Design

| | VH | | | | | VL | | | |
|---|---|---|---|---|---|---|---|---|---|
| CDR position | 31 | 32 | 54 | 98 | 103 | 32 | 56 | 93 | 96 |
| Wildtype Amino acid | T | Y | G | G | Y | N | S | S | L |
| Variable mutations | L | W | R | A | F | R | K | F | I |
| | | | | | | | R | | |

TABLE 12
Exemplary Sequences for TCRβV6-5-binding Domains

| SEQ | | |
|---|---|---|
| ID | Des- | |
| NO | cription | Sequence |
| 74 | VH CDR1 | G-X₀-X₁-X₂-X₃-X₄-X₅-Y-I-H |
| | Consensus | X₀: Y, E, H, P |
| | | X₁: S, A, N, Q, E, G, H, I, L, K, M, P, T, V |
| | | X₂: F, I |
| | | X₃: T, A, R, N, Q, E, G, H, I, L, K, M, |
| | | F, S, W, Y, V |
| | | X₄: T, Q, H, L, F, S, Y |
| | | X₅: Y, A, G, I, L, M, F, T, W, V |
| Not | VH CDR1 | X₃₁X₃₂Y₃₃I₃₄H₃₅ |
| Appli- | Kabat | X₃₁: T, L, H, Y, Q, K, F, S, A, D, V, I, |
| cable | | R, W, E, G, N, P |
| | | X₃₂: Y, F, M, W, L, R, T, G, I, V, A, |
| | | E, Q, P, H, D, N, K |
| Not | VH CDR1 | G₂₆-X₂₇-X₂₈-X₂₉-X₃₀-X₃₁-X₃₂-Y₃₃ |
| Appli- | IMGT | X₂₇: Y, P, E, H, V, K, S, Q, T, L, N, |
| cable | | F, M, I, R, G, W, D |
| | | X₂₈: S, K, A, P, T, V, N, Q, L, E, I, H, |
| | | M, G, Y, W |
| | | X₂₉: F, I, M, Q, L, W, P, V, S, Y, |
| | | N, A, D, T, E, H, K, G, R |
| | | X₃₀: T, V, L, Y, W, Q, I, E, R, G, N, |
| | | F, K, M, S, H, A, P |
| | | X₃₁: T, L, H, Y, Q, K, F, S, A, D, V, |
| | | I, R, W, E, G, N, P |
| | | X₃₂: Y, F, M, W, L, R, T, G, I, V, A, |
| | | E, Q, P, H, D, N, K |
| 75 | VH CDR2 | X₆-X₇-X₈-P-X₉-S-X₁₀-X₁₁-X₁₂-X₁₃- |
| | Consensus | Y-X₁₄-X₁₅-K-F-K-G |
| | | X₆: W, R, D |
| | | X₇: F, A, R, N, Q, E, G, H, L, K, M, T, Y, V |
| | | X₈: F, R, S, Y |
| | | X₉: G, R, Q, H, I, L, K, M, F, S, W, Y |
| | | X₁₀: G, A, R, N, D, Q, E, H, L, K, |
| | | M, S, T, W, Y |
| | | X₁₁: N, R, K |
| | | X₁₂: I, A, R, N, D, Q, E, L, K, M, |
| | | F, P, S, T, W, Y, V |
| | | X₁₃: K, A, N, Q, E, H, L, M, F, S, T, W, V |
| | | X₁₄: N, D, Q, E, G, H, I, K, M, F, |
| | | P, S, T, W, Y, V |
| | | X₁₅: E, A, R, N, D, Q, G, H, I, L, K, M, |
| | | F, P, S, T, W, Y, V |
| Not | VH CDR2 | X₅₀X₅₁X₅₂X₅₃X₅₄X₅₅X₅₆X₅₇X₅₈X₅₉Y₆₀- |
| Appli- | Kabat | X₆₁X₆₂K₆₃F₆₄K₆₅G₆₆ |
| cable | | X₅₀: W, E, D, R, V, H, G, Q, S, M, N, |
| | | Y, I, L, A, T, P, F |
| | | X₅₁: F, E, H, M, K, Q, Y, L, N, V, T, |
| | | A, R, G, S, D, W, I, P |
| | | X₅₂: F, Y, R, S, A, K, W, N, M, H, |
| | | G, Q, D, P, V, L, E, T, I |
| | | X₅₃: P, G, N, A, I, V, L, T, Y, S, Q, |
| | | K, F, R, D, E, W |
| | | X₅₄: G, R, K, W, L, F, Y, Q, H, I, M, |
| | | S, D, P, T, V |
| | | X₅₅: S, T, G, W, H, N, Y, L, A, F, Q, |
| | | K, V, M, D, E, R, P |
| | | X₅₆: G, Q, R, S, W, A, H, M, N, Y, E, |
| | | L, T, D, K, I, V, P |
| | | X₅₇: N, K, R, T, G, H, V, Y, M, L, Q, I, |
| | | D, A, E, F, W, S, P |
| | | X₅₈: I, V, Y, L, A, Q, K, T, M, E, |
| | | S, N, R, F, D, P, W |
| | | X₅₉: K, N, Q, M, L, H, F, S, A, V, |
| | | T, W, E, G, P, D, R |
| | | X₆₁: N, V, S, H, P, T, D, E, W, G, |
| | | M, Q, F, K, I, Y, L |
| | | X₆₂: E, P, Q, I, N, W, H, F, K, A, V, |
| | | Y, M, L, S, D, R, T, G |
| Not | VH CDR2 | X₅₁X₅₂X₅₃X₅₄X₅₅X₅₆X₅₇X₅₈ |
| Appli- | IMGT | X₅₁: F, E, H, M, K, Q, Y, L, N, V, T, |
| cable | | A, R, G, S, D, W, I, P |
| | | X₅₂: F, Y, R, S, A, K, W, N, M, H, |
| | | G, Q, D, P, V, L, E, T, I |
| | | X₅₃: P, G, N, A, I, V, L, T, Y, S, Q, |
| | | K, F, R, D, E, W |
| | | X₅₄: G, R, K, W, L, F, Y, Q, H, I, |
| | | M, S, D, P, T, V |
| | | X₅₅: S, T, G, W, H, N, Y, L, A, F, Q, |
| | | K, V, M, D, E, R, P |
| | | X₅₆: G, Q, R, S, W, A, H, M, N, Y, |
| | | E, L, T, D, K, I, V, P |
| | | X₅₇: N, K, R, T, G, H, V, Y, M, L, |
| | | Q, I, D, A, E, F, W, S, P |
| | | X₅₈: I, V, Y, L, A, Q, K, T, M, E, |
| | | S, N, R, F, D, P, W |
| 76 | VH CDR3 | A-X₁₆-X₁₇-Y-X₁₈-X₁₉-X₂₀-X₂₁-V-X₂₂-D-X₂₃ |
| | Consensus | X₁₆: G, A |
| | | X₁₇: S, G and A |
| | | X₁₈: Y, W |
| | | X₁₉: S, A, T |
| | | X₂₀: Y, F |
| | | X₂₁: D, N |
| | | X₂₂: L, I, M |
| | | X₂₃: Y, W |
| Not | VH CDR3 | X₉₉X₁₀₀X₁₀₁X₁₀₂X₁₀₃X₁₀₄X₁₀₅X₁₀₆X₁₀₇X₁₀₈ |
| Appli- | Kabat | X₉₉: S, A, P, K, D, E, H, G, L, W, V, |
| cable | | Y, I, N, M, R, F |
| | | X₁₀₀: Y, V, F, I, L, M, T, W, H, D, Q, |
| | | A, S, E, K, P, N, R |
| | | X₁₀₁: Y, W, F, V, H, M, P, Q, S, |
| | | T, E, N, L, K, D, I, A |
| | | X₁₀₂: S, A, T, G, N, L, M, V, Y, |
| | | H, Q, W, I, D, R, E, F |
| | | X₁₀₃: Y, F, P, M, K, W, S, V, H, |
| | | L, R, D, A, E, I, Q, N |
| | | X₁₀₄: D, N, S, G, L, T, K, Q, H, |
| | | P, R, E, W, F, V |
| | | X₁₀₅: V, A, N, Q, T, W, E, P, Y, M, |
| | | K, H, D, S, F, G, L, R |
| | | X₁₀₆: L, I, M, V, Y, A, P, R, T, Q, |
| | | H, G, K, N, E, D, S |
| | | X₁₀₇: D, V, S, Q, I, H, E, R, T, M, Y, |
| | | G, N, A, F, P, K, W, L |
| | | X₁₀₈: Y, D, V, S, Q, I, H, E, R, T, M, G, |
| | | N, A, F, P, K, W, L |
| Not | VH CDR3 | A₉₇X₉₈X₉₉X₁₀₀X₁₀₁X₁₀₂X₁₀₃X₁₀₄X₁₀₅X₁₀₆X₁₀₇X₁₀₈ |
| Appli- | IMGT | X₉₈: G, A, V, P, M, I, D, Q, N, |
| cable | | F, L, W, C, and R |
| | | X₉₉: S, A, P, K, D, E, H, G, L, W, |
| | | V, Y, I, N, M, R, F |
| | | X₁₀₀: Y, V, F, I, L, M, T, W, H, D, |
| | | Q, A, S, E, K, P, N, R |
| | | X₁₀₁: Y, W, F, V, H, M, P, Q, S, T, E, N, |
| | | L, K, D, I, A |
| | | X₁₀₂: S, A, T, G, N, L, M, V, Y, H, |
| | | Q, W, I, D, R, E, F |
| | | X₁₀₃: Y, F, P, M, K, W, S, V, H, L, |
| | | R, D, A, E, I, Q, N |
| | | X₁₀₄: D, N, S, G, L, T, K, Q, H, P, |
| | | R, E, W, F, V |
| | | X₁₀₅: V, A, N, Q, T, W, E, P, Y, M, K, |
| | | H, D, S, F, G, L, R |
| | | X₁₀₆: L, I, M, V, Y, A, P, R, T, Q, |
| | | H, G, K, N, E, D, S |
| | | X₁₀₇: D, V, S, Q, I, H, E, R, T, M, Y, |
| | | G, N, A, F, P, K, W, L |
| | | X₁₀₈: Y, D, V, S, Q, I, H, E, R, T, M, |
| | | G, N, A, F, P, K, W, L |
| 77 | VL CDR1 | K-A-S-Q-Z₀-Z₁-Z₂-Z₃-Z₄-Z₅-Z₆ |
| | Consensus | Z₀: A, R, N, D, Q, E, G, H, I, L, M, |
| | | F, P, S, T, W, Y, V |
| | | Z₁: V, A, D, Q, G, H, L, K, M, F, P, S, T, W, Y |
| | | Z₂: G, A, R, N, D, Q, E, H, I, L, K, M, S, W, V |
| | | Z₃: I, A, N, Q, E, G, H, M, F, S, W, Y, V |
| | | Z₄: N, K, S |
| | | Zs: V, A, N, D, Q, E, G, L, F, S, T |
| | | Z₆: V, T |
| Not | VL CDR1 | K₂₄A₂₅S₂₆Q₂₇Z₂₈Z₂₉Z₃₀Z₃₁Z₃₂Z₃₃Z₃₄ |
| Appli- | Kabat | Z₂₈: N, H, P, T, S, M, G, A, V, W, D, |
| cable | | I, F, R, Y, Q, E, L |
| | | Z₂₉: V, G, A, L, M, S, P, F, Q, T, D, K, |
| | | W, Y, H, R, E |
| | | Z₃₀: G, W, A, L, Q, N, R, M, P, D, |
| | | K, H, I, V, S, E, F |
| | | Z₃₁: I, E, Q, G, H, S, W, Y, M, N, |
| | | P, F, A, V, K, D, L |
| | | Z₃₂: N, S, H, A, K, Q, L, D, M, T, |
| | | E, Y, V, F, W, R, P |
| | | Z₃₃: V, D, E, N, L, G, A, T, S, Q, H, |
| | | F, R, K, W, P, Y |
| | | Z₃₄: V, T, N, I, K, P, H, M, F, Q, G, |
| | | Y, R, W, L, D, A, S |
| Not | VL CDR1 | Q₂₇Z₂₈Z₂₉Z₃₀Z₃₁Z₃₂ |
| Appli- | IMGT | Z₂₈: N, H, P, T, S, M, G, A, V, W, D, |
| cable | | I, F, R, Y, Q, E, L |
| | | Z₂₉: V, G, A, L, M, S, P, F, Q, T, D, |
| | | K, W, Y, H, R, E |
| | | Z₃₀: G, W, A, L, Q, N, R, M, P, D, |
| | | K, H, I, V, S, E, F |
| | | Z₃₁: I, E, Q, G, H, S, W, Y, M, N, |
| | | P, F, A, V, K, D, L |
| | | Z₃₂: N, S, H, A, K, Q, L, D, M, T, E, |
| | | Y, V, F, W, R, P |
| 78 | VL CDR2 | Z₇-S-Z₈-H-Z₉-Z₁₀-Z₁₁ |
| | Consensus | Z₇: S, A, G, H, V |
| | | Z₈: S, N, G, M, F, T, W, Y |
| | | Z₉: R, A, N, I, L, K, P, T, V |
| | | Z₁₀: Y, W |
| | | Z₁₁: S, R, N, Q, G, H, I, K, F, T, W, Y, V |
| Not | VL CDR2 | Z₅₀S₅₁Z₅₂Z₅₃Z₅₄Z₅₅Z₅₆ |
| Appli- | Kabat | Z₅₀: S, V, A, G, H, L, T, M, F, Q, E, P, K, W, Y |
| cable | | Z₅₂: S, W, Y, G, T, M, F, N, L, A, V, |
| | | Q, I, E, D, H, P, K |
| | | Z₅₃: H, M, A, Y, L, N, E, V, G, Q, |
| | | F, K, P, W, R |
| | | Z₅₄: R, V, N, P, K, T, W, A, M, L, I, H, Q, F, D |
| | | Z₅₅: Y, W, M, F, A, S, E, G, V, P, D, |
| | | N, R, Q, T, H |
| | | Z₅₆: S, R, K, Q, Y, W, F, H, I, V, T, |
| | | C, N, G, D, MP |
| Not | VL CDR2 | Z₅₀S₅₁Z₅₂ |
| Appli- | IMGT | Z₅₀: S, V, A, G, H, L, T, M, F, Q, E, P, K, W, Y |
| cable | | Z₅₂: S, W, Y, G, T, M, F, N, L, A, |
| | | V, Q, I, E, D, H, P, K |
| 79 | VL CDR3 | Z₁₂-Z₁₃-F-Z₁₄-Z₁₅-Z₁₆-Z₁₇-Z₁₈-Z₁₉ |
| | Consensus | Z₁₂: Q, A, N, E, I, K, Y, V |
| | | Z₁₃: N, Q, E, H, M |
| | | Z₁₄: A, R, Q, G, H, I, K, M, F, S, T, W, Y, V |
| | | Z₁₅: S, A, R, N, D, Q, G, I, L, K, |
| | | M, F, T, W, V |
| | | Z₁₆: Y, A, R, N, Q, E, I, L, V |
| | | Z₁₇: A, N, D, G, H, P, S, T, V |
| | | Z₁₈: L, I, M, V |
| | | Z₁₉: A, R, N, Q, E, G, H, I, L, M, F, S, T, W, V |
| Not | VL CDR3 | Z₈₉Z₉₀Z₉₁Z₉₂Z₉₃Z₉₄Z₉₅Z₉₆Z₉₇ |
| Appli- | Kabat | Z₈₉: Q, Y, K, E, V, A, N, I, G, W, S, R, L, H |
| cable | | Z₉₀: Q, M, H, E, N, L, D, G, V, R, |
| | | I, Y, W, K, SP |
| | | Z₉₁: F, S, T, A, M, H, L, E, Y, W, N, R, |
| | | Q, I, P, V, G, K, D |
| | | Z₉₂: K, G, I, R, S, V, Y, F, W, M, A, Q, |
| | | T, H, E, L, D, N, P |
| | | Z₉₃: S, F, R, A, L, M, K, D, W, N, V, Q, T, I, G |
| | | Z₉₄: Y, L, V, I, A, N, Q, R, E, G, P, |
| | | S, K, D, W, F |
| | | Z₉₅: P, N, A, G, S, H, V, D, K, T, |
| | | L, E, M, Q, R, F, W |
| | | Z₉₆: L, I, V, M, E, Y, F, N, A, G, |
| | | H, R, S, W, Q, D, P, T |
| | | Z₉₇: T, G, A, S, R, Q, V, W, E, L, |
| | | I, H, M, F, N, D, P |
| | VL CDR3 | Z₈₉Z₉₀Z₉₁Z₉₂Z₉₃Z₉₄Z₉₅Z₉₆Z₉₇ |
| | IMGT | Z₈₉: Q, Y, K, E, V, A, N, I, G, W, S, R, L, H |
| | | Z₉₀: Q, M, H, E, N, L, D, G, V, |
| | | R, I, Y, W, K, SP |
| | | Z₉₁: F, S, T, A, M, H, L, E, Y, W, N, |
| | | R, Q, I, P, V, G, K, D |
| | | Z₉₂: K, G, I, R, S, V, Y, F, W, M, A, |
| | | Q, T, H, E, L, D, N, P |
| | | Z₉₃: S, F, R, A, L, M, K, D, W, N, V, Q, T, I, G |
| | | Z₉₄: Y, L, V, I, A, N, Q, R, E, G, |
| | | P, S, K, D, W, F |
| | | Z₉₅: P, N, A, G, S, H, V, D, K, T, |
| | | L, E, M, Q, R, F, W |
| | | Z₉₆: L, I, V, M, E, Y, F, N, A, G, H, |
| | | R, S, W, Q, D, P, T |
| | | Z₉₇: T, G, A, S, R, Q, V, W, E, L, I, |
| | | H, M, F, N, D, P |

The combinatorial library was transformed into E. coli, plated on antibiotic agar, colonies picked, and grown in 96 deep well blocks. Mutant Fabs were expressed and secreted into the supernatant. Crude Fab supernatants were screened by ELISA and those with stronger binding signal compared to wildtype were sequenced. Fabs were then expressed and purified by affinity chromatography before rank ordered by SPR affinity measurements using a Biacore instrument.

Employing the library in Table 11 above, the TCRβV6-5 mutants were designed and tested by ELISA. The results for combinatorial mutant binding to human TCRβV6-5 by ELISA are shown in Table 17. Results for combinatorial mutant binding to human TCRβV6-5 by Biacore are shown in Table 18. Results for combinatorial mutant binding to cynomolgus monkey PBMCs by flow cytometry are shown in Table 19.

C. Generation and Testing of Anti TCRβV6-5 Polypeptide Molecules Resulting from Mutational Analysis

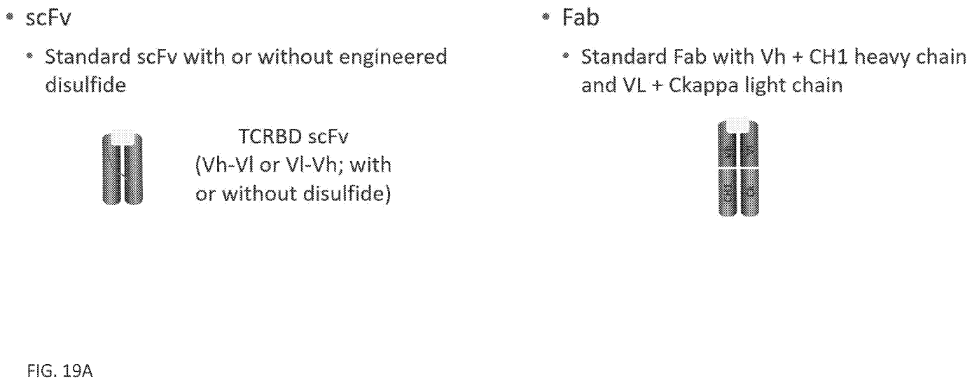

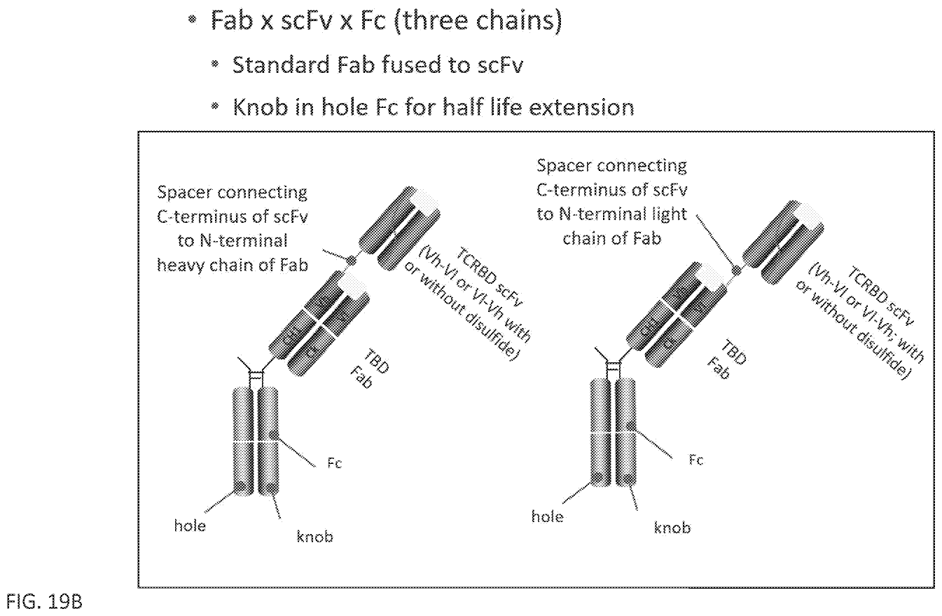

Exemplary Fabs, scFvs, and bispecific polypeptide molecules were generated to assess any impacts of reformatting the Fabs into different scFvs or bispecifics as well as assess any potential mammalian post-translational modifications of the TCRβV6-5-binding domains that may not be present in bacterially expressed binding domains of the same sequence. FIG. 19A shows exemplary formats for scFv and Fab binding domains of the disclosure. FIG. 19B shows exemplary formats for bispecific polypeptide molecules. Table 20 shows exemplary TCRβV6-5-binding domains. Table 21 shows exemplary TCRβV6-5 scFvs. Table 22 shows exemplary TCRβV6-5 Fabs.

Briefly, CHO cells were transfected with appropriate expression vectors via electroporation, allowed to recover, and then incubated in shake flasks in batch mode. Cells were fed on Day 3 and Day 5 of the culture before harvesting the conditioned medium at day 7. Conditioned medium containing the crude Fabs were purified by affinity chromatography followed by ion exchange polishing. Purified proteins of interest produced in CHO cells were then tested in ELISAs, cell based binding studies, and functional assays.

Polypeptide molecules were evaluated for their ability to bind the indicated human or cynomolgus monkey TCRβV6-5 antigens by ELISA. Briefly, soluble antigen proteins were either directly coated on high-binding ELISA plates or biotinylated antigens were captured on neutravidin coated plates. A polypeptide molecule diluted in buffer was then added to the antigen coated plates. Bound polypeptide molecule was detected using a standard horse radish peroxidase conjugate secondary antibody. The concentration of polypeptide molecule required to achieve 50% maximal signal (EC50) was calculated using Graphpad Prism software.

ELISA binding results are displayed at FIGS. 20A-20F. Results at FIGS. 20A-20C show that affinity maturation of the original T cell binder strengthened human TCRβV6-5-binding affinity by 2-fold (FIG. 20A), but drastically strengthened increased cyno TRBV6-2 binding greater than 800-fold (FIG. 20B). A summary of all EC results is displayed at FIG. 20C. Results show equivalent human and cyno antigen binding affinity for some TCRβV6-5 polypeptide molecules.

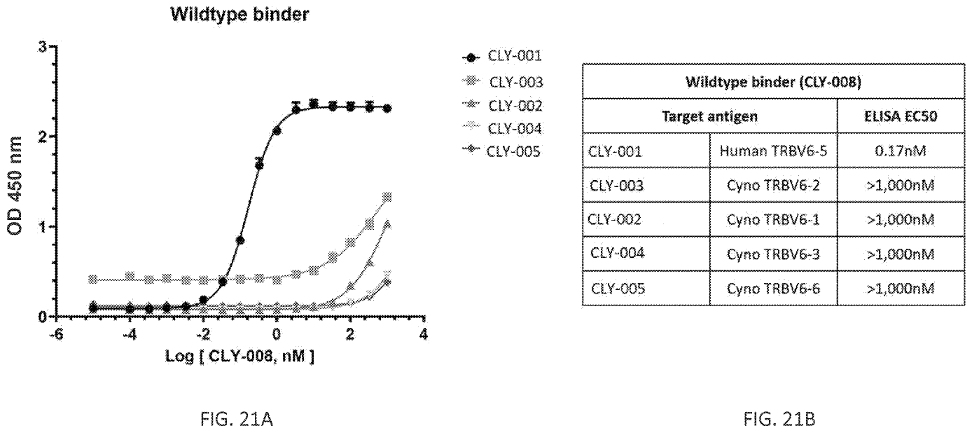

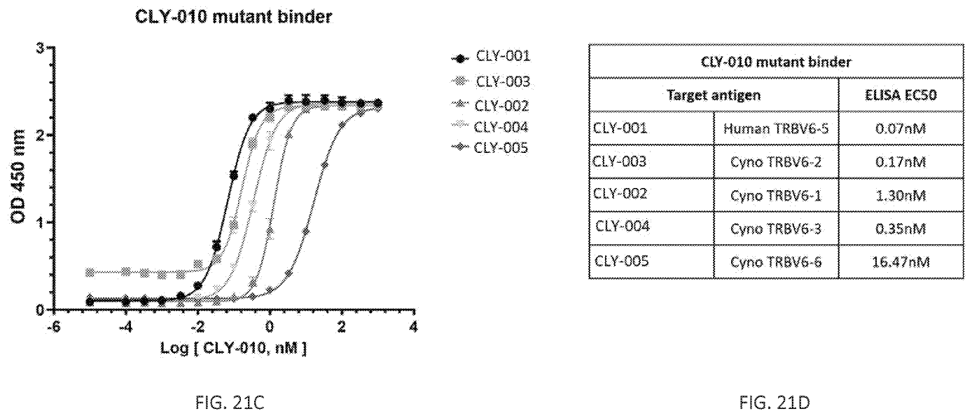

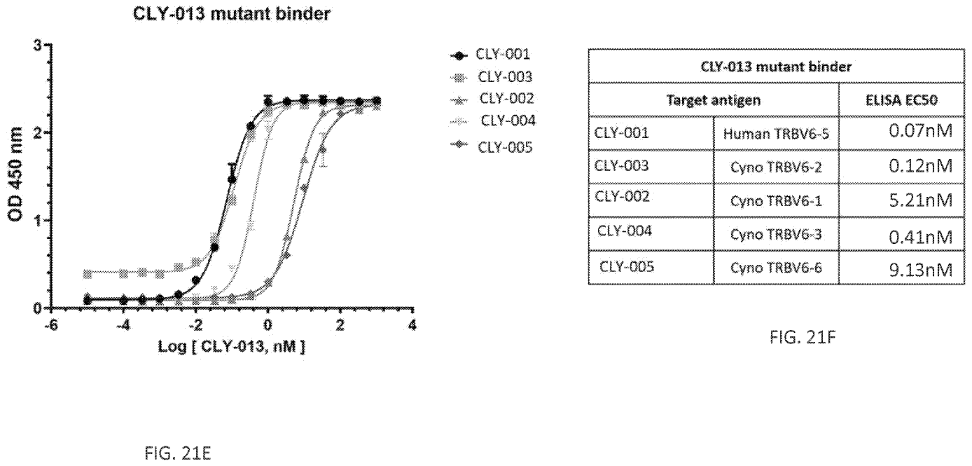

Additional ELISA binding results displayed at FIGS. 21A-21F show that as compared to wildtype polypeptide molecules (FIG. 21A-FIG. 21B), mutant polypeptide molecules CLY-010 (FIG. 21C-FIG. 21D) and CLY-013 (FIG. 21E-FIG. 21F), exhibited superior binding to human TCRβV6-5, cyno TCRβV6-2, cyno TCRβV6-1, cyno-TRβV6-3, or cyno-TCRβV6-6.

TABLE 13
Light Chain Mutant Fab Binding to Human TCRβV6-5 by ELISA

| | Human TCRβV6-5-binding ELISA | | | | | | | |
|---|---|---|---|---|---|---|---|---|
| | Mutation | WT | Ratio = | Mutations in VL CDRs | | | SEQ ID NOS | |
| | OD | OD | OD(Mutation)/ | | | Point | Fab | Fab |
| ID | 450 nm | 450 nm | OD(WT) | Chain | CDR | mutation | LC | HC |
| AHF37613 | 0.540 | 0.783 | 0.689 | VL | CDRL1 | N28A | 245 | 576 |
| AHF37604 | 0.516 | 0.783 | 0.659 | VL | CDRL1 | N28D | 246 | 576 |
| AHF37585 | 0.434 | 0.783 | 0.554 | VL | CDRL1 | N28E | 247 | 576 |
| AHF37607 | 0.498 | 0.783 | 0.636 | VL | CDRL1 | N28F | 248 | 576 |
| AHF37614 | 0.551 | 0.783 | 0.704 | VL | CDRL1 | N28G | 249 | 576 |
| AHF37623 | 0.628 | 0.783 | 0.802 | VL | CDRL1 | N28H | 250 | 576 |
| AHF37597 | 0.510 | 0.783 | 0.652 | VL | CDRL1 | N28I | 251 | 576 |
| AHF37588 | 0.426 | 0.783 | 0.543 | VL | CDRL1 | N28L | 252 | 576 |
| AHF37618 | 0.559 | 0.783 | 0.714 | VL | CDRL1 | N28M | 253 | 576 |
| AHF37628 | 0.654 | 0.783 | 0.835 | VL | CDRL1 | N28N | 231 | 576 |
| AHF37619 | 0.589 | 0.783 | 0.752 | VL | CDRL1 | N28P | 254 | 576 |
| AHF37587 | 0.462 | 0.783 | 0.590 | VL | CDRL1 | N28Q | 255 | 576 |
| AHF37596 | 0.495 | 0.783 | 0.633 | VL | CDRL1 | N28R | 256 | 576 |
| AHF37621 | 0.560 | 0.783 | 0.715 | VL | CDRL1 | N28S | 257 | 576 |
| AHF37615 | 0.561 | 0.783 | 0.716 | VL | CDRL1 | N28T | 258 | 576 |
| AHF37606 | 0.537 | 0.783 | 0.686 | VL | CDRL1 | N28V | 259 | 576 |
| AHF37612 | 0.534 | 0.783 | 0.682 | VL | CDRL1 | N28W | 260 | 576 |
| AHF37591 | 0.491 | 0.783 | 0.627 | VL | CDRL1 | N28Y | 261 | 576 |
| AHF37641 | 0.755 | 0.783 | 0.964 | VL | CDRL1 | V29A | 262 | 576 |
| AHF37636 | 0.535 | 0.783 | 0.684 | VL | CDRL1 | V29D | 263 | 576 |
| AHF37655 | 0.211 | 0.783 | 0.270 | VL | CDRL1 | V29E | 264 | 576 |
| AHF37669 | 0.632 | 0.783 | 0.807 | VL | CDRL1 | V29F | 265 | 576 |
| AHF37672 | 0.799 | 0.783 | 1.020 | VL | CDRL1 | V29G | 266 | 576 |
| AHF37654 | 0.332 | 0.783 | 0.424 | VL | CDRL1 | V29H | 267 | 576 |
| AHF37656 | 0.470 | 0.783 | 0.600 | VL | CDRL1 | V29K | 268 | 576 |
| AHF37643 | 0.753 | 0.783 | 0.961 | VL | CDRL1 | V29L | 269 | 576 |
| AHF37658 | 0.749 | 0.783 | 0.957 | VL | CDRL1 | V29M | 270 | 576 |
| AHF37635 | 0.697 | 0.783 | 0.890 | VL | CDRL1 | V29P | 271 | 576 |
| AHF37671 | 0.625 | 0.783 | 0.798 | VL | CDRL1 | V29Q | 272 | 576 |
| AHF37659 | 0.748 | 0.783 | 0.955 | VL | CDRL1 | V29S | 273 | 576 |
| AHF37634 | 0.625 | 0.783 | 0.798 | VL | CDRL1 | V29T | 274 | 576 |
| AHF37653 | 0.369 | 0.783 | 0.471 | VL | CDRL1 | V29W | 1085 | 576 |
| AHF37668 | 0.361 | 0.783 | 0.461 | VL | CDRL1 | V29Y | 1086 | 576 |
| AHF37719 | 0.507 | 0.655 | 0.774 | VL | CDRL1 | G30A | 1087 | 576 |
| AHF37675 | 0.379 | 0.655 | 0.578 | VL | CDRL1 | G30D | 1088 | 576 |
| AHF37673 | 0.280 | 0.655 | 0.427 | VL | CDRL1 | G30E | 1089 | 576 |
| AHF37680 | 0.172 | 0.655 | 0.263 | VL | CDRL1 | G30F | 1090 | 576 |
| AHF37704 | 0.358 | 0.655 | 0.546 | VL | CDRL1 | G30H | 1091 | 576 |
| AHF37703 | 0.338 | 0.655 | 0.516 | VL | CDRL1 | G30I | 1092 | 576 |
| AHF37702 | 0.378 | 0.655 | 0.576 | VL | CDRL1 | G30K | 1093 | 576 |
| AHF37706 | 0.506 | 0.655 | 0.771 | VL | CDRL1 | G30L | 275 | 576 |
| AHF37705 | 0.407 | 0.655 | 0.622 | VL | CDRL1 | G30M | 276 | 576 |
| AHF37708 | 0.460 | 0.655 | 0.702 | VL | CDRL1 | G30N | 277 | 576 |
| AHF37697 | 0.392 | 0.655 | 0.598 | VL | CDRL1 | G30P | 278 | 576 |
| AHF37714 | 0.477 | 0.655 | 0.727 | VL | CDRL1 | G30Q | 279 | 576 |
| AHF37694 | 0.428 | 0.655 | 0.653 | VL | CDRL1 | G30R | 280 | 576 |
| AHF37681 | 0.313 | 0.655 | 0.478 | VL | CDRL1 | G30S | 281 | 576 |
| AHF37677 | 0.336 | 0.655 | 0.513 | VL | CDRL1 | G30V | 282 | 576 |
| AHF37718 | 0.543 | 0.655 | 0.828 | VL | CDRL1 | G30W | 283 | 576 |
| AHF37754 | 0.272 | 0.655 | 0.414 | VL | CDRL1 | I31A | 284 | 576 |
| AHF37761 | 0.411 | 0.655 | 0.626 | VL | CDRL1 | I31E | 285 | 576 |
| AHF37730 | 0.279 | 0.655 | 0.425 | VL | CDRL1 | I31F | 286 | 576 |
| AHF37746 | 0.382 | 0.655 | 0.582 | VL | CDRL1 | I31G | 287 | 576 |
| AHF37747 | 0.370 | 0.655 | 0.565 | VL | CDRL1 | I31H | 288 | 576 |
| AHF37741 | 0.184 | 0.655 | 0.281 | VL | CDRL1 | I31K | 289 | 576 |
| AHF37759 | 0.153 | 0.655 | 0.234 | VL | CDRL1 | I31L | 290 | 576 |
| AHF37758 | 0.293 | 0.655 | 0.447 | VL | CDRL1 | I31M | 291 | 576 |
| AHF37729 | 0.283 | 0.655 | 0.432 | VL | CDRL1 | I31N | 292 | 576 |
| AHF37737 | 0.282 | 0.655 | 0.430 | VL | CDRL1 | I31P | 293 | 576 |
| AHF37755 | 0.401 | 0.655 | 0.612 | VL | CDRL1 | I31Q | 294 | 576 |
| AHF37752 | 0.362 | 0.655 | 0.552 | VL | CDRL1 | I31S | 295 | 576 |
| AHF37734 | 0.263 | 0.655 | 0.402 | VL | CDRL1 | I31V | 296 | 576 |
| AHF37762 | 0.323 | 0.655 | 0.493 | VL | CDRL1 | I31W | 297 | 576 |
| AHF37739 | 0.309 | 0.655 | 0.472 | VL | CDRL1 | I31Y | 298 | 576 |
| AHF37798 | 0.271 | 0.737 | 0.368 | VL | CDRL1 | N32A | 299 | 576 |
| AHF37765 | 0.113 | 0.737 | 0.153 | VL | CDRL1 | N32D | 300 | 576 |
| AHF37806 | 0.072 | 0.737 | 0.098 | VL | CDRL1 | N32E | 301 | 576 |
| AHF37811 | 0.062 | 0.737 | 0.084 | VL | CDRL1 | N32F | 302 | 576 |
| AHF37791 | 0.301 | 0.737 | 0.409 | VL | CDRL1 | N32H | 303 | 576 |
| AHF37768 | 0.270 | 0.737 | 0.367 | VL | CDRL1 | N32K | 304 | 576 |
| AHF37792 | 0.181 | 0.737 | 0.245 | VL | CDRL1 | N32L | 305 | 576 |
| AHF37805 | 0.104 | 0.737 | 0.141 | VL | CDRL1 | N32M | 306 | 576 |
| AHF37782 | 0.058 | 0.737 | 0.079 | VL | CDRL1 | N32P | 307 | 576 |
| AHF37812 | 0.230 | 0.737 | 0.312 | VL | CDRL1 | N32Q | 308 | 576 |
| AHF37773 | 0.340 | 0.737 | 0.461 | VL | CDRL1 | N32S | 309 | 576 |
| AHF37777 | 0.089 | 0.737 | 0.120 | VL | CDRL1 | N32T | 310 | 576 |
| AHF37797 | 0.063 | 0.737 | 0.085 | VL | CDRL1 | N32V | 311 | 576 |
| AHF37803 | 0.061 | 0.737 | 0.082 | VL | CDRL1 | N32W | 312 | 576 |
| AHF37778 | 0.072 | 0.737 | 0.098 | VL | CDRL1 | N32Y | 313 | 576 |
| AHF37825 | 0.623 | 0.737 | 0.845 | VL | CDRL1 | V33A | 314 | 576 |
| AHF37847 | 0.788 | 0.737 | 1.069 | VL | CDRL1 | V33D | 315 | 576 |
| AHF37838 | 0.692 | 0.737 | 0.938 | VL | CDRL1 | V33E | 316 | 576 |
| AHF37822 | 0.334 | 0.737 | 0.453 | VL | CDRL1 | V33F | 317 | 576 |
| AHF37837 | 0.634 | 0.737 | 0.860 | VL | CDRL1 | V33G | 318 | 576 |
| AHF37841 | 0.412 | 0.737 | 0.558 | VL | CDRL1 | V33H | 319 | 576 |
| AHF37814 | 0.155 | 0.737 | 0.210 | VL | CDRL1 | V33K | 320 | 576 |
| AHF37823 | 0.669 | 0.737 | 0.907 | VL | CDRL1 | V33L | 321 | 576 |
| AHF37850 | 0.689 | 0.737 | 0.935 | VL | CDRL1 | V33N | 322 | 576 |
| AHF37852 | 0.056 | 0.737 | 0.076 | VL | CDRL1 | V33P | 323 | 576 |
| AHF37854 | 0.567 | 0.737 | 0.769 | VL | CDRL1 | V33Q | 324 | 576 |
| AHF37824 | 0.260 | 0.737 | 0.353 | VL | CDRL1 | V33R | 325 | 576 |
| AHF37817 | 0.575 | 0.737 | 0.780 | VL | CDRL1 | V33S | 326 | 576 |
| AHF37836 | 0.584 | 0.737 | 0.791 | VL | CDRL1 | V33T | 327 | 576 |
| AHF37855 | 0.058 | 0.737 | 0.079 | VL | CDRL1 | V33W | 328 | 576 |
| AHF37832 | 0.047 | 0.737 | 0.064 | VL | CDRL1 | V33Y | 329 | 576 |
| AHF37858 | 0.049 | 0.711 | 0.069 | VL | CDRL1 | V34D | 330 | 576 |
| AHF37887 | 0.059 | 0.711 | 0.083 | VL | CDRL1 | V34F | 331 | 576 |
| AHF37879 | 0.055 | 0.711 | 0.077 | VL | CDRL1 | V34G | 332 | 576 |
| AHF37870 | 0.068 | 0.711 | 0.096 | VL | CDRL1 | V34H | 333 | 576 |
| AHF37904 | 0.093 | 0.711 | 0.131 | VL | CDRL1 | V34I | 334 | 576 |
| AHF37865 | 0.080 | 0.711 | 0.113 | VL | CDRL1 | V34K | 335 | 576 |
| AHF37867 | 0.050 | 0.711 | 0.071 | VL | CDRL1 | V34L | 336 | 576 |
| AHF37874 | 0.063 | 0.711 | 0.089 | VL | CDRL1 | V34M | 1094 | 576 |
| AHF37897 | 0.099 | 0.711 | 0.139 | VL | CDRL1 | V34N | 337 | 576 |
| AHF37857 | 0.073 | 0.711 | 0.103 | VL | CDRL1 | V34P | 338 | 576 |
| AHF37893 | 0.058 | 0.711 | 0.081 | VL | CDRL1 | V34Q | 339 | 576 |
| AHF37895 | 0.051 | 0.711 | 0.072 | VL | CDRL1 | V34R | 340 | 576 |
| AHF37866 | 0.048 | 0.711 | 0.067 | VL | CDRL1 | V34S | 341 | 576 |
| AHF37903 | 0.307 | 0.711 | 0.432 | VL | CDRL1 | V34T | 342 | 576 |
| AHF37896 | 0.051 | 0.711 | 0.072 | VL | CDRL1 | V34W | 343 | 576 |
| AHF37862 | 0.055 | 0.711 | 0.077 | VL | CDRL1 | V34Y | 344 | 576 |
| AHF37937 | 0.583 | 0.711 | 0.820 | VL | CDRL2 | S50A | 345 | 576 |
| AHF37936 | 0.062 | 0.711 | 0.088 | VL | CDRL2 | S50E | 346 | 576 |
| AHF37946 | 0.079 | 0.711 | 0.112 | VL | CDRL2 | S50F | 347 | 576 |
| AHF37932 | 0.362 | 0.711 | 0.510 | VL | CDRL2 | S50G | 348 | 576 |
| AHF37944 | 0.276 | 0.711 | 0.389 | VL | CDRL2 | S50H | 349 | 576 |
| AHF37913 | 0.059 | 0.711 | 0.083 | VL | CDRL2 | S50K | 350 | 576 |
| AHF37929 | 0.140 | 0.711 | 0.196 | VL | CDRL2 | S50L | 351 | 576 |
| AHF37948 | 0.123 | 0.711 | 0.173 | VL | CDRL2 | S50M | 352 | 576 |
| AHF37928 | 0.063 | 0.711 | 0.088 | VL | CDRL2 | S50P | 353 | 576 |
| AHF37920 | 0.070 | 0.711 | 0.099 | VL | CDRL2 | S50Q | 354 | 576 |
| AHF37945 | 0.137 | 0.711 | 0.193 | VL | CDRL2 | S50T | 355 | 576 |
| AHF37917 | 0.612 | 0.711 | 0.861 | VL | CDRL2 | S50V | 356 | 576 |
| AHF37907 | 0.057 | 0.711 | 0.080 | VL | CDRL2 | S50W | 357 | 576 |
| AHF37914 | 0.048 | 0.711 | 0.068 | VL | CDRL2 | S50Y | 358 | 576 |
| AHF37972 | 0.160 | 0.733 | 0.218 | VL | CDRL2 | S52A | 359 | 576 |
| AHF37961 | 0.126 | 0.733 | 0.172 | VL | CDRL2 | S52D | 360 | 576 |
| AHF37986 | 0.146 | 0.733 | 0.200 | VL | CDRL2 | S52E | 361 | 576 |
| AHF37970 | 0.371 | 0.733 | 0.506 | VL | CDRL2 | S52F | 362 | 576 |
| AHF37984 | 0.474 | 0.733 | 0.647 | VL | CDRL2 | S52G | 363 | 576 |
| AHF37957 | 0.126 | 0.733 | 0.172 | VL | CDRL2 | S52H | 364 | 576 |
| AHF37973 | 0.149 | 0.733 | 0.203 | VL | CDRL2 | S52I | 365 | 576 |
| AHF37968 | 0.060 | 0.733 | 0.082 | VL | CDRL2 | S52K | 366 | 576 |
| AHF37979 | 0.255 | 0.733 | 0.348 | VL | CDRL2 | S52L | 367 | 576 |
| AHF37980 | 0.379 | 0.733 | 0.518 | VL | CDRL2 | S52M | 368 | 576 |
| AHF37955 | 0.298 | 0.733 | 0.407 | VL | CDRL2 | S52N | 369 | 576 |
| AHF37992 | 0.083 | 0.733 | 0.114 | VL | CDRL2 | S52P | 370 | 576 |
| AHF37988 | 0.157 | 0.733 | 0.214 | VL | CDRL2 | S52Q | 371 | 576 |
| AHF37985 | 0.441 | 0.733 | 0.602 | VL | CDRL2 | S52T | 372 | 576 |
| AHF37990 | 0.158 | 0.733 | 0.216 | VL | CDRL2 | S52V | 373 | 576 |
| AHF37994 | 0.605 | 0.733 | 0.826 | VL | CDRL2 | S52W | 374 | 576 |
| AHF37987 | 0.523 | 0.733 | 0.713 | VL | CDRL2 | S52Y | 375 | 576 |
| AHF38022 | 0.219 | 0.733 | 0.299 | VL | CDRL2 | H53A | 376 | 576 |
| AHF38037 | 0.167 | 0.733 | 0.228 | VL | CDRL2 | H53E | 377 | 576 |
| AHF38031 | 0.093 | 0.733 | 0.128 | VL | CDRL2 | H53F | 378 | 576 |
| AHF37998 | 0.150 | 0.733 | 0.205 | VL | CDRL2 | H53G | 379 | 576 |
| AHF38035 | 0.063 | 0.733 | 0.086 | VL | CDRL2 | H53K | 380 | 576 |
| AHF38034 | 0.182 | 0.733 | 0.248 | VL | CDRL2 | H53L | 381 | 576 |
| AHF38006 | 0.251 | 0.733 | 0.342 | VL | CDRL2 | H53M | 382 | 576 |
| AHF38039 | 0.171 | 0.733 | 0.234 | VL | CDRL2 | H53N | 383 | 576 |
| AHF38004 | 0.056 | 0.733 | 0.077 | VL | CDRL2 | H53P | 384 | 576 |
| AHF38003 | 0.127 | 0.733 | 0.173 | VL | CDRL2 | H53Q | 385 | 576 |
| AHF38028 | 0.053 | 0.733 | 0.072 | VL | CDRL2 | H53R | 386 | 576 |
| AHF38016 | 0.157 | 0.733 | 0.215 | VL | CDRL2 | H53V | 387 | 576 |
| AHF38005 | 0.054 | 0.733 | 0.074 | VL | CDRL2 | H53W | 388 | 576 |
| AHF38013 | 0.198 | 0.733 | 0.270 | VL | CDRL2 | H53Y | 389 | 576 |
| AHF38057 | 0.407 | 0.853 | 0.477 | VL | CDRL2 | R54A | 390 | 576 |
| AHF38062 | 0.051 | 0.853 | 0.059 | VL | CDRL2 | R54D | 391 | 576 |
| AHF38059 | 0.159 | 0.853 | 0.187 | VL | CDRL2 | R54F | 392 | 576 |
| AHF38085 | 0.259 | 0.853 | 0.304 | VL | CDRL2 | R54H | 393 | 576 |
| AHF38044 | 0.368 | 0.853 | 0.431 | VL | CDRL2 | R54I | 394 | 576 |
| AHF38078 | 0.551 | 0.853 | 0.646 | VL | CDRL2 | R54K | 395 | 576 |
| AHF38067 | 0.375 | 0.853 | 0.440 | VL | CDRL2 | R54L | 396 | 576 |
| AHF38080 | 0.394 | 0.853 | 0.461 | VL | CDRL2 | R54M | 397 | 576 |
| AHF38077 | 0.612 | 0.853 | 0.717 | VL | CDRL2 | R54N | 398 | 576 |
| AHF38079 | 0.565 | 0.853 | 0.662 | VL | CDRL2 | R54P | 399 | 576 |
| AHF38068 | 0.223 | 0.853 | 0.262 | VL | CDRL2 | R54Q | 400 | 576 |
| AHF38076 | 0.481 | 0.853 | 0.563 | VL | CDRL2 | R54T | 401 | 576 |
| AHF38084 | 0.636 | 0.853 | 0.745 | VL | CDRL2 | R54V | 402 | 576 |
| AHF38087 | 0.410 | 0.853 | 0.481 | VL | CDRL2 | R54W | 403 | 576 |
| AHF38124 | 0.158 | 0.853 | 0.185 | VL | CDRL2 | Y55A | 404 | 576 |
| AHF38089 | 0.068 | 0.853 | 0.080 | VL | CDRL2 | Y55D | 405 | 576 |
| AHF38121 | 0.087 | 0.853 | 0.102 | VL | CDRL2 | Y55E | 406 | 576 |
| AHF38119 | 0.231 | 0.853 | 0.270 | VL | CDRL2 | Y55F | 407 | 576 |
| AHF38113 | 0.086 | 0.853 | 0.101 | VL | CDRL2 | Y55G | 408 | 576 |
| AHF38098 | 0.057 | 0.853 | 0.067 | VL | CDRL2 | Y55H | 409 | 576 |
| AHF38116 | 0.248 | 0.853 | 0.290 | VL | CDRL2 | Y55M | 410 | 576 |
| AHF38105 | 0.068 | 0.853 | 0.080 | VL | CDRL2 | Y55N | 411 | 576 |
| AHF38130 | 0.073 | 0.853 | 0.086 | VL | CDRL2 | Y55P | 412 | 576 |
| AHF38115 | 0.061 | 0.853 | 0.071 | VL | CDRL2 | Y55Q | 413 | 576 |
| AHF38093 | 0.067 | 0.853 | 0.079 | VL | CDRL2 | Y55R | 414 | 576 |
| AHF38132 | 0.089 | 0.853 | 0.104 | VL | CDRL2 | Y55S | 415 | 576 |
| AHF38123 | 0.061 | 0.853 | 0.071 | VL | CDRL2 | Y55T | 416 | 576 |
| AHF38097 | 0.081 | 0.853 | 0.095 | VL | CDRL2 | Y55V | 417 | 576 |
| AHF38131 | 0.295 | 0.853 | 0.345 | VL | CDRL2 | Y55W | 418 | 576 |
| AHF38139 | 0.374 | 0.802 | 0.466 | VL | CDRL2 | S56C | 419 | 576 |
| AHF38178 | 0.139 | 0.802 | 0.173 | VL | CDRL2 | S56D | 420 | 576 |
| AHF38168 | 0.465 | 0.802 | 0.580 | VL | CDRL2 | S56F | 421 | 576 |
| AHF38152 | 0.294 | 0.802 | 0.367 | VL | CDRL2 | S56G | 422 | 576 |
| AHF38150 | 0.461 | 0.802 | 0.575 | VL | CDRL2 | S56H | 423 | 576 |
| AHF38141 | 0.456 | 0.802 | 0.568 | VL | CDRL2 | S56I | 424 | 576 |
| AHF38156 | 0.800 | 0.802 | 0.999 | VL | CDRL2 | S56K | 425 | 576 |
| AHF38169 | 0.340 | 0.802 | 0.424 | VL | CDRL2 | S56N | 426 | 576 |
| AHF38166 | 0.057 | 0.802 | 0.071 | VL | CDRL2 | S56P | 427 | 576 |
| AHF38162 | 0.609 | 0.802 | 0.760 | VL | CDRL2 | S56Q | 428 | 576 |
| AHF38172 | 0.830 | 0.802 | 1.035 | VL | CDRL2 | S56R | 429 | 576 |
| AHF38179 | 0.397 | 0.802 | 0.495 | VL | CDRL2 | S56T | 430 | 576 |
| AHF38159 | 0.425 | 0.802 | 0.530 | VL | CDRL2 | S56V | 431 | 576 |
| AHF38151 | 0.542 | 0.802 | 0.676 | VL | CDRL2 | S56W | 432 | 576 |
| AHF38165 | 0.554 | 0.802 | 0.691 | VL | CDRL2 | S56Y | 433 | 576 |
| AHF38216 | 0.410 | 0.802 | 0.512 | VL | CDRL3 | Q89A | 434 | 576 |
| AHF38181 | 0.441 | 0.802 | 0.550 | VL | CDRL3 | Q89E | 435 | 576 |
| AHF38215 | 0.187 | 0.802 | 0.234 | VL | CDRL3 | Q89G | 436 | 576 |
| AHF38212 | 0.084 | 0.802 | 0.105 | VL | CDRL3 | Q89H | 437 | 576 |
| AHF38214 | 0.287 | 0.802 | 0.358 | VL | CDRL3 | Q89I | 438 | 576 |
| AHF38192 | 0.528 | 0.802 | 0.659 | VL | CDRL3 | Q89K | 439 | 576 |
| AHF38223 | 0.085 | 0.802 | 0.106 | VL | CDRL3 | Q89L | 440 | 576 |
| AHF38220 | 0.409 | 0.802 | 0.511 | VL | CDRL3 | Q89N | 441 | 576 |
| AHF38204 | 0.092 | 0.802 | 0.114 | VL | CDRL3 | Q89R | 442 | 576 |
| AHF38194 | 0.115 | 0.802 | 0.143 | VL | CDRL3 | Q89S | 443 | 576 |
| AHF38196 | 0.418 | 0.802 | 0.521 | VL | CDRL3 | Q89V | 444 | 576 |
| AHF38195 | 0.145 | 0.802 | 0.180 | VL | CDRL3 | Q89W | 445 | 576 |
| AHF38211 | 0.529 | 0.802 | 0.660 | VL | CDRL3 | Q89Y | 446 | 576 |
| AHF38267 | 0.182 | 0.598 | 0.304 | VL | CDRL3 | Q90D | 447 | 576 |
| AHF38248 | 0.315 | 0.598 | 0.527 | VL | CDRL3 | Q90E | 448 | 576 |
| AHF38262 | 0.167 | 0.598 | 0.280 | VL | CDRL3 | Q90G | 449 | 576 |
| AHF38259 | 0.376 | 0.598 | 0.630 | VL | CDRL3 | Q90H | 450 | 576 |
| AHF38237 | 0.096 | 0.598 | 0.161 | VL | CDRL3 | Q90I | 451 | 576 |
| AHF38263 | 0.065 | 0.598 | 0.108 | VL | CDRL3 | Q90K | 452 | 576 |
| AHF38266 | 0.210 | 0.598 | 0.351 | VL | CDRL3 | Q90L | 453 | 576 |
| AHF38261 | 0.442 | 0.598 | 0.739 | VL | CDRL3 | Q90M | 454 | 576 |
| AHF38265 | 0.313 | 0.598 | 0.523 | VL | CDRL3 | Q90N | 455 | 576 |
| AHF38239 | 0.044 | 0.598 | 0.073 | VL | CDRL3 | Q90P | 456 | 576 |
| AHF38238 | 0.331 | 0.598 | 0.555 | VL | CDRL3 | Q90Q | 231 | 576 |
| AHF38246 | 0.105 | 0.598 | 0.175 | VL | CDRL3 | Q90R | 457 | 576 |
| AHF38230 | 0.110 | 0.598 | 0.183 | VL | CDRL3 | Q90V | 458 | 576 |
| AHF38258 | 0.067 | 0.598 | 0.112 | VL | CDRL3 | Q90W | 459 | 576 |
| AHF38225 | 0.068 | 0.598 | 0.114 | VL | CDRL3 | Q90Y | 460 | 576 |
| AHF38294 | 0.072 | 0.598 | 0.120 | VL | CDRL3 | F91A | 461 | 576 |
| AHF38302 | 0.048 | 0.598 | 0.080 | VL | CDRL3 | F91D | 462 | 576 |
| AHF38288 | 0.066 | 0.598 | 0.111 | VL | CDRL3 | F91E | 463 | 576 |
| AHF38292 | 0.055 | 0.598 | 0.092 | VL | CDRL3 | F91G | 464 | 576 |
| AHF38307 | 0.071 | 0.598 | 0.118 | VL | CDRL3 | F91H | 465 | 576 |
| AHF38312 | 0.055 | 0.598 | 0.093 | VL | CDRL3 | F91I | 466 | 576 |
| AHF38297 | 0.055 | 0.598 | 0.091 | VL | CDRL3 | F91K | 467 | 576 |
| AHF38310 | 0.069 | 0.598 | 0.115 | VL | CDRL3 | F91L | 468 | 576 |
| AHF38296 | 0.071 | 0.598 | 0.119 | VL | CDRL3 | F91M | 469 | 576 |
| AHF38300 | 0.061 | 0.598 | 0.102 | VL | CDRL3 | F91N | 470 | 576 |
| AHF38313 | 0.056 | 0.598 | 0.093 | VL | CDRL3 | F91P | 471 | 576 |
| AHF38295 | 0.057 | 0.598 | 0.095 | VL | CDRL3 | F91Q | 472 | 576 |
| AHF38304 | 0.060 | 0.598 | 0.100 | VL | CDRL3 | F91R | 473 | 576 |
| AHF38314 | 0.104 | 0.598 | 0.174 | VL | CDRL3 | F91S | 474 | 576 |
| AHF38277 | 0.079 | 0.598 | 0.132 | VL | CDRL3 | F91T | 475 | 576 |
| AHF38286 | 0.056 | 0.598 | 0.093 | VL | CDRL3 | F91V | 476 | 576 |
| AHF38279 | 0.064 | 0.598 | 0.106 | VL | CDRL3 | F91W | 477 | 576 |
| AHF38273 | 0.064 | 0.598 | 0.108 | VL | CDRL3 | F91Y | 478 | 576 |
| AHF38354 | 0.312 | 0.645 | 0.483 | VL | CDRL3 | K92A | 479 | 576 |
| AHF38356 | 0.157 | 0.645 | 0.243 | VL | CDRL3 | K92D | 480 | 576 |
| AHF38349 | 0.247 | 0.645 | 0.382 | VL | CDRL3 | K92E | 481 | 576 |
| AHF38362 | 0.331 | 0.645 | 0.513 | VL | CDRL3 | K92F | 482 | 576 |
| AHF38360 | 0.469 | 0.645 | 0.727 | VL | CDRL3 | K92G | 483 | 576 |
| AHF38337 | 0.264 | 0.645 | 0.409 | VL | CDRL3 | K92H | 484 | 576 |
| AHF38357 | 0.385 | 0.645 | 0.597 | VL | CDRL3 | K92I | 485 | 576 |
| AHF38347 | 0.385 | 0.645 | 0.596 | VL | CDRL3 | K92K | 231 | 576 |
| AHF38325 | 0.181 | 0.645 | 0.281 | VL | CDRL3 | K92L | 486 | 576 |
| AHF38326 | 0.315 | 0.645 | 0.489 | VL | CDRL3 | K92M | 487 | 576 |
| AHF38317 | 0.129 | 0.645 | 0.199 | VL | CDRL3 | K92N | 488 | 576 |
| AHF38350 | 0.068 | 0.645 | 0.106 | VL | CDRL3 | K92P | 489 | 576 |
| AHF38358 | 0.285 | 0.645 | 0.442 | VL | CDRL3 | K92Q | 490 | 576 |
| AHF38335 | 0.374 | 0.645 | 0.580 | VL | CDRL3 | K92R | 491 | 576 |
| AHF38359 | 0.343 | 0.645 | 0.532 | VL | CDRL3 | K92S | 492 | 576 |
| AHF38341 | 0.268 | 0.645 | 0.415 | VL | CDRL3 | K92T | 493 | 576 |
| AHF38361 | 0.340 | 0.645 | 0.527 | VL | CDRL3 | K92V | 494 | 576 |
| AHF38345 | 0.317 | 0.645 | 0.491 | VL | CDRL3 | K92W | 495 | 576 |
| AHF38344 | 0.331 | 0.645 | 0.514 | VL | CDRL3 | K92Y | 496 | 576 |
| AHF38394 | 0.668 | 0.645 | 1.035 | VL | CDRL3 | S93A | 497 | 576 |
| AHF38383 | 0.625 | 0.645 | 0.969 | VL | CDRL3 | S93D | 498 | 576 |
| AHF38408 | 0.709 | 0.645 | 1.100 | VL | CDRL3 | S93F | 499 | 576 |
| AHF38371 | 0.489 | 0.645 | 0.758 | VL | CDRL3 | S93G | 500 | 576 |
| AHF38388 | 0.553 | 0.645 | 0.857 | VL | CDRL3 | S93I | 501 | 576 |
| AHF38397 | 0.629 | 0.645 | 0.974 | VL | CDRL3 | S93K | 502 | 576 |
| AHF38406 | 0.653 | 0.645 | 1.011 | VL | CDRL3 | S93L | 503 | 576 |
| AHF38366 | 0.636 | 0.645 | 0.986 | VL | CDRL3 | S93M | 504 | 576 |
| AHF38396 | 0.603 | 0.645 | 0.934 | VL | CDRL3 | S93N | 505 | 576 |
| AHF38367 | 0.579 | 0.645 | 0.897 | VL | CDRL3 | S93Q | 506 | 576 |
| AHF38402 | 0.696 | 0.645 | 1.078 | VL | CDRL3 | S93R | 507 | 576 |
| AHF38368 | 0.575 | 0.645 | 0.891 | VL | CDRL3 | S93T | 508 | 576 |
| AHF38377 | 0.593 | 0.645 | 0.919 | VL | CDRL3 | S93V | 509 | 576 |
| AHF38378 | 0.620 | 0.645 | 0.962 | VL | CDRL3 | S93W | 510 | 576 |
| AHF38436 | 0.386 | 0.707 | 0.546 | VL | CDRL3 | Y94A | 511 | 576 |
| AHF38413 | 0.103 | 0.707 | 0.146 | VL | CDRL3 | Y94D | 512 | 576 |
| AHF38448 | 0.294 | 0.707 | 0.416 | VL | CDRL3 | Y94E | 513 | 576 |
| AHF38425 | 0.058 | 0.707 | 0.082 | VL | CDRL3 | Y94F | 514 | 576 |
| AHF38454 | 0.288 | 0.707 | 0.407 | VL | CDRL3 | Y94G | 515 | 576 |
| AHF38444 | 0.424 | 0.707 | 0.600 | VL | CDRL3 | Y94I | 516 | 576 |
| AHF38410 | 0.136 | 0.707 | 0.192 | VL | CDRL3 | Y94K | 517 | 576 |
| AHF38445 | 0.431 | 0.707 | 0.609 | VL | CDRL3 | Y94L | 518 | 576 |
| AHF38439 | 0.370 | 0.707 | 0.524 | VL | CDRL3 | Y94N | 519 | 576 |
| AHF38421 | 0.282 | 0.707 | 0.399 | VL | CDRL3 | Y94P | 520 | 576 |
| AHF38432 | 0.312 | 0.707 | 0.442 | VL | CDRL3 | Y94Q | 521 | 576 |
| AHF38451 | 0.305 | 0.707 | 0.432 | VL | CDRL3 | Y94R | 522 | 576 |
| AHF38440 | 0.211 | 0.707 | 0.298 | VL | CDRL3 | Y94S | 523 | 576 |
| AHF38426 | 0.429 | 0.707 | 0.607 | VL | CDRL3 | Y94V | 524 | 576 |
| AHF38427 | 0.066 | 0.707 | 0.093 | VL | CDRL3 | Y94W | 525 | 576 |
| AHF38498 | 0.650 | 0.707 | 0.920 | VL | CDRL3 | P95A | 526 | 576 |
| AHF38483 | 0.520 | 0.707 | 0.736 | VL | CDRL3 | P95D | 527 | 576 |
| AHF38474 | 0.225 | 0.707 | 0.318 | VL | CDRL3 | P95E | 528 | 576 |
| AHF38470 | 0.090 | 0.707 | 0.128 | VL | CDRL3 | P95F | 529 | 576 |
| AHF38477 | 0.591 | 0.707 | 0.835 | VL | CDRL3 | P95G | 530 | 576 |
| AHF38478 | 0.542 | 0.707 | 0.766 | VL | CDRL3 | P95H | 531 | 576 |
| AHF38471 | 0.333 | 0.707 | 0.470 | VL | CDRL3 | P95K | 532 | 576 |
| AHF38463 | 0.252 | 0.707 | 0.357 | VL | CDRL3 | P95L | 533 | 576 |
| AHF38489 | 0.194 | 0.707 | 0.274 | VL | CDRL3 | P95M | 534 | 576 |
| AHF38488 | 0.720 | 0.707 | 1.018 | VL | CDRL3 | P95N | 535 | 576 |
| AHF38485 | 0.507 | 0.707 | 0.717 | VL | CDRL3 | P95P | 231 | 576 |
| AHF38480 | 0.165 | 0.707 | 0.234 | VL | CDRL3 | P95Q | 536 | 576 |
| AHF38484 | 0.134 | 0.707 | 0.190 | VL | CDRL3 | P95R | 537 | 576 |
| AHF38494 | 0.568 | 0.707 | 0.803 | VL | CDRL3 | P95S | 538 | 576 |
| AHF38479 | 0.256 | 0.707 | 0.362 | VL | CDRL3 | P95T | 539 | 576 |
| AHF38482 | 0.527 | 0.707 | 0.745 | VL | CDRL3 | P95V | 540 | 576 |
| AHF38490 | 0.087 | 0.707 | 0.124 | VL | CDRL3 | P95W | 541 | 576 |
| AHF38517 | 0.065 | 0.792 | 0.083 | VL | CDRL3 | L96A | 542 | 576 |
| AHF38510 | 0.052 | 0.792 | 0.065 | VL | CDRL3 | L96D | 543 | 576 |
| AHF38532 | 0.096 | 0.792 | 0.121 | VL | CDRL3 | L96E | 544 | 576 |
| AHF38507 | 0.077 | 0.792 | 0.097 | VL | CDRL3 | L96F | 545 | 576 |
| AHF38533 | 0.064 | 0.792 | 0.081 | VL | CDRL3 | L96G | 546 | 576 |
| AHF38541 | 0.059 | 0.792 | 0.074 | VL | CDRL3 | L96H | 547 | 576 |
| AHF38547 | 0.706 | 0.792 | 0.892 | VL | CDRL3 | L96I | 548 | 576 |
| AHF38513 | 0.341 | 0.792 | 0.431 | VL | CDRL3 | L96M | 549 | 576 |
| AHF38538 | 0.070 | 0.792 | 0.089 | VL | CDRL3 | L96N | 550 | 576 |
| AHF38545 | 0.050 | 0.792 | 0.064 | VL | CDRL3 | L96P | 551 | 576 |
| AHF38518 | 0.057 | 0.792 | 0.072 | VL | CDRL3 | L96Q | 552 | 576 |
| AHF38509 | 0.059 | 0.792 | 0.074 | VL | CDRL3 | L96R | 553 | 576 |
| AHF38512 | 0.058 | 0.792 | 0.074 | VL | CDRL3 | L96S | 554 | 576 |
| AHF38515 | 0.051 | 0.792 | 0.064 | VL | CDRL3 | L96T | 555 | 576 |
| AHF38548 | 0.511 | 0.792 | 0.646 | VL | CDRL3 | L96V | 556 | 576 |
| AHF38530 | 0.058 | 0.792 | 0.073 | VL | CDRL3 | L96W | 557 | 576 |
| AHF38528 | 0.090 | 0.792 | 0.114 | VL | CDRL3 | L96Y | 558 | 576 |
| AHF38591 | 0.813 | 0.792 | 1.026 | VL | CDRL3 | T97A | 559 | 576 |
| AHF38555 | 0.252 | 0.792 | 0.318 | VL | CDRL3 | T97D | 560 | 576 |
| AHF38582 | 0.617 | 0.792 | 0.779 | VL | CDRL3 | T97E | 561 | 576 |
| AHF38554 | 0.449 | 0.792 | 0.566 | VL | CDRL3 | T97F | 562 | 576 |
| AHF38584 | 0.836 | 0.792 | 1.055 | VL | CDRL3 | T97G | 563 | 576 |
| AHF38588 | 0.532 | 0.792 | 0.672 | VL | CDRL3 | T97H | 564 | 576 |
| AHF38589 | 0.556 | 0.792 | 0.702 | VL | CDRL3 | T97I | 565 | 576 |
| AHF38563 | 0.607 | 0.792 | 0.766 | VL | CDRL3 | T97L | 566 | 576 |
| AHF38552 | 0.510 | 0.792 | 0.644 | VL | CDRL3 | T97M | 567 | 576 |
| AHF38560 | 0.425 | 0.792 | 0.536 | VL | CDRL3 | T97N | 568 | 576 |
| AHF38576 | 0.059 | 0.792 | 0.074 | VL | CDRL3 | T97P | 569 | 576 |
| AHF38586 | 0.678 | 0.792 | 0.855 | VL | CDRL3 | T97Q | 570 | 576 |
| AHF38583 | 0.692 | 0.792 | 0.874 | VL | CDRL3 | T97R | 571 | 576 |
| AHF38572 | 0.763 | 0.792 | 0.963 | VL | CDRL3 | T97S | 572 | 576 |
| AHF38590 | 0.622 | 0.792 | 0.785 | VL | CDRL3 | T97T | 231 | 576 |
| AHF38581 | 0.677 | 0.792 | 0.854 | VL | CDRL3 | T97V | 573 | 576 |
| AHF38575 | 0.623 | 0.792 | 0.786 | VL | CDRL3 | T97W | 574 | 576 |

TABLE 14
Heavy Chain Mutant Fab Binding to Human TCRβV6-5 by ELISA

| | | | | Mutations | | | SEQ | |
|---|---|---|---|---|---|---|---|---|
| | Human TCRβV6-5-binding ELISA | | | in VH CDRs | | | ID NO | |
| | Mutation | WT | Ratio = OD | | | Point | Fab | Fab |
| ID | OD450nm | OD450nm | (Mutation)/OD(WT) | Chain | CDR | mutation | LC | HC |
| AHF36304 | 0.091 | 0.638 | 0.142 | VH | CDRH1 | Y27D | 231 | 594 |
| AHF36335 | 0.365 | 0.638 | 0.573 | VH | CDRH1 | Y27E | 231 | 595 |
| AHF36317 | 0.146 | 0.638 | 0.230 | VH | CDRH1 | Y27F | 231 | 596 |
| AHF36319 | 0.100 | 0.638 | 0.157 | VH | CDRH1 | Y27G | 231 | 597 |
| AHF36330 | 0.263 | 0.638 | 0.413 | VH | CDRH1 | Y27H | 231 | 598 |
| AHF36327 | 0.120 | 0.638 | 0.189 | VH | CDRH1 | Y27I | 231 | 599 |
| AHF36323 | 0.234 | 0.638 | 0.367 | VH | CDRH1 | Y27K | 231 | 600 |
| AHF36340 | 0.175 | 0.638 | 0.274 | VH | CDRH1 | Y27L | 231 | 601 |
| AHF36309 | 0.123 | 0.638 | 0.193 | VH | CDRH1 | Y27M | 231 | 602 |
| AHF36332 | 0.171 | 0.638 | 0.268 | VH | CDRH1 | Y27N | 231 | 603 |
| AHF36333 | 0.391 | 0.638 | 0.614 | VH | CDRH1 | Y27P | 231 | 604 |
| AHF36303 | 0.191 | 0.638 | 0.300 | VH | CDRH1 | Y27Q | 231 | 605 |
| AHF36293 | 0.109 | 0.638 | 0.170 | VH | CDRH1 | Y27R | 231 | 606 |
| AHF36331 | 0.214 | 0.638 | 0.335 | VH | CDRH1 | Y27S | 231 | 607 |
| AHF36339 | 0.187 | 0.638 | 0.293 | VH | CDRH1 | Y27T | 231 | 608 |
| AHF36336 | 0.235 | 0.638 | 0.368 | VH | CDRH1 | Y27V | 231 | 609 |
| AHF36295 | 0.094 | 0.638 | 0.147 | VH | CDRH1 | Y27W | 231 | 610 |
| AHF36379 | 0.709 | 0.638 | 1.112 | VH | CDRH1 | S28A | 231 | 611 |
| AHF36363 | 0.569 | 0.638 | 0.892 | VH | CDRH1 | S28E | 231 | 612 |
| AHF36368 | 0.383 | 0.638 | 0.600 | VH | CDRH1 | S28G | 231 | 613 |
| AHF36384 | 0.512 | 0.638 | 0.803 | VH | CDRH1 | S28H | 231 | 614 |
| AHF36366 | 0.563 | 0.638 | 0.883 | VH | CDRH1 | S28I | 231 | 615 |
| AHF36371 | 0.768 | 0.638 | 1.205 | VH | CDRH1 | S28K | 231 | 616 |
| AHF36345 | 0.595 | 0.638 | 0.933 | VH | CDRH1 | S28L | 231 | 617 |
| AHF36353 | 0.463 | 0.638 | 0.725 | VH | CDRH1 | S28M | 231 | 618 |
| AHF36359 | 0.624 | 0.638 | 0.979 | VH | CDRH1 | S28N | 231 | 619 |
| AHF36367 | 0.696 | 0.638 | 1.092 | VH | CDRH1 | S28P | 231 | 620 |
| AHF36380 | 0.617 | 0.638 | 0.968 | VH | CDRH1 | S28Q | 231 | 621 |
| AHF36351 | 0.659 | 0.638 | 1.034 | VH | CDRH1 | S28T | 231 | 622 |
| AHF36381 | 0.646 | 0.638 | 1.013 | VH | CDRH1 | S28V | 231 | 623 |
| AHF36374 | 0.299 | 0.638 | 0.468 | VH | CDRH1 | S28W | 231 | 624 |
| AHF36361 | 0.304 | 0.638 | 0.477 | VH | CDRH1 | S28Y | 231 | 625 |
| AHF36417 | 0.064 | 0.492 | 0.129 | VH | CDRH1 | F29A | 231 | 626 |
| AHF36419 | 0.063 | 0.492 | 0.129 | VH | CDRH1 | F29D | 231 | 627 |
| AHF36425 | 0.058 | 0.492 | 0.118 | VH | CDRH1 | F29E | 231 | 628 |
| AHF36403 | 0.049 | 0.492 | 0.100 | VH | CDRH1 | F29G | 231 | 629 |
| AHF36410 | 0.057 | 0.492 | 0.116 | VH | CDRH1 | F29H | 231 | 630 |
| AHF36409 | 0.286 | 0.492 | 0.582 | VH | CDRH1 | F29I | 231 | 631 |
| AHF36421 | 0.052 | 0.492 | 0.106 | VH | CDRH1 | F29K | 231 | 632 |
| AHF36432 | 0.171 | 0.492 | 0.348 | VH | CDRH1 | F29L | 231 | 633 |
| AHF36401 | 0.215 | 0.492 | 0.436 | VH | CDRH1 | F29M | 231 | 634 |
| AHF36422 | 0.067 | 0.492 | 0.136 | VH | CDRH1 | F29N | 231 | 635 |
| AHF36427 | 0.092 | 0.492 | 0.188 | VH | CDRH1 | F29P | 231 | 636 |
| AHF36408 | 0.172 | 0.492 | 0.351 | VH | CDRH1 | F29Q | 231 | 637 |
| AHF36397 | 0.048 | 0.492 | 0.098 | VH | CDRH1 | F29R | 231 | 638 |
| AHF36428 | 0.072 | 0.492 | 0.146 | VH | CDRH1 | F29S | 231 | 639 |
| AHF36424 | 0.060 | 0.492 | 0.121 | VH | CDRH1 | F29T | 231 | 640 |
| AHF36429 | 0.092 | 0.492 | 0.186 | VH | CDRHI | F29V | 231 | 641 |
| AHF36396 | 0.103 | 0.492 | 0.209 | VH | CDRH1 | F29W | 231 | 642 |
| AHF36391 | 0.067 | 0.492 | 0.137 | VH | CDRH1 | F29Y | 231 | 643 |
| AHF36463 | 0.323 | 0.492 | 0.657 | VH | CDRH1 | T30A | 231 | 644 |
| AHF36475 | 0.482 | 0.492 | 0.980 | VH | CDRH1 | T30E | 231 | 645 |
| AHF36439 | 0.429 | 0.492 | 0.874 | VH | CDRH1 | T30F | 231 | 646 |
| AHF36461 | 0.453 | 0.492 | 0.922 | VH | CDRH1 | T30G | 231 | 647 |
| AHF36437 | 0.341 | 0.492 | 0.693 | VH | CDRH1 | T30H | 231 | 648 |
| AHF36456 | 0.488 | 0.492 | 0.993 | VH | CDRH1 | T30I | 231 | 649 |
| AHF36458 | 0.417 | 0.492 | 0.848 | VH | CDRH1 | T30K | 231 | 650 |
| AHF36476 | 0.609 | 0.492 | 1.239 | VH | CDRH1 | T30L | 231 | 651 |
| AHF36457 | 0.401 | 0.492 | 0.816 | VH | CDRH1 | T30M | 231 | 652 |
| AHF36474 | 0.430 | 0.492 | 0.875 | VH | CDRH1 | T30N | 231 | 653 |
| AHF36466 | 0.175 | 0.492 | 0.356 | VH | CDRH1 | T30P | 231 | 654 |
| AHF36469 | 0.507 | 0.492 | 1.032 | VH | CDRH1 | T30Q | 231 | 655 |
| AHF36468 | 0.476 | 0.492 | 0.968 | VH | CDRH1 | T30R | 231 | 656 |
| AHF36446 | 0.396 | 0.492 | 0.805 | VH | CDRH1 | T30S | 231 | 657 |
| AHF36462 | 0.614 | 0.492 | 1.250 | VH | CDRH1 | T30V | 231 | 658 |
| AHF36470 | 0.517 | 0.492 | 1.052 | VH | CDRH1 | T30W | 231 | 659 |
| AHF36452 | 0.518 | 0.492 | 1.054 | VH | CDRH1 | T30Y | 231 | 660 |
| AHF36510 | 0.216 | 0.451 | 0.480 | VH | CDRH1 | T31A | 231 | 661 |
| AHF36479 | 0.196 | 0.451 | 0.435 | VH | CDRH1 | T31D | 231 | 662 |
| AHF36519 | 0.148 | 0.451 | 0.329 | VH | CDRH1 | T31E | 231 | 663 |
| AHF36494 | 0.240 | 0.451 | 0.532 | VH | CDRH1 | T31F | 231 | 664 |
| AHF36496 | 0.073 | 0.451 | 0.162 | VH | CDRH1 | T31G | 231 | 665 |
| AHF36516 | 0.373 | 0.451 | 0.826 | VH | CDRH1 | T31H | 231 | 666 |
| AHF36507 | 0.184 | 0.451 | 0.407 | VH | CDRH1 | T31I | 231 | 667 |
| AHF36508 | 0.329 | 0.451 | 0.730 | VH | CDRH1 | T31K | 231 | 668 |
| AHF36523 | 0.557 | 0.451 | 1.235 | VH | CDRH1 | T31L | 231 | 669 |
| AHF36522 | 0.043 | 0.451 | 0.095 | VH | CDRH1 | T31P | 231 | 670 |
| AHF36524 | 0.344 | 0.451 | 0.762 | VH | CDRH1 | T31Q | 231 | 671 |
| AHF36504 | 0.173 | 0.451 | 0.382 | VH | CDRH1 | T31R | 231 | 672 |
| AHF36486 | 0.230 | 0.451 | 0.510 | VH | CDRH1 | T31S | 231 | 673 |
| AHF36497 | 0.311 | 0.451 | 0.690 | VH | CDRH1 | T31T | 231 | 576 |
| AHF36495 | 0.192 | 0.451 | 0.427 | VH | CDRH1 | T31V | 231 | 674 |
| AHF36520 | 0.155 | 0.451 | 0.344 | VH | CDRH1 | T31W | 231 | 675 |
| AHF36517 | 0.372 | 0.451 | 0.824 | VH | CDRH1 | T31Y | 231 | 676 |
| AHF36544 | 0.273 | 0.451 | 0.606 | VH | CDRH1 | Y32A | 231 | 677 |
| AHF36548 | 0.064 | 0.451 | 0.141 | VH | CDRH1 | Y32D | 231 | 678 |
| AHF36557 | 0.164 | 0.451 | 0.364 | VH | CDRH1 | Y32E | 231 | 679 |
| AHF36560 | 0.553 | 0.451 | 1.226 | VH | CDRH1 | Y32F | 231 | 680 |
| AHF36561 | 0.370 | 0.451 | 0.820 | VH | CDRH1 | Y32G | 231 | 681 |
| AHF36564 | 0.071 | 0.451 | 0.157 | VH | CDRH1 | Y32H | 231 | 682 |
| AHF36541 | 0.347 | 0.451 | 0.769 | VH | CDRH1 | Y32I | 231 | 683 |
| AHF36556 | 0.054 | 0.451 | 0.120 | VH | CDRH1 | Y32K | 231 | 684 |
| AHF36551 | 0.511 | 0.451 | 1.132 | VH | CDRH1 | Y32L | 231 | 685 |
| AHF36552 | 0.529 | 0.451 | 1.173 | VH | CDRH1 | Y32M | 231 | 686 |
| AHF36545 | 0.061 | 0.451 | 0.134 | VH | CDRH1 | Y32N | 231 | 687 |
| AHF36558 | 0.087 | 0.451 | 0.193 | VH | CDRH1 | Y32P | 231 | 688 |
| AHF36531 | 0.149 | 0.451 | 0.331 | VH | CDRH1 | Y32Q | 231 | 689 |
| AHF36550 | 0.473 | 0.451 | 1.048 | VH | CDRH1 | Y32R | 231 | 690 |
| AHF36568 | 0.454 | 0.451 | 1.006 | VH | CDRH1 | Y32T | 231 | 691 |
| AHF36543 | 0.321 | 0.451 | 0.712 | VH | CDRH1 | Y32V | 231 | 692 |
| AHF36537 | 0.528 | 0.451 | 1.170 | VH | CDRH1 | Y32W | 231 | 693 |
| AHF36594 | 0.046 | 0.455 | 0.100 | VH | CDRH1 | H35A | 231 | 694 |
| AHF36576 | 0.066 | 0.455 | 0.145 | VH | CDRH1 | H35D | 231 | 695 |
| AHF36616 | 0.053 | 0.455 | 0.117 | VH | CDRH1 | H35E | 231 | 696 |
| AHF36582 | 0.057 | 0.455 | 0.126 | VH | CDRH1 | H35F | 231 | 697 |
| AHF36611 | 0.039 | 0.455 | 0.086 | VH | CDRH1 | H35G | 231 | 698 |
| AHF36579 | 0.046 | 0.455 | 0.101 | VH | CDRH1 | H35K | 231 | 699 |
| AHF36593 | 0.053 | 0.455 | 0.116 | VH | CDRH1 | H35L | 231 | 700 |
| AHF36591 | 0.050 | 0.455 | 0.111 | VH | CDRH1 | H35M | 231 | 701 |
| AHF36574 | 0.054 | 0.455 | 0.119 | VH | CDRH1 | H35N | 231 | 702 |
| AHF36584 | 0.046 | 0.455 | 0.102 | VH | CDRH1 | H35P | 231 | 703 |
| AHF36613 | 0.155 | 0.455 | 0.341 | VH | CDRH1 | H35Q | 231 | 704 |
| AHF36583 | 0.055 | 0.455 | 0.120 | VH | CDRH1 | H35S | 231 | 705 |
| AHF36600 | 0.053 | 0.455 | 0.116 | VH | CDRH1 | H35T | 231 | 706 |
| AHF36604 | 0.037 | 0.455 | 0.081 | VH | CDRH1 | H35V | 231 | 707 |
| AHF36608 | 0.054 | 0.455 | 0.118 | VH | CDRH1 | H35W | 231 | 708 |
| AHF36607 | 0.052 | 0.455 | 0.115 | VH | CDRH1 | H35Y | 231 | 709 |
| AHF36624 | 0.076 | 0.455 | 0.167 | VH | CDRH2 | W50A | 231 | 710 |
| AHF36625 | 0.196 | 0.455 | 0.431 | VH | CDRH2 | W50D | 231 | 711 |
| AHF36646 | 0.224 | 0.455 | 0.493 | VH | CDRH2 | W50E | 231 | 712 |
| AHF36631 | 0.039 | 0.455 | 0.086 | VH | CDRH2 | W50F | 231 | 713 |
| AHF36658 | 0.158 | 0.455 | 0.347 | VH | CDRH2 | W50G | 231 | 714 |
| AHF36657 | 0.168 | 0.455 | 0.370 | VH | CDRH2 | W50H | 231 | 715 |
| AHF36634 | 0.078 | 0.455 | 0.172 | VH | CDRH2 | W50I | 231 | 716 |
| AHF36645 | 0.076 | 0.455 | 0.168 | VH | CDRH2 | W50L | 231 | 717 |
| AHF36651 | 0.109 | 0.455 | 0.240 | VH | CDRH2 | W50M | 231 | 718 |
| AHF36633 | 0.093 | 0.455 | 0.204 | VH | CDRH2 | W50N | 231 | 719 |
| AHF36617 | 0.050 | 0.455 | 0.110 | VH | CDRH2 | W50P | 231 | 720 |
| AHF36642 | 0.123 | 0.455 | 0.270 | VH | CDRH2 | W50Q | 231 | 721 |
| AHF36643 | 0.190 | 0.455 | 0.418 | VH | CDRH2 | W50R | 231 | 722 |
| AHF36632 | 0.112 | 0.455 | 0.247 | VH | CDRH2 | W50S | 231 | 723 |
| AHF36660 | 0.069 | 0.455 | 0.152 | VH | CDRH2 | W50T | 231 | 724 |
| AHF36659 | 0.178 | 0.455 | 0.392 | VH | CDRH2 | W50V | 231 | 725 |
| AHF36647 | 0.081 | 0.455 | 0.178 | VH | CDRH2 | W50Y | 231 | 726 |
| AHF36681 | 0.277 | 0.612 | 0.453 | VH | CDRH2 | F51A | 231 | 727 |
| AHF36676 | 0.159 | 0.612 | 0.261 | VH | CDRH2 | F51D | 231 | 728 |
| AHF36694 | 0.471 | 0.612 | 0.770 | VH | CDRH2 | F51E | 231 | 729 |
| AHF36684 | 0.224 | 0.612 | 0.367 | VH | CDRH2 | F51G | 231 | 730 |
| AHF36704 | 0.452 | 0.612 | 0.740 | VH | CDRH2 | F51H | 231 | 731 |
| AHF36701 | 0.424 | 0.612 | 0.693 | VH | CDRH2 | F51K | 231 | 732 |
| AHF36706 | 0.384 | 0.612 | 0.628 | VH | CDRH2 | F51L | 231 | 733 |
| AHF36687 | 0.435 | 0.612 | 0.710 | VH | CDRH2 | F51M | 231 | 734 |
| AHF36698 | 0.371 | 0.612 | 0.607 | VH | CDRH2 | F51N | 231 | 735 |
| AHF36679 | 0.056 | 0.612 | 0.092 | VH | CDRH2 | F51P | 231 | 736 |
| AHF36693 | 0.419 | 0.612 | 0.684 | VH | CDRH2 | F51Q | 231 | 737 |
| AHF36667 | 0.228 | 0.612 | 0.372 | VH | CDRH2 | F51R | 231 | 738 |
| AHF36663 | 0.200 | 0.612 | 0.326 | VH | CDRH2 | F51S | 231 | 739 |
| AHF36677 | 0.295 | 0.612 | 0.481 | VH | CDRH2 | F51T | 231 | 740 |
| AHF36671 | 0.356 | 0.612 | 0.582 | VH | CDRH2 | F51V | 231 | 741 |
| AHF36697 | 0.070 | 0.612 | 0.114 | VH | CDRH2 | F51W | 231 | 742 |
| AHF36702 | 0.413 | 0.612 | 0.675 | VH | CDRH2 | F51Y | 231 | 743 |
| AHF36740 | 0.270 | 0.612 | 0.441 | VH | CDRH2 | F52A | 231 | 744 |
| AHF36717 | 0.084 | 0.612 | 0.137 | VH | CDRH2 | F52D | 231 | 745 |
| AHF36722 | 0.067 | 0.612 | 0.110 | VH | CDRH2 | F52E | 231 | 746 |
| AHF36752 | 0.100 | 0.612 | 0.163 | VH | CDRH2 | F52G | 231 | 747 |
| AHF36747 | 0.141 | 0.612 | 0.231 | VH | CDRH2 | F52H | 231 | 748 |
| AHF36738 | 0.060 | 0.612 | 0.097 | VH | CDRH2 | F52I | 231 | 749 |
| AHF36751 | 0.188 | 0.612 | 0.308 | VH | CDRH2 | F52K | 231 | 750 |
| AHF36716 | 0.070 | 0.612 | 0.115 | VH | CDRH2 | F52L | 231 | 751 |
| AHF36721 | 0.166 | 0.612 | 0.272 | VH | CDRH2 | F52M | 231 | 752 |
| AHF36750 | 0.176 | 0.612 | 0.288 | VH | CDRH2 | F52N | 231 | 753 |
| AHF36723 | 0.080 | 0.612 | 0.130 | VH | CDRH2 | F52P | 231 | 754 |
| AHF36725 | 0.089 | 0.612 | 0.145 | VH | CDRH2 | F52Q | 231 | 755 |
| AHF36742 | 0.358 | 0.612 | 0.585 | VH | CDRH2 | F52R | 231 | 756 |
| AHF36735 | 0.318 | 0.612 | 0.520 | VH | CDRH2 | F52S | 231 | 757 |
| AHF36712 | 0.066 | 0.612 | 0.108 | VH | CDRH2 | F52T | 231 | 758 |
| AHF36724 | 0.074 | 0.612 | 0.121 | VH | CDRH2 | F52V | 231 | 759 |
| AHF36749 | 0.186 | 0.612 | 0.304 | VH | CDRH2 | F52W | 231 | 760 |
| AHF36739 | 0.507 | 0.612 | 0.829 | VH | CDRH2 | F52Y | 231 | 761 |
| AHF36784 | 0.112 | 0.563 | 0.198 | VH | CDRH2 | P53A | 231 | 762 |
| AHF36754 | 0.053 | 0.563 | 0.094 | VH | CDRH2 | P53D | 231 | 763 |
| AHF36767 | 0.053 | 0.563 | 0.094 | VH | CDRH2 | P53E | 231 | 764 |
| AHF36790 | 0.054 | 0.563 | 0.096 | VH | CDRH2 | P53F | 231 | 765 |
| AHF36769 | 0.156 | 0.563 | 0.276 | VH | CDRH2 | P53G | 231 | 766 |
| AHF36791 | 0.090 | 0.563 | 0.161 | VH | CDRH2 | P53I | 231 | 767 |
| AHF36753 | 0.056 | 0.563 | 0.100 | VH | CDRH2 | P53K | 231 | 768 |
| AHF36770 | 0.066 | 0.563 | 0.117 | VH | CDRH2 | P53L | 231 | 769 |
| AHF36792 | 0.119 | 0.563 | 0.211 | VH | CDRH2 | P53N | 231 | 770 |
| AHF36776 | 0.057 | 0.563 | 0.102 | VH | CDRH2 | P53Q | 231 | 771 |
| AHF36793 | 0.054 | 0.563 | 0.096 | VH | CDRH2 | P53R | 231 | 772 |
| AHF36764 | 0.059 | 0.563 | 0.105 | VH | CDRH2 | P53S | 231 | 773 |
| AHF36765 | 0.066 | 0.563 | 0.117 | VH | CDRH2 | P53T | 231 | 774 |
| AHF36760 | 0.082 | 0.563 | 0.145 | VH | CDRH2 | P53V | 231 | 775 |
| AHF36777 | 0.048 | 0.563 | 0.085 | VH | CDRH2 | P53W | 231 | 776 |
| AHF36800 | 0.061 | 0.563 | 0.108 | VH | CDRH2 | P53Y | 231 | 777 |
| AHF36842 | 0.180 | 0.563 | 0.319 | VH | CDRH2 | G54D | 231 | 778 |
| AHF36808 | 0.424 | 0.563 | 0.753 | VH | CDRH2 | G54F | 231 | 779 |
| AHF36839 | 0.377 | 0.563 | 0.669 | VH | CDRH2 | G54H | 231 | 780 |
| AHF36818 | 0.360 | 0.563 | 0.640 | VH | CDRH2 | G54I | 231 | 781 |
| AHF36844 | 0.495 | 0.563 | 0.880 | VH | CDRH2 | G54K | 231 | 782 |
| AHF36830 | 0.441 | 0.563 | 0.784 | VH | CDRH2 | G54L | 231 | 783 |
| AHF36841 | 0.357 | 0.563 | 0.634 | VH | CDRH2 | G54M | 231 | 784 |
| AHF36831 | 0.167 | 0.563 | 0.297 | VH | CDRH2 | G54P | 231 | 785 |
| AHF36821 | 0.379 | 0.563 | 0.674 | VH | CDRH2 | G54Q | 231 | 786 |
| AHF36840 | 0.787 | 0.563 | 1.399 | VH | CDRH2 | G54R | 231 | 787 |
| AHF36838 | 0.334 | 0.563 | 0.594 | VH | CDRH2 | G54S | 231 | 788 |
| AHF36828 | 0.144 | 0.563 | 0.257 | VH | CDRH2 | G54T | 231 | 789 |
| AHF36834 | 0.108 | 0.563 | 0.191 | VH | CDRH2 | G54V | 231 | 790 |
| AHF36835 | 0.451 | 0.563 | 0.801 | VH | CDRH2 | G54W | 231 | 791 |
| AHF36803 | 0.403 | 0.563 | 0.716 | VH | CDRH2 | G54Y | 231 | 792 |
| AHF36879 | 0.091 | 0.600 | 0.152 | VH | CDRH2 | S55A | 231 | 793 |
| AHF36856 | 0.065 | 0.600 | 0.108 | VH | CDRH2 | S55D | 231 | 794 |
| AHF36870 | 0.064 | 0.600 | 0.106 | VH | CDRH2 | S55E | 231 | 795 |
| AHF36887 | 0.085 | 0.600 | 0.141 | VH | CDRH2 | S55F | 231 | 796 |
| AHF36875 | 0.206 | 0.600 | 0.344 | VH | CDRH2 | S55G | 231 | 797 |
| AHF36889 | 0.142 | 0.600 | 0.237 | VH | CDRH2 | S55H | 231 | 798 |
| AHF36872 | 0.081 | 0.600 | 0.135 | VH | CDRH2 | S55K | 231 | 799 |
| AHF36892 | 0.099 | 0.600 | 0.165 | VH | CDRH2 | S55L | 231 | 800 |
| AHF36848 | 0.075 | 0.600 | 0.124 | VH | CDRH2 | S55M | 231 | 801 |
| AHF36865 | 0.132 | 0.600 | 0.219 | VH | CDRH2 | S55N | 231 | 802 |
| AHF36861 | 0.058 | 0.600 | 0.097 | VH | CDRH2 | S55P | 231 | 803 |
| AHF36849 | 0.083 | 0.600 | 0.139 | VH | CDRH2 | S55Q | 231 | 804 |
| AHF36850 | 0.064 | 0.600 | 0.106 | VH | CDRH2 | S55R | 231 | 805 |
| AHF36891 | 0.245 | 0.600 | 0.409 | VH | CDRH2 | S55T | 231 | 806 |
| AHF36876 | 0.079 | 0.600 | 0.131 | VH | CDRH2 | S55V | 231 | 807 |
| AHF36883 | 0.177 | 0.600 | 0.296 | VH | CDRH2 | S55W | 231 | 808 |
| AHF36885 | 0.115 | 0.600 | 0.192 | VH | CDRH2 | S55Y | 231 | 809 |
| AHF36914 | 0.480 | 0.600 | 0.800 | VH | CDRH2 | G56A | 231 | 810 |
| AHF36915 | 0.347 | 0.600 | 0.579 | VH | CDRH2 | G56D | 231 | 811 |
| AHF36922 | 0.392 | 0.600 | 0.654 | VH | CDRH2 | G56E | 231 | 812 |
| AHF36931 | 0.476 | 0.600 | 0.793 | VH | CDRH2 | G56H | 231 | 813 |
| AHF36935 | 0.161 | 0.600 | 0.268 | VH | CDRH2 | G56I | 231 | 814 |
| AHF36894 | 0.280 | 0.600 | 0.466 | VH | CDRH2 | G56K | 231 | 815 |
| AHF36907 | 0.387 | 0.600 | 0.645 | VH | CDRH2 | G56L | 231 | 816 |
| AHF36923 | 0.448 | 0.600 | 0.746 | VH | CDRH2 | G56M | 231 | 817 |
| AHF36916 | 0.448 | 0.600 | 0.746 | VH | CDRH2 | G56N | 231 | 818 |
| AHF36904 | 0.091 | 0.600 | 0.152 | VH | CDRH2 | G56P | 231 | 819 |
| AHF36929 | 0.522 | 0.600 | 0.870 | VH | CDRH2 | G56Q | 231 | 820 |
| AHF36926 | 0.517 | 0.600 | 0.862 | VH | CDRH2 | G56R | 231 | 821 |
| AHF36920 | 0.497 | 0.600 | 0.828 | VH | CDRH2 | G56S | 231 | 822 |
| AHF36930 | 0.357 | 0.600 | 0.595 | VH | CDRH2 | G56T | 231 | 823 |
| AHF36928 | 0.151 | 0.600 | 0.251 | VH | CDRH2 | G56V | 231 | 824 |
| AHF36919 | 0.485 | 0.600 | 0.809 | VH | CDRH2 | G56W | 231 | 825 |
| AHF36913 | 0.418 | 0.600 | 0.697 | VH | CDRH2 | G56Y | 231 | 826 |
| AHF36939 | 0.075 | 0.622 | 0.121 | VH | CDRH2 | N57A | 231 | 827 |
| AHF36973 | 0.097 | 0.622 | 0.156 | VH | CDRH2 | N57D | 231 | 828 |
| AHF36975 | 0.068 | 0.622 | 0.109 | VH | CDRH2 | N57E | 231 | 829 |
| AHF36957 | 0.068 | 0.622 | 0.109 | VH | CDRH2 | N57F | 231 | 830 |
| AHF36948 | 0.172 | 0.622 | 0.276 | VH | CDRH2 | N57G | 231 | 831 |
| AHF36946 | 0.166 | 0.622 | 0.266 | VH | CDRH2 | N57H | 231 | 832 |
| AHF36955 | 0.098 | 0.622 | 0.158 | VH | CDRH2 | N57I | 231 | 833 |
| AHF36972 | 0.307 | 0.622 | 0.493 | VH | CDRH2 | N57K | 231 | 834 |
| AHF36978 | 0.113 | 0.622 | 0.181 | VH | CDRH2 | N57L | 231 | 835 |
| AHF36976 | 0.119 | 0.622 | 0.192 | VH | CDRH2 | N57M | 231 | 836 |
| AHF36945 | 0.056 | 0.622 | 0.090 | VH | CDRH2 | N57P | 231 | 837 |
| AHF36965 | 0.104 | 0.622 | 0.167 | VH | CDRH2 | N57Q | 231 | 838 |
| AHF36969 | 0.284 | 0.622 | 0.457 | VH | CDRH2 | N57R | 231 | 839 |
| AHF36947 | 0.198 | 0.622 | 0.318 | VH | CDRH2 | N57T | 231 | 840 |
| AHF36984 | 0.160 | 0.622 | 0.257 | VH | CDRH2 | N57V | 231 | 841 |
| AHF36967 | 0.058 | 0.622 | 0.093 | VH | CDRH2 | N57W | 231 | 842 |
| AHF36974 | 0.158 | 0.622 | 0.254 | VH | CDRH2 | N57Y | 231 | 843 |
| AHF37020 | 0.758 | 0.622 | 1.219 | VH | CDRH2 | I58A | 231 | 844 |
| AHF37022 | 0.507 | 0.622 | 0.816 | VH | CDRH2 | I58D | 231 | 845 |
| AHF37005 | 0.605 | 0.622 | 0.973 | VH | CDRH2 | I58E | 231 | 846 |
| AHF37000 | 0.530 | 0.622 | 0.853 | VH | CDRH2 | I58F | 231 | 847 |
| AHF37025 | 0.711 | 0.622 | 1.144 | VH | CDRH2 | I58K | 231 | 848 |
| AHF37021 | 0.759 | 0.622 | 1.222 | VH | CDRH2 | I58L | 231 | 849 |
| AHF37010 | 0.654 | 0.622 | 1.051 | VH | CDRH2 | I58M | 231 | 850 |
| AHF37006 | 0.547 | 0.622 | 0.879 | VH | CDRH2 | I58N | 231 | 851 |
| AHF37003 | 0.467 | 0.622 | 0.752 | VH | CDRH2 | I58P | 231 | 852 |
| AHF37019 | 0.717 | 0.622 | 1.154 | VH | CDRH2 | I58Q | 231 | 853 |
| AHF36990 | 0.541 | 0.622 | 0.870 | VH | CDRH2 | I58R | 231 | 854 |
| AHF36997 | 0.585 | 0.622 | 0.941 | VH | CDRH2 | I58S | 231 | 855 |
| AHF37017 | 0.656 | 0.622 | 1.056 | VH | CDRH2 | I58T | 231 | 856 |
| AHF36992 | 0.957 | 0.622 | 1.539 | VH | CDRH2 | I58V | 231 | 857 |
| AHF36986 | 0.366 | 0.622 | 0.589 | VH | CDRH2 | I58W | 231 | 858 |
| AHF37011 | 0.775 | 0.622 | 1.247 | VH | CDRH2 | I58Y | 231 | 859 |
| AHF37062 | 0.369 | 0.723 | 0.511 | VH | CDRH2 | K59A | 231 | 860 |
| AHF37054 | 0.065 | 0.723 | 0.090 | VH | CDRH2 | K59D | 231 | 861 |
| AHF37069 | 0.317 | 0.723 | 0.439 | VH | CDRH2 | K59E | 231 | 862 |
| AHF37070 | 0.428 | 0.723 | 0.593 | VH | CDRH2 | K59F | 231 | 863 |
| AHF37039 | 0.097 | 0.723 | 0.134 | VH | CDRH2 | K59G | 231 | 864 |
| AHF37059 | 0.477 | 0.723 | 0.660 | VH | CDRH2 | K59H | 231 | 865 |
| AHF37073 | 0.493 | 0.723 | 0.682 | VH | CDRH2 | K59L | 231 | 866 |
| AHF37071 | 0.544 | 0.723 | 0.753 | VH | CDRH2 | K59M | 231 | 867 |
| AHF37065 | 0.623 | 0.723 | 0.862 | VH | CDRH2 | K59N | 231 | 868 |
| AHF37066 | 0.072 | 0.723 | 0.099 | VH | CDRH2 | K59P | 231 | 869 |
| AHF37074 | 0.548 | 0.723 | 0.758 | VH | CDRH2 | K59Q | 231 | 870 |
| AHF37038 | 0.040 | 0.723 | 0.055 | VH | CDRH2 | K59R | 231 | 871 |
| AHF37061 | 0.418 | 0.723 | 0.579 | VH | CDRH2 | K59S | 231 | 872 |
| AHF37053 | 0.324 | 0.723 | 0.448 | VH | CDRH2 | K59T | 231 | 873 |
| AHF37072 | 0.349 | 0.723 | 0.483 | VH | CDRH2 | K59V | 231 | 874 |
| AHF37063 | 0.324 | 0.723 | 0.448 | VH | CDRH2 | K59W | 231 | 875 |
| AHF37114 | 0.729 | 0.723 | 1.008 | VH | CDRH2 | N61D | 231 | 876 |
| AHF37097 | 0.706 | 0.723 | 0.977 | VH | CDRH2 | N61E | 231 | 877 |
| AHF37107 | 0.546 | 0.723 | 0.756 | VH | CDRH2 | N61F | 231 | 878 |
| AHF37116 | 0.662 | 0.723 | 0.915 | VH | CDRH2 | N61G | 231 | 879 |
| AHF37089 | 0.766 | 0.723 | 1.059 | VH | CDRH2 | N61H | 231 | 880 |
| AHF37118 | 0.524 | 0.723 | 0.725 | VH | CDRH2 | N61I | 231 | 881 |
| AHF37110 | 0.536 | 0.723 | 0.741 | VH | CDRH2 | N61K | 231 | 882 |
| AHF37111 | 0.220 | 0.723 | 0.304 | VH | CDRH2 | N61L | 231 | 883 |
| AHF37104 | 0.639 | 0.723 | 0.884 | VH | CDRH2 | N61M | 231 | 884 |
| AHF37077 | 0.502 | 0.723 | 0.694 | VH | CDRH2 | N61N | 231 | 576 |
| AHF37115 | 0.760 | 0.723 | 1.051 | VH | CDRH2 | N61P | 231 | 885 |
| AHF37095 | 0.603 | 0.723 | 0.834 | VH | CDRH2 | N61Q | 231 | 886 |
| AHF37099 | 0.773 | 0.723 | 1.069 | VH | CDRH2 | N61S | 231 | 887 |
| AHF37091 | 0.751 | 0.723 | 1.039 | VH | CDRH2 | N61T | 231 | 888 |
| AHF37119 | 0.850 | 0.723 | 1.176 | VH | CDRH2 | N61V | 231 | 889 |
| AHF37103 | 0.673 | 0.723 | 0.930 | VH | CDRH2 | N61W | 231 | 890 |
| AHF37094 | 0.484 | 0.723 | 0.670 | VH | CDRH2 | N61Y | 231 | 891 |
| AHF33482 | 0.199 | 0.379 | 0.525 | VH | CDRH2 | E62A | 231 | 892 |
| AHF33488 | 0.191 | 0.379 | 0.503 | VH | CDRH2 | E62D | 231 | 893 |
| AHF33466 | 0.214 | 0.379 | 0.563 | VH | CDRH2 | E62F | 231 | 894 |
| AHF33472 | 0.150 | 0.379 | 0.397 | VH | CDRH2 | E62G | 231 | 895 |
| AHF33456 | 0.215 | 0.379 | 0.566 | VH | CDRH2 | E62H | 231 | 896 |
| AHF33487 | 0.224 | 0.379 | 0.590 | VH | CDRH2 | E62I | 231 | 897 |
| AHF33483 | 0.205 | 0.379 | 0.542 | VH | CDRH2 | E62K | 231 | 898 |
| AHF33477 | 0.192 | 0.379 | 0.506 | VH | CDRH2 | E62L | 231 | 899 |
| AHF33479 | 0.193 | 0.379 | 0.509 | VH | CDRH2 | E62M | 231 | 900 |
| AHF33494 | 0.221 | 0.379 | 0.584 | VH | CDRH2 | E62N | 231 | 901 |
| AHF33471 | 0.259 | 0.379 | 0.684 | VH | CDRH2 | E62P | 231 | 902 |
| AHF33491 | 0.228 | 0.379 | 0.601 | VH | CDRH2 | E62Q | 231 | 903 |
| AHF33480 | 0.189 | 0.379 | 0.498 | VH | CDRH2 | E62R | 231 | 904 |
| AHF33475 | 0.192 | 0.379 | 0.506 | VH | CDRH2 | E62S | 231 | 905 |
| AHF33452 | 0.151 | 0.379 | 0.399 | VH | CDRH2 | E62T | 231 | 906 |
| AHF33484 | 0.195 | 0.379 | 0.515 | VH | CDRH2 | E62V | 231 | 907 |
| AHF33467 | 0.215 | 0.379 | 0.568 | VH | CDRH2 | E62W | 231 | 908 |
| AHF33449 | 0.193 | 0.379 | 0.510 | VH | CDRH2 | E62Y | 231 | 909 |
| AHF33527 | 0.416 | 0.379 | 1.097 | VH | CDRH3 | G98A | 231 | 910 |
| AHF33536 | 0.056 | 0.379 | 0.147 | VH | CDRH3 | G98C | 231 | 911 |
| AHF33539 | 0.109 | 0.379 | 0.288 | VH | CDRH3 | G98D | 231 | 912 |
| AHF33520 | 0.073 | 0.379 | 0.192 | VH | CDRH3 | G98F | 231 | 913 |
| AHF33518 | 0.111 | 0.379 | 0.292 | VH | CDRH3 | G98I | 231 | 914 |
| AHF33504 | 0.071 | 0.379 | 0.186 | VH | CDRH3 | G98L | 231 | 915 |
| AHF33537 | 0.132 | 0.379 | 0.348 | VH | CDRH3 | G98M | 231 | 916 |
| AHF33538 | 0.088 | 0.379 | 0.233 | VH | CDRH3 | G98N | 231 | 917 |
| AHF33528 | 0.170 | 0.379 | 0.449 | VH | CDRH3 | G98P | 231 | 918 |
| AHF33534 | 0.091 | 0.379 | 0.241 | VH | CDRH3 | G98Q | 231 | 919 |
| AHF33506 | 0.048 | 0.379 | 0.127 | VH | CDRH3 | G98R | 231 | 920 |
| AHF33519 | 0.183 | 0.379 | 0.484 | VH | CDRH3 | G98V | 231 | 921 |
| AHF33517 | 0.056 | 0.379 | 0.148 | VH | CDRH3 | G98W | 231 | 922 |
| AHF37160 | 0.323 | 0.718 | 0.450 | VH | CDRH3 | S99A | 231 | 923 |
| AHF37133 | 0.053 | 0.718 | 0.074 | VH | CDRH3 | S99D | 231 | 924 |
| AHF37131 | 0.053 | 0.718 | 0.074 | VH | CDRH3 | S99E | 231 | 925 |
| AHF37166 | 0.040 | 0.718 | 0.056 | VH | CDRH3 | S99F | 231 | 926 |
| AHF37126 | 0.051 | 0.718 | 0.071 | VH | CDRH3 | S99G | 231 | 927 |
| AHF37150 | 0.052 | 0.718 | 0.072 | VH | CDRH3 | S99H | 231 | 928 |
| AHF37153 | 0.049 | 0.718 | 0.068 | VH | CDRH3 | S99I | 231 | 929 |
| AHF37145 | 0.056 | 0.718 | 0.078 | VH | CDRH3 | S99K | 231 | 930 |
| AHF37127 | 0.051 | 0.718 | 0.071 | VH | CDRH3 | S99L | 231 | 931 |
| AHF37167 | 0.044 | 0.718 | 0.062 | VH | CDRH3 | S99M | 231 | 932 |
| AHF37137 | 0.045 | 0.718 | 0.063 | VH | CDRH3 | S99N | 231 | 933 |
| AHF37152 | 0.168 | 0.718 | 0.234 | VH | CDRH3 | S99P | 231 | 934 |
| AHF37154 | 0.043 | 0.718 | 0.059 | VH | CDRH3 | S99R | 231 | 935 |
| AHF37158 | 0.050 | 0.718 | 0.070 | VH | CDRH3 | S99V | 231 | 936 |
| AHF37146 | 0.051 | 0.718 | 0.071 | VH | CDRH3 | S99W | 231 | 937 |
| AHF37135 | 0.051 | 0.718 | 0.070 | VH | CDRH3 | S99Y | 231 | 938 |
| AHF37183 | 0.056 | 0.718 | 0.078 | VH | CDRH3 | Y100A | 231 | 939 |
| AHF37204 | 0.059 | 0.718 | 0.082 | VH | CDRH3 | Y100D | 231 | 940 |
| AHF37181 | 0.053 | 0.718 | 0.074 | VH | CDRH3 | Y100E | 231 | 941 |
| AHF37192 | 0.216 | 0.718 | 0.301 | VH | CDRH3 | Y100F | 231 | 942 |
| AHF37171 | 0.060 | 0.718 | 0.083 | VH | CDRH3 | Y100H | 231 | 943 |
| AHF37178 | 0.141 | 0.718 | 0.197 | VH | CDRH3 | Y100I | 231 | 944 |
| AHF37198 | 0.052 | 0.718 | 0.072 | VH | CDRH3 | Y100K | 231 | 945 |
| AHF37202 | 0.140 | 0.718 | 0.194 | VH | CDRH3 | Y100L | 231 | 946 |
| AHF37170 | 0.076 | 0.718 | 0.106 | VH | CDRH3 | Y100M | 231 | 947 |
| AHF37173 | 0.050 | 0.718 | 0.070 | VH | CDRH3 | Y100N | 231 | 948 |
| AHF37199 | 0.052 | 0.718 | 0.072 | VH | CDRH3 | Y100P | 231 | 949 |
| AHF37206 | 0.058 | 0.718 | 0.080 | VH | CDRH3 | Y100Q | 231 | 950 |
| AHF37179 | 0.050 | 0.718 | 0.070 | VH | CDRH3 | Y100R | 231 | 951 |
| AHF37175 | 0.056 | 0.718 | 0.078 | VH | CDRH3 | Y100S | 231 | 952 |
| AHF37184 | 0.069 | 0.718 | 0.096 | VH | CDRH3 | Y100T | 231 | 953 |
| AHF37190 | 0.289 | 0.718 | 0.402 | VH | CDRH3 | Y100V | 231 | 954 |
| AHF37195 | 0.063 | 0.718 | 0.087 | VH | CDRH3 | Y100W | 231 | 955 |
| AHF37257 | 0.046 | 0.662 | 0.070 | VH | CDRH3 | Y101A | 231 | 956 |
| AHF37259 | 0.049 | 0.662 | 0.074 | VH | CDRH3 | Y101D | 231 | 957 |
| AHF37220 | 0.053 | 0.662 | 0.080 | VH | CDRH3 | Y101E | 231 | 958 |
| AHF37243 | 0.185 | 0.662 | 0.279 | VH | CDRH3 | Y101F | 231 | 959 |
| AHF37247 | 0.060 | 0.662 | 0.090 | VH | CDRH3 | Y101H | 231 | 960 |
| AHF37213 | 0.049 | 0.662 | 0.073 | VH | CDRH3 | Y101I | 231 | 961 |
| AHF37216 | 0.051 | 0.662 | 0.077 | VH | CDRH3 | Y101K | 231 | 962 |
| AHF37218 | 0.051 | 0.662 | 0.078 | VH | CDRH3 | Y101L | 231 | 963 |
| AHF37255 | 0.057 | 0.662 | 0.085 | VH | CDRH3 | Y101M | 231 | 964 |
| AHF37244 | 0.053 | 0.662 | 0.080 | VH | CDRH3 | Y101N | 231 | 965 |
| AHF37245 | 0.055 | 0.662 | 0.084 | VH | CDRH3 | Y101P | 231 | 966 |
| AHF37228 | 0.055 | 0.662 | 0.083 | VH | CDRH3 | Y101Q | 231 | 967 |
| AHF37225 | 0.055 | 0.662 | 0.082 | VH | CDRH3 | Y101S | 231 | 968 |
| AHF37234 | 0.054 | 0.662 | 0.081 | VH | CDRH3 | Y101T | 231 | 969 |
| AHF37260 | 0.068 | 0.662 | 0.102 | VH | CDRH3 | Y101V | 231 | 970 |
| AHF37256 | 0.425 | 0.662 | 0.642 | VH | CDRH3 | Y101W | 231 | 971 |
| AHF37266 | 0.312 | 0.662 | 0.470 | VH | CDRH3 | S102A | 231 | 972 |
| AHF37302 | 0.051 | 0.662 | 0.077 | VH | CDRH3 | S102D | 231 | 973 |
| AHF37296 | 0.048 | 0.662 | 0.072 | VH | CDRH3 | S102E | 231 | 974 |
| AHF37264 | 0.045 | 0.662 | 0.067 | VH | CDRH3 | S102F | 231 | 975 |
| AHF37288 | 0.157 | 0.662 | 0.237 | VH | CDRH3 | S102G | 231 | 976 |
| AHF37292 | 0.069 | 0.662 | 0.104 | VH | CDRH3 | S102H | 231 | 977 |
| AHF37267 | 0.058 | 0.662 | 0.087 | VH | CDRH3 | S102I | 231 | 978 |
| AHF37291 | 0.103 | 0.662 | 0.155 | VH | CDRH3 | S102L | 231 | 979 |
| AHF37287 | 0.103 | 0.662 | 0.155 | VH | CDRH3 | S102M | 231 | 980 |
| AHF37301 | 0.126 | 0.662 | 0.190 | VH | CDRH3 | S102N | 231 | 981 |
| AHF37276 | 0.068 | 0.662 | 0.102 | VH | CDRH3 | S102Q | 231 | 982 |
| AHF37289 | 0.048 | 0.662 | 0.073 | VH | CDRH3 | S102R | 231 | 983 |
| AHF37285 | 0.299 | 0.662 | 0.452 | VH | CDRH3 | S102T | 231 | 984 |
| AHF37268 | 0.071 | 0.662 | 0.106 | VH | CDRH3 | S102V | 231 | 985 |
| AHF37299 | 0.060 | 0.662 | 0.091 | VH | CDRH3 | S102W | 231 | 986 |
| AHF37269 | 0.070 | 0.662 | 0.106 | VH | CDRH3 | S102Y | 231 | 987 |
| AHF37316 | 0.058 | 0.654 | 0.088 | VH | CDRH3 | Y103A | 231 | 988 |
| AHF37341 | 0.059 | 0.654 | 0.090 | VH | CDRH3 | Y103D | 231 | 989 |
| AHF37307 | 0.053 | 0.654 | 0.080 | VH | CDRH3 | Y103E | 231 | 990 |
| AHF37318 | 0.636 | 0.654 | 0.973 | VH | CDRH3 | Y103F | 231 | 991 |
| AHF37324 | 0.063 | 0.654 | 0.097 | VH | CDRH3 | Y103H | 231 | 992 |
| AHF37343 | 0.052 | 0.654 | 0.079 | VH | CDRH3 | Y103I | 231 | 993 |
| AHF37325 | 0.088 | 0.654 | 0.135 | VH | CDRH3 | Y103K | 231 | 994 |
| AHF37333 | 0.064 | 0.654 | 0.097 | VH | CDRH3 | Y103L | 231 | 995 |
| AHF37323 | 0.129 | 0.654 | 0.197 | VH | CDRH3 | Y103M | 231 | 996 |
| AHF37308 | 0.050 | 0.654 | 0.077 | VH | CDRH3 | Y103N | 231 | 997 |
| AHF37342 | 0.143 | 0.654 | 0.218 | VH | CDRH3 | Y103P | 231 | 998 |
| AHF37322 | 0.051 | 0.654 | 0.078 | VH | CDRH3 | Y103Q | 231 | 999 |
| AHF37305 | 0.060 | 0.654 | 0.091 | VH | CDRH3 | Y103R | 231 | 1000 |
| AHF37321 | 0.065 | 0.654 | 0.100 | VH | CDRH3 | Y103S | 231 | 1001 |
| AHF37339 | 0.064 | 0.654 | 0.098 | VH | CDRH3 | Y103V | 231 | 1002 |
| AHF37334 | 0.067 | 0.654 | 0.102 | VH | CDRH3 | Y103W | 231 | 1003 |
| AHF37367 | 0.052 | 0.654 | 0.080 | VH | CDRH3 | D104E | 231 | 1004 |
| AHF37366 | 0.049 | 0.654 | 0.076 | VH | CDRH3 | D104F | 231 | 1005 |
| AHF37386 | 0.109 | 0.654 | 0.167 | VH | CDRH3 | D104G | 231 | 1006 |
| AHF37355 | 0.058 | 0.654 | 0.089 | VH | CDRH3 | D104H | 231 | 1007 |
| AHF37396 | 0.062 | 0.654 | 0.095 | VH | CDRH3 | D104K | 231 | 1008 |
| AHF37395 | 0.081 | 0.654 | 0.123 | VH | CDRH3 | D104L | 231 | 1009 |
| AHF37393 | 0.641 | 0.654 | 0.981 | VH | CDRH3 | D104N | 231 | 1010 |
| AHF37364 | 0.055 | 0.654 | 0.084 | VH | CDRH3 | D104P | 231 | 1011 |
| AHF37374 | 0.060 | 0.654 | 0.092 | VH | CDRH3 | D104Q | 231 | 1012 |
| AHF37394 | 0.054 | 0.654 | 0.083 | VH | CDRH3 | D104R | 231 | 1013 |
| AHF37380 | 0.194 | 0.654 | 0.297 | VH | CDRH3 | D104S | 231 | 1014 |
| AHF37376 | 0.065 | 0.654 | 0.099 | VH | CDRH3 | D104T | 231 | 1015 |
| AHF37361 | 0.046 | 0.654 | 0.070 | VH | CDRH3 | D104V | 231 | 1016 |
| AHF37362 | 0.051 | 0.654 | 0.077 | VH | CDRH3 | D104W | 231 | 1017 |
| AHF37443 | 0.149 | 0.665 | 0.223 | VH | CDRH3 | V105A | 231 | 1018 |
| AHF37419 | 0.063 | 0.665 | 0.095 | VH | CDRH3 | V105D | 231 | 1019 |
| AHF37403 | 0.078 | 0.665 | 0.117 | VH | CDRH3 | V105E | 231 | 1020 |
| AHF37444 | 0.063 | 0.665 | 0.094 | VH | CDRH3 | V105F | 231 | 1021 |
| AHF37413 | 0.059 | 0.665 | 0.089 | VH | CDRH3 | V105G | 231 | 1022 |
| AHF37428 | 0.066 | 0.665 | 0.099 | VH | CDRH3 | V105H | 231 | 1023 |
| AHF37420 | 0.066 | 0.665 | 0.100 | VH | CDRH3 | V105K | 231 | 1024 |
| AHF37426 | 0.056 | 0.665 | 0.084 | VH | CDRH3 | V105L | 231 | 1025 |
| AHF37397 | 0.067 | 0.665 | 0.101 | VH | CDRH3 | V105M | 231 | 1026 |
| AHF37421 | 0.123 | 0.665 | 0.185 | VH | CDRH3 | V105N | 231 | 1027 |
| AHF37402 | 0.068 | 0.665 | 0.102 | VH | CDRH3 | V105P | 231 | 1028 |
| AHF37411 | 0.094 | 0.665 | 0.141 | VH | CDRH3 | V105Q | 231 | 1029 |
| AHF37399 | 0.056 | 0.665 | 0.084 | VH | CDRH3 | V105R | 231 | 1030 |
| AHF37437 | 0.063 | 0.665 | 0.095 | VH | CDRH3 | V105S | 231 | 1031 |
| AHF37412 | 0.086 | 0.665 | 0.129 | VH | CDRH3 | V105T | 231 | 1032 |
| AHF37436 | 0.080 | 0.665 | 0.120 | VH | CDRH3 | V105W | 231 | 1033 |
| AHF37404 | 0.068 | 0.665 | 0.102 | VH | CDRH3 | V105Y | 231 | 1034 |
| AHF37485 | 0.077 | 0.665 | 0.116 | VH | CDRH3 | L106A | 231 | 1035 |
| AHF37483 | 0.050 | 0.665 | 0.075 | VH | CDRH3 | L106D | 231 | 1036 |
| AHF37484 | 0.058 | 0.665 | 0.088 | VH | CDRH3 | L106E | 231 | 1037 |
| AHF37478 | 0.062 | 0.665 | 0.093 | VH | CDRH3 | L106G | 231 | 1038 |
| AHF37447 | 0.065 | 0.665 | 0.098 | VH | CDRH3 | L106H | 231 | 1039 |
| AHF37480 | 0.511 | 0.665 | 0.769 | VH | CDRH3 | L106I | 231 | 1040 |
| AHF37471 | 0.061 | 0.665 | 0.092 | VH | CDRH3 | L106K | 231 | 1041 |
| AHF37455 | 0.269 | 0.665 | 0.404 | VH | CDRH3 | L106M | 231 | 1042 |
| AHF37481 | 0.061 | 0.665 | 0.092 | VH | CDRH3 | L106N | 231 | 1043 |
| AHF37486 | 0.074 | 0.665 | 0.111 | VH | CDRH3 | L106P | 231 | 1044 |
| AHF37468 | 0.066 | 0.665 | 0.100 | VH | CDRH3 | L106Q | 231 | 1045 |
| AHF37460 | 0.069 | 0.665 | 0.103 | VH | CDRH3 | L106R | 231 | 1046 |
| AHF37453 | 0.044 | 0.665 | 0.066 | VH | CDRH3 | L106S | 231 | 1047 |
| AHF37475 | 0.068 | 0.665 | 0.102 | VH | CDRH3 | L106T | 231 | 1048 |
| AHF37487 | 0.254 | 0.665 | 0.382 | VH | CDRH3 | L106V | 231 | 1049 |
| AHF37473 | 0.081 | 0.665 | 0.122 | VH | CDRH3 | L106Y | 231 | 1050 |
| AHF37509 | 0.054 | 0.678 | 0.079 | VH | CDRH3 | D107A | 231 | 1051 |
| AHF37510 | 0.060 | 0.678 | 0.088 | VH | CDRH3 | D107E | 231 | 1052 |
| AHF37493 | 0.048 | 0.678 | 0.071 | VH | CDRH3 | D107F | 231 | 1053 |
| AHF37500 | 0.055 | 0.678 | 0.081 | VH | CDRH3 | D107G | 231 | 1054 |
| AHF37498 | 0.060 | 0.678 | 0.089 | VH | CDRH3 | D107H | 231 | 1055 |
| AHF37504 | 0.062 | 0.678 | 0.092 | VH | CDRH3 | D107I | 231 | 1056 |
| AHF37523 | 0.046 | 0.678 | 0.068 | VH | CDRH3 | D107K | 231 | 1057 |
| AHF37532 | 0.041 | 0.678 | 0.060 | VH | CDRH3 | D107L | 231 | 1058 |
| AHF37489 | 0.057 | 0.678 | 0.084 | VH | CDRH3 | D107M | 231 | 1059 |
| AHF37536 | 0.055 | 0.678 | 0.081 | VH | CDRH3 | D107N | 231 | 1060 |
| AHF37508 | 0.048 | 0.678 | 0.071 | VH | CDRH3 | D107P | 231 | 1061 |
| AHF37491 | 0.064 | 0.678 | 0.095 | VH | CDRH3 | D107Q | 231 | 1062 |
| AHF37507 | 0.058 | 0.678 | 0.086 | VH | CDRH3 | D107R | 231 | 1063 |
| AHF37502 | 0.072 | 0.678 | 0.107 | VH | CDRH3 | D107S | 231 | 1064 |
| AHF37490 | 0.058 | 0.678 | 0.086 | VH | CDRH3 | D107T | 231 | 1065 |
| AHF37501 | 0.081 | 0.678 | 0.120 | VH | CDRH3 | D107V | 231 | 1066 |
| AHF37519 | 0.046 | 0.678 | 0.068 | VH | CDRH3 | D107W | 231 | 1067 |
| AHF37506 | 0.056 | 0.678 | 0.082 | VH | CDRH3 | D107Y | 231 | 1068 |
| AHF37551 | 0.103 | 0.678 | 0.152 | VH | CDRH3 | Y108A | 231 | 1069 |
| AHF37573 | 0.080 | 0.678 | 0.118 | VH | CDRH3 | Y108D | 231 | 1070 |
| AHF37543 | 0.121 | 0.678 | 0.179 | VH | CDRH3 | Y108E | 231 | 1071 |
| AHF37577 | 0.146 | 0.678 | 0.215 | VH | CDRH3 | Y108G | 231 | 1072 |
| AHF37549 | 0.139 | 0.678 | 0.204 | VH | CDRH3 | Y108H | 231 | 1073 |
| AHF37557 | 0.092 | 0.678 | 0.135 | VH | CDRH3 | Y108I | 231 | 1074 |
| AHF37545 | 0.064 | 0.678 | 0.095 | VH | CDRH3 | Y108K | 231 | 1075 |
| AHF37570 | 0.070 | 0.678 | 0.103 | VH | CDRH3 | Y108L | 231 | 1076 |
| AHF37552 | 0.112 | 0.678 | 0.166 | VH | CDRH3 | Y108M | 231 | 1077 |
| AHF37548 | 0.082 | 0.678 | 0.122 | VH | CDRH3 | Y108N | 231 | 1078 |
| AHF37568 | 0.059 | 0.678 | 0.087 | VH | CDRH3 | Y108P | 231 | 1079 |
| AHF37558 | 0.095 | 0.678 | 0.141 | VH | CDRH3 | Y108Q | 231 | 1080 |
| AHF37580 | 0.057 | 0.678 | 0.085 | VH | CDRH3 | Y108R | 231 | 1081 |
| AHF37561 | 0.124 | 0.678 | 0.184 | VH | CDRH3 | Y108T | 231 | 1082 |
| AHF37546 | 0.119 | 0.678 | 0.175 | VH | CDRH3 | Y108V | 231 | 1083 |
| AHF37571 | 0.290 | 0.678 | 0.428 | VH | CDRH3 | Y108W | 231 | 1084 |

TABLE 15
Mutant Fab Binding to Human TCRβV6-5 by Biacore (SPR)

| | | | | | | | | Ratio = | Ratio = |
|---|---|---|---|---|---|---|---|---|---|
| | | | | | | | | WT(kd)/ | WT(KD)/ |
| | | | Mutation | | | | Rmax | mutant | mutant |
| ID | Fab ID | Mutation | location | ka (1/Ms) | kd (1/s) | KD (M) | (RU) | (kd) | (KD) |
| | WT | none | | 2.76E+04 | 4.98E−04 | 1.84E−08 | 28.3 | — | — |
| AHF36371 | AHF31254 | S28K | CDRH1 | 2.43E+04 | 3.37E−04 | 1.39E−08 | 32.5 | 1.48 | 1.32 |
| AHF36379 | AHF31255 | S28A | CDRH1 | 2.89E+04 | 4.20E−04 | 1.45E−08 | 56.4 | 1.19 | 1.27 |
| AHF36452 | AHF31257 | T30Y | CDRH1 | 2.58E+04 | 5.43E−04 | 2.10E−08 | 27.6 | 0.92 | 0.87 |
| AHF36462 | AHF31259 | T30V | CDRHI | 2.84E+04 | 4.09E−04 | 1.44E−08 | 30.3 | 1.22 | 1.27 |
| AHF36476 | AHF31260 | T30L | CDRH1 | 2.93E+04 | 4.44E−04 | 1.52E−08 | 27.8 | 1.12 | 1.21 |
| AHF36523 | AHF31261 | T31L | CDRH1 | 2.39E+04 | 2.66E−04 | 1.11E−08 | 16.9 | 1.87 | 1.65 |
| AHF36537 | AHF31262 | Y32W | CDRH1 | 2.25E+04 | 3.27E−04 | 1.46E−08 | 29.1 | 1.52 | 1.26 |
| AHF36551 | AHF31263 | Y32L | CDRHI | 3.74E+04 | 5.95E−04 | 1.59E−08 | 30.3 | 0.84 | 1.15 |
| AHF36552 | AHF31264 | Y32M | CDRH1 | 3.87E+04 | 5.20E−04 | 1.34E−08 | 30.2 | 0.96 | 1.37 |
| AHF36560 | AHF31265 | Y32F | CDRH1 | 2.80E+04 | 4.05E−04 | 1.45E−08 | 22.6 | 1.23 | 1.27 |
| AHF36840 | AHF31269 | G54R | CDRH2 | 2.75E+04 | 2.61E−04 | 9.50E−09 | 30.3 | 1.91 | 1.93 |
| AHF36992 | AHF31270 | I58V | CDRH2 | 2.80E+04 | 3.51E−04 | 1.25E−08 | 49.1 | 1.42 | 1.47 |
| AHF37011 | AHF31271 | I58Y | CDRH2 | 3.02E+04 | 4.15E−04 | 1.37E−08 | 39.9 | 1.20 | 1.34 |
| AHF37019 | AHF31272 | I58Q | CDRH2 | 2.89E+04 | 3.81E−04 | 1.32E−08 | 25.2 | 1.31 | 1.39 |
| AHF37020 | AHF31273 | I58A | CDRH2 | 2.76E+04 | 4.63E−04 | 1.68E−08 | 46.2 | 1.08 | 1.09 |
| AHF37021 | AHF31274 | I58L | CDRH2 | 3.03E+04 | 3.89E−04 | 1.28E−08 | 45.3 | 1.28 | 1.43 |
| AHF37089 | AHF31275 | N61H | CDRH2 | 2.60E+04 | 4.49E−04 | 1.73E−08 | 34.7 | 1.11 | 1.06 |
| AHF37099 | AHF31276 | N61S | CDRH2 | 2.80E+04 | 4.05E−04 | 1.45E−08 | 22.2 | 1.23 | 1.27 |
| AHF37115 | AHF31278 | N61P | CDRH2 | 3.05E+04 | 3.66E−04 | 1.20E−08 | 29.2 | 1.36 | 1.53 |
| AHF37119 | AHF31279 | N61V | CDRH2 | 2.58E+04 | 4.76E−04 | 1.84E−08 | 25.8 | 1.05 | 1.00 |
| AHF33527 | AHF31280 | G98A | CDRH3 | 3.27E+04 | 2.63E−04 | 8.05E−09 | 25.9 | 1.89 | 2.28 |
| AHF37318 | AHF31281 | Y103F | CDRH3 | 3.69E+04 | 3.41E−04 | 9.25E−09 | 37.5 | 1.46 | 1.98 |
| AHF37393 | AHF31282 | D104N | CDRH3 | 3.51E+04 | 4.36E−04 | 1.24E−08 | 27.6 | 1.14 | 1.48 |
| AHF37641 | AHF31283 | V29A | CDRL1 | 2.80E+04 | 4.12E−04 | 1.47E−08 | 29.7 | 1.21 | 1.25 |
| AHF37643 | AHF31284 | V29L | CDRL1 | 3.15E+04 | 4.20E−04 | 1.33E−08 | 41.6 | 1.19 | 1.38 |
| AHF37658 | AHF31286 | V29M | CDRL1 | 2.95E+04 | 4.86E−04 | 1.65E−08 | 43.3 | 1.02 | 1.11 |
| AHF37672 | AHF31287 | V29G | CDRL1 | 2.83E+04 | 4.57E−04 | 1.61E−08 | 21.2 | 1.09 | 1.14 |
| AHF37847 | AHF31289 | V33D | CDRLI | 2.93E+04 | 4.03E−04 | 1.38E−08 | 15.1 | 1.24 | 1.33 |
| AHF38156 | AHF31291 | S56K | CDRL2 | 2.47E+04 | 1.57E−04 | 6.33E−09 | 32.4 | 3.17 | 2.90 |
| AHF38172 | AHF31292 | S56R | CDRL2 | 2.02E+04 | 2.37E−04 | 1.18E−08 | 34.2 | 2.10 | 1.56 |
| AHF38402 | AHF31294 | S93R | CDRL3 | 2.57E+04 | 3.87E−04 | 1.51E−08 | 40.6 | 1.29 | 1.22 |
| AHF38408 | AHF31295 | S93F | CDRL3 | 2.58E+04 | 3.72E−04 | 1.44E−08 | 24.9 | 1.34 | 1.27 |
| AHF38488 | AHF31296 | P95N | CDRL3 | 3.00E+04 | 4.27E−04 | 1.42E−08 | 33.5 | 1.17 | 1.29 |
| AHF38547 | AHF31297 | L96I | CDRL3 | 3.13E+04 | 3.22E−04 | 1.03E−08 | 63.3 | 1.55 | 1.78 |
| AHF38584 | AHF31298 | T97G | CDRL3 | 2.91E+04 | 3.91E−04 | 1.35E−08 | 52.5 | 1.27 | 1.36 |
| AHF38591 | AHF31299 | T97A | CDRL3 | 2.60E+04 | 4.18E−04 | 1.61E−08 | 54.3 | 1.19 | 1.14 |
| | Blank | | | NA | NA | NA | NA | | |
| | Negative | | | NA | NA | NA | NA | | |
| | control | | | | | | | | |

TABLE 16
Mutant Fab Binding to Human TCRβV6-5 by Biacore (SPR)

| | | | | | | | | Ratio = | Ratio = |
|---|---|---|---|---|---|---|---|---|---|
| | | | | | | | | WT(kd)/ | WT(KD)/ |
| | | | Mutation | | | | Rmax | mutant | mutant |
| ID | Fab ID | Mutation | location | ka (1/Ms) | kd (1/s) | KD (M) | (RU) | (kd) | (KD) |
| | WT | none | | 2.57E+04 | 5.67E−04 | 2.21E−08 | 61.4 | — | — |
| AHF37619 | AHF32383 | N28P | CDRL1 | 2.65E+04 | 5.25E−04 | 1.98E−08 | 95.5 | 1.08 | 1.12 |
| AHF37604 | AHF32384 | N28D | CDRL1 | 2.57E+04 | 5.43E−04 | 2.12E−08 | 143.7 | 1.04 | 1.04 |
| AHF37587 | AHF32385 | N28Q | CDRL1 | 2.32E+04 | 5.70E−04 | 2.45E−08 | 80.8 | 0.99 | 0.90 |
| AHF37607 | AHF32386 | N28F | CDRL1 | 2.62E+04 | 5.53E−04 | 2.11E−08 | 72.8 | 1.03 | 1.05 |
| AHF37591 | AHF32387 | N28Y | CDRL1 | 2.47E+04 | 5.73E−04 | 2.32E−08 | 74.8 | 0.99 | 0.95 |
| Fab HC SEQ | AHF32388 | V29R | CDRL1 | 2.27E+04 | 5.96E−04 | 2.62E−08 | 49 | 0.95 | 0.84 |
| ID NO: 576 | | | | | | | | | |
| Fab LC SEQ | | | | | | | | | |
| ID NO: 1095 | | | | | | | | | |
| AHF37635 | AHF32391 | V29P | CDRL1 | 2.67E+04 | 5.51E−04 | 2.06E−08 | 69.5 | 1.03 | 1.07 |
| AHF37718 | AHF32393 | G30W | CDRL1 | 2.30E+04 | 5.55E−04 | 2.41E−08 | 47.6 | 1.02 | 0.92 |
| Fab HC SEQ | AHF32395 | N32R | CDRL1 | 1.90E+04 | 4.63E−04 | 2.44E−08 | 40.1 | 1.22 | 0.91 |
| ID NO: 576 | | | | | | | | | |
| Fab LC SEQ | | | | | | | | | |
| ID NO: 1096 | | | | | | | | | |
| AHF37994 | AHF32396 | S52W | CDRL2 | 2.30E+04 | 5.67E−04 | 2.47E−08 | 59.5 | 1.00 | 0.89 |
| AHF38044 | AHF32397 | R54I | CDRL2 | 2.06E+04 | 4.99E−04 | 2.42E−08 | 64.5 | 1.14 | 0.91 |
| AHF38084 | AHF32398 | R54V | CDRL2 | 1.95E+04 | 5.79E−04 | 2.97E−08 | 86.8 | 0.98 | 0.74 |
| Fab HC SEQ | AHF32402 | S56M | CDRL2 | 1.76E+04 | 4.39E−04 | 2.50E−08 | 60.5 | 1.29 | 0.88 |
| ID NO: 576 | | | | | | | | | |
| Fab LC SEQ | | | | | | | | | |
| ID NO: 1097 | | | | | | | | | |
| AHF38248 | AHF32407 | Q90E | CDRL3 | 2.14E+04 | 6.22E−04 | 2.91E−08 | 100 | 0.91 | 0.76 |
| Fab HC SEQ | AHF32408 | Q90S | CDRL3 | 1.91E+04 | 5.77E−04 | 3.02E−08 | 35 | 0.98 | 0.73 |
| ID NO: 576 | | | | | | | | | |
| Fab HC SEQ | | | | | | | | | |
| ID NO: 1098 | | | | | | | | | |
| AHF38538 | AHF32410 | L96N | CDRL3 | 2.66E+04 | 4.17E−04 | 1.57E−08 | 76.8 | 1.36 | 1.41 |
| AHF38572 | AHF32413 | T97S | CDRL3 | 2.52E+04 | 4.74E−04 | 1.88E−08 | 86.7 | 1.20 | 1.18 |
| | Blank | | | NA | NA | NA | NA | | |
| | Negative | | | NA | NA | NA | NA | | |
| | control | | | | | | | | |

TABLE 17
Combinatorial Mutant Fab Binding to Human TCRβV6-5 by ELISA

| | | | Human TCRβV6-5-binding ELISA | | | | |
|---|---|---|---|---|---|---|---|
| | | | Mutation | WT | Ratio = | SEQ ID NO | |
| | Mutations | | OD | OD | OD(Mutation)/ | Fab | Fab |
| ID | VH | VL | 450 nm | 450 nm | OD(WT) | HC | LC |
| WT | — | — | — | 0.34 | — | 576 | 231 |
| AHF40553 | G54R, G98A, Y103F | N32R, S56R, L96I | 0.97 | 0.34 | 2.84 | 583 | 238 |
| AHF40555 | T31L, G54R, G98A, Y103F | N32R, S93F, L96I | 0.97 | 0.34 | 2.86 | 587 | 237 |
| AHF40556 | Y32W, G98A, Y103F | N32R, S56K, S93F, L96I | 0.97 | 0.34 | 2.87 | 1099 | 243 |
| AHF40557 | T31L, Y32W, G98A, Y103F | N32R, L96I | 0.94 | 0.34 | 2.78 | 585 | 236 |
| AHF40559 | T31L, Y32W, G98A, Y103F | S56R, S93F, L96I | 1.01 | 0.34 | 2.98 | 583 | 237 |
| AHF40560 | T31L, G54R, G98A, Y103F | N32R, L96I | 1.01 | 0.34 | 2.97 | 585 | 242 |
| AHF40561 | G54R, G98A, Y103F | N32R, S56K, S93F, L96I | 0.99 | 0.34 | 2.93 | 587 | 236 |
| AHF40562 | T31L, Y32W, G54R, G98A, | S56K, L96I | 1.02 | 0.34 | 3.01 | 583 | 243 |
| | Y103F | | | | | | |
| AHF40563 | T31L, G98A, Y103F | N32R, S56K, S93F, L96I | 0.80 | 0.34 | 2.34 | 586 | 239 |
| AHF40564 | T31L, Y32W, G54R, G98A, | N32R, S56R, L96I | 0.79 | 0.34 | 2.33 | 582 | 243 |
| | Y103F | | | | | | |
| AHF40565 | T31L, G98A, Y103F | N32R, L96I | 0.94 | 0.34 | 2.77 | 586 | 238 |
| AHF40566 | G54R, G98A, Y103F | N32R, S93F, L96I | 0.94 | 0.34 | 2.76 | 582 | 236 |
| AHF40567 | T31L, G54R, G98A, Y103F | N32R, S56K, L96I | 0.92 | 0.34 | 2.70 | 587 | 240 |
| AHF40568 | T31L, G54R, G98A, Y103F | S56R, L96I | 0.81 | 0.34 | 2.40 | 587 | 241 |
| AHF40569 | T31L, Y32W, G54R, G98A, | N32R, S56R, S93F, L96I | 0.85 | 0.34 | 2.50 | 586 | 244 |
| | Y103F | | | | | | |
| AHF40570 | G54R, G98A, Y103F | S56R, L96I | 0.92 | 0.34 | 2.71 | 583 | 241 |
| AHF40571 | G98A, Y103F | N32R, S56R, L96I | 0.94 | 0.34 | 2.76 | 584 | 238 |
| AHF40572 | G54R, G98A, Y103F | N32R, L96I | 0.85 | 0.34 | 2.49 | 583 | 236 |

TABLE 18
Combinatorial Mutant Fab Binding to Human TCRβV6-5 by Biacore

| | | | Human TCRβV6-5-binding Biacore (SPR) | | | | | |
|---|---|---|---|---|---|---|---|---|
| | | | | | | | Ratio = | Ratio = |
| | | | | | | | WT(kd)/ | WT(KD)/ |
| | Mutations | | | | | Rmax | mutant | mutant |
| ID | VH | VL | ka (1/Ms) | kd (1/s) | KD (M) | (RU) | (kd) | (KD) |
| WT | — | — | 2.13E+04 | 6.10E−04 | 2.86E−08 | 65.1 | | |
| AHF40553 | G54R, G98A, Y103F | N32R, S56R, L96I | 1.55E+04 | 5.36E−04 | 3.45E−08 | 46.6 | 1.14 | 0.83 |
| AHF40555 | T31L, G54R, G98A, Y103F | N32R, S93F, L96I | 1.13E+04 | 4.44E−04 | 3.94E−08 | 49.5 | 1.37 | 0.73 |
| AHF40556 | Y32W, G98A, Y103F | N32R, S56K, S93F, | 1.63E+04 | 4.74E−04 | 2.91E−08 | 39.2 | 1.29 | 0.98 |
| | | L96I | | | | | | |
| AHF40557 | T31L, Y32W, G98A, Y103F | N32R, L96I | 1.62E+04 | 4.17E−04 | 2.57E−08 | 32.5 | 1.46 | 1.11 |
| AHF40559 | T31L, Y32W, G98A, Y103F | S56R, S93F, L96I | 5.05E+03 | 5.10E−04 | 1.01E−07 | 40.2 | 1.20 | 0.28 |
| AHF40560 | T31L, G54R, G98A, Y103F | N32R, L96I | 1.43E+04 | 9.77E−04 | 6.83E−08 | 23.2 | 0.62 | 0.42 |
| AHF40561 | G54R, G98A, Y103F | N32R, S56K, S93F, | 1.57E+04 | 4.71E−04 | 3.00E−08 | 31.4 | 1.30 | 0.95 |
| | | L96I | | | | | | |
| AHF40562 | T31L, Y32W, G54R, G98A, | S56K, L96I | 1.15E+04 | 3.89E−04 | 3.39E−08 | 39.7 | 1.57 | 0.84 |
| | Y103F | | | | | | | |
| AHF40563 | T21L, G98A, Y103F | N32R, S56K, S93F, | 1.34E+04 | 9.54E−04 | 7.11E−08 | 21.5 | 0.64 | 0.40 |
| | | L96I | | | | | | |
| AHF40564 | T31L, Y32W, G54R, G98A, | N32R, S56R, L96I | 1.50E+04 | 1.31E−03 | 8.71E−08 | 17.5 | 0.47 | 0.33 |
| | Y103F | | | | | | | |
| AHF40565 | T21L, G98A, Y103F | N32R, L96I | 1.21E+04 | 3.38E−04 | 2.79E−08 | 39.1 | 1.80 | 1.03 |
| AHF40566 | G54R, G98A, Y103F | N32R, S93F, L96I | 1.92E+04 | 3.19E−04 | 1.66E−08 | 45.4 | 1.91 | 1.72 |
| AHF40567 | T31L, G54R, G98A, Y103F | N32R, S56K, L96I | 1.78E+04 | 4.09E−04 | 2.29E−08 | 33.4 | 1.49 | 1.25 |
| AHF40568 | T31L, G54R, G98A, Y103F | S56R, L96I | 8.73E+03 | 5.62E−04 | 6.43E−08 | 33.3 | 1.09 | 0.44 |
| AHF40569 | T31L, Y32W, G54R, G98A, | N32R, S56R, S93F, | 1.15E+04 | 1.82E−03 | 1.58E−07 | 17.0 | 0.34 | 0.18 |
| | Y103F | L96I | | | | | | |
| AHF40570 | G54R, G98A, Y103F | S56R, L96I | 2.09E+04 | 3.53E−04 | 1.68E−08 | 53.4 | 1.73 | 1.70 |
| AHF40571 | G98A, Y103F | N32R, S56R, L96I | 1.33E+04 | 2.70E−04 | 2.03E−08 | 49.7 | 2.26 | 1.41 |
| AHF40572 | G54R, G98A, Y103F | N32R, L96I | 2.11E+04 | 3.13E−04 | 1.49E−08 | 53.3 | 1.95 | 1.92 |
| Negative | — | — | NA | NA | NA | NA | — | — |
| control | | | | | | | | |
| Blank | — | — | NA | NA | NA | NA | — | — |

TABLE 19
Combinatorial Mutant Fab Binding to Cyno PBMCs by Flow Cytometry

| | | | Cyno PBMC Binding by Flow Cytometry | | | |
|---|---|---|---|---|---|---|
| | | | | | % positive | Ratio = |
| | Mutations | | Mutation | WT | of cyano | GMFI(Mutation)/ |
| ID | VH | VL | GMFI | GMFI | T cells | GMFI(WT) |
| WT | — | — | | 160 | 4.6 | |
| AHF40553 | G54R, G98A, Y103F | N32R, S56R, L96I | 261 | 160 | 18.4 | 1.63 |
| AHF40555 | T31L, G54R, G98A, Y103F | N32R, S93F, L96I | 235 | 160 | 17 | 1.47 |
| AHF40556 | Y32W, G98A, Y103F | N32R, S56K, S93F, | 294 | 160 | 20.5 | 1.84 |
| | | L96I | | | | |
| AHF40557 | T31L, Y32W, G98A, Y103F | N32R, L96I | 261 | 160 | 18.7 | 1.63 |
| AHF40559 | T31L, Y32W, G98A, Y103F | S56R, S93F, L96I | 156 | 160 | 2.66 | 0.98 |
| AHF40560 | T31L, G54R, G98A, Y103F | N32R, L96I | 201 | 160 | 16.3 | 1.26 |
| AHF40561 | G54R, G98A, Y103F | N32R, S56K, S93F, | 215 | 160 | 14.8 | 1.34 |
| | | L96I | | | | |
| AHF40562 | T31L, Y32W, G54R, G98A, | S56K, L96I | 266 | 160 | 18.2 | 1.66 |
| | Y103F | | | | | |
| AHF40563 | T21L, G98A, Y103F | N32R, S56K, S93F, | 247 | 160 | 16.1 | 1.54 |
| | | L96I | | | | |
| AHF40564 | T31L, Y32W, G54R, G98A, | N32R, S56R, L96I | 265 | 160 | 16 | 1.66 |
| | Y103F | | | | | |
| AHF40565 | T21L, G98A, Y103F | N32R, L96I | 262 | 160 | 19 | 1.64 |
| AHF40566 | G54R, G98A, Y103F | N32R, S93F, L96I | 191 | 160 | 14.6 | 1.19 |
| AHF40567 | T31L, G54R, G98A, Y103F | N32R, S56K, L96I | 302 | 160 | 22.7 | 1.89 |
| AHF40568 | T31L, G54R, G98A, Y103F | S56R, L96I | 147 | 160 | 2.05 | 0.92 |
| AHF40569 | T31L, Y32W, G54R, G98A, | N32R, S56R, S93F, | 243 | 160 | 13.9 | 1.52 |
| | Y103F | L96I | | | | |
| AHF40570 | G54R, G98A, Y103F | S56R, L96I | 124 | 160 | 2.87 | 0.78 |
| AHF40571 | G98A, Y103F | N32R, S56R, L96I | 232 | 160 | 16.1 | 1.45 |
| AHF40572 | G54R, G98A, Y103F | N32R, L96I | 232 | 160 | 18.6 | 1.45 |
| Negative | — | — | — | — | 2.6 | |
| control | | | | | | |
| Blank | — | — | — | — | 1.9 | |

TABLE 20
Exemplary TCRβV6-5-binding Domains

| | | | Kabat CDRs | | | | | | IMGT CDRs | | | | | |
|---|---|---|---|---|---|---|---|---|---|---|---|---|---|---|
| ID | VH | VL | CDRL1 | CDRL2 | CDRL3 | CDRH1 | CDRH2 | CDRH3 | CDRL1 | CDRL2 | CDRL3 | CDRH1 | CDRH2 | CDRH3 |
| Wildtype | 67 | 66 | 71 | 72 | 73 | 112 | 69 | 70 | 113 | SSS | 73 | 114 | 115 | 116 |
| AHF10001 | 67 | 94 | 71 | 72 | 73 | 112 | 69 | 70 | 113 | SSS | 73 | 114 | 115 | 116 |
| AHF10002 | 80 | 95 | 117 | 72 | 73 | 112 | 118 | 70 | 119 | SSS | 73 | 114 | 120 | 116 |
| AHF10003 | 81 | 96 | 71 | 72 | 73 | 112 | 121 | 70 | 113 | SSS | 73 | 114 | 122 | 116 |
| AHF10004 | 82 | 97 | 117 | 72 | 73 | 112 | 121 | 70 | 119 | SSS | 73 | 114 | 122 | 123 |
| AHF10005 | 83 | 97 | 124 | 72 | 73 | 112 | 121 | 70 | 119 | SSS | 73 | 114 | 122 | 123 |
| AHF10006 | 67 | 98 | 71 | 72 | 73 | 126 | 69 | 70 | 113 | SSS | 73 | 125 | 115 | 116 |
| AHF10007 | 84 | 99 | 127 | 128 | 73 | 126 | 129 | 70 | 130 | SSS | 73 | 131 | 132 | 133 |
| AHF40565 | 85 | 100 | 134 | 72 | 135 | 136 | 69 | 137 | 138 | SSS | 135 | 139 | 115 | 140 |
| AHF40566 | 86 | 101 | 134 | 72 | 141 | 112 | 142 | 137 | 138 | SSS | 141 | 114 | 143 | 140 |
| AHF40571 | 87 | 102 | 134 | 144 | 135 | 112 | 69 | 137 | 138 | SSS | 135 | 114 | 115 | 140 |
| AHF40572 | 85 | 101 | 134 | 72 | 135 | 112 | 142 | 137 | 138 | SSS | 135 | 114 | 143 | 140 |
| AHF40557 | 85 | 103 | 134 | 72 | 135 | 147 | 69 | 137 | 138 | SSS | 135 | 148 | 115 | 140 |
| AHF40562 | 88 | 104 | 71 | 128 | 135 | 147 | 142 | 137 | 113 | SSS | 135 | 148 | 143 | 140 |
| AHF40567 | 89 | 105 | 134 | 128 | 135 | 136 | 142 | 137 | 138 | SSS | 135 | 139 | 143 | 140 |
| AHF40570 | 91 | 101 | 71 | 144 | 135 | 112 | 142 | 137 | 113 | SSS | 135 | 114 | 143 | 140 |
| AHF40553 | 87 | 106 | 134 | 144 | 135 | 112 | 142 | 137 | 138 | SSS | 135 | 114 | 143 | 140 |
| AHF40555 | 87 | 107 | 134 | 144 | 135 | 136 | 142 | 137 | 138 | SSS | 135 | 139 | 143 | 140 |
| AHF40556 | 89 | 108 | 134 | 128 | 141 | 145 | 69 | 137 | 138 | SSS | 141 | 146 | 115 | 140 |
| AHF40559 | 90 | 109 | 71 | 144 | 141 | 147 | 69 | 137 | 138 | SSS | 141 | 148 | 115 | 140 |
| AHF40560 | 86 | 109 | 134 | 72 | 135 | 147 | 69 | 137 | 138 | SSS | 135 | 148 | 115 | 140 |
| AHF40561 | 92 | 106 | 134 | 128 | 141 | 112 | 142 | 137 | 138 | SSS | 141 | 114 | 143 | 140 |
| AHF40563 | 92 | 110 | 134 | 128 | 141 | 136 | 69 | 137 | 138 | SSS | 141 | 139 | 115 | 140 |
| AHF40564 | 87 | 111 | 134 | 144 | 135 | 147 | 142 | 137 | 138 | SSS | 135 | 148 | 143 | 140 |
| AHF40568 | 91 | 107 | 71 | 144 | 135 | 136 | 142 | 137 | 138 | SSS | 135 | 139 | 143 | 140 |
| AHF40569 | 93 | 111 | 134 | 144 | 141 | 147 | 142 | 137 | 138 | SSS | 141 | 148 | 143 | 140 |

TABLE 21
Exemplary TCRβV6-5 scFv Polypeptide Molecules

| ID | SEQ ID NO |
|---|---|
| CLY-134 | 149 |
| CLY-135 | 150 |
| CLY-136 | 151 |
| CLY-137 | 152 |
| CLY-138 | 153 |
| CLY-139 | 154 |
| CLY-140 | 155 |
| CLY-141 | 156 |
| CLY-142 | 157 |
| CLY-143 | 158 |
| CLY-144 | 159 |
| CLY-145 | 160 |
| CLY-146 | 161 |
| CLY-147 | 162 |
| CLY-148 | 163 |
| CLY-149 | 164 |
| CLY-150 | 165 |
| CLY-151 | 166 |
| CLY-152 | 167 |
| CLY-153 | 168 |
| CLY-154 | 169 |
| CLY-155 | 170 |
| CLY-156 | 171 |
| CLY-157 | 172 |
| CLY-158 | 173 |
| CLY-159 | 174 |
| CLY-160 | 175 |
| CLY-161 | 176 |
| CLY-162 | 177 |
| CLY-163 | 178 |
| CLY-164 | 179 |
| CLY-165 | 180 |
| CLY-166 | 181 |
| CLY-167 | 182 |
| CLY-168 | 183 |
| CLY-169 | 184 |
| CLY-170 | 185 |
| CLY-171 | 186 |
| CLY-172 | 187 |
| CLY-173 | 188 |
| CLY-174 | 189 |
| CLY-175 | 190 |
| CLY-176 | 191 |
| CLY-177 | 192 |
| CLY-178 | 193 |
| CLY-179 | 194 |
| CLY-180 | 195 |
| CLY-181 | 196 |
| CLY-182 | 197 |
| CLY-183 | 198 |
| CLY-184 | 199 |
| CLY-185 | 200 |
| CLY-186 | 201 |
| CLY-187 | 202 |
| CLY-188 | 203 |
| CLY-189 | 204 |
| CLY-190 | 205 |
| CLY-191 | 206 |
| CLY-192 | 207 |
| CLY-193 | 208 |
| CLY-194 | 209 |
| CLY-195 | 210 |
| CLY-196 | 211 |
| CLY-197 | 212 |
| CLY-198 | 213 |
| CLY-199 | 214 |
| CLY-200 | 215 |
| CLY-201 | 216 |
| CLY-202 | 217 |
| CLY-203 | 218 |
| CLY-204 | 219 |
| CLY-205 | 220 |
| CLY-206 | 221 |
| CLY-207 | 222 |

TABLE 22
Exemplary TCRβV6-5 Fab Polypeptide Molecules

SEQ ID NO

| ID | LC | HC |
|---|---|---|
| CLY-1065 | 230 | 575 |
| CLY-008 | 231 | 576 |
| CLY-008-2 | 231 | 577 |
| CLY-008-3 | 232 | 578 |
| CLY-008-4 | 233 | 579 |
| CLY-008-5 | 234 | 580 |
| CLY-008-6 | 235 | 580 |
| CLY-008-7 | 231 | 581 |
| CLY-008-8 | 230 | 575 |
| CLY-010 | 236 | 582 |
| CLY-011 | 237 | 583 |
| CLY-012 | 238 | 584 |
| CLY-013 | 236 | 583 |
| CLY-014 | 236 | 585 |
| CLY-015 | 239 | 586 |
| CLY-016 | 240 | 587 |
| CLY-017 | 241 | 583 |
| CLY-208 | 238 | 588 |
| CLY-209 | 238 | 589 |
| CLY-210 | 240 | 590 |
| CLY-211 | 242 | 591 |
| CLY-212 | 237 | 591 |
| CLY-213 | 243 | 588 |
| CLY-214 | 243 | 592 |
| CLY-215 | 238 | 593 |
| CLY-216 | 241 | 589 |
| CLY-217 | 244 | 593 |
| CLY-218 | 245 | 576 |
| CLY-219 | 246 | 576 |
| CLY-220 | 247 | 576 |
| CLY-221 | 248 | 576 |
| CLY-222 | 249 | 576 |
| CLY-223 | 250 | 576 |
| CLY-224 | 251 | 576 |
| CLY-225 | 252 | 576 |
| CLY-226 | 253 | 576 |
| CLY-227 | 231 | 576 |
| CLY-228 | 254 | 576 |
| CLY-229 | 255 | 576 |
| CLY-230 | 256 | 576 |
| CLY-231 | 257 | 576 |
| CLY-232 | 258 | 576 |
| CLY-233 | 259 | 576 |
| CLY-234 | 260 | 576 |
| CLY-235 | 261 | 576 |
| CLY-236 | 262 | 576 |
| CLY-237 | 263 | 576 |
| CLY-238 | 264 | 576 |
| CLY-239 | 265 | 576 |
| CLY-240 | 266 | 576 |
| CLY-241 | 267 | 576 |
| CLY-242 | 268 | 576 |
| CLY-243 | 269 | 576 |
| CLY-244 | 270 | 576 |
| CLY-245 | 271 | 576 |
| CLY-246 | 272 | 576 |
| CLY-247 | 273 | 576 |
| CLY-248 | 274 | 576 |
| CLY-249 | 274 | 576 |
| CLY-250 | 274 | 576 |
| CLY-251 | 274 | 576 |
| CLY-252 | 274 | 576 |
| CLY-253 | 274 | 576 |
| CLY-254 | 274 | 576 |
| CLY-255 | 274 | 576 |
| CLY-256 | 274 | 576 |
| CLY-257 | 274 | 576 |
| CLY-258 | 275 | 576 |
| CLY-259 | 276 | 576 |
| CLY-260 | 277 | 576 |
| CLY-261 | 278 | 576 |
| CLY-262 | 279 | 576 |
| CLY-263 | 280 | 576 |
| CLY-264 | 281 | 576 |
| CLY-265 | 282 | 576 |
| CLY-266 | 283 | 576 |
| CLY-267 | 284 | 576 |
| CLY-268 | 285 | 576 |
| CLY-269 | 286 | 576 |
| CLY-270 | 287 | 576 |
| CLY-271 | 288 | 576 |
| CLY-272 | 289 | 576 |
| CLY-273 | 290 | 576 |
| CLY-274 | 291 | 576 |
| CLY-275 | 292 | 576 |
| CLY-276 | 293 | 576 |
| CLY-277 | 294 | 576 |
| CLY-278 | 295 | 576 |
| CLY-279 | 296 | 576 |
| CLY-280 | 297 | 576 |
| CLY-281 | 298 | 576 |
| CLY-282 | 299 | 576 |
| CLY-283 | 300 | 576 |
| CLY-284 | 301 | 576 |
| CLY-285 | 302 | 576 |
| CLY-286 | 303 | 576 |
| CLY-287 | 304 | 576 |
| CLY-288 | 305 | 576 |
| CLY-289 | 306 | 576 |
| CLY-290 | 307 | 576 |
| CLY-291 | 308 | 576 |
| CLY-292 | 309 | 576 |
| CLY-293 | 310 | 576 |
| CLY-294 | 311 | 576 |
| CLY-295 | 312 | 576 |
| CLY-296 | 313 | 576 |
| CLY-297 | 314 | 576 |
| CLY-298 | 315 | 576 |
| CLY-299 | 316 | 576 |
| CLY-300 | 317 | 576 |
| CLY-301 | 318 | 576 |
| CLY-302 | 319 | 576 |
| CLY-303 | 320 | 576 |
| CLY-304 | 321 | 576 |
| CLY-305 | 322 | 576 |
| CLY-306 | 323 | 576 |
| CLY-307 | 324 | 576 |
| CLY-308 | 325 | 576 |
| CLY-309 | 326 | 576 |
| CLY-310 | 327 | 576 |
| CLY-311 | 328 | 576 |
| CLY-312 | 329 | 576 |
| CLY-313 | 330 | 576 |
| CLY-314 | 331 | 576 |
| CLY-315 | 332 | 576 |
| CLY-316 | 333 | 576 |
| CLY-317 | 334 | 576 |
| CLY-318 | 335 | 576 |
| CLY-319 | 336 | 576 |
| CLY-320 | 336 | 576 |
| CLY-321 | 337 | 576 |
| CLY-322 | 338 | 576 |
| CLY-323 | 339 | 576 |
| CLY-324 | 340 | 576 |
| CLY-325 | 341 | 576 |
| CLY-326 | 342 | 576 |
| CLY-327 | 343 | 576 |
| CLY-328 | 344 | 576 |
| CLY-329 | 345 | 576 |
| CLY-330 | 346 | 576 |
| CLY-331 | 347 | 576 |
| CLY-332 | 348 | 576 |
| CLY-333 | 349 | 576 |
| CLY-334 | 350 | 576 |
| CLY-335 | 351 | 576 |
| CLY-336 | 352 | 576 |
| CLY-337 | 353 | 576 |
| CLY-338 | 354 | 576 |
| CLY-339 | 355 | 576 |
| CLY-340 | 356 | 576 |
| CLY-341 | 357 | 576 |
| CLY-342 | 358 | 576 |
| CLY-343 | 359 | 576 |
| CLY-344 | 360 | 576 |
| CLY-345 | 361 | 576 |
| CLY-346 | 362 | 576 |
| CLY-347 | 363 | 576 |
| CLY-348 | 364 | 576 |
| CLY-349 | 365 | 576 |
| CLY-350 | 366 | 576 |
| CLY-351 | 367 | 576 |
| CLY-352 | 368 | 576 |
| CLY-353 | 369 | 576 |
| CLY-354 | 370 | 576 |
| CLY-355 | 371 | 576 |
| CLY-356 | 372 | 576 |
| CLY-357 | 373 | 576 |
| CLY-358 | 374 | 576 |
| CLY-359 | 375 | 576 |
| CLY-360 | 376 | 576 |
| CLY-361 | 377 | 576 |
| CLY-362 | 378 | 576 |
| CLY-363 | 379 | 576 |
| CLY-364 | 380 | 576 |
| CLY-365 | 381 | 576 |
| CLY-366 | 382 | 576 |
| CLY-367 | 383 | 576 |
| CLY-368 | 384 | 576 |
| CLY-369 | 385 | 576 |
| CLY-370 | 386 | 576 |
| CLY-371 | 387 | 576 |
| CLY-372 | 388 | 576 |
| CLY-373 | 389 | 576 |
| CLY-374 | 390 | 576 |
| CLY-375 | 391 | 576 |
| CLY-376 | 392 | 576 |
| CLY-377 | 393 | 576 |
| CLY-378 | 394 | 576 |
| CLY-379 | 395 | 576 |
| CLY-380 | 396 | 576 |
| CLY-381 | 397 | 576 |
| CLY-382 | 398 | 576 |
| CLY-383 | 399 | 576 |
| CLY-384 | 400 | 576 |
| CLY-385 | 401 | 576 |
| CLY-386 | 402 | 576 |
| CLY-387 | 403 | 576 |
| CLY-388 | 404 | 576 |
| CLY-389 | 405 | 576 |
| CLY-390 | 406 | 576 |
| CLY-391 | 407 | 576 |
| CLY-392 | 408 | 576 |
| CLY-393 | 409 | 576 |
| CLY-394 | 410 | 576 |
| CLY-395 | 411 | 576 |
| CLY-396 | 412 | 576 |
| CLY-397 | 413 | 576 |
| CLY-398 | 414 | 576 |
| CLY-399 | 415 | 576 |
| CLY-400 | 416 | 576 |
| CLY-401 | 417 | 576 |
| CLY-402 | 418 | 576 |
| CLY-403 | 419 | 576 |
| CLY-404 | 420 | 576 |
| CLY-405 | 421 | 576 |
| CLY-406 | 422 | 576 |
| CLY-407 | 423 | 576 |
| CLY-408 | 424 | 576 |
| CLY-409 | 425 | 576 |
| CLY-410 | 426 | 576 |
| CLY-411 | 427 | 576 |
| CLY-412 | 428 | 576 |
| CLY-413 | 429 | 576 |
| CLY-414 | 430 | 576 |
| CLY-415 | 431 | 576 |
| CLY-416 | 432 | 576 |
| CLY-417 | 433 | 576 |
| CLY-418 | 434 | 576 |
| CLY-419 | 435 | 576 |
| CLY-420 | 436 | 576 |
| CLY-421 | 437 | 576 |
| CLY-422 | 438 | 576 |
| CLY-423 | 439 | 576 |
| CLY-424 | 440 | 576 |
| CLY-425 | 441 | 576 |
| CLY-426 | 442 | 576 |
| CLY-427 | 443 | 576 |
| CLY-428 | 444 | 576 |
| CLY-429 | 445 | 576 |
| CLY-430 | 446 | 576 |
| CLY-431 | 447 | 576 |
| CLY-432 | 448 | 576 |
| CLY-433 | 449 | 576 |
| CLY-434 | 450 | 576 |
| CLY-435 | 451 | 576 |
| CLY-436 | 452 | 576 |
| CLY-437 | 453 | 576 |
| CLY-438 | 454 | 576 |
| CLY-439 | 455 | 576 |
| CLY-440 | 456 | 576 |
| CLY-441 | 231 | 576 |
| CLY-442 | 457 | 576 |
| CLY-443 | 458 | 576 |
| CLY-444 | 459 | 576 |
| CLY-445 | 460 | 576 |
| CLY-446 | 461 | 576 |
| CLY-447 | 462 | 576 |
| CLY-448 | 463 | 576 |
| CLY-449 | 464 | 576 |
| CLY-450 | 465 | 576 |
| CLY-451 | 466 | 576 |
| CLY-452 | 467 | 576 |
| CLY-453 | 468 | 576 |
| CLY-454 | 469 | 576 |
| CLY-455 | 470 | 576 |
| CLY-456 | 471 | 576 |
| CLY-457 | 472 | 576 |
| CLY-458 | 473 | 576 |
| CLY-459 | 474 | 576 |
| CLY-460 | 475 | 576 |
| CLY-461 | 476 | 576 |
| CLY-462 | 477 | 576 |
| CLY-463 | 478 | 576 |
| CLY-464 | 479 | 576 |
| CLY-465 | 480 | 576 |
| CLY-466 | 481 | 576 |
| CLY-467 | 482 | 576 |
| CLY-468 | 483 | 576 |
| CLY-469 | 484 | 576 |
| CLY-470 | 485 | 576 |
| CLY-471 | 231 | 576 |
| CLY-472 | 486 | 576 |
| CLY-473 | 487 | 576 |
| CLY-474 | 488 | 576 |
| CLY-475 | 489 | 576 |
| CLY-476 | 490 | 576 |
| CLY-477 | 491 | 576 |
| CLY-478 | 492 | 576 |
| CLY-479 | 493 | 576 |
| CLY-480 | 494 | 576 |
| CLY-481 | 495 | 576 |
| CLY-482 | 496 | 576 |
| CLY-483 | 497 | 576 |
| CLY-484 | 498 | 576 |
| CLY-485 | 499 | 576 |
| CLY-486 | 500 | 576 |
| CLY-487 | 501 | 576 |
| CLY-488 | 502 | 576 |
| CLY-489 | 503 | 576 |
| CLY-490 | 504 | 576 |
| CLY-491 | 505 | 576 |
| CLY-492 | 506 | 576 |
| CLY-493 | 507 | 576 |
| CLY-494 | 508 | 576 |
| CLY-495 | 509 | 576 |
| CLY-496 | 510 | 576 |
| CLY-497 | 511 | 576 |
| CLY-498 | 512 | 576 |
| CLY-499 | 513 | 576 |
| CLY-500 | 514 | 576 |
| CLY-501 | 515 | 576 |
| CLY-502 | 516 | 576 |
| CLY-503 | 517 | 576 |
| CLY-504 | 518 | 576 |
| CLY-505 | 519 | 576 |
| CLY-506 | 520 | 576 |
| CLY-507 | 521 | 576 |
| CLY-508 | 522 | 576 |
| CLY-509 | 523 | 576 |
| CLY-510 | 524 | 576 |
| CLY-511 | 525 | 576 |
| CLY-512 | 526 | 576 |
| CLY-513 | 527 | 576 |
| CLY-514 | 528 | 576 |
| CLY-515 | 529 | 576 |
| CLY-516 | 530 | 576 |
| CLY-517 | 531 | 576 |
| CLY-518 | 532 | 576 |
| CLY-519 | 533 | 576 |
| CLY-520 | 534 | 576 |
| CLY-521 | 535 | 576 |
| CLY-522 | 231 | 576 |
| CLY-523 | 536 | 576 |
| CLY-524 | 537 | 576 |
| CLY-525 | 538 | 576 |
| CLY-526 | 539 | 576 |
| CLY-527 | 540 | 576 |
| CLY-528 | 541 | 576 |
| CLY-529 | 542 | 576 |
| CLY-530 | 543 | 576 |
| CLY-531 | 544 | 576 |
| CLY-532 | 545 | 576 |
| CLY-533 | 546 | 576 |
| CLY-534 | 547 | 576 |
| CLY-535 | 548 | 576 |
| CLY-536 | 549 | 576 |
| CLY-537 | 550 | 576 |
| CLY-538 | 551 | 576 |
| CLY-539 | 552 | 576 |
| CLY-540 | 553 | 576 |
| CLY-541 | 554 | 576 |
| CLY-542 | 555 | 576 |
| CLY-543 | 556 | 576 |
| CLY-544 | 557 | 576 |
| CLY-545 | 558 | 576 |
| CLY-546 | 559 | 576 |
| CLY-547 | 560 | 576 |
| CLY-548 | 561 | 576 |
| CLY-549 | 562 | 576 |
| CLY-550 | 563 | 576 |
| CLY-551 | 564 | 576 |
| CLY-552 | 565 | 576 |
| CLY-553 | 566 | 576 |
| CLY-554 | 567 | 576 |
| CLY-555 | 568 | 576 |
| CLY-556 | 569 | 576 |
| CLY-557 | 570 | 576 |
| CLY-558 | 571 | 576 |
| CLY-559 | 572 | 576 |
| CLY-560 | 231 | 576 |
| CLY-561 | 573 | 576 |
| CLY-562 | 574 | 576 |
| CLY-563 | 231 | 594 |
| CLY-564 | 231 | 595 |
| CLY-565 | 231 | 596 |
| CLY-566 | 231 | 597 |
| CLY-567 | 231 | 598 |
| CLY-568 | 231 | 599 |
| CLY-569 | 231 | 600 |
| CLY-570 | 231 | 601 |
| CLY-571 | 231 | 602 |
| CLY-572 | 231 | 603 |
| CLY-573 | 231 | 604 |
| CLY-574 | 231 | 605 |
| CLY-575 | 231 | 606 |
| CLY-576 | 231 | 607 |
| CLY-577 | 231 | 608 |
| CLY-578 | 231 | 609 |
| CLY-579 | 231 | 610 |
| CLY-580 | 231 | 611 |
| CLY-581 | 231 | 612 |
| CLY-582 | 231 | 613 |
| CLY-583 | 231 | 614 |
| CLY-584 | 231 | 615 |
| CLY-585 | 231 | 616 |
| CLY-586 | 231 | 617 |
| CLY-587 | 231 | 618 |
| CLY-588 | 231 | 619 |
| CLY-589 | 231 | 620 |
| CLY-590 | 231 | 621 |
| CLY-591 | 231 | 622 |
| CLY-592 | 231 | 623 |
| CLY-593 | 231 | 624 |
| CLY-594 | 231 | 625 |
| CLY-595 | 231 | 626 |
| CLY-596 | 231 | 627 |
| CLY-597 | 231 | 628 |
| CLY-598 | 231 | 629 |
| CLY-599 | 231 | 630 |
| CLY-600 | 231 | 631 |
| CLY-601 | 231 | 632 |
| CLY-602 | 231 | 633 |
| CLY-603 | 231 | 634 |
| CLY-604 | 231 | 635 |
| CLY-605 | 231 | 636 |
| CLY-606 | 231 | 637 |
| CLY-607 | 231 | 638 |
| CLY-608 | 231 | 639 |
| CLY-609 | 231 | 640 |
| CLY-610 | 231 | 641 |
| CLY-611 | 231 | 642 |
| CLY-612 | 231 | 643 |
| CLY-613 | 231 | 644 |
| CLY-614 | 231 | 645 |
| CLY-615 | 231 | 646 |
| CLY-616 | 231 | 647 |
| CLY-617 | 231 | 648 |
| CLY-618 | 231 | 649 |
| CLY-619 | 231 | 650 |
| CLY-620 | 231 | 651 |
| CLY-621 | 231 | 652 |
| CLY-622 | 231 | 653 |
| CLY-623 | 231 | 654 |
| CLY-624 | 231 | 655 |
| CLY-625 | 231 | 656 |
| CLY-626 | 231 | 657 |
| CLY-627 | 231 | 658 |
| CLY-628 | 231 | 659 |
| CLY-629 | 231 | 660 |
| CLY-630 | 231 | 661 |
| CLY-631 | 231 | 662 |
| CLY-632 | 231 | 663 |
| CLY-624 | 231 | 655 |
| CLY-633 | 231 | 664 |
| CLY-634 | 231 | 665 |
| CLY-635 | 231 | 666 |
| CLY-636 | 231 | 667 |
| CLY-637 | 231 | 668 |
| CLY-638 | 231 | 669 |
| CLY-639 | 231 | 670 |
| CLY-640 | 231 | 671 |
| CLY-641 | 231 | 672 |
| CLY-642 | 231 | 673 |
| CLY-643 | 231 | 576 |
| CLY-644 | 231 | 674 |
| CLY-645 | 231 | 675 |
| CLY-646 | 231 | 676 |
| CLY-647 | 231 | 677 |
| CLY-648 | 231 | 678 |
| CLY-649 | 231 | 679 |
| CLY-650 | 231 | 680 |
| CLY-651 | 231 | 681 |
| CLY-652 | 231 | 682 |
| CLY-653 | 231 | 683 |
| CLY-654 | 231 | 684 |
| CLY-655 | 231 | 685 |
| CLY-656 | 231 | 686 |
| CLY-657 | 231 | 687 |
| CLY-658 | 231 | 688 |
| CLY-659 | 231 | 689 |
| CLY-660 | 231 | 690 |
| CLY-661 | 231 | 691 |
| CLY-662 | 231 | 692 |
| CLY-663 | 231 | 693 |
| CLY-664 | 231 | 694 |
| CLY-665 | 231 | 695 |
| CLY-666 | 231 | 696 |
| CLY-667 | 231 | 697 |
| CLY-668 | 231 | 698 |
| CLY-669 | 231 | 699 |
| CLY-670 | 231 | 700 |
| CLY-671 | 231 | 701 |
| CLY-672 | 231 | 702 |
| CLY-673 | 231 | 703 |
| CLY-674 | 231 | 704 |
| CLY-675 | 231 | 705 |
| CLY-676 | 231 | 706 |
| CLY-677 | 231 | 707 |
| CLY-678 | 231 | 708 |
| CLY-679 | 231 | 709 |
| CLY-680 | 231 | 710 |
| CLY-681 | 231 | 711 |
| CLY-682 | 231 | 712 |
| CLY-683 | 231 | 713 |
| CLY-684 | 231 | 714 |
| CLY-685 | 231 | 715 |
| CLY-686 | 231 | 716 |
| CLY-687 | 231 | 717 |
| CLY-688 | 231 | 718 |
| CLY-689 | 231 | 719 |
| CLY-690 | 231 | 720 |
| CLY-691 | 231 | 721 |
| CLY-692 | 231 | 722 |
| CLY-693 | 231 | 723 |
| CLY-694 | 231 | 724 |
| CLY-695 | 231 | 725 |
| CLY-696 | 231 | 726 |
| CLY-697 | 231 | 727 |
| CLY-698 | 231 | 728 |
| CLY-699 | 231 | 729 |
| CLY-700 | 231 | 730 |
| CLY-701 | 231 | 731 |
| CLY-702 | 231 | 732 |
| CLY-703 | 231 | 733 |
| CLY-704 | 231 | 734 |
| CLY-705 | 23 | 735 |
| CLY-706 | 231 | 736 |
| CLY-707 | 231 | 737 |
| CLY-708 | 231 | 738 |
| CLY-709 | 231 | 739 |
| CLY-710 | 231 | 740 |
| CLY-711 | 231 | 741 |
| CLY-712 | 231 | 742 |
| CLY-713 | 231 | 743 |
| CLY-714 | 231 | 744 |
| CLY-715 | 231 | 745 |
| CLY-716 | 231 | 746 |
| CLY-717 | 231 | 747 |
| CLY-718 | 231 | 748 |
| CLY-719 | 231 | 749 |
| CLY-720 | 231 | 750 |
| CLY-721 | 231 | 751 |
| CLY-722 | 231 | 752 |
| CLY-723 | 231 | 753 |
| CLY-724 | 231 | 754 |
| CLY-725 | 231 | 755 |
| CLY-726 | 231 | 756 |
| CLY-727 | 231 | 757 |
| CLY-728 | 231 | 758 |
| CLY-729 | 231 | 759 |
| CLY-730 | 231 | 760 |
| CLY-731 | 231 | 761 |
| CLY-732 | 231 | 762 |
| CLY-733 | 231 | 763 |
| CLY-734 | 231 | 764 |
| CLY-735 | 231 | 765 |
| CLY-736 | 231 | 766 |
| CLY-737 | 231 | 767 |
| CLY-738 | 231 | 768 |
| CLY-739 | 231 | 769 |
| CLY-740 | 231 | 770 |
| CLY-741 | 231 | 771 |
| CLY-742 | 231 | 772 |
| CLY-743 | 231 | 773 |
| CLY-744 | 231 | 774 |
| CLY-745 | 231 | 775 |
| CLY-746 | 231 | 776 |
| CLY-747 | 231 | 777 |
| CLY-748 | 231 | 778 |
| CLY-749 | 231 | 779 |
| CLY-750 | 231 | 780 |
| CLY-751 | 231 | 781 |
| CLY-752 | 231 | 782 |
| CLY-753 | 231 | 783 |
| CLY-754 | 231 | 784 |
| CLY-755 | 23 | 785 |
| CLY-756 | 231 | 786 |
| CLY-757 | 231 | 787 |
| CLY-758 | 231 | 788 |
| CLY-759 | 23 | 789 |
| CLY-760 | 231 | 790 |
| CLY-761 | 231 | 791 |
| CLY-762 | 231 | 792 |
| CLY-763 | 231 | 793 |
| CLY-764 | 231 | 794 |
| CLY-765 | 231 | 795 |
| CLY-766 | 231 | 796 |
| CLY-767 | 231 | 797 |
| CLY-768 | 231 | 798 |
| CLY-769 | 231 | 799 |
| CLY-770 | 231 | 800 |
| CLY-771 | 231 | 801 |
| CLY-772 | 231 | 802 |
| CLY-773 | 23 | 803 |
| CLY-774 | 231 | 804 |
| CLY-775 | 231 | 805 |
| CLY-776 | 23 | 806 |
| CLY-777 | 231 | 807 |
| CLY-778 | 231 | 808 |
| CLY-779 | 231 | 809 |
| CLY-780 | 231 | 810 |
| CLY-781 | 231 | 811 |
| CLY-782 | 231 | 812 |
| CLY-783 | 231 | 813 |
| CLY-784 | 231 | 814 |
| CLY-785 | 231 | 815 |
| CLY-786 | 231 | 816 |
| CLY-787 | 231 | 817 |
| CLY-788 | 231 | 818 |
| CLY-789 | 231 | 819 |
| CLY-790 | 231 | 820 |
| CLY-791 | 231 | 821 |
| CLY-792 | 231 | 822 |
| CLY-793 | 231 | 823 |
| CLY-794 | 231 | 824 |
| CLY-795 | 231 | 825 |
| CLY-796 | 231 | 826 |
| CLY-797 | 231 | 827 |
| CLY-798 | 231 | 828 |
| CLY-799 | 231 | 829 |
| CLY-800 | 231 | 830 |
| CLY-801 | 231 | 831 |
| CLY-802 | 231 | 832 |
| CLY-803 | 231 | 833 |
| CLY-804 | 231 | 834 |
| CLY-805 | 231 | 835 |
| CLY-806 | 231 | 836 |
| CLY-807 | 231 | 837 |
| CLY-808 | 231 | 838 |
| CLY-809 | 231 | 839 |
| CLY-810 | 231 | 840 |
| CLY-811 | 231 | 841 |
| CLY-812 | 231 | 842 |
| CLY-813 | 231 | 843 |
| CLY-814 | 231 | 844 |
| CLY-815 | 231 | 845 |
| CLY-816 | 231 | 846 |
| CLY-817 | 231 | 847 |
| CLY-818 | 231 | 848 |
| CLY-819 | 231 | 849 |
| CLY-820 | 231 | 850 |
| CLY-821 | 231 | 851 |
| CLY-822 | 231 | 852 |
| CLY-823 | 231 | 853 |
| CLY-824 | 231 | 854 |
| CLY-825 | 231 | 855 |
| CLY-826 | 231 | 856 |
| CLY-827 | 231 | 857 |
| CLY-828 | 231 | 858 |
| CLY-829 | 231 | 859 |
| CLY-830 | 231 | 860 |
| CLY-831 | 231 | 861 |
| CLY-832 | 231 | 862 |
| CLY-833 | 231 | 863 |
| CLY-834 | 231 | 864 |
| CLY-835 | 231 | 865 |
| CLY-836 | 231 | 866 |
| CLY-837 | 231 | 867 |
| CLY-838 | 231 | 868 |
| CLY-839 | 231 | 869 |
| CLY-840 | 231 | 870 |
| CLY-841 | 231 | 871 |
| CLY-842 | 231 | 872 |
| CLY-843 | 231 | 873 |
| CLY-844 | 231 | 874 |
| CLY-845 | 231 | 875 |
| CLY-846 | 231 | 876 |
| CLY-847 | 231 | 877 |
| CLY-848 | 231 | 878 |
| CLY-849 | 231 | 879 |
| CLY-850 | 231 | 880 |
| CLY-851 | 231 | 881 |
| CLY-852 | 231 | 882 |
| CLY-853 | 231 | 883 |
| CLY-854 | 231 | 884 |
| CLY-855 | 231 | 576 |
| CLY-856 | 231 | 885 |
| CLY-857 | 231 | 886 |
| CLY-858 | 231 | 887 |
| CLY-859 | 231 | 888 |
| CLY-860 | 231 | 889 |
| CLY-861 | 231 | 890 |
| CLY-862 | 231 | 891 |
| CLY-863 | 231 | 892 |
| CLY-864 | 231 | 893 |
| CLY-865 | 231 | 894 |
| CLY-866 | 231 | 895 |
| CLY-867 | 231 | 896 |
| CLY-868 | 231 | 897 |
| CLY-869 | 231 | 898 |
| CLY-870 | 231 | 899 |
| CLY-871 | 231 | 900 |
| CLY-872 | 231 | 901 |
| CLY-873 | 231 | 902 |
| CLY-874 | 231 | 903 |
| CLY-875 | 231 | 904 |
| CLY-876 | 231 | 905 |
| CLY-877 | 231 | 906 |
| CLY-878 | 231 | 907 |
| CLY-879 | 231 | 908 |
| CLY-880 | 231 | 909 |
| CLY-881 | 231 | 910 |
| CLY-882 | 231 | 911 |
| CLY-883 | 231 | 912 |
| CLY-884 | 231 | 913 |
| CLY-885 | 231 | 914 |
| CLY-886 | 231 | 915 |
| CLY-887 | 231 | 916 |
| CLY-888 | 231 | 917 |
| CLY-889 | 231 | 918 |
| CLY-890 | 231 | 919 |
| CLY-891 | 231 | 920 |
| CLY-892 | 231 | 921 |
| CLY-893 | 231 | 922 |
| CLY-894 | 231 | 923 |
| CLY-895 | 231 | 924 |
| CLY-896 | 231 | 925 |
| CLY-897 | 231 | 926 |
| CLY-898 | 231 | 927 |
| CLY-899 | 231 | 928 |
| CLY-900 | 231 | 929 |
| CLY-901 | 231 | 930 |
| CLY-902 | 231 | 931 |
| CLY-903 | 231 | 932 |
| CLY-904 | 231 | 933 |
| CLY-905 | 231 | 934 |
| CLY-906 | 231 | 935 |
| CLY-907 | 231 | 936 |
| CLY-908 | 231 | 937 |
| CLY-909 | 231 | 938 |
| CLY-910 | 231 | 939 |
| CLY-911 | 231 | 940 |
| CLY-912 | 231 | 941 |
| CLY-913 | 231 | 942 |
| CLY-914 | 231 | 943 |
| CLY-915 | 231 | 944 |
| CLY-916 | 231 | 945 |
| CLY-917 | 231 | 946 |
| CLY-918 | 231 | 947 |
| CLY-919 | 231 | 948 |
| CLY-920 | 231 | 949 |
| CLY-921 | 231 | 950 |
| CLY-922 | 231 | 951 |
| CLY-923 | 231 | 952 |
| CLY-924 | 231 | 953 |
| CLY-925 | 231 | 954 |
| CLY-926 | 231 | 955 |
| CLY-927 | 231 | 956 |
| CLY-928 | 231 | 957 |
| CLY-929 | 231 | 958 |
| CLY-930 | 231 | 959 |
| CLY-931 | 231 | 960 |
| CLY-932 | 231 | 961 |
| CLY-933 | 231 | 962 |
| CLY-934 | 231 | 963 |
| CLY-935 | 231 | 964 |
| CLY-936 | 231 | 965 |
| CLY-937 | 231 | 966 |
| CLY-938 | 231 | 967 |
| CLY-939 | 231 | 968 |
| CLY-940 | 231 | 969 |
| CLY-941 | 231 | 970 |
| CLY-942 | 231 | 971 |
| CLY-943 | 231 | 972 |
| CLY-944 | 231 | 973 |
| CLY-945 | 231 | 974 |
| CLY-946 | 231 | 975 |
| CLY-947 | 231 | 976 |
| CLY-948 | 231 | 977 |
| CLY-949 | 231 | 978 |
| CLY-950 | 231 | 979 |
| CLY-951 | 231 | 980 |
| CLY-952 | 231 | 981 |
| CLY-953 | 231 | 982 |
| CLY-954 | 231 | 983 |
| CLY-955 | 231 | 984 |
| CLY-956 | 231 | 985 |
| CLY-957 | 231 | 986 |
| CLY-958 | 231 | 987 |
| CLY-959 | 231 | 988 |
| CLY-960 | 231 | 989 |
| CLY-961 | 231 | 990 |
| CLY-962 | 231 | 991 |
| CLY-963 | 231 | 992 |
| CLY-964 | 231 | 993 |
| CLY-965 | 231 | 994 |
| CLY-966 | 231 | 995 |
| CLY-967 | 231 | 996 |
| CLY-968 | 231 | 997 |
| CLY-969 | 231 | 998 |
| CLY-970 | 231 | 999 |
| CLY-971 | 231 | 1000 |
| CLY-972 | 231 | 1001 |
| CLY-973 | 231 | 1002 |
| CLY-974 | 231 | 1003 |
| CLY-975 | 231 | 1004 |
| CLY-976 | 231 | 1005 |
| CLY-977 | 231 | 1006 |
| CLY-978 | 231 | 1007 |
| CLY-979 | 231 | 1008 |
| CLY-980 | 231 | 1009 |
| CLY-981 | 231 | 1010 |
| CLY-982 | 231 | 1011 |
| CLY-983 | 231 | 1012 |
| CLY-984 | 231 | 1013 |
| CLY-985 | 231 | 1014 |
| CLY-986 | 231 | 1015 |
| CLY-987 | 231 | 1016 |
| CLY-988 | 231 | 1017 |
| CLY-989 | 231 | 1018 |
| CLY-990 | 231 | 1019 |
| CLY-991 | 231 | 1020 |
| CLY-992 | 231 | 1021 |
| CLY-993 | 231 | 1022 |
| CLY-994 | 231 | 1023 |
| CLY-995 | 231 | 1024 |
| CLY-996 | 231 | 1025 |
| CLY-997 | 231 | 1026 |
| CLY-998 | 231 | 1027 |
| CLY-999 | 231 | 1028 |
| CLY-1000 | 231 | 1029 |
| CLY-1001 | 231 | 1030 |
| CLY-1002 | 231 | 1031 |
| CLY-1003 | 231 | 1032 |
| CLY-1004 | 231 | 1033 |
| CLY-1005 | 231 | 1034 |
| CLY-1006 | 231 | 1035 |
| CLY-1007 | 231 | 1036 |
| CLY-1008 | 231 | 1037 |
| CLY-1009 | 231 | 1038 |
| CLY-1010 | 231 | 1039 |
| CLY-1011 | 231 | 1040 |
| CLY-1012 | 231 | 1041 |
| CLY-1013 | 231 | 1042 |
| CLY-1014 | 231 | 1043 |
| CLY-1015 | 231 | 1044 |
| CLY-1016 | 231 | 1045 |
| CLY-1017 | 231 | 1046 |
| CLY-1018 | 231 | 1047 |
| CLY-1019 | 231 | 1048 |
| CLY-1020 | 231 | 1049 |
| CLY-1021 | 231 | 1050 |
| CLY-1022 | 231 | 1051 |
| CLY-1023 | 231 | 1052 |
| CLY-1024 | 231 | 1053 |
| CLY-1025 | 231 | 1054 |
| CLY-1026 | 231 | 1055 |
| CLY-1027 | 231 | 1056 |
| CLY-1028 | 231 | 1057 |
| CLY-1029 | 231 | 1058 |
| CLY-1030 | 231 | 1059 |
| CLY-1031 | 231 | 1060 |
| CLY-1032 | 231 | 1061 |
| CLY-1033 | 231 | 1062 |
| CLY-1034 | 231 | 1063 |
| CLY-1035 | 231 | 1064 |
| CLY-1036 | 231 | 1065 |
| CLY-1037 | 231 | 1066 |
| CLY-1038 | 231 | 1067 |
| CLY-1039 | 231 | 1068 |
| CLY-1040 | 231 | 1069 |
| CLY-1041 | 231 | 1070 |
| CLY-1042 | 231 | 1071 |
| CLY-1043 | 231 | 1072 |
| CLY-1044 | 231 | 1073 |
| CLY-1045 | 231 | 1074 |
| CLY-1046 | 231 | 1075 |
| CLY-1047 | 231 | 1076 |
| CLY-1048 | 231 | 1077 |
| CLY-1049 | 231 | 1078 |
| CLY-1050 | 231 | 1079 |
| CLY-1051 | 231 | 1080 |
| CLY-1052 | 231 | 1081 |
| CLY-1053 | 231 | 1082 |
| CLY-1054 | 231 | 1083 |
| CLY-1055 | 231 | 1084 |

### Example 3: Design and Generation of Polypeptide Molecules Comprising TCRβV12-3-Binding Domains

### A. Saturating Mutagenesis

TCRβV12-3 mutant binders were generated by systematically mutating all CDRs of the wildtype TCRβV12-3 binder disclosed in U.S. Pat. No. 5,861,155 to identify favorable mutations and combinations of mutations to discover novel and improved binders with unique CDRs.

Table 23 shows wildtype TCRβV12-3-binding domains. Saturating mutagenesis was performed on the bolded amino acid residues in Table 23.

TABLE 23
Wildtype TCRβV12-3-Binding Domain

| SEQ ID | | |
|---|---|---|
| NO | Description | Sequence |
| 1113 | VH | EVQLVESGGGLVQPGGSLRLSCAASGFTFSNFGMHWVRQAPGKGLEW |
| | | VSYISSGSSTIYYADTLKGRFTISRDNSKNTLYLQMNSLRAEDTAVYYC |
| | | ARRGEGAMDYWGQGTTVTVSS |
| 1100 | VL | DNQLTQSPSFLSASVGDRVTITCRASSSVNYIYWYQQKPGKAPKLLIYY |
| | | TSNLAPGVPSRFSGSGSGNEYTLTISSLQPEDFATYYCQQFTSSPFTFGQ |
| | | GTKLEIK |
| 1341 | VH CDR1 | GFTFSNFGMH |
| 1160 | Kabat VH | NFGMH |
| | CDR1 | |
| 1186 | IMGT VH | GFTFSNFG |
| | CDR1 | |
| 1171 | Kabat VH | YISSGSSTIYYADTLKG |
| | CDR2 | |
| 1197 | IMGT VH | ISSGSSTI |
| | CDR2 | |
| 1180 | Kabat VH | RGEGAMDY |
| | CDR3 | |
| 1201 | IMGT VH | ARRGEGAMDY |
| | CDR3 | |
| 1148 | Kabat VL | RASSSVNYIY |
| | CDR1 | |
| 1182 | IMGT VL | SSVNY |
| | CDR1 | |
| 1156 | Kabat VL | YTSNLAP |
| | CDR2 | |
| Not | IMGT VL | YTS |
| Applicable | CDR2 | |
| 1157 | Kabat VL | QQFTSSPFT |
| | CDR3 | |
| 1157 | IMGT VL | QQFTSSPFT |
| | CDR3 | |

As in Example 2, mutant full length VH or VL domains with saturating single point mutations in individual CDR positions were then assembled via high fidelity polymerase chain reaction (PCR) techniques and appropriately designed primers and terminal restriction sites. Full length mutant VH or VL domains were then cloned into the appropriate vectors designed to express and secrete Fabs from E. coli. Libraries containing all possible amino acid mutations (except Cysteine) for a single CDR amino acid position were transformed into E. coli and plated on antibiotic containing Agar plates. E. coli colonies were picked and grown in 96 deep well blocks. Crude supernatants containing secreted Fabs were screened for their ability to bind TCRβV12-3 antigen by ELISA. Clones with improved binding over wildtype Fab produced in the same way were selected for sequencing. The Fab mutants with strengthened ELISA binding signal to the TCRβV12-3 antigen were purified by affinity chromatography. Enhanced binding strength of the purified Fabs to the TCRβV12-3 antigen was confirmed by affinity ranking using Biacore analysis.

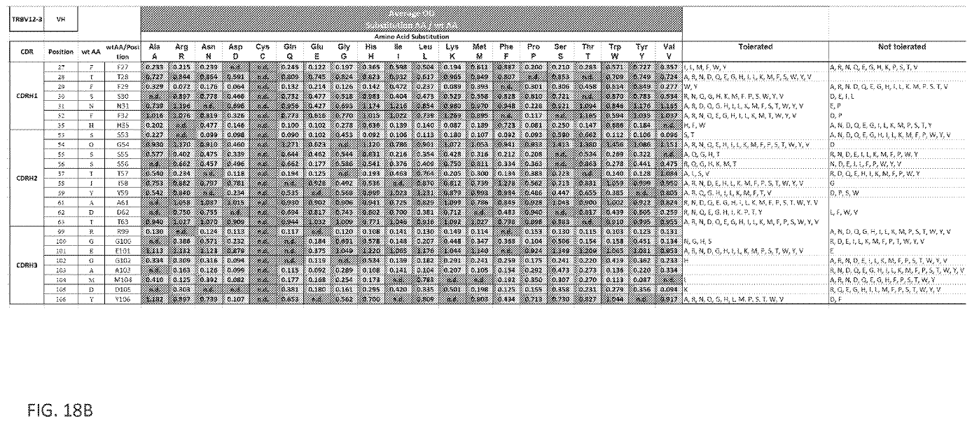

Results of the library for single point mutations on the VL mutants is provided in FIG. 18A and Table 27 and results for the single point mutations on the VH mutants is provided in FIG. 18B and Table 26. The tolerance of a single pointed mutation is determined by average OD of substitution AA/WT AA with a cutoff value of 0.5. A single pointed mutation with a value higher than 0.5 is determined to be tolerated.

Results from the Biacore analysis is provided in Table 28.

### B. Combinatorial Library Design and Screening

TCRβV12-3-binding domains were affinity matured based on the sequence activity relationships established from the single CDR mutations identified in the VH and VL sequences that strengthened binding to the TCRβV12-3 antigen. Specific mutations were selected to be combined into a combinatorial optimization library. The combinatorial library was constructed with random combinations of the preferred mutations by polymerase chain reaction (PCR). Table 24 displays the framework of the library. Table 25 below provides exemplary sequences for TCRβV12-3-binding domains.

TABLE 24
Combinatorial Mutant Library Design

| | VH | | | | | VL | | |
|---|---|---|---|---|---|---|---|---|
| CDR position | 31 | 32 | 54 | 59 | 101 | 27 | 32 | 96 |
| WT SEQ | N | F | G | Y | E | S | I | T |
| Variable mutations | I | T | T | L | A | R | A | V |
| | R | K | S | | | K | | |
| | L | Y | W | | | | | |

TABLE 25
Exemplary Sequences for TCRBV12-3-Binding Domains

| SEQ | | |
|---|---|---|
| ID | Des- | |
| NO | cription | Sequence |
| 1329 | VH CDR1 | G-B₀-B₁-B₂-B₃-B₄-B₅-G-M-B₆ |
| | Consensus | B₀: I, L, M, F, W, Y |
| | | B₁: T, A, R, N, D, Q, E, G, H, I, L, K, |
| | | M, F, S, W, Y, V |
| | | B₂: F, W, Y |
| | | B₃: R, N, Q, G, H, K, M, F, P, S, W, Y, V |
| | | B₄: N, A, R, D, Q, G, H, I, L, K, M, |
| | | F, S, T, W, Y, V |
| | | B₅: F, A, R, N, Q, E, G, H, I, L, K, |
| | | M, T, W, Y, V |
| | | B₆: H, N, F, W |
| Not | VH CDR1 | X₃₁X₃₂G₃₃M₃₄X₃₅ |
| Appli- | Kabat | X₃₁: N, R, I, Y, V, H, L, T, S, Q, D, |
| cable | | F, K, M, A, W, G, E, P |
| | | X₃₂: F, K, T, Y, V, R, A, H, I, G, M, |
| | | L, Q, N, E, W, D, P |
| | | X₃₅: H, W, F, N, G, S, D, M, A, Y, |
| | | L, T, I, E, Q, K, P |
| Not | VH CDR1 | G₂₆X₂₇X₂₈X₂₉X₃₀X₃₁X₃₂G₃₃ |
| Appli- | IMGT | X₂₇: F, Y, W, M, I, L, H, V, A, Q, G, |
| cable | | T, N, P, S, R, K, E |
| | | X₂₈: T, K, S, M, N, I, F, H, G, Q, R, |
| | | W, V, Y, E, A, D, L |
| | | X₂₉: F, Y, W, M, I, T, A, E, L, V, S, P, |
| | | N, Q, H, G, K, R, D |
| | | X₃₀: S, H, R, Q, W, F, K, N, Y, M, V, |
| | | G, P, L, E, D, I |
| | | X₃₁: N, R, I, Y, V, H, L, T, S, Q, D, |
| | | F, K, M, A, W, G, E, P |
| | | X₃₂: F, K, T, Y, V, R, A, H, I, G, M, |
| | | L, Q, N, E, W, D, P |
| 1330 | VH CDR2 | Y-I-S-B₇-B₈-B₉-B₁₀-B₁₁-B₁₂-B₁₃-Y- |
| | Consensus | B₁₄-B₁₅-B₁₆-L-K-G |
| | | B₇: S, T |
| | | B₈: G, A, R, N, Q, E, H, I, L, K, M, F, |
| | | P, S, T, W, Y, V |
| | | B₉: S, A, Q, G, H, T |
| | | B₁₀: S, R, Q, G, H, K, M, T |
| | | B₁₁: T, A, L, S, V |
| | | B₁₂: I, A, R, N, D, E, H, L, K, M, F, |
| | | P, S, T, W, Y, V |
| | | B₁₃: Y, A, R, Q, G, H, I, L, K, M, F, T, V |
| | | B₁₄: A, R, N, D, Q, E, G, H, I, L, K, M, |
| | | F, P, S, T, W, Y, V |
| | | B₁₅: D, R, N, Q, E, G, H, I, K, P, T, Y |
| | | B₁₆: T, A, R, N, D, Q, E, G, H, I, L, |
| | | K, M, F, P, S, W, Y, V |
| Not | VH CDR2 | Y₅₀I₅₁S₅₂X₅₃X₅₄X₅₅X₅₆X₅₇X₅₈X₅₉Y₆₀- |
| Appli- | Kabat | X₆₁X₆₂X₆₃L₆₄K₆₅G₆₆ |
| cable | | X₅₃: S, T, G, K, A, L, W, Y, E, D, I, |
| | | M, V, N, P, F, H, Q |
| | | X₅₄: G, W, S, Y, T, Q, V, R, H, E, M, |
| | | K, P, N, F, A, L, I, D |
| | | X₅₅: S, H, G, Q, A, T, L, M, E, W, N, |
| | | K, R, Y, D, P, I, F |
| | | X₅₆: S, M, T, P, K, R, Q, Y, L, G, N, |
| | | H, I, D, V, F, W, E |
| | | X₅₇: T, V, S, L, A, I, P, M, R, K, Q, |
| | | H, W, F, Y, E, D |
| | | X₅₈: I, F, W, Y, V, E, L, M, A, K, R, |
| | | T, D, N, S, P, H, G |
| | | X₅₉: Y, L, I, H, M, F, R, K, V, T, Q, |
| | | G, P, A, S, W, D |
| | | X₆₁: A, K, R, D, Q, W, N, S, Y, G, F, |
| | | E, P, H, T, V, I, L, M |
| | | X₆₂: D, P, T, E, H, G, R, N, Q, K, I, |
| | | Y, W, L, F, V |
| | | X₆₃: T, I, K, N, H, E, G, M, R, Y, V, |
| | | F, L, P, S, Q, A, W, D |
| Not | VH CDR2 | I₅₁S₅₂X₅₃X₅₄X₅₅X₅₆X₅₇X₅₈ |
| Appli- | IMGT | X₅₃: S, T, G, K, A, L, W, Y, E, D, I, |
| cable | | M, V, N, P, F, H, Q |
| | | X₅₄: G, W, S, Y, T, Q, V, R, H, E, M, |
| | | K, P, N, F, A, L, I, D |
| | | X₅₅: S, H, G, Q, A, T, L, M, E, W, |
| | | N, K, R, Y, D, P, I, F |
| | | X₅₆: S, M, T, P, K, R, Q, Y, L, G, |
| | | N, H, I, D, V, F, W, E |
| | | X₅₇: T, V, S, L, A, I, P, M, R, K, |
| | | Q, H, W, F, Y, E, D |
| | | X₅₈: I, F, W, Y, V, E, L, M, A, K, R, T, |
| | | D, N, S, P, H, G |
| 1331 | VH CDR3 | A-R-R-B₁₇-B₁₈-B₁₉-A-B₂₀-B₂₁-B₂₂ |
| | Consensus | B₁₇: N, G, H, S |
| | | B₁₈: E, A, R, N, D, G, H, I, L, K, M, |
| | | P, S, T, W, Y, V |
| | | B₁₉: G, H |
| | | B₂₀: M, L |
| | | B₂₁: D, K |
| | | B₂₂: Y, A, R, N, Q, G, H, L, M, P, |
| | | S, T, W, V |
| Not | VH CDR3 | X₉₉X₁₀₀X₁₀₁X₁₀₂X₁₀₃X₁₀₄X₁₀₅X₁₀₆ |
| Appli- | Kabat | X₉₉: R, I, K, L, P, N, S, Y, V, A, G, T, |
| cable | | D, Q, H, M, W |
| | | X₁₀₀: G, N, H, S, K, Y, M, R, F, L, D, |
| | | E, T, I, W, V, P |
| | | X₁₀₁: E, H, L, A, T, M, R, S, G, N, Y, |
| | | W, K, I, P, V, D |
| | | X₁₀₂: G, W, H, Y, K, R, A, F, N, V, M, |
| | | S, T, E, L, P, I, D |
| | | X₁₀₃: A, S, K, Y, G, T, V, P, R, F, W, |
| | | N, I, Q, L, H, M, D, E |
| | | X₁₀₄: M, L, A, P, F, G, E, N, T, S, R, H, |
| | | Q, W, Y, D |
| | | X₁₀₅: D, I, H, Q, Y, K, T, W, L, R, |
| | | S, E, M, G, P, F, V |
| | | X₁₀₆: Y, N, A, W, V, M, P, T, Q, R, |
| | | S, H, L, F, G, D |
| Not | VH CDR3 | A₉₇R₉₈X₉₉X₁₀₀X₁₀₁X₁₀₂X₁₀₃X₁₀₄X₁₀₅X₁₀₆ |
| Appli- | IMGT | X₉₉: R, I, K, L, P, N, S, Y, V, A, G, T, |
| cable | | D, Q, H, M, W |
| | | X₁₀₀: G, N, H, S, K, Y, M, R, F, L, D, |
| | | E, T, I, W, V, P |
| | | X₁₀₁: E, H, L, A, T, M, R, S, G, N, Y, |
| | | W, K, I, P, V, D |
| | | X₁₀₂: G, W, H, Y, K, R, A, F, N, V, M, |
| | | S, T, E, L, P, I, D |
| | | X₁₀₃: A, S, K, Y, G, T, V, P, R, F, W, |
| | | N, I, Q, L, H, M, D, E |
| | | X₁₀₄: M, L, A, P, F, G, E, N, T, S, R, |
| | | H, Q, W, Y, D |
| | | X₁₀₅: D, I, H, Q, Y, K, T, W, L, R, |
| | | S, E, M, G, P, F, V |
| | | X₁₀₆: Y, N, A, W, V, M, P, T, Q, R, S, |
| | | H, L, F, G, D |
| 1332 | VL CDR1 | R-A-J₀-J₁-J₂-J₃-J₄-J₅-J₆-Y |
| | Consensus | J₀: S, A, R, N, D, Q, G, H, I, L, M, P, |
| | | T, W, Y, V |
| | | J₁: S, A, R, N, D, Q, E, G, H, I, L, K, |
| | | M, F, P, T, W, Y, V |
| | | J₂: S, A, R, N, D, Q, E, G, H, I, K, M, |
| | | F, P, T, W, Y, V |
| | | J₃: V, I, T |
| | | J₄: N, A, R, K, M, V |
| | | J₅: R, K, F, Y |
| | | J₆: I, A, Q, L, M, S, T |
| Not | VL CDR1 | R₂₄A₂₅Z₂₆Z₂₇Z₂₈Z₂₉Z₃₀Z₃₁Z₃₂Z₃₃ |
| Appli- | Kabat | Z₂₆: S, M, V, L, Q, R, H, Y, I, T, F, A, |
| cable | | W, N, P, G, D, K |
| | | Z₂₇: S, K, R, H, Y, N, I, Q, T, G, M, L, |
| | | F, V, A, W, P, E, D |
| | | Z₂₈: S, K, N, R, P, M, Y, A, I, H, V, Q, |
| | | W, D, F, E, T, G |
| | | Z₂₉: I, T, L, A, R, M, G, P, Q, Y, F, |
| | | H, W, N, E, D |
| | | Z₃₀: N, V, R, K, M, A, T, L, W, E, Y, |
| | | Q, S, H, G, P, F, D |
| | | Z₃₁: Y, F, Y, K, R, H, A, N, M, Q, V, |
| | | P, G, L, T, E, I, D |
| | | Z₃₂: I, A, S, M, L, T, Q, D, P, H, F, |
| | | N, K, W, Y, R |
| | | Z₃₃: Y, D, W, S, L, F, H, T, R, G, P, A, N, V, I |
| Not | VL CDR1 | Z₂₇Z₂₈Z₂₉Z₃₀Z₃₁ |
| Appli- | IMGT | Z₂₇: S, K, R, H, Y, N, I, Q, T, G, M, L, |
| cable | | F, V, A, W, P, E, D |
| | | Z₂₈: S, K, N, R, P, M, Y, A, I, H, V, Q, |
| | | W, D, F, E, T, G |
| | | Z₂₉: I, T, L, A, R, M, G, P, Q, Y, F, H, |
| | | W, N, E, D |
| | | Z₃₀: N, V, R, K, M, A, T, L, W, E, Y, |
| | | Q, S, H, G, P, F, D |
| | | Z₃₁: Y, F, Y, K, R, H, A, N, M, Q, V, |
| | | P, G, L, T, E, I, D |
| 1333 | VL CDR2 | J₇-J₈-J₉-J₁₀-J₁₁-J₁₂-J₁₃ |
| | Consensus | J₇: H, L, K, M, F, T, W, Y |
| | | J₈: A, I, S, T |
| | | J₉: S, A, R, N, D, Q, E, G, H, I, L, K, |
| | | F, T, W, Y, V |
| | | J₁₀: N, A, R, D, E, G, H, I, L, K, M, |
| | | F, S, W, Y, V |
| | | J₁₁: L, A, R, N, H, K, F, P, W, V |
| | | J₁₂: A, R, Q, G, H, I, K, F, P, T, V |
| | | J₁₃: P, R, N, D, Q, E, G, H, I, L, M, F, |
| | | S, T, W, Y, V |
| Not | VL CDR2 | Z₄₉Z₅₀Z₅₁Z₅₂Z₅₃Z₅₄Z₅₅ |
| Appli- | Kabat | Z₄₉: Y, K, Y, F, I, T, H, M, W, L, Q, |
| cable | | A, G, P, V, N, E, D |
| | | Z₅₀: T, P, S, I, Q, A, W, F, M, N, G, |
| | | L, Y, R, K, D, E, H |
| | | Z₅₁: S, N, K, R, P, D, L, T, V, Y, H, |
| | | A, Q, W, I, F, C, E, G |
| | | Z₅₂: N, R, K, A, L, V, F, M, W, G, E, |
| | | Y, H, D, I, S, P |
| | | Z₅₃: L, H, K, A, N, Y, R, F, P, V, |
| | | C, I, W, G, E, D, T |
| | | Z₅₄: A, R, V, I, H, F, K, T, P, Q, N, G, E, D, Y |
| | | Z₅₅: P, R, T, Q, V, L, W, E, D, G, I, H, |
| | | S, M, F, N, Y |
| Not | VL CDR2 | Z₄₉Z₅₀Z₅₁ |
| Appli- | IMGT | Z₄₉: Y, K, Y, F, I, T, H, M, W, L, Q, A, |
| cable | | G, P, V, N, E, D |
| | | Z₅₀: T, P, S, I, Q, A, W, F, M, N, G, L, |
| | | Y, R, K, D, E, H |
| | | Z₅₁: S, N, K, R, P, D, L, T, V, Y, H, A, |
| | | Q, W, I, F, C, E, G |
| 1334 | VL CDR3 | Q-J₁₄-J₁₅-J₁₆-J₁₇-J₁₈-P-J₁₉-J₂₀ |
| | Consensus | J₁₄: Q, E |
| | | J₁₅: F, A, Q, L, K, M, S, Y |
| | | J₁₆: T, I, S |
| | | J₁₇: S, A, R, N, D, Q, G, H, I, L, K, M, |
| | | F, T, W, Y, V |
| | | J₁₈: S, A, R, N, Q, E, H, L, K, M, F, P, T, W, V |
| | | J₁₉: F, L, M, P, V |
| | | J₂₀: T, R, Q, E, H, I, L, K, M, F, S, W, Y, V |
| Not | VL CDR3 | Z₈₈Z₈₉Z₉₀Z₉₁Z₉₂Z₉₃Z₉₄Z₉₅Z₉₆ |
| Appli- | Kabat | Z₈₈: Q, E, L, Y, N, M, P, D, S, V, H, |
| cable | | T, W, I, F, R |
| | | Z₈₉: Q, H, E, K, V, R, T, G, S, A, L, I, |
| | | F, M, Y, D, P |
| | | Z₉₀: F, S, L, K, M, Y, V, Q, N, A, R, I, |
| | | T, H, G, W, D, P, E |
| | | Z₉₁: T, S, I, H, N, A, K, G, R, Q, Y, M, |
| | | F, L, V, P, W, E, D |
| | | Z₉₂: S, G, R, A, N, Y, M, W, D, T, F, |
| | | V, K, Q, H, L, I, E, P |
| | | Z₉₃: S, K, A, F, R, P, H, T, V, M, L, |
| | | E, N, Q, W, D, G |
| | | Z₉₄: P, W, E, I, Y, T, F, H, L, A, S, |
| | | C, R, K, N, Q, V |
| | | Z₉₅: F, L, P, M, V, W, A, N, S, I, G, E, H, Q, R |
| | | Z₉₆: T, V, S, H, Y, L, F, I, W, R, Q, |
| | | M, E, K, G, D |
| Not | VL CDR3 | Z₈₈Z₈₉Z₉₀Z₉₁Z₉₂Z₉₃Z₉₄Z₉₅Z₉₆ |
| Appli- | IMGT | Z₈₈: Q, E, L, Y, N, M, P, D, S, V, H, T, W, I, F, R |
| cable | | Z₈₉: Q, H, E, K, V, R, T, G, S, A, L, I, |
| | | F, M, Y, D, P |
| | | Z₉₀: F, S, L, K, M, Y, V, Q, N, A, R, I, |
| | | T, H, G, W, D, P, E |
| | | Z₉₁: T, S, I, H, N, A, K, G, R, Q, Y, M, |
| | | F, L, V, P, W, E, D |
| | | Z₉₂: S, G, R, A, N, Y, M, W, D, T, F, |
| | | V, K, Q, H, L, I, E, P |
| | | Z₉₃: S, K, A, F, R, P, H, T, V, M, L, E, |
| | | N, Q, W, D, G |
| | | Z₉₄: P, W, E, I, Y, T, F, H, L, A, S, C, |
| | | R, K, N, Q, V |
| | | Z₉₅: F, L, P, M, V, W, A, N, S, I, G, E, H, Q, R |
| | | Z₉₆: T, V, S, H, Y, L, F, I, W, R, Q, M, E, K, G, D |

The combinatorial library was transformed into E. coli, plated on antibiotic agar, colonies picked, and grown in 96 deep well blocks. Mutant Fabs were expressed and secreted into the supernatant. Crude Fab supernatants were screened by ELISA and those with stronger binding signal compared to wildtype were sequenced. Fabs were then expressed and purified by affinity chromatography before rank ordered by SPR affinity measurements using a Biacore instrument.

Employing the library described in Table 24 above, the TCRβV12-3 mutants were designed and tested using ELISA and Biacore analysis, the results of which are presented in Table 29 and Table 30, respectively.

C. Generation and Testing of Polypeptide Molecules Comprising TCRβV12-3-binding Domains

Exemplary Fabs, scFvs, and bispecific polypeptide molecules were generated to assess any potential impacts of reformatting the Fabs into different scFvs or bispecifics as well as assess any potential mammalian post translational modifications of the TCRβV12-3-binding domains that may not be present in bacterially expressed binding domains of the same sequence.

Table 31 shows exemplary TCRβV12-3-binding domains. Table 32 shows exemplary TCRβV12-3 scFvs. Table 33 shows exemplary TCRβV12-3 Fabs. The anti-TCRβV12-3 polypeptide molecules were generated in CHO cells as described in Example 2 above.

Polypeptide molecules were evaluated for their ability to bind the indicated human or cynomolgus monkey TCRβV12-3 by ELISA as described in Example 2 above. Assays utilized Rabbit anti-IgG F(ab)2-HRP at 1:2000 with a 6 minute development time. Results for the CLY-034 to CLY-037 were evaluated for binding against human TCRβV12-3 are shown at FIGS. 22I-22J and for cyno TCRβV12-3 at FIGS. 22K-22L.

TABLE 26
Heavy Chain Mutant Fab Binding to Human TCRβV12-3 by ELISA

| | Human TCRβV12-3-binding ELISA | | | Mutations in VH CDRs | | | | |
|---|---|---|---|---|---|---|---|---|
| | Mutation | WT | Ratio = | | | Point | SEQ ID NO | |
| ID | OD450 nm | OD450 nm | OD(Mutation)/OD(WT) | Chain | CDR | mutation | Fab LC | Fab HC |
| AHF33949 | 0.159 | 0.482 | 0.330 | VH | CDRH1 | F27A | 1280 | 1459 |
| AHF33948 | 0.060 | 0.482 | 0.125 | VH | CDRH1 | F27E | 1280 | 1460 |
| AHF33937 | 0.428 | 0.482 | 0.887 | VH | CDRH1 | F27F | 1280 | 1278 |
| AHF33942 | 0.140 | 0.482 | 0.291 | VH | CDRH1 | F27G | 1280 | 1461 |
| AHF33943 | 0.176 | 0.482 | 0.365 | VH | CDRH1 | F27H | 1280 | 1462 |
| AHF33962 | 0.288 | 0.482 | 0.598 | VH | CDRH1 | F27I | 1280 | 1463 |
| AHF33972 | 0.094 | 0.482 | 0.196 | VH | CDRH1 | F27K | 1280 | 1464 |
| AHF33976 | 0.272 | 0.482 | 0.564 | VH | CDRH1 | F27L | 1280 | 1465 |
| AHF33936 | 0.309 | 0.482 | 0.641 | VH | CDRH1 | F27M | 1280 | 1466 |
| AHF33969 | 0.123 | 0.482 | 0.255 | VH | CDRH1 | F27N | 1280 | 1467 |
| AHF33947 | 0.114 | 0.482 | 0.236 | VH | CDRH1 | F27P | 1280 | 1468 |
| AHF33952 | 0.152 | 0.482 | 0.315 | VH | CDRH1 | F27Q | 1280 | 1469 |
| AHF33941 | 0.104 | 0.482 | 0.215 | VH | CDRH1 | F27R | 1280 | 1470 |
| AHF33959 | 0.113 | 0.482 | 0.235 | VH | CDRH1 | F27S | 1280 | 1471 |
| AHF33960 | 0.136 | 0.482 | 0.283 | VH | CDRH1 | F27T | 1280 | 1472 |
| AHF33944 | 0.172 | 0.482 | 0.357 | VH | CDRH1 | F27V | 1280 | 1473 |
| AHF33940 | 0.365 | 0.482 | 0.757 | VH | CDRH1 | F27W | 1280 | 1474 |
| AHF33951 | 0.374 | 0.482 | 0.776 | VH | CDRH1 | F27Y | 1280 | 1475 |
| AHF33988 | 0.350 | 0.482 | 0.727 | VH | CDRH1 | T28A | 1280 | 1476 |
| AHF34003 | 0.317 | 0.482 | 0.658 | VH | CDRH1 | T28D | 1280 | 1477 |
| AHF34024 | 0.359 | 0.482 | 0.745 | VH | CDRH1 | T28E | 1280 | 1478 |
| AHF33998 | 0.452 | 0.482 | 0.938 | VH | CDRH1 | T28F | 1280 | 1479 |
| AHF34014 | 0.428 | 0.482 | 0.889 | VH | CDRH1 | T28G | 1280 | 1480 |
| AHF34006 | 0.441 | 0.482 | 0.916 | VH | CDRH1 | T28H | 1280 | 1481 |
| AHF34004 | 0.457 | 0.482 | 0.949 | VH | CDRH1 | T28I | 1280 | 1482 |
| AHF33999 | 0.510 | 0.482 | 1.059 | VH | CDRH1 | T28K | 1280 | 1483 |
| AHF34013 | 0.298 | 0.482 | 0.618 | VH | CDRH1 | T28L | 1280 | 1484 |
| AHF33983 | 0.474 | 0.482 | 0.984 | VH | CDRH1 | T28M | 1280 | 1485 |
| AHF34018 | 0.459 | 0.482 | 0.953 | VH | CDRH1 | T28N | 1280 | 1486 |
| AHF34005 | 0.421 | 0.482 | 0.875 | VH | CDRH1 | T28Q | 1280 | 1487 |
| AHF34001 | 0.406 | 0.482 | 0.844 | VH | CDRH1 | T28R | 1280 | 1488 |
| AHF34002 | 0.475 | 0.482 | 0.986 | VH | CDRH1 | T28S | 1280 | 1489 |
| AHF34017 | 0.386 | 0.482 | 0.801 | VH | CDRH1 | T28V | 1280 | 1490 |
| AHF34011 | 0.388 | 0.482 | 0.804 | VH | CDRH1 | T28W | 1280 | 1491 |
| AHF34000 | 0.375 | 0.482 | 0.778 | VH | CDRH1 | T28Y | 1280 | 1492 |
| AHF34039 | 0.218 | 0.562 | 0.387 | VH | CDRH1 | F29A | 1280 | 1493 |
| AHF34060 | 0.036 | 0.562 | 0.064 | VH | CDRH1 | F29D | 1280 | 1494 |
| AHF34037 | 0.195 | 0.562 | 0.348 | VH | CDRH1 | F29E | 1280 | 1495 |
| AHF34027 | 0.092 | 0.562 | 0.164 | VH | CDRH1 | F29G | 1280 | 1496 |
| AHF34033 | 0.093 | 0.562 | 0.166 | VH | CDRH1 | F29H | 1280 | 1497 |
| AHF34030 | 0.288 | 0.562 | 0.513 | VH | CDRH1 | F29I | 1280 | 1498 |
| AHF34066 | 0.082 | 0.562 | 0.146 | VH | CDRH1 | F29K | 1280 | 1499 |
| AHF34059 | 0.195 | 0.562 | 0.347 | VH | CDRH1 | F29L | 1280 | 1500 |
| AHF34042 | 0.294 | 0.562 | 0.523 | VH | CDRH1 | F29M | 1280 | 1501 |
| AHF34032 | 0.126 | 0.562 | 0.224 | VH | CDRH1 | F29N | 1280 | 1502 |
| AHF34053 | 0.170 | 0.562 | 0.303 | VH | CDRH1 | F29P | 1280 | 1503 |
| AHF34067 | 0.103 | 0.562 | 0.184 | VH | CDRH1 | F29Q | 1280 | 1504 |
| AHF34045 | 0.040 | 0.562 | 0.072 | VH | CDRH1 | F29R | 1280 | 1505 |
| AHF34040 | 0.172 | 0.562 | 0.306 | VH | CDRH1 | F29S | 1280 | 1506 |
| AHF34050 | 0.257 | 0.562 | 0.458 | VH | CDRH1 | F29T | 1280 | 1507 |
| AHF34056 | 0.188 | 0.562 | 0.335 | VH | CDRH1 | F29V | 1280 | 1508 |
| AHF34061 | 0.345 | 0.562 | 0.614 | VH | CDRH1 | F29W | 1280 | 1509 |
| AHF34034 | 0.529 | 0.562 | 0.941 | VH | CDRH1 | F29Y | 1280 | 1510 |
| AHF34110 | 0.290 | 0.562 | 0.516 | VH | CDRH1 | S30D | 1280 | 1511 |
| AHF34118 | 0.318 | 0.562 | 0.565 | VH | CDRH1 | S30E | 1280 | 1512 |
| AHF34111 | 0.466 | 0.562 | 0.828 | VH | CDRH1 | S30F | 1280 | 1513 |
| AHF34089 | 0.355 | 0.562 | 0.632 | VH | CDRH1 | S30G | 1280 | 1514 |
| AHF34096 | 0.579 | 0.562 | 1.029 | VH | CDRH1 | S30H | 1280 | 1515 |
| AHF34094 | 0.241 | 0.562 | 0.429 | VH | CDRH1 | S30I | 1280 | 1516 |
| AHF34083 | 0.450 | 0.562 | 0.800 | VH | CDRH1 | S30K | 1280 | 1517 |
| AHF34112 | 0.320 | 0.562 | 0.569 | VH | CDRH1 | S30L | 1280 | 1518 |
| AHF34090 | 0.383 | 0.562 | 0.681 | VH | CDRH1 | S30M | 1280 | 1519 |
| AHF34105 | 0.446 | 0.562 | 0.794 | VH | CDRH1 | S30N | 1280 | 1520 |
| AHF34114 | 0.343 | 0.562 | 0.610 | VH | CDRH1 | S30P | 1280 | 1521 |
| AHF34115 | 0.499 | 0.562 | 0.889 | VH | CDRH1 | S30Q | 1280 | 1522 |
| AHF34097 | 0.506 | 0.562 | 0.900 | VH | CDRH1 | S30R | 1280 | 1523 |
| AHF34107 | 0.405 | 0.562 | 0.721 | VH | CDRH1 | S30S | 1280 | 1278 |
| AHF34084 | 0.361 | 0.562 | 0.643 | VH | CDRH1 | S30V | 1280 | 1524 |
| AHF34104 | 0.497 | 0.562 | 0.884 | VH | CDRH1 | S30W | 1280 | 1525 |
| AHF34116 | 0.440 | 0.562 | 0.783 | VH | CDRH1 | S30Y | 1280 | 1526 |
| AHF34300 | 0.442 | 0.469 | 0.943 | VH | CDRH1 | N31A | 1280 | 1527 |
| AHF34288 | 0.481 | 0.469 | 1.026 | VH | CDRH1 | N31D | 1280 | 1528 |
| AHF34267 | 0.225 | 0.469 | 0.480 | VH | CDRH1 | N31E | 1280 | 1529 |
| AHF34286 | 0.461 | 0.469 | 0.983 | VH | CDRH1 | N31F | 1280 | 1530 |
| AHF34271 | 0.325 | 0.469 | 0.693 | VH | CDRH1 | N31G | 1280 | 1531 |
| AHF34282 | 0.553 | 0.469 | 1.180 | VH | CDRH1 | N31H | 1280 | 1532 |
| AHF34296 | 0.573 | 0.469 | 1.222 | VH | CDRH1 | N31I | 1280 | 1431 |
| AHF34287 | 0.459 | 0.469 | 0.980 | VH | CDRH1 | N31K | 1280 | 1533 |
| AHF34283 | 0.539 | 0.469 | 1.151 | VH | CDRH1 | N31L | 1280 | 1534 |
| AHF34272 | 0.454 | 0.469 | 0.970 | VH | CDRH1 | N31M | 1280 | 1535 |
| AHF34270 | 0.222 | 0.469 | 0.474 | VH | CDRH1 | N31P | 1280 | 1536 |
| AHF34291 | 0.488 | 0.469 | 1.041 | VH | CDRH1 | N31Q | 1280 | 1537 |
| AHF34278 | 0.574 | 0.469 | 1.224 | VH | CDRH1 | N31R | 1280 | 1427 |
| AHF34309 | 0.502 | 0.469 | 1.071 | VH | CDRH1 | N31S | 1280 | 1538 |
| AHF34302 | 0.524 | 0.469 | 1.118 | VH | CDRH1 | N31T | 1280 | 1539 |
| AHF34275 | 0.555 | 0.469 | 1.185 | VH | CDRH1 | N31V | 1280 | 1540 |
| AHF34312 | 0.396 | 0.469 | 0.846 | VH | CDRH1 | N31W | 1280 | 1541 |
| AHF34274 | 0.569 | 0.469 | 1.215 | VH | CDRH1 | N31Y | 1280 | 1426 |
| AHF34337 | 0.497 | 0.469 | 1.061 | VH | CDRH1 | F32A | 1280 | 1542 |
| AHF34321 | 0.193 | 0.469 | 0.413 | VH | CDRH1 | F32D | 1280 | 1543 |
| AHF34338 | 0.374 | 0.469 | 0.798 | VH | CDRH1 | F32E | 1280 | 1544 |
| AHF34328 | 0.461 | 0.469 | 0.983 | VH | CDRH1 | F32G | 1280 | 1545 |
| AHF34344 | 0.487 | 0.469 | 1.040 | VH | CDRH1 | F32H | 1280 | 1546 |
| AHF34346 | 0.479 | 0.469 | 1.022 | VH | CDRH1 | F32I | 1280 | 1547 |
| AHF34313 | 0.676 | 0.469 | 1.443 | VH | CDRH1 | F32K | 1280 | 1432 |
| AHF34355 | 0.397 | 0.469 | 0.848 | VH | CDRH1 | F32L | 1280 | 1548 |
| AHF34356 | 0.448 | 0.469 | 0.956 | VH | CDRH1 | F32M | 1280 | 1549 |
| AHF34340 | 0.384 | 0.469 | 0.819 | VH | CDRH1 | F32N | 1280 | 1550 |
| AHF34326 | 0.056 | 0.469 | 0.119 | VH | CDRH1 | F32P | 1280 | 1551 |
| AHF34334 | 0.393 | 0.469 | 0.839 | VH | CDRH1 | F32Q | 1280 | 1552 |
| AHF34327 | 0.504 | 0.469 | 1.076 | VH | CDRH1 | F32R | 1280 | 1553 |
| AHF34353 | 0.668 | 0.469 | 1.425 | VH | CDRH1 | F32T | 1280 | 1433 |
| AHF34329 | 0.506 | 0.469 | 1.080 | VH | CDRH1 | F32V | 1280 | 1554 |
| AHF34352 | 0.329 | 0.469 | 0.702 | VH | CDRH1 | F32W | 1280 | 1555 |
| AHF34318 | 0.522 | 0.469 | 1.115 | VH | CDRH1 | F32Y | 1280 | 1556 |
| AHF34388 | 0.106 | 0.452 | 0.235 | VH | CDRH1 | H35A | 1280 | 1557 |
| AHF34369 | 0.128 | 0.452 | 0.282 | VH | CDRH1 | H35D | 1280 | 1558 |
| AHF34404 | 0.050 | 0.452 | 0.111 | VH | CDRH1 | H35E | 1280 | 1559 |
| AHF34387 | 0.377 | 0.452 | 0.835 | VH | CDRH1 | H35F | 1280 | 1560 |
| AHF34365 | 0.208 | 0.452 | 0.459 | VH | CDRH1 | H35G | 1280 | 1278 |
| AHF34372 | 0.287 | 0.452 | 0.636 | VH | CDRH1 | H35H | 1280 | 1561 |
| AHF34364 | 0.066 | 0.452 | 0.147 | VH | CDRH1 | H35I | 1280 | 1562 |
| AHF34400 | 0.040 | 0.452 | 0.088 | VH | CDRH1 | H35K | 1280 | 1563 |
| AHF34360 | 0.080 | 0.452 | 0.176 | VH | CDRH1 | H35L | 1280 | 1564 |
| AHF34384 | 0.125 | 0.452 | 0.278 | VH | CDRH1 | H35M | 1280 | 1565 |
| AHF34357 | 0.240 | 0.452 | 0.531 | VH | CDRH1 | H35N | 1280 | 1566 |
| AHF34402 | 0.037 | 0.452 | 0.081 | VH | CDRH1 | H35P | 1280 | 1567 |
| AHF34393 | 0.046 | 0.452 | 0.101 | VH | CDRH1 | H35Q | 1280 | 1568 |
| AHF34386 | 0.191 | 0.452 | 0.422 | VH | CDRH1 | H35S | 1280 | 1569 |
| AHF34361 | 0.072 | 0.452 | 0.160 | VH | CDRH1 | H35T | 1280 | 1570 |
| AHF34370 | 0.415 | 0.452 | 0.920 | VH | CDRH1 | H35W | 1280 | 1571 |
| AHF34401 | 0.083 | 0.452 | 0.184 | VH | CDRH1 | H35Y | 1280 | 1278 |
| AHF34423 | 0.102 | 0.452 | 0.227 | VH | CDRH2 | S53A | 1280 | 1572 |
| AHF34444 | 0.055 | 0.452 | 0.122 | VH | CDRH2 | S53D | 1280 | 1573 |
| AHF34428 | 0.056 | 0.452 | 0.124 | VH | CDRH2 | S53E | 1280 | 1574 |
| AHF34426 | 0.042 | 0.452 | 0.092 | VH | CDRH2 | S53F | 1280 | 1575 |
| AHF34431 | 0.227 | 0.452 | 0.502 | VH | CDRH2 | S53G | 1280 | 1576 |
| AHF34427 | 0.042 | 0.452 | 0.092 | VH | CDRH2 | S53H | 1280 | 1577 |
| AHF34433 | 0.054 | 0.452 | 0.120 | VH | CDRH2 | S53I | 1280 | 1578 |
| AHF34409 | 0.122 | 0.452 | 0.269 | VH | CDRH2 | S53K | 1280 | 1579 |
| AHF34442 | 0.065 | 0.452 | 0.145 | VH | CDRH2 | S53L | 1280 | 1580 |
| AHF34437 | 0.048 | 0.452 | 0.107 | VH | CDRH2 | S53M | 1280 | 1581 |
| AHF34413 | 0.045 | 0.452 | 0.099 | VH | CDRH2 | S53N | 1280 | 1582 |
| AHF34421 | 0.044 | 0.452 | 0.096 | VH | CDRH2 | S53P | 1280 | 1583 |
| AHF34435 | 0.041 | 0.452 | 0.090 | VH | CDRH2 | S53Q | 1280 | 1584 |
| AHF34438 | 0.267 | 0.452 | 0.590 | VH | CDRH2 | S53S | 1280 | 1278 |
| AHF34434 | 0.299 | 0.452 | 0.662 | VH | CDRH2 | S53T | 1280 | 1585 |
| AHF34439 | 0.045 | 0.452 | 0.100 | VH | CDRH2 | S53V | 1280 | 1586 |
| AHF34441 | 0.063 | 0.452 | 0.138 | VH | CDRH2 | S53W | 1280 | 1587 |
| AHF34429 | 0.062 | 0.452 | 0.138 | VH | CDRH2 | S53Y | 1280 | 1588 |
| AHF34451 | 0.414 | 0.442 | 0.938 | VH | CDRH2 | G54A | 1280 | 1589 |
| AHF34460 | 0.294 | 0.442 | 0.665 | VH | CDRH2 | G54D | 1280 | 1590 |
| AHF34475 | 0.501 | 0.442 | 1.133 | VH | CDRH2 | G54E | 1280 | 1591 |
| AHF34466 | 0.421 | 0.442 | 0.952 | VH | CDRH2 | G54F | 1280 | 1592 |
| AHF34484 | 0.512 | 0.442 | 1.158 | VH | CDRH2 | G54H | 1280 | 1593 |
| AHF34456 | 0.348 | 0.442 | 0.788 | VH | CDRH2 | G54I | 1280 | 1594 |
| AHF34489 | 0.487 | 0.442 | 1.102 | VH | CDRH2 | G54K | 1280 | 1595 |
| AHF34469 | 0.405 | 0.442 | 0.917 | VH | CDRH2 | G54L | 1280 | 1596 |
| AHF34470 | 0.501 | 0.442 | 1.133 | VH | CDRH2 | G54M | 1280 | 1597 |
| AHF34465 | 0.421 | 0.442 | 0.954 | VH | CDRH2 | G54N | 1280 | 1598 |
| AHF34473 | 0.467 | 0.442 | 1.057 | VH | CDRH2 | G54P | 1280 | 1599 |
| AHF34458 | 0.576 | 0.442 | 1.304 | VH | CDRH2 | G54Q | 1280 | 1600 |
| AHF34495 | 0.527 | 0.442 | 1.194 | VH | CDRH2 | G54R | 1280 | 1601 |
| AHF34452 | 0.624 | 0.442 | 1.413 | VH | CDRH2 | G54S | 1280 | 1434 |
| AHF34491 | 0.610 | 0.442 | 1.380 | VH | CDRH2 | G54T | 1280 | 1437 |
| AHF34464 | 0.559 | 0.442 | 1.266 | VH | CDRH2 | G54V | 1280 | 1602 |
| AHF34459 | 0.643 | 0.442 | 1.456 | VH | CDRH2 | G54W | 1280 | 1435 |
| AHF34461 | 0.610 | 0.442 | 1.382 | VH | CDRH2 | G54Y | 1280 | 1436 |
| AHF34507 | 0.319 | 0.442 | 0.723 | VH | CDRH2 | S55A | 1280 | 1603 |
| AHF34531 | 0.150 | 0.442 | 0.339 | VH | CDRH2 | S55D | 1280 | 1604 |
| AHF34527 | 0.216 | 0.442 | 0.490 | VH | CDRH2 | S55E | 1280 | 1605 |
| AHF34533 | 0.094 | 0.442 | 0.212 | VH | CDRH2 | S55F | 1280 | 1606 |
| AHF34537 | 0.387 | 0.442 | 0.875 | VH | CDRH2 | S55G | 1280 | 1607 |
| AHF34523 | 0.565 | 0.442 | 1.279 | VH | CDRH2 | S55H | 1280 | 1438 |
| AHF34499 | 0.095 | 0.442 | 0.216 | VH | CDRH2 | S55I | 1280 | 1608 |
| AHF34509 | 0.202 | 0.442 | 0.458 | VH | CDRH2 | S55K | 1280 | 1609 |
| AHF34519 | 0.256 | 0.442 | 0.578 | VH | CDRH2 | S55L | 1280 | 1610 |
| AHF34497 | 0.234 | 0.442 | 0.530 | VH | CDRH2 | S55M | 1280 | 1611 |
| AHF34528 | 0.210 | 0.442 | 0.475 | VH | CDRH2 | S55N | 1280 | 1612 |
| AHF34518 | 0.119 | 0.442 | 0.268 | VH | CDRH2 | S55P | 1280 | 1613 |
| AHF34513 | 0.340 | 0.442 | 0.770 | VH | CDRH2 | S55Q | 1280 | 1614 |
| AHF34502 | 0.196 | 0.442 | 0.444 | VH | CDRH2 | S55R | 1280 | 1615 |
| AHF34521 | 0.295 | 0.442 | 0.668 | VH | CDRH2 | S55T | 1280 | 1616 |
| AHF34510 | 0.214 | 0.442 | 0.484 | VH | CDRH2 | S55W | 1280 | 1617 |
| AHF34522 | 0.179 | 0.442 | 0.405 | VH | CDRH2 | S55Y | 1280 | 1618 |
| AHF34578 | 0.230 | 0.435 | 0.529 | VH | CDRH2 | S56D | 1280 | 1619 |
| AHF34575 | 0.095 | 0.435 | 0.219 | VH | CDRH2 | S56E | 1280 | 1620 |
| AHF34567 | 0.189 | 0.435 | 0.435 | VH | CDRH2 | S56F | 1280 | 1621 |
| AHF34584 | 0.255 | 0.435 | 0.586 | VH | CDRH2 | S56G | 1280 | 1622 |
| AHF34557 | 0.235 | 0.435 | 0.541 | VH | CDRH2 | S56H | 1280 | 1623 |
| AHF34579 | 0.230 | 0.435 | 0.530 | VH | CDRH2 | S56I | 1280 | 1624 |
| AHF34548 | 0.341 | 0.435 | 0.784 | VH | CDRH2 | S56K | 1280 | 1625 |
| AHF34587 | 0.261 | 0.435 | 0.601 | VH | CDRH2 | S56L | 1280 | 1626 |
| AHF34588 | 0.408 | 0.435 | 0.938 | VH | CDRH2 | S56M | 1280 | 1627 |
| AHF34573 | 0.239 | 0.435 | 0.549 | VH | CDRH2 | S56N | 1280 | 1628 |
| AHF34550 | 0.382 | 0.435 | 0.878 | VH | CDRH2 | S56P | 1280 | 1629 |
| AHF34552 | 0.288 | 0.435 | 0.662 | VH | CDRH2 | S56Q | 1280 | 1630 |
| AHF34569 | 0.305 | 0.435 | 0.702 | VH | CDRH2 | S56R | 1280 | 1631 |
| AHF34565 | 0.398 | 0.435 | 0.916 | VH | CDRH2 | S56T | 1280 | 1632 |
| AHF34562 | 0.224 | 0.435 | 0.514 | VH | CDRH2 | S56V | 1280 | 1633 |
| AHF34543 | 0.167 | 0.435 | 0.384 | VH | CDRH2 | S56W | 1280 | 1634 |
| AHF34577 | 0.267 | 0.435 | 0.614 | VH | CDRH2 | S56Y | 1280 | 1635 |
| AHF34626 | 0.258 | 0.435 | 0.594 | VH | CDRH2 | T57A | 1280 | 1636 |
| AHF34630 | 0.053 | 0.435 | 0.123 | VH | CDRH2 | T57D | 1280 | 1637 |
| AHF34613 | 0.060 | 0.435 | 0.138 | VH | CDRH2 | T57E | 1280 | 1638 |
| AHF34620 | 0.065 | 0.435 | 0.149 | VH | CDRH2 | T57F | 1280 | 1639 |
| AHF34595 | 0.084 | 0.435 | 0.193 | VH | CDRH2 | T57H | 1280 | 1640 |
| AHF34591 | 0.202 | 0.435 | 0.464 | VH | CDRH2 | T57I | 1280 | 1641 |
| AHF34628 | 0.089 | 0.435 | 0.205 | VH | CDRH2 | T57K | 1280 | 1642 |
| AHF34607 | 0.371 | 0.435 | 0.853 | VH | CDRH2 | T57L | 1280 | 1643 |
| AHF34625 | 0.161 | 0.435 | 0.369 | VH | CDRH2 | T57M | 1280 | 1644 |
| AHF34617 | 0.195 | 0.435 | 0.448 | VH | CDRH2 | T57P | 1280 | 1645 |
| AHF34624 | 0.084 | 0.435 | 0.194 | VH | CDRH2 | T57Q | 1280 | 1646 |
| AHF34593 | 0.126 | 0.435 | 0.291 | VH | CDRH2 | T57R | 1280 | 1647 |
| AHF34612 | 0.399 | 0.435 | 0.917 | VH | CDRH2 | T57S | 1280 | 1648 |
| AHF34616 | 0.505 | 0.435 | 1.162 | VH | CDRH2 | T57V | 1280 | 1439 |
| AHF34597 | 0.065 | 0.435 | 0.150 | VH | CDRH2 | T57W | 1280 | 1649 |
| AHF34623 | 0.065 | 0.435 | 0.149 | VH | CDRH2 | T57Y | 1280 | 1650 |
| AHF34657 | 0.412 | 0.444 | 0.928 | VH | CDRH2 | I58A | 1280 | 1651 |
| AHF34673 | 0.380 | 0.444 | 0.856 | VH | CDRH2 | I58D | 1280 | 1652 |
| AHF34662 | 0.424 | 0.444 | 0.954 | VH | CDRH2 | I58E | 1280 | 1653 |
| AHF34646 | 0.568 | 0.444 | 1.278 | VH | CDRH2 | I58F | 1280 | 1440 |
| AHF34656 | 0.251 | 0.444 | 0.566 | VH | CDRH2 | I58G | 1280 | 1654 |
| AHF34668 | 0.261 | 0.444 | 0.588 | VH | CDRH2 | I58H | 1280 | 1655 |
| AHF34652 | 0.411 | 0.444 | 0.926 | VH | CDRH2 | I58K | 1280 | 1656 |
| AHF34635 | 0.423 | 0.444 | 0.951 | VH | CDRH2 | I58L | 1280 | 1657 |
| AHF34639 | 0.415 | 0.444 | 0.934 | VH | CDRH2 | I58M | 1280 | 1658 |
| AHF34678 | 0.359 | 0.444 | 0.809 | VH | CDRH2 | I58N | 1280 | 1659 |
| AHF34640 | 0.295 | 0.444 | 0.664 | VH | CDRH2 | I58P | 1280 | 1660 |
| AHF34679 | 0.407 | 0.444 | 0.916 | VH | CDRH2 | I58R | 1280 | 1661 |
| AHF34663 | 0.317 | 0.444 | 0.713 | VH | CDRH2 | I58S | 1280 | 1662 |
| AHF34667 | 0.396 | 0.444 | 0.891 | VH | CDRH2 | I58T | 1280 | 1663 |
| AHF34674 | 0.440 | 0.444 | 0.990 | VH | CDRH2 | I58V | 1280 | 1664 |
| AHF34661 | 0.470 | 0.444 | 1.059 | VH | CDRH2 | I58W | 1280 | 1665 |
| AHF34659 | 0.456 | 0.444 | 1.027 | VH | CDRH2 | I58Y | 1280 | 1666 |
| AHF34723 | 0.262 | 0.444 | 0.589 | VH | CDRH2 | Y59A | 1280 | 1667 |
| AHF34693 | 0.170 | 0.444 | 0.382 | VH | CDRH2 | Y59D | 1280 | 1668 |
| AHF34720 | 0.437 | 0.444 | 0.984 | VH | CDRH2 | Y59F | 1280 | 1669 |
| AHF34697 | 0.307 | 0.444 | 0.691 | VH | CDRH2 | Y59G | 1280 | 1670 |
| AHF34713 | 0.452 | 0.444 | 1.017 | VH | CDRH2 | Y59H | 1280 | 1671 |
| AHF34711 | 0.492 | 0.444 | 1.109 | VH | CDRH2 | Y59I | 1280 | 1672 |
| AHF34705 | 0.390 | 0.444 | 0.879 | VH | CDRH2 | Y59K | 1280 | 1673 |
| AHF34706 | 0.547 | 0.444 | 1.231 | VH | CDRH2 | Y59L | 1280 | 1441 |
| AHF34702 | 0.441 | 0.444 | 0.993 | VH | CDRH2 | Y59M | 1280 | 1674 |
| AHF34690 | 0.273 | 0.444 | 0.615 | VH | CDRH2 | Y59P | 1280 | 1675 |
| AHF34689 | 0.328 | 0.444 | 0.738 | VH | CDRH2 | Y59Q | 1280 | 1676 |
| AHF34699 | 0.427 | 0.444 | 0.961 | VH | CDRH2 | Y59R | 1280 | 1677 |
| AHF34707 | 0.207 | 0.444 | 0.467 | VH | CDRH2 | Y59S | 1280 | 1678 |
| AHF34691 | 0.332 | 0.444 | 0.748 | VH | CDRH2 | Y59T | 1280 | 1679 |
| AHF34714 | 0.380 | 0.444 | 0.856 | VH | CDRH2 | Y59V | 1280 | 1680 |
| AHF34695 | 0.191 | 0.444 | 0.431 | VH | CDRH2 | Y59W | 1280 | 1681 |
| AHF33545 | 0.508 | 0.442 | 1.149 | VH | CDRH2 | A61D | 1280 | 1682 |
| AHF33549 | 0.432 | 0.442 | 0.977 | VH | CDRH2 | A61E | 1280 | 1683 |
| AHF33569 | 0.443 | 0.442 | 1.001 | VH | CDRH2 | A61F | 1280 | 1684 |
| AHF33557 | 0.457 | 0.442 | 1.033 | VH | CDRH2 | A61G | 1280 | 1685 |
| AHF33576 | 0.416 | 0.442 | 0.941 | VH | CDRH2 | A61H | 1280 | 1686 |
| AHF33563 | 0.375 | 0.442 | 0.848 | VH | CDRH2 | A61I | 1280 | 1687 |
| AHF33544 | 0.536 | 0.442 | 1.211 | VH | CDRH2 | A61K | 1280 | 1442 |
| AHF33586 | 0.367 | 0.442 | 0.829 | VH | CDRH2 | A61L | 1280 | 1688 |
| AHF33566 | 0.348 | 0.442 | 0.786 | VH | CDRH2 | A61M | 1280 | 1689 |
| AHF33571 | 0.481 | 0.442 | 1.087 | VH | CDRH2 | A61N | 1280 | 1690 |
| AHF33562 | 0.417 | 0.442 | 0.943 | VH | CDRH2 | A61P | 1280 | 1691 |
| AHF33560 | 0.490 | 0.442 | 1.108 | VH | CDRH2 | A61Q | 1280 | 1692 |
| AHF33558 | 0.515 | 0.442 | 1.163 | VH | CDRH2 | A61R | 1280 | 1443 |
| AHF33574 | 0.474 | 0.442 | 1.071 | VH | CDRH2 | A61S | 1280 | 1693 |
| AHF33578 | 0.398 | 0.442 | 0.900 | VH | CDRH2 | A61T | 1280 | 1694 |
| AHF33546 | 0.391 | 0.442 | 0.884 | VH | CDRH2 | A61V | 1280 | 1695 |
| AHF33543 | 0.484 | 0.442 | 1.093 | VH | CDRH2 | A61W | 1280 | 1696 |
| AHF33555 | 0.471 | 0.442 | 1.064 | VH | CDRH2 | A61Y | 1280 | 1697 |
| AHF33630 | 0.402 | 0.442 | 0.909 | VH | CDRH2 | D62E | 1280 | 1698 |
| AHF33622 | 0.214 | 0.442 | 0.483 | VH | CDRH2 | D62F | 1280 | 1699 |
| AHF33598 | 0.356 | 0.442 | 0.804 | VH | CDRH2 | D62G | 1280 | 1700 |
| AHF33591 | 0.375 | 0.442 | 0.849 | VH | CDRH2 | D62H | 1280 | 1701 |
| AHF33612 | 0.310 | 0.442 | 0.700 | VH | CDRH2 | D62I | 1280 | 1702 |
| AHF33625 | 0.321 | 0.442 | 0.726 | VH | CDRH2 | D62K | 1280 | 1703 |
| AHF33626 | 0.228 | 0.442 | 0.516 | VH | CDRH2 | D62L | 1280 | 1704 |
| AHF33594 | 0.334 | 0.442 | 0.755 | VH | CDRH2 | D62N | 1280 | 1705 |
| AHF33624 | 0.484 | 0.442 | 1.095 | VH | CDRH2 | D62P | 1280 | 1706 |
| AHF33613 | 0.329 | 0.442 | 0.745 | VH | CDRH2 | D62Q | 1280 | 1707 |
| AHF33592 | 0.351 | 0.442 | 0.793 | VH | CDRH2 | D62R | 1280 | 1708 |
| AHF33606 | 0.471 | 0.442 | 1.066 | VH | CDRH2 | D62T | 1280 | 1709 |
| AHF33628 | 0.130 | 0.442 | 0.294 | VH | CDRH2 | D62V | 1280 | 1710 |
| AHF33614 | 0.256 | 0.442 | 0.578 | VH | CDRH2 | D62W | 1280 | 1711 |
| AHF33596 | 0.275 | 0.442 | 0.621 | VH | CDRH2 | D62Y | 1280 | 1712 |
| AHF34761 | 0.361 | 0.384 | 0.940 | VH | CDRH2 | T63A | 1280 | 1713 |
| AHF34769 | 0.349 | 0.384 | 0.909 | VH | CDRH2 | T63D | 1280 | 1714 |
| AHF34740 | 0.405 | 0.384 | 1.054 | VH | CDRH2 | T63E | 1280 | 1715 |
| AHF34725 | 0.388 | 0.384 | 1.009 | VH | CDRH2 | T63F | 1280 | 1716 |
| AHF34756 | 0.398 | 0.384 | 1.037 | VH | CDRH2 | T63G | 1280 | 1717 |
| AHF34760 | 0.410 | 0.384 | 1.066 | VH | CDRH2 | T63H | 1280 | 1718 |
| AHF34762 | 0.483 | 0.384 | 1.256 | VH | CDRH2 | T63I | 1280 | 1719 |
| AHF34731 | 0.456 | 0.384 | 1.185 | VH | CDRH2 | T63K | 1280 | 1444 |
| AHF34750 | 0.386 | 0.384 | 1.004 | VH | CDRH2 | T63L | 1280 | 1720 |
| AHF34733 | 0.395 | 0.384 | 1.027 | VH | CDRH2 | T63M | 1280 | 1721 |
| AHF34768 | 0.419 | 0.384 | 1.091 | VH | CDRH2 | T63N | 1280 | 1722 |
| AHF34732 | 0.385 | 0.384 | 1.001 | VH | CDRH2 | T63P | 1280 | 1723 |
| AHF34727 | 0.363 | 0.384 | 0.944 | VH | CDRH2 | T63Q | 1280 | 1724 |
| AHF34757 | 0.395 | 0.384 | 1.027 | VH | CDRH2 | T63R | 1280 | 1725 |
| AHF34771 | 0.378 | 0.384 | 0.983 | VH | CDRH2 | T63S | 1280 | 1726 |
| AHF34742 | 0.391 | 0.384 | 1.016 | VH | CDRH2 | T63V | 1280 | 1727 |
| AHF34759 | 0.359 | 0.384 | 0.935 | VH | CDRH2 | T63W | 1280 | 1728 |
| AHF34737 | 0.391 | 0.384 | 1.018 | VH | CDRH2 | T63Y | 1280 | 1729 |
| AHF34773 | 0.050 | 0.384 | 0.130 | VH | CDRH3 | R99A | 1280 | 1730 |
| AHF34774 | 0.046 | 0.384 | 0.120 | VH | CDRH3 | R99D | 1280 | 1731 |
| AHF34775 | 0.048 | 0.384 | 0.125 | VH | CDRH3 | R99G | 1280 | 1732 |
| AHF34778 | 0.045 | 0.384 | 0.118 | VH | CDRH3 | R99H | 1280 | 1733 |
| AHF34813 | 0.081 | 0.384 | 0.210 | VH | CDRH3 | R99I | 1280 | 1734 |
| AHF34802 | 0.069 | 0.384 | 0.180 | VH | CDRH3 | R99K | 1280 | 1735 |
| AHF34780 | 0.059 | 0.384 | 0.154 | VH | CDRH3 | R99L | 1280 | 1736 |
| AHF34784 | 0.044 | 0.384 | 0.114 | VH | CDRH3 | R99M | 1280 | 1737 |
| AHF34782 | 0.056 | 0.384 | 0.147 | VH | CDRH3 | R99N | 1280 | 1738 |
| AHF34804 | 0.059 | 0.384 | 0.153 | VH | CDRH3 | R99P | 1280 | 1739 |
| AHF34815 | 0.046 | 0.384 | 0.119 | VH | CDRH3 | R99Q | 1280 | 1740 |
| AHF34789 | 0.054 | 0.384 | 0.140 | VH | CDRH3 | R99S | 1280 | 1741 |
| AHF34788 | 0.047 | 0.384 | 0.122 | VH | CDRH3 | R99T | 1280 | 1742 |
| AHF34777 | 0.050 | 0.384 | 0.131 | VH | CDRH3 | R99V | 1280 | 1743 |
| AHF34792 | 0.042 | 0.384 | 0.109 | VH | CDRH3 | R99W | 1280 | 1744 |
| AHF34796 | 0.051 | 0.384 | 0.133 | VH | CDRH3 | R99Y | 1280 | 1745 |
| AHF34849 | 0.120 | 0.465 | 0.257 | VH | CDRH3 | G100D | 1280 | 1746 |
| AHF34841 | 0.099 | 0.465 | 0.213 | VH | CDRH3 | G100E | 1280 | 1747 |
| AHF34845 | 0.171 | 0.465 | 0.368 | VH | CDRH3 | G100F | 1280 | 1748 |
| AHF34833 | 0.321 | 0.465 | 0.691 | VH | CDRH3 | G100G | 1280 | 1278 |
| AHF34837 | 0.297 | 0.465 | 0.640 | VH | CDRH3 | G100H | 1280 | 1749 |
| AHF34832 | 0.076 | 0.465 | 0.163 | VH | CDRH3 | G100I | 1280 | 1750 |
| AHF34826 | 0.237 | 0.465 | 0.511 | VH | CDRH3 | G100K | 1280 | 1751 |
| AHF34846 | 0.164 | 0.465 | 0.353 | VH | CDRH3 | G100L | 1280 | 1752 |
| AHF34859 | 0.225 | 0.465 | 0.484 | VH | CDRH3 | G100M | 1280 | 1753 |
| AHF34821 | 0.300 | 0.465 | 0.645 | VH | CDRH3 | G100N | 1280 | 1754 |
| AHF34847 | 0.049 | 0.465 | 0.104 | VH | CDRH3 | G100P | 1280 | 1755 |
| AHF34843 | 0.195 | 0.465 | 0.420 | VH | CDRH3 | G100R | 1280 | 1756 |
| AHF34857 | 0.266 | 0.465 | 0.573 | VH | CDRH3 | G100S | 1280 | 1757 |
| AHF34842 | 0.088 | 0.465 | 0.189 | VH | CDRH3 | G100T | 1280 | 1758 |
| AHF34834 | 0.070 | 0.465 | 0.150 | VH | CDRH3 | G100V | 1280 | 1759 |
| AHF34818 | 0.076 | 0.465 | 0.163 | VH | CDRH3 | G100W | 1280 | 1760 |
| AHF34850 | 0.229 | 0.465 | 0.492 | VH | CDRH3 | G100Y | 1280 | 1761 |
| AHF34904 | 0.569 | 0.465 | 1.224 | VH | CDRH3 | E101A | 1280 | 1448 |
| AHF34871 | 0.408 | 0.465 | 0.879 | VH | CDRH3 | E101D | 1280 | 1762 |
| AHF34907 | 0.174 | 0.465 | 0.375 | VH | CDRH3 | E101E | 1280 | 1278 |
| AHF34902 | 0.538 | 0.465 | 1.158 | VH | CDRH3 | E101G | 1280 | 1763 |
| AHF34898 | 0.580 | 0.465 | 1.249 | VH | CDRH3 | E101H | 1280 | 1447 |
| AHF34893 | 0.517 | 0.465 | 1.113 | VH | CDRH3 | E101I | 1280 | 1764 |
| AHF34903 | 0.531 | 0.465 | 1.142 | VH | CDRH3 | E101K | 1280 | 1765 |
| AHF34897 | 0.574 | 0.465 | 1.236 | VH | CDRH3 | E101L | 1280 | 1446 |
| AHF34906 | 0.549 | 0.465 | 1.182 | VH | CDRH3 | E101M | 1280 | 1766 |
| AHF34886 | 0.537 | 0.465 | 1.155 | VH | CDRH3 | E101N | 1280 | 1767 |
| AHF34892 | 0.483 | 0.465 | 1.040 | VH | CDRH3 | E101P | 1280 | 1768 |
| AHF34874 | 0.541 | 0.465 | 1.165 | VH | CDRH3 | E101R | 1280 | 1769 |
| AHF34873 | 0.540 | 0.465 | 1.163 | VH | CDRH3 | E101S | 1280 | 1770 |
| AHF34881 | 0.562 | 0.465 | 1.209 | VH | CDRH3 | E101T | 1280 | 1445 |
| AHF34869 | 0.443 | 0.465 | 0.953 | VH | CDRH3 | E101V | 1280 | 1771 |
| AHF34882 | 0.533 | 0.465 | 1.146 | VH | CDRH3 | E101W | 1280 | 1772 |
| AHF34896 | 0.534 | 0.465 | 1.149 | VH | CDRH3 | E101Y | 1280 | 1773 |
| AHF34949 | 0.186 | 0.460 | 0.405 | VH | CDRH3 | G102A | 1280 | 1774 |
| AHF34911 | 0.046 | 0.460 | 0.099 | VH | CDRH3 | G102D | 1280 | 1775 |
| AHF34923 | 0.090 | 0.460 | 0.196 | VH | CDRH3 | G102E | 1280 | 1776 |
| AHF34931 | 0.153 | 0.460 | 0.332 | VH | CDRH3 | G102F | 1280 | 1777 |
| AHF34933 | 0.254 | 0.460 | 0.552 | VH | CDRH3 | G102H | 1280 | 1778 |
| AHF34930 | 0.077 | 0.460 | 0.167 | VH | CDRH3 | G102I | 1280 | 1779 |
| AHF34914 | 0.206 | 0.460 | 0.449 | VH | CDRH3 | G102K | 1280 | 1780 |
| AHF34942 | 0.084 | 0.460 | 0.182 | VH | CDRH3 | G102L | 1280 | 1781 |
| AHF34910 | 0.111 | 0.460 | 0.241 | VH | CDRH3 | G102M | 1280 | 1782 |
| AHF34913 | 0.148 | 0.460 | 0.322 | VH | CDRH3 | G102N | 1280 | 1783 |
| AHF34932 | 0.080 | 0.460 | 0.175 | VH | CDRH3 | G102P | 1280 | 1784 |
| AHF34945 | 0.191 | 0.460 | 0.414 | VH | CDRH3 | G102R | 1280 | 1785 |
| AHF34955 | 0.111 | 0.460 | 0.241 | VH | CDRH3 | G102S | 1280 | 1786 |
| AHF34935 | 0.106 | 0.460 | 0.230 | VH | CDRH3 | G102T | 1280 | 1787 |
| AHF34948 | 0.121 | 0.460 | 0.264 | VH | CDRH3 | G102V | 1280 | 1788 |
| AHF34917 | 0.265 | 0.460 | 0.575 | VH | CDRH3 | G102W | 1280 | 1789 |
| AHF34915 | 0.217 | 0.460 | 0.472 | VH | CDRH3 | G102Y | 1280 | 1790 |
| AHF34993 | 0.048 | 0.460 | 0.104 | VH | CDRH3 | A103D | 1280 | 1791 |
| AHF34963 | 0.045 | 0.460 | 0.098 | VH | CDRH3 | A103E | 1280 | 1792 |
| AHF34965 | 0.093 | 0.460 | 0.202 | VH | CDRH3 | A103F | 1280 | 1793 |
| AHF34991 | 0.178 | 0.460 | 0.387 | VH | CDRH3 | A103G | 1280 | 1794 |
| AHF34981 | 0.050 | 0.460 | 0.108 | VH | CDRH3 | A103H | 1280 | 1795 |
| AHF34988 | 0.065 | 0.460 | 0.141 | VH | CDRH3 | A103I | 1280 | 1796 |
| AHF34957 | 0.255 | 0.460 | 0.555 | VH | CDRH3 | A103K | 1280 | 1797 |
| AHF34964 | 0.052 | 0.460 | 0.112 | VH | CDRH3 | A103L | 1280 | 1798 |
| AHF34977 | 0.048 | 0.460 | 0.105 | VH | CDRH3 | A103M | 1280 | 1799 |
| AHF34961 | 0.067 | 0.460 | 0.145 | VH | CDRH3 | A103N | 1280 | 1800 |
| AHF34958 | 0.134 | 0.460 | 0.292 | VH | CDRH3 | A103P | 1280 | 1801 |
| AHF34999 | 0.054 | 0.460 | 0.118 | VH | CDRH3 | A103Q | 1280 | 1802 |
| AHF35000 | 0.102 | 0.460 | 0.222 | VH | CDRH3 | A103R | 1280 | 1803 |
| AHF34960 | 0.256 | 0.460 | 0.556 | VH | CDRH3 | A103S | 1280 | 1804 |
| AHF34987 | 0.161 | 0.460 | 0.350 | VH | CDRH3 | A103T | 1280 | 1805 |
| AHF34966 | 0.154 | 0.460 | 0.334 | VH | CDRH3 | A103V | 1280 | 1806 |
| AHF34997 | 0.073 | 0.460 | 0.158 | VH | CDRH3 | A103W | 1280 | 1807 |
| AHF34994 | 0.187 | 0.460 | 0.406 | VH | CDRH3 | A103Y | 1280 | 1808 |
| AHF35041 | 0.242 | 0.470 | 0.514 | VH | CDRH3 | M104A | 1280 | 1809 |
| AHF35043 | 0.038 | 0.470 | 0.082 | VH | CDRH3 | M104D | 1280 | 1810 |
| AHF35007 | 0.198 | 0.470 | 0.422 | VH | CDRH3 | M104E | 1280 | 1811 |
| AHF35005 | 0.210 | 0.470 | 0.447 | VH | CDRH3 | M104F | 1280 | 1812 |
| AHF35040 | 0.203 | 0.470 | 0.431 | VH | CDRH3 | M104G | 1280 | 1813 |
| AHF35044 | 0.101 | 0.470 | 0.215 | VH | CDRH3 | M104H | 1280 | 1814 |
| AHF35025 | 0.410 | 0.470 | 0.874 | VH | CDRH3 | M104L | 1280 | 1815 |
| AHF35016 | 0.184 | 0.470 | 0.392 | VH | CDRH3 | M104N | 1280 | 1816 |
| AHF35009 | 0.227 | 0.470 | 0.482 | VH | CDRH3 | M104P | 1280 | 1817 |
| AHF35008 | 0.100 | 0.470 | 0.213 | VH | CDRH3 | M104Q | 1280 | 1818 |
| AHF35048 | 0.120 | 0.470 | 0.256 | VH | CDRH3 | M104R | 1280 | 1819 |
| AHF35014 | 0.144 | 0.470 | 0.307 | VH | CDRH3 | M104S | 1280 | 1820 |
| AHF35002 | 0.150 | 0.470 | 0.320 | VH | CDRH3 | M104T | 1280 | 1821 |
| AHF35046 | 0.062 | 0.470 | 0.131 | VH | CDRH3 | M104W | 1280 | 1822 |
| AHF35004 | 0.042 | 0.470 | 0.089 | VH | CDRH3 | M104Y | 1280 | 1823 |
| AHF35083 | 0.102 | 0.470 | 0.217 | VH | CDRH3 | D105E | 1280 | 1824 |
| AHF35075 | 0.062 | 0.470 | 0.131 | VH | CDRH3 | D105F | 1280 | 1825 |
| AHF35052 | 0.077 | 0.470 | 0.165 | VH | CDRH3 | D105G | 1280 | 1826 |
| AHF35091 | 0.329 | 0.470 | 0.700 | VH | CDRH3 | D105H | 1280 | 1827 |
| AHF35056 | 0.349 | 0.470 | 0.743 | VH | CDRH3 | D105I | 1280 | 1828 |
| AHF35090 | 0.235 | 0.470 | 0.501 | VH | CDRH3 | D105K | 1280 | 1829 |
| AHF35050 | 0.199 | 0.470 | 0.425 | VH | CDRH3 | D105L | 1280 | 1830 |
| AHF35057 | 0.093 | 0.470 | 0.198 | VH | CDRH3 | D105M | 1280 | 1831 |
| AHF35088 | 0.073 | 0.470 | 0.155 | VH | CDRH3 | D105P | 1280 | 1832 |
| AHF35082 | 0.322 | 0.470 | 0.686 | VH | CDRH3 | D105Q | 1280 | 1833 |
| AHF35065 | 0.194 | 0.470 | 0.414 | VH | CDRH3 | D105R | 1280 | 1834 |
| AHF35078 | 0.168 | 0.470 | 0.358 | VH | CDRH3 | D105S | 1280 | 1835 |
| AHF35080 | 0.209 | 0.470 | 0.444 | VH | CDRH3 | D105T | 1280 | 1836 |
| AHF35049 | 0.052 | 0.470 | 0.110 | VH | CDRH3 | D105V | 1280 | 1837 |
| AHF35079 | 0.203 | 0.470 | 0.432 | VH | CDRH3 | D105W | 1280 | 1838 |
| AHF35089 | 0.272 | 0.470 | 0.579 | VH | CDRH3 | D105Y | 1280 | 1839 |
| AHF35102 | 0.526 | 0.445 | 1.182 | VH | CDRH3 | Y106A | 1280 | 1450 |
| AHF35121 | 0.048 | 0.445 | 0.107 | VH | CDRH3 | Y106D | 1280 | 1840 |
| AHF35115 | 0.336 | 0.445 | 0.757 | VH | CDRH3 | Y106F | 1280 | 1841 |
| AHF35101 | 0.326 | 0.445 | 0.733 | VH | CDRH3 | Y106G | 1280 | 1842 |
| AHF35133 | 0.367 | 0.445 | 0.825 | VH | CDRH3 | Y106H | 1280 | 1843 |
| AHF35120 | 0.360 | 0.445 | 0.809 | VH | CDRH3 | Y106L | 1280 | 1844 |
| AHF35098 | 0.467 | 0.445 | 1.051 | VH | CDRH3 | Y106M | 1280 | 1845 |
| AHF35097 | 0.569 | 0.445 | 1.281 | VH | CDRH3 | Y106N | 1280 | 1449 |
| AHF35123 | 0.458 | 0.445 | 1.030 | VH | CDRH3 | Y106P | 1280 | 1846 |
| AHF35127 | 0.424 | 0.445 | 0.953 | VH | CDRH3 | Y106Q | 1280 | 1847 |
| AHF35116 | 0.399 | 0.445 | 0.897 | VH | CDRH3 | Y106R | 1280 | 1848 |
| AHF35137 | 0.394 | 0.445 | 0.887 | VH | CDRH3 | Y106S | 1280 | 1849 |
| AHF35110 | 0.449 | 0.445 | 1.010 | VH | CDRH3 | Y106T | 1280 | 1850 |
| AHF35094 | 0.507 | 0.445 | 1.141 | VH | CDRH3 | Y106V | 1280 | 1851 |
| AHF35107 | 0.521 | 0.445 | 1.171 | VH | CDRH3 | Y106W | 1280 | 1451 |

TABLE 27
Light Chain Mutant Fab Binding to Human TCRβV12-3 by ELISA

| | Human TCRβV12-3 binding | | | Mutations | | | SEQ | |
|---|---|---|---|---|---|---|---|---|
| | ELISA | | | in VL CDRs | | | ID NO | |
| Sequence | Mutation | WT | Ratio = | | | Point | Fab | Fab |
| ID | OD450nm | OD450nm | OD(Mutation)/OD(WT) | Chain | CDR | mutation | LC | HC |
| AHF35182 | 0.369 | 0.445 | 0.831 | VL | CDRL1 | S26A | 1852 | 1278 |
| AHF35175 | 0.246 | 0.445 | 0.553 | VL | CDRL1 | S26D | 1853 | 1278 |
| AHF35171 | 0.376 | 0.445 | 0.846 | VL | CDRL1 | S26F | 1854 | 1278 |
| AHF35167 | 0.294 | 0.445 | 0.660 | VL | CDRL1 | S26G | 1855 | 1278 |
| AHF35179 | 0.425 | 0.445 | 0.956 | VL | CDRL1 | S26H | 1856 | 1278 |
| AHF35141 | 0.395 | 0.445 | 0.888 | VL | CDRL1 | S26I | 1857 | 1278 |
| AHF35153 | 0.211 | 0.445 | 0.474 | VL | CDRL1 | S26K | 1858 | 1278 |
| AHF35183 | 0.448 | 0.445 | 1.008 | VL | CDRL1 | S26L | 1859 | 1278 |
| AHF35164 | 0.535 | 0.445 | 1.203 | VL | CDRL1 | S26M | 1429 | 1278 |
| AHF35157 | 0.353 | 0.445 | 0.794 | VL | CDRL1 | S26N | 1860 | 1278 |
| AHF35166 | 0.346 | 0.445 | 0.778 | VL | CDRL1 | S26P | 1861 | 1278 |
| AHF35168 | 0.440 | 0.445 | 0.989 | VL | CDRL1 | S26Q | 1862 | 1278 |
| AHF35184 | 0.437 | 0.445 | 0.983 | VL | CDRL1 | S26R | 1863 | 1278 |
| AHF35169 | 0.379 | 0.445 | 0.853 | VL | CDRL1 | S26T | 1864 | 1278 |
| AHF35163 | 0.450 | 0.445 | 1.012 | VL | CDRL1 | S26V | 1865 | 1278 |
| AHF35160 | 0.360 | 0.445 | 0.809 | VL | CDRL1 | S26W | 1866 | 1278 |
| AHF35158 | 0.421 | 0.445 | 0.947 | VL | CDRL1 | S26Y | 1867 | 1278 |
| AHF35217 | 0.369 | 0.460 | 0.802 | VL | CDRL1 | S27A | 1868 | 1278 |
| AHF35213 | 0.238 | 0.460 | 0.517 | VL | CDRL1 | S27D | 1869 | 1278 |
| AHF35209 | 0.265 | 0.460 | 0.577 | VL | CDRL1 | S27E | 1870 | 1278 |
| AHF35216 | 0.376 | 0.460 | 0.818 | VL | CDRL1 | S27F | 1871 | 1278 |
| AHF35200 | 0.397 | 0.460 | 0.863 | VL | CDRL1 | S27G | 1872 | 1278 |
| AHF35221 | 0.522 | 0.460 | 1.134 | VL | CDRL1 | S27H | 1873 | 1278 |
| AHF35192 | 0.430 | 0.460 | 0.935 | VL | CDRL1 | S27I | 1874 | 1278 |
| AHF35188 | 0.560 | 0.460 | 1.218 | VL | CDRL1 | S27K | 1287 | 1278 |
| AHF35198 | 0.383 | 0.460 | 0.833 | VL | CDRL1 | S27L | 1875 | 1278 |
| AHF35186 | 0.387 | 0.460 | 0.842 | VL | CDRL1 | S27M | 1876 | 1278 |
| AHF35222 | 0.432 | 0.460 | 0.939 | VL | CDRL1 | S27N | 1877 | 1278 |
| AHF35226 | 0.334 | 0.460 | 0.726 | VL | CDRL1 | S27P | 1878 | 1278 |
| AHF35199 | 0.425 | 0.460 | 0.923 | VL | CDRL1 | S27Q | 1879 | 1278 |
| AHF35230 | 0.546 | 0.460 | 1.188 | VL | CDRL1 | S27R | 1430 | 1278 |
| AHF35219 | 0.414 | 0.460 | 0.900 | VL | CDRL1 | S27T | 1880 | 1278 |
| AHF35208 | 0.369 | 0.460 | 0.803 | VL | CDRL1 | S27V | 1881 | 1278 |
| AHF35223 | 0.357 | 0.460 | 0.777 | VL | CDRL1 | S27W | 1882 | 1278 |
| AHF35231 | 0.456 | 0.460 | 0.992 | VL | CDRL1 | S27Y | 1883 | 1278 |
| AHF35248 | 0.444 | 0.460 | 0.965 | VL | CDRL1 | S28A | 1884 | 1278 |
| AHF35276 | 0.391 | 0.460 | 0.851 | VL | CDRL1 | S28D | 1885 | 1278 |
| AHF35267 | 0.385 | 0.460 | 0.838 | VL | CDRL1 | S28E | 1886 | 1278 |
| AHF35247 | 0.387 | 0.460 | 0.841 | VL | CDRL1 | S28F | 1887 | 1278 |
| AHF35268 | 0.362 | 0.460 | 0.788 | VL | CDRL1 | S28G | 1888 | 1278 |
| AHF35233 | 0.415 | 0.460 | 0.902 | VL | CDRL1 | S28H | 1889 | 1278 |
| AHF35235 | 0.432 | 0.460 | 0.939 | VL | CDRL1 | S28I | 1890 | 1278 |
| AHF35238 | 0.548 | 0.460 | 1.191 | VL | CDRL1 | S28K | 1452 | 1278 |
| AHF35243 | 0.449 | 0.460 | 0.976 | VL | CDRL1 | S28M | 1891 | 1278 |
| AHF35256 | 0.498 | 0.460 | 1.083 | VL | CDRL1 | S28N | 1892 | 1278 |
| AHF35274 | 0.484 | 0.460 | 1.052 | VL | CDRL1 | S28P | 1893 | 1278 |
| AHF35239 | 0.407 | 0.460 | 0.885 | VL | CDRL1 | S28Q | 1894 | 1278 |
| AHF35269 | 0.495 | 0.460 | 1.076 | VL | CDRL1 | S28R | 1895 | 1278 |
| AHF35260 | 0.379 | 0.460 | 0.824 | VL | CDRL1 | S28T | 1896 | 1278 |
| AHF35273 | 0.412 | 0.460 | 0.895 | VL | CDRL1 | S28V | 1897 | 1278 |
| AHF35266 | 0.407 | 0.460 | 0.884 | VL | CDRL1 | S28W | 1898 | 1278 |
| AHF35261 | 0.444 | 0.460 | 0.966 | VL | CDRL1 | S28Y | 1899 | 1278 |
| AHF35319 | 0.301 | 0.508 | 0.592 | VL | CDRL1 | V29A | 1900 | 1278 |
| AHF35278 | 0.051 | 0.508 | 0.101 | VL | CDRL1 | V29D | 1901 | 1278 |
| AHF35301 | 0.055 | 0.508 | 0.109 | VL | CDRL1 | V29E | 1902 | 1278 |
| AHF35300 | 0.090 | 0.508 | 0.178 | VL | CDRL1 | V29F | 1903 | 1278 |
| AHF35313 | 0.208 | 0.508 | 0.410 | VL | CDRL1 | V29G | 1904 | 1278 |
| AHF35292 | 0.072 | 0.508 | 0.141 | VL | CDRL1 | V29H | 1905 | 1278 |
| AHF35308 | 0.457 | 0.508 | 0.900 | VL | CDRL1 | V29I | 1906 | 1278 |
| AHF35295 | 0.332 | 0.508 | 0.653 | VL | CDRL1 | V29L | 1907 | 1278 |
| AHF35283 | 0.217 | 0.508 | 0.427 | VL | CDRL1 | V29M | 1908 | 1278 |
| AHF35282 | 0.059 | 0.508 | 0.116 | VL | CDRL1 | V29N | 1909 | 1278 |
| AHF35284 | 0.167 | 0.508 | 0.328 | VL | CDRL1 | V29P | 1910 | 1278 |
| AHF35277 | 0.151 | 0.508 | 0.297 | VL | CDRL1 | V29Q | 1911 | 1278 |
| AHF35324 | 0.298 | 0.508 | 0.587 | VL | CDRL1 | V29R | 1912 | 1278 |
| AHF35294 | 0.412 | 0.508 | 0.810 | VL | CDRL1 | V29T | 1913 | 1278 |
| AHF35299 | 0.064 | 0.508 | 0.126 | VL | CDRL1 | V29W | 1914 | 1278 |
| AHF35303 | 0.113 | 0.508 | 0.222 | VL | CDRL1 | V29Y | 1915 | 1278 |
| AHF35325 | 0.301 | 0.508 | 0.592 | VL | CDRL1 | N30A | 1916 | 1278 |
| AHF35360 | 0.069 | 0.508 | 0.136 | VL | CDRL1 | N30D | 1917 | 1278 |
| AHF35363 | 0.211 | 0.508 | 0.415 | VL | CDRL1 | N30E | 1918 | 1278 |
| AHF35354 | 0.070 | 0.508 | 0.138 | VL | CDRL1 | N30F | 1919 | 1278 |
| AHF35355 | 0.116 | 0.508 | 0.228 | VL | CDRL1 | N30G | 1920 | 1278 |
| AHF35362 | 0.135 | 0.508 | 0.266 | VL | CDRL1 | N30H | 1921 | 1278 |
| AHF35352 | 0.367 | 0.508 | 0.723 | VL | CDRL1 | N30K | 1922 | 1278 |
| AHF35328 | 0.238 | 0.508 | 0.469 | VL | CDRL1 | N30L | 1923 | 1278 |
| AHF35350 | 0.333 | 0.508 | 0.655 | VL | CDRL1 | N30M | 1924 | 1278 |
| AHF35365 | 0.071 | 0.508 | 0.139 | VL | CDRL1 | N30P | 1925 | 1278 |
| AHF35341 | 0.183 | 0.508 | 0.360 | VL | CDRL1 | N30Q | 1926 | 1278 |
| AHF35340 | 0.416 | 0.508 | 0.819 | VL | CDRL1 | N30R | 1927 | 1278 |
| AHF35368 | 0.167 | 0.508 | 0.329 | VL | CDRL1 | N30S | 1928 | 1278 |
| AHF35366 | 0.241 | 0.508 | 0.474 | VL | CDRL1 | N30T | 1929 | 1278 |
| AHF35364 | 0.521 | 0.508 | 1.025 | VL | CDRL1 | N30V | 1930 | 1278 |
| AHF35326 | 0.236 | 0.508 | 0.464 | VL | CDRL1 | N30W | 1931 | 1278 |
| AHF35329 | 0.186 | 0.508 | 0.365 | VL | CDRL1 | N30Y | 1932 | 1278 |
| AHF35402 | 0.209 | 0.513 | 0.408 | VL | CDRL1 | Y31A | 1933 | 1278 |
| AHF35377 | 0.053 | 0.513 | 0.102 | VL | CDRL1 | Y31D | 1934 | 1278 |
| AHF35380 | 0.068 | 0.513 | 0.133 | VL | CDRL1 | Y31E | 1935 | 1278 |
| AHF35384 | 0.563 | 0.513 | 1.096 | VL | CDRL1 | Y31F | 1936 | 1278 |
| AHF35375 | 0.108 | 0.513 | 0.210 | VL | CDRL1 | Y31G | 1937 | 1278 |
| AHF35405 | 0.242 | 0.513 | 0.472 | VL | CDRL1 | Y31H | 1938 | 1278 |
| AHF35395 | 0.066 | 0.513 | 0.128 | VL | CDRL1 | Y31I | 1939 | 1278 |
| AHF35408 | 0.316 | 0.513 | 0.615 | VL | CDRL1 | Y31K | 1940 | 1278 |
| AHF35416 | 0.086 | 0.513 | 0.168 | VL | CDRL1 | Y31L | 1941 | 1278 |
| AHF35372 | 0.177 | 0.513 | 0.345 | VL | CDRL1 | Y31M | 1942 | 1278 |
| AHF35406 | 0.195 | 0.513 | 0.379 | VL | CDRL1 | Y31N | 1943 | 1278 |
| AHF35378 | 0.115 | 0.513 | 0.224 | VL | CDRL1 | Y31P | 1944 | 1278 |
| AHF35404 | 0.146 | 0.513 | 0.284 | VL | CDRL1 | Y31Q | 1945 | 1278 |
| AHF35415 | 0.282 | 0.513 | 0.549 | VL | CDRL1 | Y31R | 1946 | 1278 |
| AHF35413 | 0.074 | 0.513 | 0.144 | VL | CDRL1 | Y31T | 1947 | 1278 |
| AHF35379 | 0.139 | 0.513 | 0.270 | VL | CDRL1 | Y31V | 1948 | 1278 |
| AHF35370 | 0.326 | 0.513 | 0.635 | VL | CDRL1 | Y31Y | 1280 | 1278 |
| AHF35445 | 0.611 | 0.513 | 1.190 | VL | CDRL1 | I32A | 1291 | 1278 |
| AHF35428 | 0.285 | 0.513 | 0.556 | VL | CDRL1 | I32D | 1949 | 1278 |
| AHF35425 | 0.111 | 0.513 | 0.217 | VL | CDRL1 | I32F | 1950 | 1278 |
| AHF35424 | 0.138 | 0.513 | 0.269 | VL | CDRL1 | I32H | 1951 | 1278 |
| AHF35459 | 0.227 | 0.513 | 0.442 | VL | CDRL1 | I32I | 1280 | 1278 |
| AHF35448 | 0.090 | 0.513 | 0.174 | VL | CDRL1 | I32K | 1952 | 1278 |
| AHF35450 | 0.467 | 0.513 | 0.910 | VL | CDRL1 | I32L | 1953 | 1278 |
| AHF35433 | 0.496 | 0.513 | 0.966 | VL | CDRL1 | I32M | 1954 | 1278 |
| AHF35418 | 0.102 | 0.513 | 0.198 | VL | CDRL1 | I32N | 1955 | 1278 |
| AHF35439 | 0.157 | 0.513 | 0.307 | VL | CDRL1 | I32P | 1956 | 1278 |
| AHF35432 | 0.310 | 0.513 | 0.605 | VL | CDRL1 | I32Q | 1957 | 1278 |
| AHF35423 | 0.048 | 0.513 | 0.093 | VL | CDRL1 | I32R | 1958 | 1278 |
| AHF35455 | 0.578 | 0.513 | 1.126 | VL | CDRL1 | I32S | 1959 | 1278 |
| AHF35431 | 0.374 | 0.513 | 0.728 | VL | CDRL1 | I32T | 1960 | 1278 |
| AHF35447 | 0.086 | 0.513 | 0.167 | VL | CDRL1 | I32W | 1961 | 1278 |
| AHF35458 | 0.079 | 0.513 | 0.153 | VL | CDRL1 | I32Y | 1962 | 1278 |
| AHF35476 | 0.061 | 0.527 | 0.116 | VL | CDRL1 | Y33A | 1963 | 1278 |
| AHF35493 | 0.309 | 0.527 | 0.585 | VL | CDRL1 | Y33D | 1964 | 1278 |
| AHF35461 | 0.132 | 0.527 | 0.249 | VL | CDRL1 | Y33F | 1965 | 1278 |
| AHF35469 | 0.069 | 0.527 | 0.131 | VL | CDRL1 | Y33G | 1966 | 1278 |
| AHF35486 | 0.121 | 0.527 | 0.230 | VL | CDRL1 | Y33H | 1967 | 1278 |
| AHF35475 | 0.042 | 0.527 | 0.080 | VL | CDRL1 | Y33I | 1968 | 1278 |
| AHF35491 | 0.199 | 0.527 | 0.377 | VL | CDRL1 | Y33L | 1969 | 1278 |
| AHF35507 | 0.049 | 0.527 | 0.094 | VL | CDRL1 | Y33N | 1970 | 1278 |
| AHF35504 | 0.066 | 0.527 | 0.126 | VL | CDRL1 | Y33P | 1971 | 1278 |
| AHF35501 | 0.086 | 0.527 | 0.162 | VL | CDRL1 | Y33R | 1972 | 1278 |
| AHF35506 | 0.200 | 0.527 | 0.379 | VL | CDRL1 | Y33S | 1973 | 1278 |
| AHF35502 | 0.105 | 0.527 | 0.199 | VL | CDRL1 | Y33T | 1974 | 1278 |
| AHF35489 | 0.046 | 0.527 | 0.087 | VL | CDRL1 | Y33V | 1975 | 1278 |
| AHF35462 | 0.282 | 0.527 | 0.534 | VL | CDRL1 | Y33W | 1976 | 1278 |
| AHF35512 | 0.233 | 0.527 | 0.442 | VL | CDRL2 | Y49A | 1977 | 1278 |
| AHF35531 | 0.053 | 0.527 | 0.101 | VL | CDRL2 | Y49D | 1978 | 1278 |
| AHF35517 | 0.100 | 0.527 | 0.189 | VL | CDRL2 | Y49E | 1979 | 1278 |
| AHF35539 | 0.477 | 0.527 | 0.904 | VL | CDRL2 | Y49F | 1980 | 1278 |
| AHF35520 | 0.188 | 0.527 | 0.357 | VL | CDRL2 | Y49G | 1981 | 1278 |
| AHF35522 | 0.351 | 0.527 | 0.665 | VL | CDRL2 | Y49H | 1982 | 1278 |
| AHF35537 | 0.472 | 0.527 | 0.894 | VL | CDRL2 | Y49I | 1983 | 1278 |
| AHF35533 | 0.590 | 0.527 | 1.118 | VL | CDRL2 | Y49K | 1984 | 1278 |
| AHF35527 | 0.313 | 0.527 | 0.593 | VL | CDRL2 | Y49L | 1985 | 1278 |
| AHF35528 | 0.330 | 0.527 | 0.626 | VL | CDRL2 | Y49M | 1986 | 1278 |
| AHF35535 | 0.129 | 0.527 | 0.244 | VL | CDRL2 | Y49N | 1987 | 1278 |
| AHF35509 | 0.162 | 0.527 | 0.307 | VL | CDRL2 | Y49P | 1988 | 1278 |
| AHF35513 | 0.303 | 0.527 | 0.575 | VL | CDRL2 | Y49Q | 1989 | 1278 |
| AHF35541 | 0.410 | 0.527 | 0.778 | VL | CDRL2 | Y49T | 1990 | 1278 |
| AHF35511 | 0.139 | 0.527 | 0.263 | VL | CDRL2 | Y49V | 1991 | 1278 |
| AHF35518 | 0.330 | 0.527 | 0.626 | VL | CDRL2 | Y49W | 1992 | 1278 |
| AHF35534 | 0.483 | 0.527 | 0.915 | VL | CDRL2 | Y49Y | 1280 | 1278 |
| AHF35557 | 0.206 | 0.359 | 0.574 | VL | CDRL2 | T50A | 1993 | 1278 |
| AHF35571 | 0.059 | 0.359 | 0.164 | VL | CDRL2 | T50D | 1994 | 1278 |
| AHF35581 | 0.055 | 0.359 | 0.154 | VL | CDRL2 | T50E | 1995 | 1278 |
| AHF35573 | 0.103 | 0.359 | 0.286 | VL | CDRL2 | T50F | 1996 | 1278 |
| AHF35599 | 0.068 | 0.359 | 0.190 | VL | CDRL2 | T50G | 1997 | 1278 |
| AHF35563 | 0.039 | 0.359 | 0.108 | VL | CDRL2 | T50H | 1998 | 1278 |
| AHF35577 | 0.281 | 0.359 | 0.782 | VL | CDRL2 | T50I | 1999 | 1278 |
| AHF35596 | 0.060 | 0.359 | 0.168 | VL | CDRL2 | T50K | 2000 | 1278 |
| AHF35587 | 0.067 | 0.359 | 0.187 | VL | CDRL2 | T50L | 2001 | 1278 |
| AHF35567 | 0.089 | 0.359 | 0.247 | VL | CDRL2 | T50M | 2002 | 1278 |
| AHF35574 | 0.073 | 0.359 | 0.204 | VL | CDRL2 | T50N | 2003 | 1278 |
| AHF35595 | 0.395 | 0.359 | 1.099 | VL | CDRL2 | T50P | 2004 | 1278 |
| AHF35554 | 0.220 | 0.359 | 0.613 | VL | CDRL2 | T50Q | 2005 | 1278 |
| AHF35568 | 0.062 | 0.359 | 0.173 | VL | CDRL2 | T50R | 2006 | 1278 |
| AHF35553 | 0.313 | 0.359 | 0.871 | VL | CDRL2 | T50S | 2007 | 1278 |
| AHF35588 | 0.214 | 0.359 | 0.596 | VL | CDRL2 | T50T | 1280 | 1278 |
| AHF35584 | 0.115 | 0.359 | 0.321 | VL | CDRL2 | T50W | 2008 | 1278 |
| AHF35560 | 0.067 | 0.359 | 0.187 | VL | CDRL2 | T50Y | 2009 | 1278 |
| AHF35601 | 0.301 | 0.359 | 0.840 | VL | CDRL2 | S51A | 2010 | 1278 |
| AHF35614 | 0.237 | 0.359 | 0.659 | VL | CDRL2 | S51C | 2011 | 1278 |
| AHF35609 | 0.361 | 0.359 | 1.006 | VL | CDRL2 | S51D | 2012 | 1278 |
| AHF35637 | 0.213 | 0.359 | 0.592 | VL | CDRL2 | S51E | 2013 | 1278 |
| AHF35624 | 0.246 | 0.359 | 0.685 | VL | CDRL2 | S51F | 2014 | 1278 |
| AHF35603 | 0.205 | 0.359 | 0.570 | VL | CDRL2 | S51G | 2015 | 1278 |
| AHF35626 | 0.305 | 0.359 | 0.849 | VL | CDRL2 | S51H | 2016 | 1278 |
| AHF35630 | 0.269 | 0.359 | 0.748 | VL | CDRL2 | S51I | 2017 | 1278 |
| AHF35625 | 0.456 | 0.359 | 1.270 | VL | CDRL2 | S51K | 1454 | 1278 |
| AHF35635 | 0.359 | 0.359 | 1.001 | VI | CDRL2 | S51L | 2018 | 1278 |
| AHF35605 | 0.477 | 0.359 | 1.328 | VL | CDRL2 | S51N | 1453 | 1278 |
| AHF35608 | 0.365 | 0.359 | 1.017 | VL | CDRL2 | S51P | 2019 | 1278 |
| AHF35642 | 0.295 | 0.359 | 0.823 | VL | CDRL2 | S51Q | 2020 | 1278 |
| AHF35617 | 0.399 | 0.359 | 1.111 | VL | CDRL2 | S51R | 2021 | 1278 |
| AHF35641 | 0.327 | 0.359 | 0.912 | VL | CDRL2 | S51T | 2022 | 1278 |
| AHF35613 | 0.326 | 0.359 | 0.908 | VL | CDRL2 | S51V | 2023 | 1278 |
| AHF35631 | 0.275 | 0.359 | 0.766 | VL | CDRL2 | S51W | 2024 | 1278 |
| AHF35602 | 0.305 | 0.359 | 0.850 | VL | CDRL2 | S51Y | 2025 | 1278 |
| AHF35668 | 0.524 | 0.483 | 1.085 | VL | CDRL2 | N52A | 2026 | 1278 |
| AHF35659 | 0.326 | 0.483 | 0.676 | VL | CDRL2 | N52D | 2027 | 1278 |
| AHF35658 | 0.402 | 0.483 | 0.834 | VL | CDRL2 | N52E | 2028 | 1278 |
| AHF35680 | 0.455 | 0.483 | 0.942 | VL | CDRL2 | N52F | 2029 | 1278 |
| AHF35667 | 0.427 | 0.483 | 0.884 | VL | CDRL2 | N52G | 2030 | 1278 |
| AHF35681 | 0.343 | 0.483 | 0.710 | VL | CDRL2 | N52H | 2031 | 1278 |
| AHF35664 | 0.314 | 0.483 | 0.651 | VL | CDRL2 | N52I | 2032 | 1278 |
| AHF35675 | 0.674 | 0.483 | 1.396 | VL | CDRL2 | N52K | 1456 | 1278 |
| AHF35649 | 0.495 | 0.483 | 1.026 | VL | CDRL2 | N52L | 2033 | 1278 |
| AHF35673 | 0.449 | 0.483 | 0.931 | VL | CDRL2 | N52M | 2034 | 1278 |
| AHF35679 | 0.107 | 0.483 | 0.222 | VL | CDRL2 | N52P | 2035 | 1278 |
| AHF35650 | 0.701 | 0.483 | 1.452 | VL | CDRL2 | N52R | 1455 | 1278 |
| AHF35690 | 0.269 | 0.483 | 0.556 | VL | CDRL2 | N52S | 2036 | 1278 |
| AHF35645 | 0.476 | 0.483 | 0.987 | VL | CDRL2 | N52V | 2037 | 1278 |
| AHF35661 | 0.447 | 0.483 | 0.926 | VL | CDRL2 | N52W | 2038 | 1278 |
| AHF35684 | 0.381 | 0.483 | 0.790 | VL | CDRL2 | N52Y | 2039 | 1278 |
| AHF35718 | 0.421 | 0.483 | 0.873 | VL | CDRL2 | L53A | 2040 | 1278 |
| AHF35716 | 0.304 | 0.483 | 0.630 | VL | CDRL2 | L53C | 2041 | 1278 |
| AHF35699 | 0.059 | 0.483 | 0.121 | VL | CDRL2 | L53D | 2042 | 1278 |
| AHF35735 | 0.171 | 0.483 | 0.353 | VL | CDRL2 | L53E | 2043 | 1278 |
| AHF35707 | 0.360 | 0.483 | 0.745 | VL | CDRL2 | L53F | 2044 | 1278 |
| AHF35697 | 0.196 | 0.483 | 0.406 | VL | CDRL2 | L53G | 2045 | 1278 |
| AHF35709 | 0.514 | 0.483 | 1.064 | VL | CDRL2 | L53H | 2046 | 1278 |
| AHF35712 | 0.301 | 0.483 | 0.624 | VL | CDRL2 | L53I | 2047 | 1278 |
| AHF35693 | 0.462 | 0.483 | 0.957 | VL | CDRL2 | L53K | 2048 | 1278 |
| AHF35711 | 0.419 | 0.483 | 0.868 | VL | CDRL2 | L53N | 2049 | 1278 |
| AHF35731 | 0.357 | 0.483 | 0.740 | VL | CDRL2 | L53P | 2050 | 1278 |
| AHF35732 | 0.392 | 0.483 | 0.813 | VL | CDRL2 | L53R | 2051 | 1278 |
| AHF35725 | 0.053 | 0.483 | 0.110 | VL | CDRL2 | L53T | 2052 | 1278 |
| AHF35694 | 0.339 | 0.483 | 0.703 | VL | CDRL2 | L53V | 2053 | 1278 |
| AHF35708 | 0.291 | 0.483 | 0.603 | VL | CDRL2 | L53W | 2054 | 1278 |
| AHF35724 | 0.410 | 0.483 | 0.850 | VL | CDRL2 | L53Y | 2055 | 1278 |
| AHF35748 | 0.122 | 0.519 | 0.235 | VL | CDRL2 | A54D | 2056 | 1278 |
| AHF35769 | 0.232 | 0.519 | 0.448 | VL | CDRL2 | A54E | 2057 | 1278 |
| AHF35742 | 0.509 | 0.519 | 0.980 | VL | CDRL2 | A54F | 2058 | 1278 |
| AHF35777 | 0.388 | 0.519 | 0.748 | VL | CDRL2 | A54G | 2059 | 1278 |
| AHF35751 | 0.510 | 0.519 | 0.983 | VL | CDRL2 | A54H | 2060 | 1278 |
| AHF35776 | 0.512 | 0.519 | 0.985 | VL | CDRL2 | A54I | 2061 | 1278 |
| AHF35738 | 0.504 | 0.519 | 0.970 | VL | CDRL2 | A54K | 2062 | 1278 |
| AHF35757 | 0.397 | 0.519 | 0.765 | VL | CDRL2 | A54N | 2063 | 1278 |
| AHF35784 | 0.408 | 0.519 | 0.785 | VL | CDRL2 | A54P | 2064 | 1278 |
| AHF35783 | 0.400 | 0.519 | 0.771 | VL | CDRL2 | A54Q | 2065 | 1278 |
| AHF35760 | 0.669 | 0.519 | 1.289 | VL | CDRL2 | A54R | 1457 | 1278 |
| AHF35752 | 0.495 | 0.519 | 0.953 | VL | CDRL2 | A54T | 2066 | 1278 |
| AHF35743 | 0.589 | 0.519 | 1.135 | VL | CDRL2 | A54V | 2067 | 1278 |
| AHF35758 | 0.053 | 0.519 | 0.102 | VL | CDRL2 | A54Y | 2068 | 1278 |
| AHF35815 | 0.481 | 0.519 | 0.927 | VL | CDRL2 | P55D | 2069 | 1278 |
| AHF35801 | 0.511 | 0.519 | 0.984 | VL | CDRL2 | P55E | 2070 | 1278 |
| AHF35805 | 0.413 | 0.519 | 0.795 | VL | CDRL2 | P55F | 2071 | 1278 |
| AHF35825 | 0.476 | 0.519 | 0.916 | VL | CDRL2 | P55G | 2072 | 1278 |
| AHF35811 | 0.464 | 0.519 | 0.894 | VL | CDRL2 | P55H | 2073 | 1278 |
| AHF35790 | 0.470 | 0.519 | 0.906 | VL | CDRL2 | P55I | 2074 | 1278 |
| AHF35827 | 0.530 | 0.519 | 1.022 | VL | CDRL2 | P55L | 2075 | 1278 |
| AHF35803 | 0.448 | 0.519 | 0.863 | VL | CDRL2 | P55M | 2076 | 1278 |
| AHF35795 | 0.407 | 0.519 | 0.784 | VL | CDRL2 | P55N | 2077 | 1278 |
| AHF35821 | 0.536 | 0.519 | 1.032 | VL | CDRL2 | P55Q | 2078 | 1278 |
| AHF35792 | 0.833 | 0.519 | 1.605 | VL | CDRL2 | P55R | 1458 | 1278 |
| AHF35794 | 0.452 | 0.519 | 0.870 | VL | CDRL2 | P55S | 2079 | 1278 |
| AHF35826 | 0.553 | 0.519 | 1.066 | VL | CDRL2 | P55T | 2080 | 1278 |
| AHF35828 | 0.531 | 0.519 | 1.023 | VL | CDRL2 | P55V | 2081 | 1278 |
| AHF35817 | 0.515 | 0.519 | 0.991 | VL | CDRL2 | P55W | 2082 | 1278 |
| AHF35812 | 0.335 | 0.519 | 0.645 | VL | CDRL3 | P55Y | 2083 | 1278 |
| AHF35838 | 0.113 | 0.526 | 0.215 | VL | CDRL3 | Q88D | 2084 | 1278 |
| AHF35844 | 0.267 | 0.526 | 0.507 | VL | CDRL3 | Q88E | 2085 | 1278 |
| AHF35849 | 0.054 | 0.526 | 0.103 | VL | CDRL3 | Q88F | 2086 | 1278 |
| AHF35832 | 0.096 | 0.526 | 0.182 | VL | CDRL3 | Q88H | 2087 | 1278 |
| AHF35872 | 0.056 | 0.526 | 0.107 | VL | CDRL3 | Q88I | 2088 | 1278 |
| AHF35852 | 0.205 | 0.526 | 0.389 | VL | CDRL3 | Q88L | 2089 | 1278 |
| AHF35845 | 0.154 | 0.526 | 0.292 | VL | CDRL3 | Q88M | 2090 | 1278 |
| AHF35859 | 0.189 | 0.526 | 0.359 | VL | CDRL3 | Q88N | 2091 | 1278 |
| AHF35871 | 0.134 | 0.526 | 0.255 | VL | CDRL3 | Q88P | 2092 | 1278 |
| AHF35850 | 0.054 | 0.526 | 0.103 | VL | CDRL3 | Q88R | 2093 | 1278 |
| AHF35842 | 0.100 | 0.526 | 0.189 | VL | CDRL3 | Q88S | 2094 | 1278 |
| AHF35863 | 0.090 | 0.526 | 0.172 | VL | CDRL3 | Q88T | 2095 | 1278 |
| AHF35858 | 0.098 | 0.526 | 0.187 | VL | CDRL3 | Q88V | 2096 | 1278 |
| AHF35830 | 0.064 | 0.526 | 0.121 | VL | CDRL3 | Q88W | 2097 | 1278 |
| AHF35870 | 0.193 | 0.526 | 0.367 | VL | CDRL3 | Q88Y | 2098 | 1278 |
| AHF35908 | 0.111 | 0.526 | 0.212 | VL | CDRL3 | Q89A | 2099 | 1278 |
| AHF35897 | 0.055 | 0.526 | 0.104 | VL | CDRL3 | Q89D | 2100 | 1278 |
| AHF35892 | 0.303 | 0.526 | 0.576 | VL | CDRL3 | Q89E | 2101 | 1278 |
| AHF35910 | 0.070 | 0.526 | 0.133 | VL | CDRL3 | Q89F | 2102 | 1278 |
| AHF35887 | 0.163 | 0.526 | 0.310 | VL | CDRL3 | Q89G | 2103 | 1278 |
| AHF35880 | 0.323 | 0.526 | 0.615 | VL | CDRL3 | Q89H | 2104 | 1278 |
| AHF35912 | 0.090 | 0.526 | 0.170 | VL | CDRL3 | Q89I | 2105 | 1278 |
| AHF35918 | 0.245 | 0.526 | 0.466 | VL | CDRL3 | Q89K | 2106 | 1278 |
| AHF35914 | 0.105 | 0.526 | 0.199 | VL | CDRL3 | Q89L | 2107 | 1278 |
| AHF35905 | 0.070 | 0.526 | 0.133 | VL | CDRL3 | Q89M | 2108 | 1278 |
| AHF35882 | 0.050 | 0.526 | 0.095 | VL | CDRL3 | Q89P | 2109 | 1278 |
| AHF35898 | 0.184 | 0.526 | 0.349 | VL | CDRL3 | Q89R | 2110 | 1278 |
| AHF35902 | 0.123 | 0.526 | 0.233 | VL | CDRL3 | Q89S | 2111 | 1278 |
| AHF35909 | 0.177 | 0.526 | 0.337 | VL | CDRL3 | Q89T | 2112 | 1278 |
| AHF35884 | 0.195 | 0.526 | 0.372 | VL | CDRL3 | Q89V | 2113 | 1278 |
| AHF35889 | 0.058 | 0.526 | 0.110 | VL | CDRL3 | Q89Y | 2114 | 1278 |
| AHF35942 | 0.261 | 0.482 | 0.540 | VL | CDRL3 | F90A | 2115 | 1278 |
| AHF35939 | 0.052 | 0.482 | 0.108 | VL | CDRL3 | F90D | 2116 | 1278 |
| AHF35931 | 0.052 | 0.482 | 0.107 | VL | CDRL3 | F90E | 2117 | 1278 |
| AHF35938 | 0.098 | 0.482 | 0.202 | VL | CDRL3 | F90G | 2118 | 1278 |
| AHF35941 | 0.111 | 0.482 | 0.229 | VL | CDRL3 | F90H | 2119 | 1278 |
| AHF35968 | 0.132 | 0.482 | 0.274 | VL | CDRL3 | F90I | 2120 | 1278 |
| AHF35929 | 0.449 | 0.482 | 0.931 | VL | CDRL3 | F90K | 2121 | 1278 |
| AHF35926 | 0.480 | 0.482 | 0.995 | VL | CDRL3 | F90L | 2122 | 1278 |
| AHF35963 | 0.439 | 0.482 | 0.910 | VL | CDRL3 | F90M | 2123 | 1278 |
| AHF35949 | 0.302 | 0.482 | 0.626 | VL | CDRL3 | F90N | 2124 | 1278 |
| AHF35960 | 0.052 | 0.482 | 0.108 | VL | CDRL3 | F90P | 2125 | 1278 |
| AHF35937 | 0.326 | 0.482 | 0.675 | VL | CDRL3 | F90Q | 2126 | 1278 |
| AHF35923 | 0.229 | 0.482 | 0.474 | VL | CDRL3 | F90R | 2127 | 1278 |
| AHF35921 | 0.482 | 0.482 | 0.999 | VL | CDRL3 | F90S | 2128 | 1278 |
| AHF35957 | 0.112 | 0.482 | 0.232 | VL | CDRL3 | F90T | 2129 | 1278 |
| AHF35961 | 0.336 | 0.482 | 0.697 | VL | CDRL3 | F90V | 2130 | 1278 |
| AHF35922 | 0.063 | 0.482 | 0.130 | VL | CDRL3 | F90W | 2131 | 1278 |
| AHF35927 | 0.352 | 0.482 | 0.729 | VL | CDRL3 | F90Y | 2132 | 1278 |
| AHF35992 | 0.228 | 0.482 | 0.473 | VL | CDRL3 | T91A | 2133 | 1278 |
| AHF35988 | 0.042 | 0.482 | 0.087 | VL | CDRL3 | T91D | 2134 | 1278 |
| AHF35978 | 0.044 | 0.482 | 0.091 | VL | CDRL3 | T91E | 2135 | 1278 |
| AHF35979 | 0.129 | 0.482 | 0.268 | VL | CDRL3 | T91F | 2136 | 1278 |
| AHF35981 | 0.193 | 0.482 | 0.400 | VL | CDRL3 | T91G | 2137 | 1278 |
| AHF35985 | 0.286 | 0.482 | 0.592 | VL | CDRL3 | T91H | 2138 | 1278 |
| AHF36003 | 0.310 | 0.482 | 0.642 | VL | CDRL3 | T91I | 2139 | 1278 |
| AHF35971 | 0.203 | 0.482 | 0.420 | VL | CDRL3 | T91K | 2140 | 1278 |
| AHF36008 | 0.062 | 0.482 | 0.128 | VL | CDRL3 | T91L | 2141 | 1278 |
| AHF35970 | 0.143 | 0.482 | 0.296 | VL | CDRL3 | T91M | 2142 | 1278 |
| AHF35977 | 0.247 | 0.482 | 0.513 | VL | CDRL3 | T91N | 2143 | 1278 |
| AHF35984 | 0.058 | 0.482 | 0.120 | VL | CDRL3 | T91P | 2144 | 1278 |
| AHF36000 | 0.173 | 0.482 | 0.358 | VL | CDRL3 | T91Q | 2145 | 1278 |
| AHF35976 | 0.189 | 0.482 | 0.392 | VL | CDRL3 | T91R | 2146 | 1278 |
| AHF36006 | 0.329 | 0.482 | 0.683 | VL | CDRL3 | T91S | 2147 | 1278 |
| AHF36012 | 0.060 | 0.482 | 0.124 | VL | CDRL3 | T91V | 2148 | 1278 |
| AHF36002 | 0.055 | 0.482 | 0.113 | VL | CDRL3 | T91W | 2149 | 1278 |
| AHF35990 | 0.155 | 0.482 | 0.321 | VL | CDRL3 | T91Y | 2150 | 1278 |
| AHF36030 | 0.518 | 0.531 | 0.975 | VL | CDRL3 | S92A | 2151 | 1278 |
| AHF36050 | 0.483 | 0.531 | 0.908 | VL | CDRL3 | S92D | 2152 | 1278 |
| AHF36047 | 0.179 | 0.531 | 0.337 | VL | CDRL3 | S92E | 2153 | 1278 |
| AHF36045 | 0.458 | 0.531 | 0.861 | VL | CDRL3 | S92F | 2154 | 1278 |
| AHF36022 | 0.607 | 0.531 | 1.142 | VL | CDRL3 | S92G | 2155 | 1278 |
| AHF36026 | 0.371 | 0.531 | 0.698 | VL | CDRL3 | S92H | 2156 | 1278 |
| AHF36020 | 0.282 | 0.531 | 0.531 | VL | CDRL3 | S92I | 2157 | 1278 |
| AHF36025 | 0.445 | 0.531 | 0.838 | VL | CDRL3 | S92K | 2158 | 1278 |
| AHF36051 | 0.306 | 0.531 | 0.576 | VL | CDRL3 | S92L | 2159 | 1278 |
| AHF36023 | 0.489 | 0.531 | 0.920 | VL | CDRL3 | S92M | 2160 | 1278 |
| AHF36042 | 0.513 | 0.531 | 0.965 | VL | CDRL3 | S92N | 2161 | 1278 |
| AHF36048 | 0.052 | 0.531 | 0.097 | VL | CDRL3 | S92P | 2162 | 1278 |
| AHF36035 | 0.440 | 0.531 | 0.829 | VL | CDRL3 | S92Q | 2163 | 1278 |
| AHF36040 | 0.607 | 0.531 | 1.142 | VL | CDRL3 | S92R | 2164 | 1278 |
| AHF36046 | 0.476 | 0.531 | 0.897 | VL | CDRL3 | S92T | 2165 | 1278 |
| AHF36032 | 0.446 | 0.531 | 0.839 | VL | CDRL3 | S92V | 2166 | 1278 |
| AHF36016 | 0.483 | 0.531 | 0.909 | VL | CDRL3 | S92W | 2167 | 1278 |
| AHF36021 | 0.511 | 0.531 | 0.962 | VL | CDRL3 | S92Y | 2168 | 1278 |
| AHF36103 | 0.582 | 0.531 | 1.095 | VL | CDRL3 | S93A | 2169 | 1278 |
| AHF36071 | 0.303 | 0.531 | 0.571 | VL | CDRL3 | S93D | 2170 | 1278 |
| AHF36073 | 0.447 | 0.531 | 0.840 | VL | CDRL3 | S93E | 2171 | 1278 |
| AHF36077 | 0.547 | 0.531 | 1.029 | VL | CDRL3 | S93F | 2172 | 1278 |
| AHF36085 | 0.277 | 0.531 | 0.522 | VL | CDRL3 | S93G | 2173 | 1278 |
| AHF36086 | 0.531 | 0.531 | 1.000 | VL | CDRL3 | S93H | 2174 | 1278 |
| AHF36087 | 0.584 | 0.531 | 1.098 | VL | CDRL3 | S93K | 2175 | 1278 |
| AHF36083 | 0.448 | 0.531 | 0.842 | VL | CDRL3 | S93L | 2176 | 1278 |
| AHF36061 | 0.452 | 0.531 | 0.850 | VL | CDRL3 | S93M | 2177 | 1278 |
| AHF36097 | 0.445 | 0.531 | 0.837 | VL | CDRL3 | S93N | 2178 | 1278 |
| AHF36078 | 0.536 | 0.531 | 1.009 | VL | CDRL3 | S93P | 2179 | 1278 |
| AHF36093 | 0.428 | 0.531 | 0.806 | VL | CDRL3 | S93Q | 2180 | 1278 |
| AHF36063 | 0.545 | 0.531 | 1.026 | VL | CDRL3 | S93R | 2181 | 1278 |
| AHF36076 | 0.512 | 0.531 | 0.964 | VL | CDRL3 | S93T | 2182 | 1278 |
| AHF36074 | 0.487 | 0.531 | 0.917 | VL | CDRL3 | S93V | 2183 | 1278 |
| AHF36062 | 0.414 | 0.531 | 0.778 | VL | CDRL3 | S93W | 2184 | 1278 |
| AHF36118 | 0.080 | 0.564 | 0.141 | VL | CDRL3 | P94A | 2185 | 1278 |
| AHF36143 | 0.059 | 0.564 | 0.105 | VL | CDRL3 | P94C | 2186 | 1278 |
| AHF36108 | 0.212 | 0.564 | 0.376 | VL | CDRL3 | P94E | 2187 | 1278 |
| AHF36152 | 0.164 | 0.564 | 0.291 | VL | CDRL3 | P94F | 2188 | 1278 |
| AHF36133 | 0.097 | 0.564 | 0.172 | VL | CDRL3 | P94H | 2189 | 1278 |
| AHF36109 | 0.209 | 0.564 | 0.371 | VL | CDRL3 | P94I | 2190 | 1278 |
| AHF36105 | 0.055 | 0.564 | 0.098 | VL | CDRL3 | P94K | 2191 | 1278 |
| AHF36111 | 0.086 | 0.564 | 0.153 | VL | CDRL3 | P94L | 2192 | 1278 |
| AHF36129 | 0.055 | 0.564 | 0.098 | VL | CDRL3 | P94N | 2193 | 1278 |
| AHF36137 | 0.055 | 0.564 | 0.098 | VL | CDRL3 | P94Q | 2194 | 1278 |
| AHF36145 | 0.056 | 0.564 | 0.100 | VL | CDRL3 | P94R | 2195 | 1278 |
| AHF36115 | 0.076 | 0.564 | 0.134 | VL | CDRL3 | P94S | 2196 | 1278 |
| AHF36110 | 0.208 | 0.564 | 0.368 | VL | CDRL3 | P94T | 2197 | 1278 |
| AHF36120 | 0.054 | 0.564 | 0.096 | VL | CDRL3 | P94V | 2198 | 1278 |
| AHF36117 | 0.220 | 0.564 | 0.391 | VL | CDRL3 | P94W | 2199 | 1278 |
| AHF36125 | 0.209 | 0.564 | 0.370 | VL | CDRL3 | P94Y | 2200 | 1278 |
| AHF36188 | 0.277 | 0.564 | 0.490 | VL | CDRL3 | F95A | 2201 | 1278 |
| AHF36166 | 0.207 | 0.564 | 0.367 | VL | CDRL3 | F95E | 2202 | 1278 |
| AHF36171 | 0.221 | 0.564 | 0.391 | VL | CDRL3 | F95G | 2203 | 1278 |
| AHF36161 | 0.150 | 0.564 | 0.266 | VL | CDRL3 | F95H | 2204 | 1278 |
| AHF36154 | 0.245 | 0.564 | 0.434 | VL | CDRL3 | F95I | 2205 | 1278 |
| AHF36178 | 0.534 | 0.564 | 0.946 | VL | CDRL3 | F95L | 2206 | 1278 |
| AHF36162 | 0.448 | 0.564 | 0.794 | VL | CDRL3 | F95M | 2207 | 1278 |
| AHF36167 | 0.258 | 0.564 | 0.458 | VL | CDRL3 | F95N | 2208 | 1278 |
| AHF36192 | 0.458 | 0.564 | 0.812 | VL | CDRL3 | F95P | 2209 | 1278 |
| AHF36172 | 0.125 | 0.564 | 0.222 | VL | CDRL3 | F95Q | 2210 | 1278 |
| AHF36168 | 0.090 | 0.564 | 0.159 | VL | CDRL3 | F95R | 2211 | 1278 |
| AHF36163 | 0.247 | 0.564 | 0.438 | VL | CDRL3 | F95S | 2212 | 1278 |
| AHF36159 | 0.411 | 0.564 | 0.729 | VL | CDRL3 | F95V | 2213 | 1278 |
| AHF36190 | 0.319 | 0.564 | 0.566 | VL | CDRL3 | F95W | 2214 | 1278 |
| AHF36198 | 0.195 | 0.443 | 0.441 | VL | CDRL3 | T96D | 2215 | 1278 |
| AHF36244 | 0.322 | 0.443 | 0.726 | VL | CDRL3 | T96E | 2216 | 1278 |
| AHF36201 | 0.379 | 0.443 | 0.856 | VL | CDRL3 | T96F | 2217 | 1278 |
| AHF36216 | 0.205 | 0.443 | 0.462 | VL | CDRL3 | T96G | 2218 | 1278 |
| AHF36225 | 0.493 | 0.443 | 1.112 | VL | CDRL3 | T96H | 2219 | 1278 |
| AHF36230 | 0.374 | 0.443 | 0.845 | VL | CDRL3 | T96I | 2220 | 1278 |
| AHF36199 | 0.245 | 0.443 | 0.553 | VL | CDRL3 | T96K | 2221 | 1278 |
| AHF36228 | 0.380 | 0.443 | 0.857 | VL | CDRL3 | T96L | 2222 | 1278 |
| AHF36209 | 0.326 | 0.443 | 0.737 | VL | CDRL3 | T96M | 2223 | 1278 |
| AHF36197 | 0.328 | 0.443 | 0.742 | VL | CDRL3 | T96Q | 2224 | 1278 |
| AHF36213 | 0.351 | 0.443 | 0.791 | VL | CDRL3 | T96R | 2225 | 1278 |
| AHF36205 | 0.512 | 0.443 | 1.156 | VL | CDRL3 | T96S | 2226 | 1278 |
| AHF36210 | 0.668 | 0.443 | 1.507 | VL | CDRL3 | T96V | 2227 | 1278 |
| AHF36207 | 0.370 | 0.443 | 0.836 | VL | CDRL3 | T96W | 2228 | 1278 |
| AHF36237 | 0.464 | 0.443 | 1.047 | VL | CDRL3 | T96Y | 2229 | 1278 |

TABLE 28
Mutant Fab Binding to Human TCRβV12-3 by Biacore (SPR)

| | | | | | | | | Ratio= WT | Ratio= WT | Fab | |
|---|---|---|---|---|---|---|---|---|---|---|---|
| | | | Mutation | | | | Rmax | (kd)/Mutant | (KD)/Mutant | SEQ ID | |
| Sequence | Fab ID | Mutation | location | ka (1/Ms) | kd (1/s) | KD (M) | (RU) | (kd) | (KD) | HC | LC |
| | WT | — | — | 5.75E+04 | 1.48E−03 | 2.58E−08 | 27.4 | 1.00 | 1.00 | 1278 | 1280 |
| AHF34274 | AHF31301 | N31Y | CDRH1 | 7.36E+04 | 1.35E−03 | 1.84E−08 | 23.5 | 1.10 | 1.40 | 1426 | 1280 |
| AHF34278 | AHF31302 | N31R | CDRH1 | 7.23E+04 | 7.05E−04 | 9.75E−09 | 56.3 | 2.10 | 2.65 | 1427 | 1280 |
| AHF34296 | AHF31304 | N31I | CDRH1 | 6.94E+04 | 6.66E−04 | 9.60E−09 | 35.5 | 2.22 | 2.69 | 1431 | 1280 |
| AHF34313 | AHF31306 | F32K | CDRH1 | 7.17E+04 | 1.03E−03 | 1.43E−08 | 35.5 | 1.44 | 1.80 | 1432 | 1280 |
| AHF34353 | AHF31310 | F32T | CDRH1 | 6.59E+04 | 5.83E−04 | 8.85E−09 | 85.8 | 2.54 | 2.92 | 1433 | 1280 |
| AHF34452 | AHF31311 | G54S | CDRH2 | 7.84E+04 | 4.29E−04 | 5.47E−09 | 69.8 | 3.45 | 4.72 | 1434 | 1280 |
| AHF34459 | AHF31312 | G54W | CDRH2 | 5.96E+04 | 4.69E−04 | 7.88E−09 | 20.4 | 3.16 | 3.27 | 1435 | 1280 |
| AHF34461 | AHF31313 | G54Y | CDRH2 | 6.61E+04 | 8.12E−04 | 1.23E−08 | 47.8 | 1.82 | 2.10 | 1436 | 1280 |
| AHF34491 | AHF31315 | G54T | CDRH2 | 7.27E+04 | 5.13E−04 | 7.06E−09 | 50.4 | 2.88 | 3.65 | 1437 | 1280 |
| AHF34523 | AHF31316 | S55H | CDRH2 | 6.29E+04 | 1.71E−03 | 2.73E−08 | 31.9 | 0.87 | 0.95 | 1438 | 1280 |
| AHF34616 | AHF31317 | T57V | CDRH2 | 5.76E+04 | 1.18E−03 | 2.05E−08 | 38.2 | 1.25 | 1.26 | 1439 | 1280 |
| AHF34646 | AHF31318 | I58F | CDRH2 | 5.79E+04 | 1.59E−03 | 2.75E−08 | 26.7 | 0.93 | 0.94 | 1440 | 1280 |
| AHF34706 | AHF31319 | Y59L | CDRH2 | 6.63E+04 | 5.66E−04 | 8.53E−09 | 79.2 | 2.61 | 3.02 | 1441 | 1280 |
| AHF33544 | AHF31320 | A61K | CDRH2 | 6.18E+04 | 9.65E−04 | 1.56E−08 | 62.6 | 1.53 | 1.65 | 1442 | 1280 |
| AHF33558 | AHF31321 | A61R | CDRH2 | 6.20E+04 | 1.26E−03 | 2.02E−08 | 38.6 | 1.17 | 1.28 | 1443 | 1280 |
| AHF34731 | AHF31322 | T63K | CDRH2 | 6.32E+04 | 9.16E−04 | 1.45E−08 | 63.3 | 1.62 | 1.78 | 1444 | 1280 |
| AHF34881 | AHF31325 | E101T | CDRH3 | 6.62E+04 | 1.58E−03 | 2.39E−08 | 24.3 | 0.94 | 1.08 | 1445 | 1280 |
| AHF34897 | AHF31326 | E101L | CDRH3 | 7.96E+04 | 1.71E−03 | 2.15E−08 | 23.9 | 0.87 | 1.20 | 1446 | 1280 |
| AHF34898 | AHF31327 | E101H | CDRH3 | 6.14E+04 | 1.05E−03 | 1.71E−08 | 42.7 | 1.41 | 1.51 | 1447 | 1280 |
| AHF34904 | AHF31328 | E101A | CDRH3 | 6.56E+04 | 1.00E−03 | 1.52E−08 | 47.1 | 1.48 | 1.70 | 1448 | 1280 |
| AHF35097 | AHF31329 | Y106N | CDRH3 | 5.48E+04 | 1.27E−03 | 2.32E−08 | 39.7 | 1.17 | 1.11 | 1449 | 1280 |
| AHF35102 | AHF31330 | Y106A | CDRH3 | 6.02E+04 | 1.41E−03 | 2.34E−08 | 33.0 | 1.05 | 1.10 | 1450 | 1280 |
| AHF35107 | AHF31332 | Y106W | CDRH3 | 5.97E+04 | 1.21E−03 | 2.02E−08 | 43.2 | 1.22 | 1.28 | 1451 | 1428 |
| AHF35164 | AHF31333 | S26M | CDRL1 | 6.67E+04 | 7.82E−04 | 1.17E−08 | 75.2 | 1.89 | 2.21 | 1278 | 1429 |
| AHF35188 | AHF31334 | S27K | CDRL1 | 6.44E+04 | 7.39E−04 | 1.15E−08 | 61.4 | 2.00 | 2.24 | 1278 | 1287 |
| AHF35230 | AHF31335 | S27R | CDRL1 | 6.54E+04 | 6.68E−04 | 1.02E−08 | 80.3 | 2.22 | 2.53 | 1278 | 1430 |
| AHF35238 | AHF31336 | S28K | CDRL1 | 7.16E+04 | 1.70E−03 | 2.38E−08 | 25.3 | 0.87 | 1.08 | 1278 | 1452 |
| AHF35445 | AHF31337 | I32A | CDRL1 | 6.38E+04 | 5.40E−04 | 8.46E−09 | 43.1 | 2.74 | 3.05 | 1278 | 1291 |
| AHF35605 | AHF31339 | S51N | CDRL2 | 5.82E+04 | 2.00E−03 | 3.43E−08 | 22.7 | 0.74 | 0.75 | 1278 | 1453 |
| AHF35625 | AHF31341 | S51K | CDRL2 | 6.14E+04 | 1.64E−03 | 2.67E−08 | 25.9 | 0.90 | 0.97 | 1278 | 1454 |
| AHF35650 | AHF31343 | N52R | CDRL2 | 5.46E+04 | 9.23E−04 | 1.69E−08 | 57.2 | 1.60 | 1.53 | 1278 | 1455 |
| AHF35675 | AHF31344 | N52K | CDRL2 | 5.17E+04 | 1.75E−03 | 3.40E−08 | 24.6 | 0.85 | 0.76 | 1278 | 1456 |
| AHF35760 | AHF31345 | A54R | CDRL2 | 6.47E+04 | 1.59E−03 | 2.46E−08 | 30.6 | 0.93 | 1.05 | 1278 | 1457 |
| AHF35792 | AHF31346 | P55R | CDRL2 | 5.58E+04 | 9.43E−04 | 1.69E−08 | 49.0 | 1.57 | 1.53 | 1278 | 1458 |
| | Blank | — | | NA | NA | NA | NA | | | | |
| | Negative | — | | NA | NA | NA | NA | | | | |
| | control | | | | | | | | | | |

TABLE 29
Combinatorial Mutant Fab Binding to Human TCRβV12-3 by ELISA
Human TCRβV12-3-binding ELISA

| | | | | | Ratio = | | |
|---|---|---|---|---|---|---|---|
| | | | | | OD | Fab SEQ | |
| | Mutations | | Mutant | WT | (Mutant)/OD | ID NO | |
| Sequence ID | VH | VL | OD450nm | OD450nm | (WT) | VH | VL |
| WT | — | — | — | 0.34 | — | 1278 | 1280 |
| AHF40573 | N31I G54T Y59L | S27R I32A T96V | 1.414 | 0.457 | 3.10 | 1296 | 1284 |
| AHF40574 | N31I G54T Y59L | I32A T96V | 1.383 | 0.457 | 3.03 | 1296 | 1285 |
| AHF40575 | N31R F32T G54S Y59L | S27R I32A T96V | 1.465 | 0.457 | 3.21 | 1297 | 1284 |
| AHF40576 | N31I F32K E101A | S27K I32A T96V | 1.469 | 0.457 | 3.21 | 1298 | 1286 |
| AHF40577 | F32K G54S | S27K | 1.379 | 0.457 | 3.02 | 1299 | 1287 |
| AHF40578 | G54W Y59L E101A | I32A T96V | 1.424 | 0.457 | 3.12 | 1300 | 1285 |
| AHF40579 | N31I G54W | S27R I32A | 1.429 | 0.457 | 3.13 | 1301 | 1288 |
| AHF40580 | N31R G54W Y59L | S27K T96V | 1.369 | 0.457 | 2.99 | 1302 | 1289 |
| AHF40581 | N31R F32K G54T Y59L | S27R T96V | 1.248 | 0.455 | 2.745 | 1303 | 1290 |
| AHF40582 | N31I G54W E101A | I32A T96V | 1.238 | 0.455 | 2.722 | 1304 | 1285 |
| AHF40583 | N31I G54S Y59L E101A | I32A T96V | 1.296 | 0.455 | 2.850 | 1305 | 1285 |
| AHF40584 | F32K G54S Y59L E101A | I32A | 1.218 | 0.455 | 2.678 | 1306 | 1291 |
| AHF40585 | N31R G54T | S27R I32A | 1.255 | 0.455 | 2.760 | 1307 | 1288 |
| AHF40586 | N31I F32T G54W E101A | S27K T96V | 1.259 | 0.455 | 2.770 | 1308 | 1289 |
| AHF40587 | N31R F32K E101A | I32A | 1.222 | 0.455 | 2.688 | 1309 | 1291 |
| AHF40588 | N31I F32T G54T Y59L | S27K | 1.246 | 0.455 | 2.741 | 1310 | 1287 |
| AHF40589 | N31I F32K G54S Y59L E101A | S27K T96V | 1.224 | 0.462 | 2.647 | 1311 | 1289 |
| AHF40590 | N31R F32K G54W E101A | I32A | 1.300 | 0.462 | 2.813 | 1312 | 1291 |
| AHF40591 | N31I G54T Y59L | S27R I32A | 1.310 | 0.462 | 2.835 | 1296 | 1288 |
| AHF40593 | N31I F32Y G54T Y59L E101A | I32A | 1.326 | 0.462 | 2.869 | 1313 | 1291 |
| AHF40594 | N31R F32T Y59L | S27K I32A T96V | 1.219 | 0.462 | 2.636 | 1314 | 1286 |
| AHF40595 | F32T G54T Y59L E101A | S27R I32A | 1.304 | 0.462 | 2.821 | 1315 | 1288 |
| AHF40596 | F32K Y59L | S27K I32A | 1.245 | 0.462 | 2.694 | 1316 | 1292 |
| AHF40597 | N31I F32T G54T Y59L E101A | S27R T96V | 1.192 | 0.496 | 2.402 | 1317 | 1290 |
| AHF40600 | N31R G54T | S27R I32A T96V | 1.191 | 0.496 | 2.400 | 1307 | 1284 |
| AHF40601 | N31I F32T G54S E101A | S27K I32A T96V | 1.219 | 0.496 | 2.455 | 1319 | 1286 |
| AHF40602 | N31R F32T G54W | I32A T96V | 1.215 | 0.496 | 2.448 | 1320 | 1285 |
| AHF40603 | N31R F32K G54W E101A | S27K T96V | 1.198 | 0.496 | 2.414 | 1312 | 1289 |
| AHF40604 | G54W E101A | S27K T96V | 1.151 | 0.496 | 2.318 | 1321 | 1289 |
| AHF40605 | F32T G54T Y59L | S27R I32A T96V | 1.210 | 0.440 | 2.752 | 1322 | 1284 |
| AHF40606 | F32T G54S Y59L E101A | S27R I32A T96V | 1.224 | 0.440 | 2.784 | 1323 | 1284 |
| AHF40607 | N31R F32T G54W Y59L E101A | S27R I32A T96V | 1.209 | 0.440 | 2.749 | 1324 | 1284 |
| AHF40608 | F32K G54W Y59L E101A | I32A | 1.180 | 0.440 | 2.685 | 1325 | 1291 |
| AHF40609 | F32K Y59L E101A | S27R I32A T96V | 1.223 | 0.440 | 2.782 | 1326 | 1284 |
| AHF40610 | N31R F32T G54S Y59L | S27K I32A T96V | 1.215 | 0.440 | 2.764 | 1297 | 1286 |
| AHF40611 | F32T G54S E101A | S27R I32A T96V | 1.186 | 0.440 | 2.698 | 1327 | 1284 |
| AHF40612 | N31I F32T G54S Y59L | S27R T96V | 1.185 | 0.440 | 2.694 | 1328 | 1290 |

TABLE 30
Combinatorial Mutant Fab Binding to Human TCRβV12-3 by Biacore
Human TCRβV12-3-binding by Biacore (SPR)

| | | | | | | | Ratio= | Ratio= |
|---|---|---|---|---|---|---|---|---|
| | Mutations | | | | | Rmax | WT(kd)/Mutant | WT(KD)/Mutant |
| Sequence ID | VH | VL | ka (1/Ms) | kd (1/s) | KD (M) | (RU) | (kd) | (KD) |
| WT | — | — | 4.68E+04 | 9.09E−04 | 1.94E−08 | 59.6 | — | — |
| AHF40573 | N31I G54T Y59L | S27R I32A T96V | 4.23E+04 | 1.16E−04 | 2.73E−09 | 33.9 | 7.84 | 7.1 |
| AHF40574 | N31I G54T Y59L | I32A T96V | 4.08E+04 | 1.40E−04 | 3.43E−09 | 34.1 | 6.49 | 5.7 |
| AHF40575 | N31R F32T G54S Y59L | S27R I32A T96V | 3.71E+04 | 1.33E−04 | 3.59E−09 | 40.6 | 6.83 | 5.4 |
| AHF40576 | N31I F32K E101A | S27K I32A T96V | 3.63E+04 | 8.85E−05 | 2.44E−09 | 34.4 | 10.27 | 8.0 |
| AHF40577 | F32K G54S | S27K | 5.24E+04 | 2.21E−04 | 4.22E−09 | 69.0 | 4.11 | 4.6 |
| AHF40578 | G54W Y59L E101A | I32A T96V | 2.46E+04 | 1.11E−04 | 4.53E−09 | 20.4 | 8.19 | 4.3 |
| AHF40579 | N31I G54W | S27R I32A | 3.29E+04 | 3.46E−04 | 1.05E−08 | 15.2 | 2.63 | 1.8 |
| AHF40580 | N31R G54W Y59L | S27K T96V | 2.98E+04 | 2.34E−04 | 7.84E−09 | 18.3 | 3.88 | 2.5 |
| AHF40581 | N31R F32K G54T Y59L | S27R T96V | 3.91E+04 | 7.04E−05 | 1.80E−09 | 45.3 | 12.91 | 10.8 |
| AHF40582 | N31I G54W E101A | I32A T96V | 2.26E+04 | 3.18E−04 | 1.40E−08 | 10.3 | 2.86 | 1.4 |
| AHF40583 | N31I G54S Y59L E101A | I32A T96V | 3.48E+04 | 1.48E−04 | 4.26E−09 | 28.2 | 6.14 | 4.6 |
| AHF40584 | F32K G54S Y59L E101A | I32A | 2.86E+04 | 1.53E−04 | 5.37E−09 | 28.5 | 5.94 | 3.6 |
| AHF40585 | N31R G54T | S27R I32A | 4.82E+04 | 7.43E−05 | 1.54E−09 | 46.3 | 12.23 | 12.6 |
| AHF40586 | N31I F32T G54W E101A | S27K T96V | 3.07E+04 | 3.24E−04 | 1.06E−08 | 15.6 | 2.81 | 1.8 |
| AHF40587 | N31R F32K E101A | I32A | 2.72E+04 | 2.11E−04 | 7.75E−09 | 37.7 | 4.31 | 2.5 |
| AHF40588 | N31I F32T G54T Y59L | S27K | 4.63E+04 | 6.62E−05 | 1.43E−09 | 33.4 | 13.73 | 13.6 |
| AHF40589 | N31I F32K G54S Y59L | S27K T96V | 3.69E+04 | 5.74E−05 | 1.56E−09 | 27.6 | 15.84 | 12.4 |
| | E101A | | | | | | | |
| AHF40590 | N31R F32K G54W E101A | I32A | 1.83E+04 | 1.62E−04 | 8.87E−09 | 13.0 | 5.61 | 2.2 |
| AHF40591 | N31I G54T Y59L | S27R I32A | 4.08E+04 | 6.74E−05 | 1.65E−09 | 31.2 | 13.49 | 11.8 |
| AHF40593 | N31I F32Y G54T Y59L | I32A | 3.76E+04 | 9.58E−05 | 2.55E−09 | 33.6 | 9.49 | 7.6 |
| | E101A | | | | | | | |
| AHF40594 | N31R F32T Y59L | S27K I32A T96V | 3.40E+04 | 3.97E−05 | 1.17E−09 | 54.1 | 22.90 | 16.6 |
| AHF40595 | F32T G54T Y59L E101A | S27R I32A | 3.47E+04 | 5.02E−06 | 1.45E−10 | 64.4 | 181.08 | 133.8 |
| AHF40596 | F32K Y59L | S27K I32A | 3.50E+04 | 8.98E−05 | 2.57E−09 | 40.6 | 10.12 | 7.5 |
| AHF40597 | N31I F32T G54T Y59L | S27R T96V | 4.37E+04 | 1.43E−05 | 3.28E−10 | 59.4 | 63.57 | 59.1 |
| | E101A | | | | | | | |
| AHF40600 | N31R G54T | S27R I32A T96V | 5.47E+04 | 4.72E−05 | 8.62E−10 | 64.2 | 19.26 | 22.5 |
| AHF40601 | N31I F32T G54S E101A | S27K I32A T96V | 5.00E+04 | 5.36E−05 | 1.07E−09 | 50.3 | 16.96 | 18.1 |
| AHF40602 | N31R F32T G54W | I32A T96V | 3.20E+04 | 1.40E−04 | 4.40E−09 | 23.3 | 6.49 | 4.4 |
| AHF40603 | N31R F32K G54W E101A | S27K T96V | 2.01E+04 | 2.33E−04 | 1.16E−08 | 15.1 | 3.90 | 1.7 |
| AHF40604 | G54W E101A | S27K T96V | 4.12E+04 | 1.54E−04 | 3.74E−09 | 37.9 | 5.90 | 5.2 |
| AHF40605 | F32T G54T Y59L | S27R I32A T96V | 4.04E+04 | 8.71E−06 | 2.15E−10 | 77.4 | 104.36 | 90.2 |
| AHF40606 | F32T G54S Y59L E101A | S27R I32A T96V | 4.38E+04 | 2.43E−06 | 5.55E−11 | 60.2 | 374.07 | 349.5 |
| AHF40607 | N31R F32T G54W Y59L | S27R I32A T96V | 2.14E+04 | 1.43E−04 | 6.69E−09 | 20.2 | 6.36 | 2.9 |
| | E101A | | | | | | | |
| AHF40608 | F32K G54W Y59L E101A | I32A | 2.08E+04 | 7.11E−05 | 3.42E−09 | 27.6 | 12.78 | 5.7 |
| AHF40609 | F32K Y59L E101A | S27R I32A T96V | 2.97E+04 | 7.48E−05 | 2.52E−09 | 38.1 | 12.15 | 7.7 |
| AHF40610 | N31R F32T G54S Y59L | S27K I32A T96V | 3.78E+04 | 1.31E−05 | 3.45E−10 | 58.7 | 69.39 | 56.2 |
| AHF40611 | F32T G54S E101A | S27R I32A T96V | 4.81E+04 | 1.11E−05 | 2.31E−10 | 95.2 | 81.89 | 84.0 |
| AHF40612 | N31I F32T G54S Y59L | S27R T96V | 5.34E+04 | 4.45E−06 | 8.34E−11 | 57.4 | 204.27 | 232.6 |
| Negative | — | — | NA | NA | NA | NA | — | — |
| control | | | | | | | | |
| Blank | — | — | NA | NA | NA | NA | — | — |

TABLE 31
Exemplary TCRβV12-3-binding Domains

| | | | Kabat CDRs | | | | | | IMGT CDRs | | | | | |
|---|---|---|---|---|---|---|---|---|---|---|---|---|---|---|
| | | | CDR | CDR | CDR | CDR | CDR | CDR | CDR | CDR | CDR | CDR | CDR | CDR |
| ID | VL | VH | L1 | L2 | L3 | H1 | H2 | H3 | L1 | L2 | L3 | H1 | H2 | H3 |
| Wildtype | 1100 | 1113 | 1148 | 1156 | 1157 | 1160 | 1171 | 1180 | 1182 | YTS | 1157 | 1186 | 1197 | 1201 |
| AHF10008 | 1100 | 1114 | 1148 | 1156 | 1157 | 1160 | 1171 | 1180 | 1182 | YTS | 1157 | 1186 | 1197 | 1201 |
| AHF10009 | 1101 | 1114 | 1148 | 1156 | 1157 | 1160 | 1171 | 1180 | 1182 | YTS | 1157 | 1186 | 1197 | 1201 |
| AHF10010 | 1102 | 1114 | 1149 | 1156 | 1158 | 1160 | 1171 | 1180 | 1183 | YTS | 1158 | 1186 | 1197 | 1201 |
| AHF10011 | 1103 | 1114 | 1150 | 1156 | 1158 | 1160 | 1171 | 1180 | 1183 | YTS | 1158 | 1186 | 1197 | 1201 |
| AHF10012 | 1103 | 1115 | 1150 | 1156 | 1158 | 1161 | 1172 | 1180 | 1183 | YTS | 1158 | 1187 | 1197 | 1201 |
| AHF40573 | 1104 | 1116 | 1151 | 1156 | 1159 | 1162 | 1173 | 1180 | 1184 | YTS | 1159 | 1188 | 1198 | 1201 |
| AHF40574 | 1105 | 1116 | 1148 | 1156 | 1159 | 1162 | 1173 | 1180 | 1182 | YTS | 1159 | 1188 | 1198 | 1201 |
| AHF40575 | 1104 | 1117 | 1151 | 1156 | 1159 | 1163 | 1174 | 1180 | 1184 | YTS | 1159 | 1189 | 1199 | 1201 |
| AHF40576 | 1106 | 1118 | 1152 | 1156 | 1159 | 1164 | 1171 | 1181 | 1185 | YTS | 1159 | 1190 | 1197 | 1202 |
| AHF40577 | 1107 | 1119 | 1153 | 1156 | 1157 | 1165 | 1175 | 1180 | 1185 | YTS | 1157 | 1191 | 1199 | 1201 |
| AHF40578 | 1105 | 1120 | 1154 | 1156 | 1159 | 1160 | 1176 | 1181 | 1182 | YTS | 1159 | 1186 | 1200 | 1202 |
| AHF40579 | 1108 | 1121 | 1151 | 1156 | 1157 | 1162 | 1177 | 1180 | 1184 | YTS | 1157 | 1188 | 1200 | 1201 |
| AHF40580 | 1109 | 1122 | 1153 | 1156 | 1159 | 1166 | 1176 | 1180 | 1185 | YTS | 1159 | 1192 | 1200 | 1201 |
| AHF40581 | 1110 | 1123 | 1155 | 1156 | 1159 | 1167 | 1173 | 1180 | 1184 | YTS | 1159 | 1193 | 1198 | 1201 |
| AHF40582 | 1105 | 1124 | 1154 | 1156 | 1159 | 1162 | 1177 | 1181 | 1182 | YTS | 1159 | 1188 | 1200 | 1202 |
| AHF40583 | 1105 | 1125 | 1154 | 1156 | 1159 | 1162 | 1174 | 1181 | 1182 | YTS | 1159 | 1188 | 1199 | 1202 |
| AHF40584 | 1111 | 1126 | 1154 | 1156 | 1157 | 1165 | 1174 | 1181 | 1182 | YTS | 1157 | 1191 | 1199 | 1202 |
| AHF40585 | 1108 | 1127 | 1151 | 1156 | 1157 | 1166 | 1178 | 1180 | 1184 | YTS | 1157 | 1192 | 1198 | 1201 |
| AHF40586 | 1109 | 1128 | 1153 | 1156 | 1159 | 1168 | 1173 | 1181 | 1185 | YTS | 1159 | 1194 | 1198 | 1202 |
| AHF40587 | 1111 | 1129 | 1154 | 1156 | 1157 | 1167 | 1171 | 1181 | 1182 | YTS | 1157 | 1193 | 1197 | 1202 |
| AHF40588 | 1107 | 1130 | 1153 | 1156 | 1157 | 1168 | 1177 | 1181 | 1185 | YTS | 1157 | 1194 | 1200 | 1202 |
| AHF40589 | 1109 | 1131 | 1153 | 1156 | 1159 | 1164 | 1174 | 1181 | 1185 | YTS | 1159 | 1190 | 1199 | 1202 |
| AHF40590 | 1111 | 1132 | 1154 | 1156 | 1157 | 1167 | 1177 | 1181 | 1182 | YTS | 1157 | 1193 | 1200 | 1202 |
| AHF40591 | 1108 | 1116 | 1151 | 1156 | 1157 | 1162 | 1173 | 1180 | 1184 | YTS | 1157 | 1188 | 1198 | 1201 |
| AHF40593 | 1111 | 1133 | 1154 | 1156 | 1157 | 1169 | 1173 | 1181 | 1182 | YTS | 1157 | 1195 | 1198 | 1202 |
| AHF40594 | 1106 | 1134 | 1152 | 1156 | 1159 | 1163 | 1179 | 1180 | 1185 | YTS | 1159 | 1189 | 1197 | 1201 |
| AHF40595 | 1108 | 1135 | 1151 | 1156 | 1157 | 1170 | 1173 | 1181 | 1184 | YTS | 1157 | 1196 | 1198 | 1202 |
| AHF40596 | 1112 | 1136 | 1152 | 1156 | 1157 | 1165 | 1179 | 1180 | 1185 | YTS | 1157 | 1191 | 1197 | 1201 |
| AHF40597 | 1110 | 1137 | 1155 | 1156 | 1159 | 1168 | 1173 | 1181 | 1184 | YTS | 1159 | 1194 | 1198 | 1202 |
| AHF40600 | 1104 | 1127 | 1151 | 1156 | 1159 | 1166 | 1178 | 1180 | 1184 | YTS | 1159 | 1192 | 1198 | 1201 |
| AHF40601 | 1106 | 1138 | 1152 | 1156 | 1159 | 1168 | 1175 | 1181 | 1185 | YTS | 1159 | 1194 | 1199 | 1202 |
| AHF40602 | 1105 | 1139 | 1154 | 1156 | 1159 | 1163 | 1177 | 1180 | 1182 | YTS | 1159 | 1189 | 1200 | 1201 |
| AHF40603 | 1109 | 1132 | 1153 | 1156 | 1159 | 1167 | 1177 | 1181 | 1185 | YTS | 1159 | 1193 | 1200 | 1202 |
| AHF40604 | 1109 | 1140 | 1153 | 1156 | 1159 | 1160 | 1177 | 1181 | 1185 | YTS | 1159 | 1186 | 1200 | 1202 |
| AHF40605 | 1104 | 1141 | 1151 | 1156 | 1159 | 1170 | 1173 | 1180 | 1184 | YTS | 1159 | 1196 | 1198 | 1201 |
| AHF40606 | 1104 | 1142 | 1151 | 1156 | 1159 | 1170 | 1174 | 1181 | 1184 | YTS | 1159 | 1196 | 1199 | 1202 |
| AHF40607 | 1104 | 1143 | 1151 | 1156 | 1159 | 1163 | 1176 | 1181 | 1184 | YTS | 1159 | 1189 | 1200 | 1202 |
| AHF40608 | 1111 | 1144 | 1154 | 1156 | 1157 | 1165 | 1176 | 1181 | 1182 | YTS | 1157 | 1191 | 1200 | 1202 |
| AHF40609 | 1104 | 1145 | 1151 | 1156 | 1159 | 1165 | 1179 | 1181 | 1184 | YTS | 1159 | 1191 | 1197 | 1202 |
| AHF40610 | 1106 | 1117 | 1152 | 1156 | 1159 | 1163 | 1174 | 1180 | 1185 | YTS | 1159 | 1189 | 1199 | 1201 |
| AHF40611 | 1104 | 1146 | 1151 | 1156 | 1159 | 1170 | 1175 | 1181 | 1184 | YTS | 1159 | 1196 | 1199 | 1202 |
| AHF40612 | 1110 | 1147 | 1155 | 1156 | 1159 | 1168 | 1174 | 1180 | 1184 | YTS | 1159 | 1194 | 1199 | 1201 |

TABLE 32
Exemplary TCRβV12-3 scFv Polypeptide Molecules

| | SEQ |
|---|---|
| ID | ID NO |
| CLY-059 | 1203 |
| CLY-060 | 1204 |
| CLY-061 | 1205 |
| CLY-062 | 1206 |
| CLY-063 | 1207 |
| CLY-064 | 1208 |
| CLY-065 | 1209 |
| CLY-066 | 1210 |
| CLY-067 | 1211 |
| CLY-068 | 1212 |
| CLY-069 | 1213 |
| CLY-070 | 1214 |
| CLY-071 | 1215 |
| CLY-072 | 1216 |
| CLY-073 | 1217 |
| CLY-074 | 1218 |
| CLY-075 | 1219 |
| CLY-076 | 1220 |
| CLY-077 | 1221 |
| CLY-078 | 1222 |
| CLY-079 | 1223 |
| CLY-080 | 1224 |
| CLY-081 | 1225 |
| CLY-082 | 1226 |
| CLY-083 | 1227 |
| CLY-084 | 1228 |
| CLY-085 | 1229 |
| CLY-086 | 1230 |
| CLY-087 | 1231 |
| CLY-088 | 1232 |
| CLY-089 | 1233 |
| CLY-090 | 1234 |
| CLY-091 | 1235 |
| CLY-092 | 1236 |
| CLY-093 | 1237 |
| CLY-094 | 1238 |
| CLY-095 | 1239 |
| CLY-096 | 1240 |
| CLY-097 | 1241 |
| CLY-098 | 1242 |
| CLY-099 | 1243 |
| CLY-100 | 1244 |
| CLY-101 | 1245 |
| CLY-102 | 1246 |
| CLY-103 | 1247 |
| CLY-104 | 1248 |
| CLY-105 | 1249 |
| CLY-106 | 1250 |
| CLY-107 | 1251 |
| CLY-108 | 1252 |
| CLY-109 | 1253 |
| CLY-110 | 1254 |
| CLY-111 | 1255 |
| CLY-112 | 1256 |
| CLY-113 | 1257 |
| CLY-114 | 1258 |
| CLY-115 | 1259 |
| CLY-116 | 1260 |
| CLY-117 | 1261 |
| CLY-118 | 1262 |
| CLY-119 | 1263 |
| CLY-120 | 1264 |
| CLY-121 | 1265 |
| CLY-122 | 1266 |
| CLY-123 | 1267 |
| CLY-124 | 1268 |
| CLY-125 | 1269 |
| CLY-126 | 1270 |
| CLY-127 | 1271 |
| CLY-128 | 1272 |
| CLY-129 | 1273 |
| CLY-130 | 1274 |
| CLY-131 | 1275 |
| CLY-132 | 1276 |
| CLY-133 | 1277 |

TABLE 33
Exemplary TCRβV12-3 Fab Polypeptide Molecules

| | SEQ ID NO | | | SEQ ID NO | |
|---|---|---|---|---|---|
| ID | LC | HC | ID | LC | HC |
| CLY-034 | 1280 | 1293 | CLY-053 | 1287 | 1310 |
| CLY-034-2 | 1280 | 1294 | CLY-054 | 1289 | 1311 |
| CLY-034-3 | 1281 | 1294 | CLY-055 | 1291 | 1312 |
| CLY-034-4 | 1282 | 1294 | CLY-056 | 1288 | 1296 |
| CLY-034-5 | 1283 | 1294 | CLY-057 | 1291 | 1313 |
| CLY-034-6 | 1283 | 1295 | CLY-058 | 1286 | 1314 |
| CLY-038 | 1284 | 1296 | CLY-035 | 1288 | 1315 |
| CLY-039 | 1285 | 1296 | CLY-035-2 | 1292 | 1316 |
| CLY-040 | 1284 | 1297 | CLY-035-3 | 1290 | 1317 |
| CLY-041 | 1286 | 1298 | CLY-035-4 | 1284 | 1307 |
| CLY-042 | 1287 | 1299 | CLY-035-5 | 1286 | 1319 |
| CLY-043 | 1285 | 1300 | CLY-035-6 | 1285 | 1320 |
| CLY-044 | 1288 | 1301 | CLY-035-7 | 1289 | 1312 |
| CLY-045 | 1289 | 1302 | CLY-035-8 | 1289 | 1321 |
| CLY-046 | 1290 | 1303 | CLY-035-9 | 1284 | 1322 |
| CLY-047 | 1285 | 1304 | CLY-036 | 1284 | 1323 |
| CLY-048 | 1285 | 1305 | CLY-036-2 | 1284 | 1324 |
| CLY-049 | 1291 | 1306 | CLY-036-3 | 1291 | 1325 |
| CLY-050 | 1288 | 1307 | CLY-036-4 | 1284 | 1326 |
| CLY-051 | 1289 | 1308 | CLY-036-5 | 1286 | 1297 |
| CLY-052 | 1291 | 1309 | CLY-036-6 | 1284 | 1327 |
| | | | CLY-037 | 1290 | 1328 |

### Example 4: Design and Generation of Polypeptide Molecules Bispecific to CD19 and TCRβV6-5

The objective of this study was to design and evaluate polypeptide molecules bispecific to CD19 and TCRβV6-5.

Exemplary bispecific polypeptide molecules utilizing the Fab fused to scFv configuration with a knob-in-hole Fc for half-life extension were generated as shown in FIG. 19B and Table 34 below.

TABLE 34
Anti-CD19/Anti-TCRβV6-5 Polypeptide Molecules

| | | | SEQ ID NO | | |
|---|---|---|---|---|---|
| | Bispecific | | Chain 2 | Chain 3 | Chain 4 |
| ID | Antigen 1 | Antigen 2 | (HC1) | (LC1) | (HC2) |
| CLY-023 | CD19 | TCRβV6-5 | 1412 | 1416 | 1417 |
| CLY-024 | CD19 | TCRβV6-5 | 1412 | 1369 | 1418 |
| CLY-018 | CD19 | TCRβV6-5 | 1412 | 1419 | 1417 |
| CLY-019 | CD19 | TCRβV6-5 | 1412 | 1420 | 1417 |
| CLY-020 | CD19 | TCRβV6-5 | 1412 | 1369 | 1421 |
| CLY-021 | CD19 | TCRβV6-5 | 1412 | 1369 | 1422 |
| CLY-022 | CD19 | TRβV6-5 | 1412 | 1369 | 1423 |

### A. Human CD19 and TCRβV6-5-Binding Via ELISA

Bispecific constructs CLY-018 to CLY-024 (CD19XTRBV) were tested for binding against human CD19 (FIGS. 22A-22B) and cyno CD19 (FIGS. 22C-22D). The same bispecific constructs were also tested for binding against human TRBV6-5 (FIGS. 22E-22F) and cyno TRBV6-2 (FIGS. 22G-22H).

### B. Cell Binding by Flow Cytometry

Polypeptide molecules were evaluated in flow cytometry based cell binding assays using human or cynomolgus monkey PBMCs that comprise unmanipulated physiologic ratios of T cells and B cells. Titrated polypeptide molecules were incubated with freshly thawed 250,000 human or cynomolgus monkey PBMCs for 1 hour in serum containing culture medium. Cells were washed and stained with live/dead dye, and CD3, CD8, CD4, TRBV, CD20, and/or CD19 fluorescent antibodies as well as a secondary fluorescent antibody that recognizes polypeptide molecules. Cells were washed again and fixed. Stained and fixed cells were stored at 4° C. until analysis. Mean fluorescent intensity (MFI) of the cell bound polypeptide molecule on T cells or B cells was normalized to the maximum observed and plotted against the logarithmic polypeptide molecule concentration. Where indicated the concentration of polypeptide molecule required to reach half maximum readout was calculated in Graphpad Prism and reported as an EC50. Readouts included EC50 binding on T cells and EC50 binding on B cells from human and cynomolgus monkey.

Results of bispecific polypeptide molecules, CLY-018-CLY-024, evaluated for cell binding by flow cytometry to human B cells are displayed at FIGS. 23A-23B, and to human T cells are displayed at FIGS. 23C-23D. CLY-018-CLY-024 were also evaluated for binding to cyno B cells and cyno T cells as shown in FIGS. 24A-24B and FIGS. 24C-24D, respectively.

### Example 5: Activity of Anti-CD19/Anti-TCRβV6-5 Polypeptide Molecules in Healthy Human and Monkey PBMCs

The objective of this study was to evaluate the ability of anti-CD19/anti-TCRβV6-5 polypeptide molecules to deplete B cells, expand and differentiate T cells, and induce cytokines in human and monkey PBMCs.

### Methods

Polypeptide molecules were evaluated in flow cytometry based functional ex vivo assays using human or cynomolgus monkey PBMCs that comprise unmanipulated physiologic ratios of T cells and B cells. Titrated polypeptide molecules were incubated with freshly thawed 250,000 human or cynomolgus monkey PBMCs per well for up to 7 days. Readouts were taken on days 1, 3, 5, and/or 7 as well as pre-treatment (day 0). At the indicated timepoints supernatants were harvested for cytokine measurements and cells were harvested for flow cytometry measurements. Supernatants from human PBMCs were analyzed for cytokines using a Meso Scale Device kit following the manufacturer's instructions. Supernatants from cynomolgus monkey PBMCs were analyzed using a cytometric bead array following the manufacturer's instructions. Flow cytometry measurements included live/dead, CD3, CD8, CD4, Foxp3, TRBV, CD25, GranzymeB, Ki-67, CCR7, CD45RA, CD95, CD20, and/or CD19 in the panel. Counting beads were included to accurately quantify absolute number of cells present in the samples. Flow cytometric readouts were plotted against the logarithmic concentration of polypeptide molecule. Where indicated the concentration of polypeptide molecule required to reach half maximum readout was calculated in Graphpad Prism and reported as an EC50. Readouts included T cell expansion (change in absolute number of viable T cells or T cell subsets), T cell activation, as well as killing of B cells (change in viable B cell counts). Fold change in T cell counts was calculated as polypeptide molecule target positive T cells (CD3+ or TRBV+) post polypeptide molecule treatment divided by the analogous T cell counts without treatment.

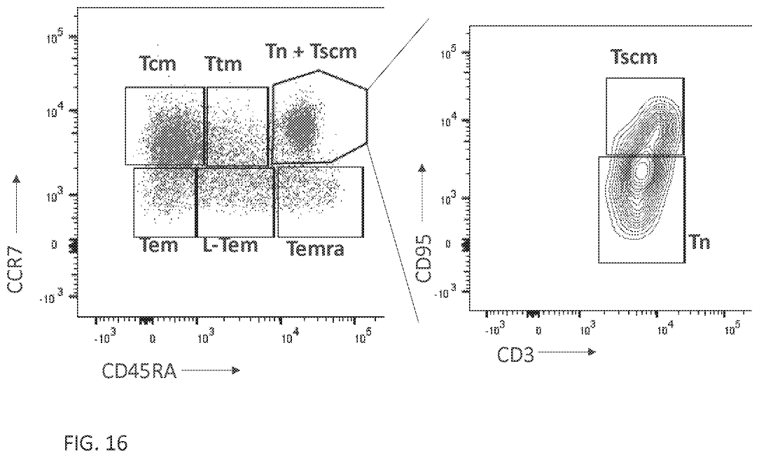

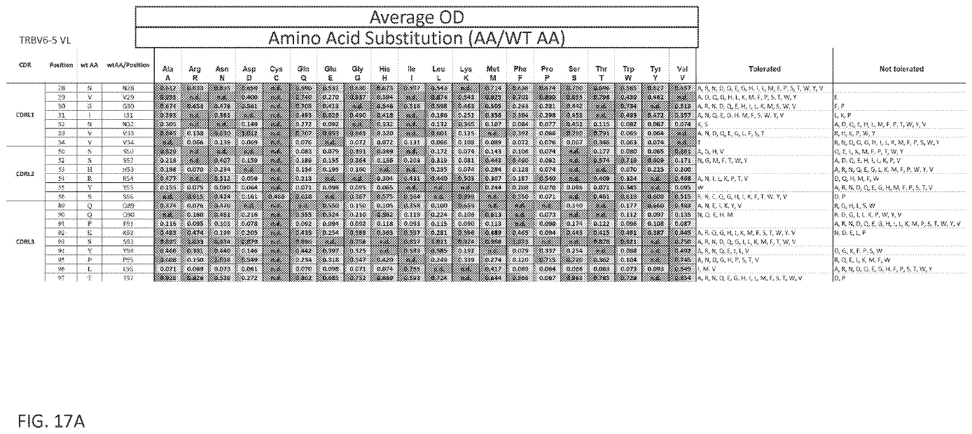

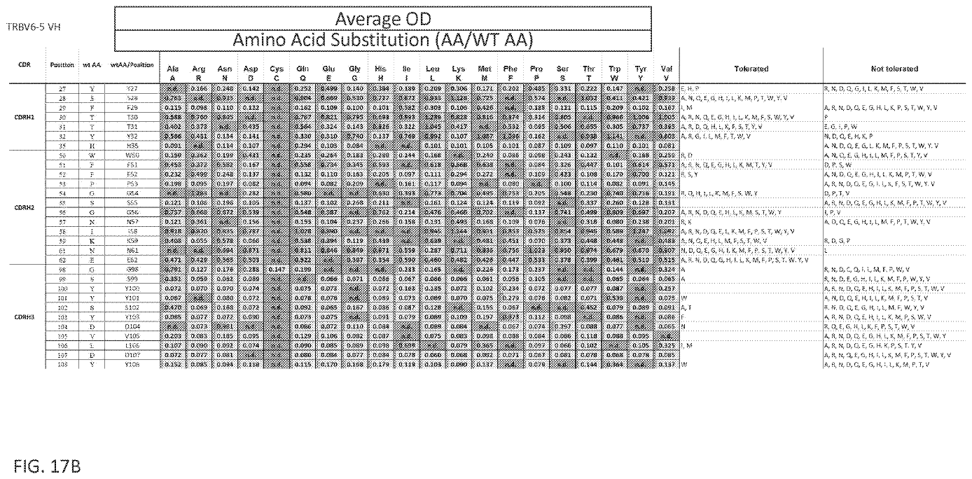

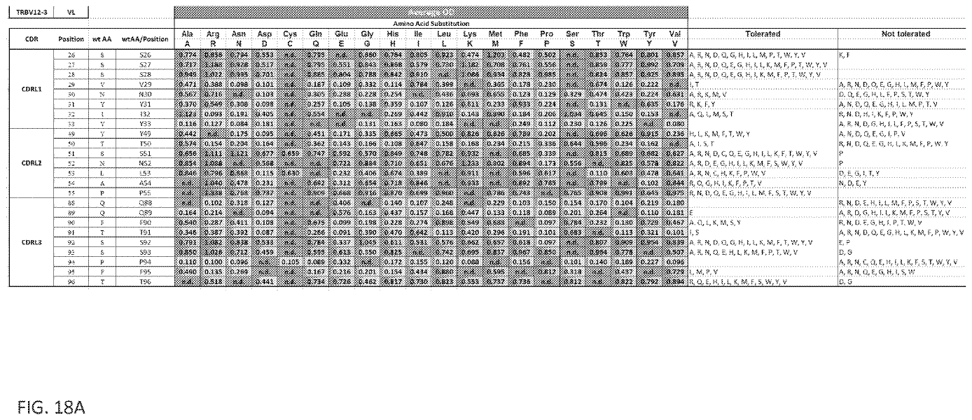

Flow cytometry parameters were established: All viable cells were gated with live/dead stain; and identified as the following: B cells (CD3−CD20+); CD8+ T cells (CD3+CD8+); CD4+ T cells (CD3+CD4+ FoxP3−); Tregs (CD3+CD4+ FoxP3+); Tn cells (CD3+CD8+(or CD4+) CCR7+CD45RA+CD95−); Tscm cells (CD3+CD8+(or CD4+) CCR7+CD45RA+CD95+); Ttm cells (CD3+CD8+(or CD4+) CCR7+CD45RA+/−); Tcm cells (CD3+CD8+(or CD4+) CCR7+CD45RA−); Tem cells (CD3+CD8+(or CD4+) CCR7−CD45RA−); Teff cells (CD3+CD8+(or CD4+) CCR7−CD45RA+/−); and Temra cells (CD3+CD8+(or CD4+) CCR7−CD45RA+). FIG. 16 shows an exemplary flow cytometry plot of the gating strategy used to determine human T cell memory populations.

Results for B-Cell Depletion and TRBV+ Expansion of Human PBMCs with Anti-CD19 Anti-TCRβV6-5 Polypeptide Molecules

Anti-CD19/anti-TCRβV6-5 polypeptide molecules, CLY-018-CLY-023 were evaluated for B cell killing and expansion using human PBMCs. B cell killing results are displayed at FIG. 25A. EC50 values for the human B cell killing assay are displayed at FIG. 25E. CLY-018-CLY-023 were also evaluated for expansion. Results are displayed for CD8+TRBV+(FIG. 25B), non-Treg CD4+ TRBC+(FIG. 25C), and Tregs (FIG. 25D). Displayed results are averages of N=2.

Results for Expansion of TRBV+CD8+ T cell Subsets of Human PBMCs with Anti-CD19 Anti-TCRβV6-5 Polypeptide Molecules

Expansion data for CD8+TRBV+ cells treated with CLY-023 are shown at FIG. 25F. Expansion data for CD8+TRBV+ cells treated with CLY-018 are shown at FIG. 25G. Expansion data for CD8+TRBV+ cells treated with CLY-019 are shown at FIG. 25H. Expansion data for CD8+TRBV+ cells treated with CLY-020 are shown at FIG. 25I. Expansion data for CD8+TRBV+ cells treated with CLY-021 are shown at FIG. 25J. Expansion data for CD8+TRBV+ cells treated with CLY-022 are shown at FIG. 25K. Displayed results are averages of N=2.

Results for B Cell Depletion and TRBV+ Expansion of Cyno (NHP) PBMCs with Anti-CD19 Anti-TCRβV6-5 Polypeptide Molecules

Anti-CD19/anti-TCRβV6-5 polypeptide molecules, CLY-020-CLY-021 and CLY-024 were evaluated for B cell killing and expansion using cyno PBMCs from donors “326” and “1611”. B cell killing results are displayed at FIGS. 25L-25M for donor “326” and at FIG. 25N and FIG. 25O for donor “1611”.

Expansion data for anti-CD19/anti-TCRβV6-5 polypeptide molecules, CLY-020, CLY-021, and CLY-024, is displayed at FIG. 25P for CD8+TRBV+ cyno “326” cells at FIG. 25Q for cyno “1611”. Expansion data for anti-CD19/anti-TCRβV6-5 polypeptide molecules, CLY-020, CLY-021, and CLY-024, is displayed at FIG. 25R for CD4+TRBV+ cyno “326” cells at FIG. 25S for cyno “1611”.

Results of Phenotypic Analysis of Cyno Cells with Anti-CD19 Anti-TCRβV6-5 Polypeptide Molecules

The memory phenotype of cyno cells transformed with the anti-CD19/anti-TCRβV6-5 polypeptide molecule CLY-020 is displayed at FIG. 25T for donor “326” and at FIG. 25U for donor “1611”. The populations: Tn (CD3+CD8+(or CD4+) CCR7+CD45RA+CD95−); Tscm—CD3+CD8+(or CD4+) CCR7+CD45RA+CD95+; Ttm—CD3+CD8+(or CD4+) CCR7+CD45RA+/−; Tcm—CD3+CD8+(or CD4+) CCR7+CD45RA−; Tem—CD3+CD8+(or CD4+) CCR7−CD45RA−; Teff—CD3+CD8+(or CD4+) CCR7−CD45RA+/−; and Temra—CD3+CD8+(or CD4+) CCR7−CD45RA+ were identified for the two cyno donors.

Results of TRBV Specificity with Anti-CD19 Anti-TCRβV6-5 Polypeptide Molecules

Polypeptide molecules were evaluated in TRBV specificity assays based on RNA sequencing of expanded human CD4 or CD8 T cells from three different donors. Plate bound polypeptide molecules were incubated with freshly thawed CD4 or CD8 T cells for 7 days. A no stimulation control was used where T cells were not treated with polypeptide molecules. After 7 days all cells were harvested, counted by flow cytometry, and processed to RNA using a kit following the manufacturer's instructions. RNA was then sequenced using Nanostring with kits focused on the TCR complex including all TRBV and TRAV sequence families. The number of transcripts measured for each individual TRBV or TRAV gene was counted and plotted relative to the no stimulation control. An increase in specific TRBV transcripts relative to the no stimulation control was an indicator of which T cells expanded in the wells during the 7 day polypeptide treatment. The TRBV transcript signal was furthermore an indicator of the specificity of the polypeptide molecule to activate and expand specific TRBV expressing T cells.

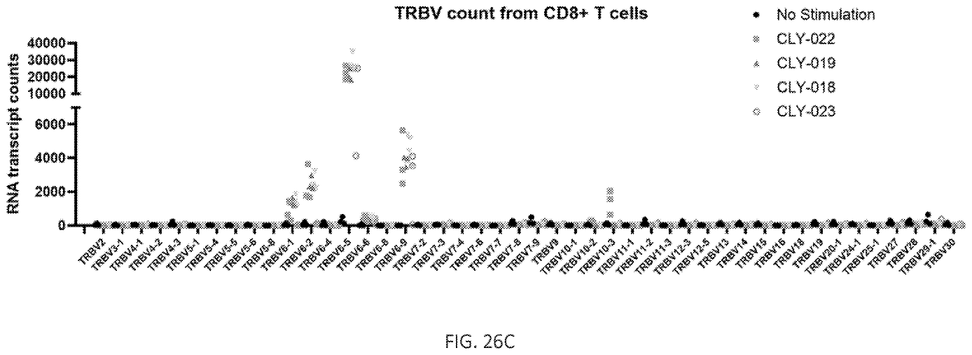

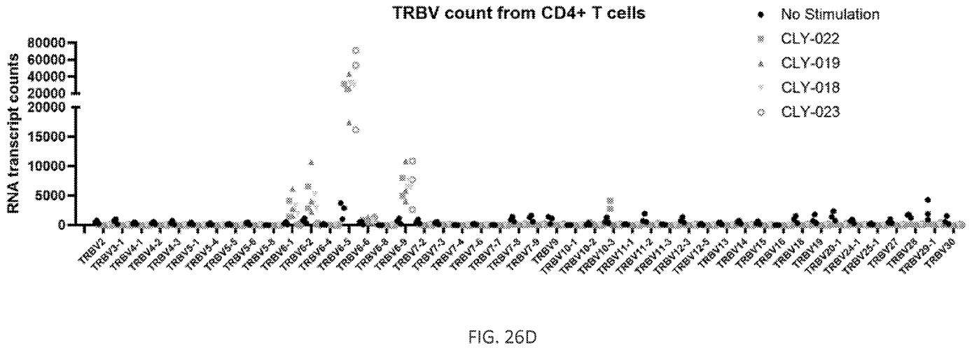

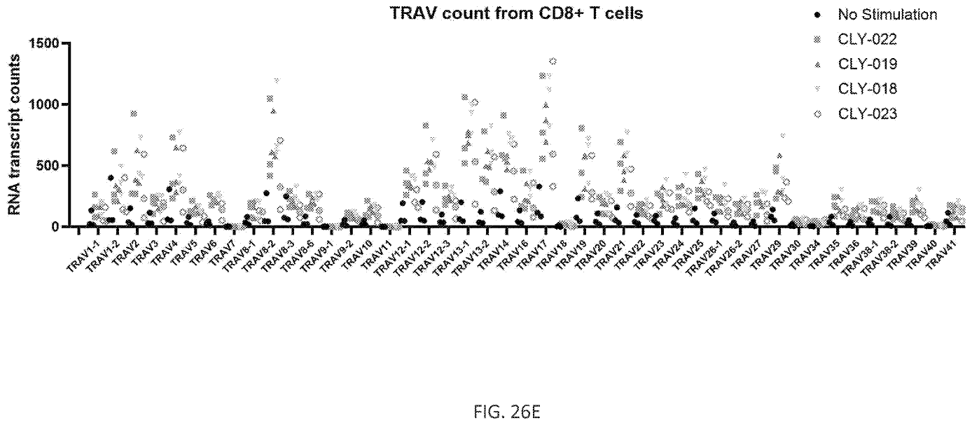

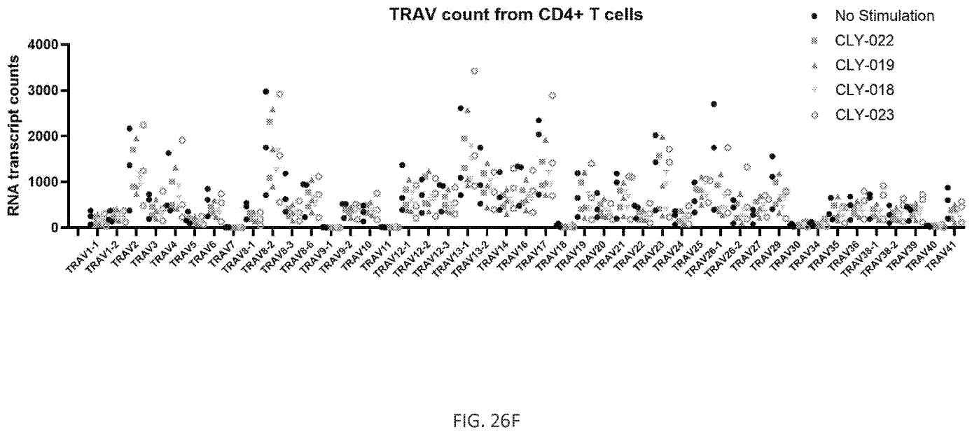

Results of CD4+ or CD8+ T cells using plate-bound constructs CLY-023, CLY-018, CLY-019, or CLY-022 are shown in FIG. 26A (TRBV+CD8+ T cells) or FIG. 26B (TRBV+CD4+ T cells). Results show that plate-bound binding constructs induced CD8 and CD4 T cell expansion in a 7-day culture.

The expansion for TRBV+CD8+ T cells for constructs CLY-023, CLY-018, CLY-019, or CLY-022, after 7-day culture with plate-bound binding constructs is shown at FIG. 26C. The expansion was specific to expansion of TRBV6-1, 6-2, 6-5, and 6-9 (n=3 donor CD8+ T cells). Results demonstrate that CD8 T cells that expanded during the 7 day culture with plate bound polypeptide molecules were enriched for TRBV6-1, 6-2, 6-5, and 6-9.

The expansion for the TRBV+CD4 T cells after 7 day culture with plate bound constructs CLY-023, CLY-018, CLY-019, or CLY-022 are shown in FIG. 26D. Results demonstrate that CD4 T cells that expanded during the 7 day culture with plate bound polypeptide molecules were enriched for TRBV6-1, 6-2, 6-5, and 6-9.

Expansion of TRAV+CD8 T cells after 7 day culture with plate bound binding molecules, CLY-023, CLY-018, CLY-019, or CLY-022 where no change in relative abundance of TRAVs with TRBV stimulation (n=3 donor CD8+ T cells) is shown in FIG. 26E. Results show that the CD8+ T cells that expanded during the 7 day culture with plate bound polypeptide molecules exhibited no bias or enrichment for TRAV. Data is additionally consistent with a lack of bias in TRAV pairing with TRBV6 or TRBV10.

A similar experiment was completed to demonstrate expansion of TRAV+CD4 T cells after 7 day culture with plate bound drug where no change in relative abundance of TRAVs with TRBV stimulation (n=2-3 donor CD4+ T cells) is shown in FIG. 26F. Results show that the CD4+ T cells that expanded during the 7 day culture with plate bound polypeptide molecules exhibited no bias or enrichment for TRAV. Data consistent with a lack of bias in TRAV pairing with TRBV6 or TRBV10.

### Example 6: In Vivo B Cell Depletion and Cytokine Induction with Anti-CD19/Anti-TCRβV6-5 Polypeptide Molecules

The objective of this study was to evaluate the ability of anti-CD19/anti-TCRβV6-5 polypeptide molecules to deplete B cells and induce cytokines in vivo.

Pharmacokinetics and exploratory safety of polypeptide molecules were evaluated in cynomolgus monkeys. Briefly, cynomolgus monkeys of approximately 3 kg bodyweight were administered polypeptides intravenously or subcutaneously and observed daily for signs of adverse events. No in-life adverse events were observed at any timepoint for any polypeptide molecule administered at any dose by any route. After dosing, blood was collected in K₂ EDTA tubes at specific timepoints and processed to plasma for pharmacokinetic analysis. Blood samples were also stained and processed within a couple hours after collection and used for counting B cells by flow cytometry using standard techniques. Plasma was stored frozen until pharmacokinetic and cytokine analysis. Concentration of polypeptide molecules in plasma was measured via standard ELISA techniques relative to a reference standard diluted in control cyno plasma. Plasma concentration curves were fit to a standard two compartment model. Fitting of pharmacokinetics enabled the calculation of Cmax, half-life, volume of distribution, clearance, and area under the curve (AUC). Plasma was also analyzed for cytokines using a non-human primate cytometric Th1/Th2 bead array kit from BD biosciences following the manufacturer's instructions. IFN-γ, TNF-α, IL-6, IL-5, IL-4, and IL-2 levels in plasma were calculated relative to reference standards provided with the bead array kit.

### Results for CLY-023 in Cynomolgus Monkeys

Pharmacokinetics and B cell count data for monkeys dosed with CLY-023 is displayed at FIGS. 27A-27B. Minimal B cell depletion was observed in cynomolgus monkey dosed up to 1 mg/kg IV (FIG. 27B). Plasma concentration of CLY-023 as a function of time is displayed at FIG. 27A.

Cytokine induction data evaluating presence of IFNγ, TNF, IL-2, and IL-6 for the same monkeys is displayed at FIGS. 27C-27D. No in-life observations or signs of cytokine release syndrome were observed in cynomolgus monkeys dosed up to 1 mg/kg IV.

### Results for CLY-024 in Cynomolgus Monkeys

Pharmacokinetics and B cell count data for monkeys dosed with CLY-024 is displayed at FIGS. 27E-27F. As shown in FIG. 27F, moderate B cell depletion observed in cynomolgus monkey dosed at 1 mg/kg IV; minimal B cell depletion observed at lower doses (<1 mg/kg IV). Plasma concentration of CLY-024 as a function of time is displayed at FIG. 27E.

Cytokine induction data for the same monkeys is displayed at FIGS. 27G-27I. No in-life observations or signs of cytokine release syndrome were observed in cynomolgus monkeys dosed up to 1 mg/kg IV.

### Results for CLY-020 in Cynomolgus Monkeys

Pharmacokinetics, B cell count, and cytokine induction data for monkeys dosed with CLY-020 is displayed at FIGS. 27J-27L. The clearance of CLY-020 was faster than the aforementioned binders (FIG. 27J). Deep B cell depletion was observed in cynomolgus monkeys dosed at 1 mg/kg IV (FIG. 27K). No in-life observations or signs of cytokine release syndrome in cynomolgus monkeys dosed at 1 mg/kg IV were detected (FIG. 27L).

### Results for CLY-018 in Cynomolgus Monkeys

Pharmacokinetics and B-cell count data for monkeys dosed with CLY-018 is displayed at FIGS. 27M-27N. Deep B cell depletion was observed in cynomolgus monkeys dosed at 0.1 mg/kg IV or above (FIG. 27N).

Cytokine induction data for the same monkeys is displayed at FIGS. 27O-27R. No in life observations or signs of cytokine release syndrome were observed in cynomolgus monkeys dosed up to 9 mg/kg IV.

### Example 7: B Cell Depletion, T Cell Expansion, and T Cell Phenotyping Using Healthy Human and Autoimmune Disease PBMCs

The objective of this study was to evaluate the ability of anti-CD19/anti-TCRβV6-5 polypeptide molecules to deplete B cells, expand and differentiate T cells, and induce cytokines in healthy human donor PBMCS, as well as PBMCs from rheumatoid arthritis (RA), Systemic lupus erythematosus (SLE), and Myasthenia gravis (MG) autoimmune patients.

Polypeptide molecules were evaluated in flow cytometry based functional ex vivo assays using human PBMCs from autoimmune patients (RA, SLE, MG) that comprise unmanipulated physiologic ratios of T cells and B cells. Titrated polypeptide molecules were incubated with freshly thawed 250,000 human PBMCs per well for up to 7 days. Readouts were taken on days 1, 3, 4, 5, and/or 7 as well as pre-treatment (day 0). At the indicated timepoints supernatants were harvested for cytokine measurements and cells were harvested for flow cytometry measurements. Supernatants from human PBMCs were analyzed for cytokines using a Meso Scale Device kit following the manufacturer's instructions. Supernatants from cynomolgus monkey PBMCs were analyzed using a cytometric bead array following the manufacturer's instructions. Flow cytometry measurements included live/dead, CD3, CD8, CD4, Foxp3, TRBV, CD25, GranzymeB, Ki-67, CCR7, CD45RA, CD95, CD20, and/or CD19 in the panel. Counting beads were included to accurately quantify absolute number of cells present in the samples. Flow cytometric readouts were plotted against the logarithmic concentration of polypeptide molecule. Where indicated the concentration of polypeptide molecule required to reach half maximum readout was calculated in Graphpad Prism and reported as an EC50. Readouts included T cell expansion (change in absolute number of viable T cells or T cell subsets), T cell activation, as well as killing of B cells (change in viable B cell counts). Fold change in T cell counts was calculated as polypeptide molecule target positive T cells (CD3+ or TRBV+) post polypeptide molecule treatment divided by the analogous T cell counts without treatment.

Assays implemented the below gating parameters (All viable cells were gated with live/dead stain): (a) B cells—CD3−CD20+; (b) CD8+ T cells—CD3+CD8+; (c) CD4+ T cells—CD3+CD4+ FoxP3−; (d) Tregs—CD3+CD4+ FoxP3+; (e) Tn—CD3+CD8+(or CD4+) CCR7+CD45RA+CD95−; (f) Tscm—CD3+CD8+(or CD4+) CCR7+CD45RA+CD95+; (g) Ttm—CD3+CD8+(or CD4+) CCR7+CD45RA+/−; (h) Tcm—CD3+CD8+(or CD4+) CCR7+CD45RA−; (i) Tem—CD3+CD8+(or CD4+) CCR7−CD45RA−; (j) Teff—CD3+CD8+(or CD4+) CCR7−CD45RA+/−; and (k) Temra—CD3+CD8+(or CD4+) CCR7−CD45RA+.

Results from Healthy PBMCs

B-cell killing and T-cell expansion results for human healthy donor PBMCs incubated with CLY-018 are displayed at FIG. 28A (B cell fold change), FIG. 28B (CD4+ T cell fold change), FIG. 28C (CD8+ T cell fold change), and FIG. 28D (Treg fold change). Results displayed are for an average of 3 healthy donors (N=3). Expansion according to Tn, Tscm, Ttm, Tcm, Tem, Teff, and Temra CD8+ T cell subsets is shown at FIG. 28E.

Results from Rheumatoid Arthritis (RA) PBMCs

PBMC from 3 rheumatoid arthritis subjects was collected and incubated with CLY-018. Results of B cell killing is shown at FIG. 28F. Results for T cell expansion for CD8+, Treg, and non-Treg CD4 populations is provided in FIG. 28H, FIG. 28I, and FIG. 28G, respectively. Expansion according to Tn, Tscm, Ttm, Tcm, Tem, Teff, and Temra CD8 T cell subsets is shown at FIG. 28J.

Results from Systemic Lupus Erythematosus (SLE) PBMCs

PBMC from 3 SLE subjects was collected and incubated with CLY-018. Results of B cell killing is provided at FIG. 28K. Results of T cell expansion for CD8, Treg, and non-Treg CD4 populations is provided in FIG. 28M, FIG. 28N, and FIG. 28L, respectively. Expansion according to Tn, Tscm, Ttm, Tcm, Tem, Teff, and Temra CD8 T cell subsets is shown at FIG. 28O.

Results from Myasthenia Gravis (MG) PBMCs

PBMC from 3 MG subjects was collected and incubated with CLY-018. Results of B cell killing is provided at FIG. 28P. Results of T cell expansion for CD8, Treg, and non-Treg CD4 populations is provided in FIG. 28R, FIG. 28S, and FIG. 28Q, respectively. Expansion according to Tn, Tscm, Ttm, Tcm, Tem, Teff, and Temra CD8 T cell subsets is shown at FIG. 28T.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

1. A polypeptide molecule comprising an anti-T cell receptor beta variable region (TCRβV)-binding domain, wherein the binding domain comprises:
an IMGT heavy chain complementary determining region 1 (HCDR1) that comprises an amino acid sequence selected from the group consisting of: GYSFTTYY (SEQ ID NO: 114), GYSFTLYY (SEQ ID NO: 139), GYSFTTWY (SEQ ID NO: 146), and GYSFTLWY (SEQ ID NO: 148);
an IMGT heavy chain complementary determining region 2 (HCDR2) that comprises an amino acid sequence selected from the group consisting of: FFPGSGNI (SEQ ID NO: 115) and FFPRSGNI (SEQ ID NO: 143);
an IMGT heavy chain complementary determining region 3 (HCDR3) that comprises an amino acid sequence of: AASYYSFDVLDY (SEQ ID NO: 140);
an IMGT light chain complementary determining region 1 (LCDR1) that comprises an amino acid sequence selected from the group consisting of: QNVGIN (SEQ ID NO: 113) and QNVGIR (SEQ ID NO: 138);
an IMGT light chain complementary determining region 2 (LCDR2) that comprises an amino acid sequence of: SSS; and
an IMGT light chain complementary determining region 3 (LCDR3) that comprises an amino acid sequence selected from the group consisting of: QQFKSYPIT (SEQ ID NO: 135) and QQFKFYPIT (SEQ ID NO: 141);
wherein each of the HCDR1, HCDR2, HCDR3, LCDR1, LCDR2, and/or LCDR3 independently comprise 0, 1, 2, or 3 amino acid mutations.

2. The polypeptide molecule of claim 1, wherein the anti-TCRβV-binding domain comprises:
(i) the HCDR1, the HCDR2, and the HCDR3 comprising the amino acid sequences of SEQ ID NOs: 114, 143, and 140, respectively, and the LCDR1, the LCDR2, and the LCDR3 comprising the amino acid sequences of SEQ ID NOs: 138, SSS, and 141, respectively;
(ii) the HCDR1, the HCDR2, and the HCDR3 comprising the amino acid sequences of SEQ ID NOs: 114, 115, and 140, respectively, and the LCDR1, the LCDR2, and the LCDR3 comprising the amino acid sequences of SEQ ID NOs: 138, SSS, and 135, respectively;
(iii) the HCDR1, the HCDR2, and the HCDR3 comprising the amino acid sequences of SEQ ID NOs: 114, 143, and 140, respectively, and the LCDR1, the LCDR2, and the LCDR3 comprising the amino acid sequences of SEQ ID NOs: 138, SSS, and 135, respectively;
(iv) the HCDR1, the HCDR2, and the HCDR3 comprising the amino acid sequences of SEQ ID NOs: 148, 115, and 140, respectively, and the LCDR1, the LCDR2, and the LCDR3 comprising the amino acid sequences of SEQ ID NOs: 138, SSS, and 135, respectively;
(v) the HCDR1, the HCDR2, and the HCDR3 comprising the amino acid sequences of SEQ ID NOs: 148, 143, and 140, respectively, and the LCDR1, the LCDR2, and the LCDR3 comprising the amino acid sequences of SEQ ID NOs: 113, SSS, and 135, respectively;
(vi) the HCDR1, the HCDR2, and the HCDR3 comprising the amino acid sequences of SEQ ID NOs: 139, 143, and 140, respectively, and the LCDR1, the LCDR2, and the LCDR3 comprising the amino acid sequences of SEQ ID NOs: 138, SSS, and 135, respectively;
(vii) the HCDR1, the HCDR2, and the HCDR3 comprising the amino acid sequences of SEQ ID NOs: 114, 143, and 140, respectively, and the LCDR1, the LCDR2, and the LCDR3 comprising the amino acid sequences of SEQ ID NOs: 113, SSS, and 135, respectively;
(viii) the HCDR1, the HCDR2, and the HCDR3 comprising the amino acid sequences of SEQ ID NOs: 114, 143, and 140, respectively, and the LCDR1, the LCDR2, and the LCDR3 comprising the amino acid sequences of SEQ ID NOs: 138, SSS, and 135, respectively;
(ix) the HCDR1, the HCDR2, and the HCDR3 comprising the amino acid sequences of SEQ ID NOs: 139, 143, and 140, respectively, and the LCDR1, the LCDR2, and the LCDR3 comprising the amino acid sequences of SEQ ID NOs: 138, SSS, and 135, respectively;
(x) the HCDR1, the HCDR2, and the HCDR3 comprising the amino acid sequences of SEQ ID NOs: 139, 115, and 140, respectively, and the LCDR1, the LCDR2, and the LCDR3 comprising the amino acid sequences of SEQ ID NOs: 138, SSS, and 135, respectively;
(xi) the HCDR1, the HCDR2, and the HCDR3 comprising the amino acid sequences of SEQ ID NOs: 146, 115, and 140, respectively, and the LCDR1, the LCDR2, and the LCDR3 comprising the amino acid sequences of SEQ ID NOs: 138, SSS, and 141, respectively;
(xii) the HCDR1, the HCDR2, and the HCDR3 comprising the amino acid sequences of SEQ ID NOs: 148, 115, and 140, respectively, and the LCDR1, the LCDR2, and the LCDR3 comprising the amino acid sequences of SEQ ID NOs: 138, SSS, and 141, respectively;
(xiii) the HCDR1, the HCDR2, and the HCDR3 comprising the amino acid sequences of SEQ ID NOs: 148, 115, and 140, respectively, and the LCDR1, the LCDR2, and the LCDR3 comprising the amino acid sequences of SEQ ID NOs: 138, SSS, and 135, respectively;
(xiv) the HCDR1, the HCDR2, and the HCDR3 comprising the amino acid sequences of SEQ ID NOs: 114, 143, and 140, respectively, and the LCDR1, the LCDR2, and the LCDR3 comprising the amino acid sequences of SEQ ID NOs: 138, SSS, and 141, respectively;
(xv) the HCDR1, the HCDR2, and the HCDR3 comprising the amino acid sequences of SEQ ID NOs: 139, 115, and 140, respectively, and the LCDR1, the LCDR2, and the LCDR3 comprising the amino acid sequences of SEQ ID NOs: 138, SSS, and 141, respectively;
(xvi) the HCDR1, the HCDR2, and the HCDR3 comprising the amino acid sequences of SEQ ID NOs: 148, 143, and 140, respectively, and the LCDR1, the LCDR2, and the LCDR3 comprising the amino acid sequences of SEQ ID NOs: 138, SSS, and 135, respectively;
(xvii) the HCDR1, the HCDR2, and the HCDR3 comprising the amino acid sequences of SEQ ID NOs: 139, 143, and 140, respectively, and the LCDR1, the LCDR2, and the LCDR3 comprising the amino acid sequences of SEQ ID NOs: 138, SSS, and 135, respectively; or
(xviii) the HCDR1, the HCDR2, and the HCDR3 comprising the amino acid sequences of SEQ ID NOs: 148, 143, and 140, respectively, and the LCDR1, the LCDR2, and the LCDR3 comprising the amino acid sequences of SEQ ID NOs: 138, SSS, and 141, respectively.

3. The polypeptide molecule of claim 1, wherein the anti-TCRβV-binding domain comprises an immunoglobulin heavy chain variable (VH) region and/or an immunoglobulin light chain variable (VL) region.

4. The polypeptide molecule of claim 3, wherein the VH region comprises an amino acid sequence that is at least about 80%, at least about 85%, at least about 90%, at least about 95%, at least about 96%, at least about 97%, at least about 98%, at least about 99%, or 100% identical to an amino acid sequence selected from the group consisting of: SEQ ID NOs: 100-111, 2512, and 2513.

5. The polypeptide molecule of claim 3, wherein the VL region comprises an amino acid sequence that is at least about 80%, at least about 85%, at least about 90%, at least about 95%, at least about 96%, at least about 97%, at least about 98%, at least about 99%, or 100% identical to an amino acid sequence selected from the group consisting of: SEQ ID NOs: 85-93, and 2514.

6. The polypeptide molecule of claim 3, wherein the VH region comprises an amino acid sequence that is at least about 80%, at least about 85%, at least about 90%, at least about 95%, at least about 96%, at least about 97%, at least about 98%, at least about 99%, or 100% identical to an amino acid sequence selected from the group consisting of: SEQ ID NOs: 100-111, 2512, and 2513; and the VL region comprises an amino acid sequence that is at least about 80%, at least about 85%, at least about 90%, at least about 95%, at least about 96%, at least about 97%, at least about 98%, at least about 99%, or 100% identical to an amino acid sequence selected from the group consisting of: SEQ ID NOs: 85-93 and 2514.

7. The polypeptide molecule of claim 1, wherein the anti-TCRβV-binding domain is a single chain variable fragment (scFv), Fab, CrossFab, Fab′, or F(ab′)₂.

8. The polypeptide molecule of claim 1, wherein the anti-TCRβV-binding domain is an scFv comprising a VH region and a VL region.

9. The polypeptide molecule of claim 8, wherein the C-terminus of the VH region is linked to the N-terminus of the VL region.

10. The polypeptide molecule of claim 8, wherein the C-terminus of the VL region is linked to the N-terminus of the VH region.

11. The polypeptide molecule of claim 8, wherein the VH region and VL region are linked by a peptide linker.

12. The polypeptide molecule of claim 11, wherein the peptide linker comprises the formula of (Gly4Ser)ₙ, wherein n=1-5 (SEQ ID NO: 1424).

13. The polypeptide molecule of claim 11, wherein the peptide linker is (Gly₄Ser)₃ (SEQ ID NO: 1342).

14. The polypeptide molecule of claim 8, wherein the VH region and the VL region are linked by one or more engineered disulfide bonds.

15. The polypeptide molecule of claim 14, wherein one cysteine that forms the engineered disulfide bond is located in the VH region, and wherein the other cysteine that forms the engineered disulfide bond is located in the VL region.

16. The polypeptide molecule of claim 14, wherein the positions of the one or more engineered disulfide bonds are selected from the group consisting of: VH44-VL100, VH100B-VL49, VH100-VL50, VH101-VL46, and VH105-VL43, according to EU numbering.

17. The polypeptide molecule of claim 7, wherein the anti-TCRβV-binding domain is an scFv, and the scFv comprises an amino acid sequence that is at least about 80%, at least about 85%, at least about 90%, at least about 95%, at least about 96%, at least about 97%, at least about 98%, at least about 99%, or 100% identical to an amino acid sequence selected from the group consisting of: SEQ ID NOs: 149-222.

18. The polypeptide molecule of claim 7, wherein the anti-TCRβV-binding domain is a Fab comprising the VH region, the VL region, a first heavy chain constant region (CH1), and a light chain constant region (CL), wherein the C-terminus of the VH region is connected to the N-terminus of the CH1 region, and the C-terminus of the VL region is connected to the N-terminus of the CL region.

19. The polypeptide molecule of claim 18, wherein the CH1 region and the CL region are connected by a disulfide bond.

20. The polypeptide molecule of claim 18, wherein TCRβV-binding domain is a charge steering Fab.

21. The polypeptide molecule of claim 20, wherein the CH1 region of the charge steering Fab comprises mutations K147E and/or K213E according to EU numbering, and the CL region of the charge steering Fab comprises mutations E123R and/or Q124K according to EU numbering.

22. The polypeptide molecule of claim 7, wherein the anti-TCRβV-binding domain is a CrossFab comprising the VH region, the VL region, a first heavy chain constant region (CH1), and a light chain constant region (CL), wherein the C-terminus of the VH region is connected to the N-terminus of the CL region, and the C-terminus of the VL region is connected to the N-terminus of the CH1 region.

23. The polypeptide molecule of claim 22, wherein the CH1 region and the CL region are connected by a disulfide bond.

24. The polypeptide molecule of claim 22, wherein TCRβV-binding domain is a charge steering CrossFab.

25. The polypeptide molecule of claim 24, wherein the CH1 region of the charge steering CrossFab comprises mutations K147E and/or K213E according to EU numbering, and the CL region of the charge steering Fab or charge steering CrossFab comprises mutations E123R and/or Q124K according to EU numbering.

26. The polypeptide molecule of claim 7, wherein the anti-TCRβV-binding domain comprises a Fab or a CrossFab, and the Fab or CrossFab comprises (i) a light chain sequence that is at least about 80%, at least about 85%, at least about 90%, at least about 95%, at least about 96%, at least about 97%, at least about 98%, at least about 99%, or 100% identical to an amino acid sequence selected from the group consisting of: SEQ ID NOs: 230-574; and (ii) a heavy chain sequence that is at least about 80%, at least about 85%, at least about 90%, at least about 95%, at least about 96%, at least about 97%, at least about 98%, at least about 99%, or 100% identical to an amino acid sequence selected from the group consisting of: SEQ ID NOs: 575-1084.

27. A recombinant nucleic acid molecule encoding the polypeptide molecule of claim 1.

28. A vector comprising the recombinant nucleic acid molecule of claim 27.

29. A composition comprising the polypeptide molecule of claim 1.

30. A pharmaceutical composition comprising (a) the polypeptide molecule of claim 1, and (b) a pharmaceutically acceptable carrier, diluent, or excipient.

31. A method of producing a polypeptide, comprising introducing the recombinant nucleic acid molecule of claim 27, into a host cell under conditions that express the polypeptide molecule in the host cell.

32. A method of expanding a T cell population, wherein the method comprises contacting the T cell population with the polypeptide molecule of claim 1, wherein the T cell population expresses a TCRβV antigen on the cell surface, and wherein the polypeptide molecule specifically binds to the TCRβV antigen.

33. A method of treating a disease, disorder, or condition in a subject in need thereof, comprising administering to the subject an effective amount of the polypeptide molecule of claim 1.

34. A polypeptide molecule comprising an anti-TCRβV-binding domain, wherein the binding domain comprises:
(a) an IMGT HCDR1 that comprises the amino acid sequence of G₂₆Y₂₇X₂₈F₂₉X₃₀X₃₁X₃₂Y₃₃, wherein X₂₈ is selected from the group consisting of S, K, A, P, T, and V; X₃₀ is selected from the group consisting of T, V, L, Y, W, and Q; X₃₁ is selected from the group consisting of: T and L; and X₃₂ is selected from the group consisting of Y, F, M, W, L, R, and T;
an IMGT HCDR2 that comprises the amino acid sequence of F₅₁F₅₂P₅₃X₅₄S₅₅G₅₆N₅₇X₅₈, wherein X₅₄ is selected from the group consisting of G and R; and X₅₈ is selected from the group consisting of: I, V, Y, L, A, Q, K, T, and M;
an IMGT HCDR3 that comprises the amino acid sequence of A₉₇X₉₈S₉₉Y₁₀₀Y₁₀₁S₁₀₂X₁₀₃D₁₀₄V₁₀₅L₁₀₆D₁₀₇Y₁₀₈, wherein X₉₈ is selected from the group consisting of: G and A; and X₁₀₃ is selected from the group consisting of: Y and F;
an IMGT LCDR1 that comprises the amino acid sequence of Q₂₇N₂₈Z₂₉G₃₀I₃₁Z₃₂, wherein Z₂₉ is selected from the group consisting of V and G; and wherein Z₃₂ is selected from the group consisting of N and R;
an IMGT LCDR2 that comprises the amino acid sequence of S₅₀S₅₁S₅₂; and
an IMGT LCDR3 that comprises the amino acid sequence of Q₈₉Q₉₀F₉₁K₉₂Z₉₃Y₉₄Z₉₅Z₉₆Z₉₇, wherein Z₉₃ is selected from the group consisting of S, F, R, A, and L; Z₉₅ is selected from the group consisting of P and N; Z₉₆ is selected from the group consisting of L and I; and Z₉₇ is selected from the group consisting of: T, G, and A;
wherein the polypeptide molecule is not one in which i) the HCDR1, the HCDR2, and the HCDR3 are all selected from the group consisting of: SEQ ID NOs: 114-116 and 2458-2499; and ii) wherein the LCDR1, the LCDR2, and the LCDR3 are all selected from the group consisting of: SEQ ID NOs: 73, 113, and 2500-2508, and SSS; or
(b) a Kabat HCDR1 that comprises the amino acid sequence of X₃₁X₃₂YIH, wherein X₃₁ is selected from the group consisting of: T and L; and X₃₂ is selected from the group consisting of Y, F, M, W, L, R, and T;
a Kabat HCDR2 that comprises the amino acid sequence of W₅₀F₅₁F₅₂P₅₃X₅₄S₅₅G₅₆N₅₇X₅₈K₅₉Y₆₀X₆₁E₆₂K₆₃F₆₄K₆₅G₆₆, wherein X₅₄ is selected from the group consisting of G and R; X₅₈ is selected from the group consisting of: I, V, Y, L, A, Q, K, T, and M; and X₆₁ is selected from the group consisting of N, V, S, H, P, T, and D;
a Kabat HCDR3 that comprises the amino acid sequence of S₉₉Y₁₀₀Y₁₀₁S₁₀₂X₁₀₃D₁₀₄V₁₀₅L₁₀₆D₁₀₇Y₁₀₈, wherein X₁₀₃ is selected from the group consisting of: Y and F;
a Kabat LCDR1 that comprises the amino acid sequence of K₂₄A₂₅S₂₆Q₂₇N₂₈Z₂₉G₃₀I₃₁Z₃₂Z₃₃V₃₄, wherein Z₂₉ is selected from the group consisting of V and G; Z₃₂ is selected from the group consisting of N and R; and Z₃₃ is selected from the group consisting of V and D;
a Kabat LCDR2 that comprises the amino acid sequence of S₅₀S₅₁S₅₂H₅₃R₅₄Y₅₅Z₅₆, wherein Z₅₆ is selected from the group consisting of: S and R; and
a Kabat light chain CDR3 (LCDR3) that comprises the amino acid sequence of Q₈₉Q₉₀F₉₁K₉₂Z₉₃Y₉₄Z₉₅Z₉₆Z₉₇, wherein Z₉₃ is selected from the group consisting of S, F, R, A, and L; Z₉₅ is selected from the group consisting of P and N; Z₉₆ is selected from the group consisting of L and I; and Z₉₇ is selected from the group consisting of T, G, and A;
wherein the polypeptide molecule is not one in which i) the HCDR1, the HCDR2, and the HCDR3 are all selected from the group consisting of: SEQ ID NOs: 69, 70, 112, and 2416-2447; and ii) wherein the LCDR1, the LCDR2, and the LCDR3 are all selected from the group consisting of: SEQ ID NOs: 71-73 and 2448-2457.

35. A polypeptide molecule comprising an anti-TCRβV-binding domain, wherein the binding comprises:
a Kabat HCDR1 that comprises an amino acid sequence selected from the group consisting of: TYYIH (SEQ ID NO: 112), LYYIH (SEQ ID NO: 136), TWYIH (SEQ ID NO: 145), and LWYIH (SEQ ID NO: 147);
a Kabat HCDR2 that comprises an amino acid sequence selected from the group consisting of: WFFPGSGNIKYNEKFKG (SEQ ID NO: 69) and WFFPRSGNIKYNEKFKG (SEQ ID NO: 142);
a Kabat HCDR3 that comprises an amino acid sequence of SYYSFDVLDY (SEQ ID NO: 137);
a Kabat LCDR1 that comprises an amino acid sequence selected from the group consisting of: KASQNVGINVV (SEQ ID NO: 71) and KASQNVGIRVV (SEQ ID NO: 134);
a Kabat LCDR2 that comprises an amino acid sequence selected from the group consisting of: SSSHRYS (SEQ ID NO: 72), SSSHRYK (SEQ ID NO: 128), and SSSHRYR (SEQ ID NO: 144); and
a Kabat LCDR3 that comprises an amino acid sequence selected from the group consisting of: QQFKSYPIT (SEQ ID NO: 135) and QQFKFYPIT (SEQ ID NO: 141);
wherein each of the HCDR1, HCDR2, HCDR3, LCDR1, LCDR2, and/or LCDR3 independently comprise 0, 1, 2, or 3 amino acid mutations.

36. A polypeptide molecule comprising an anti-TCRβV-binding domain, wherein the binding domain comprises:
an IMGT HCDR1 that comprises an amino acid sequence selected from the group consisting of: GFTFSIFG (SEQ ID NO: 1188), GFTFSRTG (SEQ ID NO: 1189), GFTFSIKG (SEQ ID NO: 1190), GFTFSNKG (SEQ ID NO: 1191), GFTFSRFG (SEQ ID NO: 1192), GFTFSRKG (SEQ ID NO: 1193), GFTFSITG (SEQ ID NO: 1194), GFTFSIYG (SEQ ID NO: 1195), and GFTFSNTG (SEQ ID NO: 1196);
an IMGT HCDR2 that comprises an amino acid sequence selected from the group consisting of: ISSGSSTI (SEQ ID NO: 1197), ISSTSSTI (SEQ ID NO: 1198), ISSSSSTI (SEQ ID NO: 1199), and ISSWSSTI (SEQ ID NO: 1200);
an IMGT HCDR3 that comprises an amino acid sequence of ARRGEGAMDY (SEQ ID NO: 1201) or ARRGAGAMDY (SEQ ID NO: 1202);
an IMGT LCDR1 that comprises an amino acid sequence selected from the group consisting of: SSVNY (SEQ ID NO: 1182), RSVNY (SEQ ID NO: 1184), and KSVNY (SEQ ID NO: 1185);
an IMGT LCDR2 that comprises an amino acid sequence of YTS; and
an IMGT LCDR3 that comprises an amino acid sequence selected from the group consisting of: QQFTSSPFT (SEQ ID NO: 1157) and QQFTSSPFV (SEQ ID NO: 1159);
wherein each of the HCDR1, HCDR2, HCDR3, LCDR1, LCDR2, and/or LCDR3 independently comprise 0, 1, 2, or 3 amino acid mutations.

37. A polypeptide molecule comprising an anti-TCRβV-binding domain, wherein the binding domain comprises:
a Kabat HCDR1 that comprises an amino acid sequence selected from the group consisting of: NFGMH (SEQ ID NO: 1160); IFGMH (SEQ ID NO: 1162); RTGMH (SEQ ID NO: 1163); IKGMH (SEQ ID NO: 1164); NKGMH (SEQ ID NO: 1165); RFGMH (SEQ ID NO: 1166); RKGMH (SEQ ID NO: 1167); ITGMH (SEQ ID NO: 1168); IYGMH (SEQ ID NO: 1169); and NTGMH (SEQ ID NO: 1170);
a Kabat HCDR2 that comprises an amino acid sequence selected from the group consisting of: YISSGSSTIYYADTLKG (SEQ ID NO: 1171), YISSTSSTILYADTLKG (SEQ ID NO: 1173), YISSSSSTILYADTLKG (SEQ ID NO: 1174), YISSSSSTIYYADTLKG (SEQ ID NO: 1175), YISSWSSTILYADTLKG (SEQ ID NO: 1176), YISSWSSTIYYADTLKG (SEQ ID NO: 1177), YISSTSSTIYYADTLKG (SEQ ID NO: 1178), and YISSGSSTILYADTLKG (SEQ ID NO: 1179);
a Kabat HCDR3 that comprises an amino acid sequence of RGEGAMDY (SEQ ID NO: 1180) or RGAGAMDY (SEQ ID NO: 1181);
a Kabat LCDR1 that comprises an amino acid sequence selected from the group consisting of: RASSSVNYIY (SEQ ID NO: 1148), RASRSVNYAY (SEQ ID NO: 1151), RASKSVNYAY (SEQ ID NO: 1152), RASKSVNYIY (SEQ ID NO: 1153), RASSSVNYAY (SEQ ID NO: 1154), and RASRSVNYIY (SEQ ID NO: 1155);
a Kabat LCDR2 that comprises an amino acid sequence of YTSNLAP (SEQ ID NO: 1156); and
a Kabat LCDR3 that comprises an amino acid sequence selected from the group consisting of: QQFTSSPFT (SEQ ID NO: 1157) and QQFTSSPFV (SEQ ID NO: 1159);
wherein each of the HCDR1, HCDR2, HCDR3, LCDR1, LCDR2, and/or LCDR3 independently comprise 0, 1, 2, or 3 amino acid mutations.

38. A polypeptide molecule comprising an anti-TCRβV, wherein the binding molecule comprises:
(a) an IMGT HCDR1 that comprises the amino acid sequence of G₂₆F₂₇X₂₈F₂₉X₃₀X₃₁X₃₂G₃₃, wherein X₂₈ is selected from the group consisting of T and K; X₃₀ is selected from the group consisting of S and H; X₃₁ is selected from the group consisting of: N, R, I, Y, V, H, L, T, S, Q, and D; and X₃₂ is selected from the group consisting of F, K, T, Y, V, R, A, H, and I;
an IMGT HCDR2 that comprises the amino acid sequence of I₅₁S₅₂S₅₃X₅₄X₅₅S₅₆X₅₇X₅₈, wherein X₅₄ is selected from the group consisting of G, W, S, Y, T, Q, V, R, H, E, M, K, and P;
X₅₅ is selected from the group consisting of S and H; X₅₇ is selected from the group consisting of T and V; and X₅₈ is selected from the group consisting of: I, F, W, and Y;
an IMGT HCDR3 that comprises the amino acid sequence of A₉₇R₉₈R₉₉G₁₀₀X₁₀₁G₁₀₂A₁₀₃M₁₀₄D₁₀₅X₁₀₆, wherein X₁₀₁ is selected from the group consisting of: E, H, L, A, T, M, R, S, G, N, Y, W, K, I, and P; and X₁₀₆ is selected from the group consisting of: Y, N, A, W, V, M, P, and T;
an IMGT LCDR1 that comprises the amino acid sequence of Z₂₇Z₂₈I₂₉Z₃₀Z₃₁, wherein Z₂₇ is selected from the group consisting of: S, K, R, and H; Z₂₈ is selected from the group consisting of: S, K, N, R, and P; Z₃₀ is selected from the group consisting of: N and V; and Z₃₁ is selected from the group consisting of Y and F;
an IMGT LCDR2 that comprises the amino acid sequence of Z₄₉Z₅₀Z₅₁, wherein Z₄₉ is selected from the group consisting of: Y and K; Z₅₀ is selected from the group consisting of: T and P; and Z₅₁ is selected from the group consisting of S, N, K, R, P, D, and L; and
an IMGT LCDR3 that comprises the amino acid sequence of Q₈₈Q₈₉F₉₀T₉₁Z₉₂Z₉₃P₉₄F₉₅Z₉₆, wherein Z₉₂ is selected from the group consisting of: S, G, and R; Z₉₃ is selected from the group consisting of S, K, A, F, R, P, and H; and Z₉₆ is selected from the group consisting of T, V, S, H, and Y; and
wherein the polypeptide molecule is not one in which i) the HCDR1, the HCDR2, and the HCDR3 are all selected from the group consisting of: SEQ ID NOs: 1186, 1197, and 1201; and ii) the LCDR1, the LCDR2, and the LCDR3 are all selected from the group consisting of: SEQ ID NOs: 1157, 1182, and YTS; or
(b) a Kabat HCDR1 that comprises the amino acid sequence of X₃₁X₃₂G₃₃M₃₄H₃₅, wherein X₃₁ is selected from the group consisting of: N, R, I, Y, V, H, L, T, S, Q, and D; and X₃₂ is selected from the group consisting of F, K, T, Y, V, R, A, H, and I;
a Kabat HCDR2 that comprises the amino acid sequence of Y₅₀I₅₁S₅₂S₅₃X₅₄X₅₅S₅₆X₅₇X₅₈X₅₉Y₆₀X₆₁X₆₂X₆₃L₆₄K₆₅G₆₆, wherein X₅₄ is selected from the group consisting of G, W, S, Y, T, Q, V, R, H, E, M, K, and P; X₅₅ is selected from the group consisting of S and H; X₅₇ is selected from the group consisting of T and V; X₅₈ is selected from the group consisting of: I, F, W, and Y; X₅₉ is selected from the group consisting of: Y, L, I, and H; X₆₁ is selected from the group consisting of A, K, R, D, Q, W, N, S, Y, G, and F; X₆₂ is selected from the group consisting of D, P, and T; and X₆₃ is selected from the group consisting of T, I, K, N, H, E, G, M, R, Y, V, F, L, and P;
a Kabat HCDR3 that comprises the amino acid sequence of R₉₉G₁₀₀X₁₀₁G₁₀₂A₁₀₃M₁₀₄D₁₀₅X₁₀₆, wherein X₁₀₁ is selected from the group consisting of: E, H, L, A, T, M, R, S, G, N, Y, W, K, I, and P; and X₁₀₆ is selected from the group consisting of: Y, N, A, W, V, M, P, and T;
a Kabat LCDR1 that comprises the amino acid sequence of R₂₄A₂₅Z₂₆Z₂₇Z₂₈I₂₉Z₃₀Z₃₁Z₃₂Y₃₃, wherein Z₂₆ is selected from the group consisting of: S, M, V, and L; Z₂₇ is selected from the group consisting of S, K, R, and H; Z₂₈ is selected from the group consisting of: S, K, N, R, and P; Z₃₀ is selected from the group consisting of: N and V; Z₃₁ is selected from the group consisting of Y and F; Z₃₂ is selected from the group consisting of: I, A, and S;
a Kabat LCDR2 that comprises the amino acid sequence of Z₄₉Z₅₀Z₅₁Z₅₂Z₅₃Z₅₄Z₅₅, wherein Z₄₉ is selected from the group consisting of: Y and K; Z₅₀ is selected from the group consisting of: T and P; Z₅₁ is selected from the group consisting of: S, N, K, R, P, D, and L; Z₅₂ is selected from the group consisting of N, R, K, A, and L; Z₅₃ is selected from the group consisting of L and H; Z₅₄ is selected from the group consisting of: A, R, and V; and Z₅₅ is selected from the group consisting of P, R, T, Q, V, and L; and
a Kabat LCDR3 that comprises the amino acid sequence of Q₈₈Q₈₉F₉₀T₉₁Z₉₂Z₉₃P₉₄F₉₅Z₉₆, wherein Z₉₂ is selected from the group consisting of: S, G, and R; Z₉₃ is selected from the group consisting of S, K, A, F, R, P, and H; and Z₉₆ is selected from the group consisting of T, V, S, H, and Y;
wherein the polypeptide molecule is not one in which i) the HCDR1, the HCDR2, and the HCDR3 are all selected from the group consisting of: SEQ ID NOs: 1160, 1171, and 1180; and ii) the LCDR1, the LCDR2, and the LCDR3 are all selected from the group consisting of: SEQ ID NOs: 1148, 1156, and 1157.
